(19) United States
(12) Patent Application Publication
(10) Pub. No.: US 2023/0002372 A1
(43) Pub. Date: Jan. 5, 2023

(54) AZAINDOLE CARBOXAMIDE COMPOUNDS FOR THE TREATMENT OF MYCOBACTERIAL INFECTIONS

(71) Applicant: THE GLOBAL ALLIANCE FOR TB DRUG DEVELOPMENT, INC., New York, NY (US)

(72) Inventors: Takushi KANEKO, Guilford, CT (US); Nader FOTOUHI, Basking Ridge, NJ (US)

(21) Appl. No.: 17/761,821

(22) PCT Filed: Sep. 25, 2020
(86) PCT No.: PCT/US2020/052934

§ 371 Date: Mar. 18, 2022

Related U.S. Application Data
(60) Provisional application No. 62/906,461, filed on Sep. 26, 2019.

(51) Int. Cl. C07D 471/04; C07F 7/08; A61K 45/06

(52) U.S. Cl. CPC C07D 471/04; C07F 7/0816; A61K 45/06

(57) ABSTRACT

Provided herein are compounds of Formula (I) and Formula (II): as well as pharmaceutically acceptable salts thereof, wherein the substituents are as those disclosed in the specification. These compounds, and the pharmaceutical compositions containing them, are useful for the treatment of tuberculosis.

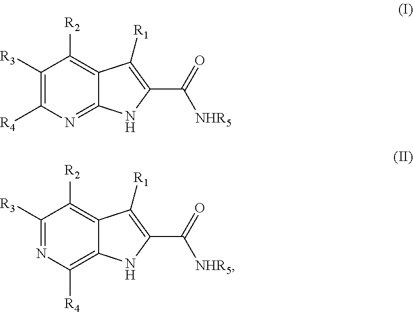

### FIELD OF THE INVENTION

The invention is directed to, for example, compounds of Formula (I) and compounds of Formula (II):

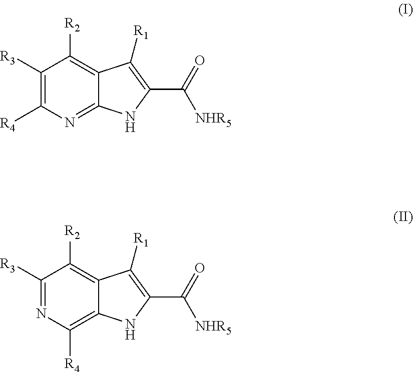

and to pharmaceutical compositions comprising the compounds. The compounds and compositions disclosed herein are antibacterials and are useful for the treatment of tuberculosis and other mycobacterial infections.

All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

Mycobacterium tuberculosis (“M. tb”) is the causative agent of tuberculosis (“TB”), a devastating infectious disease. It is estimated that about 2 million TB patients die each year globally. The treatment of drug-susceptible TB currently centers on four antibiotics, isoniazid, rifampicin, ethambutol, and pyrazinamide which were introduced more than 40 years ago (Franz 2017). Failure to properly treat tuberculosis has caused global drug resistance in Mtb and thus rendering some medications ineffective. A need exists in the art, therefore, to identify new chemical entities to treat TB.

### SUMMARY OF THE INVENTION

The present invention is directed to compounds of Formula (I) and Formula (II):

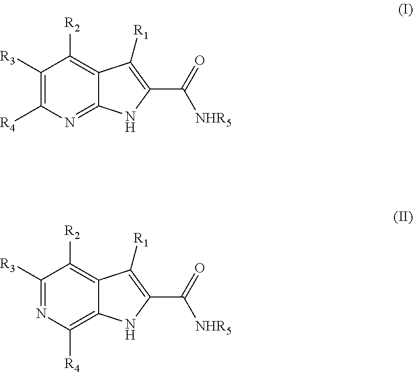

wherein:
R₁ is hydrogen or lower alkyl;
R₂ is hydrogen, lower alkyl, halo, cyano, trifluoromethyl, halo-lower alkyl, di-halo-lower alkyl, alkoxy, or carboxamide;
R₃ is hydrogen, lower alkyl, aryl, heteroaryl, halo, cyano, trifluoromethyl, halo-lower alkyl, di-halo-lower alkyl, alkoxy, or carboxamide;
R₄ is hydrogen, lower alkyl, aryl, heteroaryl, halo, cyano, trifluoromethyl, halo-lower alkyl, di-halo-lower alkyl, alkoxy, cycloalkoxy, or carboxamide;
R₅ is: lower alkyl, cycloalkyl, cycloalkylene or —CH₂-cycloalkyl, spiral(C₈-C₁₁)cycloalkyl, phenyl, a bridged cycloalkyl or

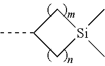

wherein m is 1, 2 or 3 and n is 1, 2, 3, or 4; or

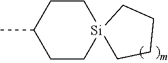

wherein m is 1 or 2;
or a pharmaceutically acceptable salt thereof.

The present invention is also directed to pharmaceutical compositions containing the above compounds and to methods of treating microbial infections such as tuberculosis.

### DETAILED DESCRIPTION OF THE INVENTION

It is to be understood that the terminology employed herein is for the purpose of describing particular embodiments, and is not intended to be limiting. Further, although any methods, devices and materials similar or equivalent to those described herein can be used in the practice or testing of the invention, certain methods, devices and materials are now described.

The present invention relates to novel azaindole carboxamide compounds, their preparations, and to their use as drugs for treating tuberculosis and other mycobacteria infections. The compounds, in certain embodiments, have the following general structures:

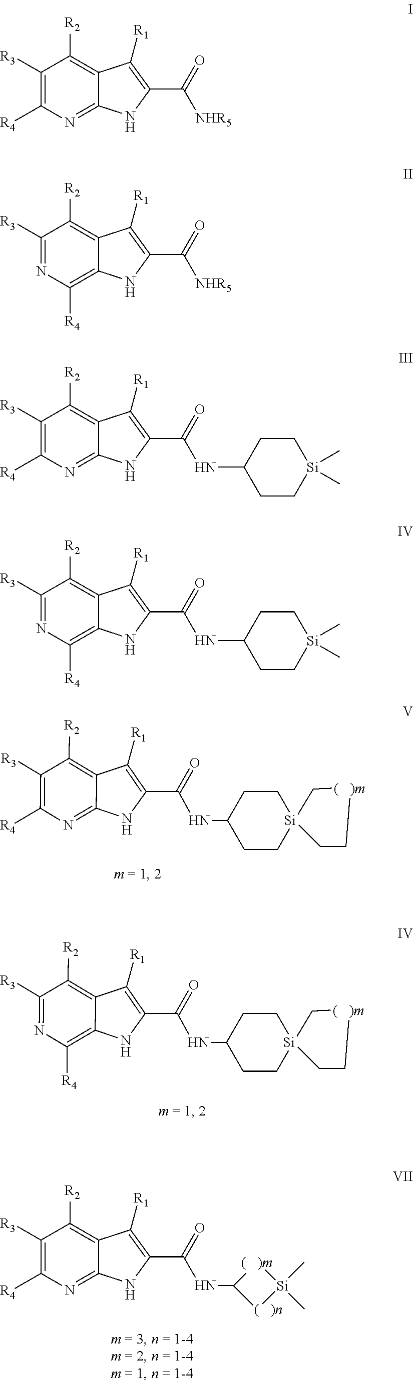

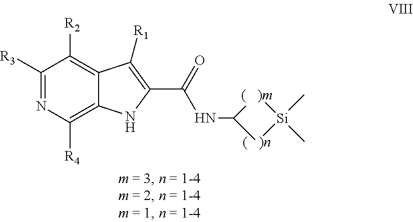

In one embodiment of the invention. the compounds of the invention can treat TB in combination with other anti-TB agents. The anti-TB agents include, but are not limited to, rifampicin, rifabutin, rifapentene, isoniazid, ethambutol, kanamycin, amikacin, capreomycin, clofazimine, cycloserine, para-aminosalicylic acid, linezolid, sutezolid, bedaquiline, delamanid, pretomanid, moxifloxacin, and levofloxacin.

### Definitions

As used herein, the term “alkyl”, alone or in combination with other groups, refers to a branched or straight-chain monovalent saturated aliphatic hydrocarbon radical of one to twenty carbon atoms, in one embodiment one to sixteen carbon atoms, in another embodiment one to ten carbon atoms.

As used herein, the term “alkenyl”, alone or in combination with other groups, refers to a straight-chain or branched hydrocarbon residue having an olefinic bond.

As used herein, the term “alkoxy” means alkyl-O—; and “alkoyl” means alkyl-CO—. Alkoxy substituent groups or alkoxy-containing substituent groups may be substituted by, for example, one or more alkyl or halo groups.

As used herein, the term “cycloalkoxy” means cycloalkyl-O—. Cycloalkoxy substituent groups may be substituted by, for example, one or more alkyl or halo groups.

As used herein, the term “halogen” means a fluorine, chlorine, bromine or iodine radical, in some embodiments a fluorine, chlorine or bromine radical.

The term “cycloalkyl” refers to a monovalent mono- or polycarbocyclic radical of three to ten, in one embodiment three to six carbon atoms. This term is further exemplified by radicals such as cyclopropyl, cyclobutyl, cyclopentyl, cyclohexyl, cycloheptyl, norbornyl, adamantyl, indanyl and the like. In one embodiment, the “cycloalkyl” moieties can optionally be substituted with one, two, three or four substituents. Each substituent can independently be alkyl, alkoxy, halogen, amino, hydroxyl or oxygen unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate.

The term “lower alkyl”, alone or in combination with other groups, refers to a branched or straight-chain alkyl radical of one to nine carbon atoms, in one embodiment one to six carbon atoms, in another embodiment one to four carbon atoms, in a further embodiment four to six carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, 1H-indenyl and the like.

The alkyl, lower alkyl and aryl groups may be substituted or unsubstituted. When substituted, there will generally be, for example, 1 to 4 substituents present. These substituents may optionally form a ring with the alkyl, lower alkyl or aryl group with which they are connected. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl, in other embodiments, for example, methoxy and ethoxy), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

As would be readily understood from the disclosure provided herein, any reference to a group falling within a generic group may be substituted or unsubstituted in the same manner. For example, a phenyl group may be substituted in the same manner as an aryl group.

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. Examples of such groups include, but not limited to, pyridinyl, pyrazinyl, pyridazinyl, 1,2,3-triazinyl, 1,2,4-triazinyl, oxazolyl, thiazolyl, and the like.

The heteroaryl group described above may be substituted independently with one, two, or three substituents. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl, benzothiazoyl and carbolinyl).

In some instances, a term is preceded by “(C_{#}-C_{#}).” As would be readily understood from the disclosure provided herein, this defines the number of carbon atoms associated with the term. For example, (C₁-C₆)alkyl means an alkyl in which the branched or straight-chain monovalent saturated aliphatic hydrocarbon radical has one to 6 carbon atoms. As would be readily understood from the disclosure provided herein, all substitution definitions apply equally to these structures. For example, (C₁-C₆)alkyl may be substituted in the same manner an alkyl is substituted.

By any range disclosed herein, it is meant that all integer unit amounts within the range are specifically disclosed as part of the invention. Thus, for example, 1 to 12 units means that 1, 2, 3 . . . 12 units are included as embodiments of this invention.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms.

In the practice of the method of the present invention, an effective amount of any one of the compounds of this invention, or a combination of any of the compounds of this invention, is administered via any of the usual and acceptable methods known in the art, either singly or in combination. The compounds or compositions can thus be administered, for example, ocularly, orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases. Thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are representative liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. In one embodiment, the therapeutically effective amount is in an amount of from about 10 mg to about 500 mg per day.

It will be appreciated that the compounds of the invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Compounds of the present invention can be prepared beginning with commercially available starting materials and utilizing general synthetic techniques and procedures known to those skilled in the art. Chemicals may be purchased from companies such, as for example, Aldrich, Argonaut Technologies, VWR and Lancaster. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography.

In some embodiments, R₅ is

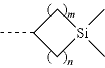

and m is 1-3 and n is 1-4. In another embodiment, m is 1 and n is 1. In another embodiment, m is 1 and n is 2. In another embodiment, m is 1 and n is 3. In another embodiment, m is 1 and n is 4. In another embodiment, m is 2 and n is 1. In another embodiment, m is 2 and n is 2. In another embodiment, m is 2 and n is 3. In another embodiment, m is 2 and n is 4. In another embodiment, m is 3 and n is 1. In another embodiment, m is 3 and n is 2. In another embodiment, m is 3 and n is 3. In another embodiment, m is 3 and n is 4. In the case where m is not equal to n, there exists a stereocenter in the amine and in the resulting amide. The product may be a mixture or it may be resolved individual stereoisomers of the amide although the absolute stereochemical assignments are not made. Under such a case, a number (MPL-xxx) without a suffix A or B is meant for a racemic mixture whereas suffix A and B (such as MPL-xxxA and MPL-xxxB) is meant to indicate resolved enantiomers although no absolute configuration has been assigned to each enantiomer. Separation of stereoisomers are most effectively achieved by the use of Super Fluid Chromatography (SFC) equipped with a chiral column.

### Synthesis of Representative Compounds of the Invention

The compounds of the invention can be prepared according to the following Scheme showing general methods A and B:

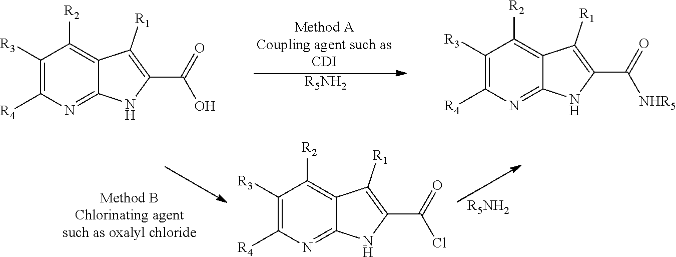

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Abbreviations used: ABPR, automatic back-pressure regulator; ACN, acetonitrile; aq., aqueous; 9-BBN, 9-borabicyclo[3.3.1]nonane; BINAP, 2,2′-bis(diphenylphosphino)-1,1′-binaphthyl; BMS, borane-dimethyl sulfide; Boc, tert-butoxycarbonyl;

CDI, 1,1′-carbonyl diimidazole; m-CPBA, meta-chloroperbenzoic acid; DABCO: 1,4-diazabicyclo[2.2.2]octane; DCM, dichloromethane; DEA, diethyl amine; DMAP, 4-dimethylaminopyridine; DME, dimethoxyethane; DMF, dimethylformamide; DMSO, dimethylsulfoxide; EDCI, 1-ethyl-3-(3-dimethylaminopropyl)carbodiimide; ESI, electrospray ionization; eq, equivalent; vHMDS, bis(trimethylsilyl)amine; NBS, N-bromosuccinimide; HOBt, hydroxybenzotriazole; HPLC, high performance liquid chromatography; IPA, isopropyl alcohol; LAH, lithium aluminium hydride; LCMS or LC-MS, liquid chromatography-mass spectrometry; LDA, lithium diisopropylamide; min, minute; m/z, mass-to-charge ratio; NCS, N-chlorosuccinimide; NFSI, N-fluorodibenzenesulfonimide; NIS, N-iodosuccinimide; nm, nanometer; NMP, N-methyl-2-pyrrolidone; NMI, 1-methylimidazole; NMR, nuclear magnetic resonance; ¹H NMR, proton NMR; Pd(dppf)Cl2, 1,1′-Bis(diphenylphosphino)ferrocene]dichloropalladium(II); Pd2(dpa)3, tris(dibenzylideneacetone)dipalladium(0); prep-HPLC, preparative HPLC; prep-TLC, preparative TLC; psi, pound per square inch; SFC, supercritical fluid chromatography; TBAF, tetra-n-butylammonium fluoride; TCFH, chloro-N,N,N′,N′-tetramethylformamidinium hexafluorophosphate; TEA, triethylamine; THF, tetrahydrofuran; TLC, Thin-layer chromatography; TIPS, triisopropyl silyl; TIPSCl, triisopropylsilyl chloride; TMEDA, tetramethylethylenediamine; TMS, trimethylsilyl; TMSCl, chloro(trimethyl)silane; Tos, p-tolylsulfonyl; TosCl, 4-Toluenesulfonyl chloride; ul, microliter; umol, micromole; XantPhos, 4,5-Bis(diphenylphosphino)-9,9-dimethylxanthene; XPhos, 2-dicyclohexylphosphino-2′,4′,6′-triisopropylbiphenyl; 8, chemical shift in ppm.

Reactions were monitored by TLC or LCMS and compounds were characterized by LCMS and/or NMR. Shimadzu LC20-MS2010 or LC20-MS2020 were used for LC/MS analysis. Varian 400 MHz, Varian 500 MHz or Bruker 500 MHz were used for NMR measurement.

General conditions for prep-HPLC purification: Instrument: Gilson GX281; Flow rate: 25 mL/min; Detector: UV 220 and UV 254.

“[water (X)-Y]; B %: J %-K %, L min” stands for mobile phase: A: X in water; B: Y; gradient J %-K % B over L min. For example, ‘[water (0.225% FA)-ACN]; B %: 36%-66%, 11 min’ means mobile phase: A: 0.025% formic acid in water, B: acetonitrile; gradient: 36%-66% B over 11 min.

### Example 1. MPL-015

### Synthesis of 4-(trifluoromethyl)-N-[(1S,2S,3S,5R)-2,6,6-trimethylnorpinan-3-yl]-1H-pyrrolo[2,3-b]pyridine-2-carboxamide

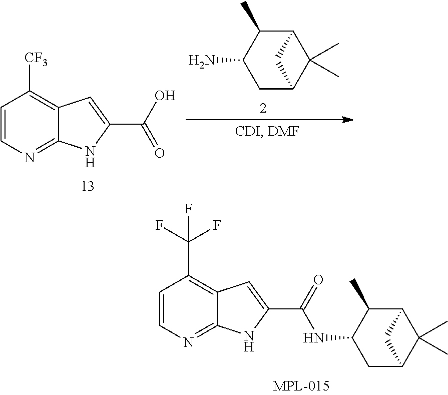 [SMILES: O=C(O)c1cc2c(C(F)(F)F)ccnc2[nH]1 ; C[C@@H]1[C@@H](N)C[C@H]2C[C@@H]1C2(C)C ; C[C@@H]1[C@@H](NC(=O)c2cc3c(C(F)(F)F)ccnc3[nH]2)C[C@H]2C[C@@H]1C2(C)C]

To a solution of 4-(trifluoromethyl)-1H-pyrrolo[2,3-b]pyridine-2-carboxylic acid (1.5 g, 6.52 mmol, 1 eq) in DMF (20 mL) was added CDI (1.59 g, 9.78 mmol, 1.5 eq) and stirred at 25° C. for 0.5 h. Then, (1S,2S,3S,5R)-2,6,6-trimethylnorpinan-3-amine (1.60 g, 10.43 mmol, 1.6 eq) was added above solution and stirred at 25° C. for 12 h. LCMS showed the starting material was consumed completely and one main peak with desired MS was detected. The mixture was added water (70 mL) and extracted with EtOAc (200 mL×3) and the organic phase was washed with water (30 mL×3) and brine (30 mL×3) and dried over Na₂SO₄ and filtered and concentrated under reduced pressure to give a residue. The residue was purified by column chromatography (SiO₂, DCM/MeOH=1/0 to 200/1). Compound 4-(trifluoromethyl)-N-[(1S,2S,3S,5R)-2,6,6-trimethylnorpinan-3-yl]-1H-pyrrolo[2,3-b]pyridine-2-carboxamide (2.07 g, 5.67 mmol, 86.92% yield, 100% purity) was obtained as a white solid.

LCMS (ESI) m/z: 366.2 [M+H]⁺

¹H NMR (500 MHz, DMSO-d₆) δ=12.78 (s, 1H), 8.63 (d, J=8.4 Hz, 1H), 8.56 (d, J=4.9 Hz, 1H), 7.50 (d, J=4.9 Hz, 1H), 7.40 (s, 1H), 4.47-4.35 (m, 1H), 2.47-2.35 (m, 2H), 2.10 (quin, J=7.1 Hz, 1H), 1.96 (br d, J=2.7 Hz, 1H), 1.83 (t, J=5.4 Hz, 1H), 1.86-1.80 (m, 1H), 1.73 (ddd, J=1.8, 6.4, 13.6 Hz, 1H), 1.26-1.20 (m, 4H), 1.11-1.05 (m, 6H).

### Example 2. MPL-016

### Synthesis of 4-methyl-1H-pyrrolo[2,3-b]pyridine-2-carbonyl chloride

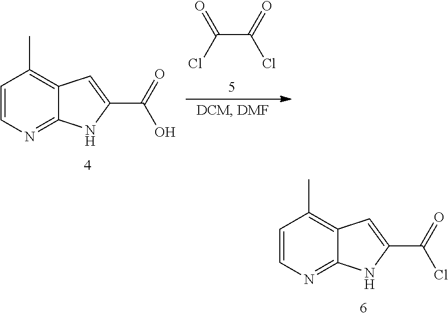

Oxalyl dichloride (1.62 g, 12.77 mmol, 1.12 mL, 15 eq) was added to the solution of 4-methyl-1H-pyrrolo[2,3-b]pyridine-2-carboxylic acid (150 mg, 851.44 umol, 1 eq) in DCM (8.0 mL). Then, 3 drops of DMF (3.11 mg, 42.57 umol, 3.28 uL, 0.05 eq) was added above solution and stirred at 25° C. for 2 hrs. LCMS showed the starting material consumed completely and the desired MS was detected. The residue was concentrated under reduced pressure to give a residue was added DCM (25 mL×3) and concentrated under reduced pressure to give a compound 4-methyl-1H-pyrrolo[2,3-b]pyridine-2-carbonyl chloride (160 mg, crude) as a yellow solid

LCMS (ESI) m/z: 190.9 [M+H]⁺;

### Synthesis of 4-methyl-N-[(1S,2S,3S,5R)-2,6,6-trimethylnorpinan-3-yl]-1H-pyrrolo[2,3-b]pyridine-2-carboxamide

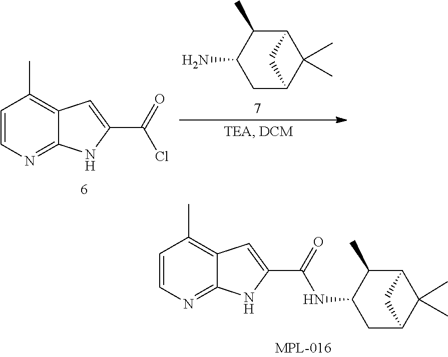 [SMILES: Cc1ccnc2[nH]c(C(=O)Cl)cc12 ; C[C@@H]1[C@@H](N)C[C@H]2C[C@@H]1C2(C)C ; Cc1ccnc2[nH]c(C(=O)N[C@H]3C[C@H]4C[C@@H]([C@@H]3C)C4(C)C)cc12]

4-Methyl-1H-pyrrolo[2,3-b]pyridine-2-carbonyl chloride (160 mg, 822.13 umol, 0.91 eq) was added to the solution of (1S,2S,3S,5R)-2,6,6-trimethylnorpinan-3-amine (138.46 mg, 903.44 umol, 1 eq) and TEA (365.68 mg, 3.61 mmol, 502.99 uL, 4.0 eq) in DCM (10 mL) and stirred at 25° C. for 2.0 hrs. LCMS showed the starting material was consumed completely and the desired MS was detected. The mixture was added water (20 mL) and extracted with DCM (30 mL×3). The organic phase was dried over Na₂SO₄, filtered and concentrated under reduced pressure to give a residue. The residue was purified by column chromatography (SiO₂, DCM:MeOH=1/0 to 14:1) Compound 4-methyl-N-[(1S,2S,3S,5R)-2,6,6-trimethylnorpinan-3-yl]-1H-pyrrolo[2,3-b]pyridine-2-carboxamide (15 mg, 48.17 umol, 5.33% yield, 100% purity) was obtained as yellow solid. LCMS (ESI) m/z: 312.2 [M+H]⁺;

1H NMR (400 MHz, DMSO-d6) δ=11.96 (br s, 1H), 8.33 (br d, J=8.4 Hz, 1H), 8.17 (d, J=4.6 Hz, 1H), 7.21 (s, 1H), 6.91 (d, J=4.6 Hz, 1H), 4.43-4.25 (m, 1H), 2.51 (s, 3H), 2.45-2.29 (m, 2H), 2.05 (quin, J=7.0 Hz, 1H), 1.93 (br s, 1H), 1.80 (br t, J=5.2 Hz, 1H), 1.71-1.63 (m, 1H), 1.21 (s, 3H), 1.17 (br d, J=9.5 Hz, 1H), 1.07-1.00 (m, 6H).

### Example 3. MPL-017

### Synthesis of 4-cyclopropyl-1H-pyrrolo[2,3-b]pyridine-2-carbonyl chloride

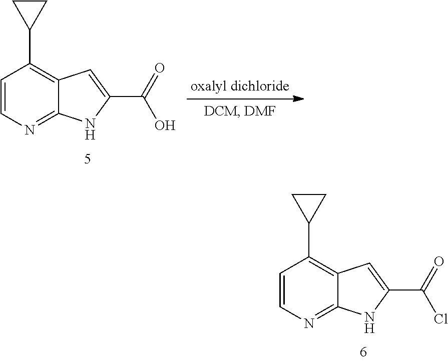 [SMILES: O=C(O)c1cc2c(C3CC3)ccnc2[nH]1 ; O=C(Cl)c1cc2c(C3CC3)ccnc2[nH]1]

Oxalyl dichloride (3.77 g, 29.67 mmol, 2.60 mL, 40 eq) was added to the solution of 4-cyclopropyl-1H-pyrrolo[2,3-b]pyridine-2-carboxylic acid (150 mg, 741.81 umol, 1 eq) in DCM (10 mL). Then, 3 drops of DMF (2.71 mg, 37.09 umol, 2.85 uL, 0.05 eq) was added above solution and stirred at 25° C. for 2.0 hrs. LCMS showed the starting material was consumed completely and the desired MS was detected. The residue was concentrated under reduced pressure to give a residue was added DCM (25 mL×3) and concentrated under reduced pressure to give a compound 4-cyclopropyl-1H-pyrrolo[2,3-b]pyridine-2-carbonyl chloride (160 mg, crude) as a yellow solid. LCMS (ESI) m/z: 217 [M+H]⁺;

### Synthesis of 4-cyclopropyl-N-(4,4-dimethylcyclohexyl)-1H-pyrrolo[2,3-b]pyridine-2-carboxamide

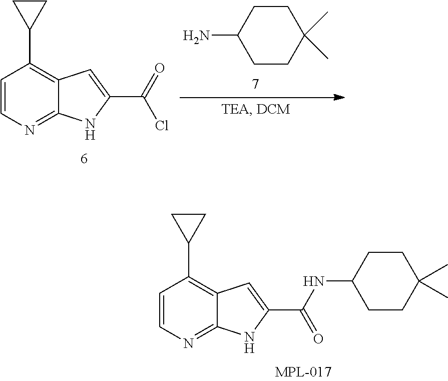

4,4-Dimethylcyclohexanamine (90 mg, 707.40 umol, 1 eq) was added to the solution of 4-cyclopropyl-1H-pyrrolo[2,3-b]pyridine-2-carbonyl chloride (160 mg, 725.12 umol, 1.03 eq) in DCM (10 mL). Then, TEA (214.75 mg, 2.12 mmol, 295.39 uL, 3.0 eq) was added above solution and stirred at 25° C. for 12 hrs. LCMS showed the desired MS was detected. The mixture was added water (15 mL) and extracted with DCM (50 mL×3) and the organic phase was dried over Na₂SO₄ and filtered and concentrated under reduced pressure to give a residue. The residue was purified by column chromatography (SiO₂, DCM:MeOH=1/0 to 50:1).

Compound 4-cyclopropyl-N-(4,4-dimethylcyclohexyl)-1H-pyrrolo[2,3-b]pyridine-2-carboxamide (44.4 mg, 139.84 umol, 19.77% yield, 98.086% purity) was obtained as a white solid. LCMS (ESI) m/z: 312.2 [M+H]⁺;

¹H NMR (400 MHz, DMSO-d6) δ=13.01 (br s, 1H), 8.61 (br d, J=7.9 Hz, 1H), 8.25 (d, J=5.7 Hz, 1H), 7.55 (s, 1H), 6.94 (d, J=6.0 Hz, 1H), 3.78-3.64 (m, 1H), 2.44-2.35 (m, 1H), 1.71-1.63 (m, 2H), 1.59-1.48 (m, 2H), 1.44-1.36 (m, 2H), 1.36-1.24 (m, 4H), 1.18-1.13 (m, 2H), 0.93 (s, 3H), 0.91 (s, 3H).

### Example 4. MPL-019

### Synthesis of 4,6-dimethyl-1H-pyrrolo[2,3-b]pyridine-2-carbonyl chloride

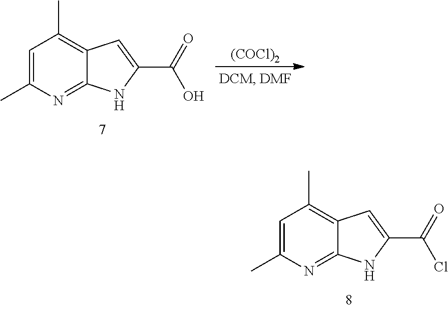

Oxalyl dichloride (8.70 g, 68.54 mmol, 6.0 mL, 128.28 eq) was added to the solution of 4,6-dimethyl-1H-pyrrolo[2,3-b]pyridine-2-carboxylic acid (100 mg, 525.77 umol, 1 eq) in DCM (6.0 mL). Then, 3 drops of DMF (1.92 mg, 26.29 umol, 2.02 uL, 0.05 eq) was added above solution and stirred at 25° C. for 1.5 hrs. LCMS showed the starting material was consumed completely and the desired MS was detected. The residue was concentrated under reduced pressure to give a residue was added DCM (25 mL×3) and concentrated under reduced pressure to give a compound 4,6-dimethyl-1H-pyrrolo[2,3-b]pyridine-2-carbonyl chloride (100 mg, crude) as a yellow solid. LCMS (ESI) m/z: 205.1 [M+H]⁺;

### Synthesis of N-(4,4-dimethylcyclohexyl)-4,6-dimethyl-1H-pyrrolo[2,3-b]pyridine-2-carboxamide

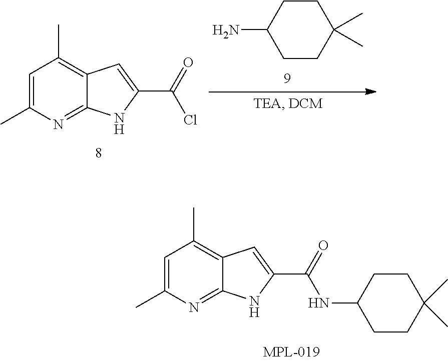

To a solution of 4,6-dimethyl-1H-pyrrolo[2,3-b]pyridine-2-carbonyl chloride (100 mg, 479.29 umol, 1 eq) and 4,4-dimethylcyclohexanamine (121.96 mg, 958.57 umol, 2.0 eq) in DCM (10 mL) was added TEA (145.50 mg, 1.44 mmol, 200.13 uL, 3.0 eq). The mixture was stirred at 25° C. for 0.5 hr. LCMS showed the starting material was consumed completely and the desired MS was detected. The mixture was concentrated under reduced pressure to give a residue. The residue was purified by column chromatography (SiO₂, DCM:MeOH=1/0 to 150:1).

Compound N-(4,4-dimethylcyclohexyl)-4,6-dimethyl-1H-pyrrolo[2,3-b]pyridine-2-carboxamide (64.7 mg, 186.68 umol, 38.95% yield, 99.671% purity, FA) was obtained as a white solid. LCMS (ESI) m/z: 300.2 [M+H]⁺;

¹H NMR (400 MHz, DMSO-d6) δ=11.57 (br s, 1H), 8.25 (d, J=7.9 Hz, 1H), 8.12 (s, 1H), 7.10 (s, 1H), 6.85 (s, 1H), 3.81-3.66 (m, 1H), 2.47-2.46 (m, 3H), 2.45 (s, 3H), 1.70-1.63 (m, 2H), 1.59-1.47 (m, 2H), 1.43-1.36 (m, 2H), 1.32-1.23 (m, 2H), 0.92 (d, J=10.4 Hz, 6H)

### Example 5. MPL-022

### Synthesis of 4-cyclopropyl-N-[(1S,2S,3S,5R)-2,6,6-trimethylnorpinan-3-yl]-1H-pyrrolo[2,3-b]pyridine-2-carboxamide

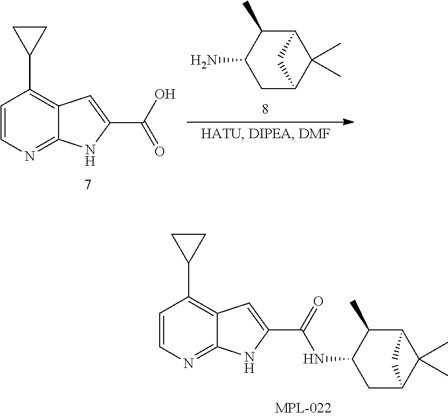 [SMILES: O=C(O)c1cc2c(C3CC3)ccnc2[nH]1 ; C[C@@H]1[C@@H](N)C[C@H]2C[C@@H]1C2(C)C ; C[C@@H]1[C@@H](NC(=O)c2cc3c(C4CC4)ccnc3[nH]2)C[C@H]2C[C@@H]1C2(C)C]

To a solution of 4-cyclopropyl-1H-pyrrolo[2,3-b]pyridine-2-carboxylic acid (200 mg, 989.08 umol, 1 eq) in DMF (10 mL) was added HATU (451.29 mg, 1.19 mmol, 1.2 eq), DIEA (383.49 mg, 2.97 mmol, 516.84 uL, 3.0 eq) and (1S,2S,3S,5R)-2,6,6-trimethylnorpinan-3-amine (166.75 mg, 1.09 mmol, 1.1 eq). The reaction was stirred at 25° C. for 3.0 hrs. LCMS showed the starting material was consumed completely and the desired MS was detected. The mixture was added to water (40 mL), extracted with EtOAc (50×3 mL). The organic phase was washed with brine (20 mL×2), dried over Na₂SO₄, filtered and concentrated under reduced pressure to give a residue. The residue was purified by prep-HPLC (column: Phenomenex Synergi C18 150*30 mm*4 um; mobile phase: [water (0.225% FA)-ACN]; B %: 36%-66%, 11 min). Compound 4-cyclopropyl-N-[(1S,2S,3S,5R)-2,6,6-trimethylnorpinan-3-yl]-1H-pyrrolo[2,3-b]pyridine-2-carboxamide (16 mg, 47.41 umol, 4.79% yield, 100% purity) was obtained as a light brown solid. LCMS (ESI) m/z: 338.2 [M+H]⁺; ¹H NMR (400 MHz, DMSO-d6) δ=12.03 (br s, 1H), 8.34 (d, J=8.4 Hz, 1H), 8.14 (d, J=5.1 Hz, 1H), 7.31 (s, 1H), 6.68 (d, J=5.1 Hz, 1H), 4.36 (quin, J=8.0 Hz, 1H), 2.46-2.33 (m, 2H), 2.29-2.21 (m, 1H), 2.10-2.00 (m, 1H), 1.94 (br s, 1H), 1.83-1.78 (m, 1H), 1.72-1.63 (m, 1H), 1.22 (s, 3H), 1.19-1.11 (m, 3H), 1.05 (t, J=3.5 Hz, 6H), 1.01-0.95 (m, 2H).

### Example 6. MPL-029

### Synthesis of 4-methyl-N-(4-methylcyclohexyl)-1H-pyrrolo[2,3-b]pyridine-2-carboxamide

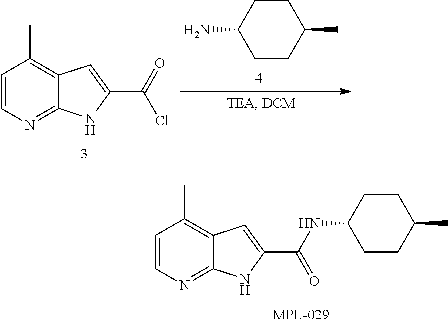

4-Methylcyclohexanamine (70 mg, 618.37 umol, 1 eq) was added to the solution of 4-methyl-1H-pyrrolo[2,3-b]pyridine-2-carbonyl chloride (110 mg, 565.21 umol, 9.14e-1 eq) in DCM (8.0 mL). Then, TEA (187.72 mg, 1.86 mmol, 258.21 uL, 3.0 eq) was added above solution and stirred at 25° C. for 2.0 hrs. LCMS showed the starting material was consumed completely. The mixture was added water (15 mL) and extracted with DCM (50 mL×3). The organic phase was dried over Na₂SO₄, filtered and concentrated under reduced pressure to give a residue. The residue was purified by column chromatography (SiO₂, DCM:MeOH=1/0 to 50:1). Compound 4-methyl-N-(4-methylcyclohexyl)-1H-pyrrolo[2,3-b]pyridine-2-carboxamide (57 mg, 210.06 umol, 33.97% yield, 100% purity) was obtained as a yellow solid.

LCMS (ESI) m/z: 272.2 [M+H]⁺; ¹H NMR (400 MHz, DMSO-d6) δ=12.65 (br s, 1H), 8.46 (br d, J=8.2 Hz, 1H), 8.27 (br s, 1H), 7.36 (s, 1H), 7.14 (br d, J=4.0 Hz, 1H), 3.80-3.60 (m, 1H), 2.60 (s, 3H), 1.84 (br d, J=10.6 Hz, 2H), 1.69 (br d, J=12.3 Hz, 2H), 1.41-1.27 (m, 3H), 1.08-0.94 (m, 2H), 0.87 (d, J=6.4 Hz, 3H).

### Example 7. MPL-031

### Synthesis of N-cyclooctyl-4-methyl-1H-pyrrolo[2,3-b]pyridine-2-carboxamide

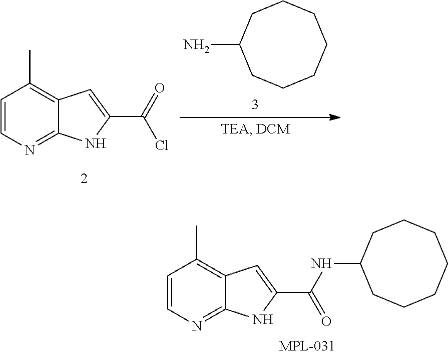 [SMILES: Cc1ccnc2[nH]c(C(=O)Cl)cc12 ; NC1CCCCCCC1 ; Cc1ccnc2[nH]c(C(=O)NC3CCCCCCC3)cc12]

4-Methyl-1H-pyrrolo[2,3-b]pyridine-2-carbonyl chloride (130 mg, 667.98 umol, 0.91 eq) was added to the solution of cyclooctanamine (100 mg, 786.00 umol, 1.07 eq) and TEA (222.83 mg, 2.20 mmol, 306.51 uL, 3.0 eq) in DCM (8.0 mL) and stirred at 25° C. for 2.0 hrs. LCMS showed the starting material was consumed completely and the desired MS was detected. The mixture was concentrated under reduced pressure to give a residue. The residue was purified by column chromatography (SiO₂, DCM:MeOH=1/0 to 90:1). Then, the residue was purified by prep-HPLC (column: Phenomenex Synergi C18 150*30 mm*4 um; mobile phase: [water (0.225% FA)-ACN]; B %: 35%-55%, 11 min). Compound N-cyclooctyl-4-methyl-1H-pyrrolo[2,3-b]pyridine-2-carboxamide (27.3 mg, 95.66 umol, 13.03% yield, 100% purity) was obtained as a light brown solid.

LCMS (ESI) m/z: 286.2 [M+H]⁺; ¹H NMR (400 MHz, DMSO-d6) δ=11.99 (br s, 1H), 8.28-8.10 (m, 2H), 7.22 (s, 1H), 6.94 (br s, 1H), 4.09-3.96 (m, 1H), 2.52 (br s, 3H), 1.83-1.66 (m, 6H), 1.62-1.47 (m, 8H).

### Example 8. MPL-010

### Synthesis of N-(4,4-dimethylcyclohexyl)-4-(trifluoromethyl)-1H-pyrrolo[2,3-b]pyridine-2-carboxamide

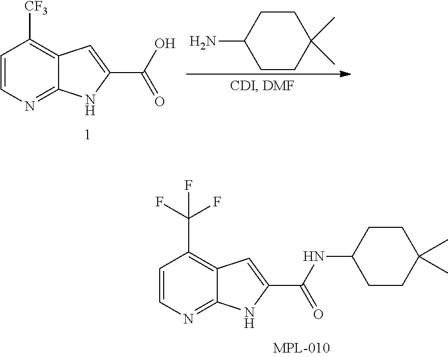

To a solution of 4-(trifluoromethyl)-1H-pyrrolo[2,3-b]pyridine-2-carboxylic acid (80 mg, 347.61 umol, 1 eq) in DMF (5.0 mL) was added CDI (73.27 mg, 451.89 umol, 1.3 eq) and stirred at 25° C. for 15 min. Then, 4,4-dimethylcyclohexanamine (66.34 mg, 521.41 umol, 1.5 eq) was added above solution and stirred at 25° C. for 12 hrs. LCMS showed the starting material was consumed completely and one main peak with desired MS was detected. The mixture was diluted with DCM (20 mL) and washed with water (20 mL×5) and HCl (1M, 20 mL). The organic layers were dried over anhydrous Na₂SO₄ and concentrated under reduced pressure to give a residue. The residue was purified by column chromatography (SiO₂, DCM:MeOH=1/0 to 80:1). Compound N-(4,4-dimethylcyclohexyl)-4-(trifluoromethyl)-1H-pyrrolo[2,3-b]pyridine-2-carboxamide (30.1 mg, 87.99 umol, 25.31% yield, 99.203% purity) was obtained as a white solid.

LCMS (ESI) m/z: 340.1 [M+H]⁺;

¹H NMR (400 MHz, DMSO-d6) δ=12.71 (br s, 1H), 8.56-8.42 (m, 2H), 7.46 (d, J=4.9 Hz, 1H), 7.33 (d, J=1.2 Hz, 1H), 3.82-3.62 (m, 1H), 1.70-1.61 (m, 2H), 1.59-1.45 (m, 2H), 1.43-1.34 (m, 2H), 1.33-1.21 (m, 2H), 0.92 (d, J=9.3 Hz, 6H).

### Example 9. MPL-013

### Synthesis of 4-cyano-N-[(1S,2S,3S,5R)-2,6,6-trimethylnorpinan-3-yl]-1H-pyrrolo[2,3-b]pyridine-2-carboxamide

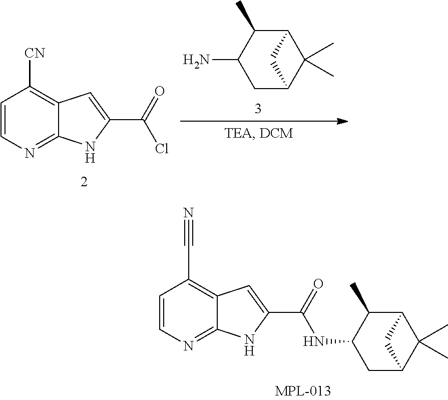 [SMILES: N#Cc1ccnc2[nH]c(C(=O)Cl)cc12 ; C[C@@H]1C(N)C[C@H]2C[C@@H]1C2(C)C ; C[C@@H]1[C@@H](NC(=O)c2cc3c(C#N)ccnc3[nH]2)C[C@H]2C[C@@H]1C2(C)C]

To a solution of 4-cyano-1H-pyrrolo[2,3-b]pyridine-2-carbonyl chloride (100 mg, 486.38 umol, 1.0 eq) and (1S,2S,3S,5R)-2,6,6-trimethylnorpinan-3-amine (149.09 mg, 972.76 umol, 2.0 eq) in DCM (10 mL) was added TEA (147.65 mg, 1.46 mmol, 203.10 uL, 3.0 eq). The mixture was stirred at 25° C. for 0.5 hr. LCMS showed the starting material was consumed completely and the desired mass was detected. The mixture was concentrated under reduced pressure to give a residue. The residue was purified by column chromatography (SiO₂, DCM:MeOH=1/0 to 200:1). Compound 4-cyano-N-[(1S,2S,3S,5R)-2,6,6-trimethylnorpinan-3-yl]-1H-pyrrolo[2,3-b]pyridine-2-carboxamide (87.3 mg, 270.78 umol, 55.67% yield, 100% purity) was obtained as a white solid.

LCMS (ESI) m/z: 323.2 [M+H]⁺; ¹H NMR (400 MHz, DMSO-d6) δ=12.86 (s, 1H), 8.63 (br d, J=8.6 Hz, 1H), 8.52 (d, J=4.9 Hz, 1H), 7.64 (d, J=4.9 Hz, 1H), 7.48 (s, 1H), 4.46-4.32 (m, 1H), 2.47-2.32 (m, 2H), 2.09 (quin, J=7.4 Hz, 1H), 1.99-1.92 (m, 1H), 1.85-1.79 (m, 1H), 1.76-1.67 (m, 1H), 1.24 (s, 3H), 1.20 (d, J=9.5 Hz, 1H), 1.07 (t, J=3.5 Hz, 6H).

### Example 10. MPL-024

### Synthesis of 4,6-dimethyl-N-[(1S,2S,3S,5R)-2,6,6-trimethylnorpinan-3-yl]-1H-pyrrolo[2,3-b]pyridine-2-carboxamide

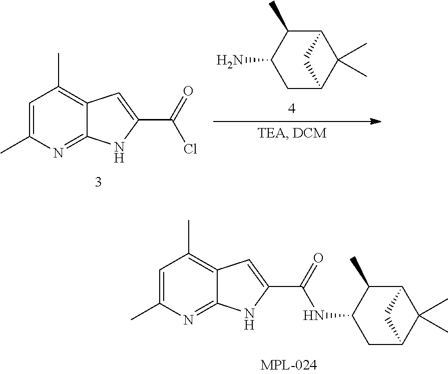

To a solution of 4,6-dimethyl-1H-pyrrolo[2,3-b]pyridine-2-carbonyl chloride (100 mg, 479.29 umol, 1 eq) and (1S,2S,3S,5R)-2,6,6-trimethylnorpinan-3-amine (146.91 mg, 958.57 umol, 2.0 eq) in DCM (10 mL) was added TEA (145.50 mg, 1.44 mmol, 200.13 uL, 3.0 eq). The mixture was stirred at 25° C. for 1.0 hr. LCMS showed the starting material was consumed completely and the desired mass was detected. The mixture was concentrated under reduced pressure to give a residue. The residue was purified by column chromatography (SiO₂, DCM:MeOH=1/0 to 170:1). Compound 4,6-dimethyl-N-[(1S,2S,3S,5R)-2,6,6-trimethylnorpinan-3-yl]-1H-pyrrolo[2,3-b]pyridine-2-carboxamide (84 mg, 257.77 umol, 53.78% yield, 99.87% purity) was obtained as a white solid.

LCMS (ESI) m/z: 326.2 [M+H]⁺; ¹H NMR (400 MHz, DMSO-d6) δ=11.55 (s, 1H), 11.62-11.50 (m, 1H), 8.36 (br d, J=8.6 Hz, 1H), 7.13 (d, J=2.0 Hz, 1H), 6.85 (s, 1H), 4.48-4.28 (m, 1H), 2.47-2.46 (m, 3H), 2.45 (s, 3H), 2.43-2.26 (m, 2H), 2.12-2.02 (m, 1H), 1.93 (br s, 1H), 1.80 (br t, J=5.2 Hz, 1H), 1.75-1.65 (m, 1H), 1.24-1.19 (m, 4H), 1.08-1.02 (m, 6H).

### Example 11. MPL-036

### Synthesis of 4-cyano-N-cyclooctyl-1H-pyrrolo[2,3-b]pyridine-2-carboxamide

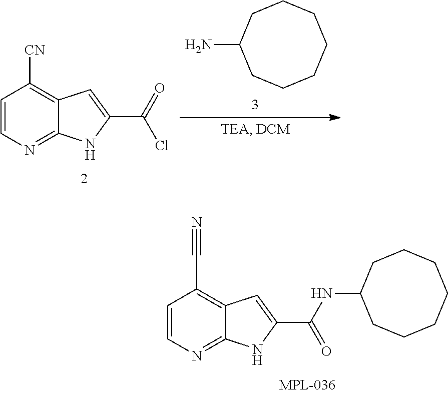 [SMILES: N#Cc1ccnc2[nH]c(C(=O)Cl)cc12 ; NC1CCCCCCC1 ; N#Cc1ccnc2[nH]c(C(=O)NC3CCCCCCC3)cc12]

To a solution of 4-cyano-1H-pyrrolo[2,3-b]pyridine-2-carbonyl chloride (100 mg, 486.38 umol, 1.0 eq) and cyclooctanamine (123.76 mg, 972.76 umol, 2.0 eq) in DCM (10 mL) was added TEA (147.65 mg, 1.46 mmol, 203.10 uL, 3.0 eq). The mixture was stirred at 25° C. for 0.5 hr. LCMS showed the starting material was consumed completely and the desired MS was detected. The mixture was concentrated under reduced pressure to give a residue. The residue was purified by prep-HPLC (column: DAICEL CHIRALPAK AS-H (250 mm*30 mm, 5 um); mobile phase: [0.1% NH3H2O ETOH]; B %: 25%-25%, min) from SFC. Compound 4-cyano-N-cyclooctyl-1H-pyrrolo[2,3-b]pyridine-2-carboxamide (10 mg, 33.74 umol, 6.94% yield, 100% purity) was obtained as a white solid.

LCMS (ESI) m/z: 297.2 [M+H]⁺; 1H NMR (400 MHz, DMSO-d6) δ=12.80 (br s, 1H), 8.55-8.43 (m, 2H), 7.60 (br d, J=4.6 Hz, 1H), 7.42 (s, 1H), 4.03 (br s, 1H), 1.81-1.43 (m, 14H).

### Example 12. MPL-037

### Synthesis of N-cyclooctyl-4-(trifluoromethyl)-1H-pyrrolo[2,3-b]pyridine-2-carboxamide

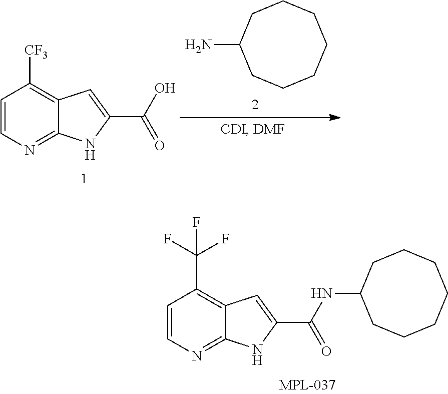 [SMILES: O=C(O)c1cc2c(C(F)(F)F)ccnc2[nH]1 ; NC1CCCCCCC1 ; O=C(NC1CCCCCCC1)c1cc2c(C(F)(F)F)ccnc2[nH]1]

To a solution of 4-(trifluoromethyl)-1H-pyrrolo[2,3-b]pyridine-2-carboxylic acid (200 mg, 869.02 umol, 1 eq) in DMF (4.5 mL) was added CDI (183.18 mg, 1.13 mmol, 1.3 eq) and stirred at 25° C. for 15 min. Then, cyclooctanamine (176.90 mg, 1.39 mmol, 1.6 eq) was added above solution and stirred at 25° C. for 12 h. LCMS showed one main peak with desired MS was detected. The mixture was added water (10 mL) and extracted with EtOAc (15 mL×3) and the organic phase was washed with water (10 mL×3) and brine (10 mL×3) and dried over Na₂SO₄ and filtered and concentrated under reduced pressure to give a residue. The residue was purified by column chromatography (SiO₂, DCM/MeOH=1/0 to 160:1). Compound N-cyclooctyl-4-(trifluoromethyl)-1H-pyrrolo[2,3-b]pyridine-2-carboxamide (212.3 mg, 622.08 umol, 71.58% yield, 99.437% purity) was obtained as a white solid.

LCMS (ESI) m/z: 340.1 [M+H]⁺; ¹H NMR (500 MHz, DMSO-d₆) δ=12.73 (br s, 1H), 8.62-8.44 (m, 2H), 7.47 (d, J=4.7 Hz, 1H), 7.37 (s, 1H), 4.06 (br dd, J=3.8, 8.1 Hz, 1H), 1.85-1.64 (m, 6H), 1.63-1.46 (m, 8H).

### Example 13 MPL-039

### Synthesis of N-cyclooctyl-4-cyclopropyl-1H-pyrrolo[2,3-b]pyridine-2-carboxamide

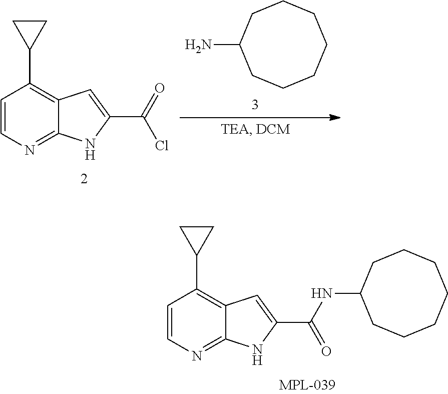 [SMILES: O=C(Cl)c1cc2c(C3CC3)ccnc2[nH]1 ; NC1CCCCCCC1 ; O=C(NC1CCCCCCC1)c1cc2c(C3CC3)ccnc2[nH]1]

Cyclooctanamine (100 mg, 786.00 umol, 1 eq) was added to the solution of 4-cyclopropyl-1H-pyrrolo[2,3-b]pyridine-2-carbonyl chloride (160 mg, 725.12 umol, 9.23e⁻¹ eq) in DCM (10 mL). Then, TEA (238.61 mg, 2.36 mmol, 328.21 uL, 3.0 eq) was added above solution and stirred at 25° C. for 2.0 hrs. LCMS showed the starting material was consumed completely and the desired MS was detected. The mixture was concentrated under reduced pressure to give a residue. The residue was purified by column chromatography (SiO₂, DCM:MeOH=1/0 to 80:1). Compound N-cyclooctyl-4-cyclopropyl-1H-pyrrolo[2,3-b]pyridine-2-carboxamide (106.1 mg, 333.83 umol, 42.47% yield, 97.984% purity) was obtained as a light brown solid.

LCMS (ESI) m/z: 312.2 [M+H]⁺; ¹H NMR (400 MHz, DMSO-d6) δ=13.10 (br s, 1H), 8.66 (br d, J=7.9 Hz, 1H), 8.26 (d, J=6.0 Hz, 1H), 7.59 (s, 1H), 6.95 (d, J=6.0 Hz, 1H), 4.09-3.94 (m, 1H), 2.45-2.37 (m, 1H), 1.81-1.62 (m, 6H), 1.52 (br t, J=10.9 Hz, 8H), 1.38-1.28 (m, 2H), 1.20-1.13 (m, 2H).

### Example 14. MPL-041

### Synthesis of 4,6-dimethyl-1H-pyrrolo[2,3-b]pyridine-2-carbonyl chloride

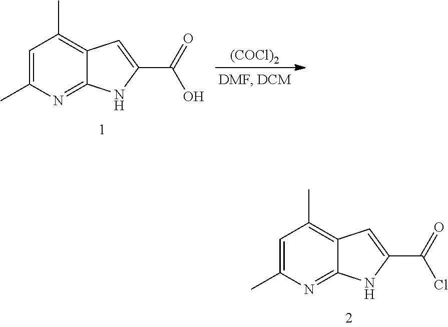

Oxalyl dichloride (8.70 g, 68.54 mmol, 6.0 mL, 128.28 eq) was added to the solution of 4,6-dimethyl-1H-pyrrolo[2,3-b]pyridine-2-carboxylic acid (100 mg, 525.77 umol, 1 eq) in DCM (6.0 mL). Then, 3 drops of DMF (1.92 mg, 26.29 umol, 2.02 uL, 0.05 eq) was added above solution and stirred at 25° C. for 1.5 hrs. LCMS showed the starting material was consumed completely and the desired mass was detected. The residue was concentrated under reduced pressure to give a residue was added DCM (25 mL 3 3) and concentrated under reduced pressure to give a compound 4,6-dimethyl-1H-pyrrolo[2,3-b]pyridine-2-carbonyl chloride (100 mg, crude) as a yellow solid. LCMS (ESI) m/z: 205.0 [M+H]⁺;

### Synthesis of N-cyclooctyl-4,6-dimethyl-1H-pyrrolo[2,3-b]pyridine-2-carboxamide

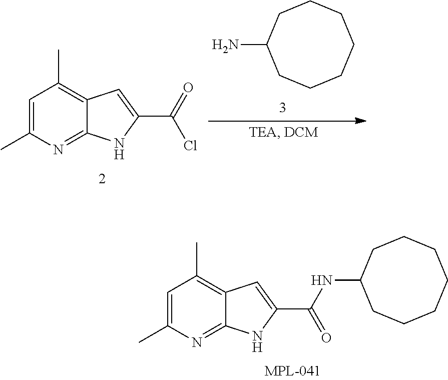 [SMILES: Cc1cc(C)c2cc(C(=O)Cl)[nH]c2n1 ; NC1CCCCCCC1 ; Cc1cc(C)c2cc(C(=O)NC3CCCCCCC3)[nH]c2n1]

To a solution of 4,6-dimethyl-1H-pyrrolo[2,3-b]pyridine-2-carbonyl chloride (100 mg, 479.29 umol, 1 eq) and cyclooctanamine (121.96 mg, 958.57 umol, 2.0 eq) in DCM (10. mL) was added TEA (145.50 mg, 1.44 mmol, 200.13 uL, 3.0 eq). The mixture was stirred at 25° C. for 0.5 hr. LCMS showed the starting material was consumed completely and the desired MS was detected. The mixture was concentrated under reduced pressure to give a residue. The residue was purified by column chromatography (SiO₂, DCM:MeOH=1/0 to 150:1). Compound N-cyclooctyl-4,6-dimethyl-1H-pyrrolo[2,3-b]pyridine-2-carboxamide (114.7 mg, 330.72 umol, 69.00% yield, 99.602% purity, FA) was obtained as a white solid.

LCMS (ESI) m/z: 300.2 [M+H]⁺; ¹H NMR (400 MHz, DMSO-d6) δ=11.54 (br s, 1H), 8.27 (d, J=7.9 Hz, 1H), 8.13 (s, 1H), 7.11 (s, 1H), 6.85 (s, 1H), 4.10-3.95 (m, 1H), 2.47-2.46 (m, 3H), 2.45 (s, 3H), 1.80-1.47 (m, 14H).

### Example 15. MPL-068

### Scheme

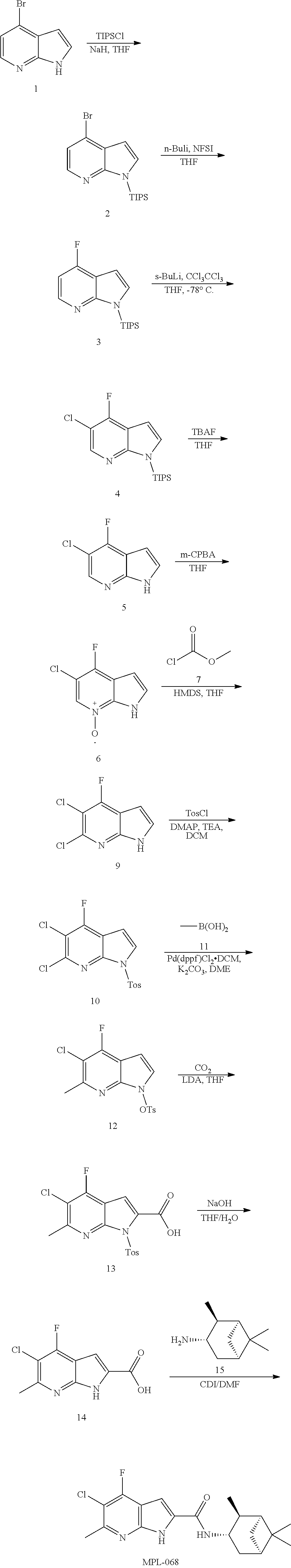

### Synthesis of 5-chloro-4-fluoro-7-oxido-1H-pyrrolo[2,3-b]pyridin-7-ium

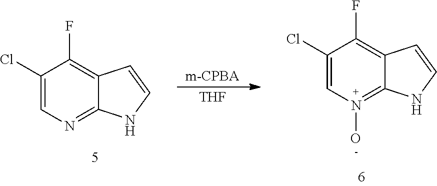

To a solution of 5-chloro-4-fluoro-1H-pyrrolo[2,3-b]pyridine (4.4 g, 25.80 mmol, 1 eq) in DCM (50 mL) was added m-CPBA (12.94 g, 63.74 mmol, 85% purity, 2.47 eq) at 0° C. The mixture was stirred at 30° C. for 12 hr. LCMS showed the reactant 5 was consumed completely. The sat. Na₂SO₃ (100 ml) was added to the mixture and the reaction mixture was stirred for 0.5 h. Then filtered and the inorganic phase was extracted with DCM (100 mL×3). The combined organic layers were dried over anhydrous Na₂SO₄, filtered and concentrated under reduced pressure to give a residue. The crude product was used directly for the next step without purification. The crude product 5-chloro-4-fluoro-7-oxido-1H-pyrrolo[2,3-b]pyridin-7-ium (12 g, 22.51 mmol, 87.27% yield, 35% purity) was obtained as brown solid.

### Synthesis of 5,6-dichloro-4-fluoro-1H-pyrrolo[2,3-b]pyridine

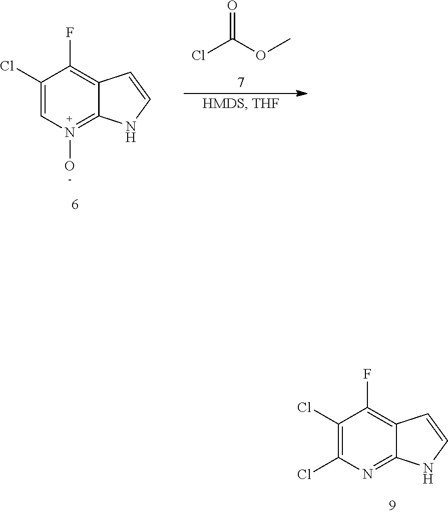

To a solution of 5-chloro-4-fluoro-7-oxido-1H-pyrrolo[2,3-b]pyridin-7-ium (11 g, 20.64 mmol, 1 eq) and HMDS (3.33 g, 20.64 mmol, 4.33 mL, 1 eq) in THE (110 mL) was added dropwise methyl carbonochloridate (4.88 g, 51.59 mmol, 4.00 mL, 2.5 eq) under N₂, the mixture was stirred at 30° C. for 24 h. LC-MS showed the desired MS was detected. The solvent was removed under reduced pressure and diluted with EtOAc (200 mL). Then the mixture was washed with sat. NaHCO₃ (10 mL×3). The organic layers were dried over anhydrous Na₂SO₄ and concentrated under reduced pressure to give a residue. The residue was purified by column chromatography (SiO₂, Petroleum ether/EtOAc=1:0 to 5:1). The crude product 5,6-dichloro-4-fluoro-1H-pyrrolo[2,3-b]pyridine (2.4 g, 9.95 mmol, 48.22% yield, 85% purity) as white solid was obtained.

### Synthesis of 5,6-dichloro-4-fluoro-1-(p-tolylsulfonyl)pyrrolo[2,3-b]pyridine

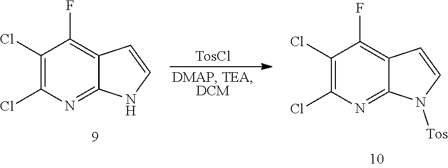

To a solution of 5,6-dichloro-4-fluoro-1H-pyrrolo[2,3-b]pyridine (580 mg, 2.83 mmol, 1 eq) and NaH (565.75 mg, 14.15 mmol, 60% purity, 5 eq) in THE (8 mL) was added TosCl (1.08 g, 5.66 mmol, 2 eq) under N₂. The mixture was stirred at 25° C. for 12 h. TLC and LCMS showed the desired MS was detected. The reaction mixture was quenched by addition saturated aqueous NH₄Cl (50 mL) at 0° C., and then extracted with EtOAc (50 mL×3). The combined organic layers were washed with brine (50 mL), dried over anhydrous Na₂SO₄, filtered and concentrated under reduced pressure to give a residue. The residue was purified by column chromatography (SiO₂, Petroleum ether/EtOAc=1:0 to 10:1). The product 1 5,6-dichloro-4-fluoro-1-(p-tolylsulfonyl)pyrrolo[2,3-b]pyridine (600 mg, 1.67 mmol, 59.04% yield) was obtained as white solid.

### Synthesis of 5-chloro-4-fluoro-6-methyl-1-(p-tolylsulfonyl)pyrrolo[2,3-b]pyridine

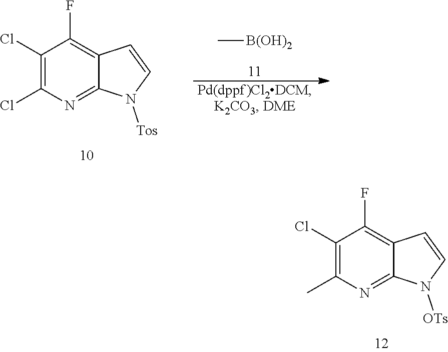

To a solution of methylboronic acid (1.09 g, 18.23 mmol, 5 eq) in DME (6 mL) and H2O (0.6 mL) was added 5,6-dichloro-4-fluoro-1-(p-tolylsulfonyl)pyrrolo[2,3-b]pyridine (1.31 g, 3.65 mmol, 1 eq) Pd(dppf)Cl₂.CH₂Cl₂ (297.83 mg, 364.70 umol, 0.1 eq) and Na₂CO₃ (1.16 g, 10.94 mmol, 3 eq). The mixture was stirred at 120° C. for 12 hr. TLC and LCMS showed the desired MS was detected and the reactant 10 was consumed. The mixture was concentrated under reduced pressure to give the residue. The residue was purified by column chromatography (SiO₂, Petroleum ether/EtOAc=1:0 to 10:1). The product 5-chloro-4-fluoro-6-methyl-1-(p-tolylsulfonyl)pyrrolo[2,3-b]pyridine (680 mg, 2.01 mmol, 55.04% yield, 100% purity) was obtained as white solid.

### Synthesis of 5-chloro-4-fluoro-6-methyl-1-(p-tolylsulfonyl)pyrrolo[2,3-b]pyridine-2-carboxylic acid

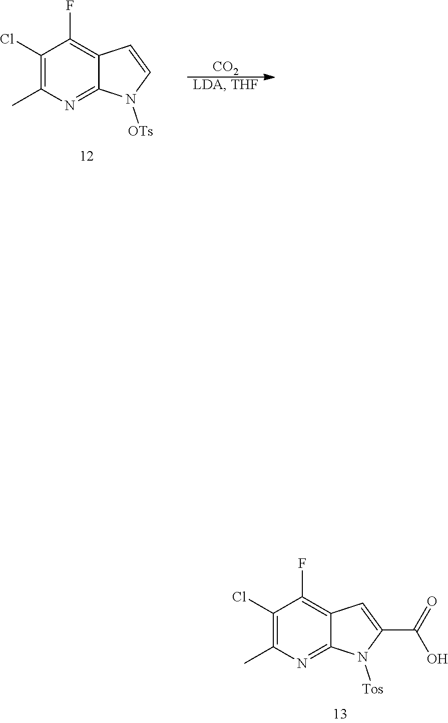

To a solution of CO2 (88.33 mg, 2.01 mmol, 1 eq) in THE (8 mL) was added LDA (2 M, 1.51 mL, 1.5 eq), the mixture was stirred at −78° C. for 1 h under N₂, then 5-chloro-4-fluoro-6-methyl-1-(p-tolylsulfonyl)pyrrolo[2,3-b]pyridine (680 mg, 2.01 mmol, 1 eq) was added and the mixture was stirred at the same temperature for 0.5 h. LCMS showed the desired MS was detected. The reaction was quenched at −78° C. with saturated aqueous NH₄Cl (30 mL) concentrated under reduced pressure to remove the THF. Then acidified with HCl (2 M) to pH=5. Then extracted with EtOAc (50 mL×3). The combined organic layers were washed with brine (50 mL), dried over anhydrous Na₂SO₄, filtered and concentrated under reduced pressure to give a residue. After concentration, the crude product was used directly for the next step without purification. The crude product 5-chloro-4-fluoro-6-methyl-1-(p-tolylsulfonyl)pyrrolo[2,3-b]pyridine-2-carboxylic acid (700 mg, 1.83 mmol, 91.11% yield) was obtained as brown solid. LCMS (ESI) m/z 382.9 [M+H]⁺

### Synthesis of 5-chloro-4-fluoro-6-methyl-1H-pyrrolo[2,3-b]pyridine-2-carboxylic acid

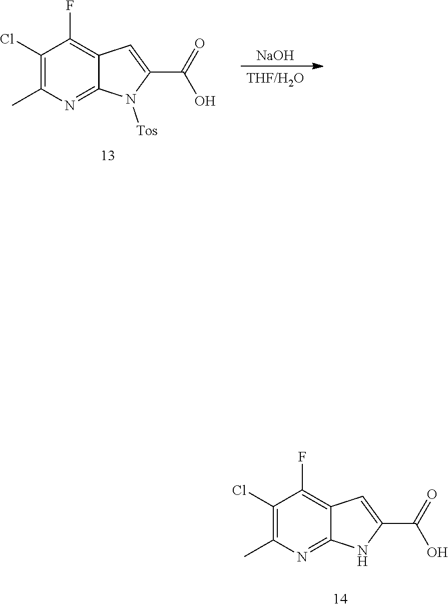

To a solution of 5-chloro-4-fluoro-6-methyl-1-(p-tolylsulfonyl)pyrrolo[2,3-b]pyridine-2-carboxylic acid (700 mg, 1.83 mmol, 1 eq) in THE (4 mL) was added NaOH (2 M, 4.57 mL, 5 eq) (in water), the mixture was stirred at 75° C. for 3 hr. LC-MS showed the starting material 13 was consumed completely. The mixture was concentrated under reduced pressure to give a residue, then diluted with water (10 mL), acidified with HCl (2 M) to pH=5. The mixture was filtered and the filter cake was washed with 10 mL×3 of Petroleum ether, dried under reduced pressure to give the crude product. The crude product was purified by washing with EtOAc (5 mL). The product 5-chloro-4-fluoro-6-methyl-1H-pyrrolo[2,3-b]pyridine-2-carboxylic acid (200 mg, 437.43 umol, 23.92% yield, 50% purity) was obtained as a brown solid. LCMS (ESI) m/z 228.9 [M+H]⁺

### Synthesis of 5-chloro-4-fluoro-6-methyl-N-[(1S,2S,3S,5R)-2,6,6-trimethylnorpinan-3-yl]-1H-pyrrolo[2,3-b]pyridine-2-carboxamide

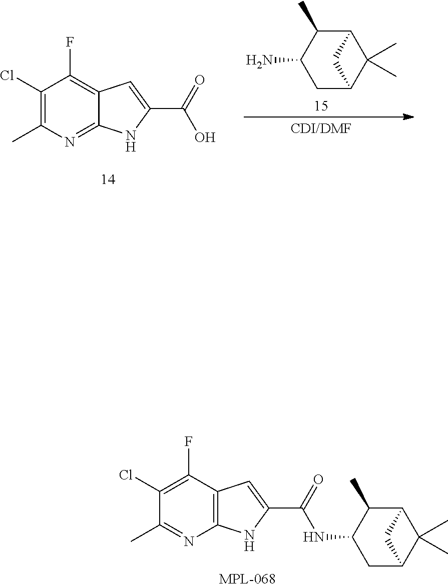

To a solution of 5-chloro-4-fluoro-6-methyl-1H-pyrrolo[2,3-b]pyridine-2-carboxylic acid (80 mg, 349.95 umol, 1 eq) in DMF (1 mL) was added CDI (85.12 mg, 524.92 umol, 1.5 eq), the mixture was stirred at 25° C. for 0.5 h, then (1S,2S,3S,5R)-2,6,6-trimethylnorpinan-3-amine (80.45 mg, 524.92 umol, 1.5 eq) was added, the mixture was stirred at 25° C. for 0.5 h. LCMS showed the reaction was consumed and the desired MS was detected. The residue was purified by Prep-HPLC (column: YMC-Actus Triart C18 150*30 mm*5 um; mobile phase: [water (0.225% FA)-ACN]; B %: 60%-82%, 11 min) without workup. The product 5-chloro-4-fluoro-6-methyl-N-[(1S,2S,3S,5R)-2,6,6-trimethylnorpinan-3-yl]-1H-pyrrolo[2,3-b]pyridine-2-carboxamide (4.7 mg, 12.19 umol, 3.48% yield, 94.392% purity) was obtained as white solid.

LCMS (ESI) m/z 364.2 [M+H]⁺; ¹H NMR (500 MHz, DMSO-d₆) δ=12.50 (br s, 1H), 8.44 (d, J=8.4 Hz, 1H), 7.26 (d, J=2.1 Hz, 1H), 4.35 (br t, J=8.5 Hz, 1H), 2.62 (s, 3H), 2.47-2.37 (m, 2H), 2.06 (quin, J=6.9 Hz, 1H), 1.94 (br s, 1H), 1.81 (br t, J=5.2 Hz, 1H), 1.69 (ddd, J=2.1, 6.4, 13.7 Hz, 1H), 1.23 (s, 3H), 1.18 (d, J=9.5 Hz, 1H), 1.07-1.04 (m, 6H).

### Example 16. MPL-108

### Synthesis of 4-(trifluoromethyl)-N-(1,7,7-trimethylnorbornan-2-yl)-1H-pyrrolo[2,3-b]pyridine-2-carboxamide

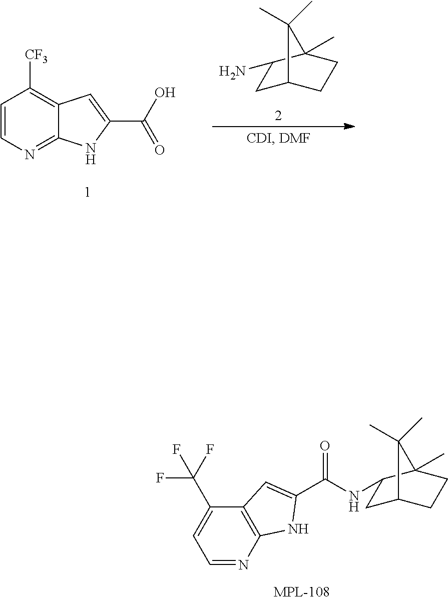

To a solution of 4-(trifluoromethyl)-1H-pyrrolo[2,3-b]pyridine-2-carboxylic acid (100 mg, 434.51 umol, 1 eq) in DMF (3.0 mL) was added CDI (105.68 mg, 651.77 umol, 1.5 eq) and stirred at 25° C. for 15 min. Then, 1,7,7-trimethylnorbornan-2-amine (113.21 mg, 738.67 umol, 1.7 eq) was added above solution and stirred at 25° C. for 12 h. LCMS showed the starting material was consumed completely and the desired MS was detected. The mixture was added water (10 mL) and extracted with EtOAc (15 mL×3) and the organic phase was washed with water (10 mL×3) and brine (10 mL×3) and dried over Na₂SO₄ and filtered and concentrated under reduced pressure to give a residue. The residue was purified by column chromatography (SiO₂, DCM/MeOH=1/0 to 200/1). Compound 4-(trifluoromethyl)-N-(1,7,7-trimethylnorbornan-2-yl)-1H-pyrrolo[2,3-b]pyridine-2-carboxamide (141.2 mg, 382.38 umol, 88.00% yield, 98.952% purity) was obtained as a white solid. LCMS (ESI) m/z: 366.2 [M+H]⁺

¹H NMR (500 MHz, DMSO-d₆) δ=12.77 (br s, 1H), 8.57 (d, J=4.9 Hz, 1H), 8.32 (br d, J=8.5 Hz, 1H), 7.55-7.43 (m, 2H), 4.43 (br s, 1H), 2.28-2.16 (m, 1H), 1.83-1.67 (m, 3H), 1.47-1.39 (m, 1H), 1.29 (br t, J=12.1 Hz, 1H), 1.18 (dd, J=4.9, 13.0 Hz, 1H), 1.02-0.95 (m, 3H), 0.88 (s, 3H), 0.80 (s, 3H).

### Example 17. MPL-119

### Synthesis of 4-chloro-N-(4,4-dimethylcyclohexyl)-6-methyl-1H-pyrrolo[2,3-b]pyridine-2-carboxamide

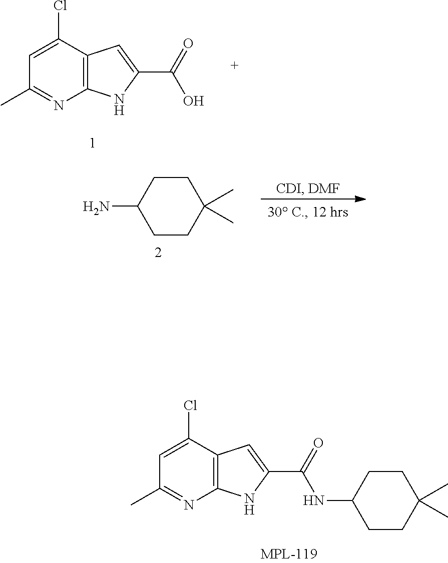

To a solution of 4-chloro-6-methyl-1H-pyrrolo[2,3-b]pyridine-2-carboxylic acid (75 mg, 356.10 umol, 1 eq) in DMF (2.5 mL) was added CDI (92.39 mg, 569.75 umol, 1.6 eq) and stirred at 30° C. for 1 hr. Then, 4,4-dimethylcyclohexanamine (58.90 mg, 462.93 umol, 1.3 eq) was added above solution and stirred at 30° C. for 11 hr. LCMS showed the starting material was consumed completely and the desired MS was detected. The mixture was added water (10 mL) and extracted with EtOAc (15 mL×3). The organic phase was washed with water (10 mL×3), brine (10 mL×3), dried over Na₂SO₄ and filtered and concentrated under reduced pressure to give a residue. The residue was purified by column chromatography (SiO₂, DCM:MeOH=1:0 to 300:1). Compound 4-chloro-N-(4,4-dimethylcyclohexyl)-6-methyl-1H-pyrrolo[2,3-b]pyridine-2-carboxamide (58.1 mg, 181.50 umol, 50.97% yield, 99.913% purity) was obtained as a white solid.

LCMS m/z: 320.1 [M+1]⁺; ¹H NMR (400 MHz, MeOD) δ=12.29 (s, 1H), 8.34 (br d, J=7.8 Hz, 1H), 7.23-7.15 (m, 2H), 3.79-3.66 (m, 1H), 2.54 (s, 3H), 1.67 (br dd, J=3.2, 12.8 Hz, 2H), 1.59-1.49 (m, 2H), 1.42 (br d, J=12.8 Hz, 2H), 1.28 (dt, J=3.2, 13.1 Hz, 2H), 0.96 (s, 3H), 0.93 (s, 3H).

### Example 18. MPL-126

### Synthesis of 5-chloro-N-[(1S,2S,3S,5R)-2,6,6-trimethylnorpinan-3-yl]-1H-pyrrolo[2,3-c]pyridine-2-carboxamide

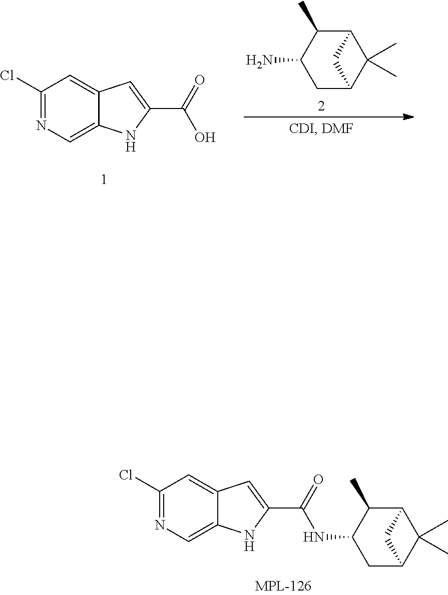

To a solution of 5-chloro-1H-pyrrolo[2,3-c]pyridine-2-carboxylic acid (80 mg, 406.94 umol, 1 eq) in DMF (2.0 mL) was added CDI (92.38 mg, 569.71 umol, 1.4 eq) and stirred at 30° C. for 1 h. Then, (1S,2S,3S,5R)-2,6,6-trimethylnorpinan-3-amine (106.03 mg, 691.79 umol, 1.7 eq) was added above solution and stirred at 30° C. for 2 h. LCMS showed the starting material was consumed completely and the desired MS was detected. The mixture was added water (10 mL) and extracted with EtOAc (15 mL×3). The organic phase was washed with water (10 mL×3) and brine (10 mL×3), dried over Na₂SO₄ and filtered and concentrated under reduced pressure to give a residue. The residue was purified by column chromatography (SiO₂, DCM:MeOH=1/0 to 200:1). Compound 5-chloro-N-[(1S,2S,3S,5R)-2,6,6-trimethylnorpinan-3-yl]-1H-pyrrolo[2,3-c]pyridine-2-carboxamide (62.4 mg, 187.50 umol, 46.08% yield, 99.713% purity) was obtained as a white solid. LCMS (ESI) m/z 332.2 [M+H]⁺; ¹H NMR (500 MHz, DMSO-d₆) δ=12.26 (s, 1H), 8.67 (br d, J=8.4 Hz, 1H), 8.58 (s, 1H), 7.78 (s, 1H), 7.25 (s, 1H), 4.40 (td, J=7.9, 16.4 Hz, 1H), 2.47-2.34 (m, 2H), 2.10 (quin, J=6.9 Hz, 1H), 1.96 (br s, 1H), 1.82 (br t, J=5.6 Hz, 1H), 1.72 (br dd, J=6.4, 12.2 Hz, 1H), 1.26-1.19 (m, 4H), 1.10-1.03 (m, 6H).

### Example 19. MPL-127

### Synthesis of 5-chloro-N-(4,4-dimethylcyclohexyl)-1H-pyrrolo[2,3-c]pyridine-2-carboxamide

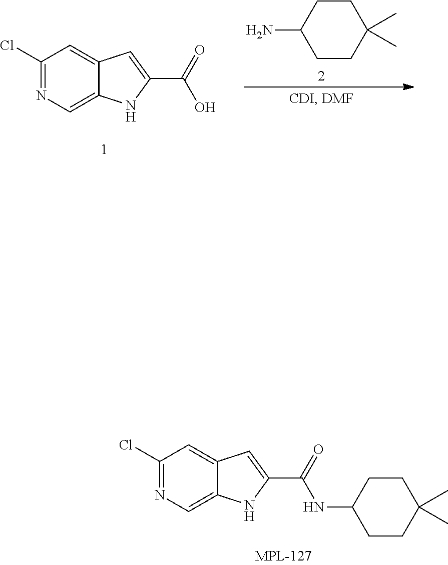

To a solution of 5-chloro-1H-pyrrolo[2,3-c]pyridine-2-carboxylic acid (80 mg, 406.94 umol, 1 eq) in DMF (2.0 mL) was added CDI (92.38 mg, 569.71 umol, 1.4 eq) and stirred at 30° C. for 1 h. Then, 4,4-dimethylcyclohexanamine (88.01 mg, 691.79 umol, 1.7 eq) was added above solution and stirred at 30° C. for 2 h. LCMS showed the starting material was consumed completely and the desired MS was detected. The mixture was added water (10 mL) and extracted with EtOAc (15 mL×3). The organic phase was washed with water (10 mL×3) and brine (10 mL×3), dried over Na₂SO₄ and filtered and concentrated under reduced pressure to give a residue. The residue was purified by column chromatography (SiO₂, DCM:MeOH=1/0 to 200:1). Compound 5-chloro-N-(4,4-dimethylcyclohexyl)-1H-pyrrolo[2,3-c]pyridine-2-carboxamide (57.3 mg, 186.63 umol, 45.86% yield, 99.604% purity) was obtained as a white solid. The purity and structure of product was confirmed by LCMS and ¹H NMR.

LCMS (ESI) m/z 306.1 [M+H]⁺; ¹H NMR (500 MHz, DMSO-d₆) δ=12.26 (s, 1H), 8.62-8.51 (m, 2H), 7.76 (s, 1H), 7.20 (s, 1H), 3.83-3.68 (m, 1H), 1.68 (br dd, J=3.1, 12.7 Hz, 2H), 1.60-1.50 (m, 2H), 1.42 (br d, J=13.0 Hz, 2H), 1.34-1.24 (m, 2H), 0.96 (s, 3H), 0.94 (s, 3H).

### Example 19a. MPL-136

### Synthesis of 5,7-dimethyl-N-[(1S,2S,3S,5R)-2,6,6-trimethylnorpinan-3-yl]-1H-pyrrolo[2,3-c]pyridine-2-carboxamide

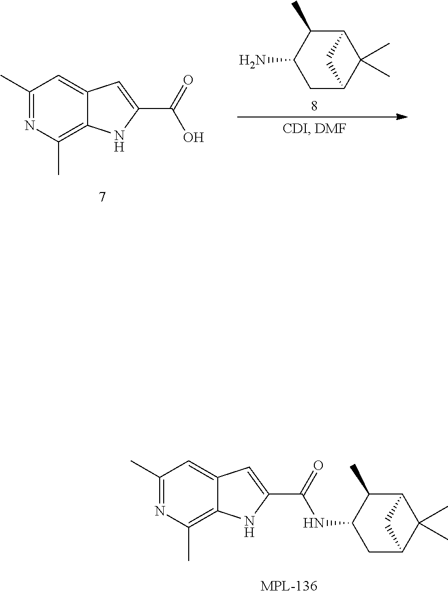

CDI (59.68 mg, 368.04 umol, 1.4 eq) was added to a solution of 5,7-dimethyl-1H-pyrrolo[2,3-c]pyridine-2-carboxylic acid (50 mg, 262.88 umol, 1 eq) in DMF (2.0 mL) and stirred at 30° C. for 0.5 h. Then, (1S,2S,3S,5R)-2,6,6-trimethylnorpinan-3-amine (68.49 mg, 446.90 umol, 1.7 eq) was added above solution and stirred at 30° C. for 12 h. LCMS showed the desired MS was detected. The mixture was added water (10 mL) and extracted with EtOAc (15 mL×3) and the organic phase was washed with water (10 mL×3) and brine (10 mL×3) and dried over Na₂SO₄ and filtered and concentrated under reduced pressure to give a residue. The residue was purified by prep-TLC (SiO₂, DCM:MeOH=10:1). Compound 5,7-dimethyl-N-[(1S,2S,3S,5R)-2,6,6-trimethylnorpinan-3-yl]-1H-pyrrolo[2,3-c]pyridine-2-carboxamide (13.6 mg, 41.79 umol, 15.90% yield, 100% purity) was obtained as a white solid.

LCMS (ESI) m/z: 326.2 [M+H]⁺; ¹H NMR (400 MHz, DMSO-d6) δ=11.84 (br s, 1H), 8.49 (br d, J=8.6 Hz, 1H), 7.25 (s, 1H), 7.07 (s, 1H), 4.45-4.29 (m, 1H), 2.66 (s, 3H), 2.43 (s, 3H), 2.42-2.28 (m, 2H), 2.12-2.02 (m, 1H), 1.93 (br s, 1H), 1.79 (br t, J=5.1 Hz, 1H), 1.74-1.65 (m, 1H), 1.22-1.18 (m, 4H), 1.07-1.02 (m, 6H).

### Example 20. MPL-137

### Synthesis of N-(4,4-dimethylcyclohexyl)-5,7-dimethyl-1H-pyrrolo[2,3-c]pyridine-2-carboxamide

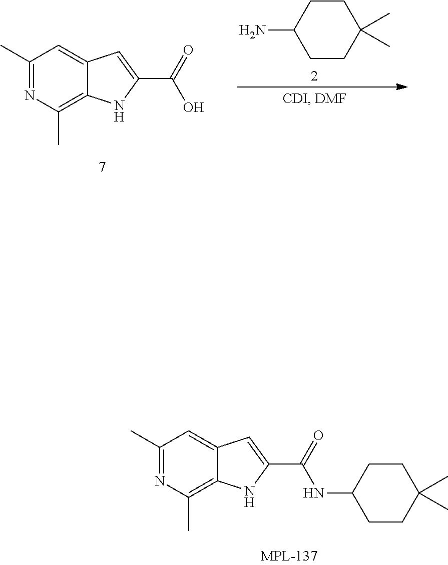

CDI (55.41 mg, 341.75 umol, 1.3 eq) was added to a solution of 5,7-dimethyl-1H-pyrrolo[2,3-c]pyridine-2-carboxylic acid (50 mg, 262.88 umol, 1 eq) in DMF (2.0 mL) and stirred at 30° C. for 0.5 h. Then, 4,4-dimethylcyclohexanamine (50.17 mg, 394.33 umol, 1.5 eq) was added above solution and stirred at 30° C. for 12 h. LCMS showed the starting material was consumed completely and the desired MS was detected. The mixture was added water (10 mL) and extracted with EtOAc (15 mL×3) and the organic phase was washed with water (10 mL×3) and brine (10 mL×3) and dried over Na₂SO₄ and filtered and concentrated under reduced pressure to give a residue. The residue was purified by prep-HPLC (column: YMC-Actus Triart C18 150*30 mm*5 um; mobile phase: [water (0.225% FA)-ACN]; B %: 25%-50%, 11 min). The obtained compound was not pure. The residue was purified by prep-TLC (SiO₂, DCM:MeOH=13:1). Compound N-(4,4-dimethylcyclohexyl)-5,7-dimethyl-1H-pyrrolo[2,3-c]pyridine-2-carboxamide (6.4 mg, 21.38 umol, 8.13% yield, 100% purity) was obtained as a white solid.

LCMS (ESI) m/z: 300.2 [M+H]⁺; ¹H NMR (400 MHz, DMSO-d6) δ=11.87 (br s, 1H), 8.39 (br d, J=8.1 Hz, 1H), 7.26 (s, 1H), 7.06 (s, 1H), 3.83-3.66 (m, 1H), 2.69 (s, 3H), 2.45 (s, 3H), 1.72-1.63 (m, 2H), 1.61-1.47 (m, 2H), 1.45-1.37 (m, 2H), 1.30 (br dd, J=3.5, 13.2 Hz, 2H), 0.94 (d, J=8.7 Hz, 6H).

### Example 21. MPL-140

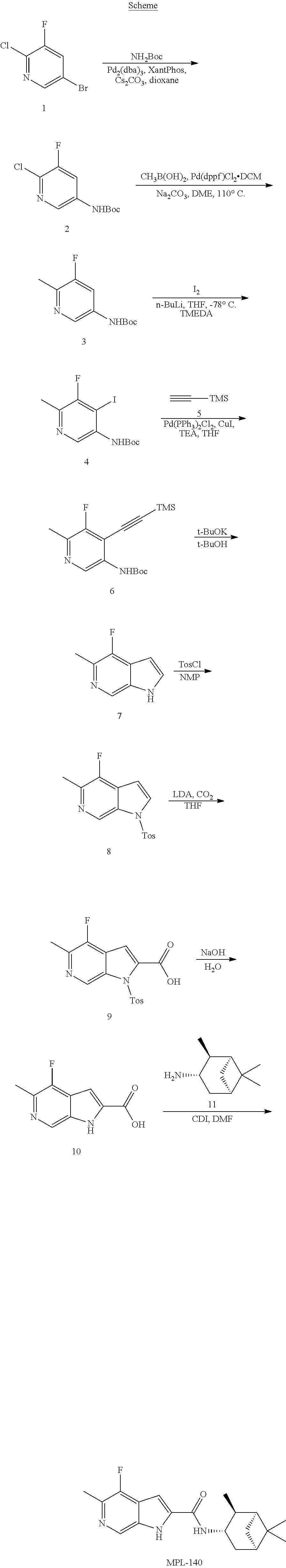

### Synthesis of tert-butyl N-(6-chloro-5-fluoro-3-pyridyl)carbamate

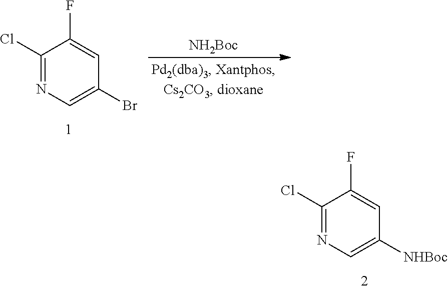 [SMILES: Fc1cc(Br)cnc1Cl ; CC(C)(C)OC(=O)Nc1cnc(Cl)c(F)c1 ; "NH2Boc"]

To a solution of 5-bromo-2-chloro-3-fluoro-pyridine (16 g, 76.03 mmol, 1 eq) and tert-butyl carbamate (9.35 g, 79.84 mmol, 1.05 eq) in dioxane (200 mL) was added Pd₂(dba)₃ (2.09 g, 2.28 mmol, 0.03 eq) Xantphos (4.40 g, 7.60 mmol, 0.1 eq) and Cs₂CO₃ (49.55 g, 152.07 mmol, 2 eq). The mixture was stirred at 85° C. for 24 hr under N₂. TLC and LC-MS showed the starting material was consumed completely and one main peak with desired MS was detected. The mixture was diluted with EtOAc (100 ml) and washed with H₂O (50 mL×3). The organic layers were dried over anhydrous Na₂SO₄ and concentrated under reduced pressure to give a residue. The residue was purified by column chromatography (SiO₂, Petroleum ether/EtOAc=1; 0 to 5:1). The product tert-butyl N-(6-chloro-5-fluoro-3-pyridyl)carbamate (16.7 g, 47.39 mmol, 62.33% yield, 70% purity) was obtained as yellow solid. LCMS (ESI) m/z 247.0 [M+H]⁺

### Synthesis of tert-butyl N-(5-fluoro-6-methyl-3-pyridyl)carbamate

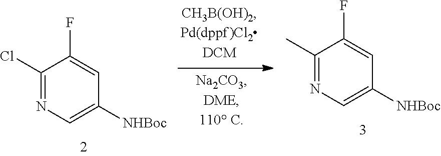 [SMILES: CC(C)(C)OC(=O)Nc1cnc(Cl)c(F)c1 ; Cc1ncc(NC(=O)OC(C)(C)C)cc1F]

To a solution of methylboronic acid (18.20 g, 304.06 mmol, 5 eq) in DME (200 mL) and H₂O (20 mL) was added tert-butyl N-(6-chloro-5-fluoro-3-pyridyl)carbamate (15 g, 60.81 mmol, 1 eq) Pd(dppf)Cl₂.CH₂Cl₂ (2.48 g, 3.04 mmol, 0.05 eq) and Na₂CO₃ (19.34 g, 182.43 mmol, 3 eq). The mixture was stirred at 120° C. for 36 hr. TLC and LCMS showed the desired MS was detected. The mixture was filtered and the filter was washed with brine (100 mL×2), dried over anhydrous Na₂SO₄, filtered and concentrated under reduced pressure to give a residue concentrated under reduced pressure to give the residue. The residue was purified by column chromatography (SiO2, Petroleum ether/EtOAc=1:0 to 5:1). The product tert-butyl N-(5-fluoro-6-methyl-3-pyridyl)carbamate (9.6 g, 38.19 mmol, 62.80% yield, 90% purity) was obtained as brown oil and purity comes from H NMR.

LCMS (ESI) m/z 227.2 [M+H]⁺

### Synthesis of tert-butyl N-(5-fluoro-4-iodo-6-methyl-3-pyridyl)carbamate

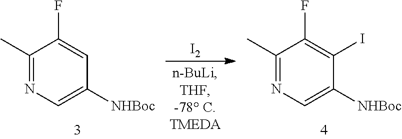 [SMILES: Cc1ncc(NC(=O)OC(C)(C)C)cc1F ; "I2" ; Cc1ncc(NC(=O)OC(C)(C)C)c(I)c1F]

To a solution of tert-butyl N-(5-fluoro-6-methyl-3-pyridyl)carbamate (3 g, 13.26 mmol, 1 eq) and TMEDA (4.62 g, 39.78 mmol, 6.00 mL, 3 eq) in THF (10 mL) was added n-BuLi (2.5 M, 26.52 mL, 5 eq) at −78° C. under N₂. The mixture was stirred for 0.5 h at the same temperature and the I₂ (10.10 g, 39.78 mmol, 8.01 mL, 3 eq) (in 20 ml THF) was dropwise added, the mixture was stirred for 11.5 h at the −78° C. under N₂. TLC and LC-MS showed the desired MS was detected. The reaction mixture was quenched by addition saturated aqueous NH₄Cl (50 mL) and saturated aqueous Na₂SO₃ (100 mL), and then extracted with EtOAc (100 mL×3). The combined organic layers were washed with brine (100 mL), dried over anhydrous Na₂SO₄, filtered and concentrated under reduced pressure to give a residue. The residue was purified by column chromatography (SiO₂, Petroleum ether/EtOAc=1:0 to 20:1). The product tert-butyl N-(5-fluoro-4-iodo-6-methyl-3-pyridyl)carbamate (3.6 g, 9.20 mmol, 69.39% yield, 90% purity) was obtained as white solid.

LCMS (ESI) m/z 352.9 [M+H]⁺

### Synthesis of tert-butyl N-[5-fluoro-6-methyl-4-(2-trimethylsilylethynyl)-3-pyridyl]carbamate

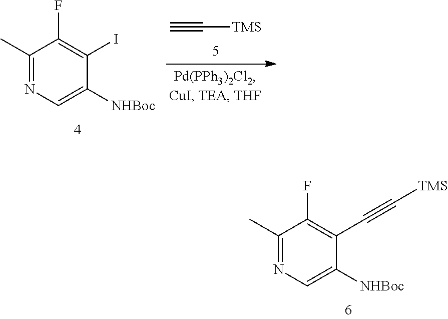

To a solution of tert-butyl N-(5-fluoro-4-iodo-6-methyl-3-pyridyl)carbamate (3.4 g, 9.66 mmol, 1 eq) in THF (30 mL) was added TEA (2.93 g, 28.97 mmol, 4.03 mL, 3 eq), CuI (367.77 mg, 1.93 mmol, 0.2 eq) and Pd(PPh3)₂Cl₂ (677.69 mg, 965.52 umol, 0.1 eq) under N₂. Then ethynyl(trimethyl)silane (2.84 g, 28.97 mmol, 4.01 mL, 3 eq) was added to the mixture, the mixture was stirred at 20° C. for 12 hr under N₂. TLC (Petroleum ether:EtOAc=5:1, Rf=0.5) indicated reactant was consumed completely and many new spots formed. The solvent was removed under reduced pressure to afford the crude product. The residue was purified by flash silica gel chromatography (ISCO®; 40 g SepaFlash® Silica Flash Column, Eluent of 0-5% EtOAc/Petroleum ether gradient at 40 mL/min). Compound tert-butyl N-[5-fluoro-6-methyl-4-(2-trimethylsilylethynyl)-3-pyridyl]carbamate (3.1 g, 6.73 mmol, 69.70% yield, 70% purity) was obtained as a brown solid.

### Synthesis of 4-fluoro-5-methyl-1H-pyrrolo[2,3-c]pyridine

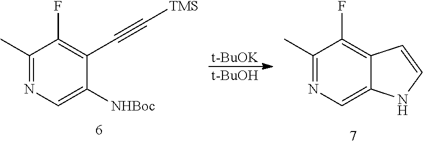 [SMILES: "t-BuOK" ; "t-BuOH" ; Cc1ncc(NC(=O)OC(C)(C)C)c(C#C[Si](C)(C)C)c1F ; Cc1ncc2[nH]ccc2c1F]

To a solution of tert-butyl N-[5-fluoro-6-methyl-4-(2-trimethylsilylethynyl)-3-pyridyl]carbamate (2.4 g, 7.44 mmol, 1 eq) in t-BuOH (50 mL) was added t-BuOK (2.51 g, 22.33 mmol, 3 eq). The mixture was stirred at 80° C. for 12 hr. TLC (Petroleum ether:EtOAc=2:1, Rf=0.2) indicated reactant was consumed completely, and one major new spot with larger polarity was detected. The mixture was used directly to the next step without work-up. Compound 4-fluoro-5-methyl-1H-pyrrolo[2,3-c]pyridine (1.12 g, crude) was in solution of t-BuOH.

### Synthesis of 4-fluoro-5-methyl-1-(p-tolylsulfonyl)pyrrolo[2,3-c]pyridine

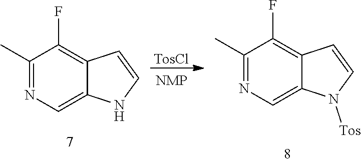

To a solution of 4-fluoro-5-methyl-1H-pyrrolo[2,3-c]pyridine (1.12 g, 7.46 mmol, 1 eq) in t-BuOH (50 mL) was added t-BuOK (2.51 g, 22.38 mmol, 3 eq) and 4-methylbenzenesulfonyl chloride (2.13 g, 11.19 mmol, 1.5 eq). The mixture was stirred at 20° C. for 12 hr. TLC indicated reactant was consumed completely and two new spots formed. LCMS showed one major peak with desired mass. The solvent was removed under reduced pressure, product was redissolved in EtOAc (20 mL), and organic layer was washed with water (20 mL) and Sat. NaCl (in water, 20 mL). The separated organic layer was dried over Na₂SO₄, filtered and concentrated to give the crude product which was purified by flash silica gel chromatography (ISCO®; 20 g SepaFlash® Silica Flash Column, Eluent of 0˜20% EtOAc/Petroleum ether gradient at 36 mL/min). Compound 4-fluoro-5-methyl-1-(p-tolylsulfonyl)pyrrolo[2,3-c]pyridine (1.98 g, 6.38 mmol, 85.48% yield, 98% purity) was obtained as a white solid. LCMS (ESI) m/z 305.1 [M+H]⁺

### Synthesis of 4-fluoro-5-methyl-1-(p-tolylsulfonyl)pyrrolo[2,3-c]pyridine-2-carboxylic acid

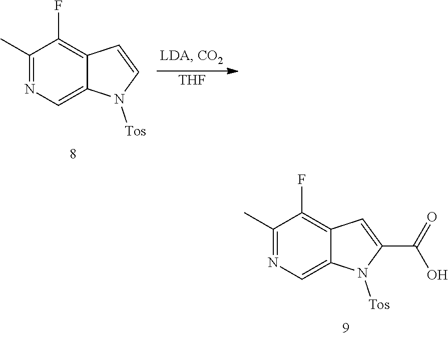

To a solution of 4-fluoro-5-methyl-1-(p-tolylsulfonyl)pyrrolo[2,3-c]pyridine (1.7 g, 5.59 mmol, 1 eq) in THE (20 mL) (dried by Na and distilled) was added LDA (2 M, 4.19 mL, 1.5 eq) dropwise at −78° C. under N₂. The mixture was stirred at −78° C. for 1.5 hr. Then N₂ balloon was exchanged with CO₂ balloon quickly, the mixture was allowed warm to 20° C. gradually and stirred under CO₂ for 12 hr. LC-MS showed reactant was consumed completely and two peaks which one of them with desired mass were detected. The reaction mixture was filtered under reduce pressure; filter cake was washed with EtOAc (10 mL×3). The product was used directly to the next step without further purification. Compound 4-fluoro-5-methyl-1-(p-tolylsulfonyl)pyrrolo[2,3-c]pyridine-2-carboxylic acid (2.1 g, crude) was obtained as a yellow solid. LCMS (ESI) m/z 349.0 [M+H]⁺

### Synthesis of 4-fluoro-5-methyl-1H-pyrrolo[2,3-c]pyridine-2-carboxylic acid

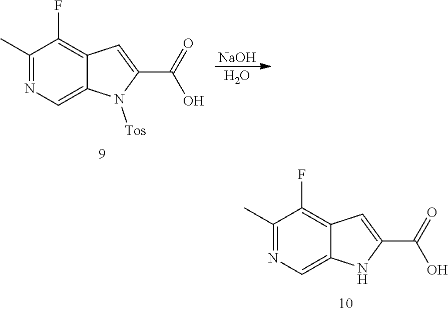

The 4-fluoro-5-methyl-1-(p-tolylsulfonyl)pyrrolo[2,3-c]pyridine-2-carboxylic acid (2.1 g, 6.03 mmol, 1 eq) was redissovled in NaOH (2 M, 20 mL, 6.63 eq). The mixture was stirred at 20° C. for 12 hr.

LC-MS showed reactant was consumed completely and one main peak with desired mass was detected. HCl (6 M, in water) was added into the reaction mixture to adjust pH=5. Filtered, the filter cake was washed with water (20 mL×2). Compound 4-fluoro-5-methyl-1H-pyrrolo[2,3-c]pyridine-2-carboxylic acid (545 mg, 2.75 mmol, 45.62% yield, 98% purity) was obtained as a white solid. LCMS (ESI) m/z 195.0 [M+H]⁺

### Synthesis of 4-fluoro-5-methyl-N-[(1S,2S,3S,5R)-2,6,6-trimethylnorpinan-3-yl]-1H-pyrrolo[2,3-c]pyridine-2-carboxamide

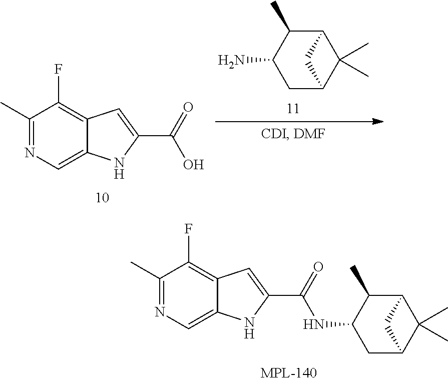

To a solution of 4-fluoro-5-methyl-1H-pyrrolo[2,3-c]pyridine-2-carboxylic acid (0.1 g, 515.03 umol, 1 eq) in DMF (3 mL) (dried by CaH₂) was added CDI (100.21 mg, 618.04 umol, 1.2 eq), the mixture was stirred at 20° C. for 0.5 hr. Then (1S,2S,3S,5R)-2,6,6-trimethylnorpinan-3-amine (94.72 mg, 618.04 umol, 1.2 eq) was added, the mixture was stirred at 20° C. for 1 hr. LC-MS showed reactant was consumed completely and one main peak with desired mass was detected. The reaction mixture was dropped into water (20 mL). The product was isolated as white solid. Filtered, the filter cake was washed with water (5 mL×v2) to give the crude product. The residue was purified by flash silica gel chromatography (ISCO®; 12 g SepaFlash® Silica Flash Column, Eluent of 0-50% EtOAc/Petroleum ether gradient at 40 mL/min). All fractions found to contain product by TLC (Petroleum ether:EtOAc=3:1, Rf=0.4) were combined and evaporated. Compound 4-fluoro-5-methyl-N-[(1S,2S,3S,5R)-2,6,6-trimethylnorpinan-3-yl]-1H-pyrrolo[2,3-c]pyridine-2-carboxamide (15 mg, 45.54 umol, 8.84% yield, 100% purity) was obtained as a white solid.

LCMS (ESI) m/z 330.2 [M+H]⁺; ¹H NMR (500 MHz, DMSO-d₆) δ=12.16 (br s, 1H) 8.50 (d, J=8.54 Hz, 1H) 8.43 (d, J=2.14 Hz, 1H) 7.21 (s, 1H) 4.25-4.34 (m, 1H) 2.38 (d, J=3.20 Hz, 3H) 2.32-2.36 (m, 1H) 2.25-2.31 (m, 1H) 1.94-2.04 (m, 1H) 1.81-1.89 (m, 1H) 1.72 (t, J=5.26 Hz, 1H) 1.62 (ddd, J=13.69, 6.45, 2.14 Hz, 1H) 1.14 (s, 3H) 1.11 (d, J=9.61 Hz, 1H) 0.95-1.00 (m, 6H).

### Example 22. MPL-160

### Synthesis of N-(4,4-dimethylcyclohex-2-en-1-yl)-4-fluoro-1H-pyrrolo[2,3-b]pyridine-2-carboxamide

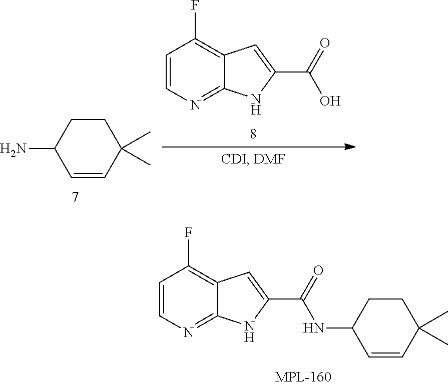

To a solution of 4-fluoro-1H-pyrrolo[2,3-b]pyridine-2-carboxylic acid (100 mg, 555.14 umol, 1 eq) in DMF (1 mL) was added 4,4-dimethylcyclohex-2-en-1-amine (116.67 mg, 721.68 umol, 1.3 eq, HCl), 1-methylimidazole (182.31 mg, 2.22 mmol, 177.00 uL, 4 eq) and [chloro(dimethylamino)methylene]-dimethyl-ammonium; hexafluorophosphate (202.49 mg, 721.68 umol, 1.3 eq). The mixture was stirred at 30° C. for 2 hr. LCMS showed there were trace starting material and main desired compound. The reaction was added dropwise to H₂O (20 mL). There was much precipitation which was collected by filter. The cake was diluted in EtOAc (20 mL), dried with anhydrous MgSO₄, filtered. The filtrate was concentrated in vacuo. The residue was purified by prep-HPLC (column: YMC-Actus Triart C18 150*30 mm*5 um; mobile phase: [water (0.225% FA)-ACN]; B %: 45%-70%, 11 min). Compound N-(4,4-dimethylcyclohex-2-en-1-yl)-4-fluoro-1H-pyrrolo[2,3-b]pyridine-2-carboxamide (20 mg, 69.61 umol, 12.54% yield, 100% purity) was obtained as a white solid which was confirmed by LCMS and ¹H NMR.

LCMS (ESI) m/z 288.1 [M+H]⁺; ¹H NMR (400 MHz, DMSO-d6) δ=12.44 (br s, 1H), 8.45 (br d, J=7.8 Hz, 1H), 8.30 (dd, J=5.4, 8.3 Hz, 1H), 7.28 (s, 1H), 6.99 (dd, J=5.4, 10.3 Hz, 1H), 5.58-5.52 (m, 1H), 5.48-5.42 (m, 1H), 4.49-4.40 (m, 1H), 1.84 (br d, J=5.4 Hz, 1H), 1.70-1.54 (m, 2H), 1.49-1.40 (m, 1H), 1.02 (s, 3H), 0.97 (s, 3H).

### Example 23. MPL-166

### Synthesis of N-(3-bicyclo[3.2.1]octanyl)-4-chloro-1H-pyrrolo[2,3-c]pyridine-2-carboxamide

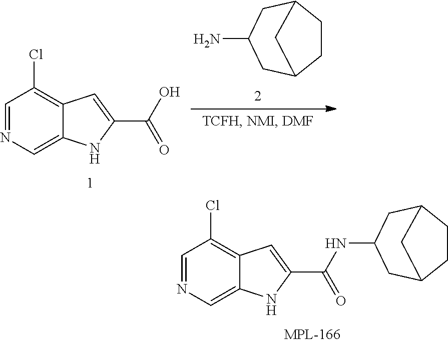 [SMILES: O=C(O)c1cc2c(Cl)cncc2[nH]1 ; NC1CC2CCC(C1)C2 ; O=C(NC1CC2CCC(C2)C1)c1cc2c(Cl)cncc2[nH]1]

To a solution of 4-chloro-1H-pyrrolo[2,3-c]pyridine-2-carboxylic acid (50 mg, 254.34 umol, 1 eq) in DMF (1 mL) was added bicyclo[3.2.1]octan-3-amine (49.34 mg, 305.20 umol, 1.2 eq, HCl), 1-methylimidazole (83.53 mg, 1.02 mmol, 81.09 uL, 4 eq) and [chloro(dimethylamino)methylene]-dimethyl-ammonium; hexafluorophosphate (92.77 mg, 330.64 umol, 1.3 eq). The mixture was stirred at 30° C. for 12 hr. LCMS showed there were no starting material and main desired compound. The reaction was added dropwise to H₂O (20 mL). There was much precipitation which was collected by filter. The cake was diluted in EtOAc (20 mL), dried with anhydrous MgSO₄, filtered. The filtrate was concentrated in vacuo. The residue was purified by prep-HPLC (column: YMC-Actus Triart C18 150*30 mm*5 um; mobile phase: [water (0.225% FA)-ACN]; B %: 30%-58%, 11 min). Compound N-(3-bicyclo[3.2.1]octanyl)-4-chloro-1H-pyrrolo[2,3-c]pyridine-2-carboxamide (20 mg, 65.84 umol, 25.89% yield, 100% purity) was obtained as a white solid.

LCMS (ESI) m/z 304.1 [M+H]⁺; ¹H NMR (400 MHz, DMSO-d₆) δ=12.48 (br s, 1H), 8.73 (s, 1H), 8.53 (br d, J=6.8 Hz, 1H), 8.19 (s, 1H), 7.32 (s, 1H), 4.20-4.07 (m, 1H), 2.24 (br s, 2H), 1.73 (br d, J=12.2 Hz, 2H), 1.69-1.58 (m, 2H), 1.52 (br d, J=7.6 Hz, 2H), 1.46-1.32 (m, 4H).

### Example 24. MPL-200

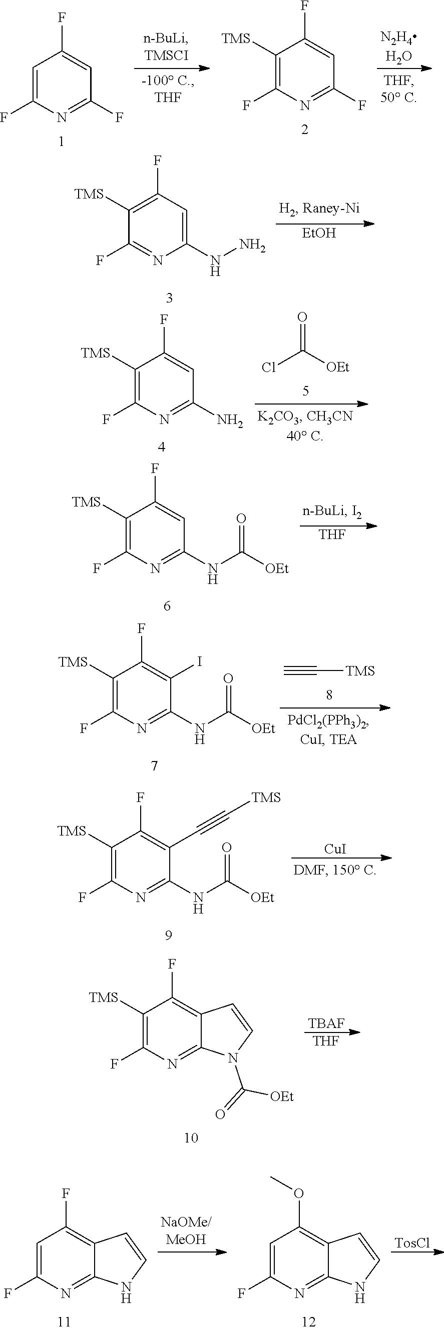

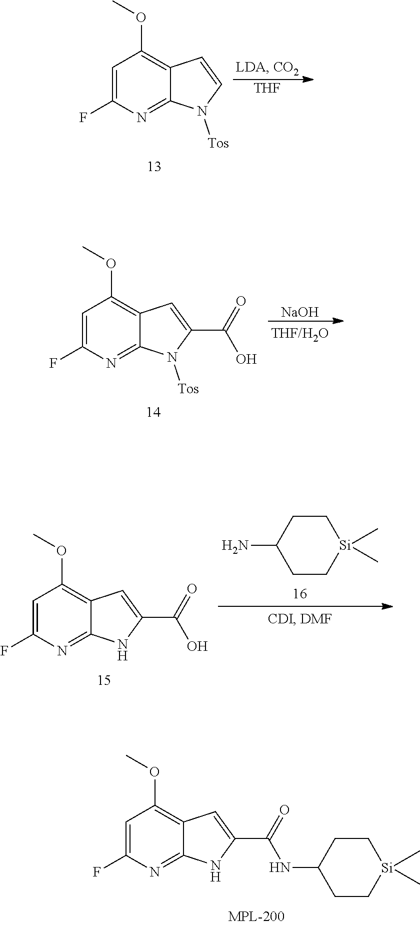

### Synthesis of trimethyl-(2,4,6-trifluoro-3-pyridyl)silane

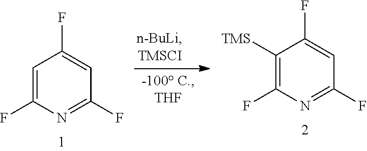 [SMILES: Fc1cc(F)nc(F)c1 ; C[Si](C)(C)c1c(F)cc(F)nc1F]

At −100° C. 2,4,6-trifluoropyridine (5 g, 37.57 mmol, 1 eq) in THF (78 mL) was added dropwise n-BuLi (2.5 M, 15.78 mL, 1.05 eq) in hexane. After 45 min at −100° C., chloro(trimethyl)silane (4.08 g, 37.57 mmol, 4.77 mL, 1 eq) was added in one portion, after 45 min at −75° C. TLC showed the starting material was consumed. The reaction solution was quenched by aq. sat. NH₄Cl (40 mL), extracted with petroleum ether (50 mL×2). The organic layers were dried over Na₂SO₄ and filtered and concentrated under reduced pressure to give a liquid. The liquid was purified by column chromatography (SiO₂, petroleum ether). Compound trimethyl-(2,4,6-trifluoro-3-pyridyl)silane (6.0 g, 27.77 mmol, 73.91% yield, 95% purity) was obtained as a colorless liquid.

### Synthesis of (4,6-difluoro-5-trimethylsilyl-2-pyridyl)hydrazine

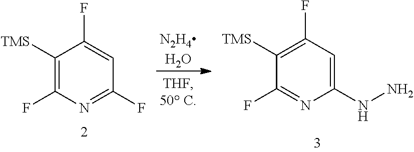 [SMILES: C[Si](C)(C)c1c(F)cc(F)nc1F ; "N2H4·H2O" ; C[Si](C)(C)c1c(F)cc(NN)nc1F]

To a solution of trimethyl-(2,4,6-trifluoro-3-pyridyl)silane (9 g, 43.85 mmol, 1 eq) in THF (80 mL) was added NH₂NH₂.H₂O (5.16 g, 87.70 mmol, 5.01 mL, 2 eq). The mixture was stirred at 50° C. for 2 hr. TLC showed the desired product was detected. The mixture was concentrated in reduced pressure. The residue was diluted with H₂O (50 mL). The aqueous phase was extracted with EtOAc (30 mL×3). The combined organic phase was washed with brine (10 mL×2), dried with anhydrous Na₂SO₄, filtered. The filtrate was concentrated in vacuo. The crude product was purified by silica column chromatography (SiO₂, petroleum ether/EtOAc=50/1 to 5/1). Compound (4,6-difluoro-5-trimethylsilyl-2-pyridyl)hydrazine (4.5 g, 18.64 mmol, 42.51% yield, 90% purity) was obtained as a yellow solid.

### Synthesis of 4,6-difluoro-5-trimethylsilyl-pyridin-2-amine

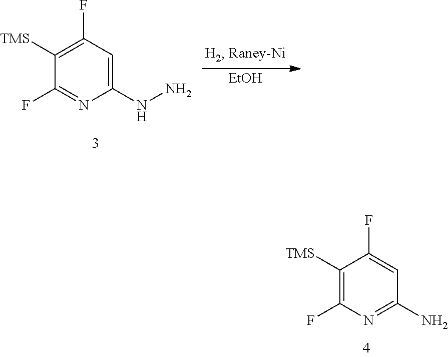 [SMILES: "EtOH" ; C[Si](C)(C)c1c(F)cc(NN)nc1F ; C[Si](C)(C)c1c(F)cc(N)nc1F]

To a solution of (4,6-difluoro-5-trimethylsilyl-2-pyridyl)hydrazine (5 g, 23.01 mmol, 1 eq) in EtOH (65 mL) was added Raney-Ni (394.29 mg, 2.30 mmol, 50% purity, 0.1 eq) under N₂. The suspension was degassed under vacuum and purged with H₂ several times. The mixture was stirred under H₂ (20 psi) at 30° C. for 48 hours. TLC showed the starting material was consumed. The mixture was filtered and the filter cake was washed with EtOAc (50 mL). The filtrate was concentrated under reduced pressure to give 4,6-difluoro-5-trimethylsilyl-pyridin-2-amine (4.58 g, 18.11 mmol, 78.72% yield, 80% purity) as a yellow solid.

### Synthesis of ethyl N-(4,6-difluoro-5-trimethylsilyl-2-pyridyl)carbamate

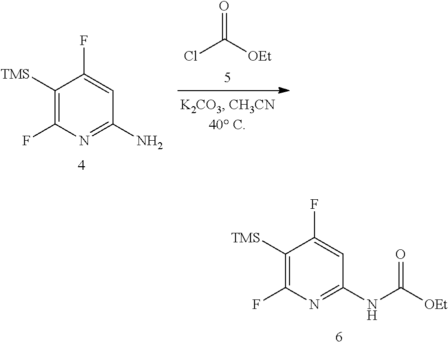 [SMILES: C[Si](C)(C)c1c(F)cc(N)nc1F ; CCOC(=O)Cl ; CCOC(=O)Nc1cc(F)c([Si](C)(C)C)c(F)n1]

To a solution of 4,6-difluoro-5-trimethylsilyl-pyridin-2-amine (4.58 g, 22.64 mmol, 1 eq) and Py (7.16 g, 90.57 mmol, 7.31 mL, 4 eq) in DCM (45 mL) was added ethyl carbonochloridate (9.83 g, 90.57 mmol, 8.62 mL, 4 eq) dropwise at 0° C. under N₂. The mixture was stirred at 20° C. for 15 min. TLC showed the starting material was consumed, and one new spot was formed. The mixture was quenched with sat. NaHCO₃ (20 mL), extracted with EtOAc (20 mL×2). The organic layers were washed with 0.5 M aq. HCl (20 mL×2) and dried over Na₂SO₄ and filtered and concentrated under reduced pressure to give a liquid. The liquid was purified by column chromatography (SiO₂, petroleum ether/EtOAc=100/1 to 10/1). Compound ethyl N-(4,6-difluoro-5-trimethylsilyl-2-pyridyl)carbamate (5.4 g, 17.72 mmol, 78.24% yield, 90% purity) was obtained as a yellow liquid.

### Synthesis of ethyl N-(4,6-difluoro-3-iodo-5-trimethylsilyl-2-pyridyl)carbamate

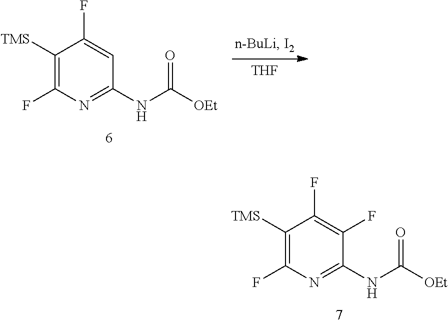 [SMILES: CCOC(=O)Nc1cc(F)c([Si](C)(C)C)c(F)n1 ; CCOC(=O)Nc1nc(F)c([Si](C)(C)C)c(F)c1F ; "THF"]

To a solution of ethyl N-(4,6-difluoro-5-trimethylsilyl-2-pyridyl)carbamate (4.4 g, 16.04 mmol, 1 eq) and TMEDA (3.73 g, 32.08 mmol, 4.84 mL, 2 eq) in THE (30 mL) was added n-BuLi (2.5 M, 12.83 mL, 2 eq) dropwise at −78° C. for 0.5 hr under N₂. Then I₂ (8.14 g, 32.08 mmol, 6.46 mL, 2 eq) in THE (14 mL) was added into above solution at −78° C. for 1 hr. TLC showed the starting material was consumed. The reaction was quenched by sat. NH₄Cl (50 mL), extracted with EtOAc (50 mL×3). The organic layers were dried over Na₂SO₄ and filtered and concentrated under reduced pressure to give a residue. The residue was purified by column chromatography (SiO₂, petroleum ether/EtOAc=100/1 to 10/1). Compound ethyl N-(4,6-difluoro-3-iodo-5-trimethylsilyl-2-pyridyl)carbamate (4.5 g, 10.12 mmol, 63.09% yield, 90% purity) was obtained as a yellow liquid.

### Synthesis of N-[4,6-difluoro-5-trimethylsilyl-3-(2-trimethylsilylethynyl)-2-pyridyl]carbamate

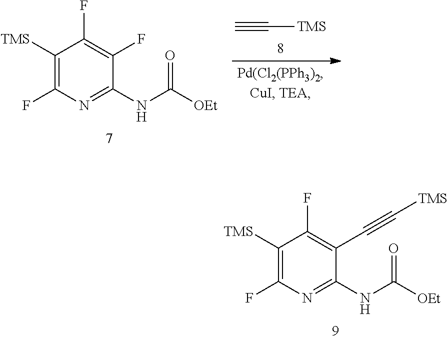

ethyl N-(4,6-difluoro-3-iodo-5-trimethylsilyl-2-pyridyl)carbamate (4.5 g, 11.24 mmol, 1 eq), ethynyl-trimethyl-silane (11.04 g, 112.43 mmol, 15.58 mL, 10 eq) and Pd(PPh3)₂Cl₂ (789.17 mg, 1.12 mmol, 0.1 eq), CuI (642.39 mg, 3.37 mmol, 0.3 eq) in TEA (45 mL) was de-gassed and then heated to 80° C. for 12 hours under N₂. LCMS showed the desired product was detected. The mixture was diluted with EtOAc (50 mL) and washed with water (50 mL) and aq. 1 M HCl (50 mL×2). The organic layer was dried over Na₂SO₄ and filtered and concentrated under reduced pressure to give a residue. The residue was purified by column chromatography (SiO₂, petroleum ether/EtOAc=100/1 to 10/1). Compound ethyl N-[4,6-difluoro-5-trimethylsilyl-3-(2-trimethylsilylethynyl)-2-pyridyl]carbamate (4 g, 9.18 mmol, 81.61% yield, 85% purity) was obtained as a yellow solid. LCMS (ESI), m/z 371.4 [M+H]⁺

### Synthesis of ethyl 4,6-difluoro-5-trimethylsilyl-pyrrolo[2,3-b]pyridine-1-carboxylate

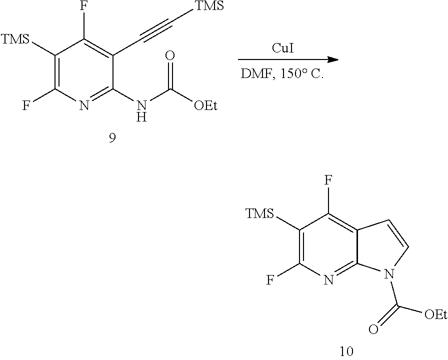 [SMILES: CCOC(=O)Nc1nc(F)c([Si](C)(C)C)c(F)c1C#C[Si](C)(C)C ; "CuI" ; CCOC(=O)n1ccc2c(F)c([Si](C)(C)C)c(F)nc21]

The mixture of ethyl N-[4,6-difluoro-5-trimethylsilyl-3-(2-trimethylsilylethynyl)-2-pyridyl]carbamate (4 g, 9.18 mmol, 1 eq) and CuI (3.50 g, 18.35 mmol, 2 eq) in DMF (40 mL) was stirred at 150° C. for 2 hr. LCMS showed the desired product was detected. The mixture was diluted with EtOAc (200 mL) and washed with 3% aq. LiCl (40 mL×2), following by brine (40 mL). The organic phase was dried over Na₂SO₄ and filtered and concentrated under reduced pressure to give a residue. The residue was purified by column chromatography (SiO₂, petroleum ether/EtOAc=100/1 to 10/1). Compound ethyl 4,6-difluoro-5-trimethylsilyl-pyrrolo[2,3-b]pyridine-1-carboxylate (1.1 g, 3.50 mmol, 38.14% yield, 95% purity) was obtained as a yellow solid. LCMS (ESI), m/z 371.4 [M+H]⁺

### Synthesis of 4,6-difluoro-1H-pyrrolo[2,3-b]pyridine

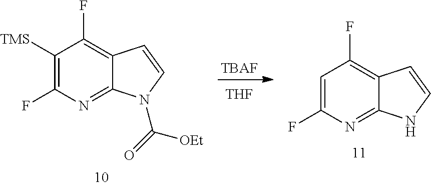

Ethyl 4,6-difluoro-5-trimethylsilyl-pyrrolo[2,3-b]pyridine-1-carboxylate (1.1 g, 3.69 mmol, 1 eq) was dissolved to TBAF (1 M, 11.00 mL, 2.98 eq) (In THF) was stirred at 25° C. for 12 hr. TLC showed the starting material was consumed. The mixture was quenched by water (20 mL) and extracted with EtOAc (30 mL×2). The organic layers were washed with brine (30 mL) dried over Na₂SO₄ and filtered and concentrated under reduced pressure to give a residue. The residue was purified by column chromatography (SiO₂, petroleum ether/EtOAc=100/1 to 10/1). Compound 4,6-difluoro-1H-pyrrolo[2,3-b]pyridine (560 mg, crude) was obtained as a yellow solid.

### Synthesis of 6-fluoro-4-methoxy-1H-pyrrolo[2,3-b]pyridine

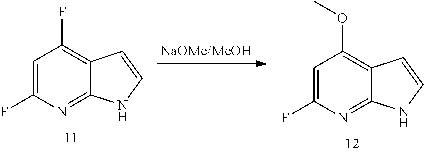

A mixture of 4,6-difluoro-1H-pyrrolo[2,3-b]pyridine (560 mg, 3.63 mmol, 1 eq) and NaOMe (392.60 mg, 7.27 mmol, 2 eq) in MeOH (5.6 mL) was stirred at 50° C. for 12 hr. LCMS showed the starting material was consumed and the desired product was detected. The mixture was diluted with EtOAc (10 mL) and washed with water (5 mL), follow by brine (5 mL). The organic layer was dried over Na₂SO₄ and filtered and concentrated under reduced pressure to give a residue. The residue was purified by prep-TLC (SiO₂, petroleum ether/EtOAc=3/1). Compound 6-fluoro-4-methoxy-1H-pyrrolo[2,3-b]pyridine (400 mg, 2.41 mmol, 66.25% yield, 100% purity) was obtained as a white solid. LCMS (ESI), m/z 166.9[M+H]⁺

### Synthesis of 6-fluoro-4-methoxy-1-(p-tolylsulfonyl)pyrrolo[2,3-b]pyridine

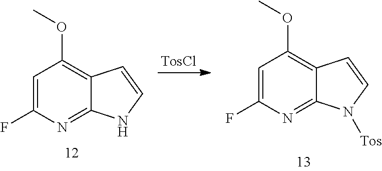

To a solution of 6-fluoro-4-methoxy-1H-pyrrolo[2,3-b]pyridine (444 mg, 2.67 mmol, 1 eq), TEA (811.22 mg, 8.02 mmol, 1.12 mL, 3 eq) and DMAP (65.29 mg, 534.45 umol, 0.2 eq) in THE (10 mL) was added TosCl (1.02 g, 5.34 mmol, 2 eq). The mixture was stirred at 30° C. for 12 hr. LCMS showed 75% desired product was detected and 25% starting material was remained. The mixture was diluted with EtOAc (40 mL) and washed with water (20 mL×2), follow by brine (20 mL). The organic layer was dried over Na₂SO₄ and filtered and concentrated under reduced pressure to give a residue. This residue was purified by column chromatography (SiO₂, petroleum ether/EtOAc=50/1 to 5/1). Compound 6-fluoro-4-methoxy-1-(p-tolylsulfonyl)pyrrolo[2,3-b]pyridine (686 mg, 1.61 mmol, 60.10% yield, 75% purity) as a yellow solid. LCMS (ESI), m/z 321.1 [M+H]⁺

### Synthesis of 6-fluoro-4-methoxy-1-(p-tolylsulfonyl)pyrrolo[2,3-b]pyridine-2-carboxylic acid

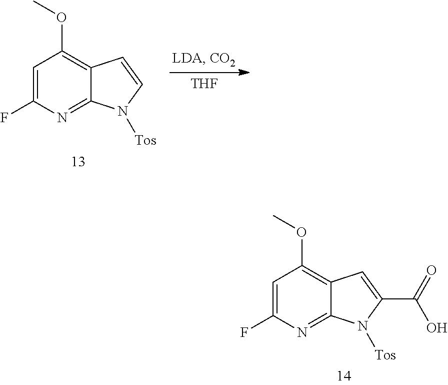

To a solution of 6-fluoro-4-methoxy-1-(p-tolylsulfonyl)pyrrolo[2,3-b]pyridine (450 mg, 1.40 mmol, 1 eq) in THF (6 mL) was added dropwise LDA (2 M, 1.40 mL, 2 eq) at −78° C. under N₂. The mixture was stirred at −78° C. for 1 hr under N2. Then the mixture was stirred at −78° C. for 0.5 hr under CO₂ (15 psi) atmosphere. LCMS showed there was no starting material and main desired compound was detected. The reaction was not worked up and the reaction solution was used into next step. LCMS (ESI), m/z 365.0 [M+H]⁺

### Synthesis of 6-fluoro-4-methoxy-1H-pyrrolo[2,3-b]pyridine-2-carboxylic acid

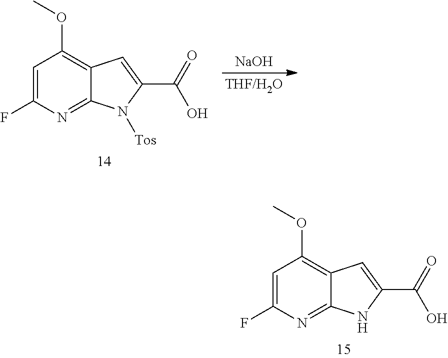

Aqueous solution NaOH (2 M, 6 mL, 8.56 eq) was added into 6-fluoro-4-methoxy-1-(p-tolylsulfonyl)pyrrolo[2,3-b]pyridine-2-carboxylic acid (511 mg, 1.40 mmol, 1 eq) in above step solution (6 m THF) under N₂ and stirred at 70° C. for 1 hr. LCMS showed the desired product was detected. The reaction solution was concentrated under reduced pressure to remove THF, and the aqueous solution was extracted with EtOAc (5 mL×2). The aqueous solution was neutralized with aq. 2 M HCl to pH=4. Then the precipitate was formed, filtered and the filter cake was collected. Compound 6-fluoro-4-methoxy-1H-pyrrolo[2,3-b]pyridine-2-carboxylic acid (120 mg, 342.59 umol, 24.43% yield, 60% purity) was obtained as a white solid. LCMS (ESI), m/z 211.0 [M+H]⁺

### Synthesis of N-(1,1-dimethylsilinan-4-yl)-6-fluoro-4-methoxy-1H-pyrrolo[2,3-b]pyridine-2-carboxamide

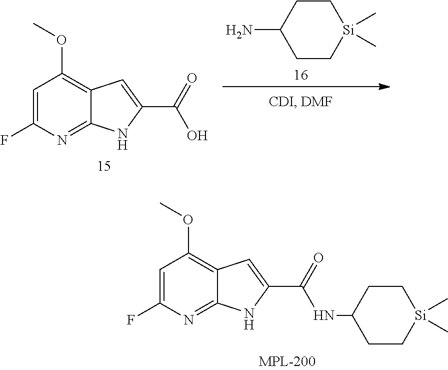

To a solution of 6-fluoro-4-methoxy-1H-pyrrolo[2,3-b]pyridine-2-carboxylic acid (120 mg, 570.99 umol, 1 eq) in DMF (2 mL) was added CDI (97.21 mg, 599.54 umol, 1.05 eq) under N₂, the mixture was stirred at 30° C. for 1 hr. 1,1-dimethylsilinan-4-amine (97.80 mg, 682.49 umol, 1.2 eq) was added to above solution and stirred at 30° C. for 1 hr. LCMS showed the desired product was detected. The mixture was purified by prep-HPLC (column: YMC-Actus Triart C18 150*30 mm*5 um; mobile phase: [water (0.225% FA)-ACN]; B %: 55%-85%, 10 min). Compound N-(1,1-dimethylsilinan-4-yl)-6-fluoro-4-methoxy-1H-pyrrolo[2,3-b]pyridine-2-carboxamide (44 mg, 130.98 umol, 23.03% yield, 99.86% purity) was obtained as a white solid.

LCMS (ESI), m/z 336.1 [M+H]⁺; 1H NMR (500 MHz, DMSO-d6) δ=12.06 (s, 1H), 8.04 (d, J=8.1 Hz, 1H), 7.10 (s, 1H), 6.38 (s, 1H), 3.97-3.81 (m, 3H), 3.70-3.48 (m, 1H), 1.89 (br d, J=9.3 Hz, 2H), 1.67-1.37 (m, 2H), 0.69 (br d, J=14.5 Hz, 2H), 0.51 (dt, J=4.7, 14.1 Hz, 2H), 0.02-−0.09 (m, 6H).

### Example 25. MPL-209

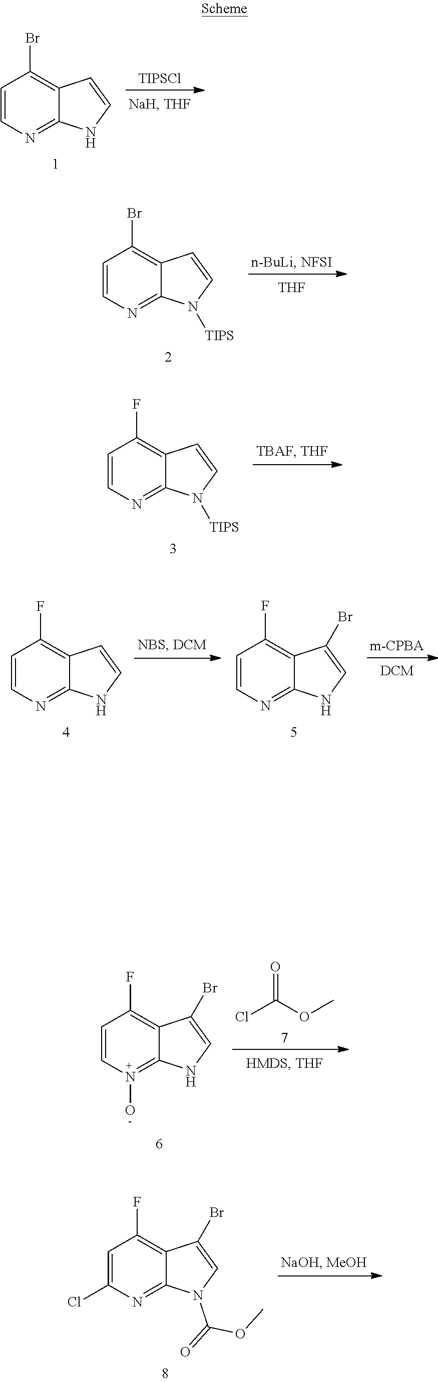

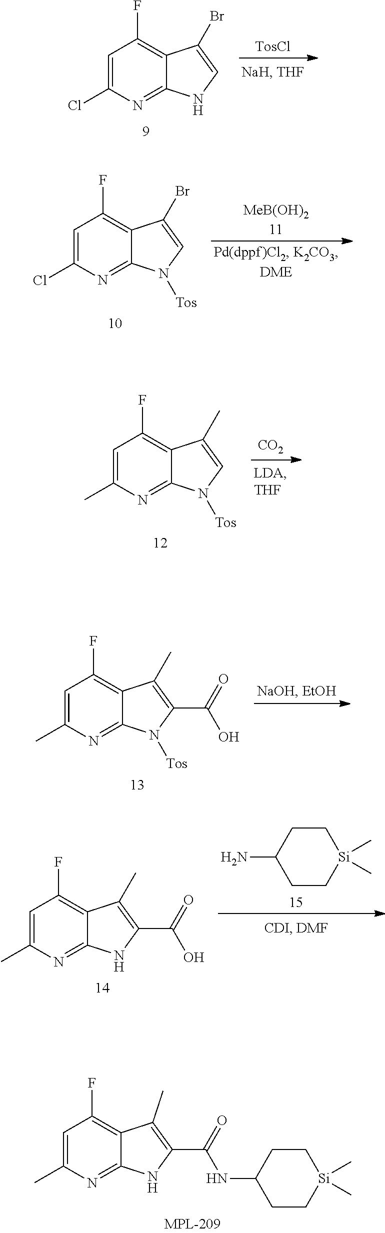

### (4-bromopyrrolo[2,3-b]pyridin-1-yl)-triisopropyl-silane

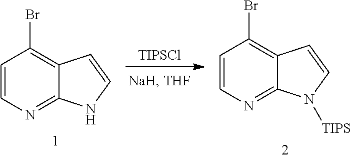

To a solution of NaH (2.54 g, 63.44 mmol, 60% purity, 2.5 eq) in THE (50 mL) was added 4-bromo-1H-pyrrolo[2,3-b]pyridine (5 g, 25.38 mmol, 1 eq) and chlorotriisopropylsilane (7.34 g, 38.06 mmol, 8.15 mL, 1.5 eq). The mixture was stirred at 0° C. The mixture was stirred at 10° C. for 12 h. LCMS showed no starting material. TLC (Petroleum ether:EtOAc=5:1, Rf=1) showed one new spots was observed. The reaction mixture was quenched with 10 mL of saturated aqueous NH₄Cl. The mixture was concentrated in reduced pressure. The resulting solution was extracted with EtOAc (15 mL×3). The organic layers were dried over anhydrous Na₂SO₄ and concentrated under reduced pressure to give a residue. The residue was purified by column chromatography (SiO₂, Petroleum ether:EtOAc=1:0 to 3:1). The product (4-bromopyrrolo[2,3-b]pyridin-1-yl)-triisopropyl-silane (8.9 g, 22.67 mmol, 89.32% yield, 90% purity) was obtained as white solid. LCMS (ESI) m/z 355.0 [M+H]⁺

### (4-fluoropyrrolo[2,3-b]pyridin-1-yl)-triisopropyl-silane

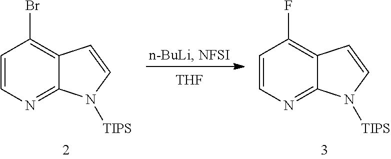

To a solution of (4-bromopyrrolo[2,3-b]pyridin-1-yl)-triisopropyl-silane (15 g, 42.45 mmol, 1 eq) in THF (150 mL) was added n-BuLi (2.5 M, 33.96 mL, 2 eq) at −78° C. under N₂. The mixture was stirred at −78° C. for 0.5 h under N₂. A solution of NFSI (20 g, 63.42 mmol, 1.49 eq) in THE (50 mL) was added at −78° C. The mixture was stirred at 10° C. for 11.5 h under N₂. LCMS showed no starting material. TLC (Petroleum ether/EtOAc=1:0, Rf=1) showed new spots was observed. The reaction was quenched with saturated aqueous NH₄Cl (30 mL). The mixture was concentrated in reduced pressure. The residue was diluted with H₂O (20 mL). The aqueous phase was extracted with EtOAc (50 mL×3) and washed with water (50 ml×3). The organic layers were dried over anhydrous Na₂SO₄ and concentrated under reduced pressure to give a residue. The residue was purified by column chromatography (SiO₂, Petroleum ether/EtOAc=1:0). The product (4-fluoropyrrolo[2,3-b]pyridin-1-yl)-triisopropyl-silane (9.3 g, 28.62 mmol, 67.42% yield, 90% purity) was obtained as yellow brown oil. LCMS (ESI) m/z 293.2 [M+H]⁺

### 4-fluoro-1H-pyrrolo[2,3-b]pyridine

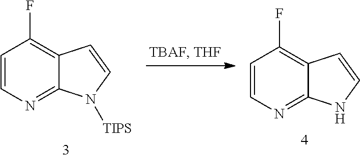

To a solution of (4-fluoropyrrolo[2,3-b]pyridin-1-yl)-triisopropyl-silane (17 g, 58.13 mmol, 1 eq) in THE (50 mL) was added TBAF (solution in THF) (1 M, 85.00 mL, 1.46 eq). The mixture was stirred at 10° C. for 2 h. LCMS showed no starting material. TLC (Petroleum ether/EtOAc=5:1, Rf=0.15) showed no starting material and new spots was observed. The mixture was concentrated in reduced pressure. The residue was diluted with EtOAc (30 mL). The aqueous phase was washed with H₂O (30 mL×3). The organic layers were dried over anhydrous Na₂SO₄ and concentrated under reduced pressure to give a residue. The residue was purified by column chromatography (SiO₂, Petroleum ether/EtOAc=1:0 to 3:1). The product 4-fluoro-1H-pyrrolo[2,3-b]pyridine (10 g, 55.10 mmol, 94.79% yield, 75% purity) was obtained as white solid. LCMS (ESI) m/z 137.0 [M+H]⁺

### 3-bromo-4-fluoro-1H-pyrrolo[2,3-b]pyridine

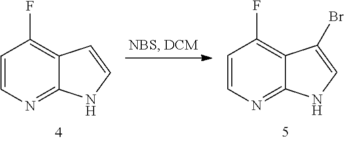

To a solution of 4-fluoro-1H-pyrrolo[2,3-b]pyridine (5 g, 36.73 mmol, 1 eq) in DCM (50 mL) was added a solution of NBS (8.50 g, 47.75 mmol, 1.3 eq) in DCM (50 mL) at 0° C. The mixture was stirred at 10° C. for 12 h. LCMS showed desired mass desired mass was detected. TLC (Petroleum ether/EtOAc=3:1, Rf=0.10) showed new spots was observed. The mixture was filtered and the filter cake was washed with 30 mL×3 of DCM. The aqueous phase was washed with 30 mL×3 of water. The organic layers were dried over anhydrous Na₂SO₄ and concentrated under reduced pressure to give a residue. The residue was purified by column chromatography (SiO₂, Petroleum ether/EtOAc=1:0 to 3:1). The product 3-bromo-4-fluoro-1H-pyrrolo[2,3-b]pyridine (9.7 g, 40.60 mmol, 55.27% yield, 90% purity) was obtained as yellow solid. LCMS (ESI) m/z 217.0 [M+H]⁺

### 3-bromo-4-fluoro-1H-pyrrolo[2,3-b]pyridine 7-oxide

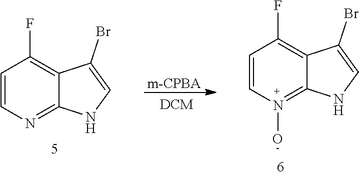

To a solution of 3-bromo-4-fluoro-1H-pyrrolo[2,3-b]pyridine (5 g, 23.25 mmol, 1 eq) in DCM (50 mL) was added a solution of m-CPBA (19 g, 93.59 mmol, 85% purity, 4.02 eq) in DCM (100 mL) at 0° C. The mixture was stirred at 10° C. for 12 h. LCMS showed no starting material desired mass was detected. The reaction was quenched with saturated aqueous Na₂SO₃ (30 mL). The mixture was filtered and the filter cake was wash with Na₂CO₃ (50 mL). The mixture was filtered and the filter was product 1. The crude product was used directly for the next step without purification. The product 1 3-bromo-4-fluoro-1H-pyrrolo[2,3-b]pyridine 7-oxide (8 g, 17.31 mmol, 37.23% yield, 50% purity) was obtained as yellow solid. LCMS (ESI) m/z [M+H]⁺

### methyl 3-bromo-6-chloro-4-fluoro-pyrrolo[2,3-b]pyridine-1-carboxylate

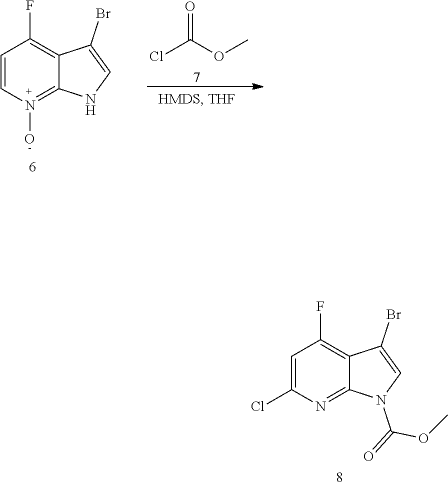

To a solution of 3-bromo-4-fluoro-7-oxido-1H-pyrrolo[2,3-b]pyridin-7-ium (7 g, 30.30 mmol, 1 eq) and HMDS (4.89 g, 30.30 mmol, 6.35 mL, 1 eq) in THE (100 mL) was added methyl carbonochloridate (8.59 g, 90.90 mmol, 7.04 mL, 3 eq) at 0° C. Then the mixture was stirred at 10° C. for 12 h. LCMS showed the starting material was consumed completely. The solvent was removed under reduced pressure and diluted with EtOAc (20 mL). Then the mixture was washed with NaHCO₃ (30 mL×3), the organic layers were dried over anhydrous Na₂SO₄ and concentrated under reduced pressure to give a residue. The crude product was used directly for the next step without purification. The product methyl 3-bromo-6-chloro-4-fluoro-pyrrolo[2,3-b]pyridine-1-carboxylate (5 g, crude) was obtained as white solid. LCMS (ESI) m/z 309.0 [M+H]⁺

### 3-bromo-6-chloro-4-fluoro-1H-pyrrolo[2,3-b]pyridine

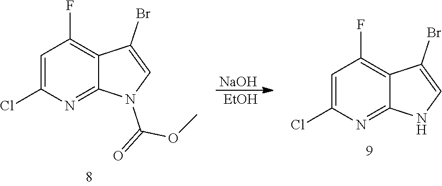

To a solution of methyl 3-bromo-6-chloro-4-fluoro-1H-pyrrolo[2,3-b]pyridine-1-carboxylate (5 g, 16.26 mmol, 1 eq) in MeOH (40 mL) was added NaOH (solved in water) (2 M, 27.03 mL, 3.32 eq). The mixture was stirred at 15° C. for 12 hr. LCMS showed the starting material was consumed completely. The mixture was diluted with EtOAc (30 mL), the organic phase was washed with saturated brine (30 mL×3), dried with anhydrous Na₂SO₄, filtered and concentrated in vacuo. The residue was purified by column chromatography (SiO₂, Petroleum ether:EtOAc=1:0 to 5:1). TLC (Petroleum ether:EtOAc=5:1, Rf=0.20) showed new spots was observed. The 3-bromo-6-chloro-4-fluoro-1H-pyrrolo[2,3-b]pyridine (2 g, 7.62 mmol, 46.84% yield, 95% purity) was obtained as white solid. LCMS (ESI) m/z 369.0 [M-TMS+H]⁺

### 3-bromo-6-chloro-4-fluoro-1-(p-tolylsulfonyl)pyrrolo[2,3-b]pyridine

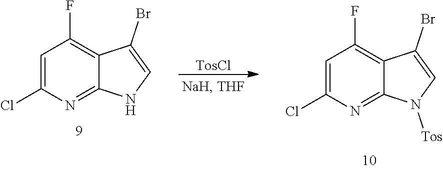

To a solution of 3-bromo-6-chloro-4-fluoro-1H-pyrrolo[2,3-b]pyridine (2.8 g, 11.22 mmol, 1 eq) in THE (40 mL) was added NaH (1.35 g, 33.67 mmol, 60% purity, 3 eq) at 0° C. TosCl (3.21 g, 16.84 mmol, 1.5 eq) was added. The mixture was stirred at 15° C. for 12 h. LCMS showed no starting material. TLC (Petroleum ether/EtOAc=10:1, Rf=0.50) showed no starting material and new spots was observed. The reaction mixture was quenched with 10 mL of saturated aqueous NH₄Cl. Then diluted with water (10 mL), acidified with HCl (2 M) to pH=6. The mixture was concentrated in reduced pressure. The resulting solution was extracted with EtOAc (30 mL×3). The organic layers were dried over anhydrous Na₂SO₄ and concentrated under reduced pressure to give a residue. The residue was purified by column chromatography (SiO₂, Petroleum ether/EtOAc=1:0 to 10:1). The product 3-bromo-6-chloro-4-fluoro-1-(p-tolylsulfonyl)pyrrolo[2,3-b]pyridine (4.16 g, 9.28 mmol, 82.64% yield, 90% purity) was obtained as white solid. LCMS (ESI) m/z 404.9 [M-TMS+H]⁺

### 4-fluoro-3,6-dimethyl-1-(p-tolylsulfonyl)pyrrolo[2,3-b]pyridine

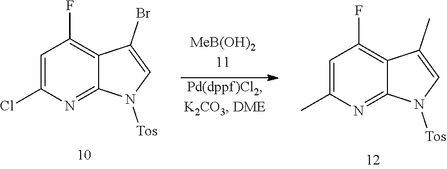

A mixture of 3-bromo-6-chloro-4-fluoro-1-(p-tolylsulfonyl)pyrrolo[2,3-b]pyridine (3.4 g, 8.42 mmol, 1 eq), MeB(OH)₂ (5.04 g, 84.23 mmol, 10 eq), K₂CO₃ (3.49 g, 25.27 mmol, 3 eq), Pd(dppf)Cl₂.CH₂Cl₂ (687.86 mg, 842.31 umol, 0.1 eq) in DMF (50 mL). Then the mixture was stirred at 120° C. for 12 hr under N₂. LCMS showed there were no starting material and main desired compound. The reaction mixture was added to water (100 mL). The resulting solution was extracted with EtOAc (30 mL×3). The organic layers were dried over anhydrous Na₂SO₄ and concentrated under reduced pressure to give a residue. The residue was purified by column chromatography (SiO₂, Petroleum ether/EtOAc=1:0 to 10:1). The product 4-fluoro-3,6-dimethyl-1-(p-tolylsulfonyl)pyrrolo[2,3-b]pyridine (1.2 g, 3.58 mmol, 42.51% yield, 95% purity) was obtained as a white solid. LCMS (ESI) m/z 318.9 [M+H]⁺

### 4-fluoro-3,6-dimethyl-1-(p-tolylsulfonyl)pyrrolo[2,3-b]pyridine-2-carboxylic acid

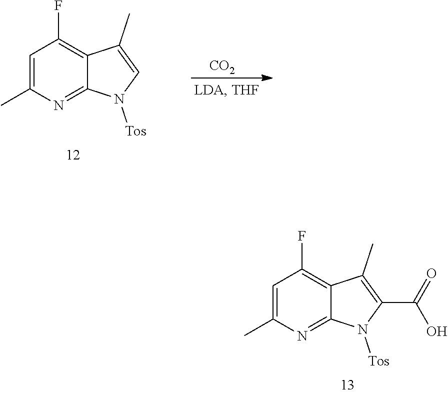

To a solution of 4-fluoro-3,6-dimethyl-1-(p-tolylsulfonyl)pyrrolo[2,3-b]pyridine (1.1 g, 3.46 mmol, 1 eq) in THE (10 mL) was added LDA (2 M, 3 mL, 1.74 eq) under N2 at −78° C. The mixture was stirred at −78° C. for 1.5 h. Then the mixture was stirred at −78° C. for 0.5 h under carbon dioxide (152.06 mg, 3.46 mmol, 1 eq). LC-MS showed 13% of the starting material was remained. The reaction was quenched with saturated aqueous NH₄Cl (2 mL). The mixture was filtered to give product 1. The aqueous phase was acidified with saturated aqueous Na₂CO₃ to pH=9. The mixture was washed with 20 mL×2 of EtOAc. The mixture was acidified with HCl (2 M) to pH=5. The mixture was filtered to give the product. The crude product was used directly for the next step without purification. The product 4-fluoro-3,6-dimethyl-1-(p-tolylsulfonyl)pyrrolo[2,3-b]pyridine-2-carboxylic acid (1.25 g, crude) was obtained as a yellow solid. LCMS (ESI) m/z 363.0 [M+H]⁺

### 4-fluoro-3,6-dimethyl-1H-pyrrolo[2,3-b]pyridine-2-carboxylic acid

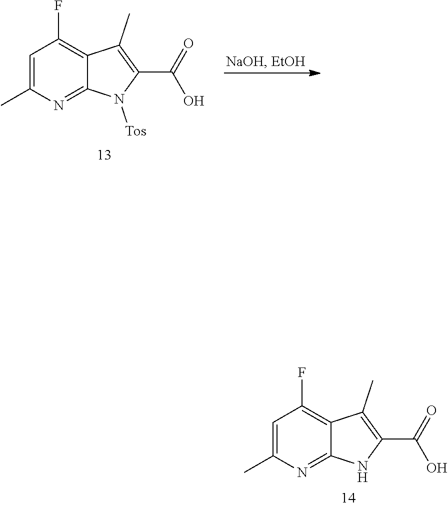

To a solution of 4-fluoro-3,6-dimethyl-1-(p-tolylsulfonyl)pyrrolo[2,3-b]pyridine-2-carboxylic acid (1 g, 2.76 mmol, 1 eq) in THE (10 mL) was added NaOH (solution in water) (2 M, 10 mL, 7.25 eq). The mixture was stirred at 30° C. for 12 h. LCMS showed no starting material and desired mass was detected. The mixture was acidified with HCl (2 M) to pH=8 and concentrated under reduced pressure. The mixture was washed with EtOAc (20 mL) and acidified with HCl (2 M) to pH=5. The mixture was filtered and the filter cake was washed with 10 mL×3 of Petroleum ether, dried under reduced pressure to give product. The residue was diluted in CH₃CN (5 mL) and H₂O (20 mL), then lyophilized. The product 4-fluoro-3,6-dimethyl-1H-pyrrolo[2,3-b]pyridine-2-carboxylic acid (360 mg, 1.64 mmol, 59.53% yield, 95% purity) was obtained as a white solid. LCMS (ESI) m/z 208.9 [M+H]⁺

### N-(1,1-dimethylsilinan-4-yl)-4-fluoro-3,6-dimethyl-1H-pyrrolo[2,3-b]pyridine-2-carboxamide

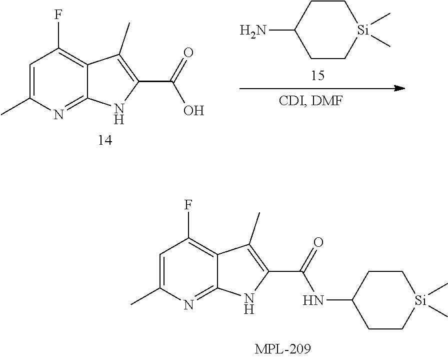

To a solution of 4-fluoro-3,6-dimethyl-1H-pyrrolo[2,3-b]pyridine-2-carboxylic acid (200 mg, 960.67 umol, 1 eq) and CDI (202.50 mg, 1.25 mmol, 1.3 eq) in DMF (2 mL). The mixture was stirred at 30° C. for 3 h. 1,1-dimethylsilinan-4-amine (178.96 mg, 1.25 mmol, 1.3 eq) was added. The mixture was stirred at 30° C. for 1 h. LC-MS showed the starting material was consumed completely. The reaction mixture was added to water (20 mL), then filtered and the filter cake was washed with 10 mL of water, dried in vacuo to give product. The crude product was purified by prep-HPLC (column: YMC-Actus Triart C18 100*30 mm*5 um; mobile phase: [water (0.225% FA)-ACN]; B %: 44%-74%, 11 min). Then lyophilized. The product N-(1,1-dimethylsilinan-4-yl)-4-fluoro-3,6-dimethyl-1H-pyrrolo[2,3-b]pyridine-2-carboxamide (30.1 mg, 89.64 umol, 9.33% yield, 99.315% purity) was obtained as a yellow solid.

LCMS (ESI) m/z 334.1 [M+H]⁺; ¹H NMR (500 MHz, DMSO-d₆) δ=11.83 (br s, 1H), 7.75 (br d, J=7.6 Hz, 1H), 6.81 (d, J=11.9 Hz, 1H), 3.76-3.65 (m, 1H), 2.56 (s, 3H), 2.51 (br s, 3H), 2.05-1.96 (m, 2H), 1.63-1.52 (m, 2H), 0.78 (br d, J=14.6 Hz, 2H), 0.60 (dt, J=4.6, 13.7 Hz, 2H), 0.08 (s, 3H), 0.03 (s, 3H).

### Example 26. MPL-210

### Synthesis of N-(1,1-dimethylsilinan-4-yl)-4-fluoro-3-methyl-1H-pyrrolo[2,3-b]pyridine-2-carboxamide

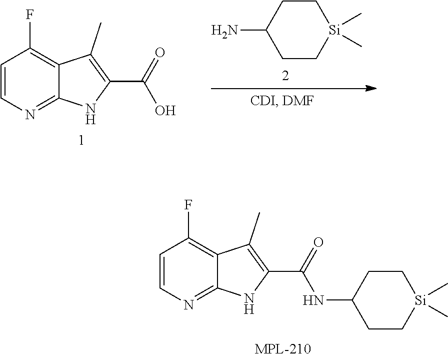

To a solution of 4-fluoro-3-methyl-1H-pyrrolo[2,3-b]pyridine-2-carboxylic acid (290 mg, 1.49 mmol, 1 eq) in DMF (5 mL) was added CDI (290.62 mg, 1.79 mmol, 1.2 eq). The mixture was stirred at 30° C. for 0.5 hr. Then 1,1-dimethylsilinan-4-amine (235.44 mg, 1.64 mmol, 1.1 eq) was added, the mixture was stirred at 30° C. further 1 hr. LC-MS showed reactant was consumed completely and desired mass was detected. The mixture was filtered; the filtrate was purified by prep-HPLC (column: YMC-Actus Triart C18 150*30 mm*5 um; mobile phase: [water (0.225% FA)-ACN]; B %: 58%-88%, 10 min). LCMS showed the product was not pure enough after prep-HPLC, the product was then washed with MeCN (10 mL). Filtered, the filter cake was combined with dried in lyophilizer. Compound N-(1,1-dimethylsilinan-4-yl)-4-fluoro-3-methyl-1H-pyrrolo[2,3-b]pyridine-2-carboxamide (70 mg, 219.13 umol, 14.67% yield, 100% purity) was obtained as a white solid.

LCMS (ESI) m/z 320.0 [M+H]⁺; ¹H NMR (500 MHz, DMSO-d₆) δ=11.93 (br s, 1H) 8.21 (dd, J=7.86, 5.57 Hz, 1H) 7.79 (br d, J=7.63 Hz, 1H) 6.85 (dd, J=10.91, 5.26 Hz, 1H) 3.57-3.69 (m, 1H) 2.51 (s, 3H) 1.89-1.99 (m, 2H) 1.42-1.56 (m, 2H) 0.70 (br d, J=14.65 Hz, 2H) 0.52 (td, J=13.73, 4.58 Hz, 2H) −0.10-0.03 (m, 6H).

### Example 27. MPL-213

### Synthesis of 4-fluoro-N-[(1R,2R,3S,5R)-2-hydroxy-2,6,6-trimethyl-norpinan-3-yl]-1H-pyrrolo[2,3-b]pyridine-2-carboxamide

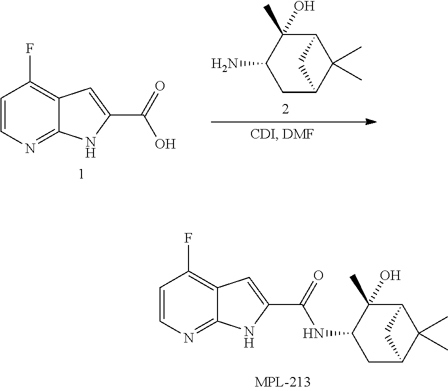 [SMILES: O=C(O)c1cc2c(F)ccnc2[nH]1 ; CC1(C)[C@@H]2C[C@H]1[C@@](C)(O)[C@@H](N)C2 ; CC1(C)[C@H]2C[C@H](NC(=O)c3cc4c(F)ccnc4[nH]3)[C@](C)(O)[C@@H]1C2]

To a solution of 4-fluoro-1H-pyrrolo[2,3-b]pyridine-2-carboxylic acid (50 mg, 277.57 umol, 1 eq) in DMF (2 mL) was added CDI (54.01 mg, 333.08 umol, 1.2 eq). The mixture was stirred at 30° C. for 0.5 hr. Then (1R,2R,3S,5R)-3-amino-2,6,6-trimethyl-norpinan-2-ol (65.77 mg, 388.60 umol, 1.4 eq) was added. The mixture was stirred at 30° C. further 12 hr. LCMS showed reactant was consumed completely and one main peak with desired mass was detected. The mixture was filtered; the filtrate was purified by prep-HPLC (column: YMC-Actus Triart C18 100*30 mm*5 um; mobile phase: [water (0.225% FA)-ACN]; B %: 44%-64%, 11 min). Compound 4-fluoro-N-[(1R,2R,3S,5R)-2-hydroxy-2,6,6-trimethyl-norpinan-3-yl]-1H-pyrrolo[2,3-b]pyridine-2-carboxamide (53 mg, 155.36 umol, 55.97% yield, 97.138% purity) was obtained as a white solid.

LCMS (ESI) m/z 332.2 [M+H]⁺; ¹H NMR (400 MHz, DMSO-d₆) δ=ppm 12.55 (br s, 1H), 8.32 (dd, J=8.22, 5.48 Hz, 1H), 8.02 (d, J=8.61 Hz, 1H), 7.26 (d, J=1.96 Hz, 1H), 7.01 (dd, J=10.37, 5.28 Hz, 1H), 4.44-4.60 (m, 2H), 2.21-2.30 (m, 1H), 2.07-2.16 (m, 1H), 1.89 (br d, J=5.48 Hz, 2H), 1.57-1.70 (m, 2H), 1.26 (s, 3H), 1.20 (s, 3H), 1.06 (s, 3H).

### Example 28. MPL-216

### Synthesis of N-[(1R,2R,3S,5R)-2-hydroxy-2,6,6-trimethyl-norpinan-3-yl]-4-(trifluoro methyl)-1H-pyrrolo[2,3-b]pyridine-2-carboxamide

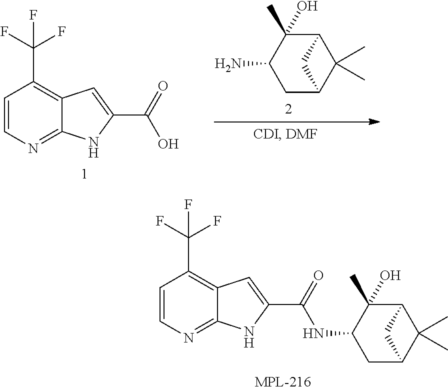 [SMILES: O=C(O)c1cc2c(C(F)(F)F)ccnc2[nH]1 ; CC1(C)[C@@H]2C[C@H]1[C@@](C)(O)[C@@H](N)C2 ; CC1(C)[C@H]2C[C@H](NC(=O)c3cc4c(C(F)(F)F)ccnc4[nH]3)[C@](C)(O)[C@@H]1C2]

To a solution of 4-(trifluoromethyl)-1H-pyrrolo[2, 3-b]pyridine-2-carboxylic acid (150 mg, 651.77 umol, 1 eq) in DMF (4 mL) was added CDI (116.25 mg, 716.94 umol, 1.1 eq). The mixture was stirred at 30° C. for 0.5 h. Then (1R,2R, 3S, 5R)-3-amino-2, 6, 6-trimethyl-norpinan-2-ol (143.42 mg, 847.30 umol, 1.3 eq) was added. The mixture was stirred at 30° C. for 11.5 h. LCMS showed there were main desired compound and a little starting material. Then (1R,2R, 3S, 5R)-3-amino-2, 6, 6-trimethyl-norpinan-2-ol (0.2 eq, 22 mg) was added. The mixture was stirred at 30° C. for 2 h. LCMS showed there were main desired compound and a little starting material. The reaction was added dropwise to H₂O (20 mL). There was much precipitation which was collected by filter. The cake was transferred in bottom flask. The residue was purified by perp. HPLC (column: YMC-Actus Triart C18 100*30 mm*5 um; mobile phase: [water (0.225% FA)-ACN]; B %: 50%-79%, 11 min). Compound N-[(1R,2R, 3S, 5R)-2-hydroxy-2, 6, 6-trimethyl-norpinan-3-yl]-4-(trifluoromethyl)-1H-pyrrolo[2,3-b]pyridine-2-carboxamide (88.2 mg, 223.01 umol, 34.22% yield, 96.434% purity) was obtained as a white solid.

LCMS (ESI), m/z 382.2[M+H]⁺; ¹H NMR (400 MHz, DMSO-d6) δ=12.81 (br s, 1H), 8.55 (d, J=4.3 Hz, 1H), 8.23 (d, J=9.0 Hz, 1H), 7.49 (d, J=5.1 Hz, 1H), 7.40 (s, 1H), 4.62-4.54 (m, 1H), 4.53 (s, 1H), 2.26 (br t, J=10.8 Hz, 1H), 2.12 (br s, 1H), 1.90 (br d, J=5.1 Hz, 2H), 1.71 (br dd, J=8.0, 13.5 Hz, 1H), 1.64 (d, J=9.8 Hz, 1H), 1.27 (s, 3H), 1.22 (s, 3H), 1.07 (s, 3H).

### Example 29. MPL-218

### Synthesis of 4-chloro-N-[(1R,2R,3S,5R)-2-hydroxy-2,6,6-trimethyl-norpinan-3-yl]-6-methyl-1H-pyrrolo[2,3-b]pyridine-2-carboxamide

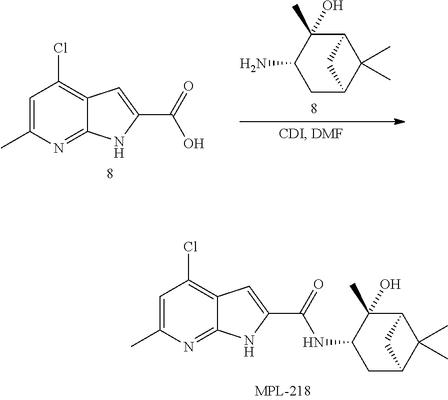

To a solution of 4-chloro-6-methyl-1H-pyrrolo[2,3-b]pyridine-2-carboxylic acid (50 mg, 237.40 umol, 1 eq) in DMF (2 mL) was added CDI (50.04 mg, 308.62 umol, 1.3 eq). The mixture was stirred at 30° C. for 0.5 hr. Then (1R,2R,3S,5R)-3-amino-2,6,6-trimethyl-norpinan-2-ol (56.26 mg, 332.36 umol, 1.4 eq) was added. The mixture was stirred at 30° C. further 12 hr. LCMS showed reactant was consumed completely and one main peak with desired mass was detected. The mixture was filtered; the filtrate was purified by prep-HPLC column: YMC-Actus Triart C18 100*30 mm*5 um; mobile phase: [water (0.225% FA)-ACN]; B %: 53%-78%, 11 min. Compound 4-chloro-N-[(1R,2R,3S,5R)-2-hydroxy-2,6,6-trimethyl-norpinan-3-yl]-6-methyl-1H-pyrrolo[2,3-b]pyridine-2-carboxamide (44 mg, 115.14 umol, 48.50% yield, 94.693% purity) was obtained as a white solid.

LCMS (ESI) m/z 332.2 [M+H]⁺; ¹H NMR (400 MHz, DMSO-d₆) δ=12.36 (s, 1H), 8.04 (d, J=9.16 Hz, 1H), 7.24 (d, J=2.14 Hz, 1H), 7.19 (s, 1H), 4.44-4.63 (m, 2H), 2.55 (s, 3H), 2.27 (br t, J=11.14 Hz, 1H), 2.10-2.17 (m, 1H), 1.91 (br d, J=5.65 Hz, 2H), 1.69 (dd, J=13.35, 7.55 Hz, 1H), 1.63 (d, J=9.92 Hz, 1H), 1.28 (s, 3H), 1.23 (s, 3H), 1.08 (s, 3H).

### Example 30. MPL-219

### 4,5-difluoro-N-[(1R,2R,3S,5R)-2-hydroxy-2,6,6-trimethyl-norpinan-3-yl]-1H-pyrrolo[2,3-b]pyridine-2-carboxamide

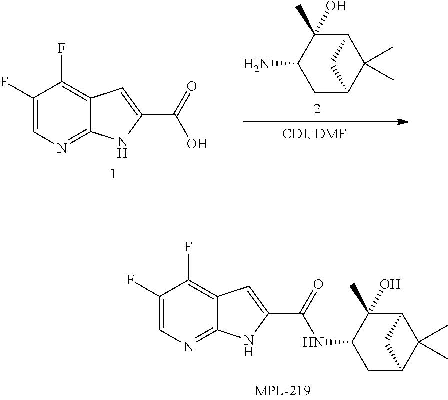 [SMILES: O=C(O)c1cc2c(F)c(F)cnc2[nH]1 ; CC1(C)[C@@H]2C[C@H]1[C@@](C)(O)[C@@H](N)C2 ; CC1(C)[C@H]2C[C@H](NC(=O)c3cc4c(F)c(F)cnc4[nH]3)[C@](C)(O)[C@@H]1C2]

To a solution of 4,5-difluoro-1H-pyrrolo[2,3-b]pyridine-2-carboxylic acid (90 mg, 454.26 umol, 1 eq) and CDI (88.39 mg, 545.11 umol, 1.2 eq) in DMF (2 mL). The mixture was stirred at 30° C. for 3 h. (1R,2R,3S,5R)-3-amino-2,6,6-trimethyl-norpinan-2-ol (92.27 mg, 545.11 umol, 1.2 eq) was added. The mixture was stirred at 30° C. for 1 h. LC-MS showed the starting material was consumed completely. The reaction mixture was added to water (20 mL), then filtered and the filter cake was washed with 10 mL of water, dried in vacuo to give product. The crude product was purified by prep-HPLC (column: YMC-Actus Triart C18 100*30 mm*5 um; mobile phase: [water (0.225% FA)-ACN]; B %: 43%-72%, 11 min), then lyophilized. The product 4,5-difluoro-N-[(1R,2R,3 S,5R)-2-hydroxy-2,6,6-trimethyl-norpinan-3-yl]-1H-pyrrolo[2,3-b]pyridine-2-carboxamide (48.2 mg, 135.85 umol, 29.91% yield, 98.468% purity) was obtained as a yellow solid.

LCMS (ESI) m/z 349.9 [M+H]⁺; ¹H NMR (500 MHz, DMSO-d₆) δ=12.68 (br s, 1H), 8.50 (dd, J=3.4, 9.8 Hz, 1H), 8.07 (d, J=9.0 Hz, 1H), 7.34 (s, 1H), 4.57-4.50 (m, 2H), 2.28 (br t, J=11.2 Hz, 1H), 2.16-2.09 (m, 1H), 1.90 (br d, J=5.8 Hz, 2H), 1.70-1.59 (m, 2H), 1.27 (s, 3H), 1.21 (s, 3H), 1.07 (s, 3H).

### Example 31. MPL-221

### 4-fluoro-N-[(1R,2R,3S,5R)-2-hydroxy-2,6,6-trimethyl-norpinan-3-yl]-1H-pyrrolo[2,3-c]pyridine-2-carboxamide

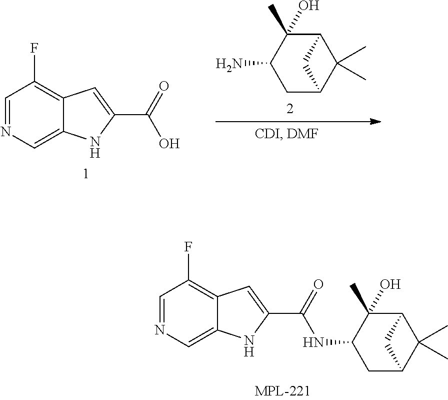 [SMILES: O=C(O)c1cc2c(F)cncc2[nH]1 ; CC1(C)[C@@H]2C[C@H]1[C@@](C)(O)[C@@H](N)C2 ; CC1(C)[C@H]2C[C@H](NC(=O)c3cc4c(F)cncc4[nH]3)[C@](C)(O)[C@@H]1C2]

To a solution of 4-fluoro-1H-pyrrolo[2,3-c]pyridine-2-carboxylic acid (100 mg, 555.14 umol, 1 eq) and CDI (117.02 mg, 721.68 umol, 1.3 eq) in DMF (1.5 mL). The mixture was stirred at 30° C. for 0.5 h. Then (1R,2R,3S,5R)-3-amino-2,6,6-trimethyl-norpinan-2-ol (122.15 mg, 721.68 umol, 1.3 eq) was added. The mixture was stirred at 30° C. for 11.5 h. LC-MS showed most of the starting material was consumed. The reaction mixture was added to water (20 mL), then filtered and the filter cake was washed with 10 mL of water, dried in vacuo to give product. The residue was diluted in CH₃CN (5 mL) and H₂O (20 mL), then lyophilized. The product 4-fluoro-N-[(1R,2R,3S,5R)-2-hydroxy-2,6,6-trimethyl-norpinan-3-yl]-1H-pyrrolo[2,3-c]pyridine-2-carboxamide (61.1 mg, 181.29 umol, 32.66% yield, 98.326% purity) was obtained as a white solid.

LCMS (ESI) m/z 332.2 [M+H]⁺; ¹H NMR (400 MHz, DMSO-d₆) δ=12.47 (br s, 1H), 8.67 (d, J=2.4 Hz, 1H), 8.19 (br d, J=8.8 Hz, 1H), 8.09 (d, J=1.7 Hz, 1H), 7.41 (s, 1H), 4.61-4.49 (m, 2H), 2.34-2.25 (m, 1H), 2.16-2.09 (m, 1H), 1.90 (br d, J=5.4 Hz, 2H), 1.75-1.60 (m, 2H), 1.27 (s, 3H), 1.23 (s, 3H), 1.07 (s, 3H).

### Example 32. MPL-222

### Synthesis of 4-chloro-N-[(1R,2R, 3S, 5R)-2-hydroxy-2,6,6-trimethyl-norpinan-3-yl]-1H-Pyrrolo[2,3-c]pyridine-2-carboxamide

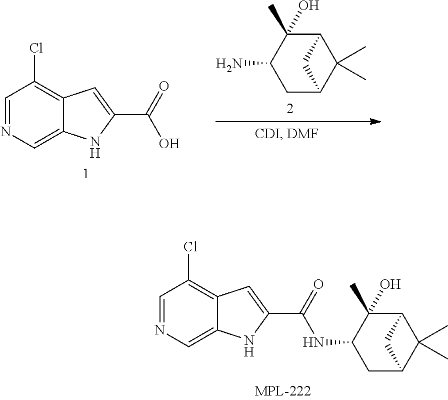 [SMILES: O=C(O)c1cc2c(Cl)cncc2[nH]1 ; CC1(C)[C@@H]2C[C@H]1[C@@](C)(O)[C@@H](N)C2 ; CC1(C)[C@H]2C[C@H](NC(=O)c3cc4c(Cl)cncc4[nH]3)[C@](C)(O)[C@@H]1C2]

To a solution of 4-chloro-1H-pyrrolo[2, 3-c]pyridine-2-carboxylic acid (50 mg, 254.34 umol, 1 eq) DMF (1.5 mL) was added CDI (45.36 mg, 279.77 umol, 1.1 eq). The mixture was stirred at 30° C. for 0.5 h. Then (1R,2R, 3S, 5R)-3-amino-2, 6, 6-trimethyl-norpinan-2-ol (45.20 mg, 267.05 umol, 1.05 eq) was added. The mixture was stirred at 30° C. for 11.5 h. LCMS showed there was no starting material. The reaction was added dropwise to H₂O (20 mL). There was much precipitation which was collected by filter. The cake was transferred in bottom flask. The residue was purified by prep-HPLC (column: YMC-Actus Triart C18 100*30 mm*5 um; mobile phase: [water (0.225% F A)-ACN]; B %:25%-55%, 11 min) Compound 4-chloro-N-[(1R,2R,3S,5R)-2-hydroxy-2, 6, 6-trimethyl-norpinan-3-yl]-1H-pyrrolo[2,3-c]pyridine-2-carboxamide (33 mg, 94.72 umol, 37.24% yield, 99.840% purity) was obtained as a white solid.

LCMS (ESI), m/z 348.0[M+H]⁺

¹H NMR (500 MHz, CDCl3) δ=10.58 (br s, 1H), 8.85 (s, 1H), 8.28 (s, 1H), 7.53 (br d, J=7.5 Hz, 1H), 7.01 (s, 1H), 4.61-4.55 (m, 1H), 2.78-2.71 (m, 1H), 2.37-2.28 (m, 1H), 2.11-2.05 (m, 2H), 1.68 (br dd, J=6.2, 13.0 Hz, 1H), 1.49 (d, J=10.5 Hz, 1H), 1.41 (s, 3H), 1.35 (s, 3H), 1.16 (s, 3H).

### Example 33. MPL-223

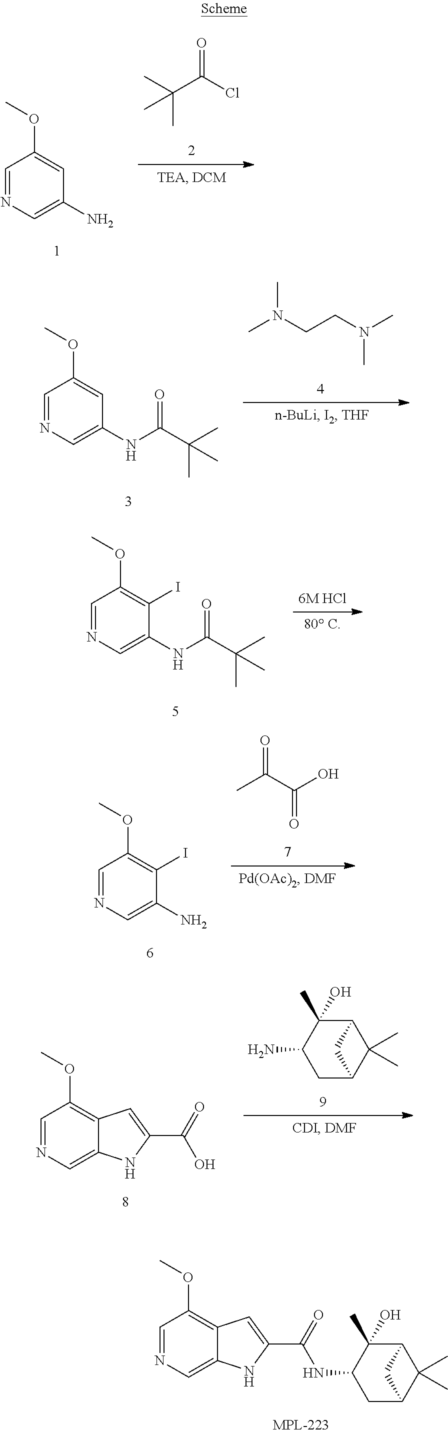

### Synthesis of N-(5-methoxy-3-pyridyl)-2,2-dimethyl-propanamide

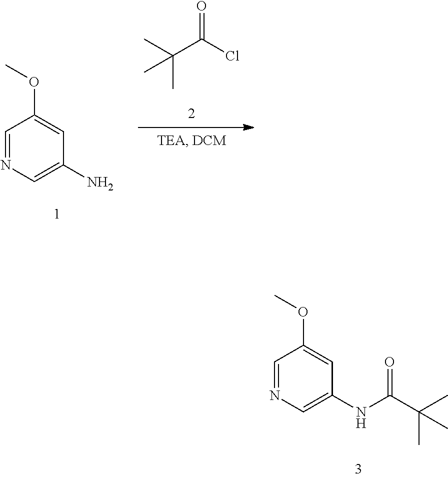

To an ice-cooled solution of 5-methoxypyridin-3-amine (10 g, 80.55 mmol, 1 eq) in CH₂Cl₂ (100 mL) was added TEA (24.45 g, 241.66 mmol, 33.64 mL, 3 eq). Then 2,2-dimethylpropanoyl chloride (10.68 g, 88.61 mmol, 10.90 mL, 1.1 eq) was added at 0° C. The mixture was allowed warm to 10° C. gradually and stirred 12 hr. TLC (Petroleum ether:EtOAc=1:1, Rf=0.3) indicated the starting material was consumed completely, and one major new sport with lower polarity was detected. Water (100 mL) was added to the mixture, followed by CH₂Cl₂ (200 mL). The separated organic layer was washed with brine (Sat. 200 mL) then dried over Na₂SO₄, filtered and concentrated under reduced pressure to afford crude product which was purified by flash silica gel chromatography (ISCO®; 80 g SepaFlash® Silica Flash Column, Eluent of 0˜80% EtOAc/Petroleum ether gradient at 60 mL/min). Compound N-(5-methoxy-3-pyridyl)-2,2-dimethyl-propanamide (15 g, 68.43 mmol, 84.94% yield, 95% purity) was obtained as a white solid.

### Synthesis of N-(4-iodo-5-methoxy-3-pyridyl)-2,2-dimethyl-propanamide

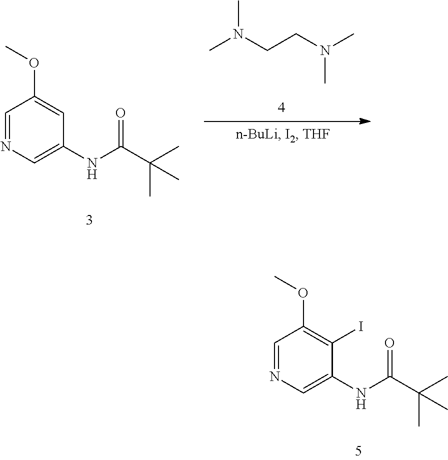

To a solution of N-(5-methoxy-3-pyridyl)-2,2-dimethyl-propanamide (14 g, 67.22 mmol, 1 eq) in dried THE (150 mL) was added TMEDA (25.00 g, 215.12 mmol, 32.46 mL, 3.2 eq). The solution was cooled to −78° C. then n-BuLi (2.5 M, 86.05 mL, 3.2 eq) was added under N2 (maintain temperature below −60° C.). The mixture was stirred at −78° C. for 3 hr. I₂ (27.30 g, 107.56 mmol, 21.67 mL, 1.6 eq) in dried THE (80 mL) was added dropwise (maintain temperature below −60° C.). The mixture was allowed warm to 10° C. after addition and stirred further 12 hr. TLC (Petroleum ether:EtOAc=1:1, Rf=0.4) indicated starting material was consumed completely and one new spot formed. Na₂SO₃ (Sat. in water, 100 mL) was added to the mixture, THE was removed under reduced pressure. The product was extracted with CH₂Cl₂ (150 mL×3), the combined organic layer was dried over Na₂SO₄. Filtered, the filtrate was concentrated under reduced pressure at 40° C. until 100 mL solvent left. The product was recrystallized in CH₂Cl₂, filtered to give the product. Compound N-(4-iodo-5-methoxy-3-pyridyl)-2,2-dimethyl-propanamide (19.2 g, 54.59 mmol, 81.20% yield, 95% purity) was obtained as a pink solid.

### Synthesis of 4-iodo-5-methoxy-pyridin-3-amine

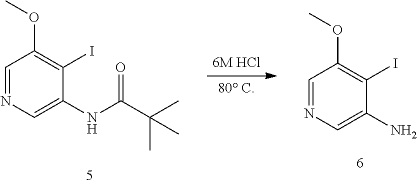

The reactant N-(4-iodo-5-methoxy-3-pyridyl)-2,2-dimethyl-propanamide (12 g, 35.91 mmol, 1 eq) was dissolved in HCl (6 M, 150 mL, 25.06 eq). The mixture was stirred at 80° C. for 12 hr. TLC (Petroleum ether:EtOAc=1:1, Rf=0.3) indicated the starting material was consumed completely and one new spot with larger polarity was detected. NaOH (6M, in water) was added to adjust pH to 8. The product was extracted with EtOAc (50 mL×4), the combined organic layer was washed with brine (50 mL), dried over Na₂SO₄. Filtered and concentrated under reduced pressure to afford the product. Compound 4-iodo-5-methoxy-pyridin-3-amine (8.4 g, 31.92 mmol, 88.87% yield, 95% purity) was obtained as a yellow solid.

### Synthesis of 4-methoxy-1H-pyrrolo[2,3-c]pyridine-2-carboxylic acid

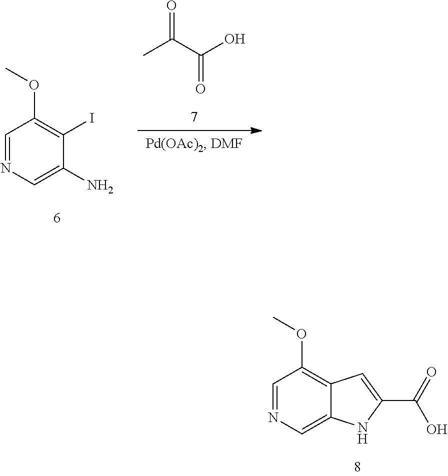

To a mixture of 4-iodo-5-methoxy-pyridin-3-amine (3 g, 12.00 mmol, 1 eq), 2-oxopropanoic acid (2.26 g, 18.00 mmol, 1.81 mL, 1.5 eq) and DABCO (2.69 g, 24.00 mmol, 2.64 mL, 2 eq) was added DMF (100 mL). Then Pd(OAc)₂ (538.74 mg, 2.40 mmol, 0.2 eq) was added under N₂. The mixture was stirred at 115° C. for 4 hr. LCMS showed Reactant was consumed completely and one main peak with desired mass was detected. DMF was removed under reduced pressure by oil pump. Toluene (60 mL) was added to the mixture and washed in ultrasound for 15 min. Then toluene was pooled off carefully while the brown solid was remained. The solid was redissolved in water (50 mL), the turbid liquid was washed in ultrasound while HCl (6M, in water) was added dropwise to adjust pH to 5. Filtered, the filter cake was washed with MeCN (20 mL) in ultrasound (30 min), filtered to afford the product. Compound 4-methoxy-1H-pyrrolo[2,3-c]pyridine-2-carboxylic acid (2.03 g, 10.04 mmol, 83.64% yield, 95% purity) was obtained as a brown solid. LCMS (ESI) m/z 193.1 [M+H]⁺

### Synthesis of N-[(1R,2R,3S,5R)-2-hydroxy-2,6,6-trimethyl-norpinan-3-yl]-4-methoxy-1H-pyrrolo[2,3-c]pyridine-2-carboxamide

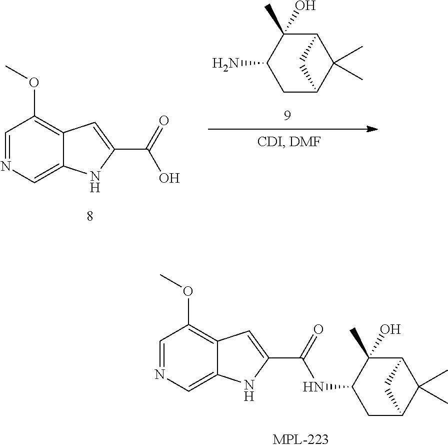

To a solution of 4-methoxy-1H-pyrrolo[2,3-c]pyridine-2-carboxylic acid (50 mg, 260.18 umol, 1 eq) in DMF (1 mL) was added CDI (54.85 mg, 338.24 umol, 1.3 eq). The mixture was stirred at 30° C. for 0.5 hr. Then (1R,2R,3S,5R)-3-amino-2,6,6-trimethyl-norpinan-2-ol (57.25 mg, 338.24 umol, 1.3 eq) was added, the mixture was stirred at 30° C. for 1 hr. LCMS showed reactant was consumed completely and one main peak with desired mass was detected. The mixture was filtered, the filtrate was purified by prep-HPLC (column: YMC-Actus Triart C18 100*30 mm*5 um; mobile phase: [water (0.225% FA)-ACN]; B %: 27%-57%, 11 min). Compound N-[(1R,2R,3S,5R)-2-hydroxy-2,6,6-trimethyl-norpinan-3-yl]-4-methoxy-1H-pyrrolo[2,3-c]pyridine-2-carboxamide (88 mg, 244.89 umol, 94.12% yield, 95.569% purity) was obtained as a white solid.

LCMS (ESI) m/z 344.2 [M+H]⁺; ¹H NMR (500 MHz, DMSO-d₆) δ=12.48 (br s, 1H), 8.57 (br s, 1H), 8.18 (br d, J=9.00 Hz, 1H), 7.89 (br s, 1H), 7.45 (s, 1H), 4.48-4.61 (m, 2H), 4.04 (s, 3H), 2.25-2.33 (m, 1H), 2.10-2.18 (m, 1H), 1.91 (br d, J=5.80 Hz, 2H), 1.71 (br dd, J=13.43, 7.48 Hz, 1H), 1.64 (d, J=9.92 Hz, 1H), 1.28 (s, 3H) 1.26-1.26 (m, 1H), 1.24 (s, 3H), 1.08 (s, 3H).

### Example 34. MPL-226

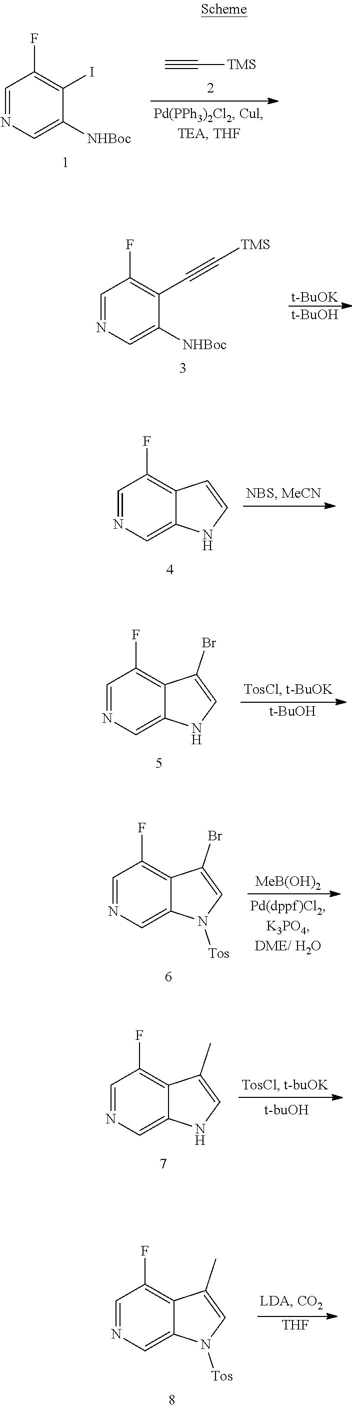

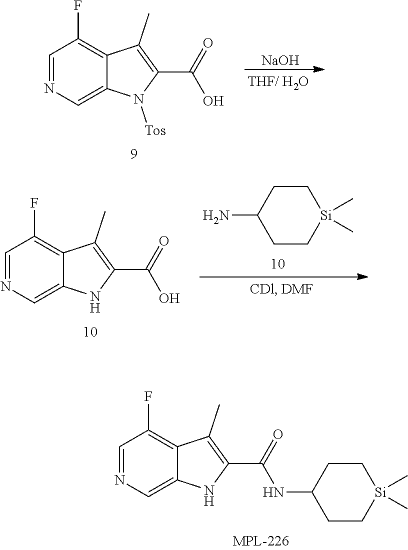

### tert-butyl N-[5-fluoro-4-(2-trimethylsilylethynyl)-3-pyridyl]carbamate

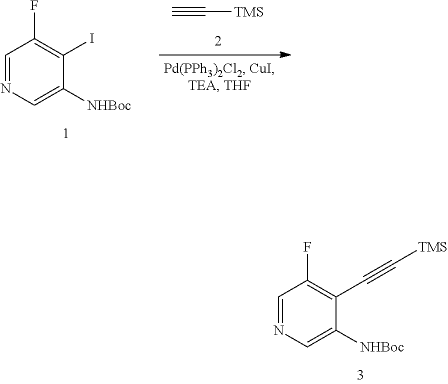

To a solution of tert-butyl N-(5-fluoro-4-iodo-3-pyridyl)carbamate (20 g, 59.15 mmol, 1 eq) Pd(PPh₃)₂Cl₂ (2.08 g, 2.96 mmol, 0.05 eq), CuI (3.38 g, 17.75 mmol, 0.3 eq) in THE (200 mL) was added ethynyl(trimethyl)silane (58.10 g, 591.51 mmol, 81.94 mL, 10 eq) and TEA (17.96 g, 177.45 mmol, 24.70 mL, 3 eq) under N₂. The mixture was stirred at 30° C. for 5 hr under N₂. LCMS showed 8% of the starting material still remaining and the desired compound as the main product. The mixture was concentrated in reduced pressure. The residue was diluted with H₂O (100 mL). The aqueous phase was extracted with EtOAc (100 mL×3). The combined organic phase was washed with saturated NaCl (100 mL×2). The organic layers were dried over anhydrous Na₂SO₄ and concentrated under reduced pressure to give a residue. The residue was purified by column chromatography (SiO₂, Petroleum ether/EtOAc=1:0 to 10:1). The product tert-butyl N-[5-fluoro-4-(2-trimethylsilylethynyl)-3-pyridyl]carbamate (16 g, 51.88 mmol, 87.70% yield) was obtained as a yellow solid. LCMS (ESI) m/z 309.1 [M+H]⁺

### 4-fluoro-1H-pyrrolo[2,3-c]pyridine

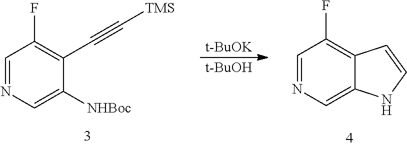 [SMILES: "t-BuOK" ; "t-BuOH" ; CC(C)(C)OC(=O)Nc1cncc(F)c1C#C[Si](C)(C)C ; Fc1cncc2[nH]ccc12]

To a solution of tert-butyl N-[5-fluoro-4-(2-trimethylsilylethynyl)-3-pyridyl]carbamate (16 g, 51.88 mmol, 1 eq) in t-BuOH (200 mL) was added t-BuOK (17.46 g, 155.63 mmol, 3 eq). The mixture was stirred at 80° C. for 9 h. LCMS showed desired compound mass was detected. TLC (Petroleum ether:EtOAc=1:1) showed most of the starting material 1 was consumed and new spots was observed. The mixture was concentrated in reduced pressure. The mixture was diluted with EtOAc (50 mL). The filtrate was washed with water (50 mL×3). The organic layers were dried over anhydrous Na₂SO₄ and concentrated under reduced pressure to give a residue. The residue was purified by column chromatography (SiO₂, Petroleum ether/EtOAc=1:0 to 1:1). The crude product 4-fluoro-1H-pyrrolo[2,3-c]pyridine (4.7 g, 32.80 mmol, 63.23% yield, 95% purity) was obtained as a yellow solid. LCMS (ESI) m/z [M+H]⁺

### 3-bromo-4-fluoro-1H-pyrrolo[2,3-c]pyridine

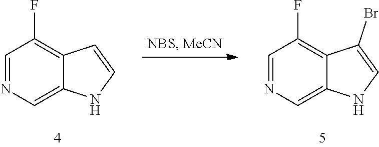 [SMILES: Fc1cncc2[nH]ccc12 ; Fc1cncc2[nH]cc(Br)c12]

To a solution of 4-fluoro-1H-pyrrolo[2,3-c]pyridine (4.2 g, 30.85 mmol, 1 eq) in MeCN (50 mL) was added NBS (8 g, 44.95 mmol, 1.46 eq) at 0° C. The mixture was stirred at 30° C. for 12 h. LCMS showed desired mass was detected. TLC (Petroleum ether/EtOAc=3:1, Rf=0.10) showed new spots was observed. The crude product was used directly for the next step without purification. The product 3-bromo-4-fluoro-1H-pyrrolo[2,3-c]pyridine (6 g, crude) was obtained as yellow solid.

LCMS (ESI) m/z 216.9 [M+H]⁺

### 3-bromo-4-fluoro-1-(p-tolylsulfonyl)pyrrolo[2,3-c]pyridine

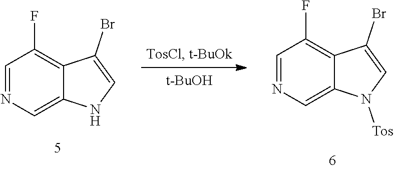

To a solution of 3-bromo-4-fluoro-1H-pyrrolo[2,3-c]pyridine (6 g, 27.90 mmol, 1 eq) in t-BuOH (10 mL) was added t-BuOK (9.39 g, 83.71 mmol, 3 eq) and TosCl (6.92 g, 36.28 mmol, 1.3 eq). The mixture was stirred at 25° C. for 12 h. LC-MS showed the starting material was consumed completely. The mixture was concentrated in reduced pressure. The mixture was diluted with EtOAc (50 mL). The filtrate was washed with water (50 mL×3). The organic layers were dried over anhydrous Na₂SO₄ and concentrated under reduced pressure to give a residue. The residue was purified by column chromatography (SiO₂, Petroleum ether/EtOAc=1:0 to 5:1). The product 3-bromo-4-fluoro-1-(p-tolylsulfonyl)pyrrolo[2,3-c]pyridine (3.8 g, 9.78 mmol, 35.04% yield, 95% purity) was obtained as a yellow solid. LCMS (ESI) m/z 369.0 [M-TMS+H]⁺

### 4-fluoro-3-methyl-1H-pyrrolo[2,3-c]pyridine

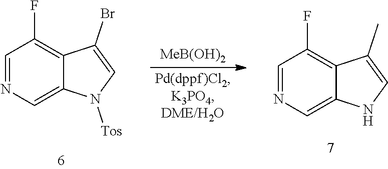

A mixture of 3-bromo-4-fluoro-1-(p-tolylsulfonyl)pyrrolo[2,3-c]pyridine (3.8 g, 10.29 mmol, 1 eq), MeB(OH)₂ (6.16 g, 102.92 mmol, 10 eq), Pd(dppf)Cl₂.CH₂Cl₂ (840.51 mg, 1.03 mmol, 0.1 eq), K₂CO₃ (4.27 g, 30.88 mmol, 3 eq) in DMF (40 mL). Then the mixture was stirred at 120° C. for 12 hr under N₂. LCMS showed there were no starting material and main desired compound. The reaction mixture was added to water (100 mL). The resulting solution was extracted with EtOAc (30 mL×3). The organic layers were dried over anhydrous Na₂SO₄ and concentrated under reduced pressure to give a residue. The residue was purified by column chromatography (SiO₂, Petroleum ether/EtOAc=1:0 to 10:1). The product 4-fluoro-3-methyl-1H-pyrrolo[2,3-c]pyridine (2 g, 5.33 mmol, 51.77% yield, 40% purity) was obtained as a yellow solid. LCMS (ESI) m/z 137.1 [M-TMS+H]⁺

### 4-fluoro-3-methyl-1-(p-tolylsulfonyl)pyrrolo[2,3-c]pyridine

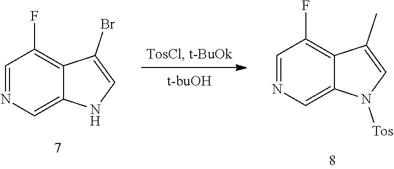

To a solution of 4-fluoro-3-methyl-1H-pyrrolo[2,3-c]pyridine (2 g, 13.32 mmol, 1 eq) in t-BuOH (15 mL) was added t-BuOK (3.74 g, 33.30 mmol, 2.5 eq) and TosCl (3.30 g, 17.32 mmol, 1.3 eq). The reaction was stirred at 30° C. for 12 h. LC-MS showed the starting material was consumed completely. The reaction mixture was added to water (100 mL). The resulting solution was extracted with EtOAc (30 mL×3). The organic layers were dried over anhydrous Na₂SO₄ and concentrated under reduced pressure to give a residue. The residue was purified by column chromatography (SiO₂, Petroleum ether/EtOAc=1:0 to 10:1). The product 4-fluoro-3-methyl-1-(p-tolylsulfonyl)pyrrolo[2,3-c]pyridine (750 mg, 2.46 mmol, 18.50% yield) was obtained as white solid. LCMS (ESI) m/z 305.1 [M+H]⁺

### 4-fluoro-3-methyl-1-(p-tolylsulfonyl)pyrrolo[2,3-c]pyridine-2-carboxylic acid

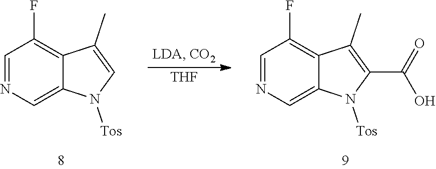

To a solution of 4-fluoro-3-methyl-1-(p-tolylsulfonyl)pyrrolo[2,3-c]pyridine (750 mg, 2.46 mmol, 1 eq) in THE (10 mL) was added LDA (2 M, 2.46 mL, 2 eq) under N₂ at −78° C. The mixture was stirred at −78° C. for 2 h. Then the mixture was stirred at −78° C. for 1 h under CO₂ (15 psi). LC-MS showed 3% of the starting material was remained. The reaction was quenched with saturated aqueous NH₄Cl (2 mL). The aqueous phase was adjusted with saturated aqueous Na₂CO₃ to pH=9. The mixture was washed with 20 mL×2 of EtOAc. The mixture was acidified with HCl (2 M) to pH=5. The mixture was filtered to give the product. The crude product was used directly for the next step without purification. The product 4-fluoro-3-methyl-1-(p-tolylsulfonyl)pyrrolo[2,3-c]pyridine-2-carboxylic acid (858.45 g, crude) was obtained as a yellow solid. LCMS (ESI) m/z 349.1 [M+H]⁺

### 4-fluoro-3-methyl-1H-pyrrolo[2,3-c]pyridine-2-carboxylic acid

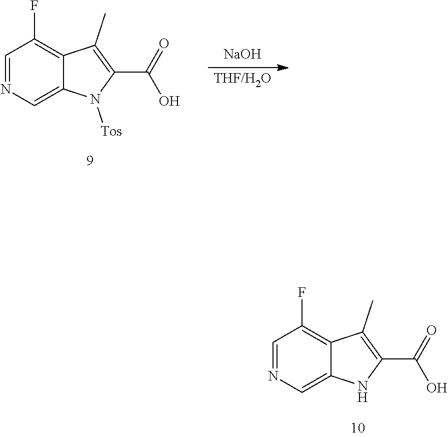

To a solution of 4-fluoro-3-methyl-1-(p-tolylsulfonyl)pyrrolo[2,3-c]pyridine-2-carboxylic acid (961.29 mg, 2.76 mmol, 1 eq) in THE (10 mL) was added NaOH (solution in water) (2 M, 10 mL, 7.25 eq). The mixture was stirred at 30° C. for 12 h. LCMS showed no starting material and desired mass was detected. The mixture was acidified with HCl (2 M) to pH=8 and concentrated under reduced pressure. The mixture was washed with EtOAc (20 mL) and acidified with HCl (2 M) to pH=5. The mixture was filtered and the filter cake was washed with 10 mL×3 of Petroleum ether, dried under reduced pressure to give product. The residue was diluted in CH₃CN (5 mL) and H₂O (20 mL), then lyophilized. The product 4-fluoro-3-methyl-1H-pyrrolo[2,3-c]pyridine-2-carboxylic acid (430 mg, 1.99 mmol, 72.23% yield, 90% purity) was obtained as a white solid. LCMS (ESI) m/z 195.0 [M+H]⁺

### N-(1,1-dimethylsilinan-4-yl)-4-fluoro-3-methyl-1H-pyrrolo[2,3-c]pyridine-2-carboxamide

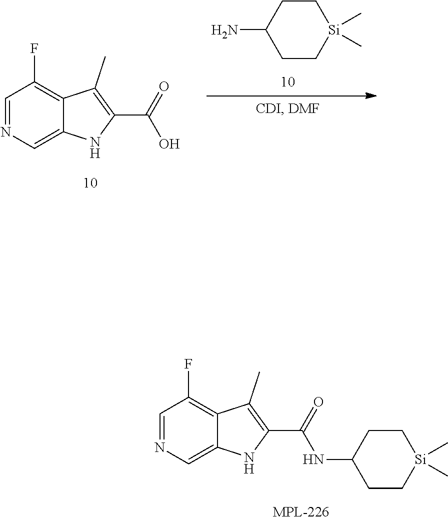

To a solution of 4-fluoro-3-methyl-1H-pyrrolo[2,3-c]pyridine-2-carboxylic acid (200 mg, 1.03 mmol, 1 eq) and CDI (200.43 mg, 1.24 mmol, 1.2 eq) in DMF (2.5 mL). The mixture was stirred at 30° C. for 3 h. 1,1-dimethylsilinan-4-amine (177.13 mg, 1.24 mmol, 1.2 eq) was added. The mixture was stirred at 30° C. for 1 h. LC-MS showed the starting material was consumed completely. The reaction mixture was added to water (20 mL), filtered and the filter cake was washed with 10 mL of water, dried in vacuo to give product. The crude product was purified by prep-HPLC (column: Phenomenex Synergi C18 150*30 mm*4 um; mobile phase: [water (0.05% HCl)-ACN]; B %: 28%-48%, 10 min), then lyophilized to give the product. The product N-(1,1-dimethylsilinan-4-yl)-4-fluoro-3-methyl-1H-pyrrolo[2,3-c]pyridine-2-carboxamide (74.8 mg, 216.26 umol, 20.99% yield, 92.359% purity) was obtained as a white solid.

LCMS (ESI) m/z 320.0 [M+H]⁺; ¹H NMR (500 MHz, DMSO-d6) δ=13.52 (br s, 1H), 8.99 (s, 1H), 8.58 (br d, J=7.8 Hz, 1H), 8.46 (d, J=4.4 Hz, 1H), 3.82-3.69 (m, 1H), 2.60 (s, 3H), 2.07-2.00 (m, 2H), 1.69-1.60 (m, 2H), 0.80 (br d, J=14.5 Hz, 2H), 0.62 (dt, J=4.8, 13.8 Hz, 2H), 0.08 (s, 3H), 0.04 (s, 3H).

### Example 35. MPL-229

### Synthesis of N-(1, 1-dimethylsilinan-4-yl)-4, 5-difluoro-6-methyl-1H-pyrrolo[2, 3-b]pyridine-2-carboxamide

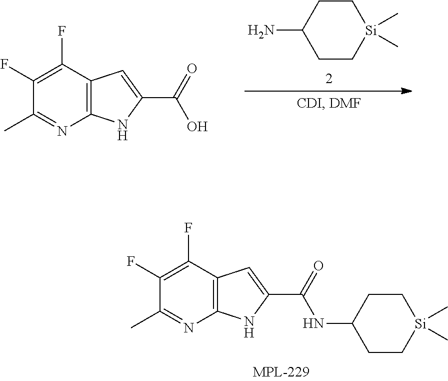

To a solution of 4, 5-difluoro-6-methyl-1H-pyrrolo[2, 3-b]pyridine-2-carboxylic acid (40 mg, 188.54 umol, 1 eq) in DMF (1.5 mL) was added CDI (33.63 mg, 207.40 umol, 1.1 eq). The mixture was stirred at 30° C. for 0.5 h. Then 1, 1-dimethylsilinan-4-amine (29.72 mg, 207.40 umol, 1.1 eq) was added. The mixture was stirred at 30° C. for 11.5 h. LCMS showed there were main desired compound and a little starting material. The reaction was added dropwise to H₂O (20 mL). There was much precipitation which was collected by filter. The cake was transferred in bottom flask. The crude product was purified by prep-TLC (SiO₂, Petroleum ether:EtOAc=5:1). Compound N-(1, 1-dimethylsilinan-4-yl)-4, 5-difluoro-6-methyl-1H-pyrrolo[2, 3-b]pyridine-2-carboxamide (30 mg, 88.39 umol, 46.88% yield, 99.423% purity) was obtained as a white solid.

LCMS (ESI), m/z 338.0[M+H]⁺; ¹H NMR (400 MHz, CHLOROFORM-d) 6=9.55 (br s, 1H), 6.80 (s, 1H), 6.04 (br d, J=8.2 Hz, 1H), 3.91 (br d, J=8.2 Hz, 1H), 2.63 (d, J=3.1 Hz, 3H), 2.18 (br d, J=10.2 Hz, 2H), 1.59-1.53 (m, 2H), 0.83-0.68 (m, 4H), 0.10 (s, 3H), 0.06 (s, 3H).

### Example 36. MPL-001

### Synthesis of 4-chloro-N-spiro[3.5]nonan-7-yl-1H-pyrrolo[2,3-b]pyridine-2-carboxamide

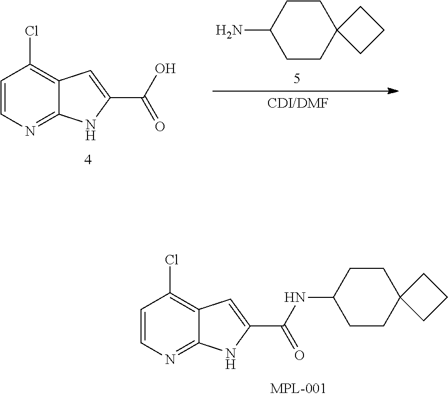 [SMILES: O=C(O)c1cc2c(Cl)ccnc2[nH]1 ; NC1CCC2(CCC2)CC1 ; O=C(NC1CCC2(CCC2)CC1)c1cc2c(Cl)ccnc2[nH]1]

To a solution of 4-chloro-1H-pyrrolo[2,3-b]pyridine-2-carboxylic acid (190 mg, 966.48 umol, 1 eq) in DMF (4 mL) was added CDI (188.06 mg, 1.16 mmol, 1.2 eq) and spiro[3.5]nonan-7-amine (188.40 mg, 1.35 mmol, 1.4 eq). The mixture was stirred at 30° C. for 12 hr. LCMS showed there were no starting material and main desired compound. There was much precipitation. The mixture was added dropwise to H₂O (20 mL). There was much precipitation which was collected by filter. The cake was washed with H₂O (10 mL). The solid was diluted with CH₃CN (1 mL) and H₂O (10 mL), then lyophilized. Compound 4-chloro-N-spiro[3.5]nonan-7-yl-1H-pyrrolo[2,3-b]pyridine-2-carboxamide (140 mg, 435.75 umol, 45.09% yield, 98.92% purity) was obtained as a white solid which was confirmed by LCMS and 1H NMR. LCMS (ESI) m/z 318.1 [M+H]⁺; ¹H NMR (500 MHz, DMSO-d₆) δ=12.48 (br s, 1H), 8.40-8.31 (m, 1H), 8.27 (d, J=5.0 Hz, 1H), 7.29-7.22 (m, 2H), 3.73 (br s, 1H), 1.88-1.81 (m, 2H), 1.80-1.73 (m, 4H), 1.70 (br d, J=7.8 Hz, 4H), 1.43-1.28 (m, 4H).

### Example 37. MPL-002

### Synthesis of 4-fluoro-N-spiro[3.5]nonan-7-yl-1H-pyrrolo[2,3-b]pyridine-2-carboxamide

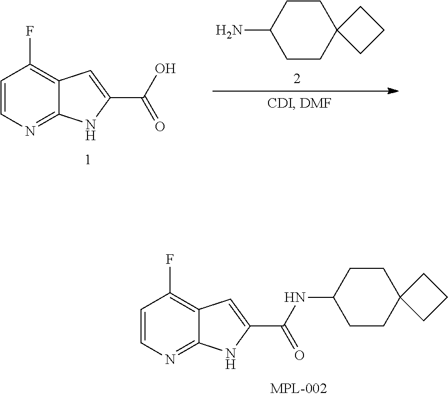 [SMILES: O=C(O)c1cc2c(F)ccnc2[nH]1 ; NC1CCC2(CCC2)CC1 ; O=C(NC1CCC2(CCC2)CC1)c1cc2c(F)ccnc2[nH]1]

To a solution of 4-fluoro-1H-pyrrolo[2,3-b]pyridine-2-carboxylic acid (200 mg, 1.11 mmol, 1 eq) in DMF (2 mL) was added CDI (234.04 mg, 1.44 mmol, 1.3 eq). The mixture was stirred at 30° C. for 0.5 h. spiro[3.5]nonan-7-amine (200.97 mg, 1.44 mmol, 1.3 eq) was added and the reaction mixture was stirred at 30° C. for 12 h. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. The residue was delivered without further purification. Compound 4-fluoro-N-spiro[3.5]nonan-7-yl-1H-pyrrolo[2,3-b]pyridine-2-carboxamide (210 mg, 674.76 umol, 60.77% yield, 96.83% purity) was obtained as a white solid which was confirmed by LCMS and ¹H NMR.

LCMS (ESI) m/z 302.1 [M+H]⁺; 1H NMR (400 MHz, DMSO-d₆) δ=12.43 (br s, 1H), 8.34-8.18 (m, 1H), 8.34-8.18 (m, 1H), 7.21 (s, 1H), 6.97 (dd, J=5.3, 10.1 Hz, 1H), 3.71 (br s, 1H), 1.84-1.63 (m, 10H), 1.40-1.26 (m, 1H), 1.40-1.26 (m, 1H), 1.40-1.23 (m, 2H).

### Example 38. MPL-003

### Synthesis of 4-chloro-N-(4,4-dimethylcyclohexyl)-1H-pyrrolo[2,3-c]pyridine-2-carboxamide

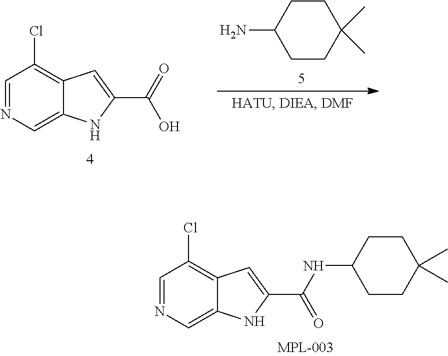

To a solution of 4-chloro-1H-pyrrolo[2,3-c]pyridine-2-carboxylic acid (800 mg, 4.07 mmol, 1 eq) in DMF (8 mL) was added CDI (989.77 mg, 6.10 mmol, 1.5 eq), the mixture was stirred at 30° C. for 2.5 h, then 4,4-dimethylcyclohexanamine (776.60 mg, 6.10 mmol, 1.5 eq) was added. The mixture was stirred at 30° C. for another 0.5 h. LC-MS showed 20% of the starting material 4 was remained and one main peak with desired mass was detected. The mixture was added to water (100 mL), filtered and the filter cake was washed with 20 mL×3 of Petroleum ether, dried under reduced pressure to give the product. The product 4-chloro-N-(4,4-dimethylcyclohexyl)-1H-pyrrolo[2,3-c]pyridine-2-carboxamide (692.4 mg, 2.24 mmol, 55.14% yield, 99.104% purity) was obtained as white solid.

LCMS (ESI) m/z 306.1 [M+H]⁺; ¹H NMR (400 MHz, DMSO-δ₆)=12.43 (br s, 1H), 8.72 (s, 1H), 8.59 (br d, J=7.6 Hz, 1H), 8.18 (s, 1H), 7.33 (s, 1H), 3.76 (br d, J=6.6 Hz, 1H), 1.67 (br d, J=10.3 Hz, 2H), 1.60-1.50 (m, 2H), 1.45-1.37 (m, 2H), 1.34-1.24 (m, 2H), 0.94 (br d, J=9.0 Hz, 6H).

### Example 39. MPL-006

### Synthesis of 4-chloro-N-[(1S,2S,3S,5R)-2,6,6-trimethylnorpinan-3-yl]-1H-pyrrolo[2,3-c]Pyridine-2-carboxamide

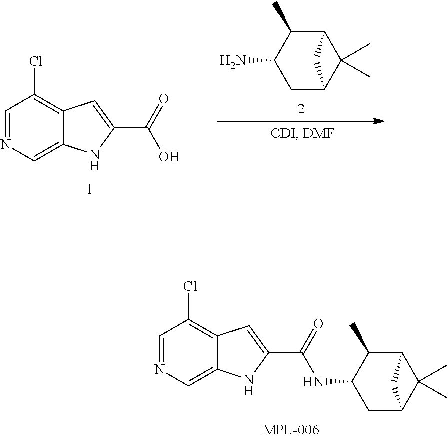 [SMILES: O=C(O)c1cc2c(Cl)cncc2[nH]1 ; C[C@@H]1[C@@H](N)C[C@H]2C[C@@H]1C2(C)C ; C[C@@H]1[C@@H](NC(=O)c2cc3c(Cl)cncc3[nH]2)C[C@H]2C[C@@H]1C2(C)C]

To a solution of 4-chloro-1H-pyrrolo[2,3-c]pyridine-2-carboxylic acid (250 mg, 1.27 mmol, 1 eq) in DMF (3 mL) was added CDI (268.06 mg, 1.65 mmol, 1.3 eq). The mixture was stirred at 30° C. for 0.5 h. Then (1S,2S,3S,5R)-2,6,6-trimethylnorpinan-3-amine (292.35 mg, 1.91 mmol, 1.5 eq) was added. The mixture was stirred at 30° C. for 11.5 h. LCMS showed there was no starting material. The reaction was added dropwise to H₂O (20 mL). There was much precipitation which was collected by filter. The cake was diluted with EtOAc (30 mL), dried with anhydrous MgSO₄, filtered. The filtrate was concentrated in vacuo. The residue was purified by column chromatography (SiO₂, Petroleum ether:EtOAc=1:1). Compound 4-chloro-N-[(1S,2S,3S,5R)-2,6,6-trimethylnorpinan-3-yl]-1H-pyrrolo[2,3-c]pyridine-2-carboxamide (259 mg, 775.07 umol, 60.95% yield, 99.305% purity) was obtained as a white solid.

LCMS (ESI), m/z 331.15[M+H]⁺; ¹H NMR (400 MHz, DMSO-d₆) δ=12.46 (br s, 1H), 8.74 (s, 1H), 8.71 (br d, J=8.6 Hz, 1H), 8.19 (s, 1H), 7.39 (s, 1H), 4.46-4.35 (m, 1H), 2.48-2.35 (m, 2H), 2.10 (br t, J=7.2 Hz, 1H), 2.01-1.92 (m, 1H), 1.83 (br t, J=5.1 Hz, 1H), 1.72 (br dd, J=6.4, 11.7 Hz, 1H), 1.27-1.20 (m, 4H), 1.11-1.05 (m, 6H).

### Example 40. MPL-007

### Synthesis of 4-bromo-N-(4,4-dimethylcyclohexyl)-1H-pyrrolo[2,3-b]pyridine-2-carboxamide

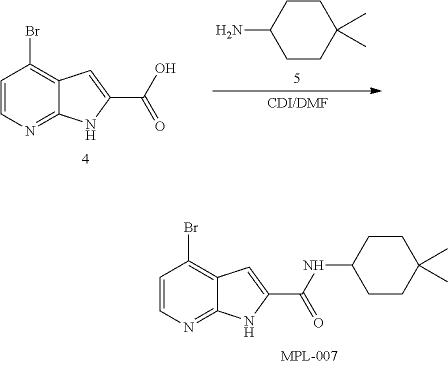

To a solution of 4-bromo-1H-pyrrolo[2,3-b]pyridine-2-carboxylic acid (2.00 g, 8.30 mmol, 1 eq) and CDI (2.02 g, 12.45 mmol, 1.5 eq) in DMF (20 mL), the mixture was stirred at 25° C. for 30 min, then 4,4-dimethylcyclohexanamine (1.58 g, 12.45 mmol, 1.5 eq) was added, the mixture was stirred at 25° C. for 0.5 h under N₂. LC-MS showed the starting material 4 was consumed completely and one main peak with desired mass was detected. The mixture was added to a solution of LiCl (300 ml, 3%) and filtered. The filter cake was washed with 50 mL of water, dried under reduced pressure to give product. The product 4-bromo-N-(4,4-dimethylcyclohexyl)-1H-pyrrolo[2,3-b]pyridine-2-carboxamide (2.24 g, 6.40 mmol, 77.14% yield, 100% purity) was obtained as white solid.

LCMS (ESI) m/z 352.1 [M+H]⁺; ¹H NMR (400 MHz, DMSO-δ₆) δ=12.48 (br s, 1H), 8.42 (br d, J=7.6 Hz, 1H), 8.18 (br d, J=4.9 Hz, 1H), 7.40 (br d, J=4.9 Hz, 1H), 7.20 (s, 1H), 3.73 (br d, J=7.8 Hz, 1H), 1.67 (br d, J=10.7 Hz, 2H), 1.59-1.50 (m, 2H), 1.41 (br d, J=12.5 Hz, 2H), 1.33-1.24 (m, 2H), 0.94 (br d, J=11.0 Hz, 6H).

### Example 41. MPL-008

### Synthesis of 4-cyano-N-(4,4-dimethylcyclohexyl)-1H-pyrrolo[2,3-b]pyridine-2-carboxamide

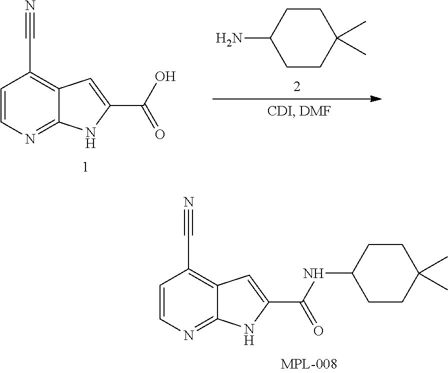

To a solution of 4-cyano-1H-pyrrolo[2,3-b]pyridine-2-carboxylic acid (500 mg, 2.67 mmol, 1 eq) in DMF (8 mL) was added CDI (563.15 mg, 3.47 mmol, 1.3 eq). The mixture was stirred at 30° C. for 0.5 h. 4,4-dimethylcyclohexanamine (441.87 mg, 3.47 mmol, 1.3 eq) was added and the reaction mixture was stirred at 30° C. for 12 h. LCMS showed there were no starting material and main desired compound. The reaction was added dropwise to H₂O (50 mL). There was much precipitation which was collected by filter. The cake was diluted in CH₃CN (5 mL) and H₂O (20 mL), then lyophilized. The residue was delivered without further purification. Compound 4-cyano-N-(4,4-dimethylcyclohexyl)-1H-pyrrolo[2,3-b]pyridine-2-carboxamide (520 mg, 1.74 mmol, 65.29% yield, 99.41% purity) was obtained as a white solid which was confirmed by LCMS and ¹H NMR.

LCMS (ESI) m/z 297.1 [M+H]⁺; 1H NMR (500 MHz, DMSO-d₆) δ=12.87 (br s, 1H), 8.54-8.50 (m, 1H), 8.54-8.50 (m, 1H), 7.65 (d, J=4.9 Hz, 1H), 7.43 (s, 1H), 3.81-3.71 (m, 1H), 1.69 (br dd, J=3.6, 13.0 Hz, 2H), 1.61-1.50 (m, 2H), 1.43 (br d, J=12.5 Hz, 2H), 1.34-1.26 (m, 2H), 0.95 (d, J=11.1 Hz, 6H).

### Example 42. MPL-009

### Synthesis of 4-methoxy-1H-pyrrolo[2,3-b]pyridine-2-carbonyl chloride

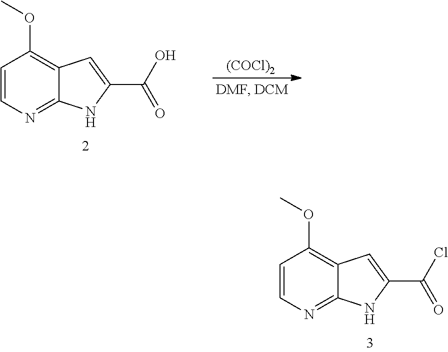

To a solution of 4-methoxy-1H-pyrrolo[2,3-b]pyridine-2-carboxylic acid (300 mg, 1.56 mmol, 1 eq) in DCM (10 mL) was added DMF (5.71 mg, 78.06 umol, 6.01 uL, 0.05 eq) and (COCl)₂ (2.90 g, 22.85 mmol, 2 mL, 14.64 eq). The mixture was stirred at 25° C. for 1 hr. LCMS showed the starting material 2 was consumed and desire product formed. The mixture was directly concentrated under reduce pressure to give a residue. The residue was directly used in next step without any purification. Compound 4-methoxy-1H-pyrrolo[2,3-b]pyridine-2-carbonyl chloride (300 mg, 1.35 mmol, 86.68% yield, 95% purity) was obtained as a white solid. LCMS (ESI) m/z 207.1 [M+H]⁺

### Synthesis of N-(4,4-dimethylcyclohexyl)-4-methoxy-1H-pyrrolo[2,3-b]pyridine-2-carboxamide

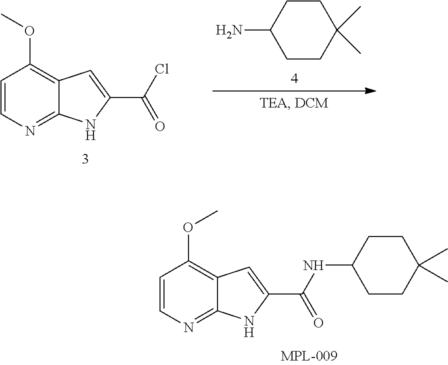

To a solution of 4-methoxy-1H-pyrrolo[2,3-b]pyridine-2-carbonyl chloride (300 mg, 1.42 mmol, 1 eq) in DCM (8 mL) was added TEA (288.27 mg, 2.85 mmol, 396.52 uL, 2 eq) and 4,4-dimethylcyclohexanamine (181.22 mg, 1.42 mmol, 1 eq). The mixture was stirred at 25° C. for 1 hr. LCMS showed the starting material 3 was consumed and desire product formed. The mixture was directly concentrated under reduce pressure to give a residue. The residue was purified by column chromatography (SiO₂, Petroleum ether:EtOAc=10:1 to 0:1). Compound N-(4,4-dimethylcyclohexyl)-4-methoxy-1H-pyrrolo[2,3-b]pyridine-2-carboxamide (85 mg, 282.03 umol, 19.80% yield, 100% purity) was obtained as a white solid.

LCMS (ESI) m/z 302.2 [M+H]⁺; ¹H NMR (400 MHz, DMSO-d6) δ=11.97 (br s, 1H), 8.19 (d, J=5.5 Hz, 1H), 8.13 (br d, J=7.9 Hz, 1H), 7.18 (d, J=2.0 Hz, 1H), 6.68 (d, J=5.6 Hz, 1H), 3.97 (s, 3H), 3.78-3.66 (m, 1H), 1.73-1.63 (m, 2H), 1.59-1.37 (m, 4H), 1.28 (dt, J=3.7, 13.2 Hz, 2H), 0.94 (d, J=7.8 Hz, 6H).

### Example 43. MPL-012

### Synthesis of 4-bromo-N-[(1S,2S,3S,5R)-2,6,6-trimethylnorpinan-3-yl]-1H-pyrrolo[2,3-b]pyridine-2-carboxamide

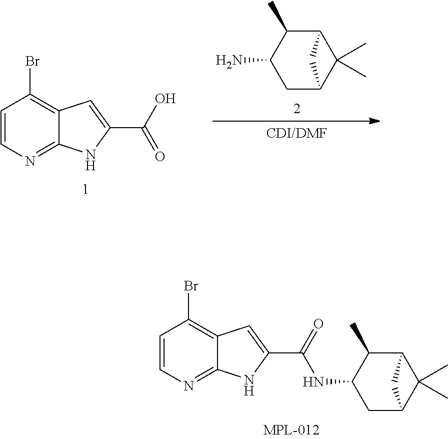 [SMILES: O=C(O)c1cc2c(Br)ccnc2[nH]1 ; C[C@@H]1[C@@H](N)C[C@H]2C[C@@H]1C2(C)C ; C[C@@H]1[C@@H](NC(=O)c2cc3c(Br)ccnc3[nH]2)C[C@H]2C[C@@H]1C2(C)C]

To a solution of 4-bromo-1H-pyrrolo[2,3-b]pyridine-2-carboxylic acid (2.00 g, 8.30 mmol, 1 eq) and CDI (2.02 g, 12.45 mmol, 1.5 eq) in DMF (20 mL) the mixture was stirred at 25° C. for 30 min, then (1S,2S,3S,5R)-2,6,6-trimethylnorpinan-3-amine (1.53 g, 9.96 mmol, 1.2 eq) was added, the mixture was stirred at 25° C. for 0.5 h under N₂. LC-MS showed the starting material 1 was consumed completely and one main peak with desired mass was detected. The mixture was added to a solution of LiCl (300 ml, 3%) and filtered, the filter cake was washed with 50 mL of water, dried under reduced pressure to give the product. The product 4-bromo-N-[(1S,2S,3S,5R)-2,6,6-trimethylnorpinan-3-yl]-1H-pyrrolo[2,3-b]pyridine-2-carboxamide (1.85 g, 4.74 mmol, 57.09% yield, 96.464% purity) was obtained as white solid.

LCMS (ESI) m/z 377.9 [M+H]⁺; ¹H NMR (400 MHz, DMSO-δ₆) 6=12.52 (br s, 1H), 8.55 (br d, J=8.4 Hz, 1H), 8.19 (br d, J=5.0 Hz, 1H), 7.41 (br d, J=5.0 Hz, 1H), 7.24 (s, 1H), 4.38 (br s, 1H), 2.47-2.39 (m, 2H), 2.08 (br t, J=7.0 Hz, 1H), 1.95 (br s, 1H), 1.82 (br d, J=5.2 Hz, 1H), 1.71 (br dd, J=5.4, 12.7 Hz, 1H), 1.25-1.18 (m, 4H), 1.10-1.04 (m, 6H).

### Example 44. MPL-014

### Synthesis of 4-methoxy-1H-pyrrolo[2,3-b]pyridine-2-carbonyl chloride

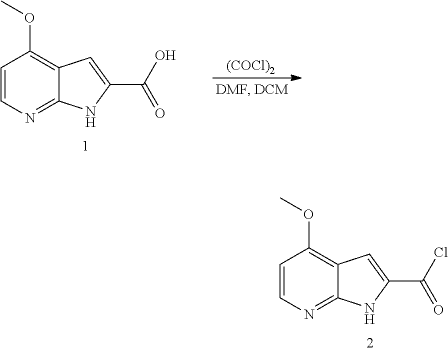

To a solution of 4-methoxy-1H-pyrrolo[2,3-b]pyridine-2-carboxylic acid (300 mg, 1.56 mmol, 1 eq) in DCM (10 mL) was added DMF (5.71 mg, 78.06 umol, 6.01 uL, 0.05 eq) and (COCl)₂ (2.90 g, 22.85 mmol, 2 mL, 14.64 eq). The mixture was stirred at 25° C. for 1 hr. LCMS showed the starting material 1 was consumed and desire product formed. The mixture was directly concentrated under reduce pressure to give a residue. The residue was directly used in next step without any purification. Compound 4-methoxy-1H-pyrrolo[2,3-b]pyridine-2-carbonyl chloride (300 mg, 1.35 mmol, 86.68% yield, 95% purity) was obtained as a white solid.

### Synthesis of 4-methoxy-N-[(1S,2S,3S,5R)-2,6,6-trimethylnorpinan-3-yl]-1H-pyrrolo[2,3-b]pyridine-2-carboxamide

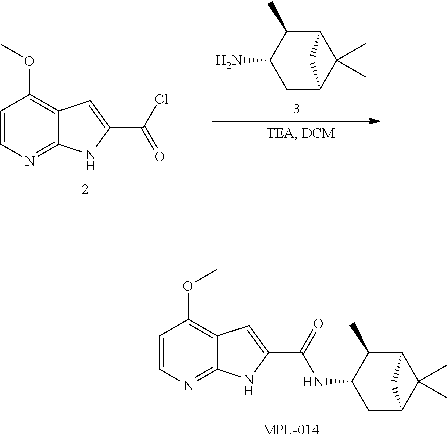 [SMILES: COc1ccnc2[nH]c(C(=O)Cl)cc12 ; C[C@@H]1[C@@H](N)C[C@H]2C[C@@H]1C2(C)C ; COc1ccnc2[nH]c(C(=O)N[C@H]3C[C@H]4C[C@@H]([C@@H]3C)C4(C)C)cc12]

To a solution of 4-methoxy-1H-pyrrolo[2,3-b]pyridine-2-carbonyl chloride (300 mg, 1.42 mmol, 1 eq) in DCM (8 mL) was added TEA (288.27 mg, 2.85 mmol, 396.52 uL, 2 eq) and (1S,2S,3S,5R)-2,6,6-trimethylnorpinan-3-amine (174.65 mg, 1.14 mmol, 0.8 eq) was added. The mixture was stirred at 25° C. for 2 hrs. LCMS showed the starting material 2 was consumed and desire product formed. The mixture was directly concentrated under reduce pressure to give a residue. The residue was purified by column chromatography (SiO₂, Petroleum ether:EtOAc=10:1 to 0:1). Compound 4-methoxy-N-[(1S,2S,3S,5R)-2,6,6-trimethylnorpinan-3-yl]-1H-pyrrolo[2,3-b]pyridine-2-carboxamide (100 mg, 305.42 umol, 21.44% yield, 100% purity) was obtained as a white solid. LCMS (ESI) m/z 328.2 [M+H]⁺

### Example 45. MPL-018

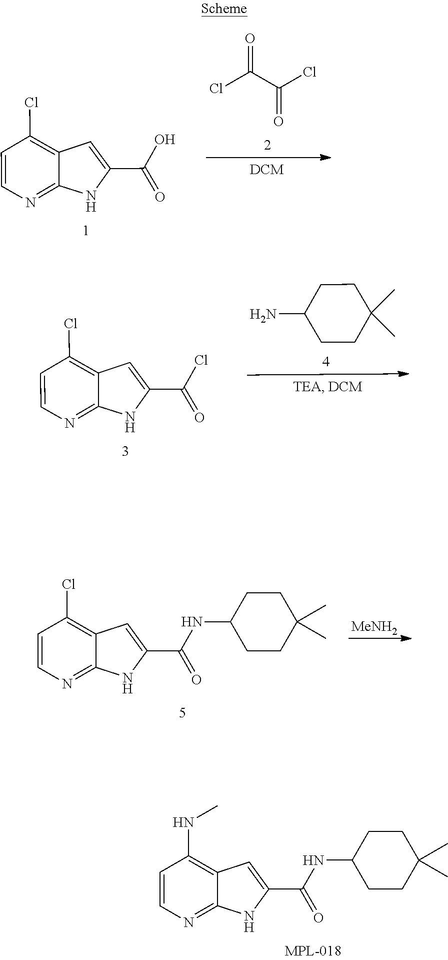

### Ethyl 2-methyl-4H-pyrrolo[2,3-d]thiazole-5-carboxylate

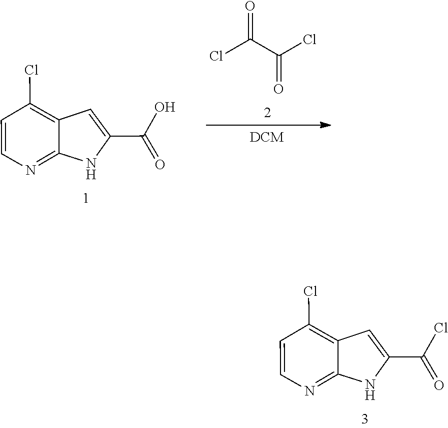 [SMILES: O=C(O)c1cc2c(Cl)ccnc2[nH]1 ; O=C(Cl)C(=O)Cl ; O=C(Cl)c1cc2c(Cl)ccnc2[nH]1]

To a solution of 4-chloro-1H-pyrrolo[2,3-b]pyridine-2-carboxylic acid (100.00 mg, 508.67 umol, 1 eq) in DCM (5 mL) was added oxalyl dichloride (645.64 mg, 5.09 mmol, 445.27 uL, 10 eq) and DMF (1.12 mg, 15.26 umol, 1.17 uL, 0.03 eq) under N₂, the mixture was stirred at 80° C. for 12 hrs. LC-MS showed the starting material 1 was consumed completely and one main peak with desired mass was detected. The mixture was concentrated under reduced pressure to give a residue. The crude product 4-chloro-1H-pyrrolo[2,3-b]pyridine-2-carbonyl chloride (109 mg, 506.89 umol, 99.65% yield) was obtained as yellow solid and used directly for the next step without purification.

### Synthesis of 4-chloro-N-(4,4-dimethylcyclohexyl)-1H-pyrrolo[2,3-b]pyridine-2-carboxamide

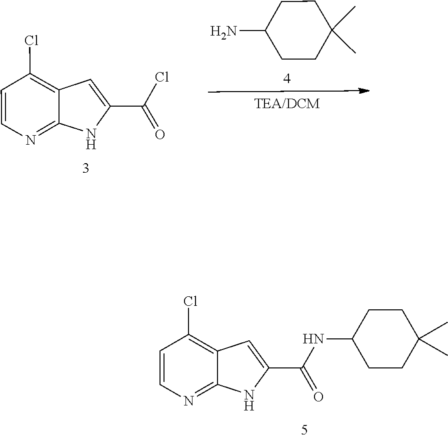

To a solution of 4-chloro-1H-pyrrolo[2,3-b]pyridine-2-carbonyl chloride (109 mg, 506.89 umol, 1 eq) in DCM (3 mL) was added 4,4-dimethylcyclohexanamine (161.23 mg, 1.27 mmol, 2.5 eq) and TEA (128.23 mg, 1.27 mmol, 176.38 uL, 2.5 eq), the mixture was stirred at 25° C. for 0.5 hr under N₂. TLC and LC-MS showed the starting material 3 was consumed completely and one main peak with desired mass was detected. The mixture was diluted with DCM:MeOH (30 mL) and washed with brine (30 mL), then extracted with DCM:MeOH (30 mL×3). The organic layers were dried over anhydrous Na₂SO₄ and concentrated under reduced pressure to give a residue. The residue was purified by column chromatography (SiO₂, DCM:MeOH=1:0 to 100:1). The product 4-chloro-N-(4,4-dimethylcyclohexyl)-1H-pyrrolo[2,3-b]pyridine-2-carboxamide (110.2 mg, 353.97 umol, 69.83% yield, 98.226% purity) was obtained as white solid. LCMS (ESI) m/z 306.1 [M+H]⁺

### Synthesis of N-(4,4-dimethylcyclohexyl)-4-(methylamino)-1H-pyrrolo[2,3-b]pyridine-2-carboxamide

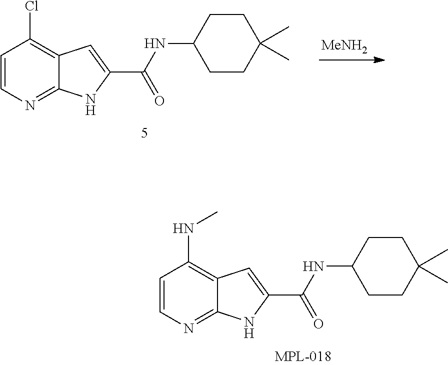 [SMILES: "MeNH2" ; CC1(C)CCC(NC(=O)c2cc3c(Cl)ccnc3[nH]2)CC1 ; CNc1ccnc2[nH]c(C(=O)NC3CCC(C)(C)CC3)cc12]

To a solution of 4-chloro-N-(4,4-dimethylcyclohexyl)-1H-pyrrolo[2,3-b]pyridine-2-carboxamide (100 mg, 327.01 umol, 1 eq) in methanamine (33.85 mg, 327.01 umol, 5 mL, 1 eq), the mixture was stirred at 120° C. for 24 hrs in a 30 mL of autoclave. TLC and LC-MS showed the starting material 1 was consumed completely and one main peak with desired mass was detected. The reaction mixture was concentrated under reduced pressure to give a residue. The residue was purified by column chromatography (SiO₂, DCM:MeOH=1:0 to 25:1) and prep. HPLC (column: Phenomenex Synergi C18 150*30 mm*4 um; mobile phase: [water (0.225% FA)-ACN]; B %: 14%-34%, 11 min). The product N-(4,4-dimethylcyclohexyl)-4-(methylamino)-1H-pyrrolo[2,3-b]pyridine-2-carboxamide (42.1 mg, 118.74 umol, 36.31% yield, 97.702% purity, FA) was obtained as white solid.

LCMS (ESI) m/z 301.2 [M+H]⁺; ¹H NMR (400 MHz, DMSO-δ₆) δ=7.99 (br d, J=7.1 Hz, 1H), 7.91 (d, J=5.7 Hz, 1H), 7.19-7.16 (m, 1H), 7.11 (br s, 1H), 6.14 (br d, J=5.5 Hz, 1H), 3.77-3.64 (m, 1H), 2.89 (br d, J=4.6 Hz, 3H), 1.74-1.63 (m, 2H), 1.54-1.38 (m, 4H), 1.32-1.21 (m, 2H), 0.93 (d, J=7.3 Hz, 6H).

### Example 46. MPL-023

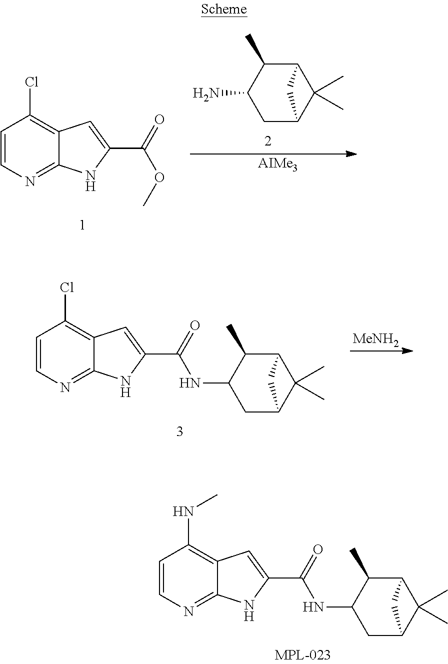 [SMILES: "MeNH2" ; COC(=O)c1cc2c(Cl)ccnc2[nH]1 ; C[C@@H]1[C@@H](N)C[C@H]2C[C@@H]1C2(C)C ; C[C@@H]1C(NC(=O)c2cc3c(Cl)ccnc3[nH]2)C[C@H]2C[C@@H]1C2(C)C ; CNc1ccnc2[nH]c(C(=O)NC3C[C@H]4C[C@@H]([C@@H]3C)C4(C)C)cc12 ; "AIMe3"]

### Synthesis of 4-chloro-N-[(1S,2S,3S,5R)-2,6,6-trimethylnorpinan-3-yl]-1H-pyrrolo[2,3-b]pyridine-2-carboxamide

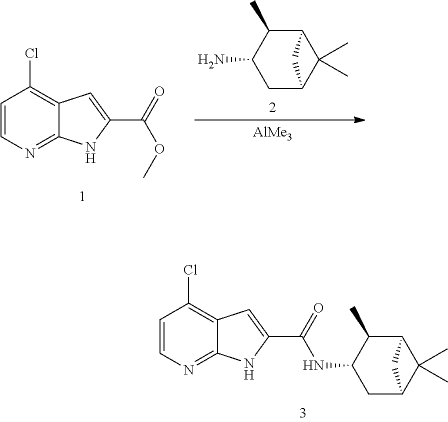 [SMILES: COC(=O)c1cc2c(Cl)ccnc2[nH]1 ; C[C@@H]1[C@@H](N)C[C@H]2C[C@@H]1C2(C)C ; C[C@@H]1[C@@H](NC(=O)c2cc3c(Cl)ccnc3[nH]2)C[C@H]2C[C@@H]1C2(C)C ; "AlMe3"]

To a solution of methyl 4-chloro-1H-pyrrolo[2,3-b]pyridine-2-carboxylate (150 mg, 712.19 umol, 1 eq) in DCE (5 mL) was added (1S,2S,3S,5R)-2,6,6-trimethylnorpinan-3-amine (327.46 mg, 2.14 mmol, 3 eq) and trimethylalumane (2 M, 712.19 uL, 2 eq). The mixture was stirred at 50° C. for 36 hrs. TLC and LC-MS showed the starting material 1 was consumed completely and one main peak with desired mass was detected. The mixture was diluted with water (30 mL) and extracted with DCM:MeOH (30 mL×3). The organic layers were dried over anhydrous Na₂SO₄ and concentrated under reduced pressure to give a residue. The residue was purified by column chromatography (SiO₂, DCM:MeOH=1:0 to 200:1). The product 4-chloro-N-[(1S,2S,3S,5R)-2,6,6-trimethylnorpinan-3-yl]-1H-pyrrolo[2,3-b]pyridine-2-carboxamide (149 mg, 359.21 umol, 50.44% yield, 80% purity) was obtained as white solid.

### Synthesis of 4-(methylamino)-N-[(1S,2S,3S,5R)-2,6,6-trimethylnorpinan-3-yl]-1H-pyrrolo[2,3-b]pyridine-2-carboxamide

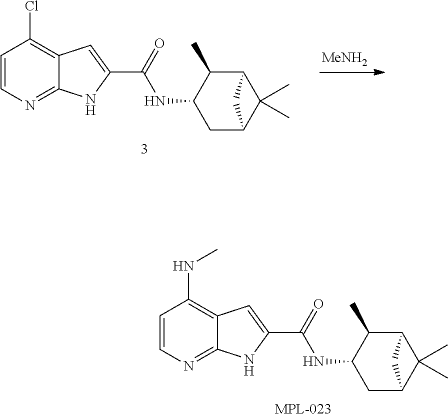 [SMILES: "MeNH2" ; C[C@@H]1[C@@H](NC(=O)c2cc3c(Cl)ccnc3[nH]2)C[C@H]2C[C@@H]1C2(C)C ; CNc1ccnc2[nH]c(C(=O)N[C@H]3C[C@H]4C[C@@H]([C@@H]3C)C4(C)C)cc12]

To a solution of 4-chloro-N-[(1S,2S,3S,5R)-2,6,6-trimethylnorpinan-3-yl]-1H-pyrrolo[2,3-b]pyridine-2-carboxamide (100 mg, 301.35 umol, 1 eq) in methanamine (31.20 mg, 301.35 umol, 5 mL, 1 eq), the mixture was stirred at 120° C. for 12 hr in a 30 mL of autoclave. TLC and LC-MS showed the starting material 3 was consumed completely and one main peak with desired mass was detected. The reaction mixture was concentrated under reduced pressure to give a residue. The residue was purified by column chromatography (SiO₂, DCM:MeOH=1:0 to 50:1) and prep. HPLC (column: Phenomenex Synergi C18 150*30 mm*4 um; mobile phase: [water (0.225% FA)-ACN]; B %: 17%-37%, 11 min). The product 4-(methylamino)-N-[(1S,2S,3S,5R)-2,6,6-trimethylnorpinan-3-yl]-1H-pyrrolo[2,3-b]pyridine-2-carboxamide (41.5 mg, 109.15 umol, 36.22% yield, 97.963% purity, FA) was obtained as white solid.

LCMS (ESI) m/z 327.2 [M+H]⁺; ¹H NMR (400 MHz, DMSO-δ₆) δ=8.17 (br d, J=8.4 Hz, 1H), 7.92 (d, J=5.7 Hz, 1H), 7.33-7.25 (m, 1H), 7.22 (s, 1H), 6.18 (d, J=5.7 Hz, 1H), 4.37-4.29 (m, 1H), 2.91 (d, J=4.6 Hz, 3H), 2.46-2.36 (m, 2H), 2.08-2.00 (m, 1H), 1.94 (br s, 1H), 1.82 (br t, J=5.3 Hz, 1H), 1.70-1.62 (m, 1H), 1.23 (s, 3H), 1.17-1.12 (m, 1H), 1.06 (t, J=3.6 Hz, 6H).

### Example 47. MPL-027

### Synthesis of 4-fluoro-1H-pyrrolo[2,3-b]pyridine-2-carbonyl chloride

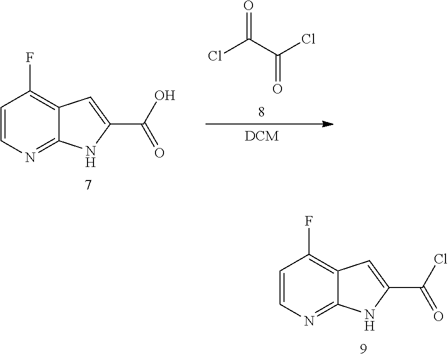 [SMILES: O=C(O)c1cc2c(F)ccnc2[nH]1 ; O=C(Cl)C(=O)Cl ; O=C(Cl)c1cc2c(F)ccnc2[nH]1]

To a solution of 4-fluoro-1H-pyrrolo[2,3-b]pyridine-2-carboxylic acid (100 mg, 555.14 umol, 1 eq) in DCM (5 mL) was added oxalyl dichloride (1.41 g, 11.10 mmol, 971.88 uL, 20 eq) and DMF (1.22 mg, 16.65 umol, 1.28 uL, 0.03 eq) under N2, the mixture was stirred for 1.5 hr at 25° C. under N₂. TLC showed the starting material 7 was consumed completely and one main spot was detected. The mixture was concentrated under reduced pressure to give a residue. After concentration, the crude product as a yellow solid was used directly for the next step without purification. The product 4-fluoro-1H-pyrrolo[2,3-b]pyridine-2-carbonyl chloride (110.24 mg, 555.14 umol, 100.00% yield) as yellow solid was obtained.

### Synthesis of N-cyclooctyl-4-fluoro-1H-pyrrolo[2,3-b]pyridine-2-carboxamide

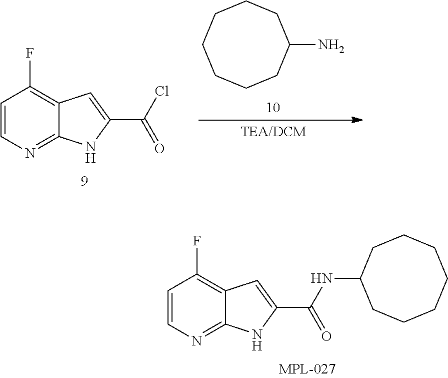 [SMILES: O=C(Cl)c1cc2c(F)ccnc2[nH]1 ; NC1CCCCCCC1 ; O=C(NC1CCCCCCC1)c1cc2c(F)ccnc2[nH]1]

To a solution of 4-fluoro-1H-pyrrolo[2,3-b]pyridine-2-carbonyl chloride (110 mg, 553.93 umol, 1 eq) in DCM (3 mL) was added cyclooctanamine (105.71 mg, 830.89 umol, 1.5 eq) and TEA (112.10 mg, 1.11 mmol, 154.20 uL, 2 eq) under N₂, the mixture was stirred at 25° C. for 12 hrs under N₂. TLC and LC-MS showed the starting material 1 was consumed completely and one main peak with desired mass was detected. The reaction mixture was diluted with DCM (20 mL) and washed with HCl (1 M, 20 mL) and then extracted with DCM (20 mL×3). The combined organic layers were dried over anhydrous Na₂SO₄, filtered and concentrated under reduced pressure to give a residue. The residue was purified by column chromatography (SiO₂, DCM:MeOH=1:0 to 10:1). The product N-cyclooctyl-4-fluoro-1H-pyrrolo[2,3-b]pyridine-2-carboxamide (18.5 mg, 62.33 umol, 11.25% yield, 97.488% purity) was obtained as white solid.

LCMS (ESI) m/z 290.2 [M+H]⁺; ¹H NMR (400 MHz, DMSO-δ₆) δ=12.44 (br s, 1H), 8.32 (dt, J=3.0, 5.3 Hz, 2H), 7.25 (d, J=2.0 Hz, 1H), 7.01 (dd, J=5.4, 10.3 Hz, 1H), 4.10-3.96 (m, 1H), 1.81-1.67 (m, 6H), 1.59-1.46 (m, 8H).

### Example 48. MPL-028

### Synthesis of 4-fluoro-N-(4-methylcyclohexyl)-1H-pyrrolo[2,3-b]pyridine-2-carboxamide

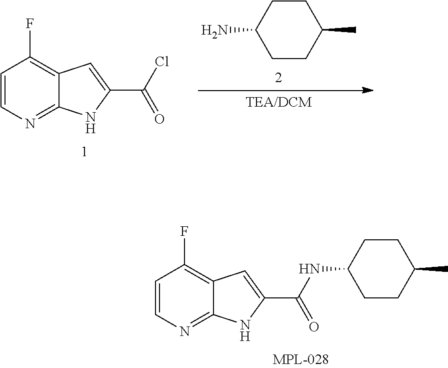

To a solution of 4-fluoro-1H-pyrrolo[2,3-b]pyridine-2-carbonyl chloride (110 mg, 553.93 umol, 1 eq) in DCM (3 mL) was added 4-methylcyclohexanamine (94.06 mg, 830.90 umol, 1.5 eq) and TEA (112.10 mg, 1.11 mmol, 154.20 uL, 2 eq) under N2, the mixture was stirred at 25° C. for 3 hrs under N₂. TLC and LC-MS showed the starting material 1 was consumed completely and one main peak with desired mass was detected. The reaction mixture was diluted with DCM (20 mL) and washed with HCl (1 M, 20 mL) and then extracted with DCM (20 mL×3). The combined organic layers were dried over anhydrous Na₂SO₄, filtered and concentrated under reduced pressure to give a residue. The residue was purified by column chromatography (SiO₂, DCM:MeOH=1:0 to 10:1). The product 1 4-fluoro-N-(4-methylcyclohexyl)-1H-pyrrolo[2,3-b]pyridine-2-carboxamide (24.3 mg, 84.80 umol, 15.31% yield, 96.08% purity) was obtained as white solid.

LCMS (ESI) m/z 275.1 [M+H]⁺; ¹H NMR (400 MHz, DMSO-δ₆) δ=12.45 (br s, 1H), 8.35-8.27 (m, 2H), 7.23 (s, 1H), 7.00 (dd, J=5.4, 10.3 Hz, 1H), 3.79-3.66 (m, 1H), 1.86 (br d, J=9.7 Hz, 2H), 1.71 (br d, J=12.3 Hz, 2H), 1.41-1.28 (m, 3H), 1.10-0.97 (m, 2H), 0.89 (d, J=6.4 Hz, 3H).

### Example 49. MPL-033

### Synthesis of 4-chloro-1H-pyrrolo[2,3-b]pyridine-2-carbonyl chloride

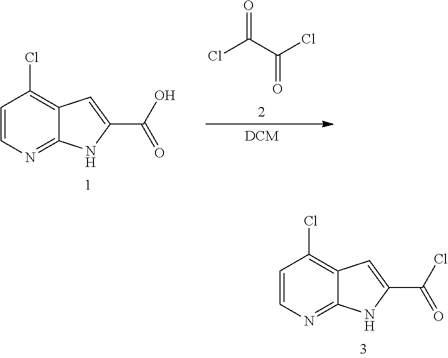 [SMILES: O=C(O)c1cc2c(Cl)ccnc2[nH]1 ; O=C(Cl)C(=O)Cl ; O=C(Cl)c1cc2c(Cl)ccnc2[nH]1]

To a solution of 4-chloro-1H-pyrrolo[2,3-b]pyridine-2-carboxylic acid (100.00 mg, 508.67 umol, 1 eq) in DCM (5 mL) was added oxalyl dichloride (645.64 mg, 5.09 mmol, 445.27 uL, 10 eq) and DMF (1.12 mg, 15.26 umol, 1.17 uL, 0.03 eq) under N2, the mixture was stirred at 80° C. for 1.5 hrs. LC-MS showed the starting material 1 was consumed completely and one main peak with desired mass was detected. The mixture was concentrated under reduced pressure to give a residue. The crude product 4-chloro-1H-pyrrolo[2,3-b]pyridine-2-carbonyl chloride (109 mg, 506.89 umol, 99.65% yield) was obtained as yellow solid and used directly for the next step without purification.

### Synthesis of 4-chloro-N-cyclooctyl-1H-pyrrolo[2,3-b]pyridine-2-carboxamide

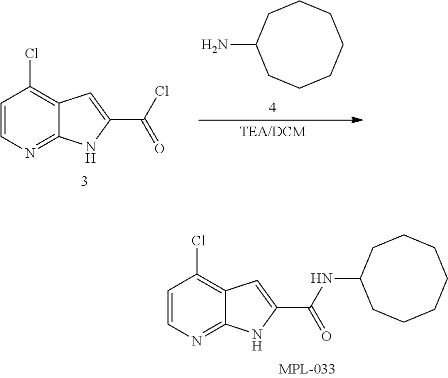 [SMILES: O=C(Cl)c1cc2c(Cl)ccnc2[nH]1 ; NC1CCCCCCC1 ; O=C(NC1CCCCCCC1)c1cc2c(Cl)ccnc2[nH]1]

To a solution of 4-chloro-1H-pyrrolo[2,3-b]pyridine-2-carbonyl chloride (109 mg, 506.89 umol, 1 eq) and cyclooctanamine (128.98 mg, 1.01 mmol, 2 eq) in DCM (3 mL) was added TEA (102.58 mg, 1.01 mmol, 141.11 uL, 2 eq), the mixture was stirred at 25° C. for 0.5 hr under N₂. TLC and LC-MS showed the starting material 3 was consumed completely and one main peak with desired mass was detected. The reaction mixture was diluted with solvent of DCM:MeOH=10:1 (40 mL) and washed with brine (20 mL×2), then the organic layers were dried over anhydrous Na₂SO₄, filtered and concentrated under reduced pressure to give a residue. The residue was purified by column chromatography (SiO₂, DCM:MeOH=1:0 to 100:1). The product 4-chloro-N-cyclooctyl-1H-pyrrolo[2,3-b]pyridine-2-carboxamide (71.8 mg, 233.43 umol, 46.05% yield, 99.419% purity) was obtained as white solid.

LCMS (ESI) m/z 306.2 [M+H]⁺; ¹H NMR (400 MHz, DMSO-δ₆) δ=12.47 (s, 1H), 8.41 (d, J=7.8 Hz, 1H), 8.27 (d, J=5.3 Hz, 1H), 7.30-7.24 (m, 2H), 4.04 (br dd, J=4.3, 8.5 Hz, 1H), 1.81-1.66 (m, 6H), 1.61-1.47 (m, 8H).

### Example 50. MPL-034

### Synthesis of 4-chloro-1H-pyrrolo[2,3-c]pyridine-2-carbonyl chloride

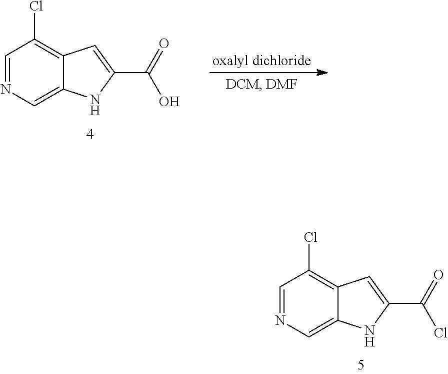 [SMILES: O=C(O)c1cc2c(Cl)cncc2[nH]1 ; O=C(Cl)c1cc2c(Cl)cncc2[nH]1]

To a solution of 4-chloro-1H-pyrrolo[2,3-c]pyridine-2-carboxylic acid (100 mg, 508.67 umol, 1 eq) in DCM (5 mL) was added oxalyl dichloride (645.64 mg, 5.09 mmol, 445.27 uL, 10 eq) and DMF (1.12 mg, 15.26 umol, 1.17 uL, 0.03 eq) under N2, The mixture was stirred at 80° C. for 0.5 hr. LC-MS showed the starting material 4 was consumed completely and one main peak with desired mass was detected. The mixture was concentrated under reduced pressure to give a residue. The crude product 4-chloro-1H-pyrrolo[2,3-c]pyridine-2-carbonyl chloride (109 mg, 506.89 umol, 99.65% yield) was obtained as yellow solid and used directly for the next step without purification. LCMS (ESI) m/z 211.0 [M-Cl+OMe]⁺

### Synthesis of 4-chloro-N-cyclooctyl-1H-pyrrolo[2,3-c]pyridine-2-carboxamide

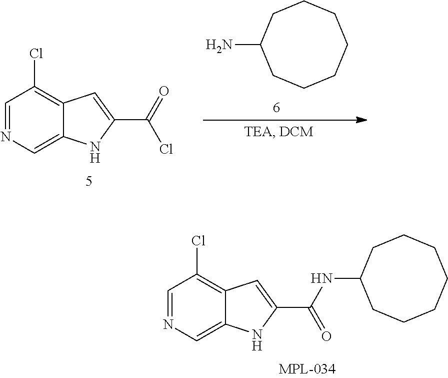 [SMILES: O=C(Cl)c1cc2c(Cl)cncc2[nH]1 ; NC1CCCCCCC1 ; O=C(NC1CCCCCCC1)c1cc2c(Cl)cncc2[nH]1]

To a solution of 4-chloro-1H-pyrrolo[2,3-c]pyridine-2-carbonyl chloride (109 mg, 506.89 umol, 1 eq) in DCM (3 mL) was added cyclooctanamine (193.47 mg, 1.52 mmol, 3 eq) and TEA (153.88 mg, 1.52 mmol, 211.66 uL, 3 eq), the mixture was stirred at 25° C. for 0.5 hr under N2. TLC and LC-MS showed the starting material 5 was consumed completely and one main peak with desired mass was detected. The reaction mixture was diluted with solvent of DCM:MeOH=10:1 (40 mL) and washed with brine (20 mL×2). Then the organic layers were dried over anhydrous Na₂SO₄, filtered and concentrated under reduced pressure to give a residue. The residue was purified by column chromatography (SiO₂, DCM:MeOH=1:0 to 50:1). The product 4-chloro-N-cyclooctyl-1H-pyrrolo[2,3-c]pyridine-2-carboxamide (44.7 mg, 145.39 umol, 28.68% yield, 99.462% purity) was obtained as yellow solid. The product was confirmed by ¹H NMR. Purity comes from LCMS.

LCMS (ESI) m/z 306.2 [M+H]⁺; ¹H NMR (400 MHz, DMSO-δ₆)=12.43 (br s, 1H), 8.72 (s, 1H), 8.62 (br d, J=7.9 Hz, 1H), 8.18 (s, 1H), 7.37 (s, 1H), 4.11-4.02 (m, 1H), 1.83-1.66 (m, 6H), 1.63-1.43 (m, 8H).

### Example 51. MPL-035

### Synthesis of 4-bromo-N-cyclooctyl-1H-pyrrolo[2,3-b]pyridine-2-carboxamide

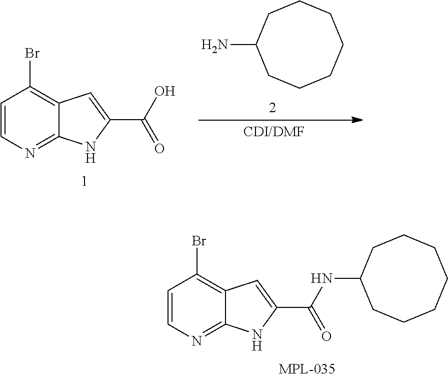 [SMILES: O=C(O)c1cc2c(Br)ccnc2[nH]1 ; NC1CCCCCCC1 ; O=C(NC1CCCCCCC1)c1cc2c(Br)ccnc2[nH]1]

To a solution of 4-bromo-1H-pyrrolo[2,3-b]pyridine-2-carboxylic acid (100 mg, 414.87 umol, 1 eq) and CDI (100.91 mg, 622.30 umol, 1.5 eq) in DMF (3 mL) the mixture was stirred at 25° C. for 30 min, then cyclooctanamine (79.17 mg, 622.30 umol, 1.5 eq) was added, the mixture was stirred at 25° C. for 12 h under N₂. TLC and LC-MS showed the starting material 1 was consumed completely and one main peak with desired mass was detected. The mixture was diluted with DCM (20 mL) and washed with water (20 mL×5) and HCl (1 M, 20 mL). The organic layers were dried over anhydrous Na₂SO₄ and concentrated under reduced pressure to give a residue. The residue was purified by column chromatography (SiO₂, DCM:MeOH=1:0 to 200:1). The product 4-bromo-N-cyclooctyl-1H-pyrrolo[2,3-b]pyridine-2-carboxamide (74.4 mg, 209.36 umol, 50.47% yield, 98.562% purity) was obtained as white solid.

LCMS (ESI) m/z 350.1 [M+H]⁺; ¹H NMR (400 MHz, DMSO-δ₆) δ=12.47 (br s, 1H), 8.43 (br d, J=7.5 Hz, 1H), 8.18 (br d, J=4.9 Hz, 1H), 7.40 (br d, J=4.6 Hz, 1H), 7.21 (br s, 1H), 4.04 (br s, 1H), 1.81-1.66 (m, 6H), 1.52 (br d, J=9.9 Hz, 8H).

### Example 52. MPL-038

### Synthesis of 4-methoxy-1H-pyrrolo[2,3-b]pyridine-2-carbonyl chloride

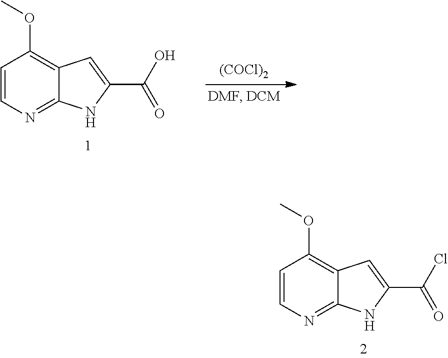

To a solution of 4-methoxy-1H-pyrrolo[2,3-b]pyridine-2-carboxylic acid (200 mg, 1.04 mmol, 1 eq) in DCM (4 mL) was added DMF (3.80 mg, 52.04 umol, 4.00 uL, 0.05 eq) and (COCl)₂ (2.90 g, 22.85 mmol, 2 mL, 21.95 eq). The mixture was stirred at 25° C. for 1 hr. LCMS showed the starting material 1 was consumed and desire product formed. The mixture was directly concentrated under reduce pressure to give a residue. The residue was directly used in next step without any purification. Compound 4-methoxy-1H-pyrrolo[2,3-b]pyridine-2-carbonyl chloride (200 mg, 902.11 umol, 86.68% yield, 95% purity) was obtained as a white solid. LCMS (ESI) m/z 206.9 [M+H]⁺

### Synthesis of N-cyclooctyl-4-methoxy-1H-pyrrolo[2,3-b]pyridine-2-carboxamide

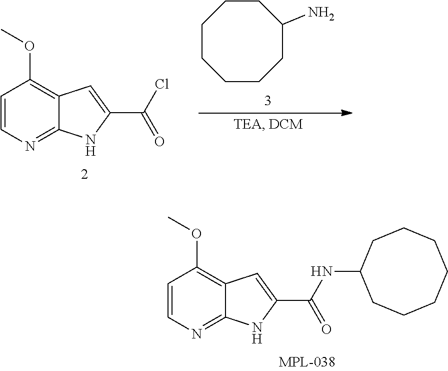 [SMILES: COc1ccnc2[nH]c(C(=O)Cl)cc12 ; NC1CCCCCCC1 ; COc1ccnc2[nH]c(C(=O)NC3CCCCCCC3)cc12]

To a solution of 4-methoxy-1H-pyrrolo[2,3-b]pyridine-2-carbonyl chloride (200 mg, 949.59 umol, 1 eq) in DCM (6 mL) was added TEA (288.27 mg, 2.85 mmol, 396.52 uL, 3 eq) and cyclooctanamine (241.63 mg, 1.90 mmol, 2 eq). The mixture was stirred at 25° C. for 2 hrs. LCMS showed the starting material 2 was consumed and desire product formed. The mixture was directly concentrated under reduce pressure to give a residue. The residue was purified by column chromatography (SiO₂, DCM:MeOH=100:1 to 20:1). Compound N-cyclooctyl-4-methoxy-1H-pyrrolo[2,3-b]pyridine-2-carboxamide (150 mg, 495.72 umol, 52.20% yield, 99.6% purity) was obtained as a white solid. LCMS (ESI) m/z 302.2 [M+H]⁺

### Example 53. MPL-040

### N-cyclooctyl-4-(methylamino)-1H-pyrrolo[2,3-b]pyridine-2-carboxamide

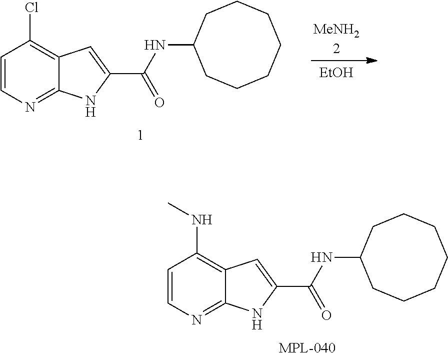 [SMILES: "MeNH2" ; "EtOH" ; O=C(NC1CCCCCCC1)c1cc2c(Cl)ccnc2[nH]1 ; CNc1ccnc2[nH]c(C(=O)NC3CCCCCCC3)cc12]

4-chloro-N-cyclooctyl-1H-pyrrolo[2,3-b]pyridine-2-carboxamide (100 mg, 327.01 umol, 1 eq) in methanamine (33.85 mg, 327.01 umol, 4 mL, 1 eq), the mixture was stirred at 120° C. for 12 hrs in a 30 mL of autoclave. TLC and LC-MS showed the starting material was consumed completely and one main peak with desired mass was detected. The reaction mixture was concentrated under reduced pressure to give a residue. The residue was purified by column chromatography (SiO₂, DCM:MeOH=1:0 to 30:1). The product N-cyclooctyl-4-(methylamino)-1H-pyrrolo[2,3-b]pyridine-2-carboxamide (41.9 mg, 135.75 umol, 41.51% yield, 97.323% purity) was obtained as white solid.

LCMS (ESI) m/z 239.0 [M+H]⁺; ¹H NMR (400 MHz, CDCl₃) δ=11.51 (br s, 1H), 7.95-7.86 (m, 2H), 7.13 (s, 1H), 6.85 (br d, J=4.8 Hz, 1H), 6.07 (d, J=5.7 Hz, 1H), 4.01 (br s, 1H), 2.84 (br d, J=4.4 Hz, 3H), 1.84-1.58 (m, 8H), 1.58-1.48 (m, 6H).

### Example 54. MPL-043

### Synthesis of 4-fluoro-6-methyl-N-[(1S,2S,3S,5R)-2,6,6-trimethylnorpinan-3-yl]-1H-pyrrolo[2,3-b]pyridine-2-carboxamide

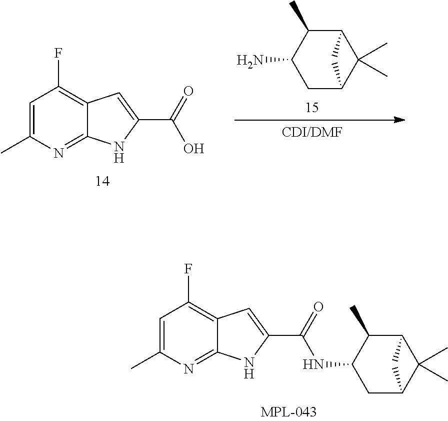

4-fluoro-6-methyl-1H-pyrrolo[2,3-b]pyridine-2-carboxylic acid (650 mg, 502.16 umol, 1 eq) and CDI (122.14 mg, 753.24 umol, 1.5 eq) was added in DMF (4 mL), the mixture was stirred at 30° C. for 0.5 h, then (1S,2S,3S,5R)-2,6,6-trimethylnorpinan-3-amine (76.96 mg, 502.16 umol, 1 eq) was added under N₂, the mixture was stirred at 30° C. for 0.5 h. LC-MS showed the starting material 14 was consumed completely and one main peak with desired mass was detected. The mixture was added in water (20 mL) and stirred for 10 mins, then filtered and the filter cake was washed with 10 mL×3 of Petroleum ether, dried under reduced pressure to give product. The residue was purified by prep-HPLC (column: Phenomenex Synergi C18 150*30 mm*4 um; mobile phase: [water (0.225% FA)-ACN]; B %: 44%-74%, 11 min). The product 4-fluoro-6-methyl-N-[(1S,2S,3S,5R)-2,6,6-trimethylnorpinan-3-yl]-1H-pyrrolo[2,3-b]pyridine-2-carboxamide (10.1 mg, 30.41 umol, 6.06% yield, 99.184% purity) was obtained as white solid.

LCMS (ESI) m/z 330.2 [M+H]⁺; ¹H NMR (400 MHz, DMSO-δ₆) δ=12.25 (br s, 1H), 8.36 (br d, J=8.3 Hz, 1H), 7.22 (s, 1H), 6.90 (d, J=11.4 Hz, 1H), 4.40-4.30 (m, 1H), 2.53 (s, 3H), 2.45-2.35 (m, 2H), 2.06 (br t, J=6.8 Hz, 1H), 1.94 (br s, 1H), 1.84-1.79 (m, 1H), 1.69 (br dd, J=5.9, 12.9 Hz, 1H), 1.23 (s, 3H), 1.18 (br d, J=9.6 Hz, 1H), 1.07-1.04 (m, 6H).

### Example 55. MPL-044

### Synthesis of N-cyclooctyl-4-fluoro-6-methyl-1H-pyrrolo[2,3-b]pyridine-2-carboxamide

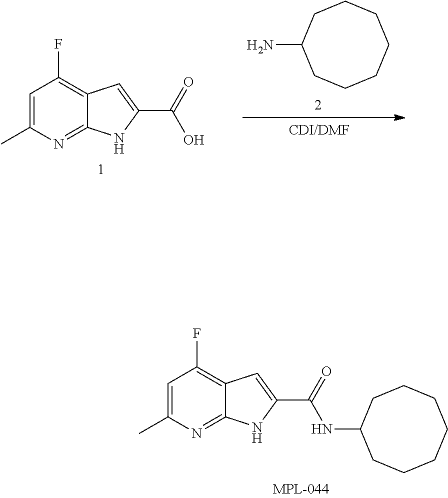 [SMILES: Cc1cc(F)c2cc(C(=O)O)[nH]c2n1 ; NC1CCCCCCC1 ; Cc1cc(F)c2cc(C(=O)NC3CCCCCCC3)[nH]c2n1]

4-fluoro-6-methyl-1H-pyrrolo[2,3-b]pyridine-2-carboxylic acid (500 mg, 386.27 umol, 1 eq) and CDI (93.95 mg, 579.41 umol, 1.5 eq) was added in DMF (3 mL), the mixture was stirred at 30° C. for 0.5 h, then cyclooctanamine (49.14 mg, 386.27 umol, 1 eq) was added under N₂, the mixture was stirred at 30° C. for 0.5 h. LC-MS showed the starting material 1 was consumed completely and one main peak with desired mass was detected. The mixture was added in water (20 mL) and stirred for 10 mins, then filtered and the filter cake was washed with 10 mL×3 of Petroleum ether, dried under reduced pressure to give product. The residue was purified by prep-HPLC (nomenex Synergi C18 150*30 mm*4 um; mobile phase: [water (0.225% FA)-ACN]; B %: 39%-68%, 11 min). The product N-cyclooctyl-4-fluoro-6-methyl-1H-pyrrolo[2,3-b]pyridine-2-carboxamide (10 mg, 32.93 umol, 8.53% yield, 99.902% purity) was obtained as white solid.

LCMS (ESI) m/z 304.1 [M+H]⁺; ¹H NMR (400 MHz, DMSO-δ₆) δ=12.21 (br s, 1H), 8.23 (br d, J=7.8 Hz, 1H), 7.19 (d, J=2.0 Hz, 1H), 6.89 (d, J=11.2 Hz, 1H), 4.09-3.96 (m, 1H), 2.53 (s, 3H), 1.82-1.66 (m, 6H), 1.60-1.44 (m, 8H).

### Example 56. MPL-045

### Synthesis of 4-chloro-6-methyl-N-[(1S,2S,3S,5R)-2,6,6-trimethylnorpinan-3-yl]-1H-pyrrolo[2,3-b]pyridine-2-carboxamide

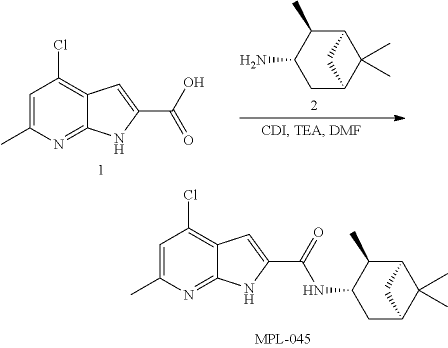

To a solution of 4-chloro-6-methyl-1H-pyrrolo[2,3-b]pyridine-2-carboxylic acid (200 mg, 949.59 umol, 1 eq) in DMF (5 mL) was added (1S,2S,3S,5R)-2,6,6-trimethylnorpinan-3-amine (291.08 mg, 1.90 mmol, 2 eq) and CDI (184.77 mg, 1.14 mmol, 1.2 eq). The mixture was stirred at 30° C. for 12 hrs. LCMS showed it was consumed completely and main desired compound. The mixture was diluted with EtOAc (30 mL). It was washed with aqueous 5% LiCl (10 mL×3), dried with anhydrous Na₂SO₄, filtered and concentrated in vacuo. The filtrate residue was purified by column chromatography (SiO₂, Petroleum ether. EtOAc=1:1). Then the residue was lyophilized. Compound 4-chloro-6-methyl-N-[(1S,2S,3S,5R)-2,6,6-trimethylnorpinan-3-yl]-1H-pyrrolo[2,3-b]pyridine-2-carboxamide (200 mg, 558.08 umol, 58.77% yield, 96.51% purity) was obtained as a white solid.

LCMS (ESI) m/z 346.1 [M+H]⁺; 1H NMR (400 MHz, CDCl₃) 9.67 (br s, 1H), 7.06 (s, 1H), 6.85 (s, 1H), 6.09 (br d, J=8.4 Hz, 1H), 4.56-4.47 (m, 1H), 2.76-2.68 (m, 1H), 2.63 (s, 3H), 2.52-2.46 (m, 1H), 2.04-1.99 (m, 1H), 1.97-1.88 (m, 2H), 1.71-1.67 (m, 1H), 1.27 (s, 3H), 1.19 (d, J=7.2 Hz, 3H), 1.10 (s, 3H), 0.95 (d, J=9.9 Hz, 1H).

### Example 57. MPL-062

### 4,5-difluoro-N-[(1S,2S,3S,5R)-2,6,6-trimethylnorpinan-3-yl]-1H-pyrrolo[2,3-b]pyridine-2-carboxamide

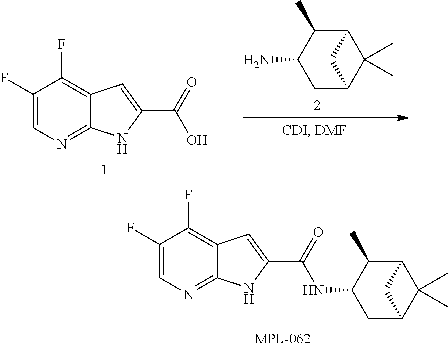 [SMILES: O=C(O)c1cc2c(F)c(F)cnc2[nH]1 ; C[C@@H]1[C@@H](N)C[C@H]2C[C@@H]1C2(C)C ; C[C@@H]1[C@@H](NC(=O)c2cc3c(F)c(F)cnc3[nH]2)C[C@H]2C[C@@H]1C2(C)C]

To a solution of 4,5-difluoro-1H-pyrrolo[2,3-b]pyridine-2-carboxylic acid (100 mg, 504.73 umol, 1 eq) in DMF (1 mL) was added CDI (98.21 mg, 605.68 umol, 1.2 eq). The mixture was stirred at 25° C. for 0.5 h. (1S,2S,3S,5R)-2,6,6-trimethylnorpinan-3-amine (92.83 mg, 605.68 umol, 1.2 eq) was added. The mixture was stirred at 25° C. for 11.5 h. LCMS showed desired compound mass was detected. TLC showed most of starting material was consumed and new spots were observed. The reaction mixture was added to water (15 mL). Then filtered and the filter cake was washed with 10 mL of water, dried in vacuo to give crude product. The residue was purified by column chromatography (SiO₂, Petroleum ether/EtOAc=1:0 to 10:1). The product 4,5-difluoro-N-[(1S,2S,3S,5R)-2,6,6-trimethylnorpinan-3-yl]-1H-pyrrolo[2,3-b]pyridine-2-carboxamide (24.4 mg, 70.56 umol, 13.98% yield, 96.4% purity) was obtained as white solid.

LCMS (ESI) m/z 334.2 [M+H]⁺; ¹H NMR (500 MHz, DMSO-d₆) δ=12.61 (br s, 1H), 8.50-8.47 (m, 1H), 7.34 (s, 1H), 4.42-4.30 (m, 1H), 2.45-2.41 (m, 1H), 2.37 (br s, 1H), 2.11-2.03 (m, 1H), 1.95 (br s, 1H), 1.82 (br s, 1H), 1.70 (br dd, J=5.3, 12.9 Hz, 1H), 1.23 (s, 3H), 1.18 (br d, J=9.3 Hz, 1H), 1.06 (br s, 6H).

### Example 58. MPL-063

### Synthesis of N-(4,4-dimethylcyclohexyl)-4,5-difluoro-1H-pyrrolo[2,3-b]pyridine-2-carboxamide

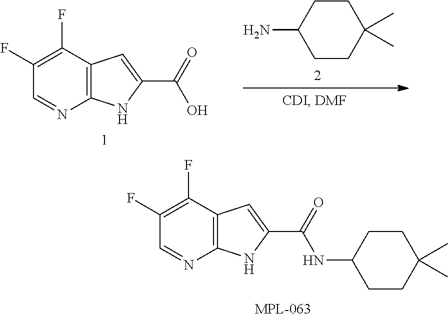

To a solution of 4,5-difluoro-1H-pyrrolo[2,3-b]pyridine-2-carboxylic acid (100 mg, 504.73 umol, 1 eq) in DMF (1 mL) was added CDI (98.21 mg, 605.67 umol, 1.2 eq). The mixture was stirred at 30° C. for 0.5 h. 4,4-dimethylcyclohexanamine (77.06 mg, 605.67 umol, 1.2 eq) was added and the mixture was stirred at 30° C. for 11.5 h. LCMS showed 27% of the starting material was still remained. The reaction mixture was added to water (15 ml), filtered and the filter cake was washed with 10 mL of water, dried in vacuo to give crude product. The crude product was purified by prep-HPLC (column: YMC-Actus Triart C18 150*30 mm*5 um; mobile phase: [water (0.225% FA)-ACN]; B %: 55%-75%, 11 min). The product N-(4,4-dimethylcyclohexyl)-4,5-difluoro-1H-pyrrolo[2,3-b]pyridine-2-carboxamide (21.2 mg, 68.98 umol, 13.67% yield, 100% purity) was obtained as white solid. Purity comes from LCMS and the product was confirmed by ¹H NMR.

LCMS (ESI) m/z 308.1 [M+H]⁺; ¹H NMR (400 MHz, DMSO-d6) δ=12.59 (br s, 1H), 8.52-8.46 (m, 1H), 8.36 (br d, J=7.8 Hz, 1H), 7.29 (s, 1H), 3.73 (br d, J=7.6 Hz, 1H), 1.68 (br d, J=9.8 Hz, 2H), 1.59-1.47 (m, 2H), 1.45-1.37 (m, 2H), 1.33-1.23 (m, 2H), 0.94 (d, J=8.1 Hz, 6H).

### Example 59. MPL-064

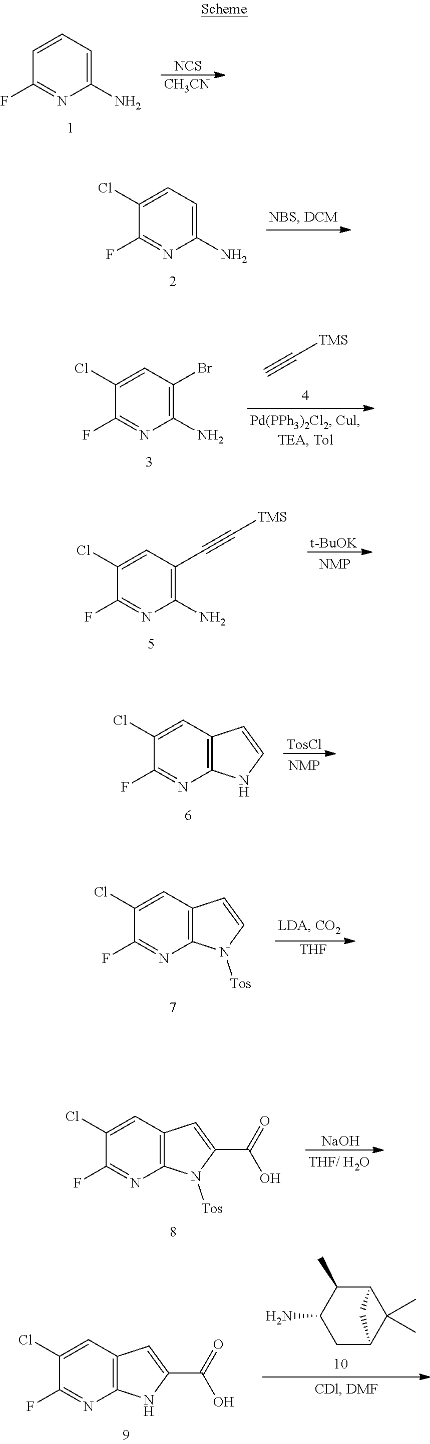

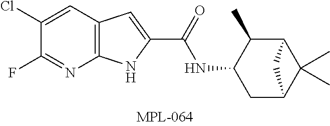 [SMILES: C[C@@H]1[C@@H](NC(=O)c2cc3cc(Cl)c(F)nc3[nH]2)C[C@H]2C[C@@H]1C2(C)C]

### Synthesis of 3-bromo-5-chloro-6-fluoro-pyridin-2-amine

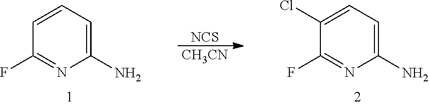

To a solution of NCS (28.59 g, 214.09 mmol, 1.2 eq) in CH₃CN (200 mL) was added 6-fluoropyridin-2-amine (20 g, 178.40 mmol, 1 eq), the mixture was stirred at 70° C. for 6 hrs. Then NCS (2 g) was added, the mixture was stirred at 70° C. for 4 h. LC-MS showed the starting material 1 was consumed completely and one main peak with desired mass was detected. The mixture was concentrated under reduced pressure to give a residue and diluted with water (100 mL), and extracted with EtOAc (150 mL×3). The organic layers were dried over anhydrous Na₂SO₄ and concentrated under reduced pressure to give a residue. The residue was purified by column chromatography (SiO₂, Petroleum ether/EtOAc=1:0 to 10:1). The product 5-chloro-6-fluoro-pyridin-2-amine (16.7 g, 102.56 mmol, 57.49% yield, 90% purity) was obtained as brown solid.

### Synthesis of 3-bromo-5-chloro-6-fluoro-pyridin-2-amine

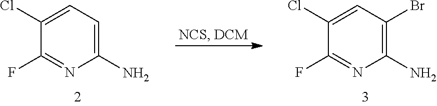

To a solution of 5-chloro-6-fluoro-pyridin-2-amine (16.7 g, 113.95 mmol, 1 eq) in DCM (200 mL) was added NBS (30.42 g, 170.93 mmol, 1.5 eq), the mixture was stirred at 25° C. for 1 hr. TLC showed the starting material 2 was consumed completely and one main spot was detected. The mixture was concentrated under reduced pressure to give a residue. The residue was purified by column chromatography (SiO₂, Petroleum ether/EtOAc=1:0 to 10:1). The product 3-bromo-5-chloro-6-fluoro-pyridin-2-amine (21.0 g, 88.49 mmol, 77.65% yield, 95% purity) was obtained as brown solid.

### Synthesis of 5-chloro-6-fluoro-3-(2-trimethylsilylethynyl)pyridin-2-amine

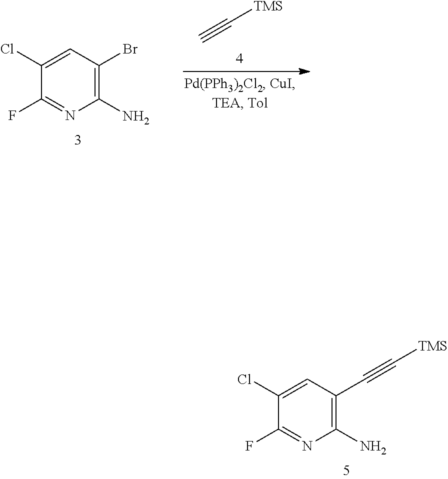

To a solution of 3-bromo-5-chloro-6-fluoro-pyridin-2-amine (10 g, 44.36 mmol, 1 eq) in TEA (80 mL) was added ethynyl(trimethyl)silane (43.57 g, 443.57 mmol, 61.45 mL, 10 eq) CuI (2.53 g, 13.31 mmol, 0.3 eq) Pd(PPh₃)₄ (2.56 g, 2.22 mmol, 0.05 eq), the mixture was stirred at 50° C. for 12 hr under N₂. LC-MS showed the starting material 3 was consumed completely and one main peak with desired mass was detected. The mixture was concentrated under reduced pressure to give a residue. The residue was purified by column chromatography (SiO₂, Petroleum ether/EtOAc=1:0 to 50:1). The product 5-chloro-6-fluoro-3-(2-trimethylsilylethynyl)pyridin-2-amine (9.2 g, 30.32 mmol, 68.35% yield, 80% purity) was obtained as brown solid. LCMS (ESI) m/z 243.1 [M+H]⁺

### Synthesis of 5-chloro-6-fluoro-1H-pyrrolo[2,3-b]pyridine

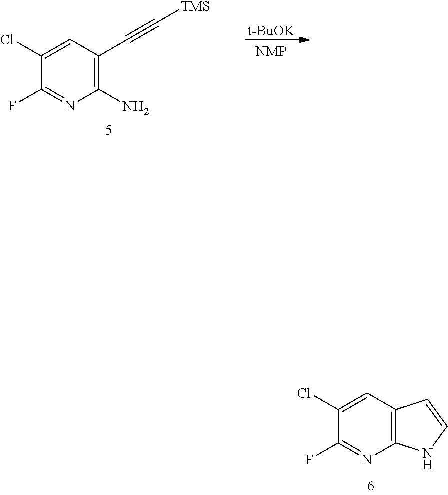 [SMILES: "t-BuOK" ; C[Si](C)(C)C#Cc1cc(Cl)c(F)nc1N ; Fc1nc2[nH]ccc2cc1Cl]

To a solution of 5-chloro-6-fluoro-3-(2-trimethylsilylethynyl)pyridin-2-amine (9.2 g, 37.90 mmol, 1 eq) in NMP (100 mL) was added KOtBu (12.76 g, 113.70 mmol, 3 eq) under N₂. The mixture was stirred at 80° C. for 12 hrs. LCMS showed starting material 5 was consumed completely. The mixture as crude product was used directly for the next step.

LCMS (ESI) m/z 171.0 [M+H]⁺

### Synthesis of 5-chloro-6-fluoro-1-(p-tolylsulfonyl)pyrrolo[2,3-b]pyridine

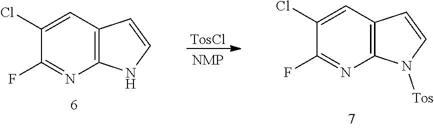

To a solution of 5-chloro-6-fluoro-1H-pyrrolo[2,3-b]pyridine (6.46 g, 37.87 mmol, 1 eq) in NMP (80 mL) was added TosCl (14.44 g, 75.75 mmol, 2 eq) under N₂. The mixture was stirred at 25° C. for 12 hr. LCMS showed starting material 6 was consumed completely and one main peak with desired mass was detected. The reaction mixture was added to water (500 mL) and extracted with (200 mL×4). The organic layers were dried over anhydrous Na₂SO₄ and concentrated under reduced pressure to give a residue. The residue was purified by column chromatography (SiO₂, Petroleum ether/EtOAc=1/0 to 10/1). The product 5-chloro-6-fluoro-1-(p-tolylsulfonyl) pyrrolo[2,3-b]pyridine (2.4 g, 7.02 mmol, 18.54% yield, 95% purity) was obtained as white solid.

### Synthesis of 5-chloro-6-fluoro-1-(p-tolylsulfonyl)pyrrolo[2,3-b]pyridine-2-carboxylic acid

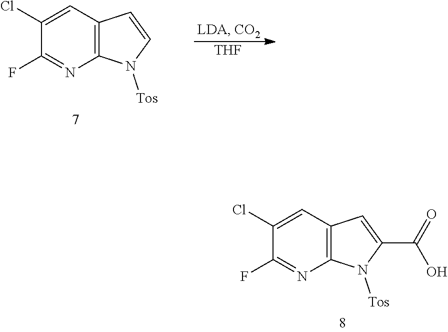

LDA (2 M, 4.06 mL, 1.1 eq) was added to a solution of 5-chloro-6-fluoro-1-(p-tolylsulfonyl) pyrrolo[2,3-b]pyridine (2.4 g, 7.39 mmol, 1 eq) in THE (50 mL) at −70° C. under N₂, then the mixture was stirred for 1 h, then CO₂ (325.24 mg, 7.39 mmol, 1 eq) was added, the mixture was stirred at −70° C. for 0.5 h. LC-MS showed desired product was detected. The reaction mixture was quenched by addition saturated aqueous NH₄Cl (30 mL) at −70° C., and then concentrated under reduced pressure to remove THE and then diluted with 50 mL water, filtered and the filter cake was washed with 50 mL of water, dried under reduced pressure to give the crude product. The residue was purified by column chromatography (SiO₂, Petroleum ether/EtOAc=1:0 to 1:1 contained 1% AcOH). The product 5-chloro-6-fluoro-1-(p-tolylsulfonyl)pyrrolo[2,3-b]pyridine-2-carboxylic acid (800 mg, 2.06 mmol, 27.89% yield, 95% purity) was obtained as yellow solid.

### Synthesis of 5-chloro-6-fluoro-1H-pyrrolo[2,3-b]pyridine-2-carboxylic acid

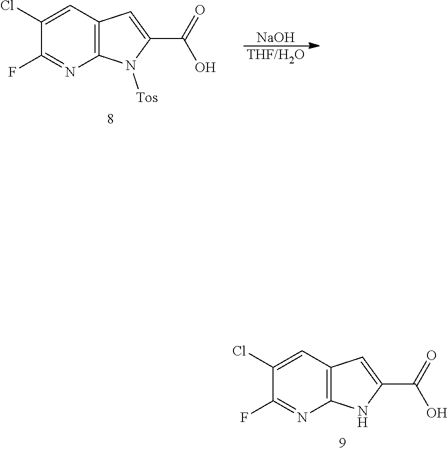

To a solution of 5-chloro-6-fluoro-1-(p-tolylsulfonyl)pyrrolo[2,3-b]pyridine-2-carboxylic acid (750 mg, 2.03 mmol, 1 eq) and NaOH (2 M, 4.29 mL, 4.22 eq) in THE (4 mL), the mixture was stirred at 75° C. for 3 hr. LC-MS showed the starting material 8 was consumed completely. The mixture was concentrated under reduced pressure to remove the THF, then acidified with HCl (2 M) to pH=5. The mixture was filtered and the filter cake was washed with 10 mL×3 of Petroleum ether, dried under reduced pressure to give the product. The product 5-chloro-6-fluoro-1H-pyrrolo[2,3-b]pyridine-2-carboxylic acid (350 mg, 1.55 mmol, 76.19% yield, 95% purity) was obtained as white solid.

### Synthesis of 5-chloro-6-fluoro-N-[(1S,2S,3S,5R)-2,6,6-trimethylnorpinan-3-yl]-1H-pyrrolo[2,3-b]pyridine-2-carboxamide

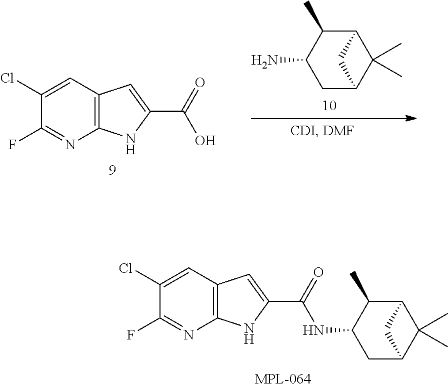 [SMILES: O=C(O)c1cc2cc(Cl)c(F)nc2[nH]1 ; C[C@@H]1[C@@H](N)C[C@H]2C[C@@H]1C2(C)C ; C[C@@H]1[C@@H](NC(=O)c2cc3cc(Cl)c(F)nc3[nH]2)C[C@H]2C[C@@H]1C2(C)C]

To a solution of 5-chloro-6-fluoro-1H-pyrrolo[2,3-b]pyridine-2-carboxylic acid (175 mg, 815.54 umol, 1 eq) and CDI (198.36 mg, 1.22 mmol, 1.5 eq) in DMF (2 mL) the mixture was stirred at 25° C. for 30 min, then (1S,2S,3S,5R)-2,6,6-trimethylnorpinan-3-amine (187.49 mg, 1.22 mmol, 1.5 eq) was added, the mixture was stirred at 25° C. for 0.5 h under N₂. LC-MS showed one main peak with desired mass was detected. The mixture was added to a solution of LiCl (100 ml, 3%), filtered and the filter cake was washed with 20 mL of water, dried under reduced pressure to give residue. The residue was purified by column chromatography (SiO₂, Petroleum ether/EtOAc=1/0 to 10/1). The product 5-chloro-6-fluoro-N-[(1S,2S,3S,5R)-2,6,6-trimethylnorpinan-3-yl]-1H-pyrrolo[2,3-b]pyridine-2-carboxamide (126.3 mg, 359.79 umol, 44.12% yield, 99.655% purity) was obtained as white solid.

LCMS (ESI) m/z 350.1 [M+H]⁺; ¹H NMR (400 MHz, DMSO-δ₆) δ=9.82 (br s, 1H), 8.06 (d, J=8.8 Hz, 1H), 6.77 (d, J=2.2 Hz, 1H), 6.05 (br d, J=8.3 Hz, 1H), 4.55 (br s, 1H), 2.79-2.70 (m, 1H), 2.53-2.45 (m, 1H), 2.03 (br d, J=2.6 Hz, 1H), 1.96-1.88 (m, 2H), 1.68 (br dd, J=2.2, 6.1 Hz, 1H), 1.27 (s, 3H), 1.19 (d, J=7.0 Hz, 3H), 1.12 (s, 3H), 0.93 (d, J=9.6 Hz, 1H).

### Example 60. MPL-065

### Synthesis of 5-chloro-N-(4,4-dimethylcyclohexyl)-6-fluoro-1H-pyrrolo[2,3-b]pyridine e-2-carboxamide

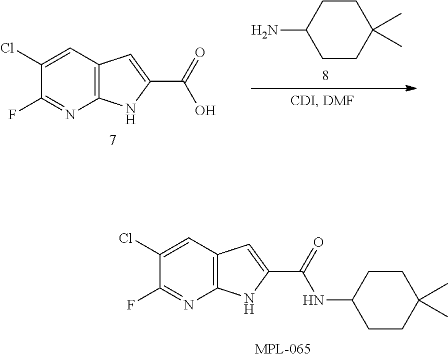

To a solution of 5-chloro-6-fluoro-1H-pyrrolo[2,3-b]pyridine-2-carboxylic acid (200 mg, 932.05 umol, 1 eq) in DMF (2 mL) was added CDI (226.70 mg, 1.40 mmol, 1.5 eq), the mixture was stirred at 30° C. for 0.5 h, then 4,4-dimethylcyclohexanamine (177.87 mg, 1.40 mmol, 1.5 eq) was added and the mixture was stirred at 30° C. for another 0.5 h. TLC and LC-MS showed the starting material 7 was consumed completely and one main peak with desired mass was detected. The mixture was added to water (20 mL), and stirred for 10 min, filtered and the filter cake was dried under reduced pressure to give the crude product. The crude product was purified by column chromatography (SiO2, DCM:MeOH=1:0 to 500:1). The product 5-chloro-N-(4,4-dimethylcyclohexyl)-6-fluoro-1H-pyrrolo[2,3-b]pyridine-2-carboxamide (89.2 mg, 271.55 umol, 29.13% yield, 98.572% purity) was obtained as white solid. Purity comes from LCMS. The product was confirmed by ¹H NMR.

LCMS (ESI) m/z 324.1 [M+H]⁺; ¹H NMR (400 MHz, DMSO-δ₆)=12.48 (s, 1H), 8.49 (d, J=9.5 Hz, 1H), 8.34 (d, J=7.9 Hz, 1H), 7.18 (d, J=1.8 Hz, 1H), 3.77-3.67 (m, 2H), 1.71-1.63 (m, 2H), 1.52 (br d, J=14.6 Hz, 2H), 1.40 (br d, J=12.5 Hz, 2H), 1.31-1.23 (m, 2H), 0.93 (d, J=9.8 Hz, 6H).

### Example 61. MPL-065A

### Synthesis of 5-chloro-N-cyclooctyl-6-fluoro-1H-pyrrolo[2,3-b]pyridine-2-carboxamide

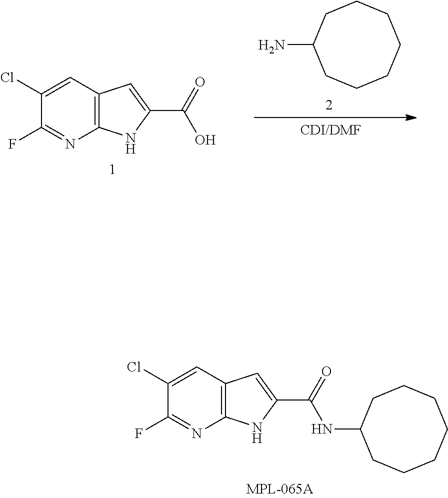 [SMILES: O=C(O)c1cc2cc(Cl)c(F)nc2[nH]1 ; NC1CCCCCCC1 ; O=C(NC1CCCCCCC1)c1cc2cc(Cl)c(F)nc2[nH]1]

To a solution of 5-chloro-6-fluoro-1H-pyrrolo[2,3-b]pyridine-2-carboxylic acid (175 mg, 815.54 umol, 1 eq) and CDI (198.36 mg, 1.22 mmol, 1.5 eq) in DMF (2 mL) the mixture was stirred at 25° C. for 30 min, then cyclooctanamine (155.64 mg, 1.22 mmol, 1.5 eq) was added. The mixture was stirred at 25° C. for 0.5 h under N₂. LC-MS showed one main peak with desired mass was detected. The mixture was added to a solution of LiCl (100 ml, 3%), filtered and the filter cake was washed with 20 mL of water, dried under reduced pressure to give residue. The residue was purified by column chromatography (SiO₂, Petroleum ether/EtOAc=1/0 to 10/1). The product 5-chloro-N-cyclooctyl-6-fluoro-1H-pyrrolo[2,3-b]pyridine-2-carboxamide (51.3 mg, 157.03 umol, 19.25% yield, 99.113% purity) was obtained as white solid.

LCMS (ESI) m/z 324.1 [M+H]⁺; ¹H NMR (400 MHz, CHLOROFORM-d) 6=9.57 (br s, 1H), 8.05 (d, J=8.8 Hz, 1H), 6.72 (d, J=2.2 Hz, 1H), 6.07 (br d, J=8.3 Hz, 1H), 4.23 (br s, 1H), 2.00-1.93 (m, 2H), 1.78-1.61 (m, 12H), 1.77-1.54 (m, 1H).

### Example 62. MPL-066

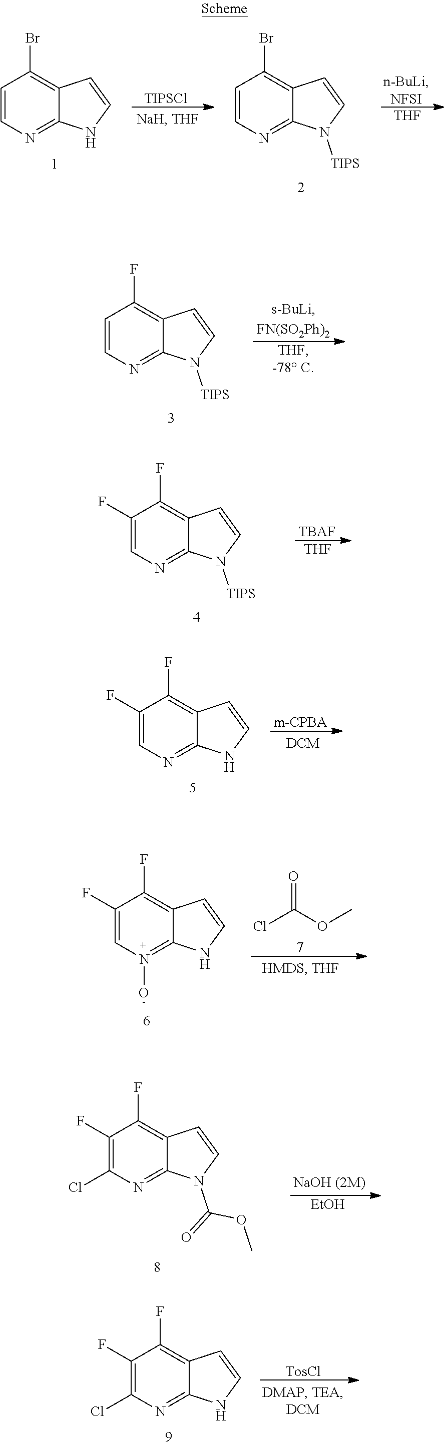

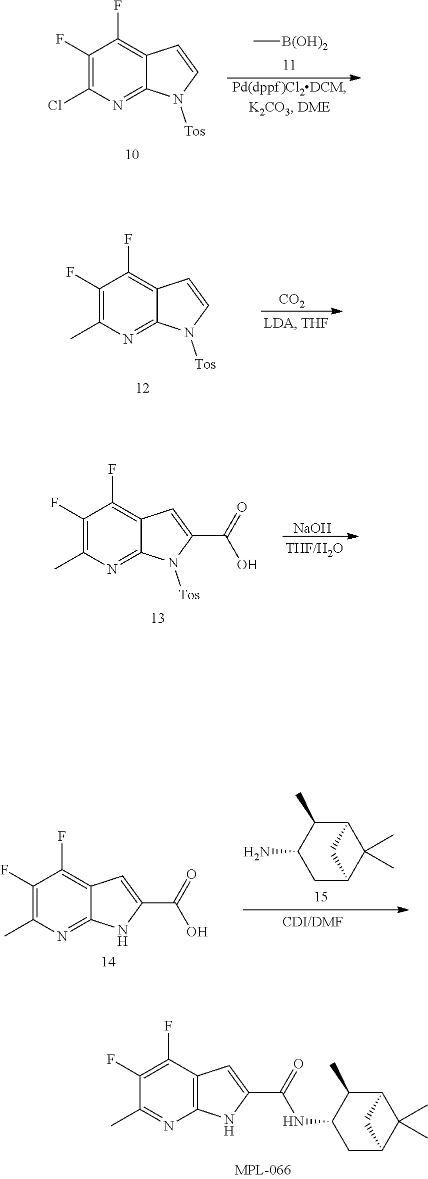

### Synthesis of 4,5-difluoro-1H-pyrrolo[2,3-b]pyridine

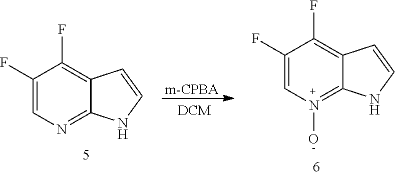

To a solution of 4,5-difluoro-1H-pyrrolo[2,3-b]pyridine (3.2 g, 20.76 mmol, 1 eq) in DCM (30 mL) was dropwise added m-CPBA (17.92 g, 83.05 mmol, 80% purity, 4 eq) in THF (20 mL) at 0° C. The mixture was stirred at 15° C. for 12 hr. LCMS showed it were main starting material. The mixture was quenched by the addition of the saturated Na₂SO₃ (5 mL), then dropwise added saturated Na₂CO₃. The mixture was extracted with Dichloromethane:Methanol (V:V=10:1), dried with anhydrous Na₂SO₄, filtered and concentrated in vacuo. The residue was purified by column chromatography (SiO₂, Petroleum ether:EtOAc=5:2 to Dichloromethane:Methanol=10:1). Compound 4,5-difluoro-7-oxido-1H-pyrrolo[2,3-b]pyridin-7-ium (3.35 g, 15.75 mmol, 75.87% yield, 80% purity) was obtained as a white solid.

### Synthesis of methyl 6-chloro-4,5-difluoro-pyrrolo[2,3-b]pyridine-1-carboxylate

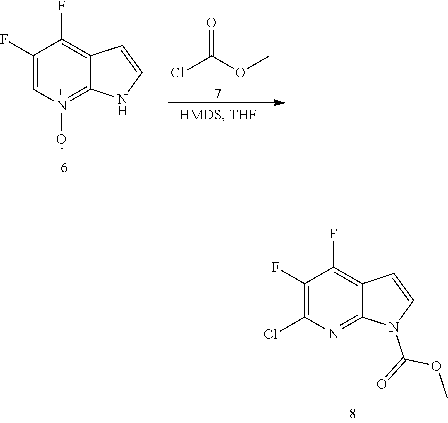

To a solution of 4,5-difluoro-7-oxido-1H-pyrrolo[2,3-b]pyridin-7-ium (2.8 g, 16.46 mmol, 1 eq) in THE (30 mL) was added HMDS (2.66 g, 16.46 mmol, 3.45 mL, 1 eq) and methyl carbonochloridate (4.67 g, 49.38 mmol, 3.82 mL, 3 eq) (9.55 g) at 0° C. The mixture was stirred at 10° C. for 12 hr. LCMS showed there was no starting material. The mixture was concentrated in reduced pressure until without THF. The residue was added saturated NaHCO₃ (20 mL). The aqueous phase was extracted with EtOAc (20 mL×3). The combined organic phase was washed with saturated brine (10 mL×2), dried with anhydrous Na₂SO₄, filtered. The filtrate was concentrated in vacuo. The residue was purified by column chromatography (SiO₂, Petroleum ether:EtOAc=1:0 to 5:1). Compound methyl 6-chloro-4,5-difluoro-pyrrolo[2,3-b]pyridine-1-carboxylate (2.31 g, 6.56 mmol, 39.84% yield, 70% purity) was obtained white solid.

### Synthesis of 6-chloro-4,5-difluoro-1H-pyrrolo[2,3-b]pyridine

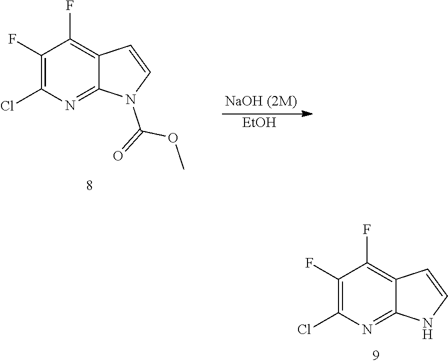 [SMILES: "EtOH" ; COC(=O)n1ccc2c(F)c(F)c(Cl)nc21 ; Fc1c(Cl)nc2[nH]ccc2c1F]

To a solution of methyl 6-chloro-4,5-difluoro-pyrrolo[2,3-b]pyridine-1-carboxylate (2.31 g, 9.37 mmol, 1 eq) in THE (11 mL) was added NaOH (2 M, 11 mL, 2.35 eq). The mixture was stirred at 10° C. for 12 hr. LCMS showed there was no starting material. The mixture was concentrated in reduced pressure until without THF. The mixture was extracted with EtOAc (10×3 mL), the organic phase was dried with anhydrous Na₂SO₄, filtered and concentrated in vacuo. The residue was used directly for next step without further purification. Compound 6-chloro-4,5-difluoro-1H-pyrrolo[2,3-b]pyridine (1.460 g, 4.65 mmol, 49.59% yield, 60% purity) was obtained as a white solid.

### Synthesis of 6-chloro-4,5-difluoro-1-(p-tolylsulfonyl)pyrrolo[2,3-b]pyridine

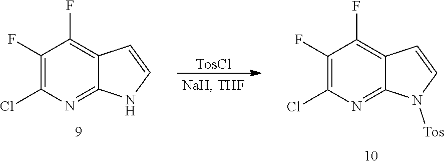

To a solution of NaH (1.38 g, 34.47 mmol, 60% purity, 10 eq) in THF (10 mL) was added a solution of 6-chloro-4,5-difluoro-1H-pyrrolo[2,3-b]pyridine (650 mg, 3.45 mmol, 1 eq) in THF (10 mL) at 0° C. under N₂, then 4-methylbenzenesulfonyl chloride (1.97 g, 10.34 mmol, 3 eq) was added at 0° C. under N₂. The mixture was stirred at 10° C. for 12 hrs under N₂ atmosphere. TLC (Petroleum ether:EtOAc=5:1) showed there was no starting material. The reaction was quenched at −0° C. with saturated aqueous NH₄Cl (5 mL). The aqueous phase was extracted with EtOAc (20 mL×3). The combined hexane phases were dried over anhydrous Na₂SO₄ and concentrated under reduced pressure to give a residue. The residue was purified by column chromatography (SiO₂, Petroleum ether:EtOAc=1:0 to 5:1). The product 6-chloro-4,5-difluoro-1-(p-tolylsulfonyl)pyrrolo[2,3-b]pyridine (1.1 g, 2.89 mmol, 83.79% yield, 90% purity) was obtained as a white solid.

### Synthesis of 6-chloro-4,5-difluoro-1H-pyrrolo[2,3-b]pyridine

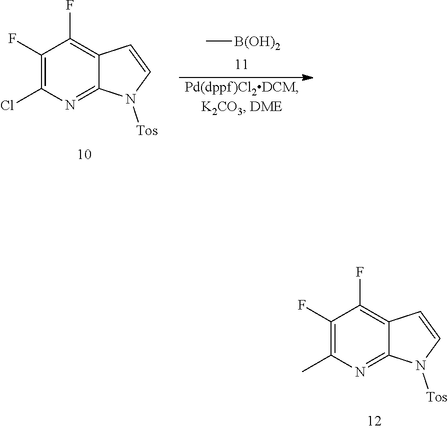

To a solution of 6-chloro-4,5-difluoro-1-(p-tolylsulfonyl)pyrrolo[2,3-b]pyridine (1.22 g, 3.56 mmol, 1 eq), methylboronic acid (1.07 g, 17.80 mmol, 5 eq), K₂CO₃ (1.48 g, 10.68 mmol, 3 eq) and Pd(dppf)Cl₂ DCM (260.45 mg, 355.95 umol, 0.1 eq) was added in DME (30 mL), the mixture was stirred at 110° C. for 12 hr under N₂. LCMS showed the starting material was consumed completely. The reaction mixture was concentrated under reduced pressure to give a residue. The residue was purified by column chromatography (SiO₂, Petroleum ether:EtOAc=1:0 to 1:1). The product 4,5-difluoro-6-methyl-1-(p-tolylsulfonyl)pyrrolo[2,3-b]pyridine (850.27 mg, 2.37 mmol, 66.70% yield, 90% purity) was obtained as white solid.

### Synthesis of 4,5-difluoro-6-methyl-1-(p-tolylsulfonyl)pyrrolo[2,3-b]pyridine-2-carboxalic acid

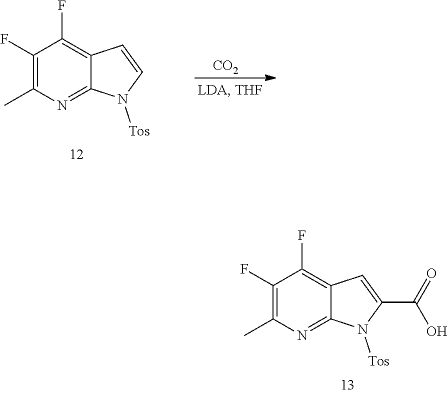

To a solution of 4,5-difluoro-6-methyl-1-(p-tolylsulfonyl)pyrrolo[2,3-b]pyridine (500 mg, 1.55 mmol, 1 eq) in THF (5 mL) at −78° C. under N₂ was treated dropwise with LDA (2 M, 1.16 mL, 1.5 eq). The reaction was stirred for 1.5 h. The mixture was stirred for 10.5 h at 10° C. under CO₂ (68.27 mg, 1.55 mmol, 1 eq). LCMS showed there was no starting material and main desired compound. The reaction was quenched at −78° C. with saturated aqueous NH₄Cl (5 mL). The aqueous phase was extracted with EtOAc (5 mL×3). The combined hexane phases were dried over anhydrous Na₂SO₄ and concentrated under reduced pressure to give a residue. The residue was used directly for next step without further purification. The product 4,5-difluoro-6-methyl-1-(p-tolylsulfonyl)pyrrolo[2,3-b]pyridine-2-carboxylic acid (427 mg, 699.35 umol, 45.08% yield, 60% purity) was obtained as white oil.

### Synthesis of 4,5-difluoro-6-methyl-1H-pyrrolo[2,3-b]pyridine-2-carboxylic acid

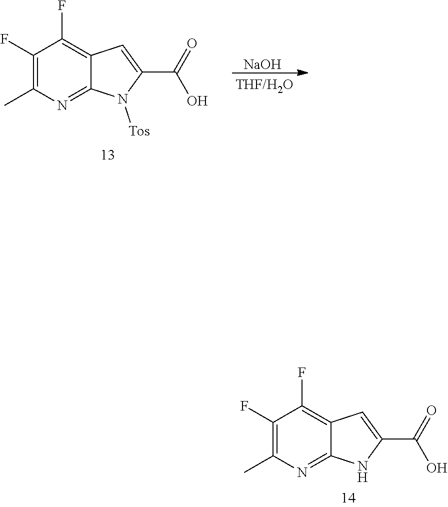

To a solution of 4,5-difluoro-6-methyl-1-(p-tolylsulfonyl)pyrrolo[2,3-b]pyridine-2-carboxylic acid (100 mg, 272.97 umol, 1 eq) in THE (1 mL) was dropwise added TBAF (1 M, 818.91 uL, 3 eq). The mixture was stirred at 80° C. for 12 hr. LCMS showed there was no starting material. The mixture was concentrated in reduced pressure until without THF. The residue was dissolved with EtOAc (20 mL). The organic phase was washed saturated brine (20 mL×3), dried with anhydrous Na₂SO₄, filtered and concentrated in vacuo. The residue was used directly for next step without further purification. Compound 4,5-difluoro-6-methyl-1H-pyrrolo[2,3-b]pyridine-2-carboxylic acid (26 mg, 91.91 umol, 33.67% yield, 75% purity) was obtained as a white solid.

### Synthesis of 4,5-difluoro-6-methyl-N-[(1S,2S,3S,5R)-2,6,6-trimethylnorpinan-3-yl]-1H-pyrrolo[2,3-b]pyridine-2-carboxamide

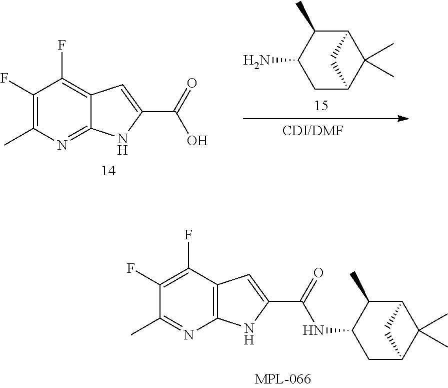 [SMILES: Cc1nc2[nH]c(C(=O)O)cc2c(F)c1F ; C[C@@H]1[C@@H](N)C[C@H]2C[C@@H]1C2(C)C ; Cc1nc2[nH]c(C(=O)N[C@H]3C[C@H]4C[C@@H]([C@@H]3C)C4(C)C)cc2c(F)c1F]

To a solution of 4,5-difluoro-6-methyl-1H-pyrrolo[2,3-b]pyridine-2-carboxylic acid (46 mg, 216.82 umol, 1 eq) in DMF (1.5 mL) was added CDI (38.67 mg, 238.51 umol, 1.1 eq). The mixture was stirred at 30° C. for 0.5 h. Then (1S,2S,3S,5R)-2,6,6-trimethylnorpinan-3-amine (34.89 mg, 227.67 umol, 1.05 eq) was added. The mixture was stirred at 30° C. for 11.5 h. LCMS showed there was no starting material. The reaction was added dropwise to H₂O (20 mL). There was much precipitation which was collected by filter. The cake was transferred in bottom flask. The crude product was purified by prep-TLC (SiO₂, Petroleum ether. EtOAc=5:1). Compound 4,5-difluoro-6-methyl-N-[(1S,2S,3S,5R)-2,6,6-trimethylnorpinan-3-yl]-1H-pyrrolo[2,3-b]pyridine-2-carboxamide (6.4 mg, 18.42 umol, 8.50% yield, 100% purity) was obtained as a white solid.

LCMS (ESI), m/z 348.4[M+H]⁺; 1H NMR (400 MHz, CHLOROFORM-d) δ=9.92 (br s, 1H), 6.86 (s, 1H), 6.12 (br d, J=7.4 Hz, 1H), 4.61-4.45 (m, 1H), 2.77-2.68 (m, 1H), 2.64 (br d, J=2.7 Hz, 3H), 2.47 (br s, 1H), 2.05-1.88 (m, 3H), 1.81-1.62 (m, 2H), 1.26 (s, 3H), 1.19 (br d, J=7.0 Hz, 3H), 1.10 (s, 3H).

### Example 63. MPL-067

### Synthesis of 4-chloro-N-[(1S,2S,3S,5R)-2,6,6-trimethylnorpinan-3-yl]-1H-pyrrolo[2,3-c]Pyridine-2-carboxamide

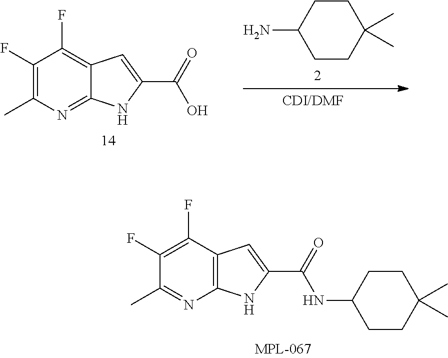

To a solution of 4,5-difluoro-6-methyl-1H-pyrrolo[2,3-b]pyridine-2-carboxylic acid (40 mg, 188.54 umol, 1 eq) in DMF (1 mL) was added CDI (33.63 mg, 207.40 umol, 1.1 eq). The mixture was stirred at 30° C. for 0.5 h. Then 4,4-dimethylcyclohexanamine (26.39 mg, 207.40 umol, 1.1 eq) was added. The mixture was stirred at 30° C. for 11.5 h. LCMS showed there was no starting material. The reaction was added dropwise to H₂O (20 mL). There was much precipitation which was collected by filter. The cake was transferred in bottom flask. The crude product was purified by prep-TLC (SiO₂, Petroleum ether. EtOAc=5:1). Compound N-(4,4-dimethylcyclohexyl)-4,5-difluoro-6-methyl-1H-pyrrolo[2,3-b]pyridine-2-carboxamide (8.4 mg, 25.96 umol, 13.77% yield, 99.326% purity) was obtained as a white solid.

LCMS (ESI), m/z 322.[M+H]⁺; ¹H NMR (400 MHz, DMSO-d6) δ=12.39 (br s, 1H), 8.30 (d, J=7.8 Hz, 1H), 7.23 (s, 1H), 3.72 (br d, J=7.4 Hz, 1H), 2.54-2.52 (m, 3H), 1.67 (br d, J=9.4 Hz, 2H), 1.58-1.46 (m, 2H), 1.45-1.37 (m, 2H), 1.29 (br d, J=12.5 Hz, 2H), 0.93 (d, J=8.2 Hz, 6H).

### Example 64. MPL-070

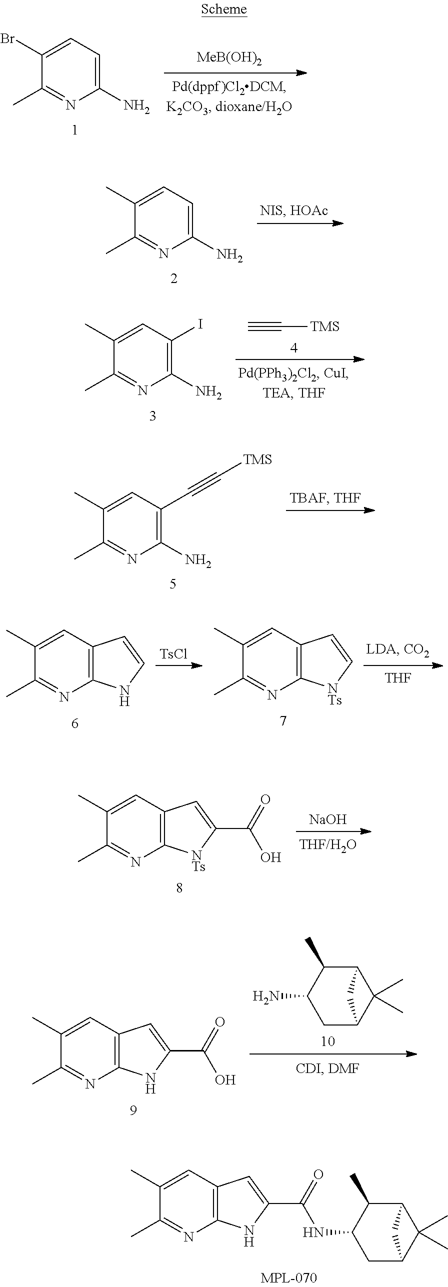

### Synthesis of 5,6-dimethyl-1-tosyl-1H-pyrrolo[2,3-b]pyridine

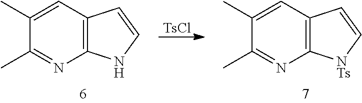

To a solution of 5,6-dimethyl-1H-pyrrolo[2,3-b]pyridine (1.31 g, 8.96 mmol, 1 eq) in NMP (20 mL) was added 4-methylbenzenesulfonyl chloride (2.05 g, 10.75 mmol, 1.2 eq). The mixture was stirred at 20° C. for 3 hr. TLC indicated Reactant 6 was consumed completely and many new spots formed. The mixture was quenched with water (100 mL) and extracted with EtOAc (100 mL×3). The organic layer was dried over Na₂SO₄, filtered and concentrated under reduced pressure. The residue was purified by flash silica gel chromatography (ISCO®; 40 g SepaFlash® Silica Flash Column, Eluent of 0˜30% EtOAc/Petroleum ether gradient at 40 mL/min). Compound 5,6-dimethyl-1-(p-tolylsulfonyl)pyrrolo[2,3-b]pyridine (1.5 g, 4.99 mmol, 55.73% yield) was obtained as a white solid.

### Synthesis of 5,6-dimethyl-1-tosyl-1H-pyrrolo[2,3-b]pyridine-2-carboxylic acid

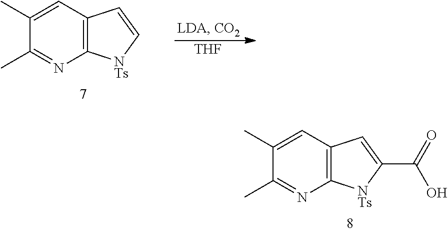

To a solution of 5,6-dimethyl-1-(p-tolylsulfonyl)pyrrolo[2,3-b]pyridine (1.5 g, 4.99 mmol, 1 eq) in THE (50 mL) was added dropwise LDA (2 M, 2.62 mL, 1.05 eq) at −78° C. After addition, the mixture was stirred at this temperature for 1 hr under N₂ atmosphere, and then the resulting mixture was stirred at −78° C. for 2 hr under CO₂ atmosphere (15 psi). LC-MS showed Reactant 7 was consumed completely and one main peak with desired mass was detected. The mixture was quenched with NH₄Cl (100 mL) and extracted with EtOAc (100 mL×3). The organic layer was dried over Na₂SO₄, filtered and concentrated under reduced pressure. The residue was purified by flash silica gel chromatography (ISCO®; 40 g SepaFlash® Silica Flash Column, Eluent of 0˜5% MeOH/DCM at 40 mL/min). Compound 5,6-dimethyl-1-(p-tolylsulfonyl)pyrrolo[2,3-b]pyridine-2-carboxylic acid (1.3 g, 3.77 mmol, 75.59% yield) was obtained as a white solid.

### Synthesis of 5,6-dimethyl-1H-pyrrolo[2,3-b]pyridine-2-carboxylic acid

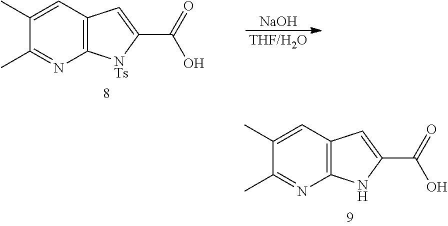 [SMILES: Cc1ccc(S(=O)(=O)n2c(C(=O)O)cc3cc(C)c(C)nc32)cc1 ; "NaOH" ; "THF/H2O" ; Cc1cc2cc(C(=O)O)[nH]c2nc1C]

To a solution of 5,6-dimethyl-1-(p-tolylsulfonyl)pyrrolo[2,3-b]pyridine-2-carboxylic acid (1.1 g, 3.19 mmol, 1 eq) in EtOH (12 mL) was added NaOH (2 M, 12 mL, 7.51 eq), the resulting mixture was stirred at 80° C. for 12 hr. LC-MS showed Reactant 8 was consumed completely and one main peak with desired mass was detected. The mixture was acidified until the precipitate was formed and filtered. The filter cake was washed with MeCN (10 mL) and filtered; the filter cake was dried to give the product. The crude was used directly in the next step without further purification. The crude product 5,6-dimethyl-1H-pyrrolo[2,3-b]pyridine-2-carboxylic acid (0.52 g, 2.62 mmol, 82.17% yield, 96% purity) as a pale solid was used into the next step without further purification.

### Synthesis of 5,6-dimethyl-N-((1S,2S,3S,5R)-2,6,6-trimethylbicyclo[3.1.1]heptan-3-yl)-1H-pyrrolo[2,3-b]pyridine-2-carboxamide

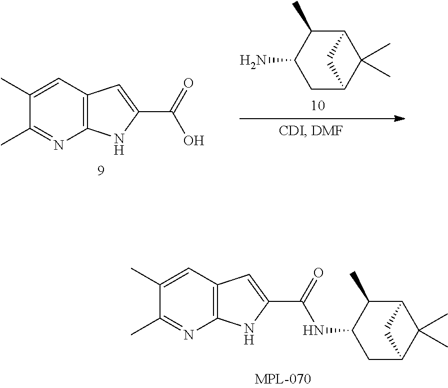

To a solution of 5,6-dimethyl-1H-pyrrolo[2,3-b]pyridine-2-carboxylic acid (0.15 g, 788.65 umol, 1 eq) in DMF (3 mL) was added CDI (191.82 mg, 1.18 mmol, 1.5 eq) and (1S,2S,3S,5R)-2,6,6-trimethylnorpinan-3-amine (145.05 mg, 946.38 umol, 1.2 eq). The mixture was stirred at 20° C. for 2 hr. LC-MS showed Reactant 9 was consumed completely and one main peak with desired mass was detected. The mixture was quenched with water (50 mL) and filtered. The filter cake was washed with MeOH (10 mL) and filtered. The filter cake was dried to give the product. Compound 5,6-dimethyl-N-[(1S,2S,3S,5R)-2,6,6-trimethylnorpinan-3-yl]-1H-pyrrolo[2,3-b]pyridine-2-carboxamide (180 mg, 553.09 umol, 70.13% yield, 100% purity) was obtained as a white solid.

LCMS (ESI) m/z 326.2 [M+H]⁺; ¹H NMR (500 MHz, DMSO-d₆) δ=11.73 (s, 1H), 8.25 (d, J=8.5 Hz, 1H), 7.77 (s, 1H), 7.04 (d, J=2.1 Hz, 1H), 4.44-4.29 (m, 1H), 2.48 (s, 3H), 2.44 (br d, J=2.1 Hz, 1H), 2.39-2.35 (m, 1H), 2.32 (s, 3H), 2.07 (br t, J=6.7 Hz, 1H), 1.95 (br d, J=2.7 Hz, 1H), 1.82 (br t, J=5.2 Hz, 1H), 1.73-1.63 (m, 1H), 1.24 (s, 3H), 1.20 (d, J=9.5 Hz, 1H), 1.11-1.04 (m, 6H);

### Example 65. MPL-071

### Synthesis of N-cyclooctyl-5,6-dimethyl-1H-pyrrolo[2,3-b]pyridine-2-carboxamide

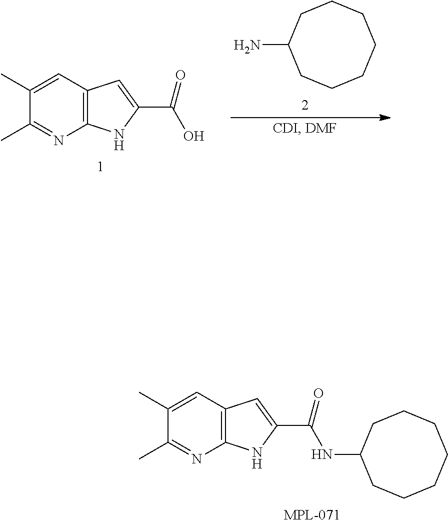 [SMILES: Cc1cc2cc(C(=O)O)[nH]c2nc1C ; NC1CCCCCCC1 ; Cc1cc2cc(C(=O)NC3CCCCCCC3)[nH]c2nc1C]

To a solution of 5,6-dimethyl-1H-pyrrolo[2,3-b]pyridine-2-carboxylic acid (100 mg, 525.77 umol, 1 eq) in DMF (1 mL) was added CDI (110.83 mg, 683.50 umol, 1.3 eq). The mixture was stirred at 30° C. for 0.5 h. cyclooctanamine (86.96 mg, 683.50 umol, 1.3 eq) was added and the reaction mixture was stirred at 30° C. for 12 h. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. The crude product was purified by silica column chromatography (eluent of 20-80% EtOAc/Petroleum ether gradient, 4 g silica column). All fractions found to contain product by TLC (Petroleum ether:EtOAc=1:1, Rf=0.3) were combined and evaporated. Compound N-cyclooctyl-5,6-dimethyl-1H-pyrrolo[2,3-b]pyridine-2-carboxamide (65 mg, 215.10 umol, 40.91% yield, 99.08% purity) was obtained as a white solid which was confirmed by LCMS and ¹H NMR.

LCMS (ESI) m/z 300.2 [M+H]⁺; 1H NMR (400 MHz, DMSO-d₆) δ=11.71 (s, 1H), 8.12 (br d, J=7.9 Hz, 1H), 7.76 (s, 1H), 7.01 (d, J=1.7 Hz, 1H), 4.07-3.98 (m, 1H), 2.48 (s, 3H), 2.31 (s, 3H), 1.82-1.65 (m, 6H), 1.63-1.45 (m, 8H).

### Example 66. MPL-092

### Synthesis of 4-fluoro-N-[(1S,2S,3S,5R)-2,6,6-trimethylnorpinan-3-yl]-1H-pyrrolo[2,3-c]pyridine-2-carboxamide

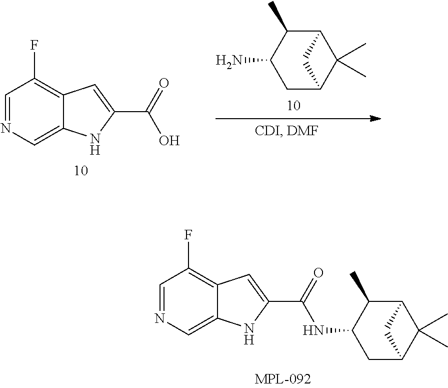 [SMILES: O=C(O)c1cc2c(F)cncc2[nH]1 ; C[C@@H]1[C@@H](N)C[C@H]2C[C@@H]1C2(C)C ; C[C@@H]1[C@@H](NC(=O)c2cc3c(F)cncc3[nH]2)C[C@H]2C[C@@H]1C2(C)C]

To a solution of 4-fluoro-1H-pyrrolo[2,3-c]pyridine-2-carboxylic acid (100 mg, 555.14 umol, 1 eq) in DMF (1 mL) was added CDI (117.02 mg, 721.68 umol, 1.3 eq). The mixture was stirred at 15° C. for 0.5 h. Then (1S,2S,3S,5R)-2,6,6-trimethylnorpinan-3-amine (110.61 mg, 721.68 umol, 1.3 eq) was added. The mixture was stirred at 15° C. for 12 hr. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. Then the crude product was purified by silica column chromatography (eluent of 20˜70% EtOAc/Petroleum ether gradient, 4 g silica column). All fractions found to contain product by TLC (Petroleum ether:EtOAc=1:1, R_{f}=0.3) were combined and evaporated. Compound 4-fluoro-N-[(1S,2S,3S,5R)-2,6,6-trimethylnorpinan-3-yl]-1H-pyrrolo[2,3-c]pyridine-2-carboxamide (30 mg, 94.36 umol, 17.00% yield, 99.20% purity) was obtained as a white solid.

LCMS (ESI) m/z 316.2 [M+H]⁺; ¹H NMR (400 MHz, DMSO-d₆) δ=12.47 (br s, 1H), 8.67-8.60 (m, 2H), 8.09 (s, 1H), 7.38 (s, 1H), 4.43-4.31 (m, 1H), 2.45-2.29 (m, 2H), 2.07 (br t, J=7.0 Hz, 1H), 1.94 (br s, 1H), 1.80 (br s, 1H), 1.70 (br dd, J=5.5, 12.9 Hz, 1H), 1.23-1.17 (m, 4H), 1.07-1.03 (m, 6H).

### Example 67. MPL-093

### Synthesis of N-(4,4-dimethylcyclohexyl)-4-fluoro-1H-pyrrolo[2,3-c]pyridine-2-carboxamide

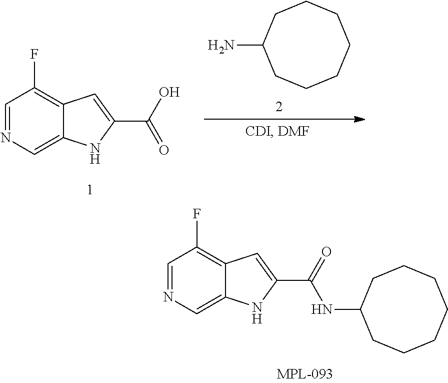 [SMILES: O=C(O)c1cc2c(F)cncc2[nH]1 ; NC1CCCCCCC1 ; O=C(NC1CCCCCCC1)c1cc2c(F)cncc2[nH]1]

To a solution of 4-fluoro-1H-pyrrolo[2,3-c]pyridine-2-carboxylic acid (100 mg, 555.14 umol, 1 eq) in DMF (2 mL) was added CDI (117.02 mg, 721.68 umol, 1.3 eq). The mixture was stirred at 30° C. for 0.5 h. Then cyclooctanamine (91.82 mg, 721.68 umol, 1.3 eq) was added. The mixture was stirred at 30° C. for 12 h. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. The residue was delivered without further purification. Compound N-cyclooctyl-4-fluoro-1H-pyrrolo[2,3-c]pyridine-2-carboxamide (70 mg, 238.22 umol, 42.91% yield, 98.47% purity) was obtained as a white solid.

LCMS (ESI) m/z 290.1 [M+H]⁺; 1H NMR (400 MHz, DMSO-d₆) δ=12.40 (br s, 1H), 8.62 (br s, 1H), 8.52 (br d, J=7.8 Hz, 1H), 8.05 (s, 1H), 7.34 (s, 1H), 7.38-7.28 (m, 1H), 7.38-7.28 (m, 1H), 7.38-7.28 (m, 1H), 7.38-7.28 (m, 1H), 7.38-7.28 (m, 1H), 4.03 (br s, 1H), 1.81-1.63 (m, 6H), 1.59-1.45 (m, 1H), 1.59-1.45 (m, 7H).

### Example 68. MPL-094

### N-(4,4-dimethylcyclohexyl)-4-fluoro-1H-pyrrolo[2,3-c]pyridine-2-carboxamide

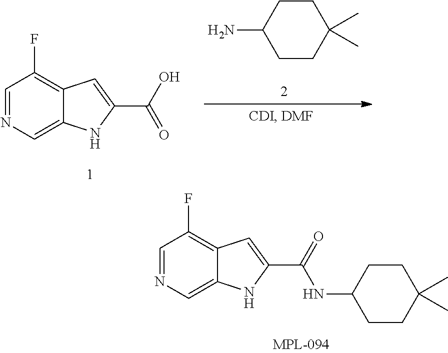

To a solution of 4-fluoro-1H-pyrrolo[2,3-c]pyridine-2-carboxylic acid (100 mg, 555.14 umol, 1 eq) in DMF (1 mL) was added CDI (108.02 mg, 666.16 umol, 1.2 eq). The mixture was stirred at 30° C. for 0.5 h. 4,4-dimethylcyclohexanamine (84.75 mg, 666.16 umol, 1.2 eq) was added. The mixture was stirred at 30° C. for 11.5 h. LCMS showed desired compound mass was detected. The reaction mixture was added to water (15 ml). Then it was filtered and the filter cake was washed with 10 mL of water, dried in vacuo to give crude product. The product N-(4,4-dimethylcyclohexyl)-4-fluoro-1H-pyrrolo[2,3-c]pyridine-2-carboxamide (36.5 mg, 119.84 umol, 21.59% yield, 95% purity) was obtained as white solid.

LCMS (ESI) m/z 290.2 [M+H]⁺; ¹H NMR (400 MHz, DMSO-d6) δ=12.44 (br s, 1H), 8.64 (d, J=2.2 Hz, 1H), 8.52 (br d, J=7.9 Hz, 1H), 8.08 (s, 1H), 7.33 (s, 1H), 3.75 (br s, 1H), 1.68 (br d, J=10.5 Hz, 2H), 1.61-1.48 (m, 2H), 1.45-1.37 (m, 2H), 1.30 (br d, J=12.3 Hz, 2H), 0.94 (d, J=8.8 Hz, 6H).

### Example 69. MPL-095

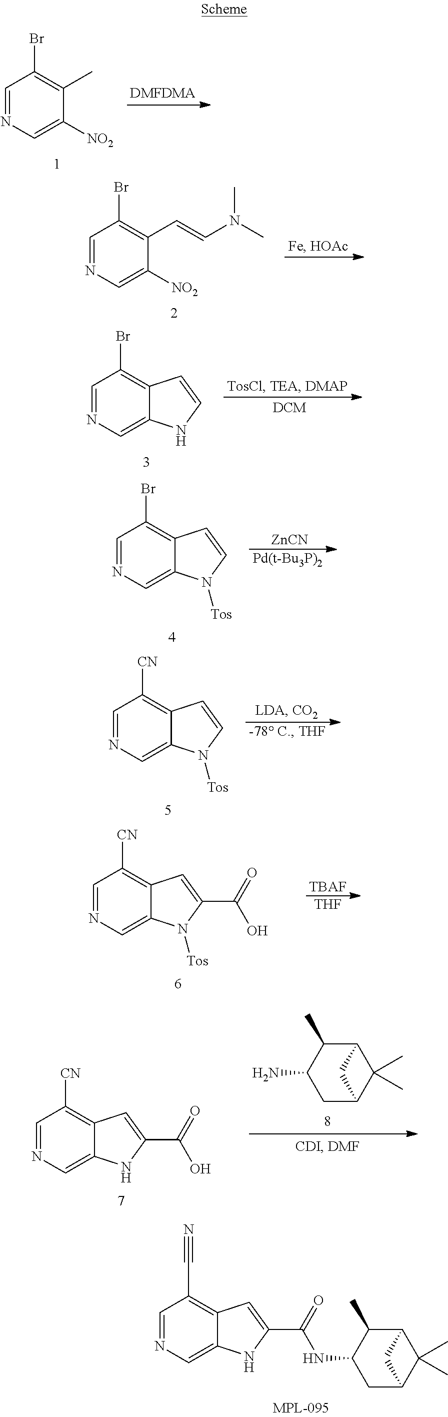

### Synthesis of 4-bromo-1-(p-tolylsulfonyl)pyrrolo[2,3-c]pyridine

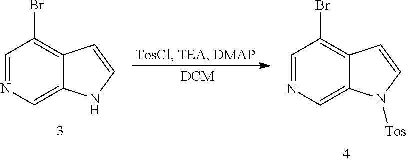

To a solution of 4-bromo-1H-pyrrolo[2,3-c]pyridine (3.11 g, 15.78 mmol, 1 eq) in DCM (50 mL) was added TosCl (3.91 g, 20.52 mmol, 1.3 eq), DMAP (192.83 mg, 1.58 mmol, 0.1 eq) and TEA (3.19 g, 31.57 mmol, 4.39 mL, 2 eq). The mixture was stirred at 15° C. for 12 hr. LCMS showed there were no starting material and main desired compound. The mixture was concentrated in reduced pressure. The crude product was purified by silica column chromatography (eluent of 0˜50% EtOAc/Petroleum ether gradient, 40 g silica column). All fractions found to contain product by TLC (Petroleum ether:EtOAc=3:1, Rf=0.3) were combined and evaporated. Compound 4-bromo-1-(p-tolylsulfonyl)pyrrolo[2,3-c]pyridine (5 g, 13.52 mmol, 85.68% yield, 95% purity) was obtained as a yellow solid. LCMS (ESI) m/z 352.4 [M+H]⁺

### Synthesis of 1-(p-tolylsulfonyl)pyrrolo[2,3-c]pyridine-4-carbonitrile

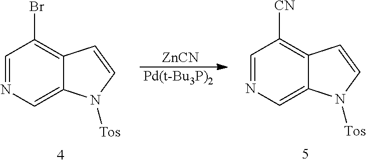

A mixture of 4-bromo-1-(p-tolylsulfonyl)pyrrolo[2,3-c]pyridine (4 g, 11.39 mmol, 1 eq), Zn(CN)₂ (2.67 g, 22.78 mmol, 1.45 mL, 2 eq), Pd(t-Bu₃P)₂ (582.04 mg, 1.14 mmol, 0.1 eq) in DMF (50 mL) was degassed and purged with N₂ for 3 times, and then the mixture was stirred at 110° C. for 12 hr under N₂ atmosphere. LCMS showed there were no starting material and main desired compound. The mixture was diluted with EtOAc (200 mL). It was washed with aqueous 5% LiCl (80 mL×3), dried with anhydrous Na₂SO₄, filtered and concentrated in vacuo. The crude product was purified by silica column chromatography (eluent of 0-50% EtOAc/Petroleum ether gradient, 80 g silica column). All fractions found to contain product by TLC (Petroleum ether:EtOAc=3:1, Rf=0.3) were combined and evaporated. Compound 1-(p-tolylsulfonyl)pyrrolo[2,3-c]pyridine-4-carbonitrile (690 mg, 2.09 mmol, 18.34% yield, 90% purity) was obtained as a white solid.

### Synthesis of 4-cyano-1-(p-tolylsulfonyl)pyrrolo[2,3-c]pyridine-2-carboxylic acid

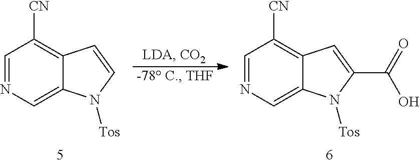

To a solution of 1-(p-tolylsulfonyl)pyrrolo[2,3-c]pyridine-4-carbonitrile (690 mg, 2.32 mmol, 1 eq) in THE (10 mL) was added LDA (2 M, 1.51 mL, 1.3 eq) at −78° C. under N₂ atmosphere. The mixture was stirred at −78° C. for 1 h. Then then the mixture was stirred at −78° C. under CO₂ (102.13 mg, 2.32 mmol, 1 eq) atmosphere for 0.5 h. LCMS showed there were no starting material and main desired compound. The reaction was quenched at −78° C. with saturated aqueous NH₄Cl (5 mL). There was much white precipitation which was filtered. The cake was dried under reduced pressure. The residue was used directly for next step without further purification. Compound 4-cyano-1-(p-tolylsulfonyl)pyrrolo[2,3-c]pyridine-2-carboxylic acid (1.0 g, crude) was obtained as a white solid. LCMS (ESI) m/z 342.0 [M+H]⁺

### Synthesis of 4-cyano-1H-pyrrolo[2,3-c]pyridine-2-carboxylic acid

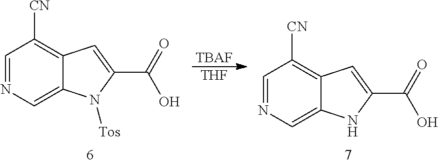

To a solution of 4-cyano-1-(p-tolylsulfonyl)pyrrolo[2,3-c]pyridine-2-carboxylic acid (1 g, 2.93 mmol, 1 eq) in THE (10 mL) was added TBAF (1 M, 20 mL, 6.83 eq). The mixture was stirred at 80° C. for 12 hr. LCMS showed there were no starting material and main desired compound. The mixture was concentrated in reduced pressure. The residue was diluted with H₂O (50 mL). The aqueous phase was extracted with EtOAc (15 mL×3). The combined organic phase was washed with saturated brine (10 mL×2). The residue was used directly for next step without further purification. Compound 4-cyano-1H-pyrrolo[2,3-c]pyridine-2-carboxylic acid (210 mg, 1.01 mmol, 34.47% yield, 90% purity) was obtained as a white solid. LCMS (ESI) m/z 188.0 [M+H]⁺

### Synthesis of 4-fluoro-N-[(1S,2S,3S,5R)-2,6,6-trimethylnorpinan-3-yl]-1H-pyrrolo[2,3-c]pyridine-2-carboxamide

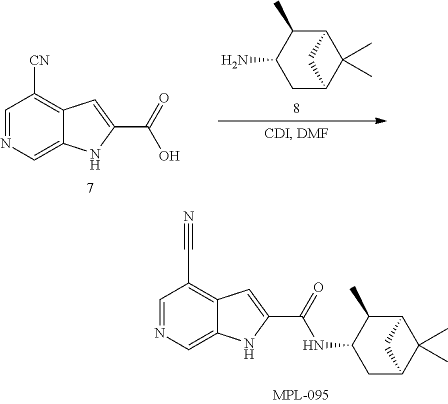 [SMILES: N#Cc1cncc2[nH]c(C(=O)O)cc12 ; C[C@@H]1[C@@H](N)C[C@H]2C[C@@H]1C2(C)C ; C[C@@H]1[C@@H](NC(=O)c2cc3c(C#N)cncc3[nH]2)C[C@H]2C[C@@H]1C2(C)C]

To a solution of 4-cyano-1H-pyrrolo[2,3-c]pyridine-2-carboxylic acid (60 mg, 320.59 umol, 1 eq) in DMF (1 mL) was added CDI (51.98 mg, 320.59 umol, 1 eq). The mixture was stirred at 30° C. for 0.5 h. (1S,2S,3S,5R)-2,6,6-trimethylnorpinan-3-amine (63.88 mg, 416.77 umol, 1.3 eq) was added and the mixture was stirred at 30° C. for 12 h. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. The residue was purified by prep-HPLC (column: YMC-Actus Triart C18 150*30 mm*5 um; mobile phase: [water (0.225% FA)-ACN]; B %: 45%-75%, 11 min). Compound 4-cyano-N-[(1S,2S,3S,5R)-2,6,6-trimethylnorpinan-3-yl]-1H-pyrrolo[2,3-c]pyridine-2-carboxamide (20 mg, 62.03 umol, 19.35% yield, 100% purity) was obtained as a white solid which was confirmed by LCMS and ¹H NMR.

LCMS (ESI) m/z 323.2 [M+H]⁺; ¹H NMR (400 MHz, DMSO-d₆) δ=12.78 (br s, 1H), 9.00 (s, 1H), 8.76 (br d, J=8.8 Hz, 1H), 8.64 (s, 1H), 7.53 (s, 1H), 7.54-7.51 (m, 1H), 4.38 (br d, J=9.2 Hz, 1H), 2.45-2.34 (m, 2H), 2.08 (br t, J=6.4 Hz, 1H), 1.94 (br s, 1H), 1.82 (br d, J=6.1 Hz, 1H), 1.74-1.67 (m, 1H), 1.22 (s, 3H), 1.19 (d, J=9.2 Hz, 1H), 1.07-1.04 (m, 6H).

### Example 70. MPL-096

### Synthesis of 4-cyano-N-cyclooctyl-1H-pyrrolo[2,3-c]pyridine-2-carboxamide

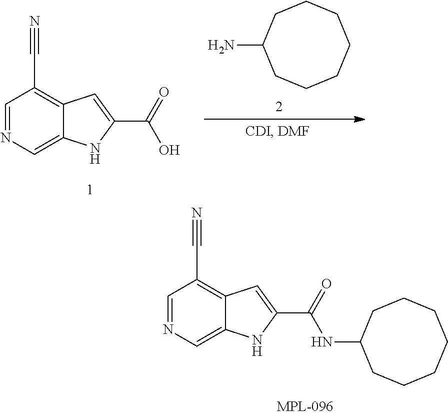 [SMILES: N#Cc1cncc2[nH]c(C(=O)O)cc12 ; NC1CCCCCCC1 ; N#Cc1cncc2[nH]c(C(=O)NC3CCCCCCC3)cc12]

To a solution of 4-cyano-1H-pyrrolo[2,3-c]pyridine-2-carboxylic acid (100 mg, 534.32 umol, 1 eq) in DMF (1 mL) was added CDI (112.63 mg, 694.61 umol, 1.3 eq). The mixture was stirred at 30° C. for 0.5 h. Cyclooctanamine (1.92 g, 15.09 mmol, 28.25 eq) was added and the reaction mixture was stirred at 30° C. for 12 h. LCMS showed there were no starting material and main desired compound. The reaction was added dropwise to H₂O (20 mL). There was much precipitation which was collected by filter. The cake was diluted with EtOAc (50 mL), dried with anhydrous MgSO₄, filtered. The filtrate was concentrated in vacuo. The crude product was purified by prep-HPLC (column: YMC-Actus Triart C18 150*30 mm*5 um; mobile phase: [water (0.225% FA)-ACN]; B %: 45%-72%, 11 min). Then it was purified by preparative TLC (Petroleum ether:EtOAc=1:1, Rf=0.3). Compound 4-cyano-N-cyclooctyl-1H-pyrrolo[2,3-c]pyridine-2-carboxamide (10 mg, 33.74 umol, 6.31% yield, 100% purity) was obtained as a white solid which was confirmed by LCMS and ¹H NMR.

LCMS (ESI) m/z 297.2 [M+H]⁺; 1H NMR (400 MHz, CDCl3-d) 10.71 (br s, 1H), 9.08 (s, 1H), 8.64 (s, 1H), 7.03 (s, 1H), 6.39 (br d, J=7.0 Hz, 1H), 4.34-4.21 (m, 1H), 2.05-1.96 (m, 2H), 1.75 (br d, J=9.2 Hz, 4H), 1.69-1.62 (m, 8H).

### Example 71. MPL-097

### Synthesis of 4-cyano-N-(4,4-dimethylcyclohexyl)-1H-pyrrolo[2,3-c]pyridine-2-carboxamide

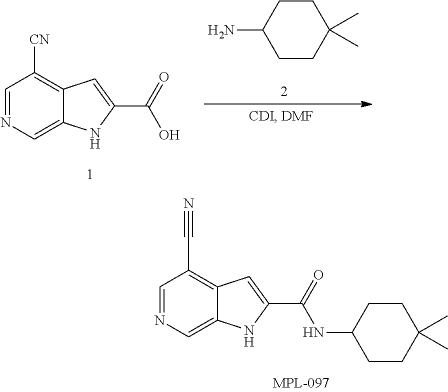

To a solution of 4-cyano-1H-pyrrolo[2,3-c]pyridine-2-carboxylic acid (60 mg, 320.59 umol, 1 eq) in DMF (1 mL) was added CDI (67.58 mg, 416.77 umol, 1.3 eq). The mixture was stirred at 30° C. for 0.5 h. 4,4-dimethylcyclohexanamine (53.02 mg, 416.77 umol, 1.3 eq) was added and the reaction mixture was stirred at 30° C. for 12 h. LCMS showed there were no starting material and main desired compound. The reaction was added dropwise to H₂O (20 mL). There was much precipitation which was collected by filter. The cake was diluted with EtOAc (50 mL), dried with anhydrous MgSO₄, filtered. The filtrate was concentrated in vacuo. The crude product was purified by prep-HPLC (column: YMC-Actus Triart C18 150*30 mm*5 um; mobile phase: [water (0.225% FA)-ACN]; B %: 42%-68%, 11 min) Compound 4-cyano-N-(4,4-dimethylcyclohexyl)-1H-pyrrolo[2,3-c]pyridine-2-carboxamide (15 mg, 49.44 umol, 15.42% yield, 97.69% purity) was obtained as a white solid which was confirmed by LCMS and 1H NMR.

LCMS (ESI) m/z 297.2 [M+H]⁺; 1H NMR (400 MHz, DMSO-d₆) δ=12.78 (br s, 1H), 9.00 (s, 1H), 8.70-8.62 (m, 2H), 7.48 (s, 1H), 7.51-7.45 (m, 1H), 7.51-7.45 (m, 1H), 3.83-3.69 (m, 1H), 3.83-3.69 (m, 1H), 1.68 (br d, J=9.6 Hz, 2H), 1.61-1.48 (m, 2H), 1.45-1.37 (m, 2H), 1.33-1.23 (m, 2H), 0.93 (d, J=9.2 Hz, 5H), 0.97-0.89 (m, 1H).

### Example 72. MPL-106

### Synthesis of 4-fluoro-N-(1,7,7-trimethylnorbornan-2-yl)-1H-pyrrolo[2,3-b]pyridine-2-carboxamide

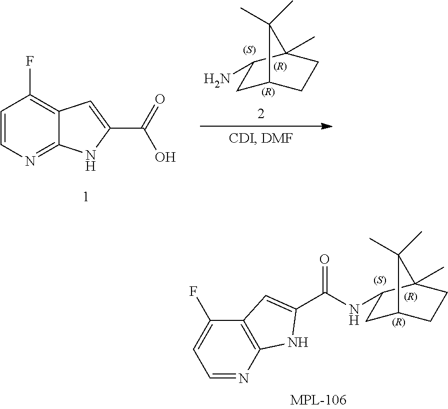

To a solution of 4-fluoro-1H-pyrrolo[2,3-b]pyridine-2-carboxylic acid (100 mg, 555.14 umol, 1 eq) in DMF (2 mL) was added CDI (108.02 mg, 666.16 umol, 1.2 eq). The mixture was stirred at 30° C. for 0.5 h. Then 1,7,7-trimethylnorbornan-2-amine (102.10 mg, 666.16 umol, 1.2 eq) was added. The mixture was stirred at 30° C. for another 0.5 h. LC-MS showed the starting material 3 was consumed completely and one main peak with desired mass was detected. The mixture was concentrated under reduced pressure to remove the DCM, and added to water (20 mL), filtered. The filter cake was washed with 10 mL of water, dried under reduced pressure to give the product. The residue was purified by column chromatography (SiO₂, DCM:MeOH=1:0 to 200:1). The product 4-fluoro-N-(1,7,7-trimethylnorbornan-2-yl)-1H-pyrrolo[2,3-b]pyridine-2-carboxamide (112.3 mg, 356.07 umol, 64.14% yield, 100% purity) was obtained as white solid.

LCMS (ESI) m/z 316.2 [M+H]⁺; ¹H NMR (400 MHz, DMSO-δ₆)=12.50 (br s, 1H), 8.33 (dd, J=5.5, 8.2 Hz, 1H), 8.12 (br d, J=8.5 Hz, 1H), 7.37 (d, J=1.8 Hz, 1H), 7.02 (dd, J=5.3, 10.4 Hz, 1H), 4.38 (br s, 1H), 2.20 (br s, 1H), 1.82-1.64 (m, 3H), 1.40 (br d, J=10.1 Hz, 1H), 1.27 (br s, 1H), 1.14 (dd, J=4.7, 13.0 Hz, 1H), 0.96 (s, 3H), 0.87 (s, 3H), 0.78 (s, 3H).

### Example 73. MPL-107

### Synthesis of 4-cyano-N-(1,7,7-trimethylnorbornan-2-yl)-1H-pyrrolo[2,3-b]pyridine-2-carboxamide

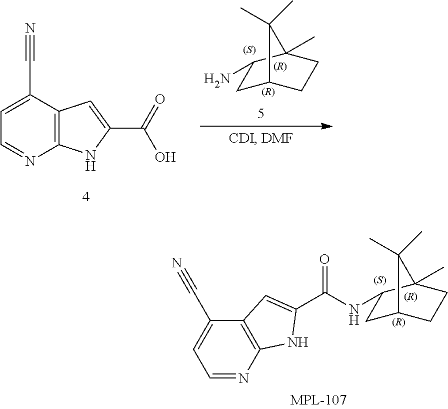

To a solution of 4-cyano-1H-pyrrolo[2,3-b]pyridine-2-carboxylic acid (110 mg, 587.75 umol, 1 eq) in DMF (2 mL) was added CDI (114.36 mg, 705.30 umol, 1.2 eq), the mixture was stirred at 30° C. for 0.5 h, then 1,7,7-trimethylnorbornan-2-amine (108.10 mg, 705.30 umol, 1.2 eq) was added. The mixture was stirred at 30° C. for another 0.5 h. LC-MS showed the starting material 4 was consumed completely and one main peak with desired mass was detected. The mixture was added to water (20 mL), filtered. The filter cake was washed with 10 mL of water, dried under reduced pressure to give 4-cyano-N-(1,7,7-trimethylnorbornan-2-yl)-1H-pyrrolo[2,3-b]pyridine-2-carboxamide (129.2 mg, 400.74 umol, 68.18% yield, 100% purity) was obtained as white solid.

LCMS (ESI) m/z 323.2 [M+H]⁺; ¹H NMR (400 MHz, DMSO-δ₆)=12.80 (br s, 1H), 8.52 (d, J=4.7 Hz, 1H), 8.32 (br d, J=8.2 Hz, 1H), 7.64 (d, J=4.7 Hz, 1H), 7.57 (s, 1H), 4.40 (br s, 1H), 2.26-2.17 (m, 1H), 1.82-1.64 (m, 3H), 1.45-1.37 (m, 1H), 1.28 (br t, J=11.8 Hz, 1H), 1.17 (dd, J=4.6, 12.8 Hz, 1H), 0.97 (s, 3H), 0.87 (s, 3H), 0.78 (s, 3H).

### Example 74. MPL-109

### Synthesis of N-(1,1-dimethylsilinan-4-yl)-4-fluoro-1H-pyrrolo[2,3-b]pyridine-2-carboxamide

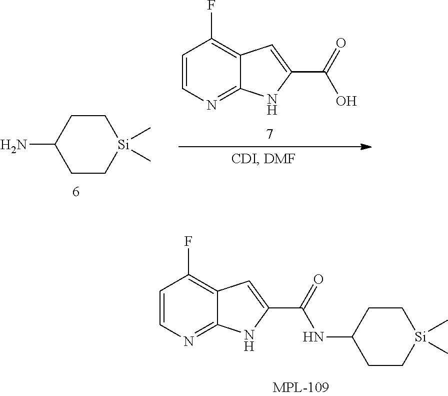

To a solution of 4-fluoro-1H-pyrrolo[2,3-b]pyridine-2-carboxylic acid (50 mg, 277.57 umol, 1 eq) in DMF (2 mL) was added CDI (67.51 mg, 416.35 umol, 1.5 eq), the mixture was stirred at 30° C. for 0.5 h, then 1,1-dimethylsilinan-4-amine (59.66 mg, 416.35 umol, 1.5 eq) was added and the mixture was stirred at 30° C. for another 0.5 h. LC-MS showed the starting material 6 was consumed completely and one main peak with desired mass was detected. The mixture was added to water (20 mL), and stirred for 10 min, filtered. The filter cake was dried under reduced pressure to give the crude product. The product N-(1,1-dimethylsilinan-4-yl)-4-fluoro-1H-pyrrolo[2,3-b]pyridine-2-carboxamide (40.3 mg, 122.81 umol, 44.24% yield, 93.072% purity) was obtained as white solid.

LCMS (ESI) m/z 306.1 [M+H]⁺; ¹H NMR (400 MHz, DMSO-δ₆)=12.43 (br s, 1H), 8.32 (dd, J=5.4, 8.3 Hz, 2H), 7.22 (s, 1H), 7.00 (dd, J=5.4, 10.3 Hz, 1H), 3.78-3.66 (m, 1H), 1.99 (br s, 2H), 1.59 (br d, J=13.4 Hz, 2H), 0.78 (br d, J=14.4 Hz, 2H), 0.66-0.55 (m, 2H), 0.12-0.01 (m, 6H).

### Example 75. MPL-110

### Synthesis of 4-cyano-N-(1,1-dimethylsilinan-4-yl)-1H-pyrrolo[2,3-b]pyridine-2-carboxamide

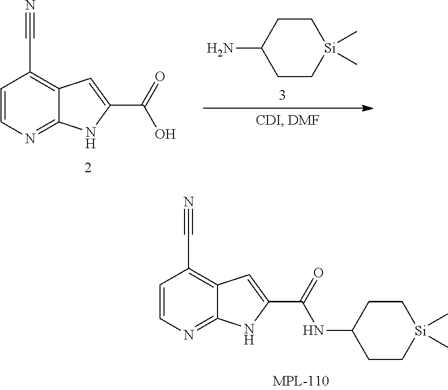

To a solution of 4-cyano-1H-pyrrolo[2,3-b]pyridine-2-carboxylic acid (600.00 mg, 3.21 mmol, 9.69e-1 eq) in DMF (6 mL) was added CDI (804.70 mg, 4.96 mmol, 1.5 eq), the mixture was stirred at 25° C. for 0.5 h, then 1,1-dimethylsilinan-4-amine (711.16 mg, 4.96 mmol, 1.5 eq) was added. The mixture was stirred at 25° C. for 0.5 h. LCMS (in MeOH) showed the reaction was consumed. The mixture was dropwise added to water (50 mL), and stirred for 10 min, filtered. The filter cake was dried under reduced pressure to give the crude product. The product 4-cyano-N-(1,1-dimethylsilinan-4-yl)-1H-pyrrolo[2,3-b]pyridine-2-carboxamide (705.1 mg, 2.19 mmol, 66.18% yield, 97.026% purity) was obtained as white solid.

LCMS (ESI) m/z 313.2 [M+H]⁺; ¹H NMR (500 MHz, DMSO-d₆) δ=12.84 (br s, 1H), 8.51 (br d, J=4.9 Hz, 2H), 7.63 (d, J=4.6 Hz, 1H), 7.41 (s, 1H), 3.73 (br d, J=8.1 Hz, 1H), 2.01 (br d, J=9.8 Hz, 2H), 1.68-1.54 (m, 2H), 0.84-0.75 (m, 2H), 0.62 (dt, J=4.5, 14.1 Hz, 2H), 0.12-0.01 (m, 6H).

### Example 76. MPL-111

### Synthesis of N-(1,1-dimethylsilinan-4-yl)-4-(trifluoromethyl)-1H-pyrrolo[2,3-b]pyridine-2-carboxamide

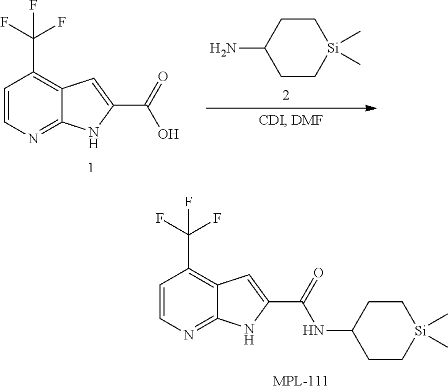

To a solution of 4-(trifluoromethyl)-1H-pyrrolo[2,3-b]pyridine-2-carboxylic acid (0.2 g, 869.02 umol, 1 eq) in DMF (5 mL, dried by CaH₂) was added CDI (183.18 mg, 1.13 mmol, 1.3 eq). The mixture was stirred at 15° C. for 0.5 hr. Then 1,1-dimethylsilinan-4-amine (161.89 mg, 1.13 mmol, 1.3 eq) was added. The mixture was stirred at 15° C. further 1 hr. LC-MS showed reactant was consumed completely and desired mass was detected. The reaction mixture was mixed into water (50 mL). Filtered, the filtered cake was washed with water (10 mL*2) Compound N-(1,1-dimethylsilinan-4-yl)-4-(trifluoromethyl)-1H-pyrrolo[2,3-b]pyridine-2-carboxamide (258.7 mg, 705.96 umol, 81.24% yield, 96.992% purity) was obtained as a white solid.

LCMS (ESI) m/z 355.13 [M+H]⁺; ¹H NMR (500 MHz, DMSO-d₆) δ=12.59 (br s, 1H) 8.45 (d, J=4.73 Hz, 1H) 8.41 (d, J=8.24 Hz, 1H) 7.38 (d, J=4.88 Hz, 1H) 7.24 (d, J=1.53 Hz, 1H) 3.59-3.67 (m, 1H) 1.92 (br dd, J=9.23, 3.59 Hz, 2H) 1.46-1.56 (m, 2H) 0.69 (br d, J=14.50 Hz, 2H) 0.53 (td, J=14.19, 4.73 Hz, 2H) 0.00 (s, 3H) −0.07-−0.03 (m, 3H).

### Example 77. MPL-118

### Synthesis of N-cyclooctyl-4-fluoro-6-methyl-1H-pyrrolo[2,3-b]pyridine-2-carboxamide

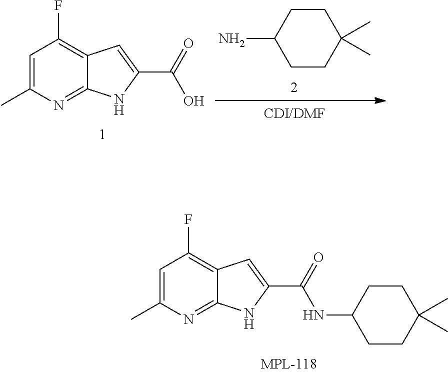

To a solution of 4-fluoro-6-methyl-1H-pyrrolo[2,3-b]pyridine-2-carboxylic acid (60 mg, 309.02 umol, 1 eq) in DMF (1.5 mL) was added CDI (60.13 mg, 370.82 umol, 1.2 eq), the mixture was stirred at 30° C. for 0.5 h, then 4,4-dimethylcyclohexanamine (47.18 mg, 370.82 umol, 1.2 eq) was added, the mixture was stirred at 30° C. for another 0.5 h. LC-MS showed the starting material 1 was consumed completely and one main peak with desired mass was detected. The mixture was added to water (20 mL), filtered and the filter cake was washed with 10 mL×3 of petroleum ether, dried under reduced pressure to give the crude product. The crude product was purified by prep-HPLC (column: YMC-Actus Triart C18 150*30 mm*5 um; mobile phase: [water (0.225% FA)-ACN]; B %: 50%-80%, 11 min). The product N-(4,4-dimethylcyclohexyl)-4-fluoro-6-methyl-1H-pyrrolo[2,3-b]pyridine-2-carboxamide (23.6 mg, 76.58 umol, 24.78% yield, 98.445% purity) was obtained as white solid.

LCMS (ESI) m/z 304.1 [M+H]⁺; ¹H NMR (400 MHz, DMSO-δ₆)=12.23 (br s, 1H), 8.24 (d, J=7.9 Hz, 1H), 7.17 (d, J=2.2 Hz, 1H), 6.89 (d, J=11.4 Hz, 1H), 3.72 (br d, J=7.9 Hz, 1H), 2.53 (s, 3H), 1.67 (br d, J=9.6 Hz, 2H), 1.51 (br d, J=12.3 Hz, 2H), 1.45-1.38 (m, 2H), 1.32-1.23 (m, 2H), 0.93 (d, J=7.9 Hz, 6H).

### Example 78. MPL-120

### Synthesis of 5-methoxy-N-[(1S,2S,3S,5R)-2,6,6-trimethylnorpinan-3-yl]-1H-pyrrolo[2,3-c]pyridine-2-carboxamide

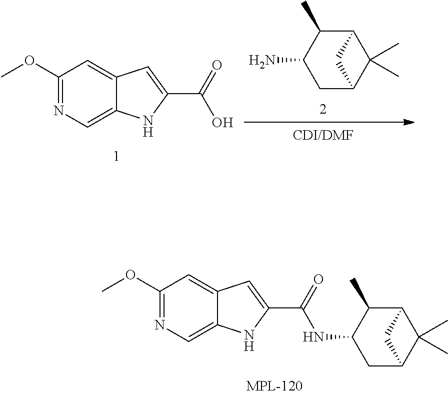

To a solution of 5-methoxy-1H-pyrrolo[2,3-c]pyridine-2-carboxylic acid (100 mg, 520.37 umol, 1 eq) in DMF (1 mL) was added CDI (109.69 mg, 676.48 umol, 1.3 eq). The mixture was stirred at 30° C. for 0.5 hr. Then (1S,2S,3S,5R)-2,6,6-trimethylnorpinan-3-amine (103.68 mg, 676.48 umol, 1.3 eq) was added. The mixture was stirred at 30° C. for 12 hr. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. The residue was purified by prep-HPLC (column: YMC-Actus Triart C18 150*30 mm*5 um; mobile phase: [water (0.225% FA)-ACN]; B %: 34%-64%, 11 min). Compound 5-methoxy-N-[(1S,2S,3S,5R)-2,6,6-trimethylnorpinan-3-yl]-1H-pyrrolo[2,3-c]pyridine-2-carboxamide (40 mg, 117.87 umol, 22.65% yield, 96.48% purity) was obtained as a white solid.

LCMS (ESI) m/z 328.2 [M+H]⁺; 1H NMR (400 MHz, DMSO-d₆) δ=11.97 (br s, 1H), 8.61 (br d, J=8.3 Hz, 1H), 8.42 (s, 1H), 7.13 (s, 1H), 7.11-7.07 (m, 1H), 7.09 (s, 1H), 4.36 (quin, J=8.1 Hz, 1H), 3.87 (s, 3H), 2.39-2.32 (m, 1H), 2.41 (br s, 1H), 2.11-2.02 (m, 1H), 1.92 (br s, 1H), 1.79 (br t, J=5.4 Hz, 1H), 1.73-1.64 (m, 1H), 1.68 (br dd, J=6.5, 11.9 Hz, 1H), 1.24-1.16 (m, 4H), 1.07-1.01 (m, 6H).

### Example 79. MPL-121

### Synthesis of N-(4,4-dimethylcyclohexyl)-5-methoxy-1H-pyrrolo[2,3-c]pyridine-2-carboxamide

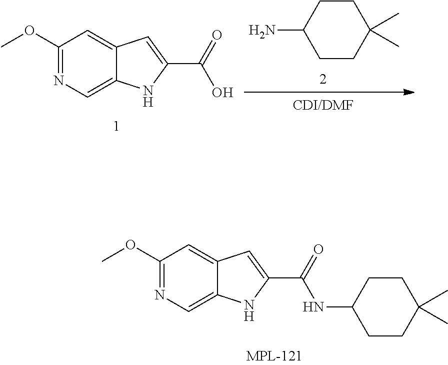

To a solution of 5-methoxy-1H-pyrrolo[2,3-c]pyridine-2-carboxylic acid (100 mg, 520.37 umol, 1 eq) in DMF (1 mL) was added CDI (109.69 mg, 676.48 umol, 1.3 eq). The mixture was stirred at 30° C. for 0.5. Then 4,4-dimethylcyclohexanamine (86.07 mg, 676.48 umol, 1.3 eq) was added. The mixture was stirred at 30° C. for 12 hr. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. The residue was delivered without further purification. Compound N-(4,4-dimethylcyclohexyl)-5-methoxy-1H-pyrrolo[2,3-c]pyridine-2-carboxamide (80 mg, 253.71 umol, 48.76% yield, 95.58% purity) was obtained as a yellow solid which was confirmed by LCMS and ¹H NMR.

LCMS (ESI) m/z 302.2 [M+H]⁺; 1H NMR (400 MHz, DMSO-d₆) δ=11.71 (br s, 1H), 8.46-8.29 (m, 2H), 7.01 (s, 1H), 6.91 (s, 1H), 3.81 (s, 3H), 3.72 (br d, J=8.8 Hz, 1H), 3.76-3.66 (m, 1H), 1.64 (br d, J=9.2 Hz, 2H), 1.58-1.46 (m, 2H), 1.42-1.35 (m, 2H), 1.30-1.22 (m, 2H), 0.93 (s, 3H), 0.91 (s, 3H).

### Example 80. MPL-122

### Synthesis of 7-fluoro-5-methyl-N-[(1S,2S,3S,5R)-2,6,6-trimethylnorpinan-3-yl]-1H-pyrrolo[2,3-c]pyridine-2-carboxamide

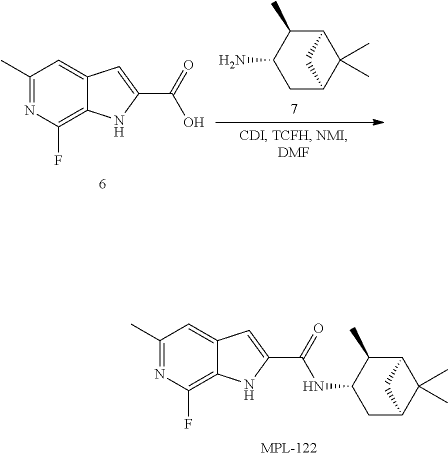

To a solution of 7-fluoro-5-methyl-1H-pyrrolo[2,3-c]pyridine-2-carboxylic acid (50 mg, 206.01 umol, 1 eq) in DMF (1 mL) was added CDI (43.43 mg, 267.82 umol, 1.3 eq). The mixture was stirred at 30° C. for 0.5 h. (1S,2S,3S,5R)-2,6,6-trimethylnorpinan-3-amine (37.89 mg, 247.22 umol, 1.2 eq) was added and the mixture was stirred at 30° C. for 12 h. LCMS showed there were starting material and main desired compound. (1S,2S,3S,5R)-2,6,6-trimethylnorpinan-3-amine (16 mg, 0.6 eq) was added and the mixture was stirred at 30° C. for another 12 hr. LCMS showed there were starting material and main desired compound. [chloro(dimethylamino)methylene]-dimethyl-ammonium; hexafluorophosphate (75.14 mg, 267.82 umol, 1.3 eq) and 1-methylimidazole (50.74 mg, 618.04 umol, 49.27 uL, 3 eq) was added and the mixture was stirred at 30° C. for 12 h. LCMS showed there were no starting material and main desired compound. The reaction was added dropwise to H₂O (20 mL). There was much precipitation which was collected by filter. The cake was diluted in EtOAc (20 mL), dried with anhydrous MgSO₄, filtered. The filtrate was concentrated in vacuo. The residue was purified by prep. TLC (SiO₂, Petroleum ether:EtOAc=2:1, Rf=0.3). Compound 7-fluoro-5-methyl-N-[(1S,2S,3S,5R)-2,6,6-trimethylnorpinan-3-yl]-1H-pyrrolo[2,3-c]pyridine-2-carboxamide (15 mg, 45.44 umol, 22.05% yield, 99.78% purity) was obtained as a white solid.

LCMS (ESI) m/z 330.2 [M+H]⁺; ¹H NMR (500 MHz, CHLOROFORM-d) 10.08 (br s, 1H), 7.20 (d, J=2.7 Hz, 1H), 6.77 (s, 1H), 6.13 (br d, J=8.5 Hz, 1H), 4.59-4.52 (m, 1H), 2.79-2.69 (m, 1H), 2.53 (s, 3H), 2.51-2.45 (m, 1H), 2.05-2.00 (m, 1H), 1.96-1.87 (m, 2H), 1.68 (br dd, J=2.1, 6.2 Hz, 1H), 1.26 (s, 3H), 1.19 (d, J=7.0 Hz, 3H), 1.11 (s, 3H), 0.93 (d, J=10.1 Hz, 1H).

### Example 81. MPL-124

### Synthesis of 5-methyl-N-[(1S,2S,3S,5R)-2,6,6-trimethylnorpinan-3-yl]-1H-pyrrolo[2,3-c]pyridine-2-carboxamide

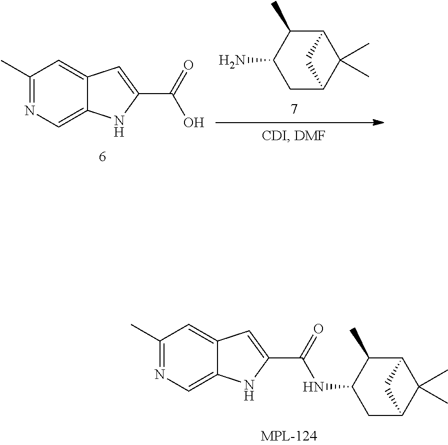

To a solution of 5-methyl-1H-pyrrolo[2,3-c]pyridine-2-carboxylic acid (120 mg, 272.46 umol, 1 eq) in DMF (1 mL) was added CDI (66.27 mg, 408.69 umol, 1.5 eq). The mixture was stirred at 30° C. for 0.5 h. (1S,2S,3S,5R)-2,6,6-trimethylnorpinan-3-amine (62.64 mg, 408.69 umol, 1.5 eq) was added and the mixture was stirred at 30° C. for 12 h. LCMS showed there were no starting material and main desired compound. The reaction was added dropwise to H₂O (20 mL). There was much precipitation which was collected by filter. The cake was diluted in EtOAc (10 mL), dried with anhydrous MgSO₄, filtered. The filtrate was concentrated in vacuo. The crude product was purified by silica column chromatography (eluent of 50˜100% EtOAc/Petroleum ether gradient, 4 g silica column). All fractions found to contain product by TLC (Petroleum ether:EtOAc=0:1, Rf=0.3) were combined and evaporated. Compound 5-methyl-N-[(1S,2S,3S,5R)-2,6,6-trimethylnorpinan-3-yl]-1H-pyrrolo[2,3-c]pyridine-2-carboxamide (30 mg, 94.91 umol, 34.83% yield, 98.52% purity) was obtained as a white solid.

LCMS (ESI) m/z 312.2 [M+H]⁺; ¹H NMR (500 MHz, DMSO-d6) δ=11.88 (br s, 1H), 8.67 (s, 1H), 8.55 (br d, J=8.5 Hz, 1H), 7.43 (s, 1H), 7.13 (s, 1H), 4.39 (quin, J=8.1 Hz, 1H), 2.47-2.41 (m, 1H), 2.37 (br d, J=6.7 Hz, 1H), 2.08 (br t, J=7.1 Hz, 1H), 1.95 (br s, 1H), 1.81 (br t, J=5.2 Hz, 1H), 1.71 (br dd, J=6.0, 12.8 Hz, 1H), 1.25-1.19 (m, 4H), 1.08-1.04 (m, 6H).

### Example 82. MPL-125

### Synthesis of N-(4,4-dimethylcyclohexyl)-5-methyl-1H-pyrrolo[2,3-c]pyridine-2-carboxamide

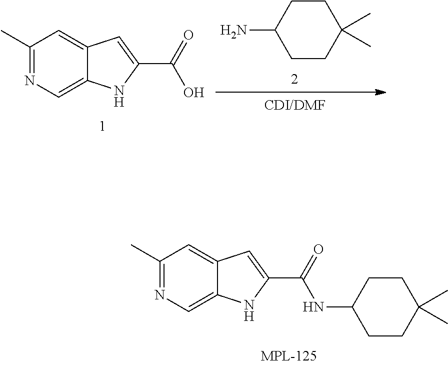

To a solution of 5-methyl-1H-pyrrolo[2,3-c]pyridine-2-carboxylic acid (200 mg, 454.10 umol, 1 eq) in DMF (1 mL) was added CDI (95.72 mg, 590.33 umol, 1.3 eq). The mixture was stirred at 30° C. for 0.5 h. 4,4-dimethylcyclohexanamine (75.11 mg, 590.33 umol, 1.3 eq) was added and the mixture was stirred at 30° C. for 12 h. LCMS showed there were no starting material and main desired compound. The reaction was added dropwise to H₂O (20 mL). There was much precipitation which was collected by filter. The cake was diluted in EtOAc (5 mL), dried with anhydrous MgSO₄, filtered. The filtrate was concentrated in vacuo. The crude product was purified by silica column chromatography (eluent of 50˜100% EtOAc/Petroleum ether gradient, 12 g silica column). All fractions found to contain product by TLC (Petroleum ether:EtOAc=0:1, Rf=0.2) were combined and evaporated. Compound N-(4,4-dimethylcyclohexyl)-5-methyl-1H-pyrrolo[2,3-c]pyridine-2-carboxamide (15 mg, 50.31 umol, 11.08% yield, 95.72% purity) was obtained as a white solid.

LCMS (ESI) m/z 286.2 [M+H]⁺; ¹H NMR (500 MHz, DMSO-d₆) δ=11.86 (br s, 1H), 8.64 (s, 1H), 8.43 (br d, J=8.1 Hz, 1H), 7.41 (s, 1H), 7.07 (s, 1H), 3.74-3.70 (m, 1H), 2.48 (br s, 3H), 1.65 (br d, J=10.7 Hz, 2H), 1.57-1.47 (m, 2H), 1.39 (br d, J=12.7 Hz, 2H), 1.30-1.21 (m, 2H), 0.92 (br d, J=10.7 Hz, 6H).

### Example 83. MPL-128

### Synthesis of 5-fluoro-N-[(1S,2S,3S,5R)-2,6,6-trimethylnorpinan-3-yl]-1H-pyrrolo[2,3-c]pyridine-2-carboxamide

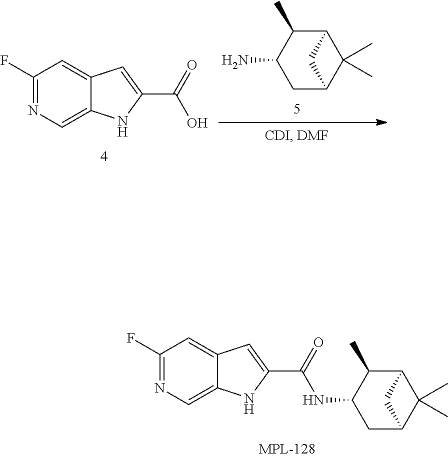 [SMILES: O=C(O)c1cc2cc(F)ncc2[nH]1 ; C[C@@H]1[C@@H](N)C[C@H]2C[C@@H]1C2(C)C ; C[C@@H]1[C@@H](NC(=O)c2cc3cc(F)ncc3[nH]2)C[C@H]2C[C@@H]1C2(C)C]

To a solution of 5-fluoro-1H-pyrrolo[2,3-c]pyridine-2-carboxylic acid (70 mg, 388.60 umol, 1 eq) in DMF (1 mL) was added CDI (81.91 mg, 505.17 umol, 1.3 eq). The mixture was stirred at 30° C. for 0.5 h. Then (1S,2S,3S,5R)-2,6,6-trimethylnorpinan-3-amine (77.42 mg, 505.17 umol, 1.3 eq) was added. The reaction mixture was stirred at 30° C. for 12 h. LCMS showed there were main starting material and desired compound. (1S,2S,3S,5R)-2,6,6-trimethylnorpinan-3-amine (38.5 mg, 0.5 eq) was added. The mixture was stirred at 30° C. for another 12 h. LCMS showed there was no starting material and main desired compound. The reaction was added dropwise to H₂O (20 mL). There was much precipitation which was collected by filter. The cake was diluted in EtOAc (20 mL) and concentrated in reduced pressure. The crude product was purified by silica column chromatography (eluent of 0˜40% EtOAc/Petroleum ether gradient, 4 g silica column). All fractions found to contain product by TLC (Petroleum ether:EtOAc=3:1, Rf=0.3) were combined and evaporated. Compound 5-fluoro-N-[(1S,2S,3S,5R)-2,6,6-trimethylnorpinan-3-yl]-1H-pyrrolo[2,3-c]pyridine-2-carboxamide (50 mg, 158.24 umol, 40.72% yield, 99.81% purity) was obtained as a white solid which was confirmed by LCMS and ¹H NMR.

LCMS (ESI) m/z 316.2 [M+H]⁺; ¹H NMR (400 MHz, DMSO-d₆) δ=12.13 (s, 1H), 8.63 (d, J=8.7 Hz, 1H), 8.38 (s, 1H), 7.32 (s, 1H), 7.24 (s, 1H), 4.38 (quin, J=8.1 Hz, 1H), 2.44 (br t, J=11.9 Hz, 1H), 2.37 (q, J=6.7 Hz, 1H), 2.08 (quin, J=7.2 Hz, 1H), 1.95 (br s, 1H), 1.81 (t, J=5.8 Hz, 1H), 1.71 (br dd, J=6.5, 12.7 Hz, 1H), 1.23 (s, 3H), 1.21 (d, J=9.6 Hz, 1H), 1.08-1.04 (m, 6H).

### Example 84. MPL-129

### Synthesis of N-(4,4-dimethylcyclohexyl)-5-fluoro-1H-pyrrolo[2,3-c]pyridine-2-carboxamide

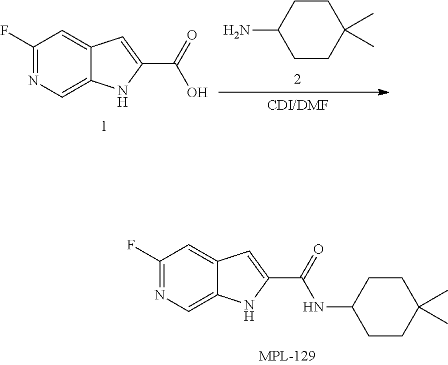

To a solution of 5-fluoro-1H-pyrrolo[2,3-c]pyridine-2-carboxylic acid (60 mg, 333.08 umol, 1 eq) in DMF (1 mL) was added CDI (70.21 mg, 433.01 umol, 1.3 eq). The mixture was stirred at 30° C. for 0.5 h. Then 4,4-dimethylcyclohexanamine (55.09 mg, 433.01 umol, 1.3 eq) was added. The reaction mixture was stirred at 30° C. for 12 h. LCMS showed there were main starting material and desired compound. 4,4-dimethylcyclohexanamine (28 mg, 0.5 eq) was added. The mixture was stirred at 30° C. for another 12 h. LCMS showed no starting material but one major product. The reaction was added dropwise to H₂O (20 mL). There was much precipitation which was collected by filter. The cake was diluted in CH₃CN (5 mL) and H₂O (20 mL), then lyophilized. The crude product was purified by silica column chromatography (eluent of 0˜50% EtOAc/Petroleum ether gradient, 4 g silica column). All fractions found to contain product by TLC (Petroleum ether:EtOAc=3:1, Rf=0.3) were combined and evaporated. Then the crude product was purified by prep-HPLC (column: YMC-Actus Triart C18 50*30 mm*5 um; mobile phase: [water (0.225% FA)-ACN]; B %: 47%-75%, 11 min). Compound N-(4,4-dimethylcyclohexyl)-5-fluoro-1H-pyrrolo[2,3-c]pyridine-2-carboxamide (50 mg, 172.80 umol, 51.88% yield, 100% purity) was obtained as a white solid which was confirmed by LCMS and ¹H NMR.

LCMS (ESI) m/z 290.1 [M+H]⁺; 1H NMR (500 MHz, CHLOROFORM-d) 10.39 (br s, 1H), 8.51 (s, 1H), 7.11 (s, 1H), 6.80 (s, 1H), 6.21 (br d, J=7.8 Hz, 1H), 4.03-3.95 (m, 1H), 1.97-1.91 (m, 2H), 1.56-1.46 (m, 4H), 1.44-1.36 (m, 2H), 0.98 (s, 6H).

### Example 85. MPL-130

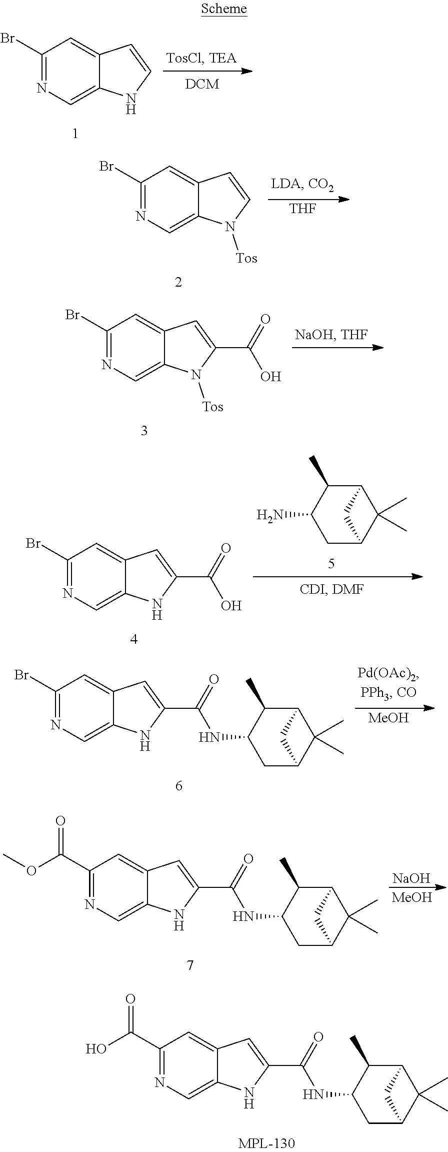

### Synthesis of 5-bromo-N-[(1S,2S,3S,5R)-2,6,6-trimethylnorpinan-3-yl]-3aH-pyrrolo[2,3-c]pyridine-2-carboxamide

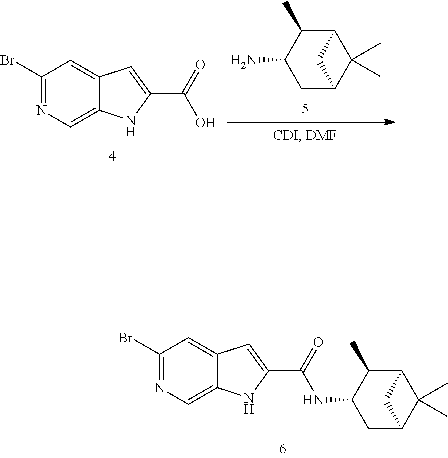 [SMILES: O=C(O)c1cc2cc(Br)ncc2[nH]1 ; C[C@@H]1[C@@H](N)C[C@H]2C[C@@H]1C2(C)C ; C[C@@H]1[C@@H](NC(=O)c2cc3cc(Br)ncc3[nH]2)C[C@H]2C[C@@H]1C2(C)C]

To a solution of 5-bromo-3aH-pyrrolo[2,3-c]pyridine-2-carboxylic acid (500 mg, 2.07 mmol, 1 eq) in DMF (5 mL) was added CDI (504.53 mg, 3.11 mmol, 1.5 eq), the mixture was stirred at 30° C. for 0.5 h, then (1S,2S,3S,5R)-2,6,6-trimethylnorpinan-3-amine (476.88 mg, 3.11 mmol, 1.5 eq) was added and the mixture was stirred for another 0.5 h at the same temperature. LCMS showed the desired mass was detected. The mixture was dropwise added to water (50 mL), and stirred for 10 min, filtered and the filter cake was dried under reduced pressure to give the crude product. The product 5-bromo-N-[(1S,2S,3S,5R)-2,6,6-trimethylnorpinan-3-yl]-3aH-pyrrolo[2,3-c]pyridine-2-carboxamide (750 mg, 1.79 mmol, 86.48% yield, 90% purity) was obtained as white solid.

### Synthesis of methyl 2-[[(1S,2S,3S,5R)-2,6,6-trimethylnorpinan-3-yl]carbamoyl]-1H-pyrrolo[2,3-c]pyridine-5-carboxylae

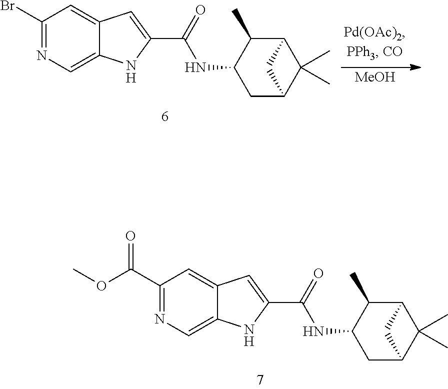

To a solution of 5-bromo-N-[(1S,2S,3S,5R)-2,6,6-trimethylnorpinan-3-yl]-1H-pyrrolo[2,3-c]pyridine-2-carboxamide (450 mg, 1.20 mmol, 1 eq) in DMF (2 mL) was added MeOH (2 mL), Pd(OAc)₂ (26.85 mg, 119.59 umol, 0.1 eq), PPh₃ (62.73 mg, 239.18 umol, 0.2 eq) and TEA (605.06 mg, 5.98 mmol, 832.27 uL, 5 eq). The mixture was evacuated 3 times with CO and stirred at 80° C. for 108 hr under carbon monoxide in 3 atm. LC-MS showed one peak with desired mass was detected and the reactant 6 was consumed completely. The mixture was filtered and the filter was concentrated under reduced pressure to give a residue. The residue was used directly for the next step without purification. The crude product methyl 2-[[(1S,2S,3S,5R)-2,6,6-trimethylnorpinan-3-yl]carbamoyl]-1H-pyrrolo[2,3-c]pyridine-5-carboxylae (400 mg, 1.13 mmol, 94.11% yield) was obtained as brown solid and was used directly for the next step without purification.

### Synthesis of 2-[[(1S,2S,3S,5R)-2,6,6-trimethylnorpinan-3-yl]carbamoyl]-1H-pyrrolo[2,3-c]pyridine-5-carboxylic acid

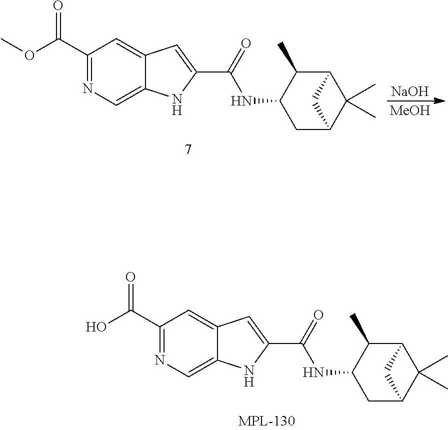

To a solution of methyl 2-[[(1S,2S,3S,5R)-2,6,6-trimethylnorpinan-3-yl]carbamoyl]-1H-pyrrolo[2,3-c]pyridine-5-carboxylate (400 mg, 1.13 mmol, 1 eq) in MeOH (5 mL) was added LiOH (2 M, 4.26 mL, 7.56 eq) (in water), the mixture was stirred at 25° C. for 12 hr. LC-MS showed the starting material 7 was consumed completely and one main peak with desired mass was detected. The mixture was concentrated under reduced pressure to give a residue, extracted with EtOAc (20 Ml×2). The combined inorganic layers were concentrated under reduced pressure to give a residue (2 mL). The residue was purified by prep-HPLC (column: YMC-Actus Triart C18 150*30 5u; mobile phase: [water (0.225% FA)-ACN]; B %: 30%-56%, 11 min). The product 2-[[(1S,2S,3S,5R)-2,6,6-trimethylnorpinan-3-yl]carbamoyl]-1H-pyrrolo[2,3-c]pyridine-5-carboxylic acid (5.2 mg, 14.70 umol, 1.31% yield, 96.482% purity) was obtained as brown solid.

LCMS (ESI) m/z 342.2 [M+H]⁺; ¹H NMR (500 MHz, DMSO-d₆) δ=12.60 (br s, 1H), 8.83 (s, 1H), 8.75 (br d, J=8.3 Hz, 1H), 8.51 (s, 1H), 7.46 (s, 1H), 4.41 (br t, J=8.4 Hz, 1H), 2.45 (br s, 2H), 2.10 (br t, J=7.2 Hz, 1H), 1.96 (br s, 1H), 1.82 (br t, J=5.3 Hz, 1H), 1.73 (br dd, J=7.2, 12.6 Hz, 1H), 1.25-1.20 (m, 4H), 1.09-1.04 (m, 6H).

### Examples 86 and 87. MPL-131 and MPL-133

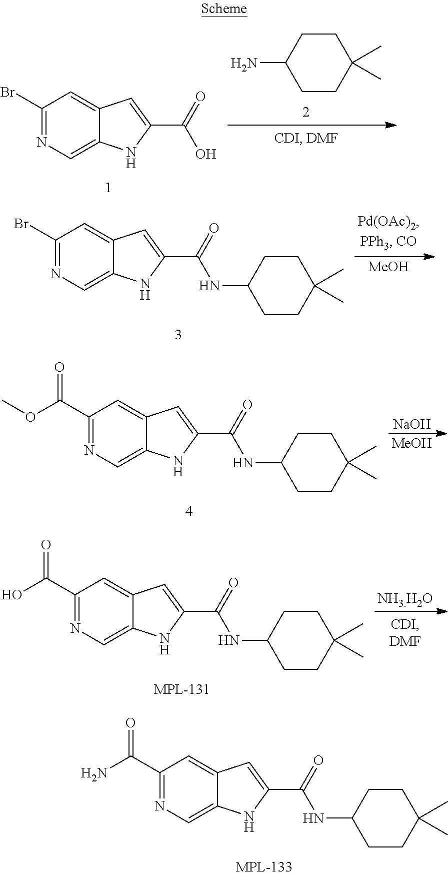

### Synthesis of 5-bromo-N-(4,4-dimethylcyclohexyl)-3aH-pyrrolo[2,3-c]pyridine-2-carboxamide

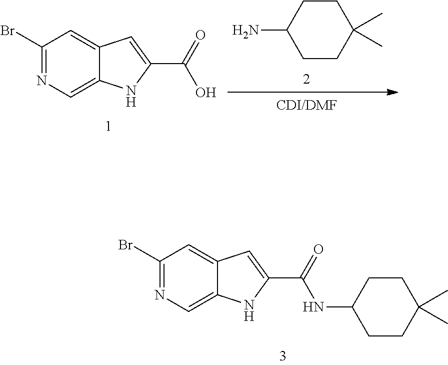

To a solution of 5-bromo-3aH-pyrrolo[2,3-c]pyridine-2-carboxylic acid (500 mg, 2.07 mmol, 1 eq) in DMF (5 mL) was added CDI (504.53 mg, 3.11 mmol, 1.5 eq). The mixture was stirred at 30° C. for 0.5 h. Then 4,4-dimethylcyclohexanamine (395.87 mg, 3.11 mmol, 1.5 eq) was added and the mixture was stirred for another 0.5 h at the same temperature. LCMS showed the desired mass was detected and the reactant 1 was consumed. The mixture was concentrated under reduced pressure to give a residue, then diluted with water (30 mL), acidified with HCl (2 M) to pH=5. The mixture was filtered and the filter cake was washed with 10 mL×3 of Petroleum ether, dried under reduced pressure to give the product. The crude product was used directly for the next step without purification. The product 5-bromo-N-(4,4-dimethylcyclohexyl)-3aH-pyrrolo[2,3-c]pyridine-2-carboxamide (550 mg, 1.56 mmol, 75.37% yield, 99.563% purity) was obtained as brown solid. LCMS (ESI) m/z 350.0 [M]⁺

### Synthesis of 2-[(4,4-dimethylcyclohexyl)carbamoyl]-1H-pyrrolo[2,3-c]pyridine-5-carboxylate

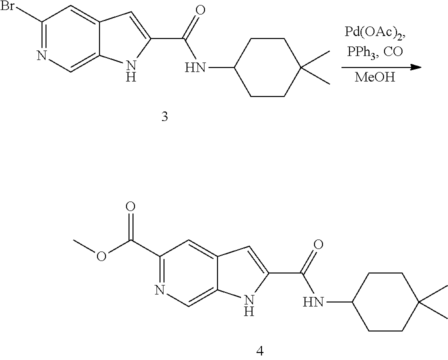

To a solution of 5-bromo-N-(4,4-dimethylcyclohexyl)-1H-pyrrolo[2,3-c]pyridine-2-carboxamide (550 mg, 1.57 mmol, 1 eq) in DMF (3 mL) was added MeOH (3 mL), Pd(OAc)₂ (35.25 mg, 157.03 umol, 0.1 eq), PPh₃ (82.37 mg, 314.06 umol, 0.2 eq) and TEA (794.49 mg, 7.85 mmol, 1.09 mL, 5 eq). The mixture was evacuated 3 times with CO and stirred at 80° C. for 108 hr under carbon monoxide in 3 atm. LC-MS showed one peak with desired mass was detected and the reactant 3 was consumed. The mixture was filtered and the filter was concentrated under reduced pressure to give a residue. The residue was used directly for the next step without purification. The crude product methyl 2-[(4,4-dimethylcyclohexyl)carbamoyl]-1H-pyrrolo[2,3-c]pyridine-5-carboxylate (500 mg, 1.52 mmol, 96.67% yield) was obtained as brown solid and was used directly for the next step without purification. LCMS (ESI) m/z 330.1 [M+H]⁺

### Synthesis of 2-[(4,4-dimethylcyclohexyl)carbamoyl]-1H-pyrrolo[2,3-c]pyridine-5-carboxylic acid

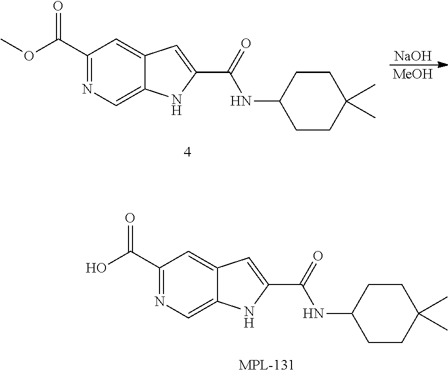

To a solution of methyl 2-[(4,4-dimethylcyclohexyl)carbamoyl]-1H-pyrrolo[2,3-c]pyridine-5-carboxylate (500 mg, 1.52 mmol, 1 eq) (the crude product contained 5-bromo-N-[(1S,2S,3S,5R)-2,6,6-trimethylnorpinan-3-yl]-1H-pyrrolo[2,3-c]pyridine-2-carboxamide 50 mg) in MeOH (5 mL) was added LiOH (2 M, 5.74 mL, 7.56 eq) (in water). The mixture was stirred at 25° C. for 12 hr. LC-MS showed the starting material 4 was consumed completely and one main peak with desired mass was detected. The mixture was concentrated under reduced pressure to give a residue, extracted with EtOAc (20 mL×2), and the combined inorganic layers were concentrated under reduced pressure to give a residue (2 mL). The residue was purified by prep-HPLC (column: YMC-Actus Triart C18 150*30 mm*5 um; mobile phase: [water (0.225% FA)-ACN]; B %: 27%-51%, 11 min) and prep-HPLC (column: Phenomenex Synergi C18 150*30 mm*4 um; mobile phase: [water (0.05% HCl)-ACN]; B %: 19%-49%, 10 min). The product 2-[(4,4-dimethylcyclohexyl)carbamoyl]-1H-pyrrolo[2,3-c]pyridine-5-carboxylic acid (5 mg, 15.84 umol, 1.04% yield, 99.903% purity) was obtained as white solid. Purity comes from LCMS, and the product was confirmed by H NMR. And the product 2 2-[(4,4-dimethylcyclohexyl)carbamoyl]-1H-pyrrolo[2,3-c]pyridine-5-carboxylic acid (350 mg, 1.11 mmol, 73.11% yield) was obtained as white solid.

¹H NMR (400 MHz, DMSO-d₆) δ=13.17 (br s, 1H), 8.90-8.83 (m, 2H), 8.71 (s, 1H), 7.58 (s, 1H), 3.78 (br d, J=7.4 Hz, 1H), 1.73-1.65 (m, 2H), 1.63-1.51 (m, 2H), 1.47-1.38 (m, 2H), 1.35-1.24 (m, 2H), 0.95 (s, 3H).

### Synthesis of N2-(4,4-dimethylcyclohexyl)-1H-pyrrolo[2,3-c]pyridine-2,5-dicarboxamide

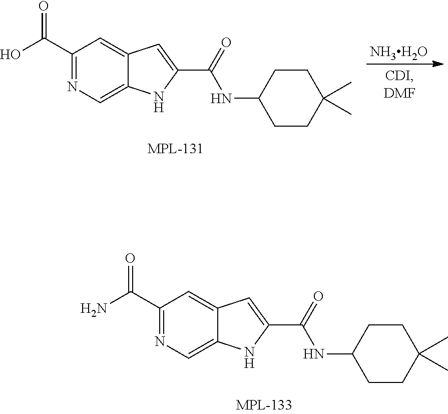

To a solution of 2-[(4,4-dimethylcyclohexyl)carbamoyl]-1H-pyrrolo[2,3-c]pyridine-5-carboxylic acid (80.00 mg, 253.67 umol, 1 eq) in DMF (1.5 mL) was added CDI (61.70 mg, 380.51 umol, 1.5 eq). The mixture was stirred at 25° C. for 0.5 h, then NH₃.H₂O (44.45 mg, 380.51 umol, 48.85 uL, 1.5 eq) was added. The mixture was stirred at 25° C. for 0.5 h. LCMS showed the reaction was consumed and the desired mass was detected. The mixture was added to water (15 mL) and stirred for 5 min, then filtered and the filter cake was dried under reduced pressure to give the crude product. The crude product was purified by prep-HPLC (column: YMC-Actus Triart C18 100*30 mm*5 um; mobile phase: [water (0.225% FA)-ACN]; B %: 31%-54%, 11 min). The product N2-(4,4-dimethylcyclohexyl)-1H-pyrrolo[2,3-c]pyridine-2,5-dicarboxamide (19.2 mg, 60.99 umol, 24.04% yield, 99.872% purity) was obtained as white solid. Purity comes from LCMS. The product was confirmed by ¹H NMR.

LCMS (ESI) m/z 315.2 [M+H]⁺; ¹H NMR (500 MHz, DMSO-d₆) δ=12.33 (s, 1H), 8.75 (s, 1H), 8.55 (d, J=8.1 Hz, 1H), 8.35 (s, 1H), 8.00 (br d, J=2.9 Hz, 1H), 7.44 (br d, J=2.6 Hz, 1H), 7.34 (d, J=1.2 Hz, 1H), 3.81-3.72 (m, 1H), 1.67 (br dd, J=3.8, 13.3 Hz, 2H), 1.61-1.50 (m, 2H), 1.41 (br d, J=12.5 Hz, 2H), 1.33-1.24 (m, 2H), 0.94 (d, J=11.6 Hz, 6H).

### Example 88. MPL-132

### Synthesis of N2-[(1S,2S,3S,5R)-2,6,6-trimethylnorpinan-3-yl]-1H-pyrrolo[2,3-c]pyridine-2,5-dicarboxamide

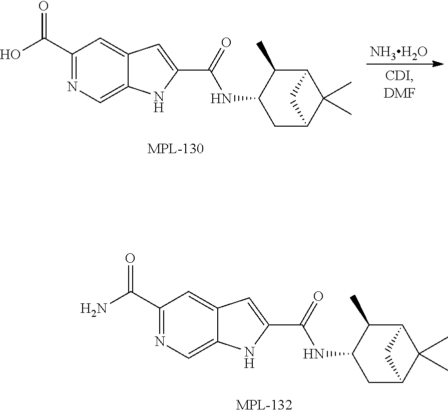

To a solution of 2-[[(1S,2S,3S,5R)-2,6,6-trimethylnorpinan-3-yl]carbamoyl]-1H-pyrrolo[2,3-c]pyridine-5-carboxylic acid (100.00 mg, 292.91 umol, 1 eq) in DMF (2 mL) was added CDI (71.24 mg, 439.36 umol, 1.5 eq). The mixture was stirred at 25° C. for 0.5 h, then NH₃.H₂O (51.33 mg, 439.36 umol, 56.41 uL, 1.5 eq) was added. The mixture was stirred at 25° C. for 0.5 h. LCMS showed the reaction was consumed and the desired mass was detected. The reaction mixture was purified by prep-HPLC (column: YMC-Actus Triart C18 150*30 mm*5 um; mobile phase: [water (0.225% FA)-ACN]; B %: 36%-63%, 11 min) without work up. The product N2-[(1S,2S,3S,5R)-2,6,6-trimethylnorpinan-3-yl]-1H-pyrrolo[2,3-c]pyridine-2,5-dicarboxamide (33.1 mg, 97.02 umol, 33.12% yield, 99.783% purity) was obtained as white solid. Purity comes from LCMS. The product was confirmed by ¹H NMR.

LCMS (ESI) m/z 341.2 [M+H]⁺; ¹H NMR (500 MHz, DMSO-d₆) δ=8.77 (s, 1H), 8.69 (br d, J=8.5 Hz, 1H), 8.40 (s, 1H), 8.06 (br s, 1H), 7.50 (br s, 1H), 7.41 (s, 1H), 4.46-4.35 (m, 1H), 2.48-2.37 (m, 2H), 2.16-2.06 (m, 1H), 1.98-1.93 (m, 1H), 1.82 (t, J=5.2 Hz, 1H), 1.72 (ddd, J=2.1, 6.4, 13.6 Hz, 1H), 1.25-1.20 (m, 4H), 1.09-1.05 (m, 6H).

### Example 89. MPL-134

### Synthesis of 5-fluoro-7-methyl-N-[(1S,2S,3S,5R)-2,6,6-trimethylnorpinan-3-yl]-1H-pyrrolo[2,3-c]pyridine-2-carboxamide

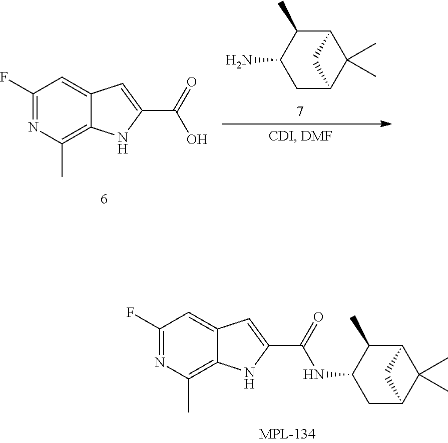 [SMILES: Cc1nc(F)cc2cc(C(=O)O)[nH]c12 ; C[C@@H]1[C@@H](N)C[C@H]2C[C@@H]1C2(C)C ; Cc1nc(F)cc2cc(C(=O)N[C@H]3C[C@H]4C[C@@H]([C@@H]3C)C4(C)C)[nH]c12]

To a solution of 5-fluoro-7-methyl-1H-pyrrolo[2,3-c]pyridine-2-carboxylic acid (40 mg, 206.01 umol, 1 eq) in DMF (1.5 mL) was added CDI (50.11 mg, 309.02 umol, 1.5 eq), the mixture was stirred at 30° C. for 0.5 h, then (1S,2S,3S,5R)-2,6,6-trimethylnorpinan-3-amine (47.36 mg, 309.02 umol, 1.5 eq) was added and the mixture was stirred for another 0.5 h at the same temperature. LCMS showed the desired mass was detected. The mixture was dropwise added to water (15 mL), and stirred for 10 min, filtered and the filter cake was dried under reduced pressure to give the crude product. The residue was purified by prep-HPLC (column: YMC-Actus Triart C18 150*30 mm*5 um; mobile phase: [water (0.225% FA)-ACN]; B %: 51%-81%, 11 min). The product 5-fluoro-7-methyl-N-[(1S,2S,3S,5R)-2,6,6-trimethylnorpinan-3-yl]-1H-pyrrolo[2,3-c]pyridine-2-carboxamide (23.6 mg, 71.64 umol, 34.78% yield, 100% purity) was obtained as white solid.

LCMS (ESI) m/z 330.2 [M+H]⁺; ¹H NMR (400 MHz, DMSO-δ₆)=12.05 (s, 1H), 8.57 (d, J=8.6 Hz, 1H), 7.18 (d, J=1.7 Hz, 1H), 7.09 (s, 1H), 4.39 (br s, 1H), 2.68 (s, 3H), 2.47-2.32 (m, 2H), 2.08 (s, 1H), 1.94 (br d, J=2.4 Hz, 1H), 1.86-1.79 (m, 1H), 1.72 (ddd, J=2.0, 6.5, 13.6 Hz, 1H), 1.25-1.19 (m, 4H), 1.07 (t, J=3.5 Hz, 6H).

### Example 90. MPL-135

### Synthesis of N-(4,4-dimethylcyclohexyl)-5-fluoro-7-methyl-1H-pyrrolo[2,3-c]pyridine-2-carboxamide

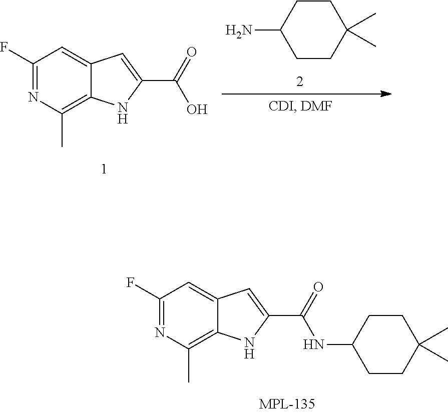

To a solution of 5-fluoro-7-methyl-1H-pyrrolo[2,3-c]pyridine-2-carboxylic acid (50 mg, 257.52 umol, 1 eq) in DMF (1.5 mL) was added CDI (62.63 mg, 386.27 umol, 1.5 eq). The mixture was stirred at 30° C. for 0.5 h, then 4,4-dimethylcyclohexanamine (49.14 mg, 386.27 umol, 1.5 eq) was added. The mixture was stirred for another 0.5 h at the same temperature. LCMS showed the desired mass was detected. The mixture was dropwise added to water (15 mL), and stirred for 10 min, filtered and the filter cake was dried under reduced pressure to give the crude product. The product N-(4,4-dimethylcyclohexyl)-5-fluoro-7-methyl-1H-pyrrolo[2,3-c]pyridine-2-carboxamide (56.6 mg, 186.57 umol, 72.45% yield, 100% purity) was obtained as white solid.

LCMS (ESI) m/z 304.2 [M+H]⁺; ¹H NMR (400 MHz, DMSO-δ₆)=12.02 (br s, 1H), 8.43 (br d, J=8.3 Hz, 1H), 7.14 (s, 1H), 7.07 (s, 1H), 3.82-3.67 (m, 1H), 2.67 (s, 3H), 1.72-1.63 (m, 2H), 1.60-1.49 (m, 2H), 1.45-1.37 (m, 2H), 1.33-1.23 (m, 2H), 0.94 (d, J=8.1 Hz, 6H).

### Example 91. MPL-138

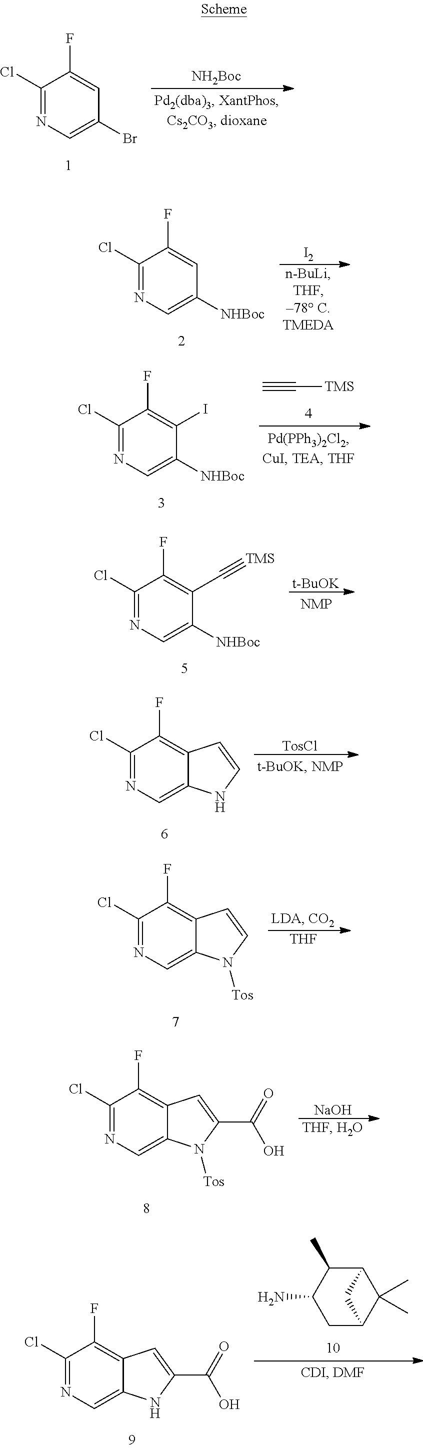

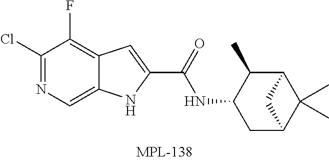 [SMILES: C[C@@H]1[C@@H](NC(=O)c2cc3c(F)c(Cl)ncc3[nH]2)C[C@H]2C[C@@H]1C2(C)C]

### 5-chloro-4-fluoro-1-(p-tolylsulfonyl)pyrrolo[2,3-c]pyridine

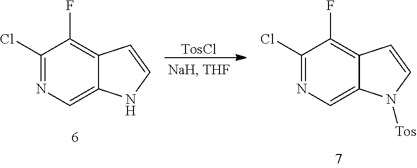

To a solution of 5-chloro-4-fluoro-1H-pyrrolo[2,3-c]pyridine (770 mg, 4.51 mmol, 1 eq) in THF (10 mL) was added NaH (270.83 mg, 6.77 mmol, 60% purity, 1.5 eq). The mixture was stirred at 0° C. for 10 minutes. Then TosCl (1.72 g, 9.03 mmol, 2 eq) was added. The mixture was stirred at 25° C. for 12 hr under N₂ atmosphere. TLC and LCMS showed the starting material was consumed completely. The reaction mixture was quenched by addition saturated aqueous NH₄Cl (20 mL). The mixture was concentrated in reduced pressure and diluted with EtOAc (150 mL), The organic phase was washed with brine (50 mL×3), dried with anhydrous Na₂SO₄, filtered and concentrated in vacuo. The crude product was purified by silica column chromatography (0-10% EtOAc/Petroleum ether gradient, 20 g silica column). All fractions were combined and evaporated. Compound 5-chloro-4-fluoro-1-(p-tolylsulfonyl)pyrrolo[2,3-c]pyridine (1.1 g, 3.32 mmol, 73.53% yield, 98% purity) was obtained as a yellow solid. LCMS (ESI) m/z 324.9 [M+H]⁺

### 5-chloro-4-fluoro-1-(p-tolylsulfonyl)pyrrolo[2,3-c]pyridine-2-carboxylic acid

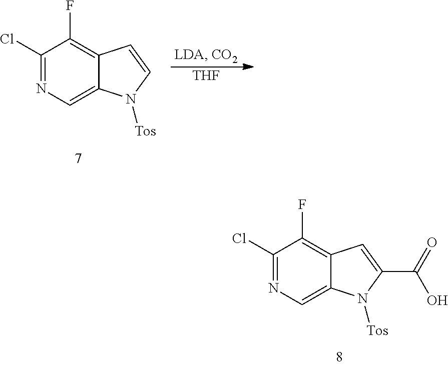

To a solution of 5-chloro-4-fluoro-1-(p-tolylsulfonyl)pyrrolo[2,3-c]pyridine (1.1 g, 3.39 mmol, 1 eq) in THF (15 mL) was added LDA (2 M, 2.54 mL, 1.5 eq) at −78° C. in 1 hr under N₂ atmosphere. Then, the mixture was stirred in −78° C. under CO₂ (149.07 mg, 3.39 mmol, 1 eq) for 0.5 hr. LCMS and TLC showed there were no starting materials and one main peak with desired mass was detected. The reaction was quenched with saturated aqueous NH₄Cl (20 mL) concentrated under reduced pressure to removed THF, then acidified with HCl (2 M) to pH=5, extracted with EtOAc (20 mL×3). The mixture was filtered through a Celite pad, and the filtrate cake was concentrated to give the crude product. The crude product was purified by silica column chromatography (eluent of 10-25% EtOAc/Petroleum ether gradient, 20 g silica column). All fractions found to contain product by TLC (Petroleum ether:EtOAc=1:1, Rf=0.2) were combined and evaporated. Compound 5-chloro-4-fluoro-1-(p-tolylsulfonyl)pyrrolo[2,3-c]pyridine-2-carboxylic acid (0.98 g, 2.55 mmol, 75.32% yield, 96% purity) was obtained as a white solid. LCMS (ESI) m/z 368.9 [M+H]⁺

### 5-chloro-4-fluoro-1H-pyrrolo[2,3-c]pyridine-2-carboxylic acid

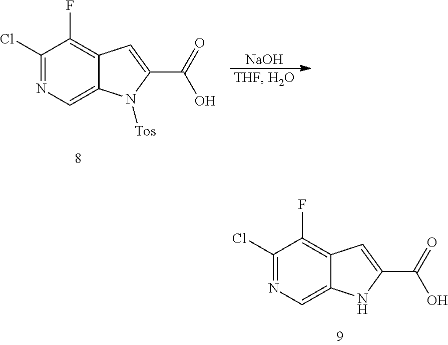

To a solution of 5-chloro-4-fluoro-1-(p-tolylsulfonyl)pyrrolo[2,3-c]pyridine-2-carboxylic acid (560 mg, 1.52 mmol, 1 eq) in NaOH (2 M, 3.50 mL, 4.61 eq). and THF (3 mL). The mixture was stirred at 75° C. for 3 hrs. LCMS showed there were no starting materials and main desired compound. The mixture was concentrated under reduced pressure to give a residue, then acidified with HCl (2 M) to pH=5. The mixture was filtered through a Celite pad, and the filtrate cake was concentrated to give the crude product. The residue was used directly for next step without further purification. Compound 5-chloro-4-fluoro-1H-pyrrolo[2,3-c]pyridine-2-carboxylic acid (240 mg, 1.12 mmol, 73.65% yield, 100% purity) was obtained as a white solid. LCMS (ESI) m/z 215.0 [M+H]⁺

### 5-chloro-4-fluoro-N-[(1S,2S,3S,5R)-2,6,6-trimethylnorpinan-3-yl]-1H-pyrrolo[2,3-c]pyridine-2-carboxamide

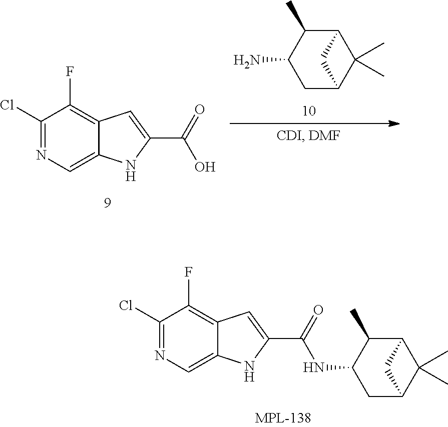 [SMILES: O=C(O)c1cc2c(F)c(Cl)ncc2[nH]1 ; C[C@@H]1[C@@H](N)C[C@H]2C[C@@H]1C2(C)C ; C[C@@H]1[C@@H](NC(=O)c2cc3c(F)c(Cl)ncc3[nH]2)C[C@H]2C[C@@H]1C2(C)C]

To a solution of (1S,2S,3S,5R)-2,6,6-trimethylnorpinan-3-amine (42.85 mg, 279.61 umol, 1.2 eq) and CDI (75.56 mg, 466.02 umol, 2 eq) was added in DMF (1 mL). The mixture was stirred at 30° C. for 0.5 hr, then 5-chloro-4-fluoro-1H-pyrrolo[2,3-c]pyridine-2-carboxylic acid (50 mg, 233.01 umol, 1 eq) was added under N₂ atmosphere. The mixture was stirred at 30° C. for 3 hrs. LCMS showed there was no starting material and main desired compound. The mixture was added in water (10 mL) and stirred for 10 mins. The mixture was extracted with EtOAc (15 mL×3). The organic phase was washed with saturated brine (4 mL×3). The mixture was dried with anhydrous Na₂SO₄, filtered and concentrated in vacuo. The crude product was purified by reversed-phase HPLC (column: YMC-Actus Triart C18 150*30 mm*5 um; mobile phase: [water (0.225% FA)-ACN]; B %: 55%-84%, 11 min). Compound 5-chloro-4-fluoro-N-[(1S,2S,3S,5R)-2,6,6-trimethylnorpinan-3-yl]-1H-pyrrolo[2,3-c]pyridine-2-carboxamide (9.2 mg, 25.51 umol, 10.95% yield, 97% purity) was obtained as a white solid.

LCMS (ESI) m/z 350.1 [M+H]⁺; ¹H NMR (400 MHz, DMSO-d₆) δ=1.02-1.10 (m, 1H) 1.06 (s, 3H) 1.19 (br d, J=9.54 Hz, 1H) 1.23 (s, 3H) 1.71 (br dd, J=11.86, 6.24 Hz, 1H) 1.81 (br t, J=5.01 Hz, 1H) 1.90-2.00 (m, 1H) 2.08 (br t, J=7.34 Hz, 1H) 2.29-2.45 (m, 1H) 2.29-2.45 (m, 1H) 4.29-4.47 (m, 1H) 7.42 (s, 1H) 8.46 (s, 1H) 8.70 (br d, J=8.56 Hz, 1H) 12.64 (br s, 1H).

### Example 92. MPL-139

### 5-chloro-N-(4,4-dimethylcyclohexyl)-4-fluoro-1H-pyrrolo[2,3-c]pyridine-2-carboxamide

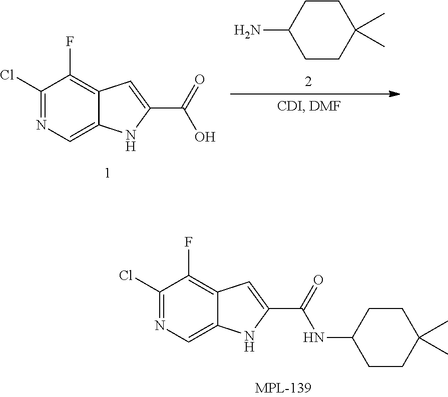

To a solution of 5-chloro-4-fluoro-1H-pyrrolo[2,3-c]pyridine-2-carboxylic acid (40 mg, 186.41 umol, 1 eq) and CDI (60.45 mg, 372.82 umol, 2 eq) was added in DMF (1 mL). The mixture was stirred at 25° C. for 0.5 h, then 4,4-dimethylcyclohexanamine (28.46 mg, 223.69 umol, 1.2 eq) was added under N₂ atmosphere. The mixture was stirred at 25° C. for 3 h under N₂ atmosphere. LCMS showed starting material consumed and no desired product. The mixture was added in water (10 mL) and stirred for 10 mins. The mixture was extracted with EtOAc (15 mL×3). The organic phase was washed with brine (4 mL×3). The mixture was dried with anhydrous Na₂SO₄, filtered and concentrated in vacuo. The crude product was purified by reverse-phase HPLC (column: YMC-Actus Triart C18 150*30 mm*5 um; mobile phase: [water (0.225% FA)-ACN]; B %: 52%-77%, 11 min). Compound 5-chloro-N-(4,4-dimethylcyclohexyl)-4-fluoro-1H-pyrrolo[2,3-c]pyridine-2-carboxamide (8.5 mg, 25.99 umol, 13.94% yield, 99% purity) was obtained as a white solid.

LCMS (ESI) m/z 324.1 [M+H]⁺; ¹H NMR (400 MHz, DMSO-d6) δ=1.00-1.12 (m, 6H) 1.19 (br d, J=9.54 Hz, 1H) 1.23 (s, 3H) 1.70 (br dd, J=12.10, 6.48 Hz, 1H) 1.81 (br t, J=5.26 Hz, 1H) 1.94 (br s, 1H) 2.07 (br t, J=7.21 Hz, 1H) 2.30-2.45 (m, 2H) 2.45-2.55 (m, 31H) 4.36 (quin, J=8.01 Hz, 1H) 4.28-4.45 (m, 1H) 7.22 (s, 1H) 8.48 (s, 1H) 8.53 (br d, J=8.56 Hz, 1H) 12.54 (br s, 1H).

### Example 93. MPL-141

### Synthesis of N-(4,4-dimethylcyclohexyl)-4-fluoro-5-methyl-1H-pyrrolo[2,3-c]pyridine-2-carboxamide

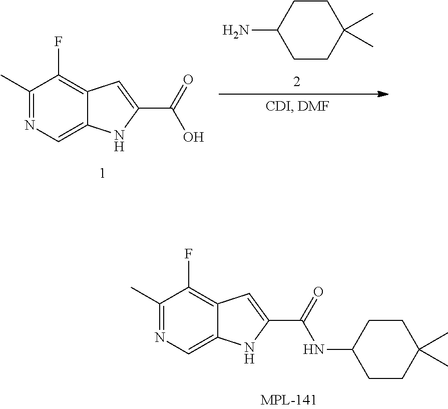

To a solution of 4-fluoro-5-methyl-1H-pyrrolo[2,3-c]pyridine-2-carboxylic acid (0.1 g, 515.03 umol, 1 eq) in DMF (5 mL, dried by CaH₂) was added CDI (100.21 mg, 618.04 umol, 1.2 eq). The mixture was stirred at 20° C. for 0.5 hr. Then 4,4-dimethylcyclohexanamine (78.63 mg, 618.04 umol, 1.2 eq) was added, the mixture was stirred at 20° C. for 1 hr. LC-MS showed Reactant was consumed completely and one main peak with desired mass was detected. The reaction mixture was dropped into water (20 mL). The product was isolated as white solid. Filtered, the filter cake was redissolved in DMF (8 mL), and then purified by prep-HPLC (FA condition, column: YMC-Actus Triart C18 150*30 mm*5 um; mobile phase: [water (0.225% FA)-ACN]; B %: 30%-55%, 11 min). Compound N-(4,4-dimethylcyclohexyl)-4-fluoro-5-methyl-1H-pyrrolo[2,3-c]pyridine-2-carboxamide (18 mg, 59.33 umol, 11.52% yield, 100% purity) was obtained as a white solid.

LCMS (ESI) m/z 303.17 [M+H]⁺; ¹H NMR (500 MHz, DMSO-d₆) δ=0.95 (d, J=10.68 Hz, 6H) 1.26-1.35 (m, 2H) 1.43 (br d, J=12.36 Hz, 2H) 1.51-1.61 (m, 2H) 1.69 (br dd, J=13.12, 3.81 Hz, 2H) 2.49 (d, J=3.05 Hz, 3H) 3.72-3.81 (m, 1H) 7.29 (s, 1H) 8.53 (br d, J=7.93 Hz, 1H) 8.56 (s, 1H) 12.38 (br s, 1H).

### Example 94. MPL-154

### Synthesis of 4-fluoro-N-spiro[2.5]octan-6-yl-1H-pyrrolo[2,3-b]pyridine-2-carboxamide

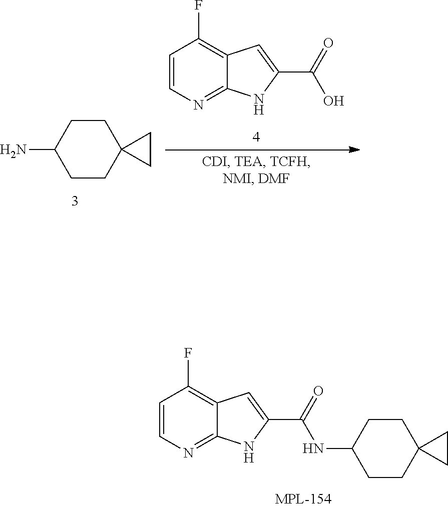 [SMILES: O=C(O)c1cc2c(F)ccnc2[nH]1 ; NC1CCC2(CC1)CC2 ; O=C(NC1CCC2(CC1)CC2)c1cc2c(F)ccnc2[nH]1]

To a solution of 4-fluoro-1H-pyrrolo[2,3-b]pyridine-2-carboxylic acid (50 mg, 277.57 umol, 1 eq) in DMF (1 mL) was added TEA (84.26 mg, 832.71 umol, 115.90 uL, 3 eq) and CDI (58.51 mg, 360.84 umol, 1.3 eq). The mixture was stirred at 30° C. for 0.5 hr. Spiro[2.5]octan-6-amine (53.85 mg, 333.08 umol, 1.2 eq, HCl) was added and the mixture was stirred at 30° C. for another 12 h. LCMS showed there were starting material and main desired compound. [chloro(dimethylamino)methylene]-dimethyl-ammonium; hexafluorophosphate (101.24 mg, 360.84 umol, 1.3 eq) and 1-methylimidazole (68.37 mg, 832.71 umol, 66.38 uL, 3 eq) was added and the mixture was stirred at 30° C. for 12 hr. LCMS showed there were no starting material and main desired compound. The reaction was added dropwise to H₂O (20 mL). There was much precipitation which was collected by filter. The cake was diluted in EtOAc (20 mL), dried with anhydrous MgSO₄, filtered. The filtrate was concentrated in vacuo. The residue was purified by column chromatography (SiO₂, Petroleum ether:EtOAc=10:1 to 1:1). Then the residue was purified by prep-HPLC (column: YMC-Actus Triart C18 150*30 mm*5 um; mobile phase: [water (0.225% FA)-ACN]; B %: 44%-70%, 11 min). Compound 4-fluoro-N-spiro[2.5]octan-6-yl-1H-pyrrolo[2,3-b]pyridine-2-carboxamide (20 mg, 69.61 umol, 25.08% yield, 100% purity) was obtained as a white solid which was confirmed by LCMS and ¹H NMR.

LCMS (ESI) m/z 288.1 [M+H]⁺; ¹H NMR (500 MHz, METHANOL-d₄)=8.32 (br t, J=6.4 Hz, 1H), 8.35-8.28 (m, 1H), 7.22 (s, 1H), 6.94 (br dd, J=5.6, 9.7 Hz, 1H), 3.95 (br t, J=11.2 Hz, 1H), 2.00-1.84 (m, 4H), 1.65-1.55 (m, 2H), 1.01 (br d, J=12.8 Hz, 2H), 0.34 (br d, J=7.2 Hz, 2H), 0.30 (br d, J=6.7 Hz, 2H).

### Example 95. MPL-155

### N-(2,2-difluorospiro[2.5]octan-6-yl)-4-fluoro-1H-pyrrolo[2,3-b]pyridine-2-carboxamide

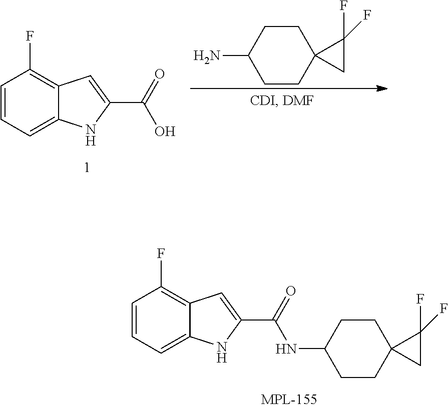 [SMILES: O=C(O)c1cc2c(F)cccc2[nH]1 ; NC1CCC2(CC1)CC2(F)F ; O=C(NC1CCC2(CC1)CC2(F)F)c1cc2c(F)cccc2[nH]1]

To a solution of 4-fluoro-1H-pyrrolo[2,3-b]pyridine-2-carboxylic acid (50.28 mg, 279.10 umol, 1 eq) in DMF (1 mL) was added CDI (90.51 mg, 558.20 umol, 2 eq) and TEA (56.48 mg, 558.20 umol, 77.69 uL, 2 eq) under N₂ atmosphere. The mixture was stirred at 30° C. for 0.5 hr under N₂ atmosphere. Then 2,2-difluorospiro[2.5]octan-6-amine (53.99 mg, 273.24 umol, 9.79e-1 eq, HCl) was added. The mixture was stirred at 30° C. for 2 hr under N₂ atmosphere. LCMS showed the starting material was still existed and main desired compound. The mixture was added in water (10 mL) and stirred for 10 mins. The mixture was extracted with EtOAc (15 mL×3). The organic phase was washed with saturated brine (15 mL×3). The mixture was dried with anhydrous Na₂SO₄, filtered and concentrated in vacuo. The crude product was purified by reversed-phase HPLC (column: YMC-Actus Triart C18 150*30 mm*5 um; mobile phase: [water (0.225% FA)-ACN]; B %: 45%-70%, 11 min). Compound N-(2,2-difluorospiro[2.5]octan-6-yl)-4-fluoro-1H-pyrrolo[2,3-b]pyridine-2-carboxamide (7.2 mg, 21.16 umol, 7.58% yield, 95% purity) was obtained as a white solid.

LCMS (ESI) m/z 324.1 [M+H]⁺; ¹H NMR (500 MHz, DMSO-d6) δ=1.25 (br t, J=8.54 Hz, 2H) 1.37-1.62 (m, 4H) 1.68-1.82 (m, 2H) 1.89 (br dd, J=12.44, 3.28 Hz, 2H) 3.83-3.98 (m, 1H) 7.01 (dd, J=10.22, 5.34 Hz, 1H) 7.27 (d, J=1.83 Hz, 1H) 8.32 (dd, J=8.32, 5.42 Hz, 1H) 8.40 (br d, J=7.78 Hz, 1H) 12.48 (br s, 1H).

### Example 96. MPL-157

### Synthesis of N-(4,4-dimethylcyclohexyl)-5-methyl-1H-pyrrolo[2,3-c]pyridine-2-carboxamide

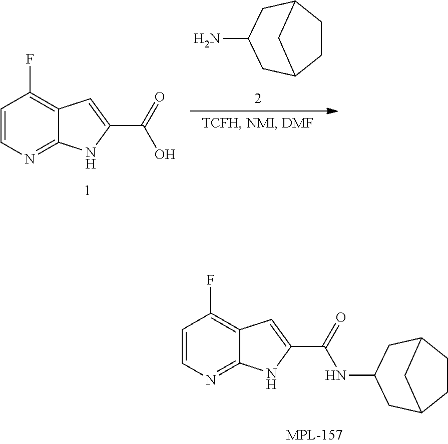 [SMILES: O=C(O)c1cc2c(F)ccnc2[nH]1 ; NC1CC2CCC(C1)C2 ; O=C(NC1CC2CCC(C2)C1)c1cc2c(F)ccnc2[nH]1]

To a solution of 4-fluoro-1H-pyrrolo[2,3-b]pyridine-2-carboxylic acid (30.39 mg, 168.69 umol, 1 eq) in DMF (0.5 mL) was added bicyclo[3.2.1]octan-3-amine (30 mg, 185.56 umol, 1.1 eq, HCl), 1-methylimidazole (55.40 mg, 674.77 umol, 53.79 uL, 4 eq) and [chloro(dimethylamino)methylene]-dimethyl-ammonium; hexafluorophosphate (61.53 mg, 219.30 umol, 1.3 eq). The mixture was stirred at 30° C. for 2 hr. LCMS showed there were no starting material and main desired compound. The reaction was added dropwise to H₂O (20 mL). There was much precipitation which was collected by filter. The cake was diluted in EtOAc (20 mL), dried with anhydrous MgSO₄, filtered. The filtrate was concentrated in vacuo. The residue was purified by prep. HPLC (column: YMC-Actus Triart C18 150*30 mm*5 um; mobile phase: [water (0.225% FA)-ACN]; B %: 45%-74%, 11 min). Compound N-(3-bicyclo[3.2.1]octanyl)-4-fluoro-1H-pyrrolo[2,3-b]pyridine-2-carboxamide (10 mg, 34.36 umol, 20.37% yield, 98.72% purity) was obtained as a white solid.

LCMS (ESI) m/z 288.1 [M+H]⁺; ¹H NMR (400 MHz, METHANOL-d4) 8.31 (dd, J=5.7, 7.9 Hz, 1H), 7.18 (s, 1H), 6.92 (dd, J=5.5, 9.9 Hz, 1H), 4.34-4.22 (m, 1H), 2.30 (br s, 2H), 1.91-1.83 (m, 2H), 1.78-1.65 (m, 4H), 1.53-1.42 (m, 4H).

### Example 97. MPL-158

### Synthesis of 4-fluoro-N-(4-fluoro-4-methyl-cyclohexyl)-1H-pyrrolo[2,3-b]pyridine-2-carboxamide

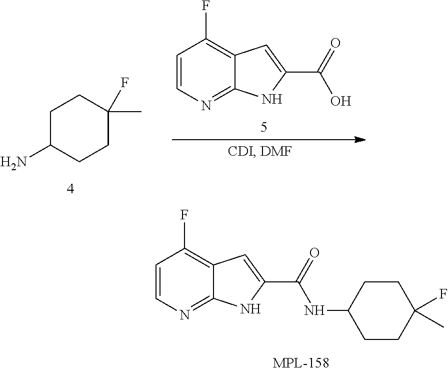

To a solution of 4-fluoro-1H-pyrrolo[2,3-b]pyridine-2-carboxylic acid (100 mg, 555.14 umol, 1 eq) in DMF (1 mL) was added 4-fluoro-4-methyl-cyclohexanamine (120.99 mg, 721.68 umol, 1.3 eq, HCl), 1-methylimidazole (227.88 mg, 2.78 mmol, 221.25 uL, 5 eq) and [chloro(dimethylamino)methylene]-dimethyl-ammonium; hexafluorophosphate (233.64 mg, 832.71 umol, 1.5 eq). The mixture was stirred at 30° C. for 2 hr. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. The crude product was purified by prep-HPLC (column: YMC-Actus Triart C18 150*30 5u; mobile phase: [water (0.225% FA)-ACN]; B %: 45%-70%, 11 min). Compound 4-fluoro-N-(4-fluoro-4-methyl-cyclohexyl)-1H-pyrrolo[2,3-b]pyridine-2-carboxamide (40 mg, 133.80 umol, 24.10% yield, 98.11% purity) was obtained as a white solid which was confirmed by LCMS and ¹H NMR.

LCMS (ESI) m/z 294.2 [M+H]⁺; ¹H NMR (400 MHz, DMSO-d₆) δ=12.54-12.40 (m, 1H), 8.38-8.27 (m, 1H), 8.20 (br d, J=7.3 Hz, 1H), 7.23 (d, J=2.0 Hz, 1H), 7.03-6.95 (m, 1H), 3.97-3.94 (m, 0.8H), 3.81 (br s, 0.2H), 1.91-1.73 (m, 4H), 1.73-1.62 (m, 2H), 1.54 (q, J=8.5 Hz, 2H), 1.39 (s, 1.2H), 1.36-1.30 (m, 1.5H), 1.27 (s, 0.3H).

### Example 98. MPL-161

### Synthesis of N-(4-bicyclo[2.2.2]octanyl)-4-fluoro-1H-pyrrolo[2,3-b]pyridine-2-carboxamide

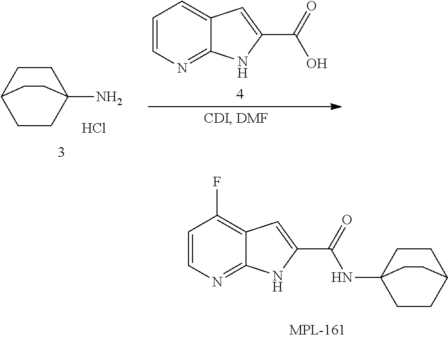 [SMILES: NC12CCC(CC1)CC2 ; O=C(O)c1cc2cccnc2[nH]1 ; Cl ; O=C(NC12CCC(CC1)CC2)c1cc2c(F)ccnc2[nH]1]

To a solution of 4-fluoro-1H-pyrrolo[2,3-b]pyridine-2-carboxylic acid (50 mg, 277.57 umol, 1 eq) and bicyclo[2.2.2]octan-4-amine (67.31 mg, 416.35 umol, 1.5 eq, HCl) in DMF (1 mL) was added HOBt (56.26 mg, 416.35 umol, 1.5 eq) and EDCI (79.81 mg, 416.35 umol, 1.5 eq) TEA (84.26 mg, 832.71 umol, 115.90 uL, 3 eq), the mixture was stirred at 25° C. for 4 hr under N₂. LC-MS showed the starting material 3 was consumed completely and one main peak with desired mass was detected. The mixture was added to water (20 mL) and stirred for 10 min, filtered and the filter cake was dried under reduced pressure. The product N-(4-bicyclo[2.2.2]octanyl)-4-fluoro-1H-pyrrolo[2,3-b]pyridine-2-carboxamide (68.2 mg, 232.82 umol, 83.88% yield, 98.090% purity) was obtained as a white solid.

LCMS (ESI) m/z 288.1 [M+H]⁺; ¹H NMR (500 MHz, METHANOL-d4)=12.37 (br s, 1H), 8.31 (dd, J=5.4, 8.6 Hz, 1H), 7.66 (s, 1H), 7.23 (s, 1H), 6.99 (dd, J=5.4, 10.3 Hz, 1H), 1.98-1.88 (m, 6H), 1.68-1.59 (m, 6H), 1.58-1.53 (m, 1H).

### Example 99. MPL-163

### Synthesis of 4-chloro-N-spiro[2.5]octan-6-yl-1H-pyrrolo[2,3-c]pyridine-2-carboxamide

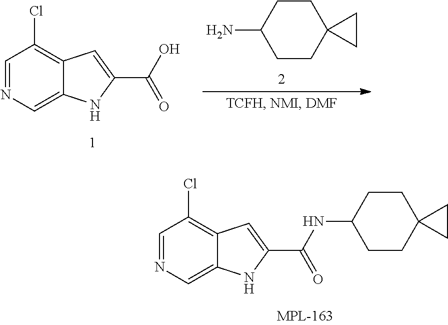 [SMILES: O=C(O)c1cc2c(Cl)cncc2[nH]1 ; NC1CCC2(CC1)CC2 ; O=C(NC1CCC2(CC1)CC2)c1cc2c(Cl)cncc2[nH]1]

To a solution of spiro[2.5]octan-6-amine (53.85 mg, 333.08 umol, 1.2 eq, HCl) in DMF (1 mL) was added 4-chloro-1H-pyrrolo[2,3-c]pyridine-2-carboxylic acid (54.57 mg, 277.57 umol, 1 eq), 1-methylimidazole (91.15 mg, 1.11 mmol, 88.50 uL, 4 eq) and [chloro(dimethylamino)methylene]-dimethyl-ammonium; hexafluorophosphate (116.82 mg, 416.35 umol, 1.5 eq). The mixture was stirred at 30° C. for 12 hr. LCMS showed there were starting material and main desired compound. The reaction was added dropwise to H₂O (20 mL). There was much precipitation which was collected by filter. The cake was diluted in EtOAc (20 mL) and concentrated in vacuo. The residue was purified by prep-HPLC (column: YMC-Actus Triart C18 150*30 mm*5 um; mobile phase: [water (0.225% FA)-ACN]; B %: 31%-60%, 11 min). Compound 4-chloro-N-spiro[2.5]octan-6-yl-1H-pyrrolo[2,3-c]pyridine-2-carboxamide (30 mg, 98.32 umol, 35.42% yield, 99.56% purity) was obtained as a white solid.

LCMS (ESI) m/z 304.1 [M+H]⁺; ¹H NMR (400 MHz, METHANOL-d4) 8.74 (s, 1H), 8.15 (s, 1H), 7.33 (s, 1H), 4.01-3.91 (m, 1H), 1.98-1.93 (m, 2H), 1.93-1.84 (m, 2H), 1.67-1.58 (m, 2H), 1.01 (br d, J=13.6 Hz, 2H), 0.37-0.33 (m, 2H), 0.31-0.26 (m, 2H).

### Example 100. MPL-164

### Synthesis of 4-chloro-N-(1,1-difluorospiro[2.5]octan-6-yl)-1H-pyrrolo[2,3-c]pyridine-2-carboxamide

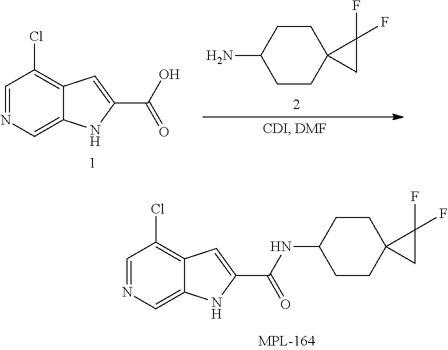 [SMILES: O=C(O)c1cc2c(Cl)cncc2[nH]1 ; NC1CCC2(CC1)CC2(F)F ; O=C(NC1CCC2(CC1)CC2(F)F)c1cc2c(Cl)cncc2[nH]1]

To a solution of 4-chloro-1H-pyrrolo[2,3-c]pyridine-2-carboxylic acid (50 mg, 254.34 umol, 1 eq) in DMF (1 mL) was added CDI (49.49 mg, 305.20 umol, 1.2 eq). The mixture was stirred at 30° C. for 0.5 hr. 2,2-difluorospiro[2.5]octan-6-amine (60.32 mg, 305.20 umol, 1.2 eq, HCl) was added. The mixture was stirred at 30° C. for 1.5 hr under N₂. LC-MS showed reactant 1 was consumed completely and one main peak with desired mass was detected. The reaction mixture was added to H₂O (10 mL) and stirred for 10 min, then extracted with EtOAc (30 mL×3). The combined organic layers was dried over Na₂SO₄, filtered and concentrated under reduced pressure to give a residue. Compound 4-chloro-N-(2,2-difluorospiro[2.5]octan-6-yl)-1H-pyrrolo[2,3-c]pyridine-2-carboxamide (5.3 mg, 15.57 umol, 6.12% yield, 99.833% purity) was obtained as a white solid.

LCMS (ESI) m/z 340.1 [M+H]⁺; ¹H NMR (400 MHz, METHANOL-d₄)=8.72 (s, 1H), 8.13 (s, 1H), 7.31 (s, 1H), 4.05-3.94 (m, 1H), 2.03 (br d, J=9.0 Hz, 2H), 1.83 (br t, J=10.9 Hz, 2H), 1.69-1.45 (m, 4H), 1.17-1.08 (m, 2H).

### Example 101. MPL-167

### 4-chloro-N-(4-fluoro-4-methyl-cyclohexyl)-1H-pyrrolo[2,3-c]pyridine-2-carboxamide

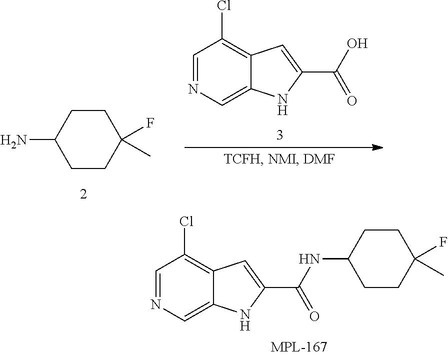

To a solution of 4-chloro-1H-pyrrolo[2,3-c]pyridine-2-carboxylic acid (100 mg, 508.67 umol, 1 eq) in DMF (1 mL) was added 1-methylimidazole (208.82 mg, 2.54 mmol, 202.74 uL, 5 eq). [chloro(dimethylamino)methylene]-dimethyl-ammonium; hexafluorophosphate (214.08 mg, 763.01 umol, 1.5 eq) and 4-fluoro-4-methyl-cyclohexanamine (110.86 mg, 661.27 umol, 1.3 eq, HCl). The mixture was stirred at 25° C. for 12 h. LCMS showed there were no starting material and main desired compound. The reaction mixture was added to water (15 mL), then filtered and the filter cake was washed with 10 mL of water, dried in vacuo to give crude product. The crude product was purified by prep-HPLC (column: YMC-Actus Triart C18 150*30 5u; mobile phase: [water (0.225% FA)-ACN]; B %: 28%-55%, 11 min). The product 4-chloro-N-(4-fluoro-4-methyl-cyclohexyl)-1H-pyrrolo[2,3-c]pyridine-2-carboxamide (20 mg, 64.11 umol, 12.60% yield, 99.3% purity) was obtained as white solid.

LCMS (ESI) m/z 310.1 [M+H]⁺; ¹H NMR (500 MHz, DMSO-d₆) δ=12.61 (br s, 1H), 8.76 (s, 1H), 8.70 (d, J=8.2 Hz, 0.3H), 8.54 (d, J=7.6 Hz, 0.7H), 8.23 (s, 1H), 7.41-7.38 (m, 1H), 4.03-3.97 (m, 0.7H), 3.88 (br s, 0.3H), 1.89-1.83 (m, 3H), 1.77-1.63 (m, 3H), 1.63-1.54 (m, 2H), 1.42 (s, 1H), 1.38 (s, 1H), 1.35 (s, 0.5H), 1.30 (s, 0.5H).

### Example 102. MPL-169

### 4-chloro-N-(4,4-dimethylcyclohex-2-en-1-yl)-1H-pyrrolo[2,3-c]pyridine-2-carboxamide

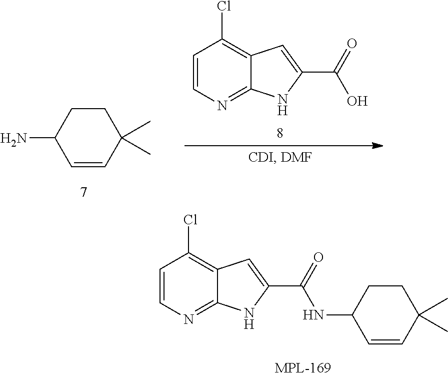

To a solution of 4-chloro-1H-pyrrolo[2,3-c]pyridine-2-carboxylic acid (50 mg, 254.34 umol, 1 eq) in DMF (1 mL) was added CDI (82.48 mg, 508.67 umol, 2 eq) under N₂ atmosphere. The mixture was stirred at 30° C. for 0.5 hr under N₂ atmosphere. Then 4,4-dimethylcyclohex-2-en-1-amine (38.21 mg, 305.20 umol, 1.2 eq) was added. The mixture was stirred at 30° C. for 2 hr under N₂ atmosphere. LCMS showed there was no starting material and main desired compound. The mixture was added in water (10 mL) and stirred for 10 mins. The mixture was extracted with EtOAc (15 mL×3). The mixture was dried with anhydrous Na₂SO₄, filtered and concentrated in vacuo. The crude product was purified by reversed-phase HPLC (column: YMC-Actus Triart C18 150*30 mm*5 um; mobile phase: [water (0.1% TFA)-ACN]; B %: 65%-83%, 9 min). Compound 4-chloro-N-(4,4-dimethylcyclohex-2-en-1-yl)-1H-pyrrolo[2,3-c]pyridine-2-carboxamide (10.6 mg, 34.54 umol, 13.58% yield, 99% purity) was obtained as a white solid.

LCMS (ESI) m/z 304.1 [M+H]⁺; ¹H NMR (400 MHz, DMSO-d6) δ=0.95-1.10 (m, 6H) 1.23 (br s, 1H) 1.42-1.53 (m, 1H) 1.56-1.75 (m, 2H) 1.86 (br d, J=3.91 Hz, 1H) 4.49 (br d, J=5.62 Hz, 1H) 5.47 (dd, J=10.03, 2.45 Hz, 1H) 5.55-5.61 (m, 1H) 7.42 (s, 1H) 8.18 (s, 1H) 8.73 (s, 1H) 8.78 (br d, J=8.07 Hz, 1H) 12.49 (br s, 1H).

### Example 103. MPL-170

### Synthesis of N-(4-bicyclo[2.2.2]octanyl)-4-chloro-1H-pyrrolo[2,3-c]pyridine-2-carboxamide

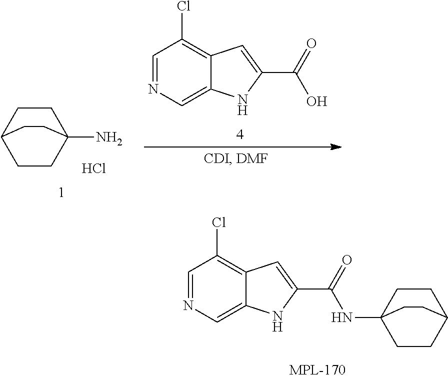 [SMILES: O=C(O)c1cc2c(Cl)cncc2[nH]1 ; NC12CCC(CC1)CC2 ; Cl ; O=C(NC12CCC(CC1)CC2)c1cc2c(Cl)cncc2[nH]1]

To a solution of 4-chloro-1H-pyrrolo[2,3-c]pyridine-2-carboxylic acid (50 mg, 254.34 umol, 1 eq) and bicyclo[2.2.2]octan-4-amine (61.68 mg, 381.50 umol, 1.5 eq, HCl) in DMF (1 mL) was added HOBt (51.55 mg, 381.50 umol, 1.5 eq) and EDCI (73.13 mg, 381.50 umol, 1.5 eq) TEA (77.21 mg, 763.01 umol, 106.20 uL, 3 eq), the mixture was stirred at 25° C. for 4 hr under N₂. LC-MS showed the starting material 1 was consumed completely and one main peak with desired mass was detected. The mixture was added to water (15 mL) and stirred for 10 min, filtered and the filter cake was dried under reduced pressure. The product N-(4-bicyclo[2.2.2]octanyl)-4-chloro-1H-pyrrolo[2,3-c]pyridine-2-carboxamide (54.9 mg, 179.67 umol, 70.64% yield, 99.417% purity) was obtained as white solid.

LCMS (ESI) m/z 304.0 [M+H]⁺; ¹H NMR (500 MHz, METHANOL-d4)=12.34 (br s, 1H), 8.71 (s, 1H), 8.16 (s, 1H), 7.96 (s, 1H), 7.38 (s, 1H), 2.01-1.89 (m, 6H), 1.69-1.60 (m, 6H), 1.58-1.52 (m, 1H).

### Example 104. MPL-174

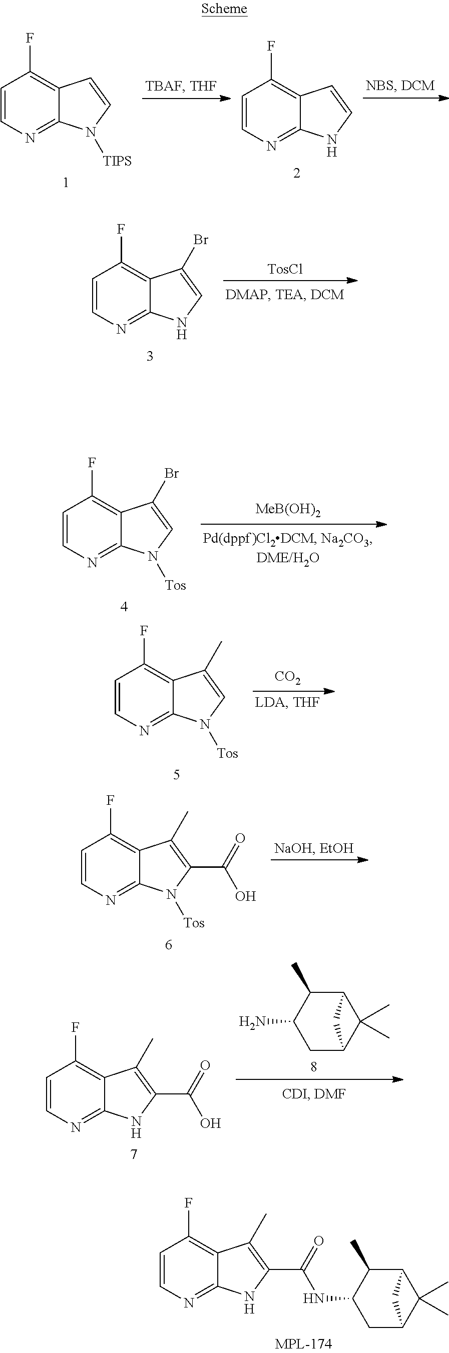

### Synthesis of 3-bromo-4-fluoro-1-(p-tolylsulfonyl)pyrrolo[2,3-b]pyridine

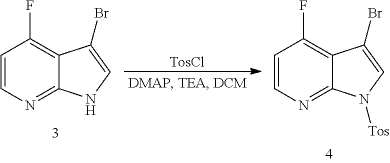

To a solution of 3-bromo-4-fluoro-1H-pyrrolo[2,3-b]pyridine (1.50 g, 6.98 mmol, 1 eq) and NaH (837.12 mg, 20.93 mmol, 60% purity, 3 eq) in THE (15 mL) at 0° C. was added TosCl (1.86 g, 9.77 mmol, 1.4 eq) the mixture was stirred at 25° C. for 12 h. TLC and LCMS showed the starting material 3 was consumed and the desired mass was detected. The reaction mixture was diluted with EtOAc (100 mL) and washed with brine (50 mL×3). The organic layers were dried over anhydrous Na₂SO₄ and concentrated under reduced pressure to give a residue. The residue was purified by column chromatography (SiO₂, Petroleum ether/EtOAc=1:0 to 3:1). The product 3-bromo-4-fluoro-1-(p-tolylsulfonyl)pyrrolo[2,3-b]pyridine (1.8 g, 4.88 mmol, 69.89% yield) was obtained as brown solid.

### Synthesis of 4-fluoro-3-methyl-1-(p-tolylsulfonyl)pyrrolo[2,3-b]pyridine-2-carboxylic acid

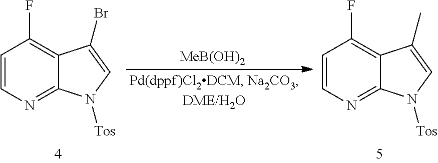

To a solution of 3-bromo-4-fluoro-1-(p-tolylsulfonyl)pyrrolo[2,3-b]pyridine (400 mg, 1.08 mmol, 1 eq) and methylboronic acid (648.52 mg, 10.83 mmol, 10 eq) in DME (4.5 mL) and H2O (0.5 mL) was added Na₂CO₃ (344.49 mg, 3.25 mmol, 3 eq), Pd(dppf)Cl₂.CH₂Cl₂ (88.47 mg, 108.34 umol, 0.1 eq), methylboronic acid (648.52 mg, 10.83 mmol, 10 eq). The mixture was stirred at 80° C. for 12 hr under N₂. LCMS showed the completion of the reaction. The mixture was concentrated under reduce pressure to remove the DME, and diluted with water (100 mL), then extracted with DCM (100 mL×3). The combined organic layers dried over Na₂SO₄, filtered and concentrated under reduced pressure to give a residue. The residue was purified by column chromatography (SiO2, Petroleum ether/EtOAc=1:0 to 1:1). The product 4-fluoro-3-methyl-1-(p-tolylsulfonyl)pyrrolo[2,3-b]pyridine (1.18 g, 1.55 mmol, 143.15% yield, 40% purity) was obtained as white solid.

### Synthesis of 4-fluoro-3-methyl-1-(p-tolylsulfonyl)pyrrolo[2,3-b]pyridine-2-carboxylic acid

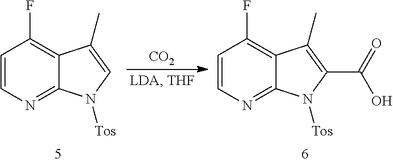

To a solution of 4-fluoro-3-methyl-1-(p-tolylsulfonyl)pyrrolo[2,3-b]pyridine (480 mg, 1.58 mmol, 1 eq) in THE (6 mL) was added LDA (2 M, 1.18 mL, 1.5 eq) at −78° C. under N₂, and the mixture was stirred at the same temperature for 1 h. Then CO₂ (69.41 mg, 1.58 mmol, 1 eq) was added and the mixture was stirred at the same temperature for 0.5 h. LCMS showed the desired product was detected. The reaction was quenched with saturated aqueous NH₄Cl (30 mL) concentrated under reduced pressure to remove the THF. Then acidified with HCl (2 M) to pH=5. The mixture was filtered and the filter cake was washed with 30 mL×3 of Petroleum ether, dried under reduced pressure to give the product. The product 4-fluoro-3-methyl-1-(p-tolylsulfonyl)pyrrolo[2,3-b]pyridine-2-carboxylic acid (500 mg, 1.44 mmol, 91.01% yield) was obtained as white solid.

### Synthesis of 4-fluoro-3-methyl-1H-pyrrolo[2,3-b]pyridine-2-carboxylic acid

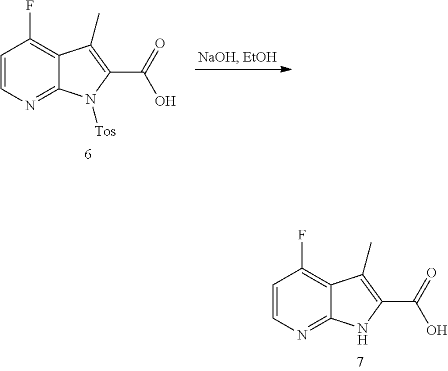

To a solution of 4-fluoro-3-methyl-1-(p-tolylsulfonyl)pyrrolo[2,3-b]pyridine-2-carboxylic acid (500 mg, 1.44 mmol, 1 eq) in THE (3 mL) was added NaOH (2 M, 2.18 mL, 3.04 eq), the mixture was stirred at 75° C. for 2 h. LCMS showed the desired product was detected. The reaction was concentrated under reduced pressure to remove the THF, then acidified with HCl (2 M) to pH=5. The mixture was filtered and the filter cake was washed with 30 mL×3 of Petroleum ether, dried under reduced pressure to give the product. After concentration, the crude product was used directly for the next step without purification. The product 4-fluoro-3-methyl-1H-pyrrolo[2,3-b]pyridine-2-carboxylic acid (220 mg, 453.23 umol, 31.58% yield, 40% purity) was obtained as brown solid.

### Synthesis of 4-fluoro-3-methyl-N-[(1S,2S,3S,5R)-2,6,6-trimethylnorpinan-3-yl]-1H-pyrrolo[2,3-b]pyridine-2-carboxamide

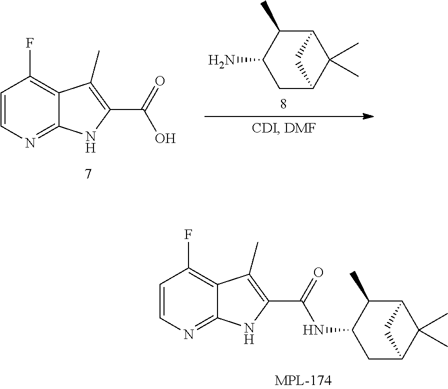 [SMILES: Cc1c(C(=O)O)[nH]c2nccc(F)c12 ; C[C@@H]1[C@@H](N)C[C@H]2C[C@@H]1C2(C)C ; Cc1c(C(=O)N[C@H]2C[C@H]3C[C@@H]([C@@H]2C)C3(C)C)[nH]c2nccc(F)c12]

To a solution of 4-fluoro-3-methyl-1H-pyrrolo[2,3-b]pyridine-2-carboxylic acid (220 mg, 1.13 mmol, 1 eq) in DMF (3 mL) was added CDI (275.59 mg, 1.70 mmol, 1.5 eq). The mixture was stirred at 25° C. for 0.5 h, then (1S,2S,3S,5R)-2,6,6-trimethylnorpinan-3-amine (260.49 mg, 1.70 mmol, 1.5 eq) was added and the mixture was stirred for 0.5 h at the same temperature. LCMS showed the reactant 7 was consumed and the desired mass was detected. The residue was purified by Prep-HPLC (column: YMC-Actus Triart C18 100*30 mm*5 um; mobile phase: [water (0.225% FA)-ACN]; B %: 65%-88%, 11 min) without the further workup. The product 4-fluoro-3-methyl-N-[(1S,2S,3S,5R)-2,6,6-trimethylnorpinan-3-yl]-1H-pyrrolo[2,3-b]pyridine-2-carboxamide (37.6 mg, 113.48 umol, 10.02% yield, 99.417% purity) was obtained as white solid.

LCMS (ESI) m/z 330.2 [M+H]⁺; ¹H NMR (400 MHz, CHLOROFORM-d)=9.94 (br s, 1H), 8.37 (dd, J=5.4, 7.7 Hz, 1H), 6.80 (dd, J=5.4, 10.6 Hz, 1H), 5.95 (br d, J=8.7 Hz, 1H), 4.59-4.45 (m, 1H), 2.82-2.74 (m, 1H), 2.72 (s, 3H), 2.54-2.47 (m, 1H), 2.09-2.01 (m, 1H), 1.98-1.90 (m, 2H), 1.69 (ddd, J=2.4, 6.0, 14.2 Hz, 1H), 1.28 (s, 3H), 1.23 (d, J=7.2 Hz, 3H), 1.11 (s, 3H), 0.94 (d, J=9.9 Hz, 1H).

### Example 105. MPL-187

### Synthesis of 4-chloro-6-oxido-N-[(1S,2S,3S,5R)-2,6,6-trimethylnorpinan-3-yl]-1H-pyrrolo[2,3-c]pyridin-6-ium-2-carboxamide

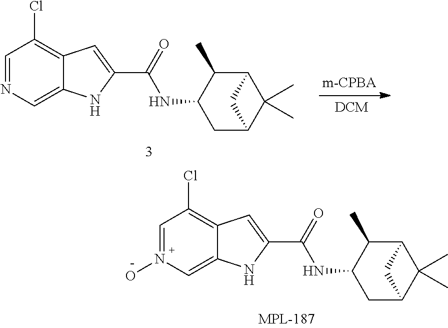

To a solution of 4-chloro-N-[(1S,2S,3S,5R)-2,6,6-trimethylnorpinan-3-yl]-1H-pyrrolo[2,3-c]pyridine-2-carboxamide (90 mg, 271.22 umol, 1 eq) and m-CPBA (175.51 mg, 813.65 umol, 80% purity, 3 eq) in DCM (3 mL). The mixture was stirred at 30° C. for 24 hr. LCMS showed most the starting material was consumed. The mixture was diluted with Na₂SO₃ (10 mL). It was extracted with DCM:MeOH (15 mL×3, 10:1). The organic layers were dried over anhydrous Na₂SO₄ and concentrated under reduced pressure to give a residue. The residue was purified by prep-TLC (SiO₂, DCM:MeOH=10:1). The product 4-chloro-6-oxido-N-[(1S,2S,3S,5R)-2,6,6-trimethylnorpinan-3-yl]-1H-pyrrolo[2,3-c]pyridin-6-ium-2-carboxamide (10.4 mg, 29.90 umol, 11.02% yield, 100% purity) was obtained as white solid. Purity comes from LCMS and the product was confirmed by ¹H NMR.

LCMS (ESI) m/z 348.1 [M+H]⁺; ¹H NMR (400 MHz, DMSO-d6) δ=12.23 (br s, 1H), 8.63 (br d, J=8.3 Hz, 1H), 8.32 (s, 1H), 8.17 (d, J=1.5 Hz, 1H), 7.39 (s, 1H), 4.37 (quin, J=8.0 Hz, 1H), 2.44 (br s, 1H), 2.38 (br d, J=6.8 Hz, 1H), 2.36-2.36 (m, 1H), 2.07 (br t, J=7.2 Hz, 1H), 1.95 (br s, 1H), 1.82 (br t, J=5.4 Hz, 1H), 1.69 (br dd, J=6.4, 11.7 Hz, 1H), 1.23 (s, 4H), 1.19 (d, J=9.5 Hz, 1H), 1.08-1.04 (m, 6H).

### Example 106. MPL-188

### 4-fluoro-N-(1,7,7-trimethylnorbornan-2-yl)-1H-pyrrolo[2,3-c]pyridine-2-carboxamide

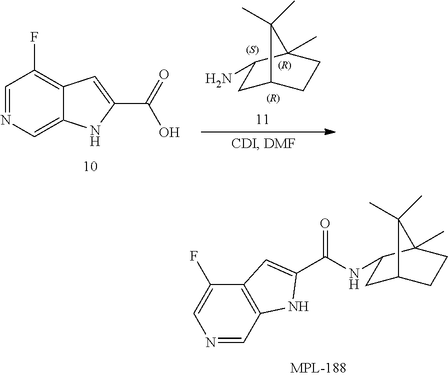

To a solution of 4-fluoro-1H-pyrrolo[2,3-c]pyridine-2-carboxylic acid (40 mg, 222.05 umol, 1 eq) in DMF (1 mL) was added CDI (43.21 mg, 266.47 umol, 1.2 eq). The mixture was stirred at 30° C. for 0.5 h. 1,7,7-trimethylnorbornan-2-amine (40.84 mg, 266.47 umol, 1.2 eq) was added. The mixture was stirred at 30° C. for 11.5 h. LCMS showed the starting material 1 was consumed completely. The reaction mixture was added to water (20 ml), filtered and the filter cake was washed with 10 mL of water, dried in vacuo to give product. The product 4-fluoro-N-(1,7,7-trimethylnorbornan-2-yl)-1H-pyrrolo[2,3-c]pyridine-2-carboxamide (19.9 mg, 63.10 umol, 28.42% yield, 100% purity) was obtained as white solid.

LCMS (ESI) m/z 316.1 [M+H]⁺; ¹H NMR (500 MHz, DMSO-d6) δ=8.65 (d, J=2.7 Hz, 1H), 8.33 (d, J=8.4 Hz, 1H), 8.09 (d, J=2.0 Hz, 1H), 7.51 (s, 1H), 4.43-4.37 (m, 1H), 2.23-2.16 (m, 1H), 1.78 (ddd, J=4.2, 9.2, 13.0 Hz, 1H), 1.71-1.64 (m, 2H), 1.45-1.38 (m, 1H), 1.26 (br t, J=12.7 Hz, 1H), 1.17 (dd, J=4.9, 13.0 Hz, 1H), 0.97 (s, 3H), 0.87 (s, 3H), 0.78 (s, 3H).

### Example 107. MPL-189

### Synthesis of 4-chloro-N-(1,7,7-trimethylnorbornan-2-yl)-1H-pyrrolo[2,3-c]pyridine-2-Carboxamide

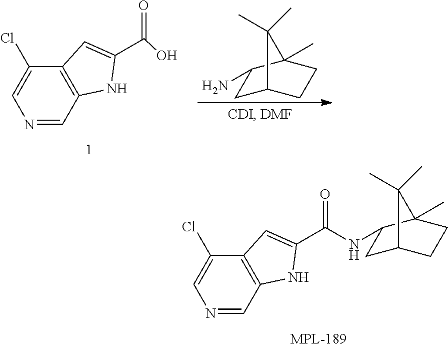 [SMILES: O=C(O)c1cc2c(Cl)cncc2[nH]1 ; CC1(C)C2CCC1(C)C(N)C2 ; CC1(C)C2CCC1(C)C(NC(=O)c1cc3c(Cl)cncc3[nH]1)C2]

To a solution of 4-chloro-1H-pyrrolo[2,3-c]pyridine-2-carboxylic acid (150 mg, 763.01 umol, 1 eq) and CDI (148.46 mg, 915.61 umol, 1.2 eq) in DMF (5 mL). The mixture was stirred at 25° C. for 0.5 h. 1,7,7-trimethylnorbornan-2-amine (140.33 mg, 915.61 umol, 1.2 eq) was added. The mixture was stirred at 25° C. for 11.5 h. LCMS showed no starting material. The reaction mixture was added to water (20 ml), filtered and the filter cake was washed with 10 mL of water, dried in vacuo to give product. The residue was diluted in CH₃CN (5 mL) and H₂O (20 mL) and then lyophilized. The product 4-chloro-N-(1,7,7-trimethylnorbornan-2-yl)-1H-pyrrolo[2,3-c]pyridine-2-carboxamide (196.1 mg, 590.95 umol, 77.45% yield, 100% purity) was obtained as white solid.

LCMS (ESI) m/z 332.2 [M+H]⁺; ¹H NMR (500 MHz, DMSO-d6) δ=12.44 (br s, 1H), 8.73 (s, 1H), 8.42 (br d, J=8.5 Hz, 1H), 8.19 (s, 1H), 7.51 (s, 1H), 4.45-4.37 (m, 1H), 2.24-2.15 (m, 1H), 1.78 (ddd, J=4.1, 9.1, 13.0 Hz, 1H), 1.74-1.65 (m, 2H), 1.43 (dt, J=4.0, 10.3 Hz, 1H), 1.27 (br t, J=11.6 Hz, 1H), 1.19 (dd, J=5.0, 12.9 Hz, 1H), 0.97 (s, 3H), 0.87 (s, 3H), 0.78 (s, 3H).

### Example 108. MPL-191

### N-(1,1-dimethylsilinan-4-yl)-4-fluoro-1H-pyrrolo[2,3-c]pyridine-2-carboxamide

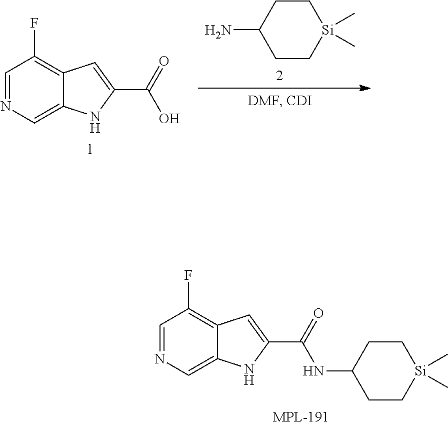

To a solution of 4-fluoro-1H-pyrrolo[2,3-c]pyridine-2-carboxylic acid (200 mg, 1.11 mmol, 1 eq) and CDI (216.04 mg, 1.33 mmol, 1.2 eq) in DMF (2 mL). The mixture was stirred at 25° C. for 0.5 h. 1,1-dimethylsilinan-4-amine (190.93 mg, 1.33 mmol, 1.2 eq) was added. The mixture was stirred at 25° C. for 11.5 h. LCMS showed no starting material. The reaction mixture was added to water (20 mL). Then filtered and the filter cake was washed with 10 mL of water, dried in vacuo to give product. The crude product was purified by re-crystallization from EtOAc (20 mL) at 100° C. to give crude product. The desired product was purified by prep-HPLC (column: YMC-Actus Triart C18 150*30 mm*5 um; mobile phase: [water (0.225% FA)-ACN]; B %: 42%-70%, 11 min). The residue was diluted in CH₃CN (5 mL) and H₂O (20 mL), then lyophilized to give p2. The product N-(1,1-dimethylsilinan-4-yl)-4-fluoro-1H-pyrrolo[2,3-c]pyridine-2-carboxamide (54.9 mg, 177.06 umol, 15.95% yield, 98.5% purity) was obtained as white solid. The product 2 N-(1,1-dimethylsilinan-4-yl)-4-fluoro-1H-pyrrolo[2,3-c]pyridine-2-carboxamide (40 mg, 121.80 umol, 10.97% yield, 93.0% purity) was obtained as white solid.

LCMS (ESI) m/z 306.1 [M+H]⁺; ¹H NMR (500 MHz, DMSO-d₆) δ=12.43 (br s, 1H), 8.64 (d, J=2.4 Hz, 1H), 8.53 (br d, J=8.1 Hz, 1H), 8.08 (d, J=1.5 Hz, 1H), 7.32 (s, 1H), 3.74 (dt, J=8.5, 11.1 Hz, 1H), 2.01 (br d, J=9.5 Hz, 2H), 1.66-1.54 (m, 2H), 0.78 (br d, J=14.5 Hz, 2H), 0.62 (dt, J=4.7, 14.1 Hz, 2H), 0.09 (s, 3H), 0.04 (s, 3H).

### Example 109. MPL-192

### Synthesis of 4-chloro-N-(1,1-dimethylsilinan-4-yl)-1H-pyrrolo[2,3-c]pyridine-2-carboxamide

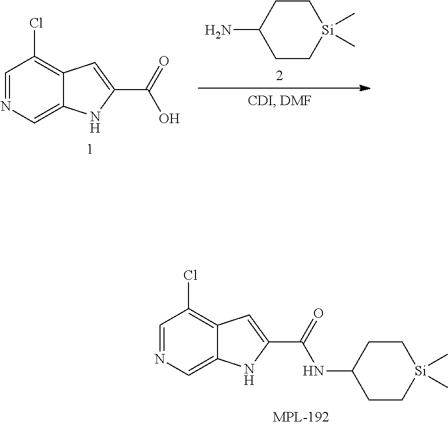

To a solution of 4-chloro-1H-pyrrolo[2,3-c]pyridine-2-carboxylic acid (150 mg, 763.01 umol, 1 eq) in DMF (4 mL) was added CDI (160.84 mg, 991.91 umol, 1.3 eq). The mixture was stirred at 25° C. for 0.5 h. Then 1,1-dimethylsilinan-4-amine (142.14 mg, 991.91 umol, 1.3 eq) was added. The mixture was stirred at 30° C. for 11.5 h. LCMS showed there was no starting material. The reaction was added dropwise to H₂O (20 mL). There was much precipitation which was collected by filter. The cake was diluted with EtOAc (30 mL), dried with anhydrous MgSO₄, filtered. The filtrate was concentrated in vacuo. The residue was diluted in CH₃CN (5 mL) and H₂O (20 mL) lyophilized without further purification. Compound 4-chloro-N-(1,1-dimethylsilinan-4-yl)-1H-pyrrolo[2,3-c]pyridine-2-carboxamide (116.2 mg, 342.06 umol, 44.83% yield, 94.75% purity) was obtained as a white solid.

LCMS (ESI), m/z 322.1[M+H]⁺; ¹H NMR (500 MHz, CHLOROFORM-d) δ=10.78 (br s, 1H), 8.85 (s, 1H), 8.32-8.28 (m, 1H), 6.93 (d, J=1.5 Hz, 1H), 6.28 (br d, J=7.9 Hz, 1H), 4.04-3.95 (m, 1H), 2.28-2.21 (m, 2H), 1.70-1.63 (m, 2H), 1.29-1.25 (m, 1H), 0.89-0.71 (m, 4H), 0.10 (d, J=16.5 Hz, 6H).

### Example 110. MPL-194

### Synthesis of 4-fluoro-6-methyl-N-(1,7, 7-trimethylnorbornan-2-yl)-1H-pyrrolo[2,3-b]pyridine-2-carboxamide

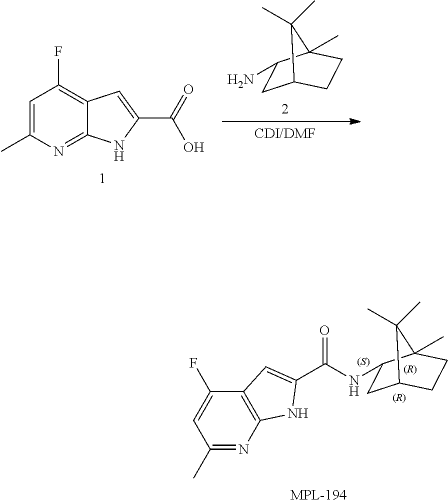

To a solution of 4-fluoro-6-methyl-1H-pyrrolo[2,3-b]pyridine-2-carboxylic acid (100 mg, 515.03 umol, 1 eq) in DMF (0.5 mL) was added CDI (108.57 mg, 669.54 umol, 1.3 eq). The mixture was stirred at 15° C. for 0.5 h. 1,7,7-trimethylnorbornan-2-amine (102.62 mg, 669.54 umol, 1.3 eq) was added and the mixture was stirred at 15° C. for 1.5 h. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. The residue was purified by column chromatography (SiO₂, Petroleum ether:EtOAc=3:1 to 2:1). From LCMS, the product was diluted in CH₃CN (5 mL) and in ultrasound wave for 2 h. There was much precipitation which was collected by filter. The cake was diluted in CH₃CN (5 mL) and H₂O (20 mL), then lyophilized. 4-fluoro-6-methyl-N-(1,7,7-trimethylnorbornan-2-yl)-1H-pyrrolo[2,3-b]pyridine-2-carboxamide (100 mg, 303.57 umol, 1 eq) was diluted in CH₃CN (10 mL) and in ultrasound wave for 2 h. There was much precipitation which was collected by filter. The cake was diluted in CH₃CN (5 mL) and H₂O (20 mL), then lyophilized. Compound 4-fluoro-6-methyl-N-(1,7,7-trimethylnorbornan-2-yl)-1H-pyrrolo[2,3-b]pyridine-2-carboxamide (65 mg, 196.34 umol, 64.68% yield, 99.50% purity) was obtained as a white solid.

LCMS (ESI) m/z 330.1 [M+H]⁺; ¹H NMR (500 MHz, CHLOROFORM-d) 9.57 (br s, 1H), 6.84 (s, 1H), 6.72 (d, J=10.5 Hz, 1H), 6.16 (br d, J=8.5 Hz, 1H), 4.49-4.42 (m, 1H), 2.64-2.61 (m, 3H), 2.50-2.42 (m, 1H), 1.84 (tdd, J=3.9, 8.4, 16.4 Hz, 1H), 1.74 (t, J=4.5 Hz, 1H), 1.58 (ddd, J=4.4, 9.4, 13.9 Hz, 2H), 1.52-1.45 (m, 1H), 1.30-1.23 (m, 1H), 1.00 (s, 3H), 0.92 (s, 3H), 0.89 (s, 3H).

### Example 111. MPL-195

### Synthesis of 4-chloro-6-methyl-N-(1, 7,7-trimethylnorbornan-2-yl)-1H-pyrrolo[2,3-b]pyridine-2-carboxamide

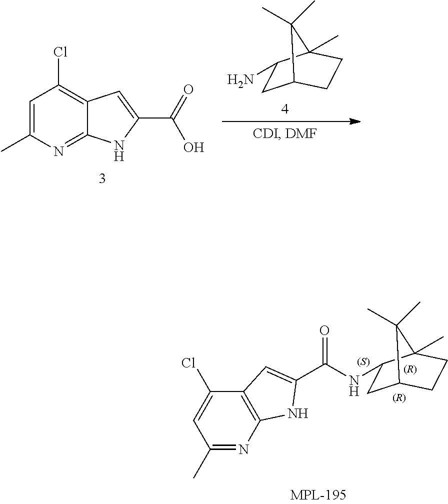

To a solution of 4-chloro-6-methyl-1H-pyrrolo[2,3-b]pyridine-2-carboxylic acid (0.3 g, 1.42 mmol, 1 eq) in DMF (5 mL) (dried by CaH₂) was added CDI (277.16 mg, 1.71 mmol, 1.2 eq). The mixture was stirred at 15° C. for 0.5 h. Then 1,7,7-trimethylnorbornan-2-amine (261.97 mg, 1.71 mmol, 1.2 eq) was added, the mixture was stirred further 12 hr at 30° C. LCMS showed Reactant 3 was consumed completely and one main peak with desired mass was detected. The reaction mixture was dropped into water (30 mL); the product was isolated as white solid. Filtered, the filter cake was washed with water (10 mL×2) to afford the product which was purified by flash silica gel chromatography (ISCO®; 4 g SepaFlash® Silica Flash Column, Eluent of 0-30% EtOAc/Petroleum ether gradient at 18/min). All fractions found to contain product by TLC (Petroleum ether:EtOAc=2:1, Rf=0.5) was combined and concentrated under reduced pressure to give 4-chloro-6-methyl-N-(1,7,7-trimethylnorbornan-2-yl)-1H-pyrrolo[2,3-b]pyridine-2-carboxamide (297 mg, 854.35 umol, 59.98% yield, 99.492% purity) was obtained as a light yellow solid.

LCMS (ESI) m/z 346.1 [M+H]⁺; ¹H NMR (500 MHz, DMSO-d₆) δ=12.29 (s, 1H), 8.14 (d, J=8.61 Hz, 1H), 7.32 (d, J=2.35 Hz, 1H), 7.16 (s, 1H), 4.37 (br s, 1H), 2.51-2.54 (m, 3H), 2.18 (br t, J=11.74 Hz, 1H), 1.72-1.83 (m, 1H), 1.62-1.72 (m, 2H), 1.37-1.45 (m, 1H), 1.21-1.29 (m, 1H), 1.14 (dd, J=12.72, 4.89 Hz, 1H), 0.95 (s, 3H), 0.85 (s, 3H), 0.76 (s, 3H).

### Example 112. MPL-196

### N-(1,1-dimethylsilinan-4-yl)-6-fluoro-4-methoxy-1H-indole-2-carboxamide

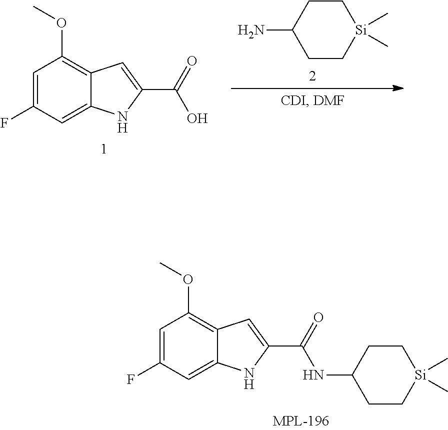

To a solution of 6-fluoro-4-methoxy-1H-indole-2-carboxylic acid (160 mg, 764.91 umol, 1 eq) and CDI (148.84 mg, 917.90 umol, 1.2 eq) in DMF (2 mL). The mixture was stirred at 25° C. for 0.5 h. 1,1-dimethylsilinan-4-amine (131.54 mg, 917.90 umol, 1.2 eq) was added. The mixture was stirred at 25° C. for 11.5 h. LCMS showed no starting material. TLC showed one spot was observed. The reaction mixture was added to water (20 ml), filtered and the filter cake was washed with 10 mL of water, dried in vacuo to give product. The residue was purified by column chromatography (SiO₂, Petroleum ether/EtOAc=1:0 to 10:1). The residue was diluted in CH₃CN (5 mL) and H2O (20 mL), then lyophilized. The product N-(1,1-dimethylsilinan-4-yl)-6-fluoro-4-methoxy-1H-indole-2-carboxamide (141.9 mg, 421.30 umol, 55.08% yield, 99.3% purity) was obtained as white solid.

LCMS (ESI) m/z 335.2 [M+H]⁺; ¹H NMR (500 MHz, DMSO-d6) δ=11.57 (s, 1H), 8.13 (d, J=8.1 Hz, 1H), 7.22 (d, J=1.7 Hz, 1H), 6.71 (dd, J=1.3, 9.5 Hz, 1H), 6.45 (dd, J=1.8, 12.1 Hz, 1H), 3.88 (s, 3H), 3.75-3.64 (m, 1H), 2.00-1.92 (m, 2H), 1.62-1.52 (m, 2H), 0.76 (br d, J=14.5 Hz, 2H), 0.59 (dt, J=4.7, 14.2 Hz, 2H), 0.08 (s, 3H), 0.03 (s, 3H).

### Example 113. MPL-202

### N-(1,1-dimethylsilinan-4-yl)-4-methoxy-1H-pyrrolo[2,3-c]pyridine-2-carboxamide

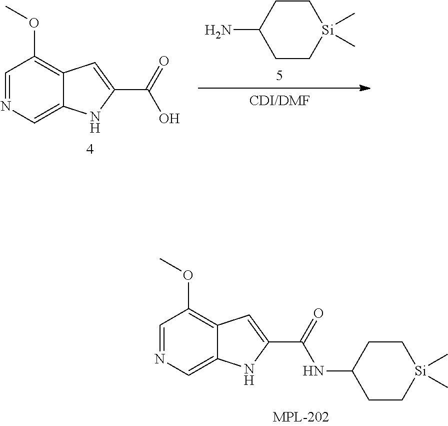

To a solution of 4-methoxy-1H-pyrrolo[2,3-c]pyridine-2-carboxylic acid (200 mg, 1.04 mmol, 1 eq) and CDI (202.51 mg, 1.25 mmol, 1.2 eq) in DMF (2 mL). The mixture was stirred at 25° C. for 0.5 h. 1,1-dimethylsilinan-4-amine (178.97 mg, 1.25 mmol, 1.2 eq) was added. The mixture was stirred at 25° C. for 11.5 h. LCMS showed no starting material. The reaction mixture was added to water (20 mL). Then filtered and the filter cake was washed with 10 mL of water, dried in vacuo to give product. The residue was diluted in CH₃CN (5 mL) and H₂O (20 mL), then lyophilized. The product N-(1,1-dimethylsilinan-4-yl)-4-methoxy-1H-pyrrolo[2,3-c]pyridine-2-carboxamide (137.3 mg, 407.41 umol, 39.15% yield, 94.2% purity) was obtained as yellow solid.

LCMS (ESI) m/z 318.2 [M+H]⁺; ¹H NMR (500 MHz, DMSO-d6) δ=12.02 (br s, 1H), 8.43 (s, 1H), 8.37 (d, J=8.2 Hz, 1H), 7.79 (s, 1H), 7.28 (s, 1H), 3.97 (s, 3H), 3.77-3.67 (m, 1H), 2.05-1.95 (m, 2H), 1.66-1.54 (m, 2H), 0.77 (br d, J=14.5 Hz, 2H), 0.61 (dt, J=4.6, 14.1 Hz, 2H), 0.09 (s, 3H), 0.03 (s, 3H).

### Example 116. MPL-208

### Synthesis of 4-chloro-N-(1,1-dimethylsilinan-4-yl)-6-methyl-1H-pyrrolo[2,3-b]pyridine-2-carboxamide

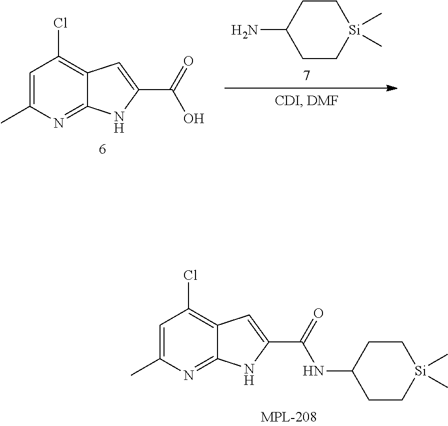

To a solution of 4-chloro-6-methyl-1H-pyrrolo[2,3-b]pyridine-2-carboxylic acid (330 mg, 1.57 mmol, 1 eq) in DMF (5 mL) was added CDI (330.28 mg, 2.04 mmol, 1.3 eq). The mixture was stirred at 10° C. for 0.5 hr. Then 1,1-dimethylsilinan-4-amine (291.89 mg, 2.04 mmol, 1.3 eq) was added. The mixture was stirred at 30° C. further 1 hr. LCMS showed Reactant 1 was consumed completely and one main peak with desired mass was detected. The reaction mixture was dropped into water (50 mL), filtered to afford the crude product which was redissovled in DMF (5 mL), filtered. The filtrate was purified by prep-HPLC (column: Phenomenex Synergi C18 150*30 mm*4 um; mobile phase: [water (0.05% HCl)-ACN]; B %: 53%-83%, 10 min). Compound 4-chloro-N-(1,1-dimethylsilinan-4-yl)-6-methyl-1H-pyrrolo[2,3-b]pyridine-2-carboxamide (110 mg, 327.48 umol, 20.90% yield, 100% purity) was obtained as a white solid.

LCMS (ESI) m/z 336.1 [M+H]⁺; ¹H NMR (500 MHz, DMSO-d₆) δ=12.20 (br s, 1H), 8.26 (d, J=8.09 Hz, 1H), 7.10 (d, J=1.98 Hz, 1H), 7.08 (s, 1H), 3.61 (td, J=11.22, 8.09 Hz, 1H), 2.44 (s, 3H), 1.84-1.94 (m, 2H), 1.45-1.56 (m, 2H), 0.69 (br d, J=14.50 Hz, 2H), 0.52 (td, J=14.08, 4.81 Hz, 2H), 0.00 (s, 3H) −0.05 (s, 3H).

### Example 117. MPL-215

### Synthesis of 4-cyano-N-[(1R,2R,3S,5R)-2-hydroxy-2,6,6-trimethyl-norpinan-3-yl]-1H-pyrrolo[2,3-b]pyridine-2-carboxamide

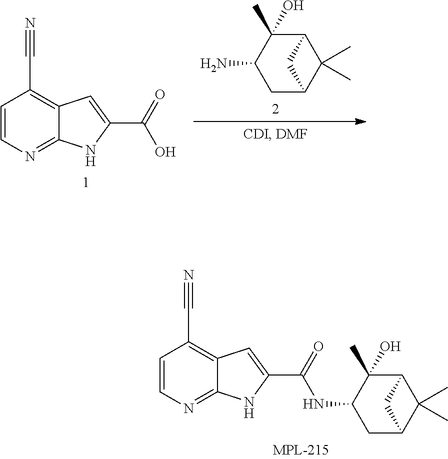 [SMILES: N#Cc1ccnc2[nH]c(C(=O)O)cc12 ; CC1(C)[C@@H]2C[C@H]1[C@@](C)(O)[C@@H](N)C2 ; CC1(C)[C@H]2C[C@H](NC(=O)c3cc4c(C#N)ccnc4[nH]3)[C@](C)(O)[C@@H]1C2]

To a solution of 4-cyano-1H-pyrrolo[2,3-b]pyridine-2-carboxylic acid (100 mg, 534.32 umol, 1 eq) and CDI (112.63 mg, 694.61 umol, 1.3 eq) in DMF (1.5 mL). The mixture was stirred at 30° C. for 0.5 h. Then (1R,2R,3S,5R)-3-amino-2,6,6-trimethyl-norpinan-2-ol (117.57 mg, 694.61 umol, 1.3 eq) was added. The mixture was stirred at 30° C. for 11.5 h. LC-MS showed most of the starting material was consumed. The reaction mixture was added to water (20 mL), then filtered and the filter cake was washed with 10 mL of water, dried in vacuo to give product. The residue was diluted in CH₃CN (5 mL) and H₂O (20 mL), then lyophilized. The product 4-cyano-N-[(1R,2R,3S,5R)-2-hydroxy-2,6,6-trimethyl-norpinan-3-yl]-1H-pyrrolo[2,3-b]pyridine-2-carboxamide (58.3 mg, 159.32 umol, 29.82% yield, 92.478% purity) was obtained as a white solid.

LCMS (ESI) m/z 321.2 [M-OH-]⁺; ¹H NMR (400 MHz, DMSO-d₆) δ=12.89 (br s, 1H), 8.54-8.50 (m, 1H), 8.23 (br d, J=9.0 Hz, 1H), 7.64 (d, J=4.9 Hz, 1H), 7.49 (s, 1H), 4.62-4.49 (m, 2H), 2.27 (br t, J=11.1 Hz, 1H), 2.17-2.09 (m, 1H), 1.90 (br d, J=5.6 Hz, 2H), 1.75-1.61 (m, 2H), 1.27 (s, 3H), 1.23 (s, 3H), 1.07 (s, 3H).

### Example 118. MPL-126

### Synthesis of 5-chloro-N-[(1S,2S,3S,5R)-2,6,6-trimethylnorpinan-3-yl]-1H-pyrrolo[2,3-c]pyridine-2-carboxamide

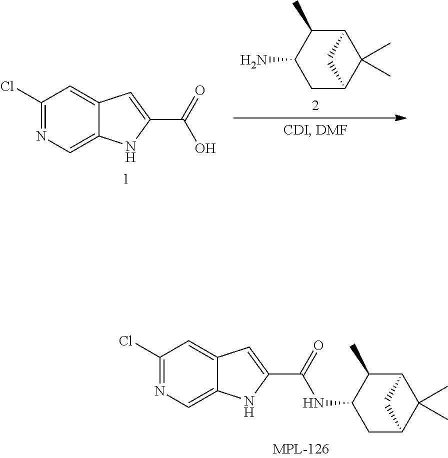 [SMILES: O=C(O)c1cc2cc(Cl)ncc2[nH]1 ; C[C@@H]1[C@@H](N)C[C@H]2C[C@@H]1C2(C)C ; C[C@@H]1[C@@H](NC(=O)c2cc3cc(Cl)ncc3[nH]2)C[C@H]2C[C@@H]1C2(C)C]

To a solution of 5-chloro-1H-pyrrolo[2,3-c]pyridine-2-carboxylic acid (80 mg, 406.94 umol, 1 eq) in DMF (2.0 mL) was added CDI (92.38 mg, 569.71 umol, 1.4 eq) and stirred at 30° C. for 1 h. Then, (1S,2S,3S,5R)-2,6,6-trimethylnorpinan-3-amine (106.03 mg, 691.79 umol, 1.7 eq) was added above solution and stirred at 30° C. for 2 h. LCMS showed the starting material was consumed completely and the desired mass was detected. The mixture was added water (10 mL) and extracted with EtOAc (15 mL×3). The organic phase was washed with water (10 mL×3) and brine (10 mL×3), dried over Na₂SO₄ and filtered and concentrated under reduced pressure to give a residue. The residue was purified by column chromatography (SiO₂, DCM:MeOH=1/0 to 200:1). Compound 5-chloro-N-[(1S,2S,3S,5R)-2,6,6-trimethylnorpinan-3-yl]-1H-pyrrolo[2,3-c]pyridine-2-carboxamide (62.4 mg, 187.50 umol, 46.08% yield, 99.7% purity) was obtained as a white solid.

LCMS (ESI) m/z 332.2 [M+H]⁺; ¹H NMR (500 MHz, DMSO-d₆) δ=12.26 (s, 1H), 8.67 (br d, J=8.4 Hz, 1H), 8.58 (s, 1H), 7.78 (s, 1H), 7.25 (s, 1H), 4.40 (td, J=7.9, 16.4 Hz, 1H), 2.47-2.34 (m, 2H), 2.10 (quin, J=6.9 Hz, 1H), 1.96 (br s, 1H), 1.82 (br t, J=5.6 Hz, 1H), 1.72 (br dd, J=6.4, 12.2 Hz, 1H), 1.26-1.19 (m, 4H), 1.10-1.03 (m, 6H).

### Example 119. MPL-069

### Synthesis of 5-chloro-N-(4,4-dimethylcyclohexyl)-4-fluoro-6-methyl-1H-pyrrolo[2,3-b]pyridine-2-carboxamide

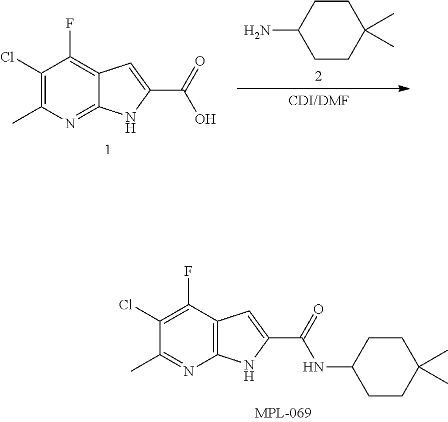

To a solution of 5-chloro-4-fluoro-6-methyl-1H-pyrrolo[2,3-b]pyridine-2-carboxylic acid (80 mg, 349.95 umol, 1 eq) in DMF (1 mL) was added CDI (85.12 mg, 524.92 umol, 1.5 eq). The mixture was stirred at 25° C. for 0.5 h. Then 4,4-dimethylcyclohexanamine (66.78 mg, 524.92 umol, 1.5 eq) was added, the mixture was stirred at 25° C. for 0.5 h. LCMS showed the reaction was consumed and the desired mass was detected. The mixture was purified by prep-HPLC without work up. The residue was purified by prep-HPLC (column: Boston Green ODS 150*30 5u; mobile phase. [water (0.225% FA)-ACN]; B %: 65%-85%, 10 min) to give the white solid (25 mg) and the further purification by SFC (column: DAICEL CHIRALCEL OD-H (250 mm*30 mm, 5 um); mobile phase: [0.1% NH3H2O ETOH]; B %: 25%-25%, min) (SFC (t=7.717 min). The product 5-chloro-N-(4,4-dimethylcyclohexyl)-4-fluoro-6-methyl-1H-pyrrolo[2,3-b]pyridine-2-carboxamide (12.5 mg, 36.95 umol, 10.56% yield, 99.854% purity) was obtained as white solid.

LCMS (ESI) m/z 334.2 [M+H]⁺; ¹H NMR (500 MHz, DMSO-d₆) δ=12.50 (br s, 1H), 8.32 (d, J=8.2 Hz, 1H), 7.22 (s, 1H), 3.79-3.66 (m, 1H), 2.62 (s, 3H), 1.67 (br dd, J=3.5, 12.9 Hz, 2H), 1.58-1.46 (m, 2H), 1.44-1.37 (m, 2H), 1.31-1.23 (m, 2H), 0.93 (d, J=7.8 Hz, 6H).

### Example 120. MPL-207

### Synthesis of N-(1,1-dimethylsilinan-4-yl)-4-fluoro-6-methyl-1H-pyrrolo[2,3-b]pyridine-2-carboxamide

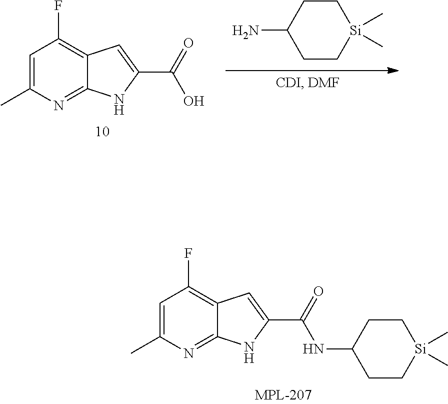

To a solution of 4-fluoro-6-methyl-1H-pyrrolo[2,3-b]pyridine-2-carboxylic acid (150 mg, 772.55 umol, 1 eq) in DMF (0.5 mL) was added CDI (150.32 mg, 927.06 umol, 1.2 eq). The mixture was stirred at 15° C. for 0.5 h. 1,1-dimethylsilinan-4-amine (132.85 mg, 927.06 umol, 1.2 eq) was added and the mixture was stirred at 15° C. for 1.5 h. LCMS showed there were no starting material and main desired compound. The reaction was added dropwise to H₂O (20 mL). There was much precipitation which was collected by filter. The cake was diluted in CH₃CN (5 mL) and H₂O (20 mL), then lyophilized. The crude product was purified by silica column chromatography (eluent of 0-50% EtOAc/Petroleum ether gradient, 4 g silica column). All fractions found to contain product by TLC (Petroleum ether:EtOAc=2:1, Rf=0.3) were combined and evaporated. Compound N-(1,1-dimethylsilinan-4-yl)-4-fluoro-6-methyl-1H-pyrrolo[2,3-b]pyridine-2-carboxamide (110 mg, 341.52 umol, 44.21% yield, 99.18% purity) was obtained as a white solid which was confirmed by LCMS and ¹H NMR.

LCMS (ESI) m/z 320.1 [M+H]⁺; ¹H NMR (400 MHz, CHLOROFORM-d)=9.82 (br s, 1H), 6.74 (s, 1H), 6.67 (d, J=10.5 Hz, 1H), 6.03 (br d, J=8.1 Hz, 1H), 3.91-3.81 (m, 1H), 2.59 (s, 3H), 2.13 (td, J=3.3, 9.2 Hz, 2H), 1.60-1.46 (m, 2H), 0.79-0.61 (m, 4H), 0.04 (s, 3H), 0.00 (s, 3H).

### Example 121. MPL-237

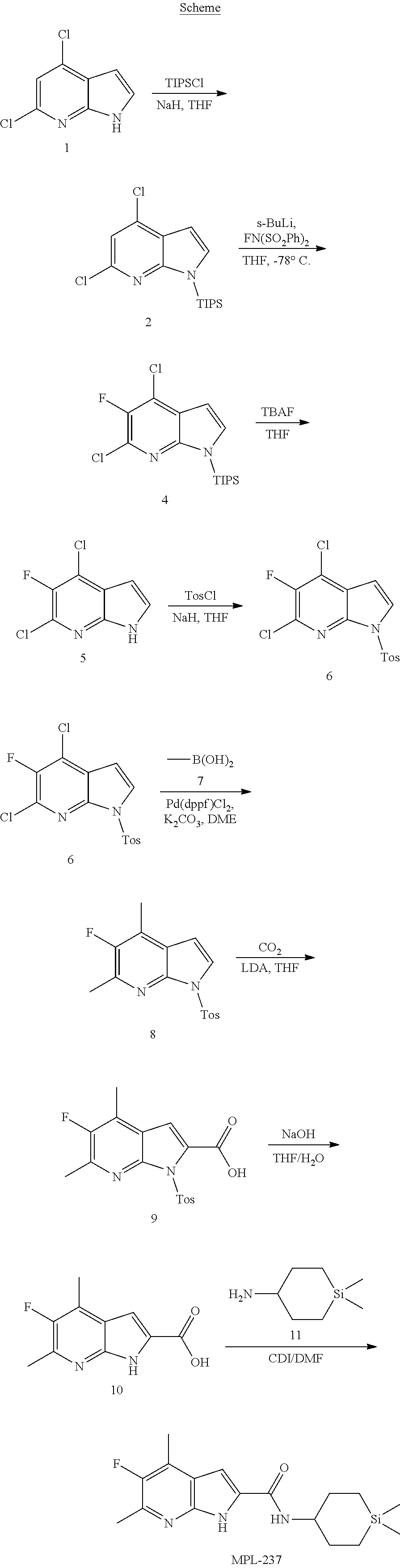

### Synthesis of (4,6-dichloropyrrolo[2,3-b]pyridin-1-yl)-triisopropyl-silane

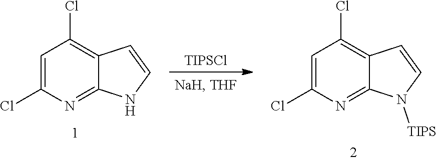

To a solution of NaH (522.92 mg, 13.07 mmol, 60% purity, 3 eq) in 5 mL THF was added a solution of 4,6-dichloro-1H-pyrrolo[2,3-b]pyridine (0.815 g, 4.36 mmol, 1 eq) in 10 mL THF at 0° C. under N₂, then TIPSCl (1.26 g, 6.54 mmol, 1.40 mL, 1.5 eq) was added at 0° C. under N₂. The mixture was stirred at 10° C. for 12 hrs under N₂ atmosphere. TLC (Petroleum ether:EtOAc=1:0) showed there was no starting material. The reaction mixture was quenched by addition saturated aqueous NH₄Cl (10 mL) 0° C., and then extracted with EtOAc (20 mL×3). The combined organic layers were washed with brine (10 mL), dried over anhydrous Na₂SO₄, filtered and concentrated under reduced pressure to give a residue. The residue was purified by column chromatography (SiO₂, Petroleum ether:EtOAc=1:0). The product (4,6-dichloropyrrolo[2,3-b]pyridin-1-yl)-triisopropyl-silane (1.17 g, 3.25 mmol, 74.54% yield, 95% purity) was obtained as white oil.

### Synthesis of (4,6-dichloro-5-fluoro-pyrrolo[2,3-b]pyridin-1-yl)-triisopropyl-silane

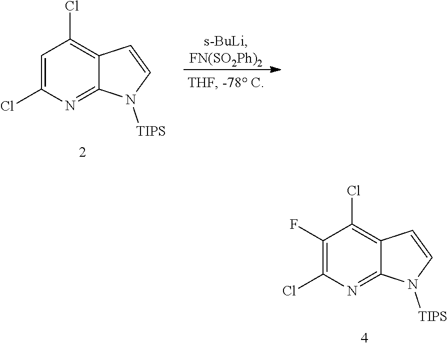

To a solution of (4,6-dichloropyrrolo[2,3-b]pyridin-1-yl)-triisopropyl-silane (1.1 g, 3.20 mmol, 1 eq) in THF (10 mL) at −78° C. under N₂ was treated dropwise with s-BuLi (1.3 M, 5.42 mL, 2.2 eq). The reaction was then stirred for 30 minutes. Then NFSI (2.53 g, 8.01 mmol, 2.5 eq) in THF (20 mL) was added dropwise. The mixture was stirred for 11.5 h at 10° C. under N₂. LCMS showed there was no starting material. The reaction was quenched at 0° C. with saturated aqueous NH₄Cl (20 mL). The aqueous phase was extracted with EtOAc (50 mL×3). The combined hexane phases were dried over anhydrous Na₂SO₄ and concentrated under reduced pressure to give a residue. The residue was purified by column chromatography (SiO₂, Petroleum ether:EtOAc=1:0). (4,6-Dichloro-5-fluoro-pyrrolo[2,3-b]pyridin-1-yl)-triisopropyl-silane (0.942 g, 2.22 mmol, 69.17% yield, 85% purity) was obtained as a white solid.

### Synthesis of 4, 6-dichloro-5-fluoro-1H-pyrrolo[2,3-b]pyridine

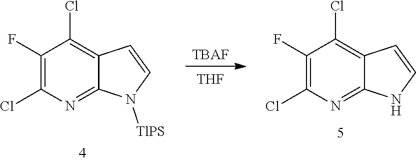

To a solution of (4,6-dichloro-5-fluoro-pyrrolo[2,3-b]pyridin-1-yl)-triisopropyl-silane (0.942 g, 2.61 mmol, 1 eq) in THF (5 mL) was added TBAF (1 M, 3.91 mL, 1.5 eq). The mixture was stirred at 10° C. for 12 hr. TLC (Plate 1: Petroleum ether:EtOAc=1:0) showed there was no starting material. The mixture was concentrated in reduced pressure until without THF. The residue was washed with saturated brine (50 mL). The aqueous phase was extracted with EtOAc (50 mL×3), dried with anhydrous Na₂SO₄, filtered. The filtrate was concentrated in vacuo. The residue was purified by column chromatography (SiO₂, Petroleum ether:EtOAc=1:0 to 3:1). Compound 4,6-dichloro-5-fluoro-1H-pyrrolo[2,3-b]pyridine (629 mg, 2.45 mmol, 94.15% yield, 80% purity) was obtained as a white solid.

### Synthesis of 4,6-dichloro-5-fluoro-1-(p-tolylsulfonyl)pyrrolo[2,3-b]pyridine

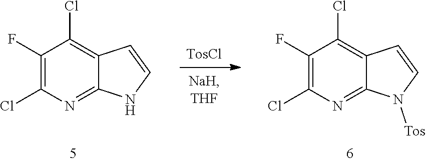

To a solution of 4,6-dichloro-5-fluoro-1H-pyrrolo[2,3-b]pyridine (487 mg, 2.38 mmol, 1 eq) in THE (10 mL) was added NaH (285.05 mg, 7.13 mmol, 60% purity, 3 eq) and 4-methylbenzenesulfonyl chloride (905.74 mg, 4.75 mmol, 2 eq) at 0° C. under N₂. The mixture was stirred at 10° C. for 12 h.

TLC (Petroleum ether:EtOAc=5:1, Rf=0.6) showed there was no starting material and main desired compound. The reaction was added dropwise in saturated aqueous NH₄Cl (10 mL) at 0° C. The aqueous phase was adjusted pH=7 with HCl (2 M). The mixture was concentrated under pressure until without THF. The residue was extracted with EtOAc (10 mL×3). The combined hexane phases were dried over anhydrous Na₂SO₄ and concentrated under reduced pressure to give a residue. The residue was purified by column chromatography (SiO₂, Petroleum ether:EtOAc=1:0 to 10:1). The compound 4,6-dichloro-5-fluoro-1-(p-tolylsulfonyl) pyrrolo[2,3-b]pyridine (802 mg, 1.79 mmol, 75.19% yield, 80% purity) was obtained as a white solid.

### Synthesis of 5-fluoro-4,6-dimethyl-1-(p-tolylsulfonyl)pyrrolo[2,3-b]pyridine

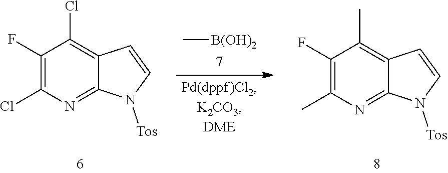

To a solution of 4,6-dichloro-5-fluoro-1-(p-tolylsulfonyl)pyrrolo[2,3-b]pyridine (1 g, 2.23 mmol, 1 eq), methylboronic acid (1.33 g, 22.27 mmol, 10 eq) and K₂CO₃ (923.45 mg, 6.68 mmol, 3 eq) in DME (10 mL) was degassed with N₂ for 3 times. Then Pd(dppf)Cl₂ (162.96 mg, 222.72 umol, 0.1 eq) was added, the mixture was degassed with N₂ for 3 times and stirred at 110° C. for 12 hr under N₂. LCMS showed there was no starting material and main desired compound. The reaction mixture was concentrated under reduced pressure to give a residue. There residue was diluted with EtOAc (50 mL). The mixture was washed with NaCl (50×2 mL). The combine phase was concentrated under reduced pressure. The residue was purified by column chromatography (SiO₂, Petroleum ether:EtOAc=1:0 to 1:1). The compound 5-fluoro-4, 6-dimethyl-1-(p-tolylsulfonyl)pyrrolo[2,3-b]pyridine (406 mg, 1.21 mmol, 54.40% yield, 95% purity) was obtained as a white solid. The compound 5-fluoro-4, 6-dimethyl-1-(p-tolylsulfonyl) pyrrolo[2,3-b]pyridine (436 mg, 1.23 mmol, 55.34% yield, 90% purity) was obtained as white solid.

### Synthesis of 5-fluoro-4, 6-dimethyl-1-(p-tolylsulfonyl) pyrrolo[2, 3-b]pyridine-2-carboxylic acid

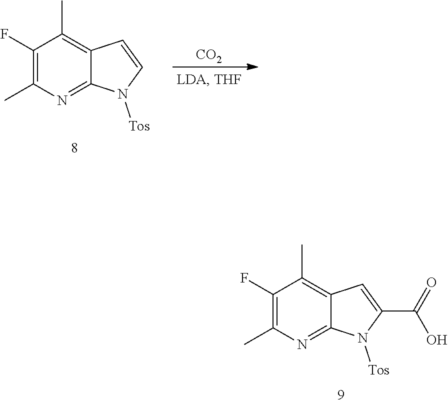

To a solution of 5-fluoro-4,6-dimethyl-1-(p-tolylsulfonyl)pyrrolo[2,3-b]pyridine (231 mg, 725.58 umol, 1 eq) in THF (5 mL) at −78° C. under N₂ was treated dropwise with LDA (2 M, 689.30 uL, 1.9 eq). The reaction was stirred for 1.5 h. The mixture was stirred for 10.5 h at 10° C. under CO₂ (15 Psi). LCMS showed there were main desired compound and a little starting material (2%). The residue was used directly for next step without further work up. The residue was used directly for next step without further purification. The product 5-fluoro-4,6-dimethyl-1-(p-tolylsulfonyl) pyrrolo[2,3-b]pyridine-2-carboxylic acid (262.93 mg, crude) was obtained as a white solid. LCMS (ESI), m/z 363.1[M+H]⁺.

### Synthesis of 5-fluoro-4,6-dimethyl-1H-pyrrolo[2,3-b]pyridine-2-carboxylic acid

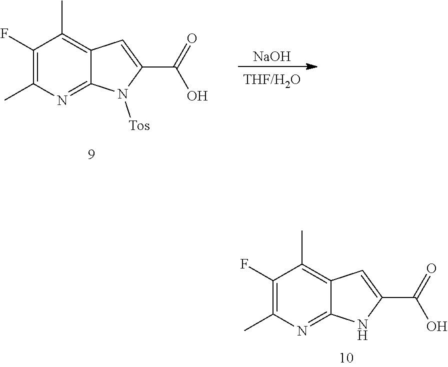

To a solution of 5-fluoro-4, 6-dimethyl-1-(p-tolylsulfonyl) pyrrolo[2, 3-b]pyridine-2-carboxylic acid (262.93 mg, 725.57 umol, 1 eq). The mixture was dropwise added NaOH (4 M, 8 mL, 44.10 eq) until pH=12. The reaction was stirred at 30° C.-70° C. for 2 hr. LCMS showed there was main starting material. The mixture was stirred at 70° C. for 12 hr. LCMS showed there was no starting material and main desired compound. The mixture was concentrated in reduced pressure until without THF. The residue adjusted to pH 4 with HCl (2N), filtered. The cake was transferred the bottom flask. The residue was used directly for next step without further purification. Compound 5-fluoro-4,6-dimethyl-1H-pyrrolo[2,3-b]pyridine-2-carboxylic acid (109 mg, 471.21 umol, 64.94% yield, 90% purity) was obtained as a white solid.

### Synthesis of N-(1,1-dimethylsilinan-4-yl)-5-fluoro-4,6-dimethyl-1H-pyrrolo[2,3-b]pyridine-2-carboxamide

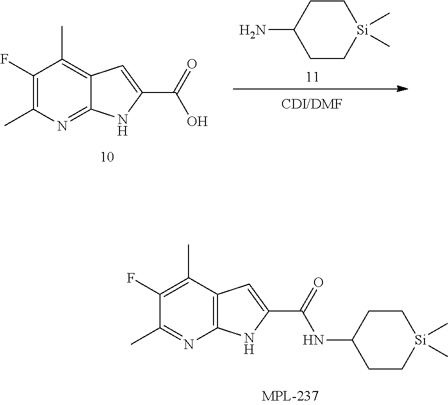

To a solution of 5-fluoro-4,6-dimethyl-1H-pyrrolo[2,3-b]pyridine-2-carboxylic acid (109 mg, 523.56 umol, 1 eq) in DMF (1 mL) was added CDI (110.36 mg, 680.63 umol, 1.3 eq). The mixture was stirred at 30° C. for 0.5 h. 1,1-dimethylsilinan-4-amine (97.54 mg, 680.63 umol, 1.3 eq) was added and the mixture was stirred at 30° C. for 2 h. LCMS showed there were main starting material and desired compound. The reaction was added dropwise to H₂O (20 mL). There was much precipitation which was collected by filter. The cake was diluted in CH₃CN (5 mL) and H₂O (20 mL), then lyophilized. The crude product was purified by preparative HPLC (column: YMC-Actus Triart C18 100*30 mm*5 um; mobile phase: [water (0.225% FA)-ACN]; B %: 60%-85%, 11 min). Compound N-(1,1-dimethylsilinan-4-yl)-5-fluoro-4,6-dimethyl-1H-pyrrolo[2,3-b]pyridine-2-carboxamide (37.4 mg, 110.07 umol, 21.02% yield, 98.14% purity) was obtained as a white solid which was confirmed by LCMS and ¹H NMR.

LCMS (ESI), m/z 334.1[M+H]⁺; ¹H NMR (400 MHz, METHANOL-d₄) 6=7.13 (s, 1H), 3.78 (br t, J=11.0 Hz, 1H), 2.52 (d, J=3.5 Hz, 3H), 2.49 (d, J=2.0 Hz, 3H), 2.13 (br d, J=9.4 Hz, 2H), 1.71-1.60 (m, 2H), 0.87-0.80 (m, 2H), 0.75-0.66 (m, 2H), 0.12 (s, 3H), 0.05 (s, 3H).

### Example 122. MPL-234

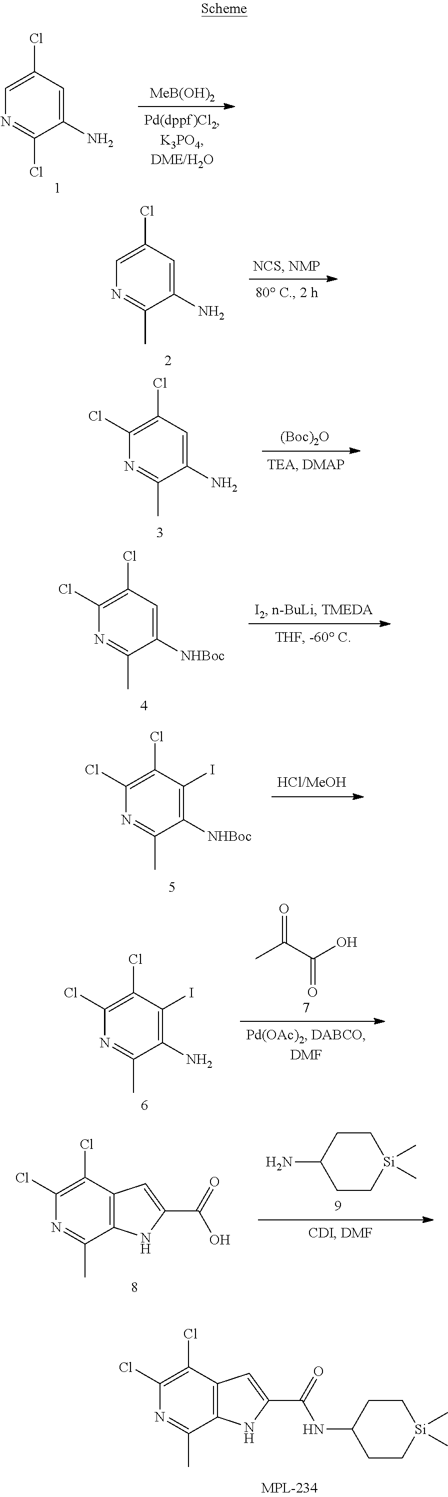

### Synthesis of 5-chloro-2-methyl-pyridin-3-amine

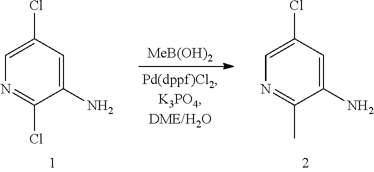

To a solution of 2,5-dichloropyridin-3-amine (5 g, 30.67 mmol, 1 eq), methylboronic acid (2.75 g, 46.01 mmol, 1.5 eq), K₃PO₄ (19.53 g, 92.02 mmol, 3 eq) in DME (45 mL) and H₂O (5 mL) was added Pd(dppf)Cl₂ (2.24 g, 3.07 mmol, 0.1 eq), the mixture was stirred at 120° C. for 12 hr under N₂. LCMS showed the mixture was consumed completely. The mixture was filtered and the filter was concentrated under reduced pressure to give the residue. The residue was purified by column chromatography (SiO₂, Petroleum ether:EtOAc=1:0 to 3:1). The product 5-chloro-2-methyl-pyridin-3-amine (2.3 g, 14.52 mmol, 47.33% yield, 90% purity) was obtained as a green solid (LCMS (ESI) m/z 142.9 [M+H]⁺.

### Synthesis of 5,6-dichloro-2-methyl-pyridin-3-amine

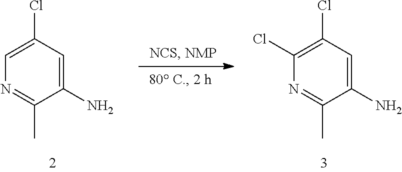

To a solution of 5-chloro-2-methyl-pyridin-3-amine (2.4 g, 16.83 mmol, 1 eq) in NMP (25 mL) was added NCS (2.36 g, 17.67 mmol, 1.05 eq) under N₂, the mixture was stirred for 2 h under 80° C. TLC showed the reactant 2 was consumed completely. The mixture was poured into 200 mL ice-water and extracted with EtOAc (2×200 mL), dried and evaporated. The residue was purified by column chromatography (SiO₂, Petroleum ether:EtOAc=2:1). The product 5,6-dichloro-2-methyl-pyridin-3-amine (2.1 g, 10.68 mmol, 63.43% yield, 90% purity) was obtained as a white solid.

### Synthesis of tert-butyl N-(5,6-dichloro-2-methyl-3-pyridyl)carbamate

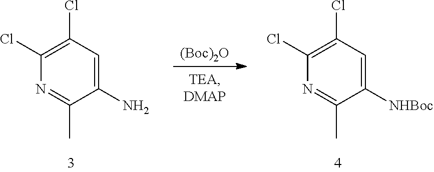

5,6-dichloro-2-methyl-pyridin-3-amine (1 g, 5.65 mmol, 1 eq) in THF (10 mL) was added Boc₂O (1.85 g, 8.47 mmol, 1.95 mL, 1.5 eq). Then TEA (1.71 g, 16.95 mmol, 2.36 mL, 3 eq) and DMAP (138.02 mg, 1.13 mmol, 0.2 eq) was added to above solution and stirred at 10° C. for 12 hr. TLC indicated one major new spot with larger polarity and lower polarity was detected. The reaction was concentrated under reduced pressure to remove solvent. Then the residue was dissolved by EtOAc (20 mL) and washed by H₂O (20 mL) and washed by brine (20 mL). The organic phase was concentrated under reduced pressure. The residue was purified by column chromatography (SiO₂, Petroleum ether:EtOAc=1:0 to 10:1). Compound tert-butyl N-(5,6-dichloro-2-methyl-3-pyridyl)carbamate (600 mg, 1.95 mmol, 34.52% yield, 90% purity) was obtained as a white solid.

### Synthesis of ethyl tert-butyl N-(5,6-dichloro-4-iodo-2-methyl-3-pyridyl)carbamate

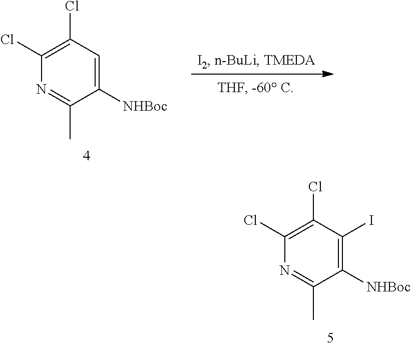 [SMILES: Cc1nc(Cl)c(Cl)cc1NC(=O)OC(C)(C)C ; "I2, n-BuLi, TMEDA" ; Cc1nc(Cl)c(Cl)c(I)c1NC(=O)OC(C)(C)C]

tert-butyl N-(5,6-dichloro-2-methyl-3-pyridyl)carbamate (300 mg, 1.08 mmol, 1 eq) and TMEDA (251.57 mg, 2.16 mmol, 326.72 uL, 2 eq) in THF (5 mL) (dry) was cooled to −60° C. Then n-BuLi (2.5 M, 1.08 mL, 2.5 eq) was added dropwise to above solution at −60° C. and stirred at −60° C. for 1 hr. I₂ (412.10 mg, 1.62 mmol, 327.07 uL, 1.5 eq) in dry THF (5 mL) was added dropwise to above solution at −60° C. and stirred at −60° C. for 1 hr. LCMS showed the desired mass was detected. The reaction was washed by sat. NH₄Cl (10 mL), washed by sat. Na₂SO₃ (10 mL). The mixture was concentrated under reduced pressure to remove solvent. Then the mixture was extracted with EtOAc (10 mL×2). The organic phase was concentrated under reduced pressure. Compound tert-butyl N-(5,6-dichloro-4-iodo-2-methyl-3-pyridyl)carbamate (436 mg, crude) was obtained as yellow oil.

### Synthesis of 5,6-dichloro-4-iodo-2-methyl-pyridin-3-amine

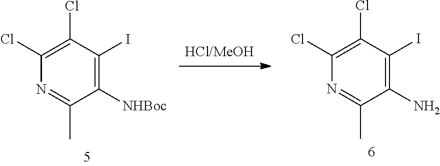 [SMILES: Cc1nc(Cl)c(Cl)c(I)c1NC(=O)OC(C)(C)C ; Cc1nc(Cl)c(Cl)c(I)c1N]

tert-butyl N-(5,6-dichloro-4-iodo-2-methyl-3-pyridyl)carbamate (436 mg, 1.08 mmol, 1 eq) in HCl/MeOH (4 M, 4 mL, 14.79 eq) was stirred at 10° C. for 30 min. LCMS showed 30% desired mass. Then the reaction was added HCl/MeOH (4 M, 2 mL). TLC indicated one major new spot with larger polarity was detected. The reaction was concentrated under reduced pressure. The mixture was washed by sat. Na₂CO₃ (25 mL) and extracted with EtOAc (20 mL×2). The organic phase was concentrated under reduced pressure. The residue was purified by column chromatography (SiO₂, Petroleum ether:EtOAc=1:0 to 3:1). Compound 5,6-dichloro-4-iodo-2-methyl-pyridin-3-amine (113 mg, 354.38 umol, 32.76% yield, 95% purity) was obtained as a yellow solid.

### Synthesis of 4,5-dichloro-7-methyl-1H-pyrrolo[2,3-c]pyridine-2-carboxylic acid

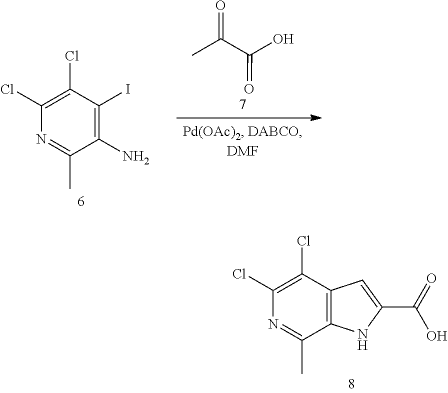

A mixture of 5,6-dichloro-4-iodo-2-methyl-pyridin-3-amine (113 mg, 373.03 umol, 1 eq), 2-oxopropanoic acid (65.70 mg, 746.05 umol, 52.56 uL, 2 eq), DABCO (83.69 mg, 746.05 umol, 82.05 uL, 2 eq) and Pd(OAc)₂ (16.75 mg, 74.61 umol, 0.2 eq) in DMF (3 mL) under N₂ was stirred at 110° C. for 12 hr. LCMS showed the desired product was detected. The mixture was concentrated under reduced pressure to remove solvent. The residue was dissolved with H₂O (5 mL) and neutralized with 6 M HCl to pH=3. Then the precipitate was formed, and the suspension was filtered and the filter cake was washed with water (5 mL), collected and lyophilized. Compound 4,5-dichloro-7-methyl-1H-pyrrolo[2,3-c]pyridine-2-carboxylic acid (90 mg, 348.89 umol, 93.53% yield, 95% purity) was obtained as a brown solid. LCMS (ESI), m/z 244.9[M+H]⁺

### Synthesis of 4,5-dichloro-N-(1,1-dimethylsilinan-4-yl)-7-methyl-1H-pyrrolo[2,3-c]pyridine-2-carboxamide

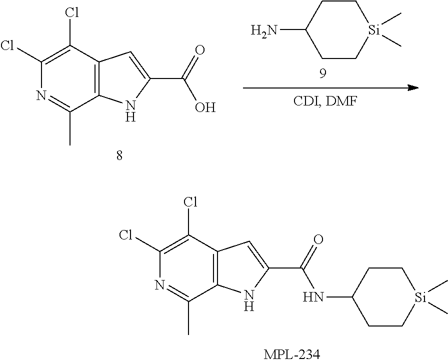

A solution of 4,5-dichloro-7-methyl-1H-pyrrolo[2,3-c]pyridine-2-carboxylic acid (50 mg, 204.03 umol, 1 eq) and CDI (39.70 mg, 244.84 umol, 1.2 eq) in DMF (1.5 mL) was stirred at 30° C. for 0.5 hr. LCMS showed the desired product was detected. 1,1-dimethylsilinan-4-amine (34.96 mg, 243.97 umol, 1.2 eq) was added to above step solution (60 mg, 203.30 umol, 1 eq) in DMF (0.5 mL) was stirred at 30° C. for 1 hr. LCMS showed the desired product was detected. The mixture was not work up and purified by prep-HPLC. The mixture was purified by prep-HPLC (column: YMC-Actus Triart C18 100*30 mm*5 um; mobile phase: [water (0.225% FA)-ACN]; B %: 65%-88%, 11 min). Compound 4,5-dichloro-N-(1,1-dimethylsilinan-4-yl)-7-methyl-1H-pyrrolo[2,3-c]pyridine-2-carboxamide (9 mg, 24.30 umol, 11.95% yield, 100% purity) was obtained as a white solid (LCMS (ESI), m/z 370.0[M+H]⁺).

¹H NMR (500 MHz, DMSO-d6) δ=12.48 (br s, 1H), 8.51 (br d, J=7.9 Hz, 1H), 7.19 (s, 1H), 3.77-3.58 (m, 1H), 2.61 (s, 3H), 1.97-1.82 (m, 2H), 1.67-1.39 (m, 2H), 0.69 (br d, J=14.6 Hz, 2H), 0.53 (dt, J=4.7, 14.1 Hz, 2H), 0.07-0.11 (m, 6H).

### Example 123. MPL-236

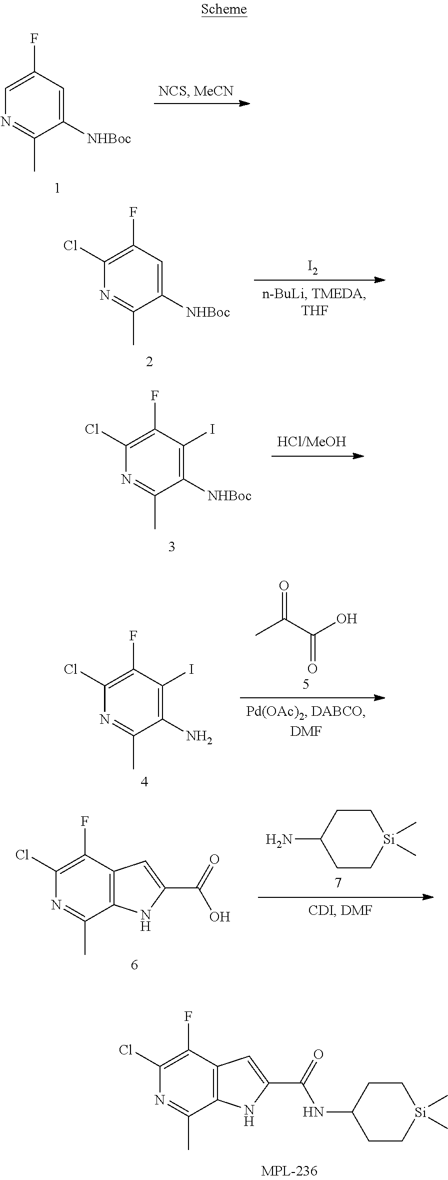

### Synthesis of tert-butyl N-(6-chloro-5-fluoro-2-methyl-3-pyridyl)carbamate

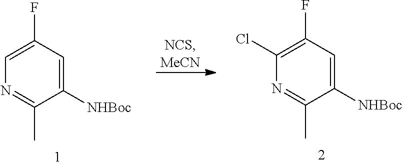

To a solution of tert-butyl N-(5-fluoro-2-methyl-3-pyridyl)carbamate (1 g, 4.42 mmol, 1 eq) in MeCN (10 mL) was added NCS (619.72 mg, 4.64 mmol, 1.05 eq) at 0° C. The mixture was stirred at 65° C. for 16 hr. TLC indicated one major new spot with lower polarity was detected. The reaction mixture was concentrated under reduced pressure to give a residue. The residue was purified by flash silica gel chromatography (ISCO®; 12 g SepaFlash® Silica Flash Column, Eluent of 0-30% EtOAc/Petroleum ether gradient at 30 mL/min). Compound tert-butyl N-(6-chloro-5-fluoro-2-methyl-3-pyridyl)carbamate (600 mg, 2.19 mmol, 49.47% yield, 95% purity) was obtained as a yellow solid.

### Synthesis of tert-butyl N-(6-chloro-5-fluoro-4-iodo-2-methyl-3-pyridyl) carbamate

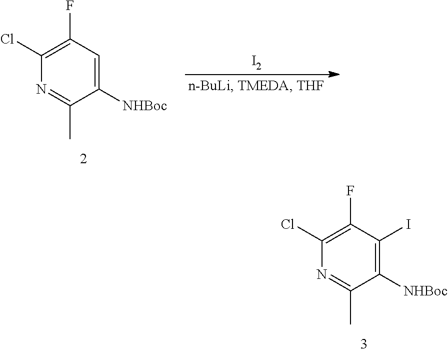 [SMILES: Cc1nc(Cl)c(F)cc1NC(=O)OC(C)(C)C ; "I2" ; Cc1nc(Cl)c(F)c(I)c1NC(=O)OC(C)(C)C]

To a solution of tert-butyl N-(6-chloro-5-fluoro-2-methyl-3-pyridyl)carbamate (600 mg, 2.30 mmol, 1 eq) in THE (10 mL) was added TMEDA (534.91 mg, 4.60 mmol, 694.68 uL, 2 eq). The mixture was added n-BuLi (2.5 M, 1.84 mL, 2 eq) at −78° C. The reaction mixture was stirred at −78° C. for 0.5 hr. Then a solution of I₂ (1.17 g, 4.60 mmol, 927.23 uL, 2 eq) in THE (5 mL) was added. Then reaction mixture was stirred at −78° C. for 1 hr. TLC indicated one major new spot with larger polarity was detected. The reaction mixture was quenched by addition sat. aq. NH₄Cl 15 mL at −78° C., and then diluted with H₂O 20 mL and extracted with EtOAc 60 mL (20 mL×3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0˜25% EtOAc/Petroleum ether gradient at 30 mL/min). Compound tert-butyl N-(6-chloro-5-fluoro-4-iodo-2-methyl-3-pyridyl)carbamate (870 mg, 2.03 mmol, 88.00% yield, 90% purity) was obtained as a yellow solid (LCMS m/z: 386.9[M+H]⁺).

### Synthesis of 6-chloro-5-fluoro-4-iodo-2-methyl-pyridin-3-amine

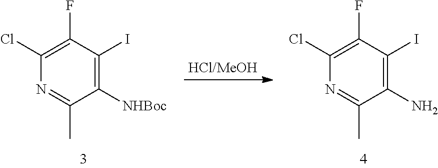 [SMILES: Cc1nc(Cl)c(F)c(I)c1NC(=O)OC(C)(C)C ; Cc1nc(Cl)c(F)c(I)c1N]

A mixture of tert-butyl N-(6-chloro-5-fluoro-4-iodo-2-methyl-3-pyridyl)carbamate (400 mg, 1.03 mmol, 1 eq) in HCl/MeOH (4 M, 10.00 mL, 38.66 eq) was degassed and purged with N₂ for 3 times, and then the mixture was stirred at 30° C. for 4 hr under N₂ atmosphere. TLC indicated one major new spot with larger polarity was detected. The reaction mixture was concentrated under reduced pressure to give a residue. The crude product was used to next step directly. Compound 6-chloro-5-fluoro-4-iodo-2-methyl-pyridin-3-amine (300 mg, crude) was obtained as a yellow solid.

### Synthesis of 5-chloro-4-fluoro-7-methyl-1H-pyrrolo[2,3-c]pyridine-2-carboxylic acid

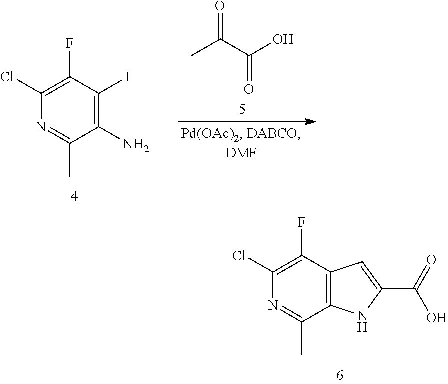

A mixture of 6-chloro-5-fluoro-4-iodo-2-methyl-pyridin-3-amine (300 mg, 1.05 mmol, 1 eq), 2-oxopropanoic acid (184.44 mg, 2.09 mmol, 147.55 uL, 2 eq), Pd(OAc)₂ (47.02 mg, 209.44 umol, 0.2 eq), DABCO (234.94 mg, 2.09 mmol, 230.33 uL, 2 eq) in DMF (8 mL) was degassed and purged with N₂ for 3 times, and then the mixture was stirred at 110° C. for 12 hr under N₂ atmosphere. LC-MS indicated desired mass was detected. The reaction mixture was diluted with H₂O 10 mL and extracted with EtOAc 30 mL (10 mL×3). The combined organic layers were dried over Na₂SO₄, filtered and concentrated under reduced pressure to give a residue. The residue was purified by prep-HPLC (column: Phenomenex Synergi C18 150×30 mm×4 um; mobile phase: [water (0.05% HCl)-ACN]; B %: 28%-48%, 10 min). Compound 5-chloro-4-fluoro-7-methyl-1H-pyrrolo[2,3-c]pyridine-2-carboxylic acid (110 mg, 481.17 umol, 45.95% yield) was obtained as brown solid.

### Synthesis of 5-chloro-N-(1,1-dimethylsilinan-4-yl)-4-fluoro-7-methyl-1H-pyrrolo[2,3-c]pyridine-2-carboxamide

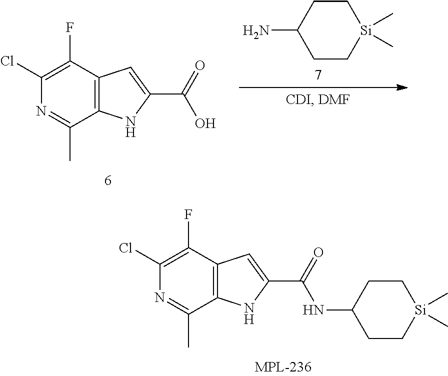

To a solution of 5-chloro-4-fluoro-7-methyl-1H-pyrrolo[2,3-c]pyridine-2-carboxylic acid (50 mg, 218.72 umol, 1 eq) in DMF (1 mL) was added CDI (53.20 mg, 328.07 umol, 1.5 eq). The mixture was stirred at 25° C. for 2 hr. Then the reaction mixture was added 1,1-dimethylsilinan-4-amine (47.01 mg, 328.07 umol, 1.5 eq). The reaction mixture was stirred at 25° C. for 12 hr. LC-MS indicated desired mass was detected. The reaction mixture was drop into water and the product was dissolved out, filtered and dry. The residue was purified by flash silica gel chromatography (ISCO®; 12 g SepaFlash® Silica Flash Column, Eluent of 0˜40% EtOAc/Petroleum ether gradient at 35 mL/min). Compound 5-chloro-N-(1,1-dimethylsilinan-4-yl)-4-fluoro-7-methyl-1H-pyrrolo[2,3-c]pyridine-2-carboxamide (21.1 mg, 58.29 umol, 26.65% yield, 97.759% purity) was obtained as a white solid (LCMS m/z: 354.0 [M+H]⁺).

¹H NMR (400 MHz, METHANOL-d4) 6=7.20 (s, 1H), 3.80 (br t, J=11.2 Hz, 1H), 2.70 (s, 3H), 2.09-2.20 (m, 2H), 1.61-1.74 (m, 2H), 0.80-0.90 (m, 2H), 0.64-0.77 (m, 2H), 0.12 (s, 3H), 0.05 (s, 3H).

### Example 124. MPL-230

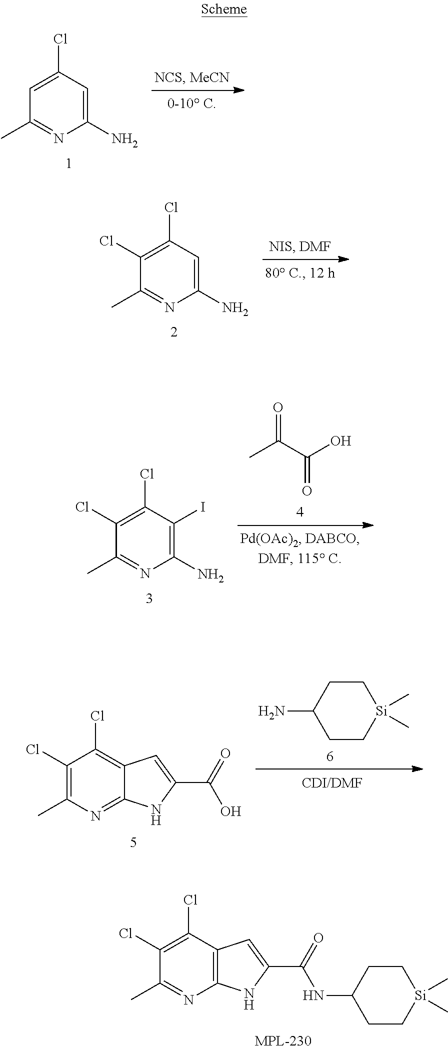

### Synthesis of 4,5-dichloro-6-methyl-pyridin-2-amine

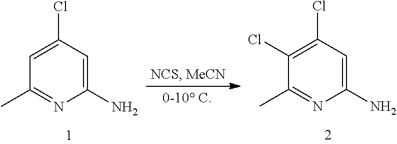

To a solution of 4-chloro-6-methyl-pyridin-2-amine (2.40 g, 16.83 mmol, 1 eq) in MeCN (25 mL) was batch-wise added NCS (2.36 g, 17.67 mmol, 1.05 eq) at −20° C. under N₂, then the temperature was allowed up to 20° C. and the mixture was stirred for 12 h under the same condition. TLC showed the reactant 1 was consumed. The reaction was quenched by addition of water (30 mL). Then extracted with EtOAc (3×50 ml), the organic layers were dried over Na₂SO₄, filtered and concentrated under reduced pressure to give a residue. The residue was purified by column chromatography (SiO₂, Petroleum ether/EtOAc=1:0 to 5:1). The product 4,5-dichloro-6-methyl-pyridin-2-amine (1.4 g, 7.12 mmol, 42.29% yield, 90% purity) was obtained as brown solid.

### Synthesis of 4,5-dichloro-3-iodo-6-methyl-pyridin-2-amine

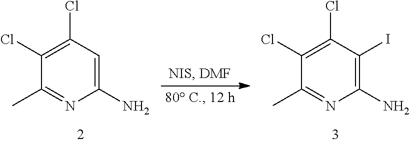

To a solution of 4,5-dichloro-6-methyl-pyridin-2-amine (1.4 g, 7.91 mmol, 1 eq) in DMF (15 mL) was added NIS (3.56 g, 15.82 mmol, 2 eq) under N₂. The mixture was stirred for 12 hr under 80° C. LCMS and TLC showed the reactant 2 was consumed. The mixture was added to water (150 ml) and filtered, the filter cake was solved by EtOAc and the filter was extracted with EtOAc (3×50 mL). The combined organic phase dried and evaporated. The residue was purified by column chromatography (SiO₂, Petroleum ether/EtOAc=1:0 to 5:1). The product 4,5-dichloro-3-iodo-6-methyl-pyridin-2-amine (1.3 g, 3.00 mmol, 37.99% yield, 70% purity) was obtained as orange solid.

### Synthesis of 4,5-dichloro-6-methyl-1H-pyrrolo[2,3-b]pyridine-2-carboxylic acid

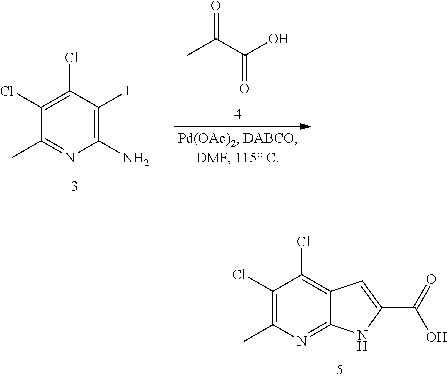

To a solution of 4,5-dichloro-3-iodo-6-methyl-pyridin-2-amine (800 mg, 2.64 mmol, 1 eq) in DMF (15 mL) was added ethyl 2-oxopropanoate (657.10 mg, 3.96 mmol, 625.81 uL, 1.5 eq), DABCO (592.48 mg, 5.28 mmol, 580.86 uL, 2 eq) and Pd(OAc)₂ (118.58 mg, 528.18 umol, 0.2 eq). The mixture was stirred at 115° C. for 4 hr under N₂. LCMS showed the reactant 3 was consumed and the desired mass was detected. The mixture was concentrated under reduced pressure to remove the solvent, then dissolved with NaOH (2M, 20 ml), filtered and the filter was acidified with HCl (6 m) to pH=4, filter to give the crude product. The crude product was used for the next step without the further purification. The crude product 4,5-dichloro-6-methyl-1H-pyrrolo[2,3-b]pyridine-2-carboxylic acid (250 mg, 816.12 umol, 30.90% yield, 80% purity) was obtained as brown solid (LCMS (ESI) m/z 227 [M−H₂O]⁺).

### Synthesis of 4,5-dichloro-N-(1,1-dimethylsilinan-4-yl)-6-methyl-1H-pyrrolo[2,3-b]pyridine-2-carboxamide

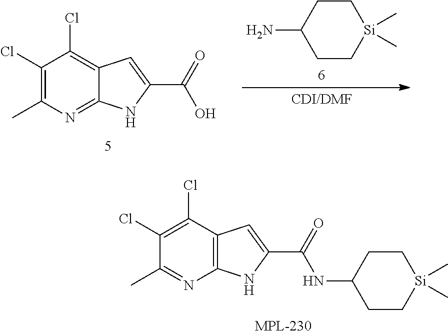

To a solution of 4,5-dichloro-6-methyl-1H-pyrrolo[2,3-b]pyridine-2-carboxylic acid (100 mg, 408.06 umol, 1 eq) in DMF (1.5 mL) was added CDI (99.25 mg, 612.09 umol, 1.5 eq), the mixture was stirred at 30° C. for 0.5 h, then the 1,1-dimethylsilinan-4-amine (87.71 mg, 612.09 umol, 1.5 eq) was added, then the mixture was stirred at 30° C. for 0.5 h. LCMS showed the reactant 5 was consumed completely and the desired mass was detected. The mixture was added to water (15 mL) and stirred for 10 min, filtered and the filter cake was dried under reduced pressure. The crude product was purity by prep-HPLC (column: Phenomenex Synergi C18 150*30 mm*4 um; mobile phase: [water (0.05% HCl)-ACN]; B %: 70%-90%, 10 min). The product 4,5-dichloro-N-(1,1-dimethylsilinan-4-yl)-6-methyl-1H-pyrrolo[2,3-b]pyridine-2-carboxamide (21.6 mg, 57.82 umol, 14.17% yield, 99.137% purity) was obtained as brown solid (LCMS (ESI) m/z 370.0 [M+H]⁺).

¹H NMR (400 MHz, DMSO-d₆) δ=12.49 (s, 1H), 8.39 (d, J=7.8 Hz, 1H), 7.19 (d, J=2.0 Hz, 1H), 3.70 (br s, 1H), 2.63 (s, 3H), 1.96 (br s, 2H), 1.65-1.50 (m, 2H), 0.76 (br d, J=14.9 Hz, 2H), 0.65-0.54 (m, 2H), 0.07 (s, 3H), 0.02 (s, 3H).

### Example 125. MPL-239

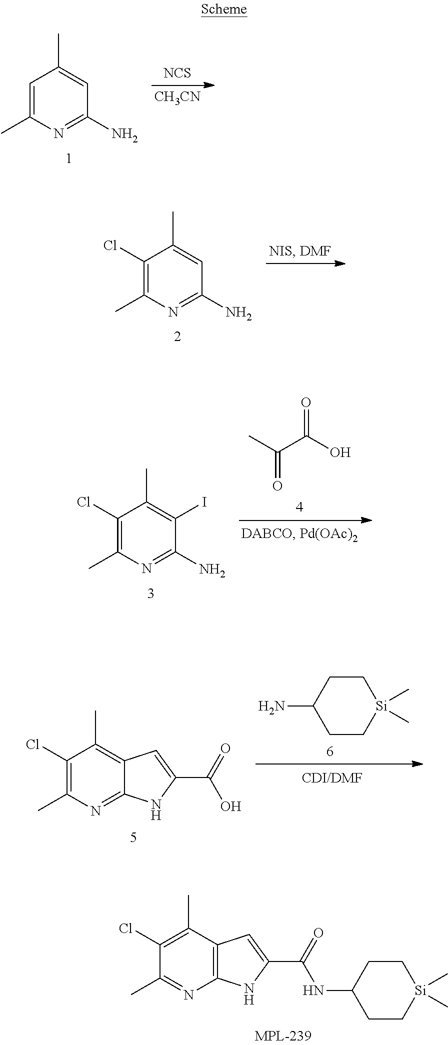

### 5-chloro-4,6-dimethyl-pyridin-2-amine

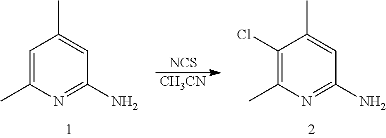

To a solution of 4,6-dimethylpyridin-2-amine (4 g, 32.74 mmol, 1 eq) in CH₃CN (40 mL) was added NCS (4.59 g, 34.38 mmol, 1.05 eq) at 0° C. The mixture was stirred at 10° C. for 12 h. LC-MS showed the starting material was consumed completely. The reaction mixture was concentrated under reduced pressure and diluted with DCM (30 mL) and washed with water (30 mL×3). The organic layers were dried over anhydrous Na₂SO₄ and concentrated under reduced pressure to give a residue. The residue was purified by column chromatography (SiO₂, Petroleum ether:EtOAc=1:0 to 3:1). The product 5-chloro-4,6-dimethyl-pyridin-2-amine (3.3 g, 18.96 mmol, 57.92% yield, 90% purity) was obtained as a yellow solid (LCMS (ESI) m/z 305.1 [M+H]⁺).

### 5-chloro-3-iodo-4,6-dimethyl-pyridin-2-amine

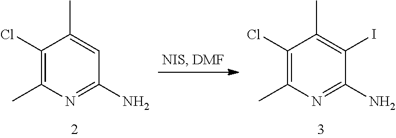

To a solution of 5-chloro-4,6-dimethyl-pyridin-2-amine (3.3 g, 21.07 mmol, 1 eq) in DMF (30 mL) was added NIS (11.85 g, 52.68 mmol, 2.5 eq) at 0° C. Then the mixture was stirred at 10° C. for 12 h. LCMS showed the starting material was remained. NIS (3 g) was added. The mixture was stirred at the same temperature for 12 h. LC-MS showed the starting material was consumed completely. The mixture was concentrated in reduced pressure. Then the mixture was diluted with EtOAc (50 mL). It was washed with aqueous 3% LiCl (50 mL×3). The organic layers were dried over anhydrous Na₂SO₄ and concentrated under reduced pressure to give a residue. The residue was purified by column chromatography (SiO₂, Petroleum ether:EtOAc=1:0 to 10:1). The product 5-chloro-3-iodo-4,6-dimethyl-pyridin-2-amine (2.3 g, 7.73 mmol, 36.71% yield, 95% purity) was obtained as a white solid (LCMS (ESI) m/z 349.1 [M+H]⁺).

### 5-chloro-4,6-dimethyl-1H-pyrrolo[2,3-b]pyridine-2-carboxylic acid

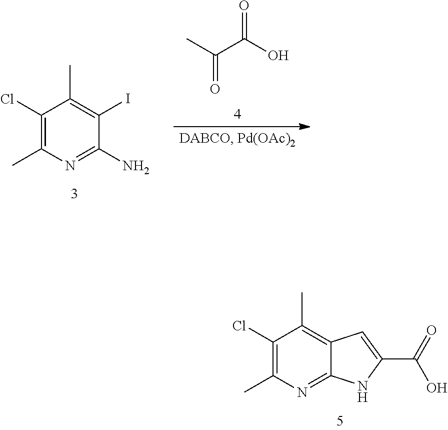

To a solution of 5-chloro-3-iodo-4,6-dimethyl-pyridin-2-amine (1 g, 3.54 mmol, 1 eq), 2-oxopropanoic acid (498.74 mg, 5.66 mmol, 398.99 uL, 1.6 eq) and DABCO (794.12 mg, 7.08 mmol, 778.55 uL, 2 eq) in DMF (15 mL) was added Pd(OAc)₂ (397.35 mg, 1.77 mmol, 0.5 eq) under N₂. The reaction was stirred at 115° C. for 4 h. LC-MS showed the starting material was consumed completely. The reaction mixture was added to water (120 mL), acidified with HCl (2 M) to pH=4. The mixture was filtered and the filter cake was washed with 10 mL×3 of petroleum ether, dried under reduced pressure to give product. The residue was diluted with CH₃CN (5 mL) and H₂O (20 mL), then lyophilized. The product 5-chloro-4,6-dimethyl-1H-pyrrolo[2,3-b]pyridine-2-carboxylic acid (650 mg, 1.45 mmol, 40.87% yield, 50% purity) was obtained as a black solid (LCMS (ESI) m/z 195.0 [M+H]⁺).

### 5-chloro-N-(1,1-dimethylsilinan-4-yl)-4,6-dimethyl-1H-pyrrolo[2,3-b]pyridine-2-carboxamide

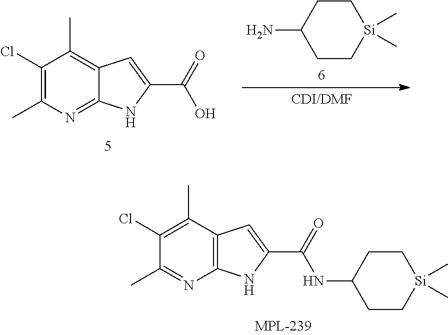

To a solution of 5-chloro-4,6-dimethyl-1H-pyrrolo[2,3-b]pyridine-2-carboxylic acid (100 mg, 445.15 umol, 1 eq) in DMF (1 mL) was added CDI (86.62 mg, 534.18 umol, 1.2 eq). Then the mixture was stirred at 30° C. for 0.5 h. 1,1-dimethylsilinan-4-amine (76.55 mg, 534.18 umol, 1.2 eq) was added. The mixture was stirred at 30° C. for 11.5 h. LC-MS showed the starting material was consumed completely. The reaction mixture was added to water (20 ml), filtered and the filter cake was washed with 10 mL of water, dried in vacuo to give product. The crude product diluted with EtOAc (10 mL). The residue was purified by prep-TLC (SiO₂, Petroleum ether. EtOAc=2:1). The residue was diluted in CH₃CN (1 mL) and H₂O (10 mL), then lyophilized. The product 5-chloro-N-(1,1-dimethylsilinan-4-yl)-4,6-dimethyl-1H-pyrrolo[2,3-b]pyridine-2-carboxamide (19.6 mg, 54.95 umol, 12.34% yield, 98.105% purity) was obtained as a white solid (LCMS (ESI) m/z 350.0 [M+H]⁺).

¹H NMR (500 MHz, DMSO-d₆) δ=12.02 (s, 1H), 8.20 (d, J=8.1 Hz, 1H), 7.17 (d, J=1.8 Hz, 1H), 3.76-3.65 (m, 1H), 2.58 (s, 3H), 2.54 (s, 3H), 2.03-1.95 (m, 2H), 1.64-1.53 (m, 2H), 0.77 (br d, J=14.5 Hz, 2H), 0.61 (dt, J=4.7, 14.1 Hz, 2H), 0.09 (s, 3H), 0.03 (s, 3H).

### Example 126. MPL-253

### Synthesis of 4-fluoro-6-methyl-N-(5-silaspiro[4.5]decan-8-yl)-1H-pyrrolo[2,3-b]pyridine-2-carboxamide

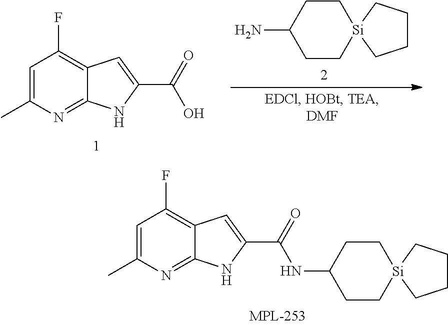 [SMILES: Cc1cc(F)c2cc(C(=O)O)[nH]c2n1 ; NC1CC[Si]2(CCCC2)CC1 ; Cc1cc(F)c2cc(C(=O)NC3CC[Si]4(CCCC4)CC3)[nH]c2n1]

To a solution of 4-fluoro-6-methyl-1H-pyrrolo[2,3-b]pyridine-2-carboxylic acid (100 mg, 515.03 umol, 1 eq) in DMF (1 mL) was added 5-silaspiro[4.5]decan-8-amine (137.79 mg, 669.54 umol, 1.3 eq, HCl). Then a solution of HOBt (208.77 mg, 1.55 mmol, 3 eq) and EDCI (296.20 mg, 1.55 mmol, 3 eq) in DMF (1 mL) was added followed by TEA (156.35 mg, 1.55 mmol, 215.06 uL, 3 eq). The mixture was stirred at 30° C. for 2 hr. LCMS showed there were main starting material and desired compound. The reaction was added dropwise to H₂O (20 mL). There was much precipitation which was collected by filter. The cake was diluted in CH₃CN (5 mL) and H₂O (20 mL), then lyophilized. The residue was purified by prep-HPLC (column: YMC-Actus Triart C18 100*30 mm*5 um; mobile phase: [water (0.225% FA)-ACN]; B %: 60%-88%, 11 min). Compound 4-fluoro-6-methyl-N-(5-silaspiro[4.5]decan-8-yl)-1H-pyrrolo[2,3-b]pyridine-2-carboxamide (30 mg, 85.78 umol, 16.66% yield, 98.79% purity) was obtained as a yellow solid which was confirmed by LCMS and ¹H NMR (LCMS (ESI) m/z 346.1 [M+H]⁺).

¹H NMR (500 MHz, CHLOROFORM-d) δ=9.49 (br s, 1H), 6.78 (s, 1H), 6.73 (d, J=10.5 Hz, 1H), 6.04 (br d, J=7.9 Hz, 1H), 4.00-3.91 (m, 1H), 2.64 (s, 3H), 2.25 (br dd, J=4.6, 7.8 Hz, 2H), 1.66-1.60 (m, 5H), 1.66-1.60 (m, 1H), 0.88-0.79 (m, 4H), 0.63 (br t, J=7.2 Hz, 2H), 0.59 (br t, J=7.3 Hz, 2H).

### Example 127. MPL-100

### Synthesis of 4-(trifluoromethyl)-N-[(1S,2S,3S,5R)-2,6,6-trimethylnorpinan-3-yl]-1H-pyrrolo[2,3-c]pyridine-2-carboxamide

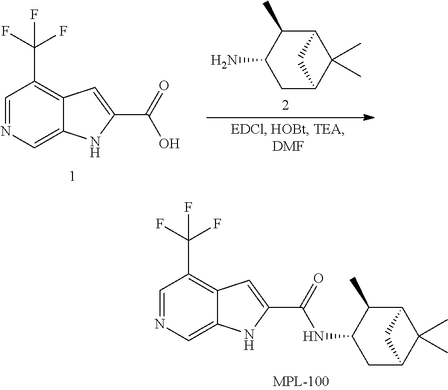 [SMILES: O=C(O)c1cc2c(C(F)(F)F)cncc2[nH]1 ; C[C@@H]1[C@@H](N)C[C@H]2C[C@@H]1C2(C)C ; C[C@@H]1[C@@H](NC(=O)c2cc3c(C(F)(F)F)cncc3[nH]2)C[C@H]2C[C@@H]1C2(C)C]

To a solution of 4-(trifluoromethyl)-1H-pyrrolo[2,3-c]pyridine-2-carboxylic acid (100 mg, 434.51 umol, 1 eq) in DMF (1 mL) was added (1S,2S,3S,5R)-2,6,6-trimethylnorpinan-3-amine (86.57 mg, 564.87 umol, 1.3 eq). Then a solution of HOBt (176.13 mg, 1.30 mmol, 3 eq) and EDCI (249.89 mg, 1.30 mmol, 3 eq) in DMF (1 mL) was added followed by TEA (131.90 mg, 1.30 mmol, 181.44 uL, 3 eq). The mixture was stirred at 30° C. for 2 hr. LCMS showed there were main starting material and desired compound. The reaction was added dropwise to H₂O (20 mL). There was much precipitation which was collected by filter. The cake was diluted with EtOAc (30 mL). It was washed with sat. aq. NaHCO₃ (10 mL×2), aqueous 5% LiCl (10 mL×2), dried with anhydrous Na₂SO₄, filtered and concentrated in vacuo. The residue was delivered without further purification. Compound 4-(trifluoromethyl)-N-[(1S,2S,3S,5R)-2,6,6-trimethylnorpinan-3-yl]-1H-pyrrolo[2,3-c]pyridine-2-carboxamide (92.9 mg, 249.42 umol, 57.40% yield, 98.10% purity) was obtained as a yellow solid which was confirmed by LCMS and ¹H NMR (LCMS (ESI) m/z 366.1 [M+H]⁺).

¹H NMR (500 MHz, DMSO-d₆) δ=9.03 (s, 1H), 8.77 (br s, 1H), 8.50 (br s, 1H), 7.45 (br s, 1H), 4.48-4.39 (m, 1H), 2.17-2.06 (m, 2H), 1.97 (br s, 1H), 1.84 (br s, 1H), 1.74 (br dd, J=6.3, 12.9 Hz, 1H), 1.27-1.21 (m, 5H), 1.10-1.06 (m, 6H).

### Example 128. MPL-254

### Synthesis of 4-fluoro-N-(5-silaspiro[4.5]decan-8-yl)-1H-pyrrolo[2,3-c]pyridine-2-carboxamide

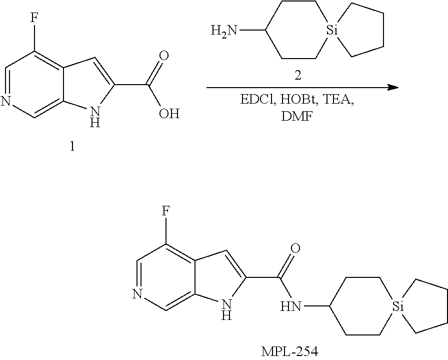 [SMILES: O=C(O)c1cc2c(F)cncc2[nH]1 ; NC1CC[Si]2(CCCC2)CC1 ; O=C(NC1CC[Si]2(CCCC2)CC1)c1cc2c(F)cncc2[nH]1]

To a solution of 4-fluoro-1H-pyrrolo[2,3-c]pyridine-2-carboxylic acid (100 mg, 555.14 umol, 1 eq) in DMF (1 mL) was added 5-silaspiro[4.5]decan-8-amine (125.67 mg, 610.65 umol, 1.1 eq, HCl). Then a solution of HOBt (225.03 mg, 1.67 mmol, 3 eq) and EDCI (319.26 mg, 1.67 mmol, 3 eq) in DMF (1 mL) was added followed by TEA (168.52 mg, 1.67 mmol, 231.81 uL, 3 eq). The mixture was stirred at 30° C. for 2 hr. LCMS showed there were main starting material and desired compound. The reaction was added dropwise to H₂O (20 mL). There was much precipitation which was collected by filter. The cake was diluted in CH₃CN (5 mL) and H₂O (20 mL), then lyophilized. The residue was delivered without further purification. Compound 4-fluoro-N-(5-silaspiro[4.5]decan-8-yl)-1H-pyrrolo[2,3-c]pyridine-2-carboxamide (135.7 mg, 387.58 umol, 69.82% yield, 94.67% purity) was obtained as a gray solid which was confirmed by LCMS and ¹H NMR (LCMS (ESI) m/z 332.1 [M+H]⁺).

¹H NMR (500 MHz, DMSO-d₆) δ=12.43 (br s, 1H), 8.66 (d, J=2.6 Hz, 1H), 8.55 (d, J=8.1 Hz, 1H), 8.09 (d, J=1.8 Hz, 1H), 7.34 (s, 1H), 3.85-3.74 (m, 1H), 2.09 (br d, J=9.0 Hz, 2H), 1.67-1.55 (m, 6H), 0.84-0.73 (m, 4H), 0.63 (br t, J=6.7 Hz, 2H), 0.55 (br t, J=6.7 Hz, 2H).

### Example 129. MPL-259

### Synthesis of 4-fluoro-N-(6-silaspiro[5.5]undecan-3-yl)-1H-pyrrolo[2,3-c]pyridine-2-carboxamide

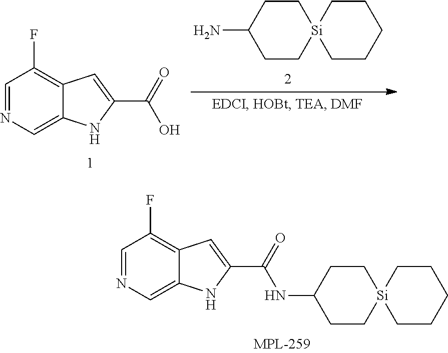 [SMILES: O=C(O)c1cc2c(F)cncc2[nH]1 ; NC1CC[Si]2(CCCCC2)CC1 ; O=C(NC1CC[Si]2(CCCCC2)CC1)c1cc2c(F)cncc2[nH]1]

To a solution of 4-fluoro-1H-pyrrolo[2,3-c]pyridine-2-carboxylic acid (100 mg, 555.14 umol, 1 eq) in DMF (1 mL) was added 6-silaspiro[5.5]undecan-3-amine (134.24 mg, 610.65 umol, 1.1 eq, HCl). Then a solution of HOBt (225.03 mg, 1.67 mmol, 3 eq) and EDCI (319.26 mg, 1.67 mmol, 3 eq) in DMF (1 mL) was added followed by TEA (168.52 mg, 1.67 mmol, 231.81 uL, 3 eq). The mixture was stirred at 30° C. for 2 hr. LCMS showed there were main starting material and desired compound. The reaction was added dropwise to H₂O (20 mL). There was much precipitation which was collected by filter. The cake was diluted with EtOAc (30 mL). It was washed with sta. aq. NaHCO₃ (10 mL×2), aqueous 5% LiCl (10 mL×2), dried with anhydrous Na₂SO₄, filtered and concentrated in vacuo. The residue was delivered without further purification. Compound 4-fluoro-N-(6-silaspiro[5.5]undecan-3-yl)-1H-pyrrolo[2,3-c]pyridine-2-carboxamide (96.6 mg, 270.10 umol, 48.65% yield, 96.60% purity) was obtained as a yellow solid which was confirmed by LCMS and ¹H NMR (LCMS (ESI) m/z 346.1 [M+H]⁺).

¹H NMR (500 MHz, DMSO-d₆) δ=8.65 (d, J=2.6 Hz, 1H), 8.55 (br d, J=8.2 Hz, 1H), 8.09 (d, J=1.7 Hz, 1H), 7.33 (s, 1H), 3.81-3.71 (m, 1H), 2.02 (br d, J=9.3 Hz, 2H), 1.69-1.58 (m, 6H), 1.39 (br s, 2H), 0.92 (br d, J=14.5 Hz, 2H), 0.74-0.68 (m, 2H), 0.64-0.57 (m, 4H).

### Example 130. MPL-190

### Synthesis of 4-(trifluoromethyl)-N-(1,7,7-trimethylnorbornan-2-yl)-1H-pyrrolo[2,3-c]pyridine-2-carboxamide

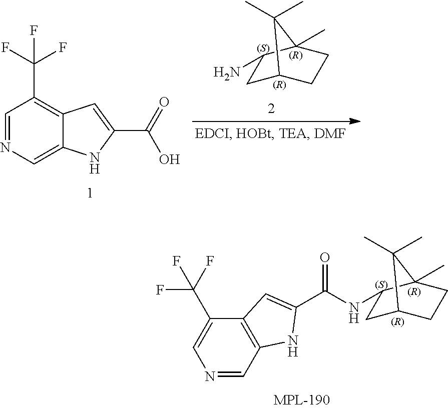

To a solution of 4-(trifluoromethyl)-1H-pyrrolo[2,3-c]pyridine-2-carboxylic acid (100 mg, 434.51 umol, 1 eq) in DMF (1 mL) was added 1,7,7-trimethylnorbornan-2-amine (86.57 mg, 564.86 umol, 1.3 eq). Then a solution of HOBt (176.13 mg, 1.30 mmol, 3 eq) and EDCI (249.89 mg, 1.30 mmol, 3 eq) in DMF (1 mL) was added followed by TEA (131.90 mg, 1.30 mmol, 181.44 uL, 3 eq). The mixture was stirred at 30° C. for 2 hr. LCMS showed there were main starting material and desired compound. The reaction was added dropwise to H₂O (20 mL). There was much precipitation which was collected by filter. The cake was diluted with EtOAc (30 mL). It was washed with sat. aq. NaHCO₃ (10 mL×2), aqueous 5% LiCl (10 mL×2), dried with anhydrous Na₂SO₄, filtered and concentrated in vacuo. The residue was delivered without further purification. Compound 4-(trifluoromethyl)-N-(1,7,7-trimethylnorbornan-2-yl)-1H-pyrrolo[2,3-c]pyridine-2-carboxamide (62.3 mg, 166.68 umol, 38.36% yield, 97.76% purity) was obtained as a white solid which was confirmed by LCMS and ¹H NMR (LCMS (ESI) m/z 366.1 [M+H]⁺).

¹H NMR (400 MHz, DMSO-d₆) δ=12.62 (br s, 1H), 9.00 (s, 1H), 8.50-8.42 (m, 2H), 7.51 (br s, 1H), 4.42 (br s, 1H), 2.21 (br t, J=11.7 Hz, 1H), 1.80-1.66 (m, 3H), 1.47-1.38 (m, 1H), 1.31-1.23 (m, 1H), 1.18 (dd, J=4.9, 13.1 Hz, 1H), 0.97 (s, 3H), 0.87 (s, 3H), 0.78 (s, 3H).

### Example 131. MPL-229

### Synthesis of N-(1, 1-dimethylsilinan-4-yl)-4, 5-difluoro-6-methyl-1H-pyrrolo[2, 3-b]pyridine-2-carboxamide

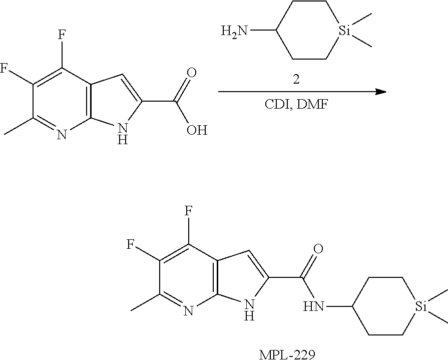

To a solution of 4, 5-difluoro-6-methyl-1H-pyrrolo[2, 3-b]pyridine-2-carboxylic acid (40 mg, 188.54 umol, 1 eq) in DMF (1.5 mL) was added CDI (33.63 mg, 207.40 umol, 1.1 eq). The mixture was stirred at 30° C. for 0.5 h. Then 1, 1-dimethylsilinan-4-amine (29.72 mg, 207.40 umol, 1.1 eq) was added. The mixture was stirred at 30° C. for 11.5 h. LCMS showed there were main desired compound and a little starting material. The reaction was added dropwise to H₂O (20 mL). There was much precipitation which was collected by filter. The cake was transferred in bottom flask. The crude product was purified by prep-TLC (SiO₂, Petroleum ether:EtOAc=5:1). Compound N-(1, 1-dimethylsilinan-4-yl)-4, 5-difluoro-6-methyl-1H-pyrrolo[2, 3-b]pyridine-2-carboxamide (30 mg, 88.39 umol, 46.88% yield, 99.423% purity) was obtained as a white solid (LCMS (ESI), m/z 338.0[M+H]⁺).

¹H NMR (400 MHz, CHLOROFORM-d) δ=9.55 (br s, 1H), 6.80 (s, 1H), 6.04 (br d, J=8.2 Hz, 1H), 3.91 (br d, J=8.2 Hz, 1H), 2.63 (d, J=3.1 Hz, 3H), 2.18 (br d, J=10.2 Hz, 2H), 1.59-1.53 (m, 2H), 0.83-0.68 (m, 4H), 0.10 (s, 3H), 0.06 (s, 3H).

### Example 132. MPL-260

### Synthesis of 4-chloro-N-(6-silaspiro[5.5]undecan-3-yl)-1H-pyrrolo[2,3-c]pyridine-2-carboxamide

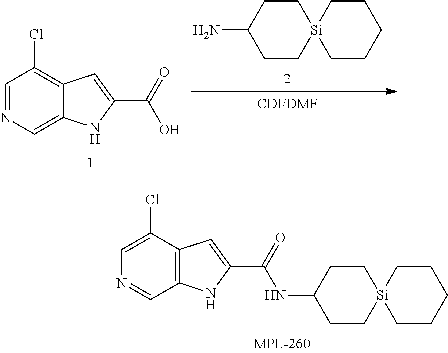 [SMILES: O=C(O)c1cc2c(Cl)cncc2[nH]1 ; NC1CC[Si]2(CCCCC2)CC1 ; O=C(NC1CC[Si]2(CCCCC2)CC1)c1cc2c(Cl)cncc2[nH]1]

To a solution of 4-chloro-1H-pyrrolo[2, 3-c]pyridine-2-carboxylic acid (50 mg, 254.34 umol, 1 eq) in DMF (1.5 mL) was added CDI (49.49 mg, 305.20 umol, 1.2 eq). The mixture was stirred at 30° C. for 0.5 h. Then 6-silaspiro[5.5]undecan-3-amine (55.96 mg, 305.20 umol, 1.2 eq) was added. The mixture was stirred at 30° C. for 11.5 h. TLC (Petroleum ether:EtOAc=5:1, Rf=0.5) showed there were no starting material and one major new spot with higher polarity was detected. The reaction was added dropwise to H₂O (20 mL). The precipitation was collected by filter. The cake was transferred in bottom flask. The residue was purified by column chromatography (SiO₂, Petroleum ether:EtOAc=5:1). Compound 4-chloro-N-(6-silaspiro[5.5]undecan-3-yl)-1H-pyrrolo[2,3-c]pyridine-2-carboxamide (8.6 mg, 23.73 umol, 9.33% yield, 99.880% purity) was obtained as a yellow solid (LCMS (ESI), m/z 362.0 [M+H]⁺).

1H NMR (500 MHz, CHLOROFORM-d) δ=10.82 (br s, 1H), 8.85 (s, 1H), 8.29 (s, 1H), 6.93 (s, 1H), 6.29 (br d, J=7.3 Hz, 1H), 4.01 (br d, J=8.2 Hz, 1H), 2.25 (br d, J=9.5 Hz, 2H), 1.76-1.64 (m, 6H), 1.43 (br s, 2H), 0.95 (br d, J=15.0 Hz, 2H), 0.79-0.69 (m, 4H), 0.68-0.62 (m, 2H).

### Example 133. MPL-209

### Synthesis of N-(1,1-dimethylsilinan-4-yl)-4-fluoro-3,6-dimethyl-1H-pyrrolo[2,3-b]pyridine-2-carboxamide

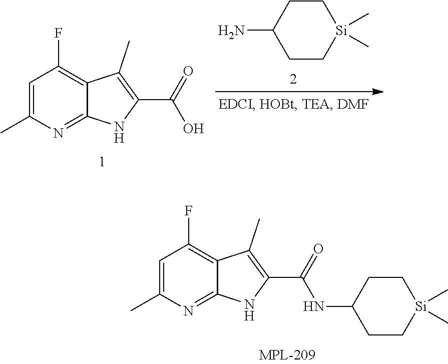

To a solution of 4-fluoro-3,6-dimethyl-1H-pyrrolo[2,3-b]pyridine-2-carboxylic acid (60 mg, 288.20 umol, 1 eq) in DMF (0.5 mL) was added 1,1-dimethylsilinan-4-amine (53.69 mg, 374.66 umol, 1.3 eq). Then a solution of HOBt (116.82 mg, 864.60 umol, 3 eq) and EDCI (165.74 mg, 864.60 umol, 3 eq) in DMF (0.5 mL) was added followed by TEA (87.49 mg, 864.60 umol, 120.34 uL, 3 eq). The mixture was stirred at 30° C. for 2 hr. LCMS showed there were main starting material and desired compound. The reaction was added dropwise to H₂O (20 mL). There was much precipitation which was collected by filter. The cake was diluted in CH₃CN (5 mL) and H₂O (20 mL), then lyophilized. The crude product was purified by silica column chromatography (eluent of 0˜30% EtOAc/Petroleum ether gradient, 4 g silica column). All fractions found to contain the product by TLC (Petroleum ether:EtOAc=3:1, R_{f}=0.3) were combined and evaporated. Compound N-(1,1-dimethylsilinan-4-yl)-4-fluoro-3,6-dimethyl-1H-pyrrolo[2,3-b]pyridine-2-carboxamide (30 mg, 86.66 umol, 30.07% yield, 96.33% purity) was obtained as a white solid which was confirmed by LCMS and ¹H NMR (LCMS (ESI) m/z 334.1 [M+H]⁺).

¹H NMR (400 MHz, METHANOL-d₄) 6=6.75 (d, J=11.3 Hz, 1H), 3.78 (br t, J=11.3 Hz, 1H), 2.62 (s, 3H), 2.56 (s, 3H), 2.17 (br d, J=12.9 Hz, 2H), 1.71-1.60 (m, 2H), 0.88-0.80 (m, 2H), 0.76-0.67 (m, 2H), 0.12 (s, 3H), 0.05 (s, 3H).

### Example 134. MPL-220

### Synthesis of 5-chloro-4-fluoro-N-[(1R,2R,3S,5R)-2-hydroxy-2,6,6-trimethyl-norpinan-3-yl]-6-methyl-1H-pyrrolo[2,3-b]pyridine-2-carboxamide

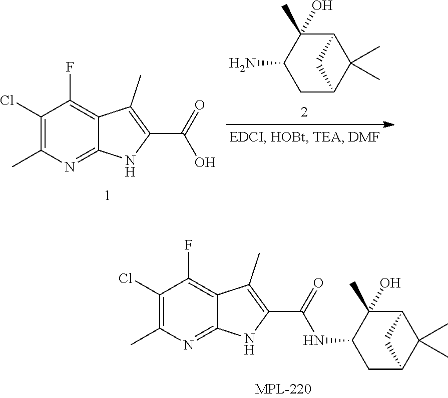

To a solution of 5-chloro-4-fluoro-6-methyl-1H-pyrrolo[2,3-b]pyridine-2-carboxylic acid (120 mg, 524.92 umol, 1 eq) in DMF (1 mL) was added (1R,2R,3S,5R)-3-amino-2,6,6-trimethyl-norpinan-2-ol (115.50 mg, 682.39 umol, 1.3 eq). Then a solution of HOBt (212.78 mg, 1.57 mmol, 3 eq) and EDCI (301.88 mg, 1.57 mmol, 3 eq) in DMF (1 mL) was added followed by TEA (159.35 mg, 1.57 mmol, 219.19 uL, 3 eq). The mixture was stirred at 30° C. for 2 hr. LCMS showed there were starting material and desired compound. The reaction was added dropwise to H₂O (20 mL). There was much precipitation which was collected by filter. The cake was purified by prep-HPLC (column: YMC-Actus Triart C18 100*30 mm*5 um; mobile phase: [water (0.225% FA)-ACN]; B %: 51%-80%, 11 min). Compound 5-chloro-4-fluoro-N-[(1R,2R,3S,5R)-2-hydroxy-2,6,6-tri methyl-norpinan-3-yl]-6-methyl-1H-pyrrolo[2,3-b]pyridine-2-carboxamide (11 mg, 28.74 umol, 5.48% yield, 99.25% purity) was obtained as a yellow solid which was confirmed by LCMS and ¹H NMR (LCMS (ESI) m/z 380.1 [M+H]⁺). ¹H NMR (500 MHz, CHLOROFORM-d) δ=9.57 (br s, 1H), 7.30 (br d, J=7.5 Hz, 1H), 6.88 (d, J=2.1 Hz, 1H), 4.55-4.49 (m, 1H), 2.72 (s, 3H), 2.72-2.66 (m, 1H), 2.32-2.27 (m, 1H), 2.08-2.02 (m, 2H), 1.64 (dt, J=2.0, 6.9 Hz, 1H), 1.45 (d, J=10.4 Hz, 1H), 1.38 (s, 3H), 1.33 (s, 3H), 1.12 (s, 3H).

### Example 135. MPL-232

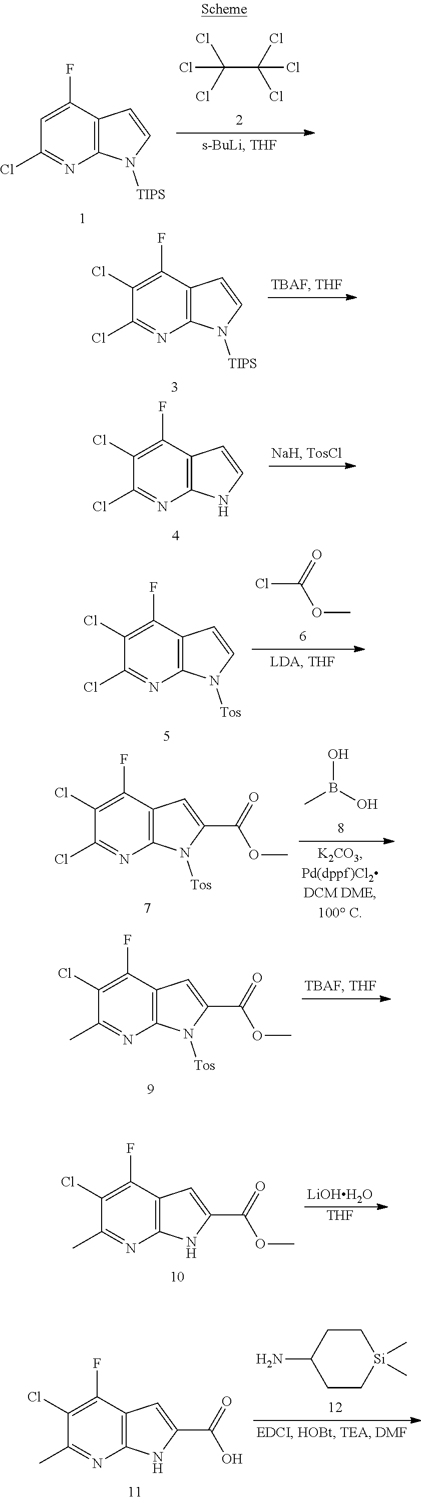

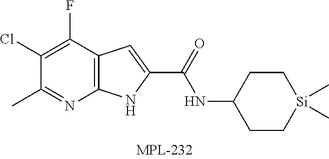

### Synthesis of (5,6-dichloro-4-fluoro-pyrrolo[2,3-b]pyridin-1-yl)-triisopropyl-silane

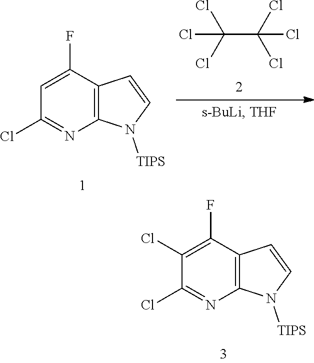

A mixture of (6-chloro-4-fluoro-pyrrolo[2,3-b]pyridin-1-yl)-triisopropyl-silane (10.72 g, 32.79 mmol, 1 eq) in THF (100 mL) was degassed and purged with N₂ for 3 times. s-BuLi (1.3 M in n-hexane, 47.93 mL, 1.9 eq) was added stirred at −60° C. and the reaction was stirred at −60° C. for 30 min under N₂ atmosphere. Then a solution of 1,1,1,2,2,2-hexachloroethane (11.64 g, 49.19 mmol, 5.57 mL, 1.5 eq) in THF (20 mL) was added, and the mixture was stirred −60° C. for 30 min. LC-MS showed desired mass. The reaction mixture was quenched with saturated NH₄Cl solution (20 mL) at 25° C., and then diluted with water (50 mL) and extracted with petroleum ether (100 mL×2). The combined organic layer dried over Na₂SO₄, filtered and concentrated under reduced pressure to give a residue which was purified by column chromatography (SiO₂, 0-20% ethyl acetate in petroleum ether) to afford (5,6-dichloro-4-fluoro-pyrrolo[2,3-b]pyridin-1-yl)-triisopropyl-silane (9.14 g, 20.24 mmol, 61.73% yield, 80% purity) as a yellow oil. ¹H NMR was recorded.

### Synthesis of 5,6-dichloro-4-fluoro-1H-pyrrolo[2,3-b]pyridine

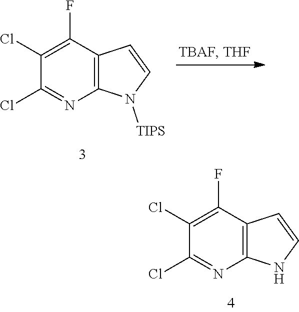

To a solution of (5,6-dichloro-4-fluoro-pyrrolo[2,3-b]pyridin-1-yl)-triisopropyl-silane (9.14 g, 25.30 mmol, 1 eq) in THF (100 mL) was added TBAF (1 M in THF, 30.37 mL, 1.2 eq). The mixture was stirred at 25° C. for 30 min. TLC indicated reactant 3 was consumed completely. The reaction mixture was concentrated under reduced pressure. The crude product was triturated with water (50 mL) at 25° C. for 30 min and filtered. The cake was collected and triturated with petroleum ether (50 mL) at 25° C. for 30 min and filtered. The cake was collected and triturated with CH₃CN (50 mL) at 25° C. for 30 min, and then filtered to afford 5,6-dichloro-4-fluoro-1H-pyrrolo[2,3-b]pyridine (4.23 g, 16.51 mmol, 65.23% yield, 80% purity) as a yellow solid. 1H NMR was recorded.

### Synthesis of 5,6-dichloro-4-fluoro-1-(p-tolylsulfonyl)pyrrolo[2,3-b]pyridine

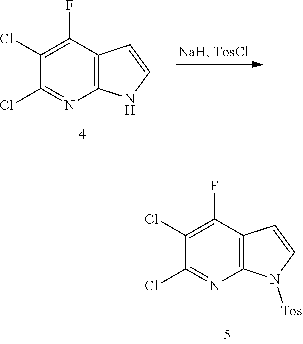

To a cooled solution of 5,6-dichloro-4-fluoro-1H-pyrrolo[2,3-b]pyridine (5.82 g, 28.39 mmol, 1 eq) in THE (70 mL) was added NaH (1.70 g, 42.58 mmol, 60% purity, 1.5 eq) in batches. After stirring at 0° C. for 30 min. TosCl (6.49 g, 34.07 mmol, 1.2 eq) was added in batches. The mixture was stirred at 0° C. for 30 min. TLC indicated reactant 4 was consumed completely. The reaction mixture was quenched with NH₄Cl solution (50 mL) at 25° C., diluted with water (20 mL), and then extracted with EtOAc (100 mL×2). The combined organic layer was washed with brine (50 mL×2), dried over Na₂SO₄, filtered and concentrated under reduced pressure to give a residue which was purified by column chromatography (SiO₂, petroleum ether/ethyl acetate=1/0 to 5/1) to afford 5,6-dichloro-4-fluoro-1-(p-tolylsulfonyl)pyrrolo[2,3-b]pyridine (6.44 g, 17.03 mmol, 50.00% yield, 95% purity) as a brown solid. ¹H NMR was recorded.

### Synthesis of methyl 5,6-dichloro-4-fluoro-1-(p-tolylsulfonyl)pyrrolo[2,3-b]pyridine-2-carboxylate

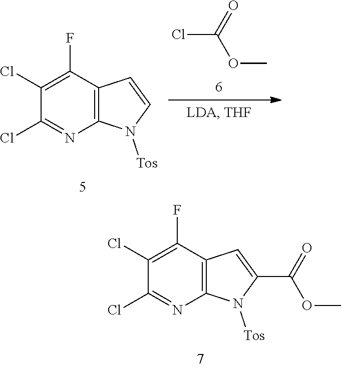

A mixture of 5,6-dichloro-4-fluoro-1-(p-tolylsulfonyl)pyrrolo[2,3-b]pyridine (2 g, 5.57 mmol, 1 eq) in THE (20 mL) was degassed and purged with N₂ for 3 times. LDA (2 M in THF, 4.18 mL, 1.5 eq) was then added. The reaction mixture was stirred at −60° C. for 10 min under N₂ atmosphere. To the mixture was then added methyl carbonochloridate (2.63 g, 27.84 mmol, 2.16 mL, 5 eq) and stirred at −60° C. for 30 min. TLC showed one major new spot. The reaction mixture was quenched by addition of saturated NH₄Cl solution (50 mL) at 25° C., and then diluted with water (50 mL) and extracted with EtOAc (50 mL×2). The combined organic layer was washed with brine (50 mL×2), dried over Na₂SO₄, filtered and concentrated under reduced pressure to give a residue which was purified by column chromatography (SiO₂, petroleum ether/ethyl acetate=1/0 to 5/1) to afford methyl 5,6-dichloro-4-fluoro-1-(p-tolylsulfonyl)pyrrolo[2,3-b]pyridine-2-carboxylate (1.46 g, 2.80 mmol, 40.17% yield, 80% purity) as a yellow solid. ¹H NMR was recorded.

### Synthesis of methyl 5-chloro-4-fluoro-6-methyl-1-(p-tolylsulfonyl)pyrrolo[2,3-b]pyridine-2-carboxylate

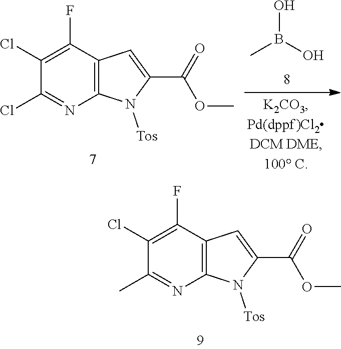

To a mixture of methyl 5,6-dichloro-4-fluoro-1-(p-tolylsulfonyl)pyrrolo[2,3-b]pyridine-2-carboxylate (1.16 g, 2.78 mmol, 1 eq), methylboronic acid (216.35 mg, 3.61 mmol, 1.3 eq) and K₂CO₃ (768.48 mg, 5.56 mmol, 2 eq) was added DME (5 mL). The mixture was purged with N₂ and Pd(dppf)Cl₂.CH₂Cl₂ (227.04 mg, 278.02 umol, 0.1 eq) was then added under N₂. The mixture was stirred at 100° C. for 12 hr. LC-MS showed desired mass. The mixture was filtered. The cake was washed with EtOAc (10 mL×2). The combined filtrate was dried over Na₂SO₄ and concentrated in vacuo. The resulting residue was purified by column chromatography (SiO₂, petroleum ether/ethyl acetate=1/0 to 10/1) to afford methyl 5-chloro-4-fluoro-6-methyl-1-(p-tolylsulfonyl)pyrrolo[2,3-b]pyridine-2-carboxylate (558 mg, 1.27 mmol, 45.52% yield, 90% purity) as a yellow solid. ¹H NMR was recorded.

### Synthesis of methyl 5-chloro-4-fluoro-6-methyl-1H-pyrrolo[2,3-b]pyridine-2-carboxylate

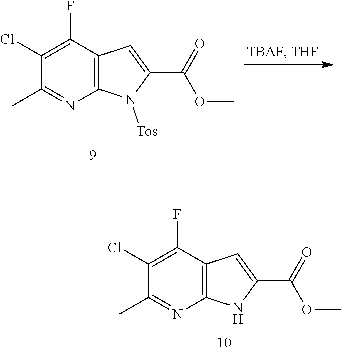

To a solution of methyl 5-chloro-4-fluoro-6-methyl-1-(p-tolylsulfonyl)pyrrolo[2,3-b]pyridine-2-carboxylate (612 mg, 1.54 mmol, 1 eq) in THE (5 mL) was added TBAF (1 M in THF, 2.00 mL, 1.3 eq). The mixture was stirred at 25° C. for 30 min. TLC indicated reactant 9 was consumed completely. The reaction mixture was concentrated under reduced pressure. The crude product was triturated with water (10 mL) at 25° C. for 30 min and filtered to afford methyl 5-chloro-4-fluoro-6-methyl-1H-pyrrolo[2,3-b]pyridine-2-carboxylate (370 mg, crude) as a yellow solid. 1H NMR was recorded.

### Synthesis of 5-chloro-4-fluoro-6-methyl-1H-pyrrolo[2,3-b]pyridine-2-carboxylic acid

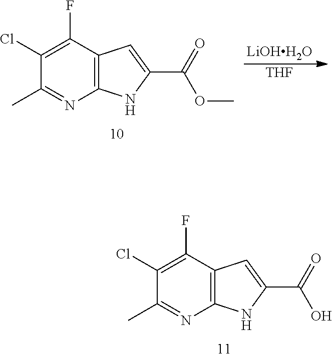

To a solution of methyl 5-chloro-4-fluoro-6-methyl-1H-pyrrolo[2,3-b]pyridine-2-carboxylate (370 mg, 1.52 mmol, 1 eq) in THF (3 mL) was added a solution of LiOH.H₂O (383.92 mg, 9.15 mmol, 6 eq) in H₂O (3 mL), and stirring at 30° C. for 12 hr. TLC indicated reactant 10 was consumed completely and one new spot formed. The reaction mixture was concentrated under reduced pressure to remove THF. To the aqueous phase was added aqueous HCl (6 M) until pH to 2, filtered and concentrated under reduced pressure to give 5-chloro-4-fluoro-6-methyl-1H-pyrrolo[2,3-b]pyridine-2-carboxylic acid (330 mg, 1.37 mmol, 89.93% yield, 95% purity, crude) as a yellow solid. The crude product was used for the next step without further purification.

¹H NMR (500 MHz, DMSO-d6) δ=12.60 (br s, 1H), 6.97 (d, J=1.5 Hz, 1H), 2.53-2.46 (m, 3H)

### Synthesis of 5-chloro-N-(1,1-dimethylsilinan-4-yl)-4-fluoro-6-methyl-1H-pyrrolo[2,3-b]pyridine-2-carboxamide

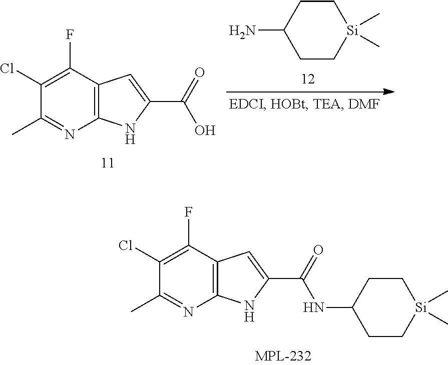

To a solution of 5-chloro-4-fluoro-6-methyl-1H-pyrrolo[2,3-b]pyridine-2-carboxylic acid (330 mg, 1.44 mmol, 1 eq) and 1,1-dimethylsilinan-4-amine (311.39 mg, 1.73 mmol, 1.2 eq, HCl) in DMF (3 mL) at 25° C. was added a solution of HOBt (585.16 mg, 4.33 mmol, 3 eq) and EDCI (830.18 mg, 4.33 mmol, 3 eq) in DMF (5 mL), followed by TEA (730.34 mg, 7.22 mmol, 1.00 mL, 5 eq). The mixture was stirred at 25° C. for 2 hr. LC-MS showed desired mass. The reaction mixture was quenched with aqueous NaHCO₃ (NaHCO₃:H₂O=2:1) (100 mL) at 25° C., filtered and concentrated under reduced pressure. The resulting residue was purified by column chromatography (SiO₂, petroleum ether/ethyl acetate=1/0 to 3/1) to afford 5-chloro-N-(1,1-dimethylsilinan-4-yl)-4-fluoro-6-methyl-1H-pyrrolo[2,3-b]pyridine-2-carboxamide (230 mg, 639.83 umol, 44.32% yield, 98.45% purity) as a yellow solid.

LCMS (ESI) m/z 354.0 [M+H]⁺; ¹H NMR (500 MHz, CHLOROFORM-d₃) δ=9.69 (br s, 1H), 7.33-7.21 (m, 1H), 6.78 (d, J=2.1 Hz, 1H), 6.06 (br d, J=7.9 Hz, 1H), 4.02-3.83 (m, 1H), 2.72 (s, 3H), 2.19 (td, J=3.7, 9.3 Hz, 2H), 1.60-1.52 (m, 2H), 0.86-0.65 (m, 4H), 0.08 (d, J=18.8 Hz, 6H).

### Example 136. MPL-274

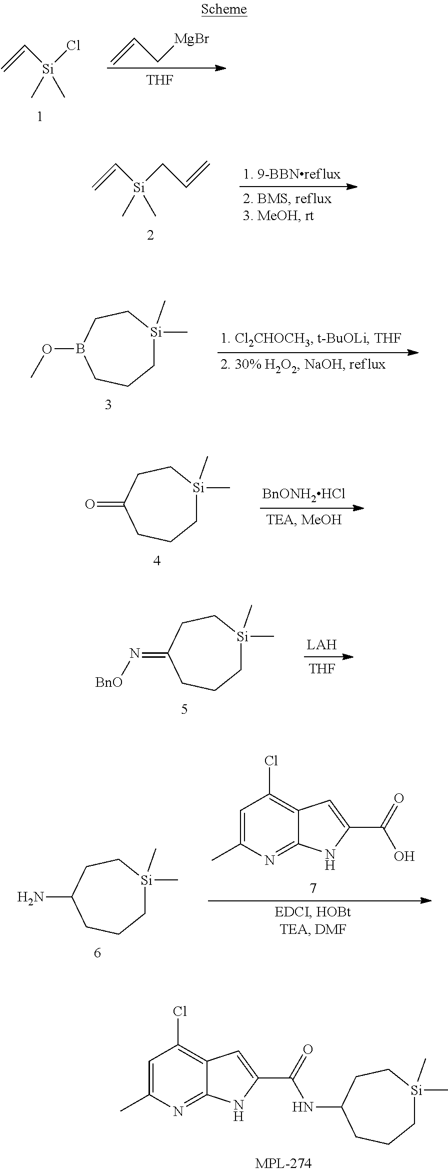

### Synthesis of allyl-dimethyl-vinyl-silane

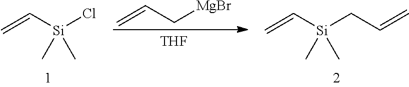

Chloro-dimethyl-vinyl-silane (30 g, 248.65 mmol, 1 eq) was added to allyl(bromo)magnesium (1 M, 497.30 mL, 2 eq) (in THF) at 30° C. under N₂. The mixture was stirred at 85° C. for 12 hr. TLC (petroleum ether) showed a new spot. The mixture was cooled to 0° C., then poured to saturated NH₄Cl (600 mL), and extracted with n-pentane (200 mL). The organic layer was dried with Na₂SO₄ and filtered. The solvent was removed by distillation under 15 Psi at 110° C. The product was distilled under reduced pressure at 30° C. Compound allyl-dimethyl-vinyl-silane (24 g, 142.55 mmol, 57.33% yield, 75% purity) was obtained as a colorless oil. ¹H NMR was recorded.

### Synthesis of 4-methoxy-1,1-dimethyl-1,4-silaborepane

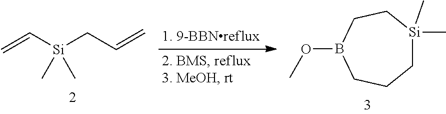

To a 1 L three necks flask purged with N₂ was added 9-BBN (0.5 M, 313.61 mL, 2.2 eq), followed by allyl-dimethyl-vinyl-silane (12 g, 71.28 mmol, 1 eq). The mixture was stirred at 80° C. under N2 for 3 hr. The mixture was cooled to 25° C. then BH₃-Me₂S (10 M, 8.55 mL, 1.2 eq) was added dropwise. The mixture was stirred at 80° C. for 2 hr. After cooled to 25° C., MeOH (18.27 g, 570.20 mmol, 23.07 mL, 8 eq) was added dropwise, the mixture was stirred at 25° C. for additional 12 hr. TLC (petroleum ether:EtOAc=10:1) showed two major spots. The solvents were removed by distillation at 120° C. (oil bath) under 15 Psi. Compound 4-methoxy-1,1-dimethyl-1,4-silaborepane (38 g, crude) was obtained as a light yellow oil.

### Synthesis of 1,1-dimethylsilepan-4-one

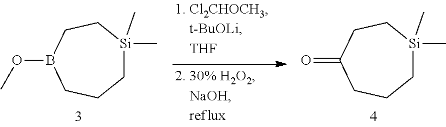

To a solution of 4-methoxy-1,1-dimethyl-1,4-silaborepane (38 g, 223.36 mmol, 1 eq) in t-BuOLi (2.2 M, 507.63 mL, 5 eq) was added dichloro(methoxy)methane (25.68 g, 223.36 mmol, 19.75 mL, 1 eq) dropwise at 0° C. with an ice-water bath. The mixture was stirred at 25° C. for 30 min. Then NaOH (26.80 g, 670.07 mmol, 3 eq) in a mixture of H₂O (32 mL) and EtOH (100 mL) was added, followed by dropwise addition of H₂O₂ (94.71 g, 835.35 mmol, 80.27 mL, 30% in H₂O, 3.74 eq). The solution was stirred at 90° C. for 3 hr. TLC (petroleum ether:EtOAc=10:1) showed one major spot. The reaction was quenched by water (500 mL), then extracted with ethyl acetate (2×100 ml). The aqueous layer was quenched with sat. Na₂SO₃ (100 mL). The organic layers were combined and dried over Na₂SO₄, filtered and distilled at 120° C. (oil bath) under 15 Psi to remove the solvent. Compound 1,1-dimethylsilepan-4-one (44 g, crude) was obtained as a yellow oil. ¹H NMR was recorded.

### Synthesis of (E)-N-benzyloxy-1,1-dimethyl-silepan-4-imine

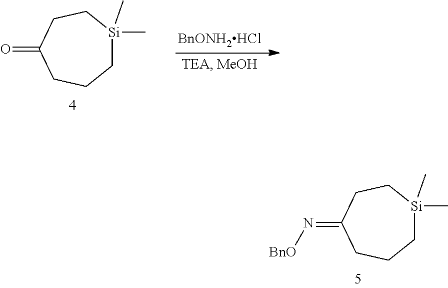

To an ice-cooled solution of 1,1-dimethylsilepan-4-one (43 g, 275.12 mmol, 1 eq) and TEA (55.68 g, 550.23 mmol, 76.59 mL, 2 eq) in MeOH (450 mL) was added O-benzylhydroxylamine (57.09 g, 357.65 mmol, 1.3 eq, HCl) at 0° C. The mixture was stirred at 25° C. for 12 hr. TLC (petroleum ether:EtOAc=50:1) showed one major spot under UV 254 nm and no spot stained by DNP (dinitrophenylhydrazine). The mixture was concentrated under reduced pressure to give a residue which was diluted with EtOAc (300 mL) and washed with water (200 mL) and brine (200 mL). The organic layer was dried with Na₂SO₄, filtered and concentrated to give a residue which was purified by flash silica gel chromatography (ISCO®; 220 g SepaFlash® Silica Flash Column; eluent of 0-3% ethyl acetate in petroleum ether at 100 mL/min). The fractions containing the product (checked by TLC; petroleum ether:EtOAc=50:1) were collected and concentrated to give light yellow oil (18 g), which was further purified in two batches (8 g and 10 g) by prep-HPLC (column: Boston Uni C18 40*150*5 um; mobile phase: A: 0.225% formic acid in water, B: CH₃CN; gradient: 70%-100%, B over 11 min) to afford (E)-N-benzyloxy-1,1-dimethyl-silepan-4-imine 5.0 g, 17.21 mmol, 6.25% yield, 90% purity) as a yellow oil. ¹H NMR was recorded.

### Synthesis of 1,1-dimethylsilepan-4-amine

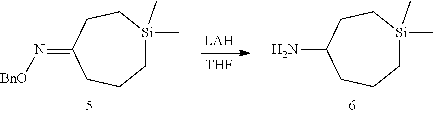

To an ice-cooled solution of (E)-N-benzyloxy-1,1-dimethyl-silepan-4-imine (0.5 g, 1.91 mmol, 1 eq) in THE (5 mL) was added LAH (145.16 mg, 3.83 mmol, 2 eq). The mixture was warmed to 25° C. and stirred for 1 hr, and then heated to 80° C. to reflux and stirred for 1.5 hr. TLC (petroleum ether:EtOAc=1:1) showed starting material was consumed completely, and several new spots formed. The reaction was quenched with 0.15 mL of water and 0.15 mL of aq. NaOH (15% in water), followed with 0.45 mL of water and Na₂SO₄, and then filtered. The cake was washed with MeOH/DCM (1:10, 5 mL×3). The pH of combined filtrate was adjusted to 2 with HCl in MeOH, and the mixture was stirred at 25° C. for 2 hr and then concentrated under reduced pressure. The resulting residue was diluted with EtOAc (25 mL), and then extracted with water (7 mL×4). The aqueous layers were combined, and then dried by lyophilizer to give 1,1-dimethylsilepan-4-amine (310 mg, 1.44 mmol, 75.28% yield, 90% purity, HCl salt) as a white solid.

¹H NMR (500 MHz, DMSO-d₆) δ=7.91 (br s, 3H), 3.02 (br s, 1H), 2.03-1.91 (m, 2H), 1.87-1.77 (m, 1H), 1.64-1.52 (m, 1H), 1.46-1.36 (m, 2H), 0.81-0.68 (m, 2H), 0.63-0.53 (m, 2H), 0.01 (d, J=9.8 Hz, 6H).

### Synthesis of 4-chloro-N-(1,1-dimethylsilepan-4-yl)-6-methyl-1H-pyrrolo[2,3-b]pyridine-2-carboxamide

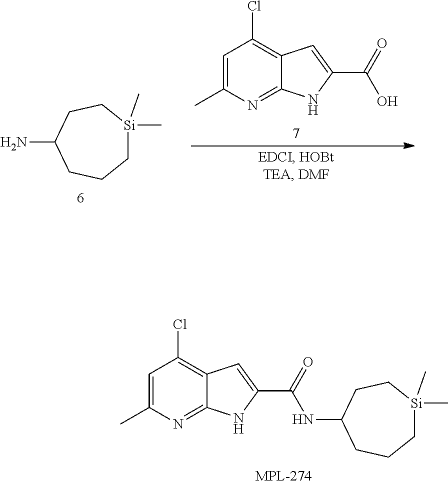

To a solution of 4-chloro-6-methyl-1H-pyrrolo[2,3-b]pyridine-2-carboxylic acid (550 mg, 2.61 mmol, 1 eq) and 1,1-dimethylsilepan-4-amine (657.87 mg, 3.39 mmol, 1.3 eq, HCl) in DMF (7 mL) was added a solution of EDCI (1.00 g, 5.22 mmol, 2 eq) and HOBt (705.71 mg, 5.22 mmol, 2 eq) in DMF (7 mL), followed by TEA (1.06 g, 10.45 mmol, 1.45 mL, 4 eq). The mixture was stirred at 25° C. for 2 hrs. LC-MS showed the acid was consumed completely and one main peak with desired mass was detected. The mixture was poured into water, the crude product was isolated as red solid and collected by filtration. The residue was purified by flash silica gel chromatography (ISCO®; 12 g SepaFlash® Silica Flash Column, 0-15% ethyl acetate in petroleum ether at 40 mL/min). All fractions containing the desired product (checked by TLC (petroleum ether:EtOAc=3:1) were combined and concentrated. The residue was dried by lyophilizer. Compound 4-chloro-N-(1,1-dimethylsilepan-4-yl)-6-methyl-1H-pyrrolo[2,3-b]pyridine-2-carboxamide (490 mg, 1.34 mmol, 51.44% yield, 95.929% purity) was obtained as a light yellow solid.

LCMS (ESI) m/z 350.1 [M+H]⁺; ¹H NMR (500 MHz, DMSO-d₆) 3=12.21-12.17 (m, 1H), 12.19 (br s, 1H), 8.33-8.21 (m, 1H), 8.30 (br d, J=8.1 Hz, 1H), 7.15 (s, 1H), 7.11 (s, 1H), 3.90-3.79 (m, 1H), 2.48 (s, 3H), 1.91-1.73 (m, 3H), 1.69-1.59 (m, 1H), 1.51-1.39 (m, 2H), 0.76-0.65 (m, 2H), 0.61-0.53 (m, 2H), −0.01 (d, J=9.3 Hz, 6H).

### Example 137. MPL-275

### Synthesis of 4-chloro-N-(1,1-dimethylsilocan-4-yl)-6-methyl-1H-pyrrolo[2,3-b]pyridine-2-carboxamide

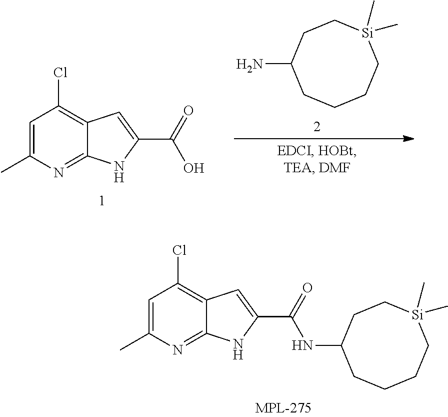

To a solution of 4-chloro-6-methyl-1H-pyrrolo[2,3-b]pyridine-2-carboxylic acid (25 mg, 118.70 umol, 1.2 eq) and 1,1-dimethylsilocan-4-amine (20.56 mg, 98.92 umol, 1 eq, HCl) in DMF (0.5 mL) was added a solution of EDCI (37.92 mg, 197.83 umol, 2 eq) and HOBt (26.73 mg, 197.83 umol, 2 eq) in DMF (0.5 mL), followed by TEA (40.04 mg, 395.66 umol, 55.07 uL, 4 eq). The mixture was stirred at 20° C. for 2 hr. LC-MS showed reactant 1 was consumed completely and one main peak with desired mass was detected. The mixture was diluted with MeOH (2 mL) and filtered to remove insoluble matter. The residue was purified by prep-HPLC (column: YMC-Actus Triart C18 150*30 mm*5 um; mobile phase: A: 0.225% formic acid in water, B: CH3CN; gradient: 70%-100% B % over 11 min). Compound 4-chloro-N-(1,1-dimethylsilocan-4-yl)-6-methyl-1H-pyrrolo[2,3-b]pyridine-2-carboxamide (20 mg, 54.95 umol, 55.55% yield, 100% purity) was obtained as a white solid.

LCMS (ESI) m/z 364.1 [M+H]⁺; ¹H NMR (500 MHz, DMSO-d₆) δ=12.24 (br s, 1H), 8.30 (d, J=7.9 Hz, 1H), 7.27-7.19 (m, 1H), 7.15 (s, 1H), 4.08-3.89 (m, 1H), 2.51 (s, 3H), 1.81-1.52 (m, 7H), 1.46-1.35 (m, 1H), 0.84-0.75 (m, 1H), 0.72-0.61 (m, 2H), 0.55 (ddd, J=3.0, 8.5, 15.2 Hz, 1H), 0.59-0.51 (m, 1H), 0.01 (d, J=11.9 Hz, 6H).

### Example 138. MPL-276

### Synthesis of 4-chloro-6-methyl-N-(5-silaspiro[4.5]decan-8-yl)-1H-pyrrolo[2,3-b]pyridine-2-carboxamide

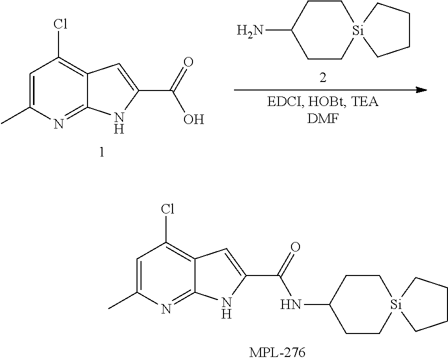 [SMILES: Cc1cc(Cl)c2cc(C(=O)O)[nH]c2n1 ; NC1CC[Si]2(CCCC2)CC1 ; Cc1cc(Cl)c2cc(C(=O)NC3CC[Si]4(CCCC4)CC3)[nH]c2n1]

To a solution of 4-chloro-6-methyl-1H-pyrrolo[2,3-b]pyridine-2-carboxylic acid (50 mg, 237.40 umol, 1 eq) in DMF (1 mL) was added 5-silaspiro[4.5]decan-8-amine (53.74 mg, 261.14 umol, 1.1 eq. HCl salt). Then a solution of HOBt (64.16 mg, 474.80 umol, 2 eq) and EDCI (91.02 mg, 474.80 umol, 2 eq) in DMF (1 mL) was added to the mixture, followed by TEA (96.09 mg, 949.59 umol, 132.17 uL, 4 eq). The mixture was stirred at 25° C. for 2 hrs. LCMS showed one main peak with desired mass. DMF (3 mL) was added to the mixture, filtered to collect the filtrate which was purified by prep-HPLC (column: YMC-Actus Triart C18 100*30 mm*5 um; mobile phase: A: 0.225% formic acid in water, B: CH₃CN; gradient: 65%-85% B over 11 min). Compound 4-chloro-6-methyl-N-(5-silaspiro[4.5]decan-8-yl)-1H-pyrrolo[2,3-b]pyridine-2-carboxamide (61.5 mg, 169.92 umol, 71.57% yield, 100% purity) was obtained as a white solid.

LCMS (ESI) m/z 362.1 [M+H]⁺; ¹H NMR (500 MHz, DMSO-d₆): 3=12.26 (s, 1H) 8.33 (d, J=8.09 Hz, 1H) 7.20 (s, 1H) 7.17 (s, 1H) 3.77 (td, J=11.02, 8.01 Hz, 1H) 2.54 (s, 4H) 2.04-2.12 (m, 2H) 1.54-1.65 (m, 6H) 0.72-0.87 (m, 4H) 0.63 (br t, J=6.71 Hz, 2H) 0.55 (br t, J=6.79 Hz, 2H).

### Example 139. MPL-277

### Synthesis of 4-chloro-6-methyl-N-(6-silaspiro[5.5]undecan-3-yl)-1H-pyrrolo[2,3-b]pyridine-2-carboxamide

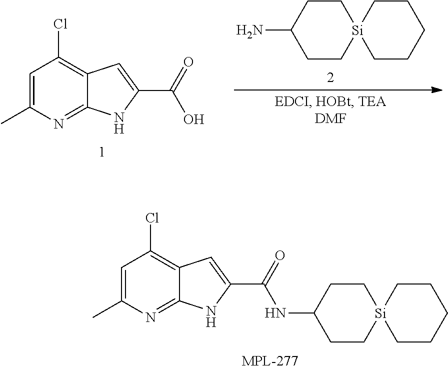 [SMILES: Cc1cc(Cl)c2cc(C(=O)O)[nH]c2n1 ; NC1CC[Si]2(CCCCC2)CC1 ; Cc1cc(Cl)c2cc(C(=O)NC3CC[Si]4(CCCCC4)CC3)[nH]c2n1]

To a solution of 4-chloro-6-methyl-1H-pyrrolo[2,3-b]pyridine-2-carboxylic acid (50 mg, 237.40 umol, 1 eq) in DMF (1 mL) was added 6-silaspiro[5.5]undecan-3-amine (57.41 mg, 261.14 umol, 1.1 eq, HCl salt). Then a solution of HOBt (64.16 mg, 474.80 umol, 2 eq) and EDCI (91.02 mg, 474.80 umol, 2 eq) in DMF (1 mL) was added, and followed by TEA (96.09 mg, 949.60 umol, 132.17 uL, 4 eq). The mixture was stirred at 25° C. for 2 hr. LCMS showed one main peak with desired mass. DMF (3 mL) was added. The mixture was filtered. The filtrate was purified by prep-HPLC (column: YMC-Actus Triart C18 100*30 mm*5 um; mobile phase: A: 0.225% formic acid in water, B: CH₃CN; 75%-100% B over 11 min) to give desired compound 4-chloro-6-methyl-N-(6-silaspiro[5.5]undecan-3-yl)-1H-pyrrolo[2,3-b]pyridine-2-carboxamide (68 mg, 180.87 umol, 76.19% yield, 100% purity) as a white solid.

LCMS (ESI) m/z 376.1 [M+H]⁺; ¹H NMR (500 MHz, DMSO-d₆) δ=12.26 (s, 1H) 8.33 (br d, J=7.93 Hz, 1H) 7.19 (s, 1H) 7.17 (s, 1H) 3.67-3.80 (m, 1H) 2.54 (s, 3H) 2.01 (br d, J=9.92 Hz, 2H) 1.53-1.74 (m, 6H) 1.40 (br s, 2H) 0.91 (br d, J=14.50 Hz, 2H) 0.67-0.77 (m, 2H) 0.56-0.66 (m, 4H).

### Example 140. MPL-280

### Synthesis of 4-fluoro-3,6-dimethyl-N-(5-silaspiro[4.5]decan-8-yl)-1H-pyrrolo[2,3-b]pyridine-2-carboxamide

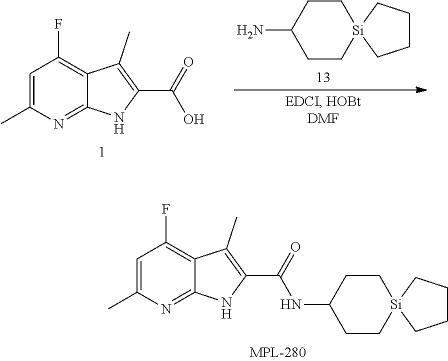 [SMILES: Cc1cc(F)c2c(C)c(C(=O)O)[nH]c2n1 ; NC1CC[Si]2(CCCC2)CC1 ; Cc1cc(F)c2c(C)c(C(=O)NC3CC[Si]4(CCCC4)CC3)[nH]c2n1]

To a solution of 4-fluoro-3,6-dimethyl-1H-pyrrolo[2,3-b]pyridine-2-carboxylic acid (30 mg, 144.10 umol, 1 eq) and 5-silaspiro[4.5]decan-8-amine (35.59 mg, 172.92 umol, 1.2 eq, HCl salt) in DMF (1 mL) was added a solution of EDCI (55.25 mg, 288.20 umol, 2 eq) and HOBt (38.94 mg, 288.20 umol, 2 eq) in DMF (1 mL), followed by TEA (58.33 mg, 576.40 umol, 80.23 uL, 4 eq). The mixture was stirred at 25° C. for 12 hr. LC-MS showed one main peak with desired mass. The mixture was filtered to remove insoluble matter. The filtrate was purified by prep-HPLC (column: YMC-Actus Triart C18 150*30 mm*5 um; mobile phase: A: 0.225% formic acid in water, B: CH₃CN; gradient: 65%-95% B over 11 min). Compound 4-fluoro-3,6-dimethyl-N-(5-silaspiro[4.5]decan-8-yl)-1H-pyrrolo[2,3-b]pyridine-2-carboxamide (35 mg, 96.83 umol, 67.20% yield, 99.46% purity) was obtained as a white solid.

LCMS (ESI) m/z 360.1 [M+H]⁺; ¹H NMR (500 MHz, DMSO-d₆) δ=11.86 (br s, 1H), 7.80 (br d, J=7.6 Hz, 1H), 6.83 (d, J=12.1 Hz, 1H), 3.82-3.71 (m, 1H), 2.57 (s, 3H), 2.53-2.52 (m, 3H), 2.09 (br d, J=10.4 Hz, 2H), 1.66-1.54 (m, 6H), 0.87-0.79 (m, 2H), 0.78-0.69 (m, 2H), 0.64-0.52 (m, 4H).

### Example 141. MPL-281

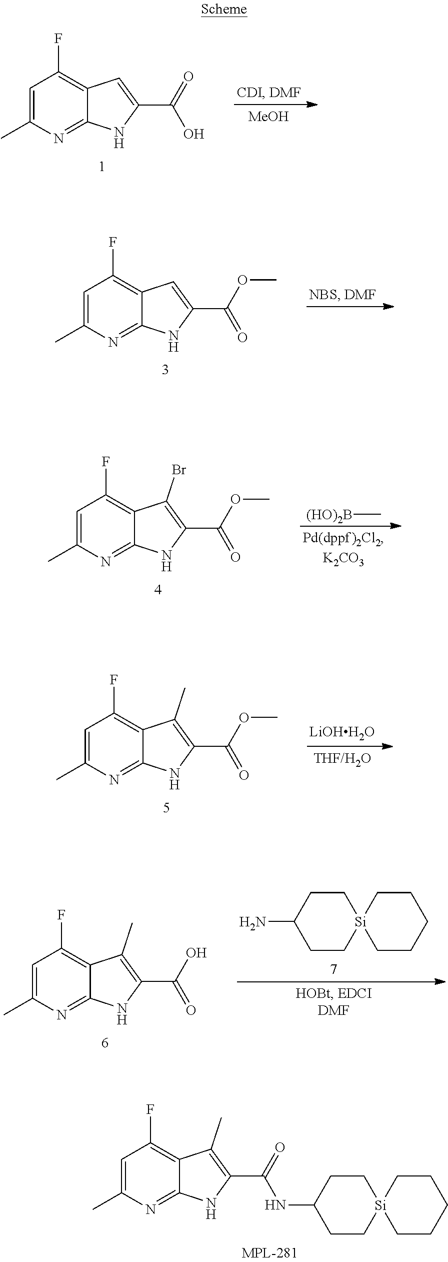

### Synthesis of 4-fluoro-6-methyl-1H-pyrrolo[2,3-b]pyridine-2-carboxylate

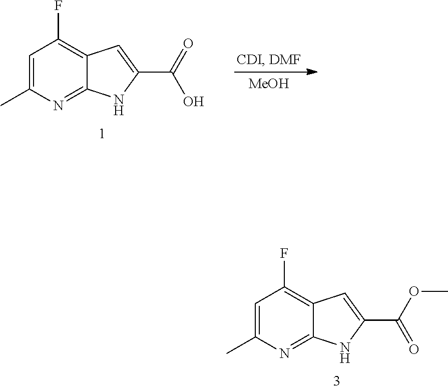

A solution of 4-fluoro-6-methyl-1H-pyrrolo[2,3-b]pyridine-2-carboxylic acid (3 g, 15.45 mmol, 1 eq) and CDI (2.76 g, 17.00 mmol, 1.1 eq) in DMF (30 mL) was stirred at 30° C. for 1 hr. MeOH (23.75 g, 741.35 mmol, 30.00 mL, 47.98 eq) was then added and the reaction was stirred for 30 min. LCMS showed desired mass. The mixture was concentrated under reduced pressure to remove CH₃OH, and then poured into water (200 mL). The resulting suspension was filtered. The filter cake was collected, diluted with EtOAc (100 mL), dried over Na2SO4, filtered and concentrated under reduced pressure to give methyl 4-fluoro-6-methyl-1H-pyrrolo[2,3-b]pyridine-2-carboxylate (1.6 g, 7.30 mmol, 40.74% yield, 95% purity) as a yellow solid.

LCMS (ESI) m/z 209.2[M+H]⁺; ¹H NMR was recorded.

### Synthesis of 3-bromo-4-fluoro-6-methyl-1H-pyrrolo[2,3-b]pyridine-2-carboxylate

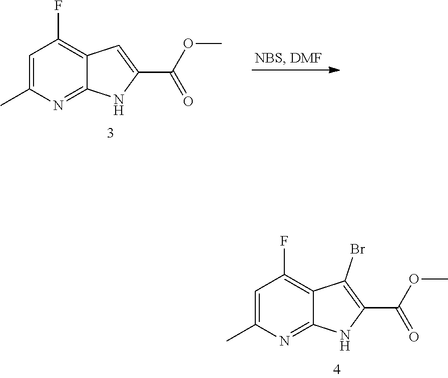

A solution of methyl 4-fluoro-6-methyl-1H-pyrrolo[2,3-b]pyridine-2-carboxylate (1.5 g, 7.21 mmol, 1 eq) and NBS (1.41 g, 7.93 mmol, 1.1 eq) in DMF (30 mL) under N₂ was stirred at 30° C. for 3 hr. LCMS showed desired mass. The mixture was poured into water (200 mL). The suspension was filtered. The filter cake was washed with water (20 mL) and dried in vacuo to afford methyl 3-bromo-4-fluoro-6-methyl-1H-pyrrolo[2,3-b]pyridine-2-carboxylate (1.2 g, 3.97 mmol, 55.11% yield, 95% purity) as a yellow solid.

LCMS (ESI) m/z 289.0 [M+H]⁺; ¹H NMR was recorded.

### Synthesis of methyl 4-fluoro-3,6-dimethyl-H-pyrrolo[2,3-b]pyridine-2-carboxylate

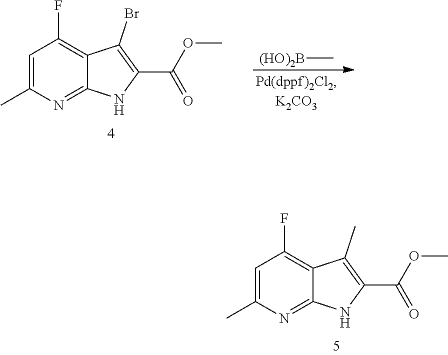

To a mixture of methyl 3-bromo-4-fluoro-6-methyl-1H-pyrrolo[2,3-b]pyridine-2-carboxylate (600 mg, 2.09 mmol, 1 eq), methylboronic acid (625.54 mg, 10.45 mmol, 5 eq) and Cs₂CO₃ (2.04 g, 6.27 mmol, 3 eq) in dioxane (10 mL) and H₂O (0.1 mL) was added Pd(dppf)Cl₂.CH₂Cl₂ (170.68 mg, 209.00 umol, 0.1 eq) under N₂. The reaction mixture was stirred at 110° C. for 12 hr. LCMS showed desire mass. The mixture was filtered. The filtrate was concentrated under reduced pressure. The resulting residue was purified by column chromatography (SiO₂, petroleum ether/ethyl aceate=50/1 to 1/1) to afford methyl 4-fluoro-3,6-dimethyl-1H-pyrrolo[2,3-b]pyridine-2-carboxylate (110 mg, 346.51 umol, 16.58% yield, 70% purity) as a white solid.

LCMS (ESI) m/z 223.1 [M+H]⁺; ¹H NMR was recorded.

### Synthesis of 4-fluoro-3,6-dimethyl-1H-pyrrolo[2,3-b]pyridine-2-carboxylic acid

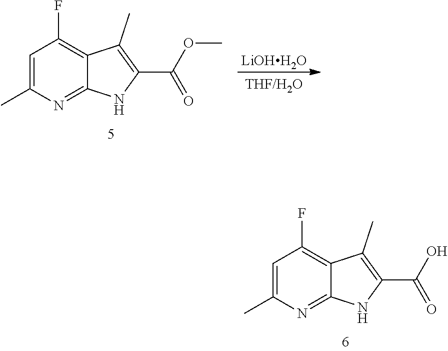

To a solution of methyl 4-fluoro-3,6-dimethyl-1H-pyrrolo[2,3-b]pyridine-2-carboxylate (362 mg, 1.63 mmol, 1 eq) in THE (5 mL) was added a solution of LiOH.H₂O (410.17 mg, 9.77 mmol, 6 eq) in H₂O (5 mL). The mixture was stirred at 30° C. for 2 hr. LCMS showed desired mass. The mixture was concentrated under reduced pressure to remove THF. The aqueous solution was adjusted to pH to 4 with aq. HCl (6M). The suspension was filtered, and the filter cake was washed with water (10 mL) and collected. Compound 4-fluoro-3,6-dimethyl-1H-pyrrolo[2,3-b]pyridine-2-carboxylic acid (339 mg, crude) was obtained as a white solid.

LCMS (ESI) m/z 209.1[M+H]⁺; ¹H NMR was recorded.

### Synthesis of 4-fluoro-3,6-dimethyl-N-(6-silaspiro[5.5]undecan-3-yl)-1H-pyrrolo[2,3-b]pyridine-2-carboxamide

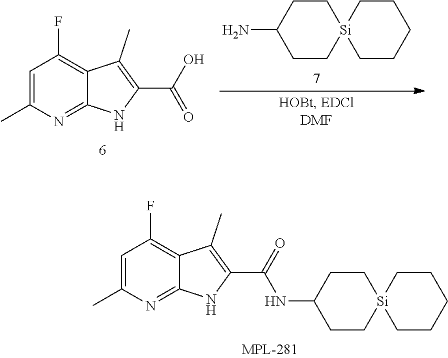 [SMILES: Cc1cc(F)c2c(C)c(C(=O)O)[nH]c2n1 ; NC1CC[Si]2(CCCCC2)CC1 ; Cc1cc(F)c2c(C)c(C(=O)NC3CC[Si]4(CCCCC4)CC3)[nH]c2n1]

To a solution of 4-fluoro-3,6-dimethyl-1H-pyrrolo[2,3-b]pyridine-2-carboxylic acid (289 mg, 1.39 mmol, 1 eq) and 6-silaspiro[5.5]undecan-3-amine (335.67 mg, 1.53 mmol, 1.1 eq, HCl) in DMF (3 mL) was added a solution of EDCI (798.34 mg, 4.16 mmol, 3 eq) and HOBt (562.72 mg, 4.16 mmol, 3 eq) in DMF (2 mL), followed by TEA (702.33 mg, 6.94 mmol, 966.07 uL, 5 eq). The mixture was stirred at 20° C. for 1 hr. LCMS showed desired mass. The mixture was filtered, and the filtrate was purified by prep-HPLC (column: YMC-Actus Triart C18 150*30 mm*5 um; mobile phase: A: 0.225% formic acid in water, B: CH₃CN; gradient: 75%-100% B over 13 min) to afford 4-fluoro-3,6-dimethyl-N-(6-silaspiro[5.5]undecan-3-yl)-1H-pyrrolo[2,3-b]pyridine-2-carboxamide (166.5 mg, 427.33 umol, 30.78% yield, 95.87% purity) as a white solid.

LCMS (ESI) m/z 274.1[M+H]⁺; ¹H NMR (500 MHz, DMSO-d6)=0.51-0.65 (m, 4H) 0.65-0.73 (m, 2H) 0.82-0.95 (m, 2H) 1.39 (br s, 2H) 1.51-1.73 (m, 6H) 1.94-2.09 (m, 2H) 2.52 (br s, 3H) 2.54-2.60 (m, 3H) 3.65-3.82 (m, 1H) 6.82 (d, J=12.05 Hz, 1H) 7.79 (br d, J=7.63 Hz, 1H) 11.88 (br s, 1H).

### Example 142. MPL-282, MPL-282A and MPL-282B

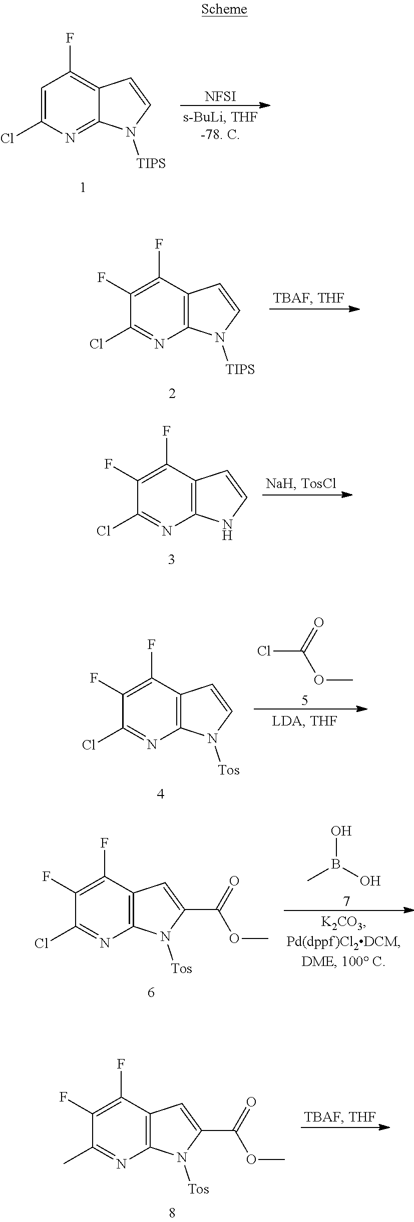

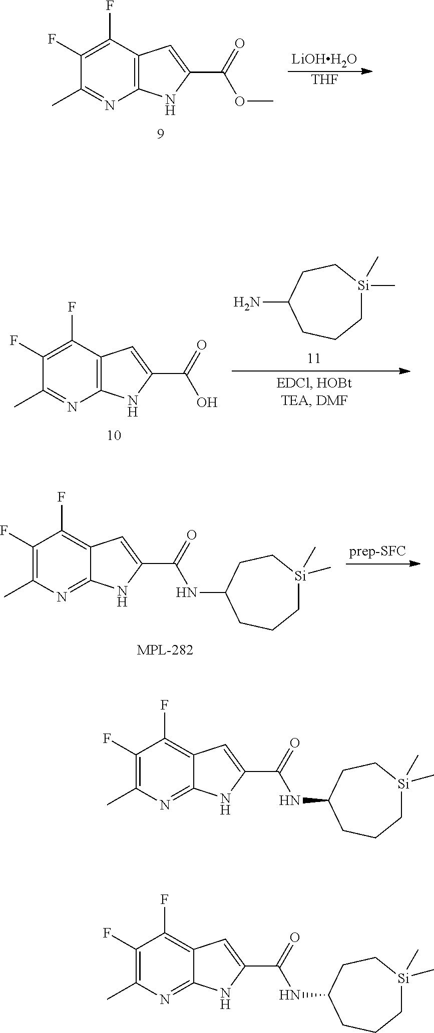

### Synthesis of (6-chloro-4,5-difluoro-pyrrolo[2,3-b]pyridin-1-yl)-triisopropyl-silane

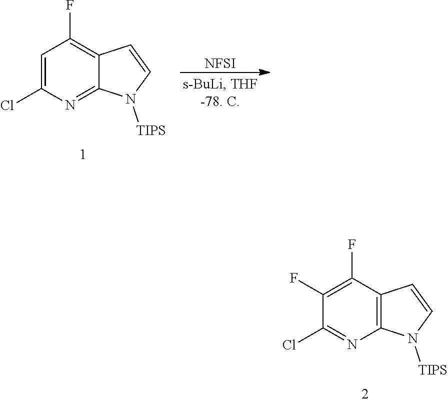

To a solution of (6-chloro-4-fluoro-pyrrolo[2,3-b]pyridin-1-yl)-triisopropyl-silane (10 g, 30.59 mmol, 1 eq) in THE (100 mL) was added a solution of s-BuLi (1.3 M in n-hexane, 44.71 mL, 1.9 eq). The mixture was stirred at −78° C. for 0.5 hr. Then a solution of NFSI (28.94 g, 91.77 mmol, 3 eq) in THE (100 mL) was added. The reaction mixture was stirred at −78° C. for 1.5 hr. LC-MS showed desired mass. The reaction mixture was quenched by addition of saturated NH₄Cl (100 mL) at −78° C., and then extracted with EtOAc (200 mL×3). The combined organic layer was dried over Na₂SO₄, filtered and concentrated under reduced pressure. The resulting residue was purified by column chromatography (SiO₂, petroleum ether/ethyl acetate=1/0 to 5/1) to afford (6-chloro-4,5-difluoro-pyrrolo[2,3-b]pyridin-1-yl)-triisopropyl-silane (9 g, 23.48 mmol, 76.77% yield, 90% purity) as a yellow oil.

LCMS (ESI) m/z 345.2 [M+H]⁺; ¹H NMR was recorded.

### Synthesis of 6-chloro-4,5-difluoro-1H-pyrrolo[2,3-b]pyridine

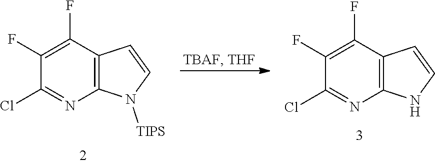

To a solution of (6-chloro-4,5-difluoro-pyrrolo[2,3-b]pyridin-1-yl)-triisopropyl-silane (9 g, 26.09 mmol, 1 eq) in THE (50 mL) was added TBAF (1 M in THF, 39.14 mL, 1.5 eq). The mixture was stirred at 25° C. for 30 min. TLC indicated the reaction was completed. The reaction mixture was concentrated under reduced pressure. The resulting residue was triturated with water (100 mL) for 20 min, filtered, and the filter cake was washed with petroleum ether (20 mL×3). The cake was dried under reduced pressure. Compound 6-chloro-4,5-difluoro-1H-pyrrolo[2,3-b]pyridine (3.2 g, 15.27 mmol, 58.53% yield, 90% purity) was obtained as a yellow solid. ¹H NMR was recorded. The crude product was used for the next step without purification.

### Synthesis of 6-chloro-4,5-difluoro-1-(p-tolylsulfonyl)pyrrolo[2,3-b]pyridine

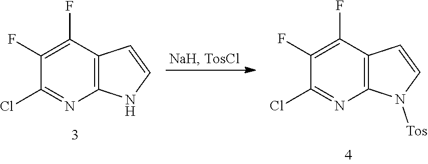

To a solution of 6-chloro-4,5-difluoro-1H-pyrrolo[2,3-b]pyridine (5.3 g, 28.11 mmol, 1 eq) in THE (50 mL) was added NaH (3.37 g, 84.32 mmol, 60% purity, 3 eq) at 0° C. under N₂, followed by the solution of TosCl (8.04 g, 42.16 mmol, 1.5 eq) in THE (30 mL) dropwise at 0° C. The reaction mixture was then stirred at 0° C. for 0.5 hr. LC-MS showed desired compound was detected. The reaction mixture was poured into saturated NH₄Cl (100 mL), and then extracted with EtOAc (100 mL×3). The organic layers were combined, dried over Na₂SO₄, filtered and concentrated under reduced pressure. The resulting residue was purified by column chromatography (SiO₂, petroleum ether/tthyl acetate=1/0 to 10/1) to afford 6-chloro-4,5-difluoro-1-(p-tolylsulfonyl)pyrrolo[2,3-b]pyridine (7.7 g, 22.47 mmol, 79.93% yield) as a yellow solid.

LCMS (ESI) m/z 343.0 [M+H]⁺; ¹H NMR was recorded.

### Synthesis of methyl 6-chloro-4,5-difluoro-1-(p-tolylsulfonyl)pyrrolo[2,3-b]pyridine-2-carboxylate

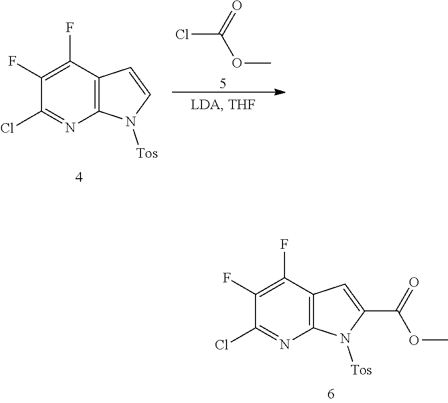

A mixture of 6-chloro-4,5-difluoro-1-(p-tolylsulfonyl)pyrrolo[2,3-b]pyridine (6.7 g, 19.55 mmol, 1 eq) in THE (70 mL) was degassed and purged with N₂ for 3 times. LDA (2 M in THF, 14.66 mL, 1.5 eq) and stirred at −78° C. for 10 min under N₂ atmosphere. Methyl carbonochloridate (9.24 g, 97.74 mmol, 7.57 mL, 5 eq) was then added. The mixture was stirred at −78° C. for 30 min. LC-MS showed desire mass. The reaction mixture was quenched with saturated NH₄Cl solution (50 mL) at 25° C., and then diluted with water (50 mL), and extracted with EtOAc (100 mL×2). The combined organic layer was washed with brine (100 mL×2), dried over Na₂SO₄, filtered and concentrated under reduced pressure. The resulting residue was purified by column chromatography (SiO₂, petroleum ether/ethyl acetate=1/0 to 10/1) to afford methyl 6-chloro-4,5-difluoro-1-(p-tolylsulfonyl)pyrrolo[2,3-b]pyridine-2-carboxylate (3.1 g, 6.19 mmol, 31.65% yield, 80% purity) as a yellow solid.

LCMS (ESI) m/z 401.1 [M+H]⁺; ¹H NMR was recorded.

### Synthesis of methyl 4,5-difluoro-6-methyl-1-(p-tolylsulfonyl)pyrrolo[2,3-b]pyridine-2-carboxylate

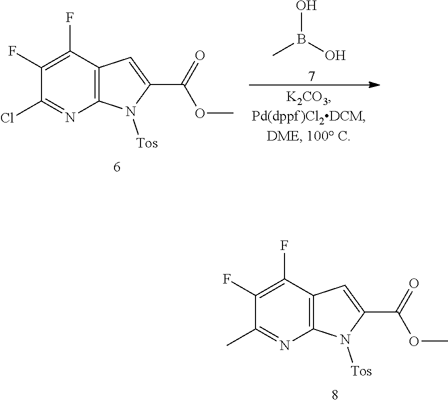

To a mixture of methyl 6-chloro-4,5-difluoro-1-(p-tolylsulfonyl)pyrrolo[2,3-b]pyridine-2-carboxylate (3.1 g, 7.73 mmol, 1 eq) in DME (30 mL) was added methylboronic acid (2.32 g, 38.67 mmol, 5 eq) and K₂CO₃ (3.21 g, 23.20 mmol, 3 eq). The mixture was purged with N₂ and Pd(dppf)Cl₂.CH₂Cl₂ (631.66 mg, 773.48 umol, 0.1 eq) was added under N₂. The mixture was stirred at 100° C. for 12 hr. LC-MS showed desired mass. The reaction mixture was filtered, the cake was washed with EtOAc (10 mL×3). The combined organic layer was washed with brine (50 mL×2), dried over Na₂SO₄, filtered and concentrated under reduced pressure. The resulting residue was purified by column chromatography (SiO₂, petroleum ether/ethyl acetate=1/0 to 10/1) to afford methyl 4,5-difluoro-6-methyl-1-(p-tolylsulfonyl)pyrrolo[2,3-b]pyridine-2-carboxylate (800 mg, 2.00 mmol, 25.83% yield, 95% purity) as a yellow solid.

LCMS (ESI) m/z 381.0 [M+H]⁺; ¹H NMR was recorded.

### Step 6. Synthesis of methyl 4,5-difluoro-6-methyl-1H-pyrrolo[2,3-b]pyridine-2-carboxylate

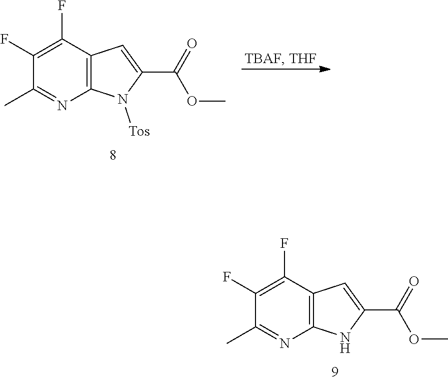

To a solution of methyl 4,5-difluoro-6-methyl-1-(p-tolylsulfonyl)pyrrolo[2,3-b]pyridine-2-carboxylate (1 g, 2.63 mmol, 1 eq) in THE (5 mL) was added TBAF (1 M in THF, 3.94 mL, 1.5 eq). The mixture was stirred at 25° C. for 0.5 hr. TLC indicated the reactant was consumed completely. The reaction mixture was concentrated under reduced pressure. The resulting residue was triturated with water (20 mL) for 20 min and filtered. The cake was collected, washed with petroleum ether (10 mL×3), concentrated under reduced pressure. Compound methyl 4,5-difluoro-6-methyl-1H-pyrrolo[2,3-b]pyridine-2-carboxylate (620 mg, 2.19 mmol, 83.41% yield, 80% purity) was obtained as a yellow solid. The crude product was used for the next step without further purification.

LCMS (ESI) m/z 227.0 [M+H]⁺; ¹H NMR was recorded.

### Synthesis of 4,5-difluoro-6-methyl-1H-pyrrolo[2,3-b]pyridine-2-carboxylic acid

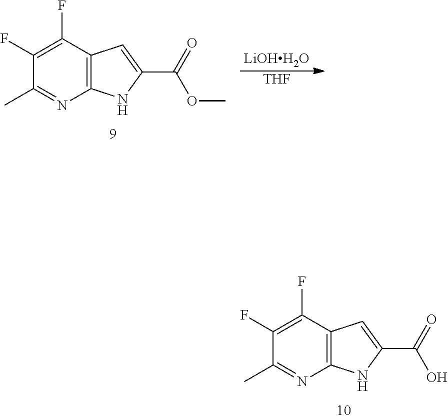

To a solution of methyl 4,5-difluoro-6-methyl-1H-pyrrolo[2,3-b]pyridine-2-carboxylate (620 mg, 2.74 mmol, 1 eq) in THE (3 mL) and H₂O (3 mL) was added LiOH.H₂O (575.15 mg, 13.71 mmol, 5 eq). The mixture was stirred at 25° C. for 12 hr. LC-MS showed the desired product was detected. The reaction mixture was concentrated under reduced pressure to remove THF. The residue was diluted with water (10 mL), aq. HCl (6M) was added until pH to 2. The mixture was filtered and concentrated in vacuo. Compound 4,5-difluoro-6-methyl-1H-pyrrolo[2,3-b]pyridine-2-carboxylic acid (500 mg, 2.12 mmol, 77.38% yield, 90% purity) was obtained as a yellow solid, which was used for the next step without further purification.

LCMS (ESI) m/z 212.8 [M+H]⁺; 1H NMR (500 MHz, DMSO-d6) δ=13.38 (br s, 1H), 12.64 (br s, 1H), 7.14 (d, J=2.0 Hz, 1H), 2.55 (d, J=3.5 Hz, 3H).

### Synthesis of N-(1,1-dimethylsilepan-4-yl)-4,5-difluoro-6-methyl-1H-pyrrolo[2,3-b]pyridine-2-carboxamide, N-[(4R)-1,1-dimethylsilepan-4-yl]-4,5-difluoro-6-methyl-1H-pyrrolo[2,3-b]pyridine-2-carboxamide and N-[(4S)-1,1-dimethylsilepan-4-yl]-4,5-difluoro-6-methyl-1H-pyrrolo[2,3-b]pyridine-2-carboxamide

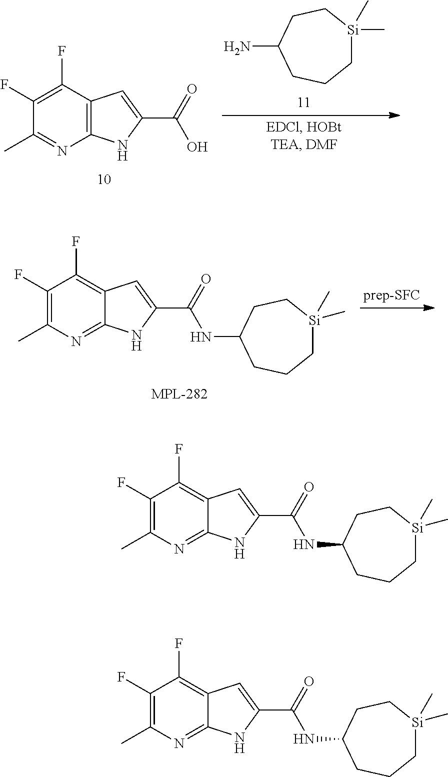

To a solution of 4,5-difluoro-6-methyl-1H-pyrrolo[2,3-b]pyridine-2-carboxylic acid (40 mg, 188.54 umol, 1 eq) and 1,1-dimethylsilepan-4-amine (43.85 mg, 226.25 umol, 1.2 eq, HCl) in DMF (1 mL) at 25° C. was added a solution of HOBt (76.43 mg, 565.63 umol, 3 eq) and EDCI (108.43 mg, 565.63 umol, 3 eq) in DMF (1 mL), followed by TEA (95.39 mg, 942.72 umol, 131.21 uL, 5 eq), and the mixture was stirred at 25° C. for 1 hr. LC-MS showed desired compound was detected. The reaction mixture was purified by prep-HPLC (column: Phenomenex Synergi C18 150*30 mm*4 um; mobile phase: A: 0.225% formic acid in water, B: CH3CN; gradient: 66%-95% B over 11 min). Compound N-(1,1-dimethylsilepan-4-yl)-4,5-difluoro-6-methyl-1H-pyrrolo[2,3-b]pyridine-2-carboxamide (29.7 mg, 82.95 umol, 43.99% yield, 98.16% purity) was obtained as a white solid.

LCMS (ESI) m/z 352.2 [M+H]⁺; ¹H NMR (400 MHz, DMSO-d6) δ=12.37 (br s, 1H), 8.32 (br d, J=7.8 Hz, 1H), 7.24 (s, 1H), 3.88 (br s, 1H), 2.53 (d, J=3.5 Hz, 3H), 1.92-1.47 (m, 6H), 0.81-0.57 (m, 4H), 0.04 (d, J=7.0 Hz, 6H).

The same reaction was conducted at 707.04 umol scale. The reaction mixture was purified by prep-HPLC (column: Phenomenex Synergi C18 150*30 mm*4 um; mobile phase: A: 0.225% formic acid in water, B: CH3CN; gradient: 60%-90% B over 11 min). The residue was further purified by SFC (Sepiatec Prep SFC 100, column: DAICEL CHIRALPAK AD (250 mm*30 mm, 10 um); mobile phase: A: 0.1% NH₃H₂O in MeOH, B CO₂, isocratic 60% B; Flow rate: 80 mL/min) to afford two peaks (two enantiomers), N-[(4R)-1,1-dimethylsilepan-4-yl]-4,5-difluoro-6-methyl-1H-pyrrolo[2,3-b]pyridine-2-carboxamide and N-[(4S)-1,1-dimethylsilepan-4-yl]-4,5-difluoro-6-methyl-1H-pyrrolo[2,3-b]pyridine-2-carboxamide.

Peak 1 (MPL-282A): 51.7 mg, 147.10 umol, 20.80% yield, 100% purity, white solid.

LCMS (ESI) m/z 352.2 [M+H]⁺; ¹H NMR (500 MHz, DMSO-d6) δ=12.36 (br s, 1H), 8.32 (d, J=7.9 Hz, 1H), 7.24 (s, 1H), 4.00-3.78 (m, 1H), 2.53 (d, J=3.4 Hz, 3H), 1.96-1.43 (m, 6H), 0.85-0.54 (m, 4H), 0.04 (d, J=8.9 Hz, 6H).

Peak 2 (MPL-282B): 50.2 mg, 142.83 umol, 20.20% yield, 100% purity, white solid. LCMS (ESI) m/z 352.3 [M+H]⁺; ¹H NMR (500 MHz, DMSO-d6) δ=12.35 (br s, 1H), 8.32 (d, J=7.9 Hz, 1H), 7.24 (s, 1H), 3.96-3.81 (m, 1H), 2.53 (d, J=3.5 Hz, 3H), 2.00-1.37 (m, 6H), 0.83-0.55 (m, 4H), 0.04 (d, J=8.9 Hz, 6H).

MPL-282A and MPL-282B were also analyzed by analytical SFC.

Conditions:

Instrument: CAS-SH-ANA-SFC-K (Waters UPCC with PDA Detector)
Column: Chiralpak AD-3 50*4.6 mm, 3 um particle size
Mobile phase: A: CO₂, B: 0.05% DEA in MeOH

### Isocratic: 40% B

Flow rate: 4 mL/min
Column temp: 35° C.

### ABPR: 1500 psi

MPL-282A: retention time 1.22 min; 100% ee; MPL-282B: retention time 1.93 min; 100% ee

### Example 143. MPL-284

### Synthesis of 4,5-difluoro-6-methyl-N-(5-silaspiro[4.5]decan-8-yl)-1H-pyrrolo[2,3-b]pyridine-2-carboxamide

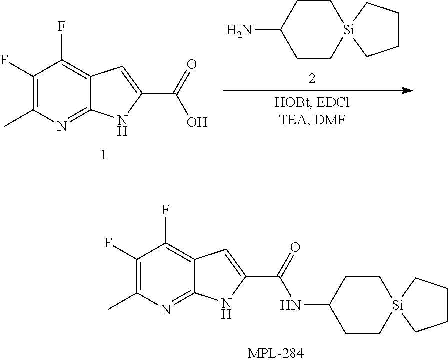 [SMILES: Cc1nc2[nH]c(C(=O)O)cc2c(F)c1F ; NC1CC[Si]2(CCCC2)CC1 ; Cc1nc2[nH]c(C(=O)NC3CC[Si]4(CCCC4)CC3)cc2c(F)c1F]

To a solution of 4,5-difluoro-6-methyl-1H-pyrrolo[2,3-b]pyridine-2-carboxylic acid (50 mg, 235.68 umol, 1 eq) and 5-silaspiro[4.5]decan-8-amine (50 mg, 242.95 umol, 1.03 eq, HCl salt) in DMF (5 mL) was added HOBt (95.54 mg, 707.04 umol, 3 eq) and EDCI (135.54 mg, 707.04 umol, 3 eq), followed by TEA (143.09 mg, 1.41 mmol, 196.82 uL, 6 eq). The mixture was stirred at 30° C. for 1 hr. LC-MS indicated desired product was detected. The reaction mixture was diluted with H₂O (30 mL) and extracted with EtOAc (30 mL×3). The combined organic layer was washed with saturated NaHCO₃ (30 mL×2) and 5% LiCl in water (30 mL×2), dried over Na₂SO₄, filtered and concentrated under reduced pressure. The resulting residue was purified by prep-HPLC (column: Phenomenex Synergi C18 150×30 mm×4 um; mobile phase: 0.05% HCl in water, B: CH3CN, gradient:70%-90% over 9 min) to afford 4,5-difluoro-6-methyl-N-(5-silaspiro[4.5]decan-8-yl)-1H-pyrrolo[2,3-b]pyridine-2-carboxamide (19.6 mg, 53.11 umol, 22.54% yield, 98.5% purity) as a white solid.

LCMS m/z: 364.1 [M+H]⁺; ¹H NMR (400 MHz, METHANOL-d₄) 8=7.14 (s, 1H), 3.81 (br t, J=11.3 Hz, 1H), 2.57 (d, J=3.5 Hz, 3H), 2.20 (br d, J=11.0 Hz, 2H), 1.72-1.54 (m, 6H), 0.91-0.80 (m, 4H), 0.67 (br t, J=6.7 Hz, 2H), 0.58 (br t, J=6.8 Hz, 2H).

### Example 144. MPL-285

### Synthesis of 4,5-difluoro-6-methyl-N-(6-silaspiro[5.5]undecan-3-yl)-1H-pyrrolo[2,3-b]pyridine-2-carboxamide

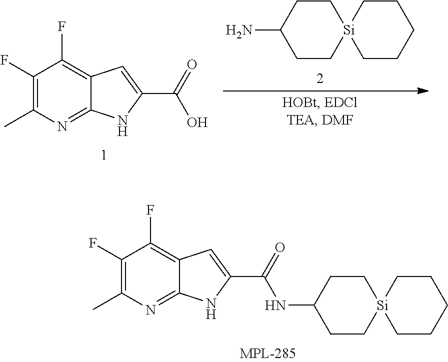 [SMILES: Cc1nc2[nH]c(C(=O)O)cc2c(F)c1F ; NC1CC[Si]2(CCCCC2)CC1 ; Cc1nc2[nH]c(C(=O)NC3CC[Si]4(CCCCC4)CC3)cc2c(F)c1F]

To a solution of 4,5-difluoro-6-methyl-1H-pyrrolo[2,3-b]pyridine-2-carboxylic acid (50 mg, 235.68 umol, 1 eq) and 6-silaspiro[5.5]undecan-3-amine (51.81 mg, 235.68 umol, 1 eq. HCl salt) in DMF (5 mL) was added HOBt (95.54 mg, 707.04 umol, 3 eq) and EDCI (135.54 mg, 707.04 umol, 3 eq), followed by TEA (143.09 mg, 1.41 mmol, 196.82 uL, 6 eq). The mixture was stirred at 30° C. for 1 hr. LC-MS indicated desired product was detected. The reaction mixture was diluted with H₂O (30 mL) and extracted with EtOAc 90 mL (30 mL×3). The combined organic layer was washed with saturated NaHCO₃ (30 mL×2) and 5% LiCl in water (30 mL×2), dried over Na₂SO₄, filtered and concentrated under reduced pressure. The resulting residue was purified by flash silica gel chromatography (ISCO®; 4 g SepaFlash® Silica Flash Column, eluent of 0-10% ethyl acetate in petroleum ether at 30 mL/min) to afford 4,5-difluoro-6-methyl-N-(6-silaspiro[5.5]undecan-3-yl)-1H-pyrrolo[2,3-b]pyridine-2-carboxamide (27.1 mg, 71.57 umol, 30.37% yield, 99.692% purity) as a white solid.

LCMS m/z: 378.1 [M+H]⁺; ¹H NMR (400 MHz, METHANOL-d₄) 8=7.14 (s, 1H), 3.78 (br t, J=11.3 Hz, 1H), 2.57 (d, J=3.5 Hz, 3H), 2.13 (br d, J=9.4 Hz, 2H), 1.80-1.59 (m, 6H), 1.45 (brd, J=5.1 Hz, 2H), 0.96 (br d, J=14.5 Hz, 2H), 0.82-0.73 (m, 2H), 0.71-0.61 (m, 4H).

### Example 145. MPL-290

### Synthesis of N-cyclooctyl-4,5-difluoro-6-methyl-1H-pyrrolo[2,3-b]pyridine-2-carboxamide

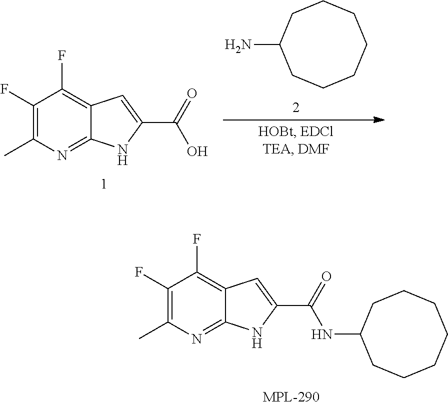 [SMILES: Cc1nc2[nH]c(C(=O)O)cc2c(F)c1F ; NC1CCCCCCC1 ; Cc1nc2[nH]c(C(=O)NC3CCCCCCC3)cc2c(F)c1F]

To a solution of 4,5-difluoro-6-methyl-1H-pyrrolo[2,3-b]pyridine-2-carboxylic acid (40 mg, 188.54 umol, 1 eq) and cyclooctanamine (23.99 mg, 188.54 umol, 1 eq) in DMF (0.5 mL) was added a solution of HOBt (76.43 mg, 565.63 umol, 3 eq) and EDCI (108.43 mg, 565.63 umol, 3 eq) in DMF (0.5 mL), followed by TEA (114.47 mg, 1.13 mmol, 157.46 uL, 6 eq). The mixture was stirred at 25° C. for 1 hr. LC-MS showed that desired compound was detected. The reaction mixture was filtered. The filtrate was purified by prep-HPLC (column: Phenomenex Synergi C18 150*30 mm*4 um; mobile phase: A: 0.225% formic acid in water, B: CH₃CN; gradient: 55%-85% B over 11 min). Compound N-cyclooctyl-4,5-difluoro-6-methyl-1H-pyrrolo[2,3-b]pyridine-2-carboxamide (20 mg, 61.70 umol, 32.72% yield, 99.14% purity) was obtained as a white solid.

LCMS m/z: 322.2 [M+1]⁺; ¹H NMR (400 MHz, METHANOL-d4) δ=7.17 (s, 1H), 4.20-4.12 (m, 1H), 2.58 (d, J=3.5 Hz, 3H), 1.93-1.85 (m, 2H), 1.83-1.74 (m, 4H), 1.71-1.59 (m, 8H).

### Example 146: MPL-292

### Synthesis of 5-chloro-N-(1,1-dimethylsilepan-4-yl)-4-fluoro-6-methyl-1H-pyrrolo[2,3-b]pyridine-2-carboxamide

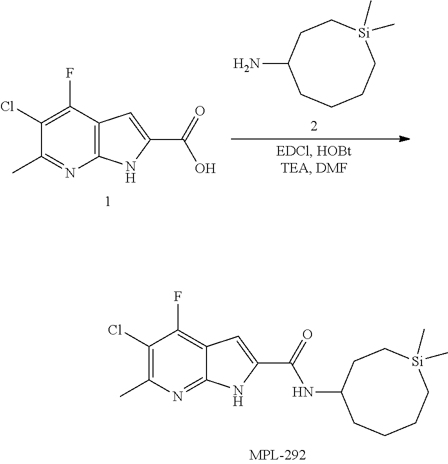

To a solution of 5-chloro-4-fluoro-6-methyl-1H-pyrrolo[2,3-b]pyridine-2-carboxylic acid (30 mg, 131.23 umol, 1 eq) and 1,1-dimethylsilocan-4-amine (30.00 mg, 144.35 umol, 1.1 eq, HCl salt) in DMF (1 mL) at 25° C. was added a solution of EDCI (75.47 mg, 393.69 umol, 3 eq) and HOBt (53.20 mg, 393.69 umol, 3 eq) in DMF (1 mL), followed by TEA (66.40 mg, 656.15 umol, 91.33 uL, 5 eq). The reaction mixture was stirred at 25° C. for 2 hrs. LC-MS showed desired compound was detected. The mixture was purified by prep-HPLC (column: YMC-Actus Triart C18 150*30 mm*5 um; mobile phase: A: 0.225% formic acid in water; B: CH₃CN; gradient: 75%-100% B over 11 min). Compound 5-chloro-N-(1,1-dimethylsilocan-4-yl)-4-fluoro-6-methyl-1H-pyrrolo[2,3-b]pyridine-2-carboxamide (18.7 mg, 45.71 umol, 34.83% yield, 93.361% purity) was obtained as a white solid.

LCMS (ESI) m/z 382.1 [M+H]⁺; ¹H NMR (500 MHz, CHLOROFORM-d) δ=9.41 (br s, 1H), 6.78 (d, J=2.0 Hz, 1H), 6.08 (br d, J=7.8 Hz, 1H), 4.26-4.05 (m, 1H), 2.72 (s, 3H), 2.11-1.97 (m, 1H), 1.78-1.64 (m, 5H), 1.64-1.58 (m, 2H), 0.88-0.58 (m, 4H), 0.12-0.02 (m, 6H).

### Example 147. MPL-294

### Synthesis of 4,5-difluoro-N-[(1R,2R,3S,5R)-2-hydroxy-2,6,6-trimethyl-norpinan-3-yl]-6-methyl-1H-pyrrolo[2,3-b]pyridine-2-carboxamide

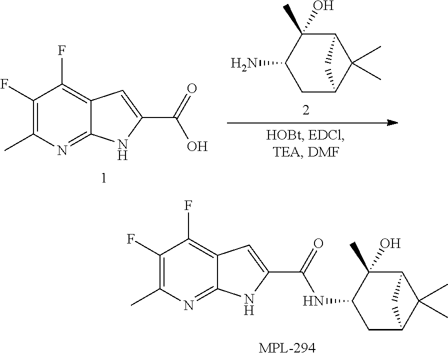

To a solution of 4,5-difluoro-6-methyl-1H-pyrrolo[2,3-b]pyridine-2-carboxylic acid (50 mg, 235.68 umol, 1 eq) and (1R,2R,3S,5R)-3-amino-2,6,6-trimethyl-norpinan-2-ol (50 mg, 243.04 umol, 1.03 eq, HCl) in DMF (2 mL) was added HOBt (95.53 mg, 707.04 umol, 3 eq) and EDCI (135.54 mg, 707.04 umol, 3 eq), followed by TEA (143.09 mg, 1.41 mmol, 196.82 uL, 6 eq). The mixture was stirred at 25° C. for 1 hr. The reaction mixture was purified by prep-HPLC (column: Phenomenex Synergi C18 150×30 mm×4 um; mobile phase A: 0.05% HCl in water, B: CH3CN, gradient: 65%-85% B over 9 min) to afford a white solid (50 mg), which was further purified by prep-SFC (column: DAICEL CHIRALPAK AD-H (250 mm×30 mm, 5 um); mobile phase: A: 0.1% NH₃H₂O in IPA, B: CO₂; isocratic 25% B, flow rate: 80 mL/min) and followed by lyophilization to give the desired compound 4,5-difluoro-N-[(1R,2R,3S,5R)-2-hydroxy-2,6,6-trimethyl-norpinan-3-yl]-6-methyl-1H-pyrrolo[2,3-b]pyridine-2-carboxamide (16 mg, 44.03 umol, 32.00% yield, 100% purity) as a white solid.

LCMS m/z: 364.1 [M+H]⁺; ¹H NMR (400 MHz, METHANOL-d₄) δ=7.27-7.15 (m, 1H), 4.70-4.60 (m, 1H), 2.61 (d, J=3.5 Hz, 3H), 2.55-2.46 (m, 1H), 2.32-2.23 (m, 1H), 2.08-2.02 (m, 1H), 2.01-1.96 (m, 1H), 1.73 (ddd, J=1.8, 7.5, 13.6 Hz, 1H), 1.66 (d, J=10.4 Hz, 1H), 1.35 (d, J=7.8 Hz, 6H), 1.16 (s, 3H).

### Example 148. MPL-295, MPL-295A and MPL-295B

### Synthesis of N-(1,1-dimethylsilolan-3-yl)-4,5-difluoro-6-methyl-1H-pyrrolo[2,3-b]pyridine-2-carboxamide, N-[(3R)-1,1-dimethylsilolan-3-yl]-4,5-difluoro-6-methyl-1H-pyrrolo[2,3-b]pyridine-2-carboxamide and N-[(3S)-1,1-dimethylsilolan-3-yl]-4,5-difluoro-6-methyl-1H-pyrrolo[2,3-b]pyridine-2-carboxamide

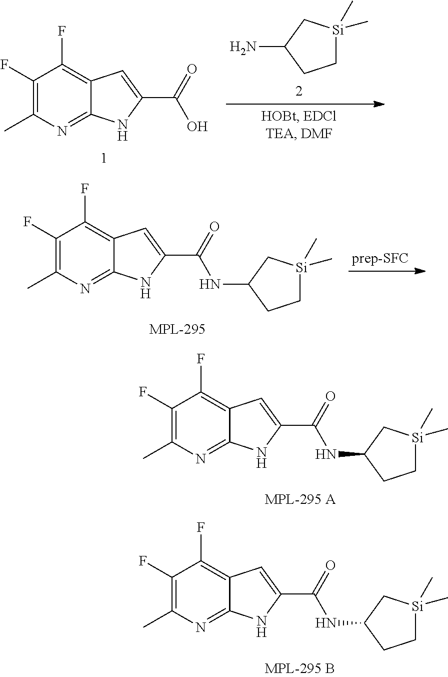

To a solution of 4,5-difluoro-6-methyl-1H-pyrrolo[2,3-b]pyridine-2-carboxylic acid (30 mg, 141.41 umol, 1 eq) and 1,1-dimethylsilolan-3-amine (23.44 mg, 141.41 umol, 1 eq, HCl salt) in DMF (2 mL) was added HOBt (57.32 mg, 424.22 umol, 3 eq) and EDCI (81.32 mg, 424.22 umol, 3 eq), followed by TEA (85.85 mg, 848.45 umol, 118.09 uL, 6 eq). The mixture was stirred at 25° C. for 2 hr. LC-MS indicated desired compound was detected. MeOH (0.5 mL) was added and the mixture was purified by prep-HPLC (column: Phenomenex Synergi C18 150×30 mm×4 um; mobile phase: A: 0.05% HCl in water, B: CH₃CN; gradient: 60%-80% B over 9 min). Compound N-(1,1-dimethylsilolan-3-yl)-4,5-difluoro-6-methyl-1H-pyrrolo[2,3-b]pyridine-2-carboxamide (17.6 mg, 53.19 umol, 37.62% yield, 97.743% purity) was obtained as a white solid.

LCMS m/z: 324.1 [M+H]⁺; ¹H NMR (400 MHz, METHANOL-d₄) 8=7.00 (s, 1H), 3.97-3.82 (m, 1H), 2.41 (d, J=3.4 Hz, 3H), 2.08-1.88 (m, 1H), 1.29 (dq, J=7.2, 12.2 Hz, 1H), 1.10-0.99 (m, 1H), 0.69 (dd, J=6.5, 14.3 Hz, 1H), 0.51-0.35 (m, 2H), 0.00 (d, J=1.7 Hz, 6H).

The above reaction was later conducted at 471.36 umol. The product isolated from prep-HPLC (column: Phenomenex Synergi C18 100*21.2 mm*4 um; mobile phase: A: 0.225% formic acid in water; B: CH₃CN; gradient: 52%-82% B over 11 min) was further by SFC (Berger MG II, column: DAICEL CHIRALPAK AD (250 mm*30 mm, 10 um); mobile phase: A: 0.1% NH₃H₂O in EtOH; B: CO₂; isocratic 50% B; flow rate: 80 mL/min) to give two peaks (two enantiomers), N-[(3R)-1,1-dimethylsilolan-3-yl]-4,5-difluoro-6-methyl-1H-pyrrolo[2,3-b]pyridine-2-carboxamide and N-[(3S)-1,1-dimethylsilolan-3-yl]-4,5-difluoro-6-methyl-1H-pyrrolo[2,3-b]pyridine-2-carboxamide.

Peak 1 (MPL-295A): 33.4 mg, 103.27 umol, 21.91% yield, 100% purity.

LCMS (ESI) m/z 324.1 [M+H]⁺; ¹H NMR (500 MHz, DMSO-d6) δ=12.36 (br s, 1H), 8.35 (d, J=7.6 Hz, 1H), 7.22 (s, 1H), 4.02 (dq, J=7.0, 11.5 Hz, 1H), 2.53 (d, J=3.4 Hz, 3H), 2.10-1.97 (m, 1H), 1.44 (dq, J=7.2, 12.0 Hz, 1H), 1.17-1.06 (m, 1H), 0.81 (ddd, J=1.8, 7.1, 14.6 Hz, 1H), 0.69-0.46 (m, 2H), 0.18 (d, J=2.7 Hz, 6H).

Peak 2 (MLL-295B): 32.5 mg, 100.49 umol, 21.32% yield, 100% purity.

LCMS (ESI) m/z 324.1 [M+H]⁺; ¹H NMR (500 MHz, DMSO-d6) δ=12.36 (br s, 1H), 8.35 (d, J=7.6 Hz, 1H), 7.22 (s, 1H), 4.08-3.94 (m, 1H), 2.53 (d, J=3.4 Hz, 3H), 2.08-2.00 (m, 1H), 1.50-1.38 (m, 1H), 1.17-1.06 (m, 1H), 0.86-0.77 (m, 1H), 0.68-0.48 (m, 2H), 0.18 (d, J=2.7 Hz, 6H).

MPL-295A and MPL-295B was also analyzed by analytical SFC.

### Conditions:

Instrument: CAS-SH-ANA-SFC-L (Waters UPCC with PDA Detector)
Column: Chiralpak AD-3 150 mm*4.6 mm, 3 um particle size
Mobile phase: A: CO₂, B: 0.05% DEA in ethanol
Gradient: 5% to 40% of B in 5 min and hold 40% B for 2.5 min, then 5% of B for 2.5 min
Flow rate: 2.5 mL/min
Column temp.: 35° C.

### ABPR: 1500 psi

MPL-295A: retention time: 6.19 min; 100% ee; MPL-295B: retention time: 7.250 min, 100% ee

### Example 149. MPL-301

### Synthesis of N-(1,1-dimethylsilocan-5-yl)-4,5-difluoro-6-methyl-1H-pyrrolo[2,3-b]pyridine-2-carboxamide

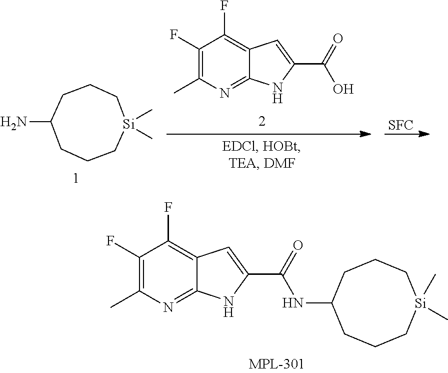

To a solution of 4,5-difluoro-6-methyl-1H-pyrrolo[2,3-b]pyridine-2-carboxylic acid (40 mg, 188.54 umol, 1 eq) and 1,1-dimethylsilocan-5-amine (35.26 mg, 169.69 umol, 0.9 eq, HCl salt) in DMF (1 mL) was added a solution of EDCI (72.29 mg, 377.09 umol, 2 eq) and HOBt (50.95 mg, 377.09 umol, 2 eq) in DMF (0.5 mL), followed by TEA (76.31 mg, 754.17 umol, 104.97 uL, 4 eq). The mixture was stirred at 20° C. for 2 hr. LC-MS showed one main peak with desired mass. The mixture was filtered to remove insoluble matter. The filtrate was purified by prep-HPLC (column: YMC-Actus Triart C18 150*30 mm*5 um; mobile phase: A 0.225% formic acid in water, B: CH₃CN; gradient: 70%-100% B over 11 min). The resulting residue was further purified by SFC (Berger MG II, column: DAICEL CHIRALPAK AD (250 mm*30 mm, 10 um); mobile phase column: 0.1% NH₃H₂O in EtOH, B: CO₂; isocratic 40% B, flow rate 80 mL/min). Compound N-(1,1-dimethylsilocan-5-yl)-4,5-difluoro-6-methyl-1H-pyrrolo[2,3-b]pyridine-2-carboxamide (28.2 mg, 76.76 umol, 40.71% yield, 99.49% purity) was obtained as a white solid.

LCMS (ESI) m/z 366.1 [M+H]⁺; ¹H NMR (500 MHz, Acetone-d6)=11.18 (br s, 1H), 7.75 (br d, J=7.2 Hz, 1H), 7.25-6.99 (m, 1H), 4.42-3.97 (m, 1H), 2.55 (d, J=3.5 Hz, 3H), 1.83-1.65 (m, 8H), 0.80 (t, J=6.2 Hz, 4H), 0.11-−0.03 (m, 6H).

### Example 150. MPL-305 and MPL-460

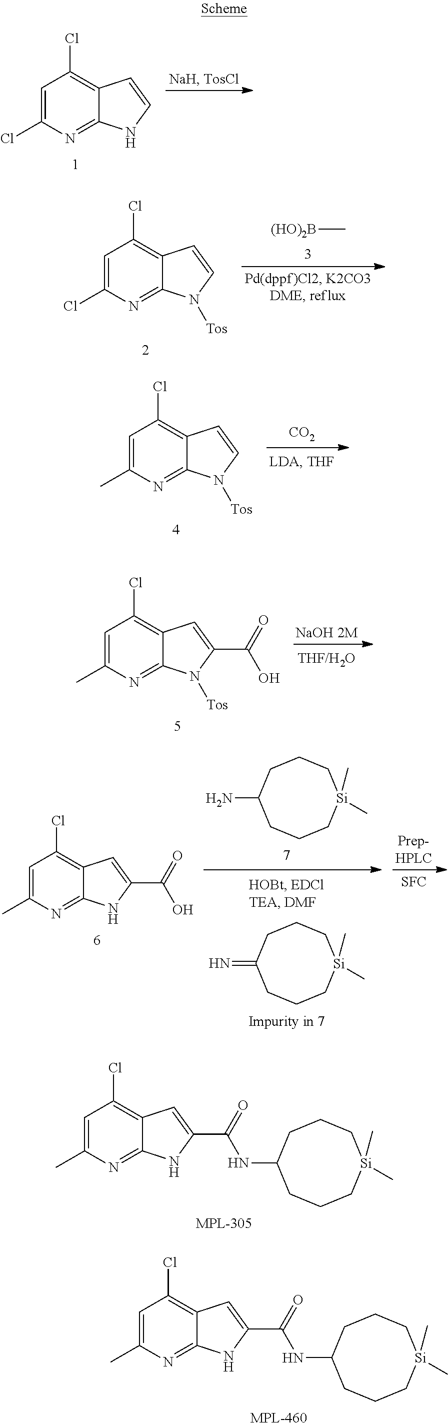

### Synthesis of 4,6-dichloro-1-(p-tolylsulfonyl)pyrrolo[2,3-b]pyridine

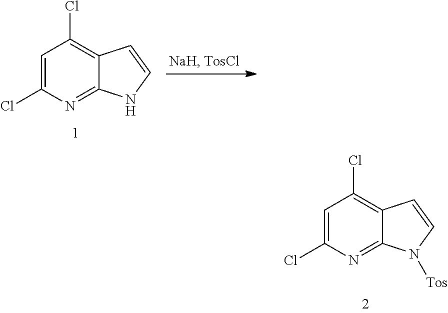

To a solution of 4,6-dichloro-1H-pyrrolo[2,3-b]pyridine (5 g, 26.73 mmol, 1 eq) in THE (100 mL) was added NaH (1.60 g, 40.10 mmol, 60% purity, 1.5 eq) in batches at 0° C. The mixture was stirred at 0° C. for 1 hr. 4-methylbenzenesulfonyl chloride (6.12 g, 32.08 mmol, 1.2 eq) was added at 0° C. The reaction mixture was stirred at 15° C. for 1 hr. TLC (petroleum ether:EtOAc=10:1) showed starting material was consumed completely, and one major spot formed. The mixture was poured into saturated NH₄Cl (100 mL) and extracted with EtOAc (50 mL×3). The combined organic layer was washed with brine (50 mL×2), filtered and concentrated under reduced pressure. The resulting residue was purified by flash silica gel chromatography (ISCO®; 40 g SepaFlash® Silica Flash Column, eluent of 0˜30% ethyl acetate in petroleum ether at 40 mL/min). Compound 4,6-dichloro-1-(p-tolylsulfonyl)pyrrolo[2,3-b]pyridine (7.5 g, 21.54 mmol, 80.57% yield, 98% purity) was obtained as a white solid. ¹H NMR was recorded.

### Synthesis of 4-chloro-6-methyl-1-(p-tolylsulfonyl)pyrrolo[2,3-b]pyridine

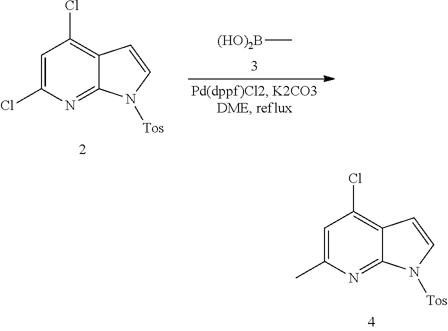

To a mixture of 4,6-dichloro-1-(p-tolylsulfonyl)pyrrolo[2,3-b]pyridine (7 g, 20.52 mmol, 1 eq), methylboronic acid (1.72 g, 28.72 mmol, 1.4 eq) and K₂CO₃ (8.51 g, 61.55 mmol, 3 eq) in DME (300 mL) was added Pd(dppf)Cl₂.CH₂Cl₂ (1.68 g, 2.05 mmol, 0.1 eq) under N₂. The mixture was stirred and refluxed at 120° C. (outside temperature) for 12 hr. TLC (petroleum ether. EtOAc=10:1) showed starting material was consumed completely and two major spots formed. The mixture was filtered, the filtrate was concentrated under reduced pressure. The resulting residue was purified by flash silica gel chromatography (ISCO®; 120 g SepaFlash® Silica Flash Column, Eluent of 0˜5% ethyl acetate in petroleum ether at 85 mL/min). Compound 4-chloro-6-methyl-1-(p-tolylsulfonyl)pyrrolo[2,3-b]pyridine (4.3 g, 12.73 mmol, 62.07% yield, 95% purity) was obtained as a white solid. ¹H NMR was recorded.

### Synthesis of 4-chloro-6-methyl-1-(p-tolylsulfonyl)pyrrolo[2,3-b]pyridine-2-carboxylic acid

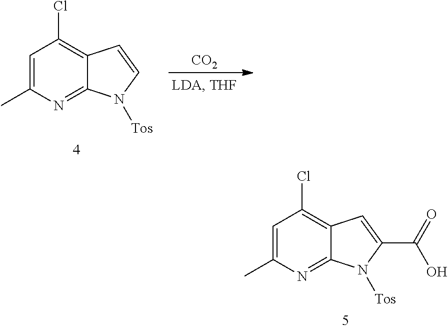

To a solution of 4-chloro-6-methyl-1-(p-tolylsulfonyl)pyrrolo[2,3-b]pyridine (0.5 g, 1.56 mmol, 1 eq) in THE (10 mL) (dried by Na, and distilled) was added LDA (2 M in THF, 1.17 mL, 1.5 eq) under N₂ at −78° C. dropwise. The mixture was stirred at −78° C. for 1.5 hr. The reaction mixture was then purged with CO₂ for 3 times, and was allowed to warm to 10° C. gradually and stirred under CO₂ for 2 hr. LC-MS showed one main peak with desired mass. Compound 4-chloro-6-methyl-1-(p-tolylsulfonyl)pyrrolo[2,3-b]pyridine-2-carboxylic acid (568 mg, crude) was obtained as a white solid suspended in THF.

### Synthesis of 4-chloro-6-methyl-1H-pyrrolo[2,3-b]pyridine-2-carboxylic acid

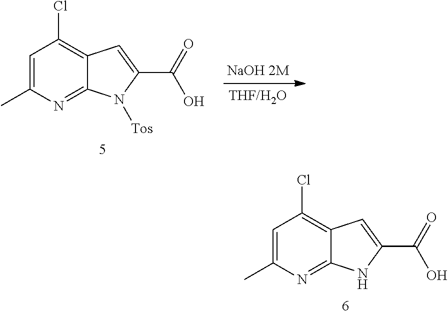

To a solution of 4-chloro-6-methyl-1-(p-tolylsulfonyl)pyrrolo[2,3-b]pyridine-2-carboxylic acid (568 mg, crude, 1.56 mmol, 1 eq) in THE was added NaOH (2 M, 18.93 mL, 24.32 eq). The mixture was stirred at 50° C. for 2 hr. LCMS showed starting material was consumed completely and desired product was detected. The reaction mixture was extracted with EtOAc (10 mL×2). The pH of aqueous phase was adjusted to 5 by HCl (6M in water), and then extracted with EtOAc (10 mL×3). The combined organic layer was dried over Na₂SO₄, filtered and concentrated under reduced pressure to afford 4-chloro-6-methyl-1H-pyrrolo[2,3-b]pyridine-2-carboxylic acid (210 mg, 947.22 umol, 60.84% yield, 95% purity) as a light yellow solid.

LCMS (ESI) m/z 210.9 [M+H]⁺; ¹H NMR (500 MHz, DMSO-d6) δ=12.51 (br s, 1H), 7.21 (s, 1H), 7.01 (s, 1H), 2.54 (s, 3H).

### Synthesis of 4-chloro-N-(1,1-dimethylsilocan-5-yl)-6-methyl-1H-pyrrolo[2,3-b]pyridine-2-carboxamide and 4-chloro-N-(1,1-dimethylsilocan-5-ylidene)-6-methyl-1H-pyrrolo[2,3-b]pyridine-2-carboxamide

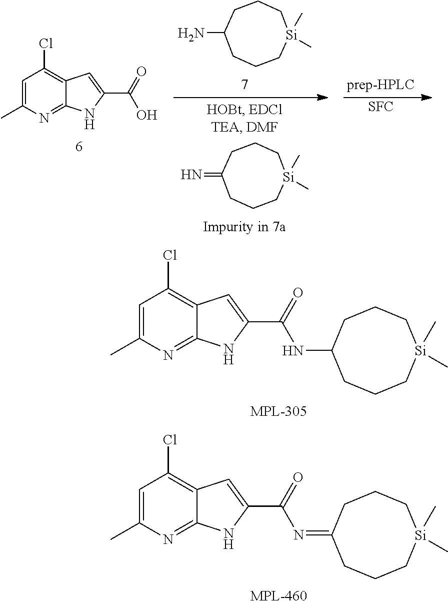

To a solution of 4-chloro-6-methyl-1H-pyrrolo[2,3-b]pyridine-2-carboxylic acid (27.87 mg, 132.33 umol, 1.1 eq) and 1,1-dimethylsilocan-5-amine (25 mg, 120.30 umol, 1 eq, HCl salt)(containing 7a as impurity) in DMF (1 mL) was added a solution of EDCI (46.12 mg, 240.60 umol, 2 eq) and HOBt (32.51 mg, 240.60 umol, 2 eq) in DMF (1 mL), followed by TEA (48.69 mg, 481.20 umol, 66.98 uL, 4 eq). The mixture was stirred at 20° C. for 2 hr. LC-MS showed the reactant was consumed completely and one main peak with desired mass was detected. The reaction mixture was filtered to remove insoluble matter. The filtrate was purified by prep-HPLC (column: YMC-Actus Triart C18 150*30 mm*5 um; mobile phase: A: 0.225% formic acid in water; B: CH3CN; gradient: 70%-100% B over 11 min). SFC showed more than 1 peak. The residue from prep-HPLC was further purified by SFC (Sepiatec Prep SFC 100; column: DAICEL CHIRALPAK AD (250 mm*30 mm, 10 um particle size); mobile phase: A: 0.1% NH₃H₂O in EtOH, B: CO₂; isocratic 40% B, flow rate: 80 mL/min). Compound 4-chloro-N-(1,1-dimethylsilocan-5-yl)-6-methyl-1H-pyrrolo[2,3-b]pyridine-2-carboxamide (18 mg, 49.46 umol, 41.11% yield, 100% purity) was obtained as a white solid.

LCMS (ESI) m/z 364.1 [M+H]⁺; ¹H NMR (500 MHz, DMSO-d₆) δ=12.18 (s, 1H), 8.35 (d, J=8.1 Hz, 1H), 7.13 (d, J=2.0 Hz, 1H), 7.10 (s, 1H), 3.98 (br d, J=6.1 Hz, 1H), 2.46 (s, 3H), 1.67-1.50 (m, 8H), 0.74-0.58 (m, 4H), 0.00 (s, 3H), −0.05 (s, 3H).

The same reaction was conducted at 427.32 umol scale. The residue from prep-HPLC purification was further purified by SFC. Compound 4-chloro-N-(1,1-dimethylsilocan-5-ylidene)-6-methyl-1H-pyrrolo[2,3-b]pyridine-2-carboxamide (7.4 mg, 20.35 umol, 36.83% yield, 99.5% purity) was obtained as a white solid.

LCMS m/z 362.1 [M+1]⁺; ¹H NMR (400 MHz, METHANOL-d4) 6=7.16 (s, 1H), 7.07 (s, 1H), 2.76-2.64 (m, 2H), 2.60 (s, 3H), 2.53-2.46 (m, 1H), 2.28 (br dd, J=2.6, 14.0 Hz, 1H), 1.89-1.80 (m, 2H), 1.62-1.40 (m, 2H), 0.92-0.82 (m, 2H), 0.80-0.66 (m, 2H), 0.07 (s, 3H), 0.02 (s, 3H).

### Example 151. MPL-316

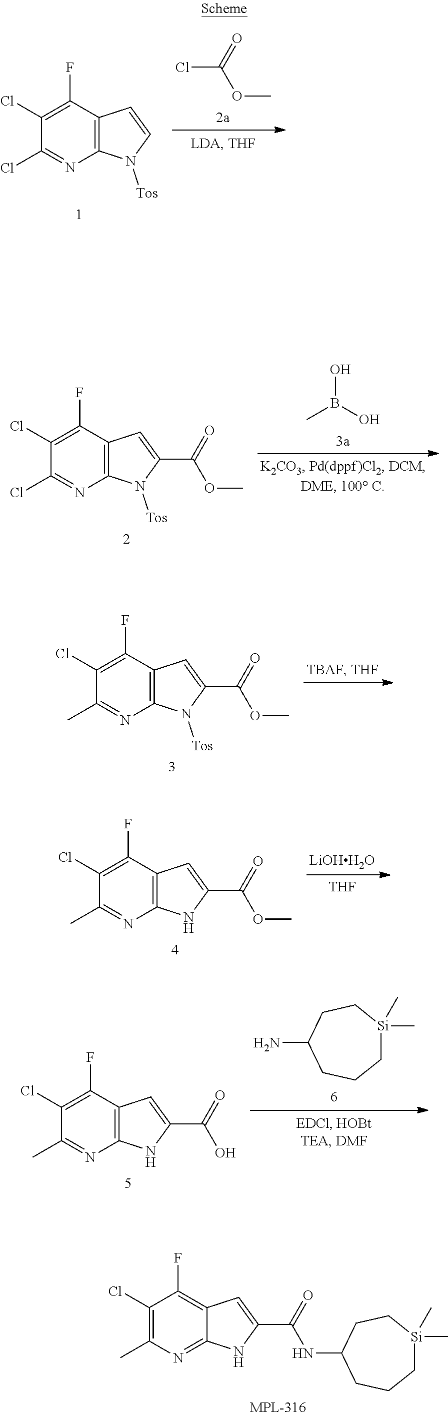

### Synthesis of methyl 5,6-dichloro-4-fluoro-1-(p-tolylsulfonyl)pyrrolo[2,3-b]pyridine-2-carboxylate

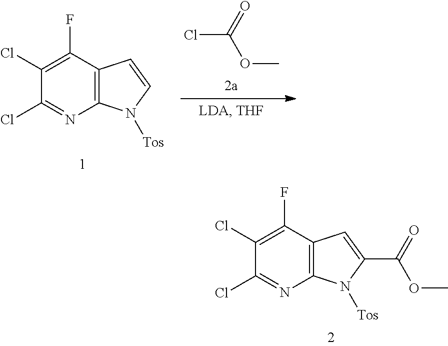

A mixture of 5,6-dichloro-4-fluoro-1-(p-tolylsulfonyl)pyrrolo[2,3-b]pyridine (3.9 g, 10.86 mmol, 1 eq) in THE (40 mL) was degassed and purged with N₂ for 3 times, then LDA (2 M in THF, 8.14 mL, 1.5 eq) was added and stirred at −60° C. for 10 min under N₂ atmosphere. Methyl carbonochloridate (5.13 g, 54.29 mmol, 4.20 mL, 5 eq) was then added. The mixture was stirred at −60° C. for 30 min. TLC showed one major new spot. The reaction mixture was quenched with saturated NH₄Cl solution (100 mL) at 25° C., and then diluted with water (50 mL) and extracted with EtOAc (100 mL×2). The combined organic layer was washed with brine (100 mL×2), dried over Na₂SO₄, filtered and concentrated under reduced pressure. The resulting residue was purified by column chromatography (SiO₂, petroleum ether/ethyl acetate=1/0 to 5/1). Compound methyl 5,6-dichloro-4-fluoro-1-(p-tolylsulfonyl)pyrrolo[2,3-b]pyridine-2-carboxylate (1.1 g, 2.11 mmol, 19.43% yield, 80% purity) was obtained as a yellow solid. ¹H NMR was recorded.

### Synthesis of methyl 5-chloro-4-fluoro-6-methyl-1-(p-tolylsulfonyl)pyrrolo[2,3-b]pyridine-2-carboxylate

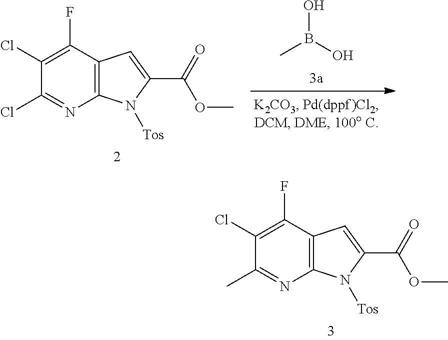

To a mixture of methyl 5,6-dichloro-4-fluoro-1-(p-tolylsulfonyl)pyrrolo[2,3-b]pyridine-2-carboxylate (1.1 g, 2.64 mmol, 1 eq), methylboronic acid (205.16 mg, 3.43 mmol, 1.3 eq) and K₂CO₃ (728.73 mg, 5.27 mmol, 2 eq) was added DME (5 mL). The mixture was purged with N₂ and Pd(dppf)Cl₂.CH₂Cl₂ (215.30 mg, 263.64 umol, 0.1 eq) was added under N₂. The mixture was stirred at 100° C. for 12 hr. LC-MS showed desired compound was detected. The reaction mixture was filtered, the cake was washed with EtOAc (20 mL×3). The combined filtrate was dried over Na₂SO₄ and concentrated under reduced pressure. The residue was purified by column chromatography (SiO₂, petroleum ether/ethyl acetate=1/0 to 10/1) to afford methyl 5-chloro-4-fluoro-6-methyl-1-(p-tolylsulfonyl)pyrrolo[2,3-b]pyridine-2-carboxylate (553 mg, 1.25 mmol, 47.57% yield, 90% purity) as a yellow solid. ¹H NMR was recorded.

### Synthesis of methyl 5-chloro-4-fluoro-6-methyl-1H-pyrrolo[2,3-b]pyridine-2-carboxylate

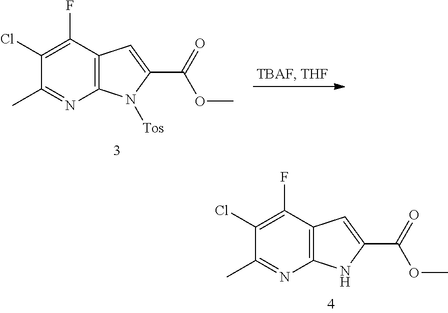

To a solution of methyl 5-chloro-4-fluoro-6-methyl-1-(p-tolylsulfonyl)pyrrolo[2,3-b]pyridine-2-carboxylate (553 mg, 1.39 mmol, 1 eq) in THE (5 mL) was added TBAF (1 M in THF, 1.81 mL, 1.3 eq). The mixture was stirred at 25° C. for 30 min. TLC indicated the reactant was consumed completely. LCMS showed desired compound was detected. The reaction mixture was concentrated under reduced pressure. The residue was triturated with water (10 mL) at 25° C. for 30 min, filtered, and the cake was collected. Compound methyl 5-chloro-4-fluoro-6-methyl-1H-pyrrolo[2,3-b]pyridine-2-carboxylate (446 mg, crude) was obtained as a yellow solid. ¹H NMR was recorded.

### Synthesis of 5-chloro-4-fluoro-6-methyl-1H-pyrrolo[2,3-b]pyridine-2-carboxylic acid

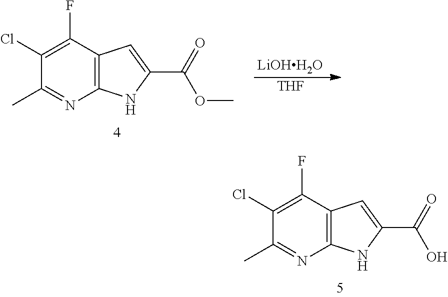

To a solution of methyl 5-chloro-4-fluoro-6-methyl-1H-pyrrolo[2,3-b]pyridine-2-carboxylate (446 mg, 1.84 mmol, 1 eq) in THE (3 mL) was added a solution of LiOH.H₂O (462.78 mg, 11.03 mmol, 6 eq) in H₂O (3 mL). The mixture was stirred at 30° C. for 12 hr. TLC indicated the reaction was completed. The reaction mixture was concentrated under reduced pressure to remove THF. The aqueous phase was adjusted to pH 2 with aqueous HCl (6 M), and then filtered and concentrated under reduced pressure to afford 5-chloro-4-fluoro-6-methyl-1H-pyrrolo[2,3-b]pyridine-2-carboxylic acid (337 mg, 1.33 mmol, 72.18% yield, 90% purity) as a yellow solid. ¹H NMR was recorded. The crude product was used directly for next step without further purification.

### Synthesis of 5-chloro-N-(1,1-dimethylsilepan-4-yl)-4-fluoro-6-methyl-1H-pyrrolo[2,3-b]pyridine-2-carboxamide

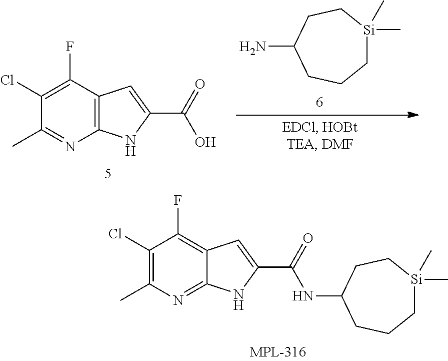

To a solution of 5-chloro-4-fluoro-6-methyl-1H-pyrrolo[2,3-b]pyridine-2-carboxylic acid (100 mg, 437.43 umol, 1 eq) and 1,1-dimethylsilepan-4-amine (75.70 mg, 390.64 umol, 8.93e-1 eq, HCl salt) in DMF (2 mL) at 25° C. was added a solution of HOBt (177.32 mg, 1.31 mmol, 3 eq) and EDCI (251.57 mg, 1.31 mmol, 3 eq) in DMF (2 mL) with stirring, followed by TEA (221.32 mg, 2.19 mmol, 304.42 uL, 5 eq). The reaction mixture was stirred at 25° C. for 2 hr. LC-MS showed desired compound was detected. The mixture was purified by prep-HPLC (column: YMC-Actus Triart C18 150*30 mm*5 um; mobile phase: A: 0.225% formic acid in water; B: CH₃CN; gradient: 70%-100% B over 11 min) to afford 5-chloro-N-(1,1-dimethylsilepan-4-yl)-4-fluoro-6-methyl-1H-pyrrolo[2,3-b]pyridine-2-carboxamide (59.4 mg, 152.33 umol, 34.82% yield, 94.352% purity) as a white solid.

LCMS (ESI) m/z 368.1 [M+H]⁺; ¹H NMR (500 MHz, CHLOROFORM-d) δ=9.37 (br s, 1H), 6.92-6.63 (m, 1H), 6.09 (br d, J=7.5 Hz, 1H), 4.06 (br d, J=8.5 Hz, 1H), 2.72 (s, 3H), 2.22-1.97 (m, 2H), 1.92-1.74 (m, 1H), 1.73-1.65 (m, 1H), 1.61 (br s, 1H), 1.51-1.41 (m, 1H), 0.87-0.59 (m, 4H), 0.06 (d, J=2.0 Hz, 6H).

### Example 152. MPL-316, MPL-316A and MPL-316B

MPL-316 was also made via different route described in the scheme below and its enantiomers MPL-316A and MPL-316B were obtained after SFC purification.

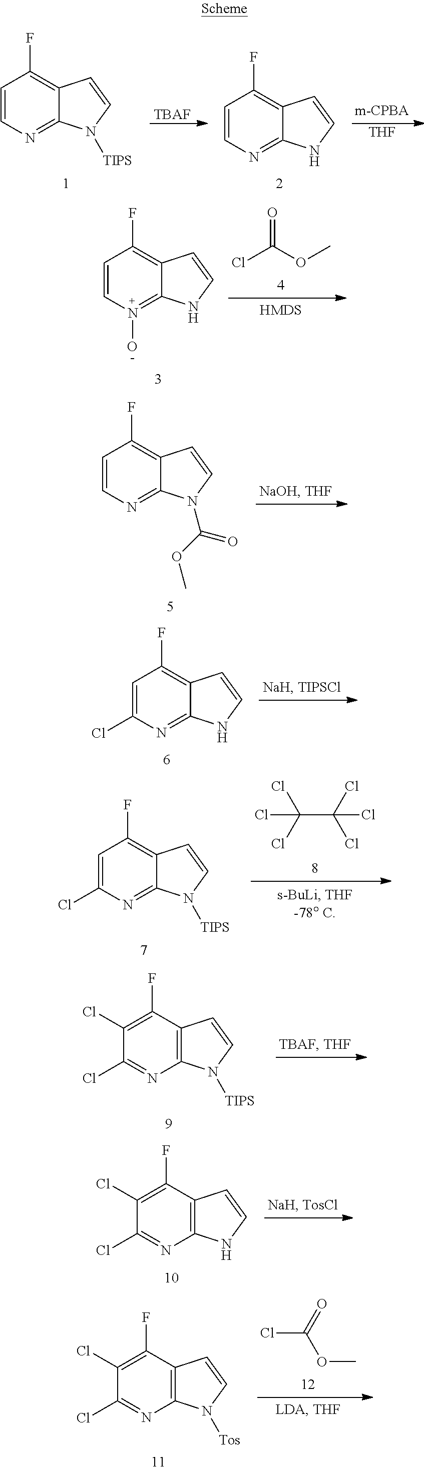

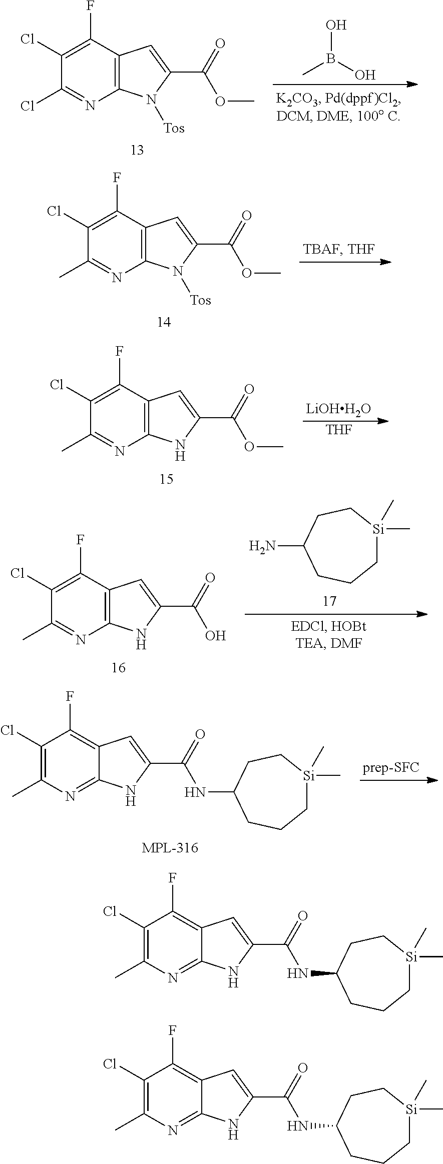

### Synthesis of 4-fluoro-1H-pyrrolo[2,3-b]pyridine

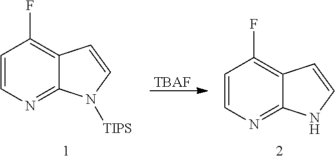

To a solution of (4-fluoropyrrolo[2,3-b]pyridin-1-yl)-triisopropyl-silane (160 g, 547.07 mmol, 1 eq) in THE (300 mL) was added TBAF (1 M in THF, 601.78 mL, 1.1 eq). The mixture was stirred at 25° C. for 30 min. TLC indicated reactant 1 was consumed completely. The reaction mixture was concentrated under reduced pressure and then poured into water, cooled to 0° C., filtered. The cake was washed with EtOAc (100 mL×10). The combined aqueous layer was extracted with EtOAc (200 mL×5). The combined organic layer was dried over Na₂SO₄, concentrated under reduced pressure. The resulting residue was purified by column chromatography (SiO₂, petroleum ether/ethyl acetate=1/0 to 0/1). Compound 4-fluoro-1H-pyrrolo[2,3-b]pyridine (82 g, 512.02 mmol, 83.19% yield, 85% purity) was obtained as a pink solid. ¹H NMR was recorded.

### Synthesis of 4-fluoro-7-oxido-1H-pyrrolo[2,3-b]pyridin-7-ium

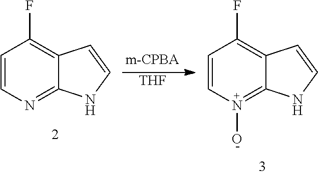

To a solution of 4-fluoro-1H-pyrrolo[2,3-b]pyridine (63 g, 462.81 mmol, 1 eq) in THE (500 mL) was added m-CPBA (140.94 g, 694.21 mmol, 85% purity, 1.5 eq). The mixture was stirred at 25° C. for 12 hr. TLC indicated the reaction was completed. The reaction mixture was diluted with petroleum ether (500 mL), and then filtered. The cake was collected and dried under reduced pressure. Compound 4-fluoro-7-oxido-1H-pyrrolo[2,3-b]pyridin-7-ium (123 g, 404.27 mmol, 87.35% yield, 50% purity) was obtained as a white solid. ¹H NMR was recorded. The crude product was used for the next step without further purification.

### Synthesis of methyl 6-chloro-4-fluoro-pyrrolo[2,3-b]pyridine-1-carboxylate

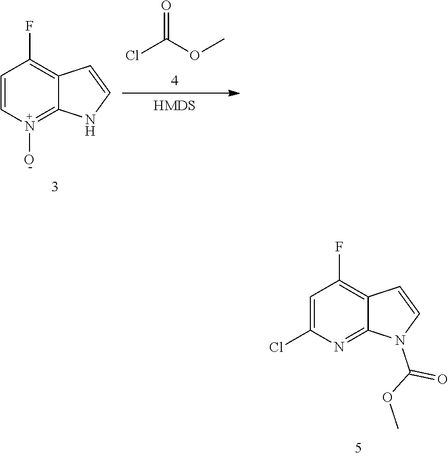

To a solution of 4-fluoro-7-oxido-1H-pyrrolo[2,3-b]pyridin-7-ium (50 g, 164.34 mmol, 50% purity, 1 eq) in THF (500 mL) was added HMDS (26.52 g, 164.34 mmol, 34.45 mL, 1 eq), followed by methyl carbonochloridate (46.59 g, 493.01 mmol, 38.19 mL, 3 eq) (47.340 g). The mixture was stirred at 50° C. for 1 hr. LC-MS showed desired compound methyl 6-chloro-4-fluoro-pyrrolo[2,3-b]pyridine-1-carboxylate. LCMS (ESI) m/z 229.0 [M+H]⁺. The reaction mixture was used directly for the next step without any work up.

### Synthesis of 6-chloro-4-fluoro-1H-pyrrolo[2,3-b]pyridine

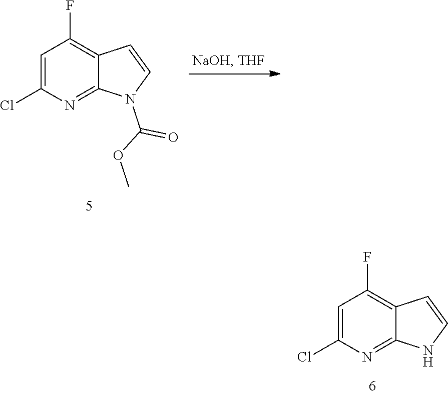

To the reaction mixture from previous step was added NaOH (3 M, 250 mL, 4.63 eq). The mixture was stirred at 25° C. for 2 hr. LC-MS showed desired compound was detected. The reaction mixture was concentrated under reduced pressure to remove THF. The residue was diluted with H₂O (500 mL) and extracted with EtOAc (300 mL×3). The combined organic layer was dried over Na₂SO₄, filtered and concentrated under reduced pressure. The resulting residue was purified by column chromatography (SiO₂, petroleum ether/ethyl acetate=1/0 to 5/1) to afford 6-chloro-4-fluoro-1H-pyrrolo[2,3-b]pyridine (14 g, 73.87 mmol, 45.64% yield, 90% purity) as a white solid.

LCMS (ESI) m/z 171.0 [M+H]⁺; ¹H NMR was recorded.

### Synthesis of (6-chloro-4-fluoro-pyrrolo[2,3-b]pyridin-1-yl)-triisopropyl-silane

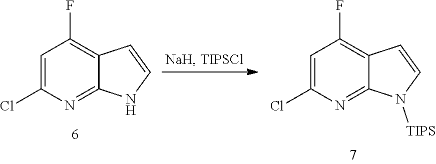

To a solution of 6-chloro-4-fluoro-1H-pyrrolo[2,3-b]pyridine (31 g, 181.74 mmol, 1 eq) in THE (300 mL) was added NaH (21.81 g, 545.23 mmol, 60% purity, 3 eq) at 0° C. under N₂, followed by TIPSCl (42.05 g, 218.09 mmol, 46.67 mL, 1.2 eq) dropwise at 0° C. The reaction mixture was stirred at 0° C. for 2 hr. TLC indicated one major new spot with lower polarity. The reaction mixture was pure into saturated NH₄Cl (300 mL), extracted with EtOAc (200 mL×3). The combined organic layer was dried over Na₂SO₄, filtered and concentrated under reduced pressure. The resulting residue was purified by column chromatography (SiO₂, petroleum ether/ethyl acetate=1/0 to 10/1) to afford (6-chloro-4-fluoro-pyrrolo[2,3-b]pyridin-1-yl)-triisopropyl-silane (59 g, 153.41 mmol, 84.41% yield, 85% purity) as a yellow oil. ¹H NMR was recorded.

### Synthesis of (5,6-dichloro-4-fluoro-pyrrolo[2,3-b]pyridin-1-yl)-triisopropyl-silane

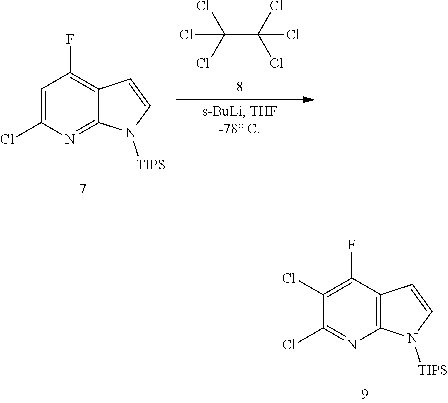

To a solution of (6-chloro-4-fluoro-pyrrolo[2,3-b]pyridin-1-yl)-triisopropyl-silane (10 g, 30.59 mmol, 1 eq) in THE (100 mL) was added a solution of s-BuLi (1.3 M in n-hexane, 44.71 mL, 1.9 eq). The mixture was stirred at −78° C. for 0.5 hr. Then a solution of 1,1,1,2,2,2-hexachloroethane (10.86 g, 45.88 mmol, 5.20 mL, 1.5 eq) in THF (20 mL) was added. The reaction mixture was stirred at −78° C. for 1.5 hr. LC-MS showed desired compound was detected. The reaction mixture was quenched with saturated NH₄Cl (100 mL) at −78° C., and then extracted with EtOAc mL (200 mL×3). The combined organic layer was dried over Na₂SO₄, filtered and concentrated under reduced pressure. The resulting residue was purified by column chromatography (SiO₂, petroleum ether/ethyl acetate=1/0 to 20/1) to afford (5,6-dichloro-4-fluoro-pyrrolo[2,3-b]pyridin-1-yl)-triisopropyl-silane (10 g, 24.91 mmol, 81.42% yield, 90% purity) as a colorless oil. ¹H NMR was recorded.

### Synthesis of 5,6-dichloro-4-fluoro-1H-pyrrolo[2,3-b]pyridine

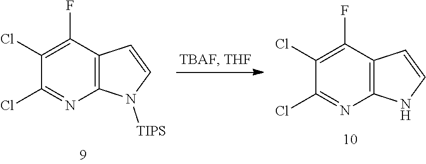

To a solution of (5,6-dichloro-4-fluoro-pyrrolo[2,3-b]pyridin-1-yl)-triisopropyl-silane (10 g, 17.99 mmol, 65% purity, 1 eq) in THF (100 mL) was added TBAF (1 M in THF, 26.98 mL, 1.5 eq). The mixture was stirred at 25° C. for 30 min. TLC indicated the reaction was completed. The reaction mixture was concentrated under reduced pressure to remove THF. The resulting residue was triturated with water (100 mL) for 20 min, filtered and the cake was washed with petroleum ether (20 mL×3). The cake was collected and dried under reduced pressure. Compound 5,6-dichloro-4-fluoro-1H-pyrrolo[2,3-b]pyridine (3.35 g, 14.71 mmol, 81.76% yield, 90% purity) was obtained as a yellow solid, which was used for the next step without further purification. ¹H NMR was recorded.

### Synthesis of 5,6-dichloro-4-fluoro-1-(p-tolylsulfonyl)pyrrolo[2,3-b]pyridine

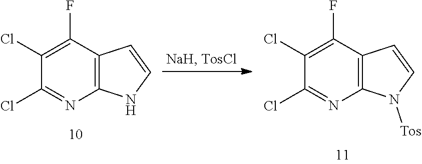

To a mixture of 5,6-dichloro-4-fluoro-1H-pyrrolo[2,3-b]pyridine (3.35 g, 16.34 mmol, 1 eq) in THF (50 mL) was added NaH (1.96 g, 49.02 mmol, 60% purity, 3 eq) at 0° C. under N₂, followed by TosCl (4.67 g, 24.51 mmol, 1.5 eq) in THF (30 mL) dropwise at 0° C. The reaction mixture was stirred at 0° C. for 0.5 hr. LC-MS showed desired compound was detected. The reaction mixture was pure into saturated NH₄Cl (150 mL), and then extracted with EtOAc (100 mL×3).

The combined organic layer was dried over Na₂SO₄, filtered and concentrated under reduced pressure. The resulting residue was purified by column chromatography (SiO₂, petroleum ether/ethyl acetate=1/0 to 10/1) to afford 5,6-dichloro-4-fluoro-1-(p-tolylsulfonyl)pyrrolo[2,3-b]pyridine (2.88 g, 7.22 mmol, 44.16% yield, 90% purity) as a white solid.

LCMS (ESI) m/z 359.0 [M+H]⁺; ¹H NMR was recorded.

### Synthesis of methyl 5,6-dichloro-4-fluoro-1-(p-tolylsulfonyl)pyrrolo[2,3-b]pyridine-2-carboxylate

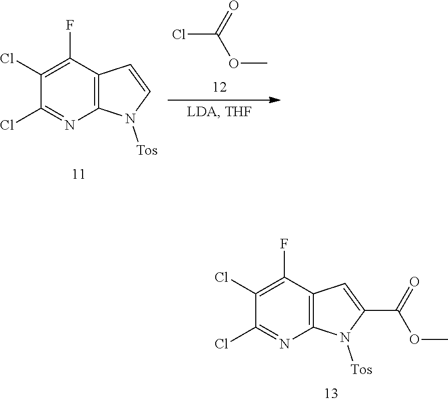

The reaction was conducted at 8.02 mmol (compound 11 from previous step) using the same procedures described in Example 151 (compound 1 to compound 2). The crude product was purified by column chromatography (SiO₂, petroleum ether/ethyl acetate=1/0 to 10/1) to afford methyl 5,6-dichloro-4-fluoro-1-(p-tolylsulfonyl)pyrrolo[2,3-b]pyridine-2-carboxylate (2.3 g, 4.96 mmol, 61.88% yield, 90% purity) as a white solid.

LCMS (ESI) m/z 419.0 [M+H]⁺; ¹H NMR was recorded.

### Synthesis of methyl 5-chloro-4-fluoro-6-methyl-1-(p-tolylsulfonyl)pyrrolo[2,3-b]pyridine-2-carboxylate

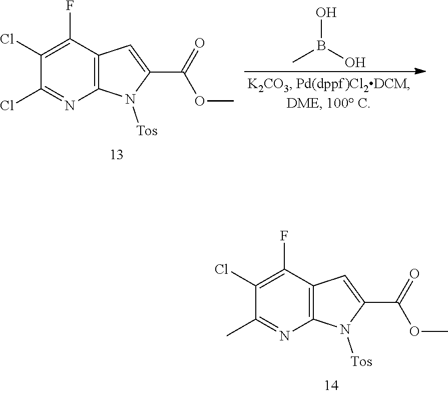

The reaction was conducted at 5.51 mmol (compound 13 from previous step) using the same procedures described in Example 151 (compound 2 to compound 3). The crude product was purified by column chromatography (SiO₂, petroleum ether/ethyl acetate=1/0 to 10/1) to afford methyl 5-chloro-4-fluoro-6-methyl-1-(p-tolylsulfonyl)pyrrolo[2,3-b]pyridine-2-carboxylate (436 mg, 988.86 umol, 17.94% yield, 90% purity) as a white solid.

LCMS (ESI) m/z 397.1 [M+H]⁺; ¹H NMR was recorded.

### Synthesis of methyl 5-chloro-4-fluoro-6-methyl-1H-pyrrolo[2,3-b]pyridine-2-carboxylate

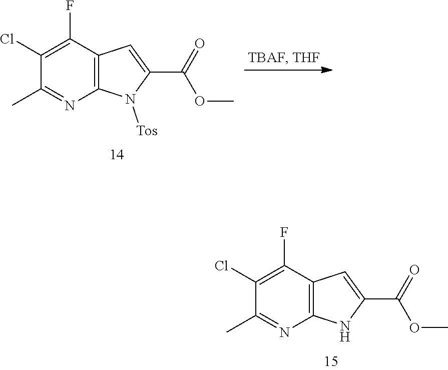

To a solution of methyl 5-chloro-4-fluoro-6-methyl-1-(p-tolylsulfonyl)pyrrolo[2,3-b]pyridine-2-carboxylate (436 mg, 1.10 mmol, 1 eq) in THE (5 mL) was added TBAF (1 M HCl, 2 mL, 1.82 eq). The mixture was stirred at 25° C. for 0.5 hr. TLC indicated reactant 14 was consumed completely. The reaction mixture was concentrated under reduced pressure to remove THF. The resulting residue was triturated with water (20 mL) for 20 min, filtered. The cake was collected, washed with petroleum ether (10 mL×3), and concentrated under reduced pressure. Compound methyl 5-chloro-4-fluoro-6-methyl-1H-pyrrolo[2,3-b]pyridine-2-carboxylate (260 mg, 964.42 umol, 87.78% yield, 90% purity) was obtained as a white solid. ¹H NMR was recorded. The crude product was used for the next step without purification.

### Synthesis of 5-chloro-4-fluoro-6-methyl-1H-pyrrolo[2,3-b]pyridine-2-carboxylic acid

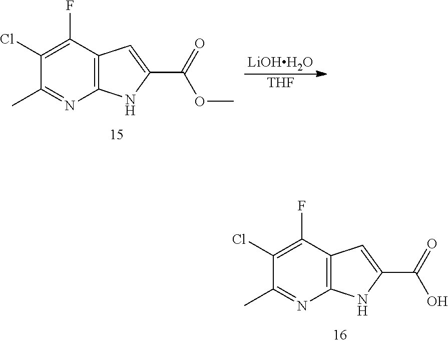

To a solution of methyl 5-chloro-4-fluoro-6-methyl-1H-pyrrolo[2,3-b]pyridine-2-carboxylate (260 mg, 1.07 mmol, 1 eq) in THE (5 mL) and H₂O (5 mL) was added LiOH.H₂O (224.82 mg, 5.36 mmol, 5 eq). The mixture was stirred at 25° C. for 12 hr. LC-MS showed desired product was detected. The reaction mixture was concentrated under reduced pressure to remove THF. The residue was diluted with water (10 mL), adjusted to pH to 2 with aqueous HCl (6M), then filtered. The filtrate was concentrated in vacuo to afford crude compound 5-chloro-4-fluoro-6-methyl-1H-pyrrolo[2,3-b]pyridine-2-carboxylic acid (230 mg, 905.48 umol, 84.50% yield, 90% purity) as a white solid.

LCMS (ESI) m/z 229.0 [M+H]⁺; ¹H NMR was recorded.

### Synthesis of 5-chloro-N-(1,1-dimethylsilepan-4-yl)-4-fluoro-6-methyl-1H-pyrrolo[2,3-b]pyridine-2-carboxamide, 5-chloro-N-[(4R)-1,1-dimethylsilepan-4-yl]-4-fluoro-6-methyl-1H-pyrrolo[2,3-b]pyridine-2-carboxamide and 5-chloro-N-[(4S)-1,1-dimethylsilepan-4-yl]-4-fluoro-6-methyl-1H-pyrrolo[2,3-b]pyridine-2-carboxamide

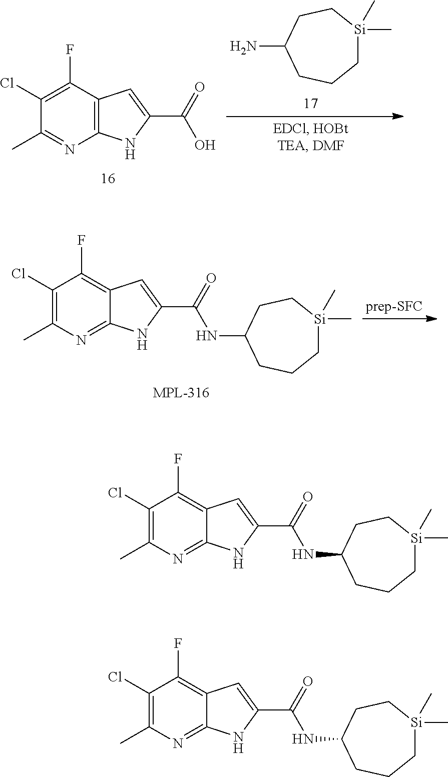

To a solution of 5-chloro-4-fluoro-6-methyl-1H-pyrrolo[2,3-b]pyridine-2-carboxylic acid (150 mg, 656.15 umol, 1 eq) and 1,1-dimethylsilepan-4-amine (152.59 mg, 787.38 umol, 1.2 eq, HCl salt) in DMF (3 mL) at 25° C. was added a solution of HOBt (265.98 mg, 1.97 mmol, 3 eq) and EDCI (377.35 mg, 1.97 mmol, 3 eq) in DMF (2 mL), followed by TEA (331.98 mg, 3.28 mmol, 456.64 uL, 5 eq). The reaction mixture was stirred 25° C. for 2 hr. LC-MS showed desired compound was detected. The mixture was purified by prep-HPLC (column: Phenomenex Synergi C18 150*30 mm*4 um; mobile phase: A: 0.225% formic acid in water; B: CH₃CN; gradient: 65%-95% B over 11 min) to afford MPL-316, which was further purified by SFC (Berger MG II, column: DAICEL CHIRALPAK AD (250 mm*30 mm, 10 um); mobile phase: A: 0.1% NH₃H₂O in EtOH, B: CO₂; isocratic 45% B, flow rate: 80 min/mL) to afford two peaks (two enantiomers), 5-chloro-N-[(4R)-1,1-dimethylsilepan-4-yl]-4-fluoro-6-methyl-1H-pyrrolo[2,3-b]pyridine-2-carboxamide and 5-chloro-N-[(4S)-1,1-dimethylsilepan-4-yl]-4-fluoro-6-methyl-1H-pyrrolo[2,3-b]pyridine-2-carboxamide.

Peak 1 (MPL-316A): 45.8 mg, 124.48 umol, 18.97% yield, 100% purity, white solid.

LCMS (ESI) m/z 368.2 [M+H]⁺; ¹H NMR (500 MHz, DMSO-d6) δ=12.47 (br s, 1H), 8.34 (d, J=7.9 Hz, 1H), 7.23 (s, 1H), 3.97-3.79 (m, 1H), 2.62 (s, 3H), 2.02-1.39 (m, 6H), 0.85-0.53 (m, 4H), 0.04 (d, J=8.9 Hz, 6H).

Peak 2 (MPL-316B): 51.1 mg, 138.89 umol, 21.17% yield, 100% purity, white solid.

LCMS (ESI) m/z 368.2 [M+H]⁺; ¹H NMR (500 MHz, DMSO-d6) δ=12.47 (br s, 1H), 8.34 (d, J=7.9 Hz, 1H), 7.23 (s, 1H), 4.04-3.75 (m, 1H), 2.62 (s, 3H), 2.05-1.33 (m, 6H), 0.87-0.53 (m, 4H), 0.04 (d, J=8.9 Hz, 6H).

MPL-316A and MPL-316B were also analyzed by analytical SFC.

### Conditions:

Instrument: CAS-SH-ANA-SFC-K (Waters UPCC with PDA Detector)
Column: Chiralpak AD-3 50*4.6 mm, 3 um particle size
Mobile phase: A: CO₂, B: 0.05% DEA in ethanol

### Isocratic: 40% B

Flow rate: 2.5 mL/min
Column temp.: 35° C.

### ABPR: 1500 psi

MPL-316A: retention time: 3.47 min; 100% ee; MPL-316B: retention time: 3.85 min; 100% ee.

### Example 153. MPL-387

### Synthesis of N-(1,1-dimethylsilinan-4-yl)-6-methoxy-1H-pyrrolo[2,3-b]pyridine-2-carboxamid

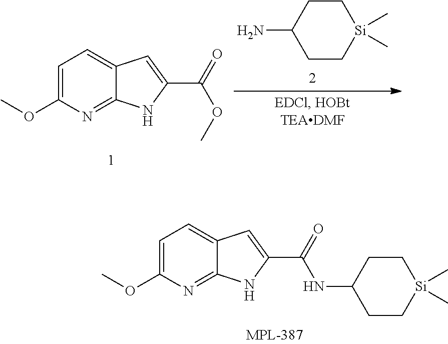

To a solution of 6-methoxy-1H-pyrrolo[2,3-b]pyridine-2-carboxylic acid (50 mg, 260.18 umol, 1 eq) and 1,1-dimethylsilinan-4-amine (56.13 mg, 312.22 umol, 1.2 eq, HCl salt) in DMF (2 mL) was added a solution of EDCI (149.63 mg, 780.55 umol, 3 eq) and HOBt (105.47 mg, 780.55 umol, 3 eq) in DMF (1 mL), followed by TEA (131.64 mg, 1.30 mmol, 181.07 uL, 5 eq). The mixture was stirred at 25° C. for 1 hr. LC-MS showed desired compound was detected. The reaction mixture was filtered. The filtrated was purified by prep-HPLC (column: Phenomenex Synergi C18 150*30 mm*4 um; mobile phase: A: 0.225% formic acid in water; B: CH3CN; gradient: 49%-79% B over 11 min). Compound N-(1,1-dimethylsilinan-4-yl)-6-methoxy-1H-pyrrolo[2,3-b]pyridine-2-carboxamide (62.4 mg, 195.11 umol, 74.99% yield, 99.260% purity) was obtained as a white solid.

LCMS m/z: 318.1 [M+1]⁺; ¹H NMR (500 MHz, DMSO-d6) 3=11.99-11.70 (m, 1H), 8.03-7.88 (m, 2H), 7.01 (d, J=1.8 Hz, 1H), 6.57 (d, J=8.5 Hz, 1H), 3.93-3.82 (m, 3H), 3.75-3.64 (m, 1H), 2.03-1.92 (m, 2H), 1.65-1.49 (m, 2H), 0.85-0.71 (m, 2H), 0.59 (dt, J=4.7, 13.9 Hz, 2H), 0.14-−0.04 (m, 6H).

### Example 154. MPL-388

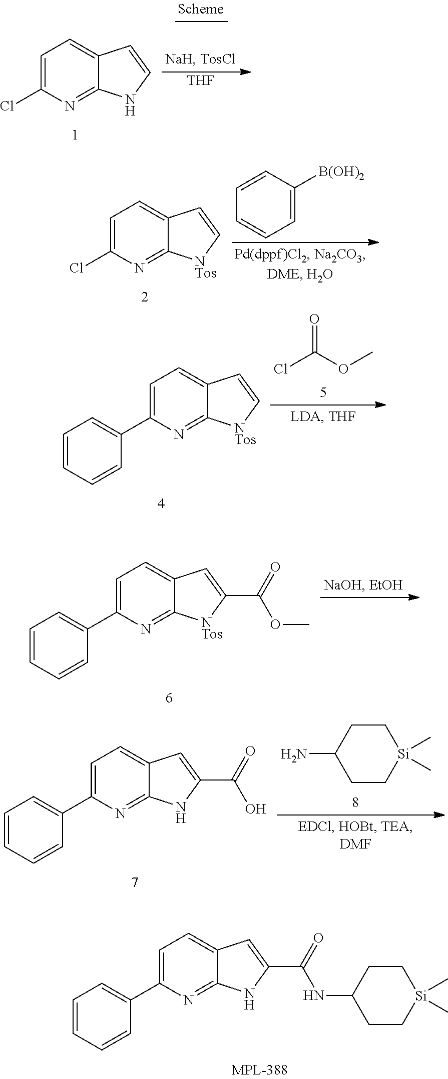

### Synthesis of 6-chloro-1-(p-tolylsulfonyl)pyrrolo[2,3-b]pyridine

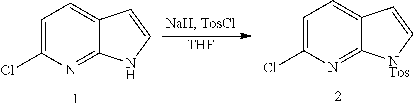

To a cooled solution of 6-chloro-1H-pyrrolo[2,3-b]pyridine (2 g, 13.11 mmol, 1 eq) in THE (20 mL) was added NaH (786.39 mg, 19.66 mmol, 60% purity, 1.5 eq) in batches and stirred at 0° C. for 30 min. Then to the mixture was added TosCl (3.00 g, 15.73 mmol, 1.2 eq) in batches. The mixture was stirred at 0° C. for 30 min. LC-MS showed desired compound was detected. The reaction mixture was quenched with saturated NH₄Cl solution (20 mL) at 25° C., and then diluted with water (10 mL) and extracted with EtOAc (20 mL×2). The combined organic layer was washed with brine (20 mL×2), dried over Na₂SO₄, filtered and concentrated under reduced pressure. The resulting residue was purified by column chromatography (SiO₂, petroleum ether/ethyl acetate=1/0 to 5/1). Compound 6-chloro-1-(p-tolylsulfonyl)pyrrolo[2,3-b]pyridine (2.84 g, 8.34 mmol, 63.66% yield, 90% purity) was obtained as a white solid. ¹H NMR was recorded.

### Synthesis of 6-phenyl-1-(p-tolylsulfonyl)pyrrolo[2,3-b]pyridine

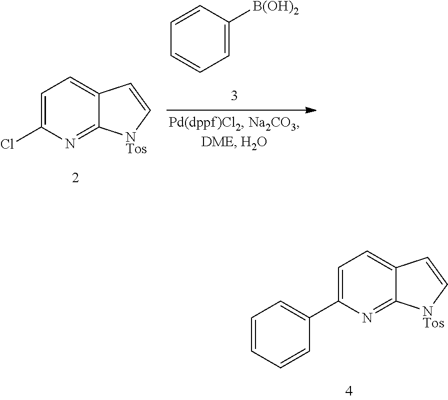

To a mixture of 6-chloro-1-(p-tolylsulfonyl)pyrrolo[2,3-b]pyridine (1.3 g, 4.24 mmol, 1 eq), phenylboronic acid (775.06 mg, 6.36 mmol, 1.5 eq) and K₂CO₃ (1.17 g, 8.48 mmol, 2 eq) was added dioxane (15 mL). The mixture was purged with N₂ and Pd(dppf)Cl₂.CH₂Cl₂ (346.07 mg, 423.77 umol, 0.1 eq) was added under N₂. The mixture was stirred at 110° C. for 12 hr. LC-MS showed desired compound was detected. The mixture was filtered, the cake was washed with EtOAc (50 mL×2), the combined filtrate was dried over Na₂SO₄ and concentrated in vacuo. The residue was purified by column chromatography (SiO2, Petroleum ether/Ethyl acetate=1/0 to 3/1) to afford 6-phenyl-1-(p-tolylsulfonyl)pyrrolo[2,3-b]pyridine (1.7 g, 4.39 mmol, 90.11% yield, 90% purity) as a brown solid.

LCMS (ESI) m/z 349.1 [M+H]⁺; ¹H NMR was recorded.

### Synthesis of methyl 6-phenyl-1-(p-tolylsulfonyl)pyrrolo[2,3-b]pyridine-2-carboxylate

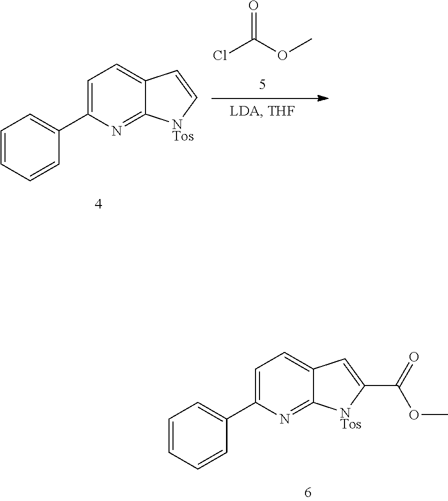

A mixture of 6-phenyl-1-(p-tolylsulfonyl)pyrrolo[2,3-b]pyridine (498.24 mg, 1.43 mmol, 1 eq) in THE (5 mL) was degassed and purged with N₂ for 3 times, and LDA (2 M in THF, 1.07 mL, 1.5 eq) was then added and stirred at −60° C. for 10 min under N₂ atmosphere. Then methyl carbonochloridate (676.09 mg, 7.15 mmol, 554.17 uL, 5 eq) was added and stirred at −60° C. for 30 min. LC-MS showed desired compound was detected. The reaction mixture was quenched with saturated NH₄Cl (20 mL) at 25° C., and then diluted with water (20 mL) and extracted with EtOAc (20 mL×2). The combined organic layer was washed with brine (20 mL×2), dried over Na₂SO₄, filtered and concentrated under reduced pressure. The resulting residue was purified by column chromatography (SiO₂, petroleum ether/ethyl acetate=1/0 to 3/1) to afford methyl 6-phenyl-1-(p-tolylsulfonyl)pyrrolo[2,3-b]pyridine-2-carboxylate (463 mg, 1.03 mmol, 71.69% yield, 90% purity) as a brown solid.

LCMS (ESI) m/z 407.1 [M+H]⁺; ¹H NMR was recorded.

### Synthesis of 6-phenyl-1H-pyrrolo[2,3-b]pyridine-2-carboxylic acid

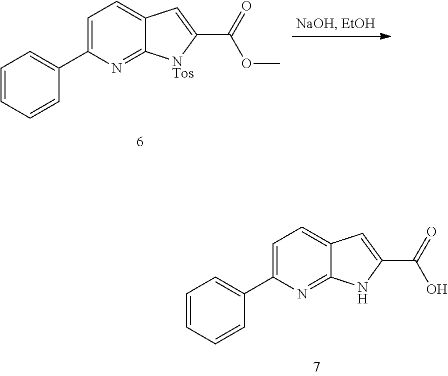

To a solution of methyl 6-phenyl-1-(p-tolylsulfonyl)pyrrolo[2,3-b]pyridine-2-carboxylate (463 mg, 1.14 mmol, 1 eq) in EtOH (3 mL) was added NaOH (2 M in water, 3 mL, 5.27 eq). The mixture was stirred at 80° C. for 12 hr. TLC showed one major new spot with higher polarity. The reaction mixture was concentrated under reduced pressure to remove EtOH, and then treated with aqueous HCl (6 M) until pH turned to 2, filtered and concentrated under reduced pressure to afford crude 6-phenyl-1H-pyrrolo[2,3-b]pyridine-2-carboxylic acid (200 mg, 755.54 umol, 66.33% yield, 90% purity) as a yellow solid. It was used for the next step without further purification.

¹H NMR (400 MHz, DMSO-d6) δ=13.12 (br s, 1H), 12.35 (s, 1H), 8.19-8.11 (m, 3H), 7.75 (d, J=8.2 Hz, 1H), 7.57-7.39 (m, 4H), 7.13 (d, J=2.0 Hz, 1H).

### Synthesis of N-(1,1-dimethylsilinan-4-yl)-6-phenyl-1H-pyrrolo[2,3-b]pyridine-2-carboxamide

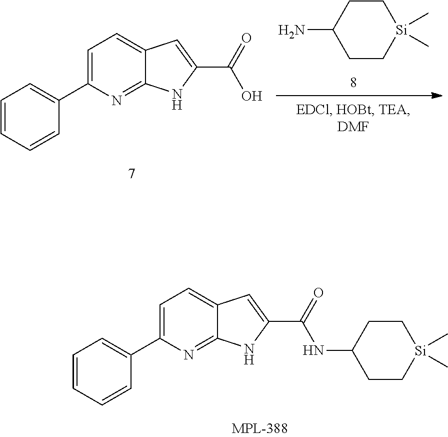

To a solution of 6-phenyl-1H-pyrrolo[2,3-b]pyridine-2-carboxylic acid (50 mg, 209.87 umol, 1 eq) and 1,1-dimethylsilinan-4-amine (41.50 mg, 230.86 umol, 1.1 eq, HCl salt) in DMF (1 mL). was added a solution of HOBt (85.08 mg, 629.61 umol, 3 eq) and EDCI (120.70 mg, 629.61 umol, 3 eq) in DMF (1 mL), followed by TEA (106.18 mg, 1.05 mmol, 146.06 uL, 5 eq). The reaction mixture was stirred at 25° C. for 2 hr. LC-MS showed desired compound was detected. The mixture was purified by prep-HPLC (column: YMC-Actus Triart C18 150*30 mm*5 um; mobile phase: A: 0.225% formic acid in water; B: CH3CN; gradient: 64%-94% B over 11 min) to afford N-(1,1-dimethylsilinan-4-yl)-6-phenyl-1H-pyrrolo[2,3-b]pyridine-2-carboxamide (42.9 mg, 115.65 umol, 55.11% yield, 98% purity) as a yellow solid.

LCMS (ESI) m/z 364.1 [M+H]⁺; ¹H NMR (500 MHz, DMSO-d6) δ=12.07 (s, 1H), 8.21 (d, J=8.1 Hz, 1H), 8.16-8.07 (m, 3H), 7.71 (d, J=8.4 Hz, 1H), 7.53-7.46 (m, 2H), 7.44-7.37 (m, 1H), 7.12 (d, J=2.0 Hz, 1H), 3.78-3.66 (m, 1H), 2.05-1.92 (m, 2H), 1.67-1.51 (m, 2H), 0.87-0.54 (m, 4H), 0.15-0.01 (m, 6H).

### Example 155. MPL-389

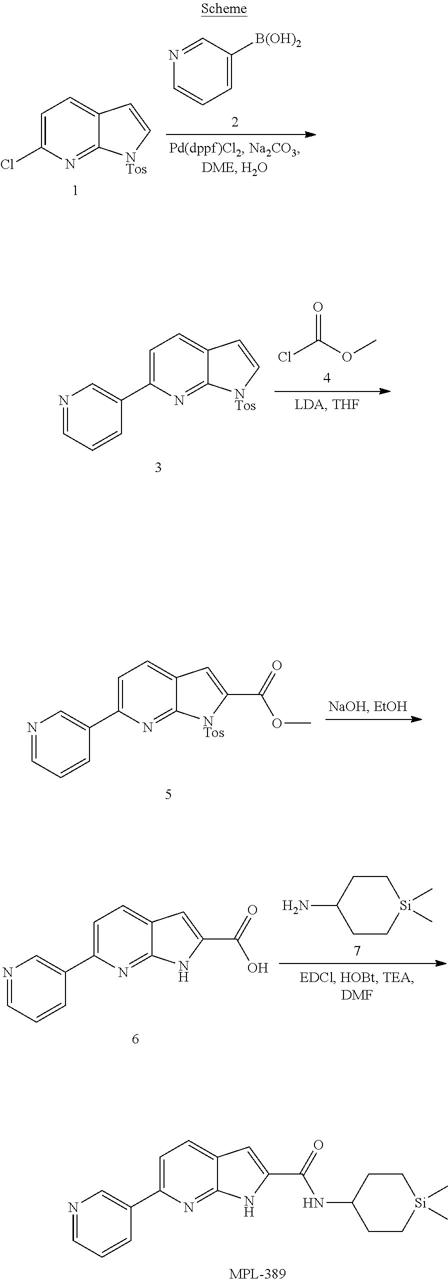

### Synthesis of 1-(p-tolylsulfonyl)-6-(3-pyridyl)pyrrolo[2,3-b]pyridine

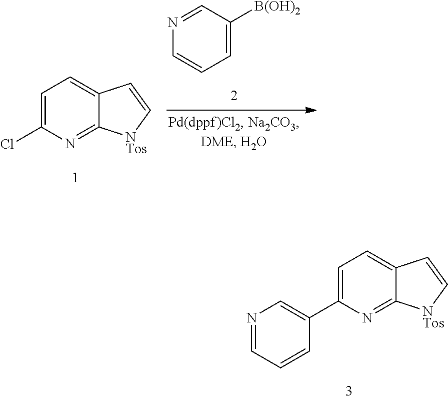

To a mixture of 6-chloro-1-(p-tolylsulfonyl)pyrrolo[2,3-b]pyridine (1.3 g, 4.24 mmol, 1 eq), 3-pyridylboronic acid (781.34 mg, 6.36 mmol, 1.5 eq) and K₂CO₃ (1.17 g, 8.48 mmol, 2 eq) was added dioxane (15 mL) and H₂O (0.1 mL). The mixture was purged with N₂ and Pd(dppf)Cl₂.CH₂Cl₂ (346.07 mg, 423.77 umol, 0.1 eq) was added under N₂. The mixture was stirred at 110° C. for 12 hr. LC-MS showed desired compound was detected. The mixture was filtered. The cake was washed with EtOAc (50 mL×2). The combined filtrate was dried over Na₂SO₄ and concentrated in vacuo. The residue was purified by column chromatography (SiO₂, Petroleum ether/Ethyl acetate=1/0 to 2/1). Compound 1-(p-tolylsulfonyl)-6-(3-pyridyl)pyrrolo[2,3-b]pyridine (967 mg, 2.49 mmol, 58.78% yield, 90% purity) was obtained as a yellow solid.

LCMS (ESI) m/z 350.1 [M+H]⁺; ¹H NMR was recorded.

### Synthesis of methyl 1-(p-tolylsulfonyl)-6-(3-pyridyl)pyrrolo[2,3-b]pyridine-2-carboxylate

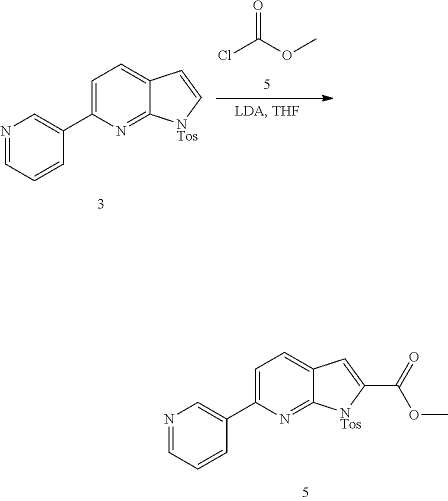

A mixture of 1-(p-tolylsulfonyl)-6-(3-pyridyl)pyrrolo[2,3-b]pyridine (498.56 mg, 1.43 mmol, 1 eq) in THE (5 mL) was degassed and purged with N₂ for 3 times. LDA (2 M in THF, 1.07 mL, 1.5 eq) was added and stirred at −60° C. for 10 min under N₂ atmosphere. Methyl carbonochloridate (674.18 mg, 7.13 mmol, 552.61 uL, 5 eq) was then added and stirred at −60° C. for 30 min. LC-MS showed desired compound was detected. The reaction mixture was quenched with saturated NH₄Cl solution (20 mL) at 25° C., and then diluted with water (20 mL) and extracted with EtOAc (20 mL×2). The combined organic layer was washed with brine (20 mL×2), dried over Na₂SO₄, filtered and concentrated under reduced pressure. The resulting residue was purified by column chromatography (SiO₂, dichloromethane/methanol=1/0 to 10/1) to afford methyl 1-(p-tolylsulfonyl)-6-(3-pyridyl)pyrrolo[2,3-b]pyridine-2-carboxylate (480 mg, 589.04 umol, 41.28% yield, 50% purity) as a yellow solid.

LCMS (ESI) m/z 408.1 [M+H]⁺; ¹H NMR was recorded.

### Synthesis of 6-(3-pyridyl)-1H-pyrrolo[2,3-b]pyridine-2-carboxylic acid

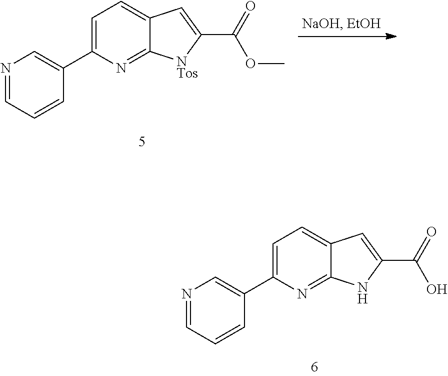

To a solution of methyl 1-(p-tolylsulfonyl)-6-(3-pyridyl)pyrrolo[2,3-b]pyridine-2-carboxylate (480 mg, 1.18 mmol, 1 eq) in EtOH (3 mL) was added aqueous NaOH (2 M, 3 mL, 5.09 eq). The reaction mixture was stirred at 80° C. for 2 hr. LC-MS showed desired compound was detected. The reaction mixture was concentrated under reduced pressure to remove EtOH. The aqueous phase was treated with aqueous HCl (6 M) until pH turned to 6, filtered and concentrated under reduced pressure to afford 6-(3-pyridyl)-1H-pyrrolo[2,3-b]pyridine-2-carboxylic acid (crude, 240 mg, 802.58 umol, 68.13% yield, 80% purity) as a yellow solid.

LCMS (ESI) m/z 240.1 [M+H]⁺; ¹H NMR (400 MHz, DMSO-d6) δ=12.33 (br s, 1H), 9.31 (d, J=1.6 Hz, 1H), 8.62 (dd, J=1.6, 4.7 Hz, 1H), 8.46 (br d, J=8.2 Hz, 1H), 8.20 (d, J=8.6 Hz, 1H), 7.81 (d, J=8.6 Hz, 1H), 7.53 (dd, J=4.7, 7.4 Hz, 1H), 7.14-7.03 (m, 1H).

### Synthesis of N-(1,1-dimethylsilinan-4-yl)-6-(3-pyridyl)-1H-pyrrolo[2,3-b]pyridine-2-carboxamide

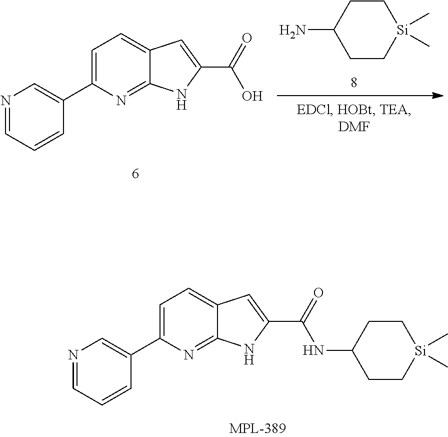

To a solution of 6-(3-pyridyl)-1H-pyrrolo[2,3-b]pyridine-2-carboxylic acid (50 mg, 209.00 umol, 1 eq) and 1,1-dimethylsilinan-4-amine (41.33 mg, 229.91 umol, 1.1 eq, HCl salt) in DMF (1 mL) was added a solution of HOBt (84.72 mg, 627.01 umol, 3 eq) and EDCI (120.20 mg, 627.01 umol, 3 eq) in DMF (1 mL), followed by TEA (105.74 mg, 1.05 mmol, 145.45 uL, 5 eq). The reaction mixture was stirred at 25° C. for 2 hr. LC-MS showed desired compound was detected. The mixture was purified by prep-HPLC (column: YMC-Actus Triart C18 150*30 mm*5 um; mobile phase: A: 0.225% formic acid in water; B: CH3CN; gradient: 45%-75% B over 11 min) to afford N-(1,1-dimethylsilinan-4-yl)-6-(3-pyridyl)-1H-pyrrolo[2,3-b]pyridine-2-carboxamide (31.5 mg, 86.42 umol, 41.35% yield, 100% purity) as a brown solid.

LCMS (ESI) m/z 365.0 [M+H]⁺; ¹H NMR (500 MHz, DMSO-d6) δ=12.18 (s, 1H), 9.31 (d, J=2.1 Hz, 1H), 8.61 (dd, J=1.5, 4.7 Hz, 1H), 8.46 (td, J=1.9, 8.0 Hz, 1H), 8.25 (d, J=7.9 Hz, 1H), 8.19 (d, J=8.2 Hz, 1H), 7.80 (d, J=8.4 Hz, 1H), 7.59-7.46 (m, 1H), 7.15 (d, J=2.0 Hz, 1H), 3.81-3.66 (m, 1H), 2.06-1.91 (m, 2H), 1.68-1.51 (m, 2H), 0.79 (br d, J=14.6 Hz, 2H), 0.62 (dt, J=4.7, 14.0 Hz, 2H), 0.15-0.04 (m, 6H).

### Example 156. MPL-390

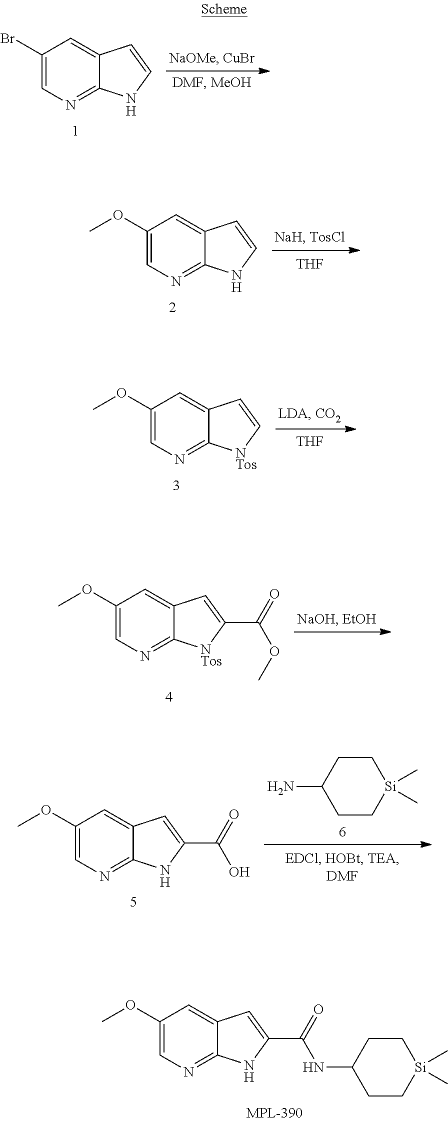

### Synthesis of 5-methoxy-1H-pyrrolo[2,3-b]pyridine

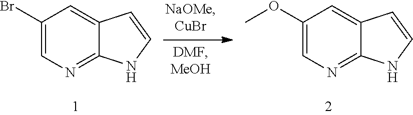

To a solution of 5-bromo-1H-pyrrolo[2,3-b]pyridine (1.24 g, 6.29 mmol, 1 eq) in DMF (40 mL) and MeOH (30 mL) was added CuBr (1.81 g, 12.59 mmol, 383.35 uL, 2 eq) and sodium methanolate (18.02 g, 333.55 mmol, 53 eq). The mixture was stirred at 130° C. for 4 hr under N₂ atmosphere. LC-MS showed desired product was detected. The reaction mixture was filtered and concentrated under reduced pressure to remove solvent. The residue was diluted with H₂O (200 mL) and extracted with EtOAc (50 mL×3). The combined organic layer was washed with brine (30 mL×2), dried over Na₂SO₄, filtered and concentrated under reduced pressure. The resulting residue was purified by column chromatography (SiO₂, dichloromethane/methanol=1/0 to 10/1). Compound 5-methoxy-1H-pyrrolo[2,3-b]pyridine (641 mg, 3.68 mmol, 50.37% yield, 85% purity) was obtained as a brown solid.

LCMS m/z: 149.1 [M+1]⁺; ¹H NMR was recorded.

### Synthesis of 5-methoxy-1-(p-tolylsulfonyl)pyrrolo[2,3-b]pyridine

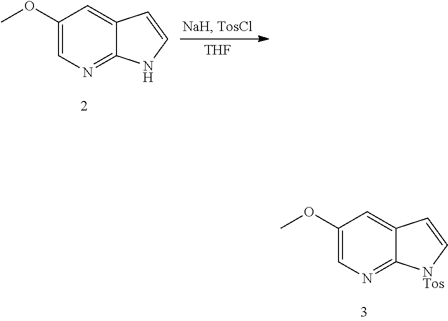

To a solution of 5-methoxy-1H-pyrrolo[2,3-b]pyridine (641 mg, 4.33 mmol, 1 eq) in THE (8 mL) was added NaH (259.56 mg, 6.49 mmol, 60% purity, 1.5 eq). The mixture was stirred at 0° C. for 30 mins. TosCl (907.29 mg, 4.76 mmol, 1.1 eq) was then added. The mixture was stirred at 0° C. for 30 mins. LC-MS showed desired mass was detected. The reaction was quenched with saturated NH₄Cl (30 mL) and extracted with EtOAc (20 mL×2). The combined organic layer was washed with brine (20 mL×2), dried over Na₂SO₄, filtered and concentrated under reduced pressure. The resulting residue was purified by column chromatography (SiO₂, petroleum ether/ethyl acetate=1/0 to 5/1) to afford 5-methoxy-1-(p-tolylsulfonyl)pyrrolo[2,3-b]pyridine (1.10 g, 3.45 mmol, 79.82% yield, 95% purity) as a white solid.

LCMS m/z: 303.1 [M+1]⁺; ¹H NMR was recorded.

### Synthesis of methyl 5-methoxy-1-(p-tolylsulfonyl)pyrrolo[2,3-b]pyridine-2-carboxylate

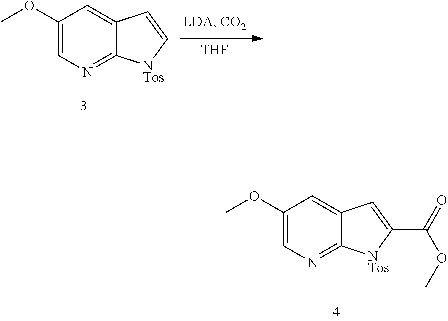

To a solution of 5-methoxy-1-(p-tolylsulfonyl)pyrrolo[2,3-b]pyridine (1.10 g, 3.64 mmol, 1 eq) in THE (10 mL) was added LDA in THE (2 M, 2.73 mL, 1.5 eq) dropwise at −78° C. under N₂. The reaction mixture was stirred at −78° C. for 30 min. Methyl carbonochloridate (1.72 g, 18.19 mmol, 1.41 mL, 5 eq) was added dropwise at −78° C. The reaction mixture was stirred at −78° C. for another 30 mins. TLC (Petroleum ether:Ethyl acetate=3:1) indicated new spots formed. The reaction mixture was quenched with saturated NH₄Cl (20 mL), extracted with EtOAc (30 mL×2). The combined organic layer was washed with brine (20 mL), dried over Na₂SO₄, filtered and concentrated under reduced pressure. The resulting residue was purified by column chromatography (SiO₂, Petroleum ether/Ethyl acetate=1/0 to 10/1) to afford methyl 5-methoxy-1-(p-tolylsulfonyl)pyrrolo[2,3-b]pyridine-2-carboxylate (514 mg, 1.35 mmol, 37.24% yield, 95% purity) as a white solid. ¹H NMR was recorded.

### Synthesis of 5-methoxy-1H-pyrrolo[2,3-b]pyridine-2-carboxylic acid

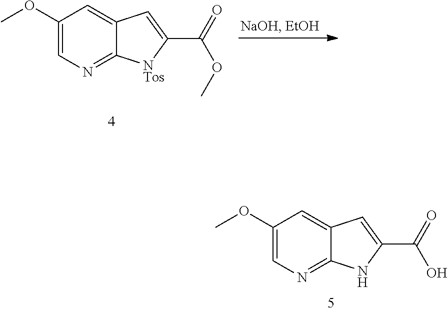

To a solution of methyl 5-methoxy-1-(p-tolylsulfonyl)pyrrolo[2,3-b]pyridine-2-carboxylate (514 mg, 1.43 mmol, 1 eq) in EtOH (6 mL) was added aqueous NaOH (2 M, 6 mL, 8.41 eq). The mixture was stirred at 80° C. for 2 hr. LC-MS showed desired mass. The reaction mixture was concentrated under reduced pressure to remove EtOH. The aqueous solution was adjusted to pH to 3-4 with aqueous HCl (6 N) and filtered. The cake was washed with petroleum ether (15 mL) and dried under reduced pressure. Compound 5-methoxy-1H-pyrrolo[2,3-b]pyridine-2-carboxylic acid (245 mg, 1.21 mmol, 84.92% yield, 95% purity) was obtained as a white solid

LCMS m/z: 193.1 [M+1]⁺; ¹H NMR (400 MHz, DMSO-d6) δ=13.40-12.69 (m, 1H), 12.29-12.08 (m, 1H), 8.14 (d, J=2.9 Hz, 1H), 7.61 (d, J=2.7 Hz, 1H), 7.01 (d, J=2.1 Hz, 1H), 3.82 (s, 3H).

### Synthesis of N-(1, 1-dimethylsilinan-4-yl)-5-methoxy-1H-pyrrolo[2,3-b]pyridine-2-carboxamide

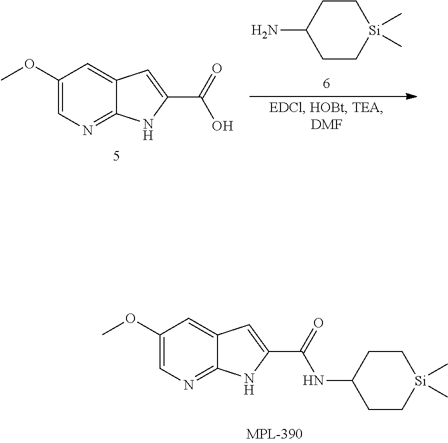

To a solution of 5-methoxy-1H-pyrrolo[2,3-b]pyridine-2-carboxylic acid (50 mg, 260.18 umol, 1 eq) and 1,1-dimethylsilinan-4-amine (56.13 mg, 312.22 umol, 1.2 eq, HCl salt) in DMF (2 mL) was added a solution of EDCI (149.63 mg, 780.55 umol, 3 eq) and HOBt (105.47 mg, 780.55 umol, 3 eq) in DMF (0.5 mL), followed by TEA (157.97 mg, 1.56 mmol, 217.29 uL, 6 eq). The mixture was stirred at 25° C. for 1 hr. LC-MS showed desired mass. The reaction mixture was filtered to obtain filtrate. The residue was purified by prep-HPLC (column: YMC-Actus Triart C18 150*30 mm*5 um; mobile phase: A: 0.225% formic acid in water; B: CH₃CN; gradient:52%-82% B over 11 min) to afford N-(1, 1-dimethylsilinan-4-yl)-5-methoxy-1H-pyrrolo[2,3-b]pyridine-2-carboxamide (57.4 mg, 180.74 umol, 69.47% yield, 99.960% purity) as a white solid. LCMS m/z: 318.0 [M+1]⁺; ¹H NMR (500 MHz, DMSO-d₆) δ=11.88 (br s, 1H), 8.17 (d, J=8.1 Hz, 1H), 8.06 (d, J=2.9 Hz, 1H), 7.59 (d, J=2.7 Hz, 1H), 7.08-6.91 (m, 1H), 3.84-3.78 (m, 3H), 3.75-3.66 (m, 1H), 2.05-1.93 (m, 2H), 1.66-1.52 (m, 2H), 0.78 (br d, J=14.5 Hz, 2H), 0.61 (dt, J=4.8, 14.1 Hz, 2H), 0.11-0.01 (m, 6H).

### Example 157. MPL-391

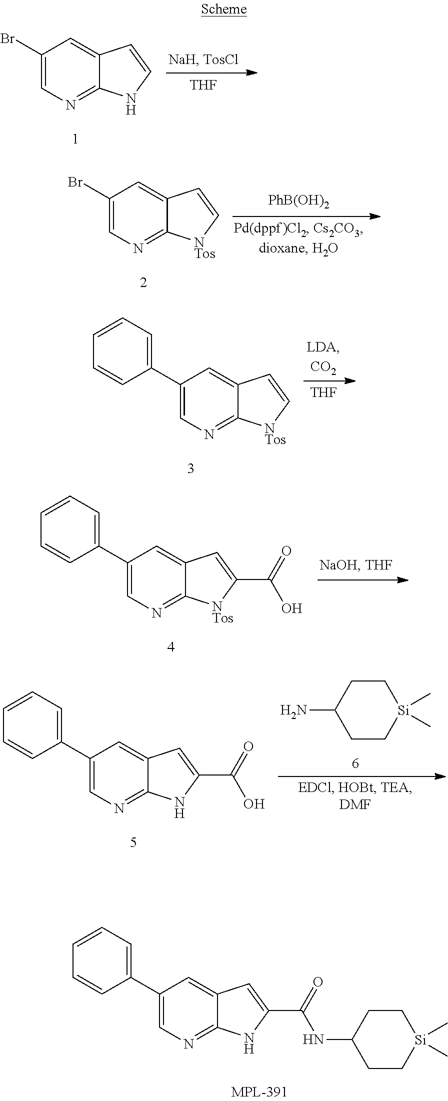

### Synthesis of 5-bromo-1-(p-tolylsulfonyl)pyrrolo[2,3-b]pyridine

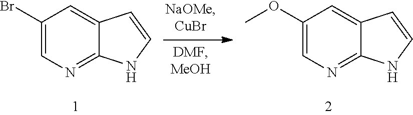

To a solution of 5-bromo-1H-pyrrolo[2,3-b]pyridine (2 g, 10.15 mmol, 1 eq) in THE (20 mL) was added NaH (608.98 mg, 15.23 mmol, 60% purity, 1.5 eq). The mixture was stirred at 0° C. for 30 mins. Then TosCl (2.13 g, 11.17 mmol, 1.1 eq) was added. The mixture was stirred at 0° C. for 30 mins. TLC (Petroleum ether:Ethyl acetate=10:1) indicated many new spots formed. The reaction was quenched with saturated NH₄Cl (30 mL) and extracted with EtOAc (20 mL×2). The combined organic layer was washed with brine (20 mL×2), dried over Na₂SO₄, filtered and concentrated under reduced pressure. The residue was purified by column chromatography (SiO₂, Petroleum ether/Ethyl acetate=1/0 to 10/1) to afford 5-bromo-1-(p-tolylsulfonyl)pyrrolo[2,3-b]pyridine (3.01 g, 7.72 mmol, 76.01% yield, 90% purity) as a white solid. ¹H NMR was recorded.

### Synthesis of 5-phenyl-1-(p-tolylsulfonyl)pyrrolo[2,3-b]pyridine

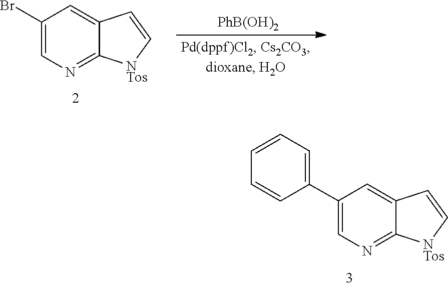

To a mixture of 5-bromo-1-(p-tolylsulfonyl)pyrrolo[2,3-b]pyridine (1.5 g, 4.27 mmol, 1 eq), phenylboronic acid (624.89 mg, 5.13 mmol, 1.2 eq) and Cs₂CO₃ (2.78 g, 8.54 mmol, 2 eq) in dioxane (0.5 mL) and H₂O (5 mL) was added Pd(dppf)Cl₂ (312.50 mg, 427.09 umol, 0.1 eq) under N₂. The mixture was heated at 110° C. for 12 hrs. LC-MS indicated desired product was detected. The mixture was diluted with EtOAc (30 mL) and filtered to remove insoluble materials. The filtrate was concentrated in vacuo. The residue was purified by column chromatography (SiO₂, Petroleum ether/Ethyl acetate=1/0 to 25/3). Compound 5-phenyl-1-(p-tolylsulfonyl)pyrrolo[2,3-b]pyridine (1.36 g, 3.32 mmol, 77.69% yield, 85% purity) was obtained as a colorless oil.

LCMS m/z: 349.1 [M+1]⁺; ¹H NMR was recorded.

### Synthesis of methyl 5-phenyl-1-(p-tolylsulfonyl)pyrrolo[2,3-b]pyridine-2-carboxylate

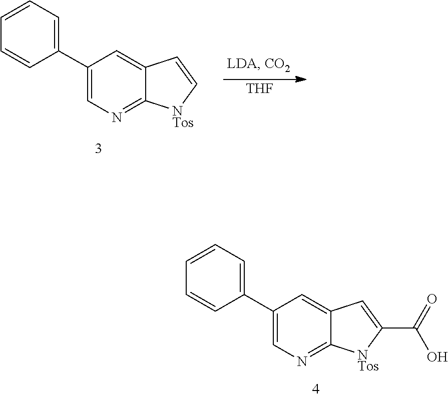

To a solution of 5-phenyl-1-(p-tolylsulfonyl)pyrrolo[2,3-b]pyridine (1.36 g, 3.90 mmol, 1 eq) in THE (12 mL) was added LDA (2 M in THF, 2.93 mL, 1.5 eq) dropwise at −78° C. under N₂. After stirring at −78° C. for 30 min, methyl carbonochloridate (1.84 g, 19.52 mmol, 1.51 mL, 5 eq) was added dropwise at −78° C., and the reaction mixture was stirred at −78° C. for another 30 min. TLC (Petroleum ether:Ethyl acetate=5:1) indicated new spots were formed. The reaction mixture was quenched with NH₄Cl (20 mL) and extracted with EtOAc (30 mL×2). The combined organic layer was washed with brine (20 mL), dried over Na₂SO₄, filtered and concentrated under reduced pressure. The resulting residue was purified by column chromatography (SiO₂, Petroleum ether/Ethyl acetate=1/0 to 5/1). Compound methyl 5-phenyl-1-(p-tolylsulfonyl)pyrrolo[2,3-b]pyridine-2-carboxylate (317 mg, 701.93 umol, 17.98% yield, 90% purity) was obtained as a white solid. ¹H NMR was recorded.

### Synthesis of 5-phenyl-1H-pyrrolo[2,3-b]pyridine-2-carboxylic acid

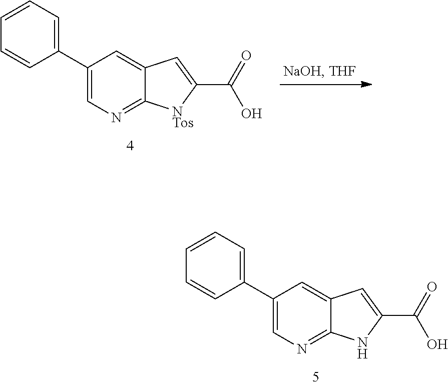

To a solution of methyl 5-phenyl-1-(p-tolylsulfonyl)pyrrolo[2,3-b]pyridine-2-carboxylate (317 mg, 779.92 umol, 1 eq) in EtOH (5 mL) was added aqueous NaOH (2 M, 5 mL, 12.82 eq). The mixture was stirred at 80° C. for 2 hr. Desired product was detected by LC-MS. The reaction mixture was concentrated under reduced pressure to remove EtOH. The residual solution was adjusted to pH 3-4 with aqueous HCl (6 N), and then filtered. The cake was washed with petroleum ether (15 mL) and dried under reduced pressure. Compound 5-phenyl-1H-pyrrolo[2,3-b]pyridine-2-carboxylic acid (163 mg, 615.76 umol, 78.95% yield, 90% purity) was obtained as a yellow solid. It was used for the next step without further purification.

LCMS m/z: 239.1 [M+1]⁺; ¹H NMR (500 MHz, DMSO-d6) δ=12.43 (s, 1H), 8.73 (, 1H), 8.35-8.39 (s, 1H), 7.53-7.76 (d, J=7.5 Hz, 2H), 7.48-7.53 (t, J=7.5 Hz, 2H), 7.37-7.40 (m, 1H), 7.16 (s, 1H).

### Synthesis of N-(1,1-dimethylsilinan-4-yl)-5-phenyl-1H-pyrrolo[2,3-b]pyridine-2-carboxamide

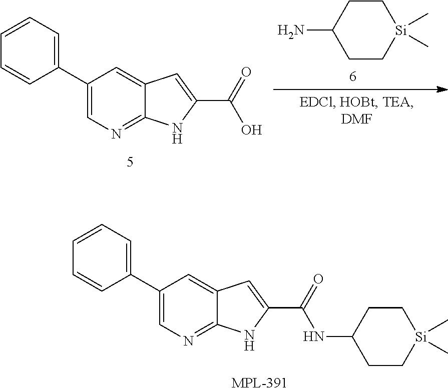

To a solution of 5-phenyl-1H-pyrrolo[2,3-b]pyridine-2-carboxylic acid (50 mg, 209.87 umol, 1 eq) and 1,1-dimethylsilinan-4-amine (45.27 mg, 251.85 umol, 1.2 eq, HCl salt) in DMF (1.5 mL) was added a solution of EDCI (120.70 mg, 629.61 umol, 3 eq) and HOBt (85.08 mg, 629.61 umol, 3 eq) in DMF (1 mL), followed by TEA (127.42 mg, 1.26 mmol, 175.27 uL, 6 eq). The mixture was stirred at 25° C. for 1 hr. LC-MS indicated desired product was formed. The reaction mixture was filtered. The filtrate was purified by prep-HPLC (column: YMC-Actus Triart C18 150*30 mm*5 um; mobile phase: A: 0.225% formic acid in water; B: CH3CN; gradient: 66%-95% over 11 min) to afford N-(1,1-dimethylsilinan-4-yl)-5-phenyl-1H-pyrrolo[2,3-b]pyridine-2-carboxamide (48.8 mg, 133.42 umol, 63.57% yield, 99.39% purity) as a white solid.

LCMS m/z: 239.1 [M+1]⁺; ¹H NMR (500 MHz, DMSO-d₆) δ=12.12 (s, 1H), 8.62 (d, J=2.1 Hz, 1H), 8.36-8.21 (m, 2H), 7.73 (d, J=7.3 Hz, 2H), 7.49 (t, J=7.7 Hz, 2H), 7.42-7.30 (m, 1H), 7.22-7.09 (m, 1H), 3.78-3.67 (m, 1H), 2.06-1.95 (m, 2H), 1.68-1.54 (m, 2H), 0.79 (br d, J=14.6 Hz, 2H), 0.62 (dt, J=4.7, 14.0 Hz, 2H), 0.14-0.02 (m, 6H).

### Example 158. MPL-392

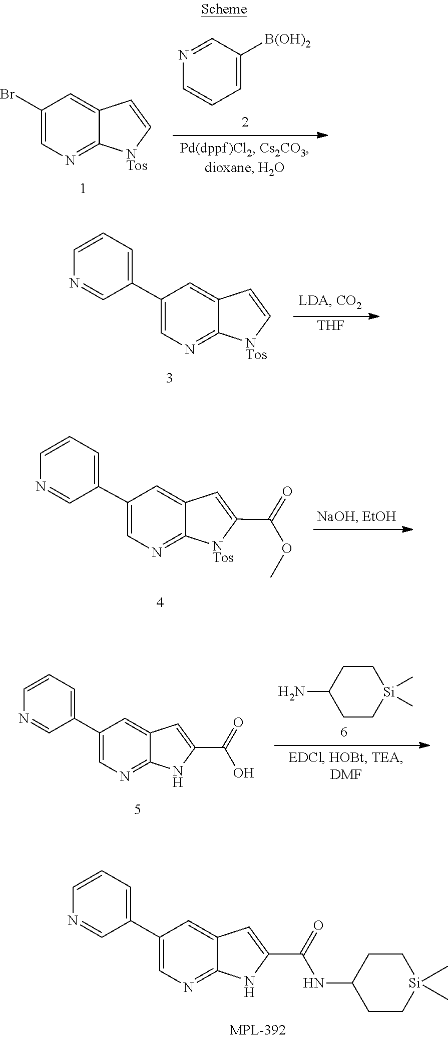

### Synthesis of 1-(p-tolylsulfonyl)-5-(3-pyridyl)pyrrolo[2,3-b]pyridine

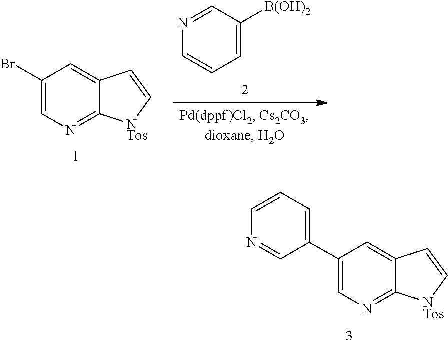

To a solution of 5-bromo-1-(p-tolylsulfonyl)pyrrolo[2,3-b]pyridine (2.18 g, 6.21 mmol, 1 eq), 3-pyridylboronic acid (915.53 mg, 7.45 mmol, 1.2 eq) and Cs₂CO₃ (4.04 g, 12.41 mmol, 2 eq) in dioxane (20 mL) and H2O (2 mL) was added Pd(dppf)Cl₂ (454.17 mg, 620.70 umol, 0.1 eq) under N₂. The mixture was heated at 110° C. for 12 hrs. LC-MS showed desired product was detected. The mixture was diluted with EtOAc (30 mL) and filtered to remove the insoluble solid. The filtrate was concentrated in vacuo. The residue was purified by column chromatography (SiO₂, Petroleum ether/Ethyl acetate=1/0 to 5/3). Compound 1-(p-tolylsulfonyl)-5-(3-pyridyl)pyrrolo[2,3-b]pyridine (1.86 g, 4.80 mmol, 77.35% yield, 90% purity) was obtained as a yellow solid.

LCMS m/z: 350.1 [M+1]⁺; ¹H NMR was recorded.

### Synthesis of methyl 1-(p-tolylsulfonyl)-5-(3-pyridyl)pyrrolo[2,3-b]pyridine-2-carboxylate

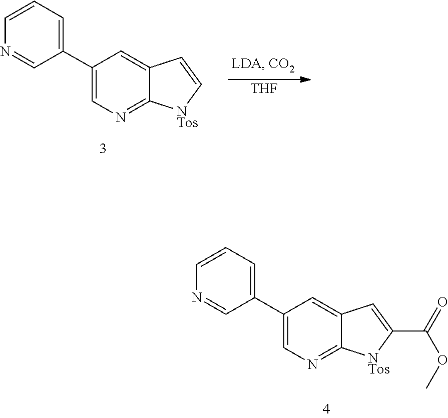

To a solution of 1-(p-tolylsulfonyl)-5-(3-pyridyl)pyrrolo[2,3-b]pyridine (1.86 g, 5.33 mmol, 1 eq) in THE (20 mL) was added LDA (2 M in THF, 4.00 mL, 1.5 eq) dropwise at −78° C. under N₂. The reaction mixture was stirred at −78° C. for 30 min. Methyl carbonochloridate (2.52 g, 26.67 mmol, 2.07 mL, 5 eq) was added dropwise at −78° C. The reaction mixture was stirred at −78° C. for another 30 min. TLC (Petroleum ether:Ethyl acetate=1:1) indicated compound 3 was remained and new spots formed. The reaction mixture was quenched with saturated NH₄Cl (50 mL) and extracted with dichloromethane (30 mL×3). The combined organic layer was washed with brine (30 mL), dried over Na₂SO₄, filtered and concentrated under reduced pressure. The resulting residue was purified by column chromatography (SiO₂, Petroleum ether/Ethyl acetate=1/0 to 0/1). Compound methyl 1-(p-tolylsulfonyl)-5-(3-pyridyl) pyrrolo[2,3-b]pyridine-2-carboxylate (1.36 g, 2.34 mmol, 43.80% yield, 70% purity) was obtained as a white solid. ¹H NMR was recorded. It was used for the next step without further purification.

### Synthesis of 5-(3-pyridyl)-1H-pyrrolo[2,3-b]pyridine-2-carboxylic acid

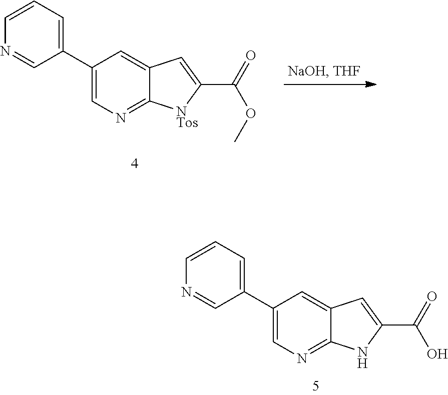

To a solution of methyl 1-(p-tolylsulfonyl)-5-(3-pyridyl)pyrrolo[2,3-b]pyridine-2-carboxylate (700 mg, 1.72 mmol, 1 eq) in EtOH (10 mL) was added aqueous NaOH (2 M, 859.02 uL, 1 eq). The mixture was stirred at 80° C. for 2 hr. The reaction mixture was concentrated under reduced pressure. The residue was diluted with H₂O (8 mL) and extracted with dichloromethane (10 mL×3). The aqueous phase was adjusted to pH 6-7 with aqueous HCl (6 N) and filtered. The filter cake was washed with petroleum ether (10 mL) and dried under reduced pressure. Compound 5-(3-pyridyl)-1H-pyrrolo[2,3-b]pyridine-2-carboxylic acid (270 mg, 1.07 mmol, 62.41% yield, 95% purity) was obtained as a white solid. It was used for the next step without further purification.

LCMS m/z: 240.1 [M+1]⁺; ¹H NMR (500 MHz, DMSO-d₆) δ=13.25 (br, s, 1H), 12.51 (s, 1H), 8.97 (s, 1H), 8.75 (s, 1H), 8.59 (s, 1H), 8.44 (s, 1H), 8.16-8.18 (d, J=7.5 Hz, 1H), 7.51-7.54 (m, 1H), 7.17 (s, 1H).

### Synthesis of N-(1,1-dimethylsilinan-4-yl)-5-(3-pyridyl)-1H-pyrrolo[2,3-b]pyridine-2-carboxamide

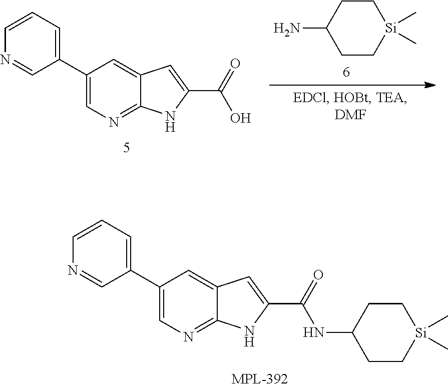

To a solution of 5-(3-pyridyl)-1H-pyrrolo[2,3-b]pyridine-2-carboxylic acid (50 mg, 209.00 umol, 1 eq) and 1,1-dimethylsilinan-4-amine (45.09 mg, 250.81 umol, 1.2 eq, HCl salt) in DMF (1.5 mL) was added a solution of EDCI (40.07 mg, 209.00 umol, 1 eq) and HOBt (28.24 mg, 209.00 umol, 1 eq) in DMF (1 mL), followed by TEA (21.15 mg, 209.00 umol, 29.09 uL, 1 eq). The mixture was stirred at 25° C. for 1 hr. LC-MS indicated desired product was detected. The reaction mixture was filtered. The filtrate was purified by prep-HPLC (column: YMC-Actus Triart C18 150*30 mm*5 um; mobile phase: A: 0.225% formic acid in water; B: CH3CN; gradient: 42%-72% B over 11 min). Compound N-(1,1-dimethylsilinan-4-yl)-5-(3-pyridyl)-1H-pyrrolo[2,3-b]pyridine-2-carboxamide (41 mg, 111.67 umol, 53.43% yield, 99.279% purity) was obtained as a white solid.

LCMS m/z: 365.1 [M+1]⁺; ¹H NMR (500 MHz, DMSO-d₆) δ=12.21 (s, 1H), 8.97 (d, J=2.0 Hz, 1H), 8.99-8.93 (m, 1H), 8.68 (d, J=2.1 Hz, 1H), 8.58 (d, J=3.8 Hz, 1H), 8.42 (d, J=2.1 Hz, 1H), 8.32 (d, J=8.1 Hz, 1H), 8.16 (br d, J=7.9 Hz, 1H), 7.51 (dd, J=4.7, 7.9 Hz, 1H), 7.23-7.16 (m, 1H), 3.79-3.67 (m, 1H), 2.08-1.95 (m, 2H), 1.68-1.54 (m, 2H), 0.79 (br d, J=14.5 Hz, 2H), 0.62 (dt, J=4.7, 14.0 Hz, 2H), 0.10 (s, 3H), 0.04 (s, 3H).

### Example 159: MPL-401, MPL-401A and MPL-401B

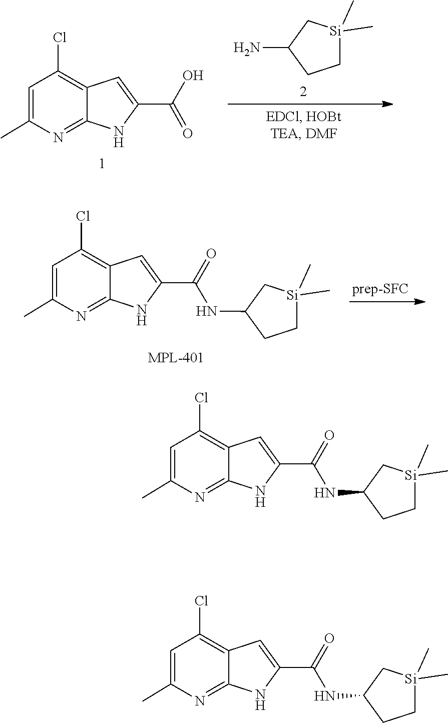

### Synthesis of 4-chloro-N-(1,1-dimethylsilolan-3-yl)-6-methyl-1H-pyrrolo[2,3-b]pyridine-2-carboxamide, 4-chloro-N-[(3R)-1,1-dimethylsilolan-3-yl]-6-methyl-1H-pyrrolo[2,3-b]pyridine-2-carboxamide, and 4-chloro-N-[(3S)-1,1-dimethylsilolan-3-yl]-6-methyl-1H-pyrrolo[2,3-b]pyridine-2-carboxamide

To a solution of 4-chloro-6-methyl-1H-pyrrolo[2,3-b]pyridine-2-carboxylic acid (30 mg, 142.44 umol, 1 eq) and 1,1-dimethylsilolan-3-amine (25.97 mg, 156.68 umol, 1.1 eq, HCl salt) in DMF (1 mL) was added a solution of EDCI (54.61 mg, 284.88 umol, 2 eq) and HOBt (38.49 mg, 284.88 umol, 2 eq) in DMF (1 mL), followed by TEA (57.65 mg, 569.76 umol, 79.30 uL, 4 eq). The mixture was stirred at 20° C. for 2 hr. LC-MS showed reactant 1 was consumed completely and one main peak with desired mass. The mixture was diluted with MeOH (2 mL) and filtered to remove insoluble matter. The filtrate was purified by prep-HPLC (column: YMC-Actus Triart C18 150*30 mm*5 um; mobile phase: A: 0.225% formic acid in water; B: CH₃CN; gradient: 57%-87% B over 11 min). Compound 4-chloro-N-(1,1-dimethylsilolan-3-yl)-6-methyl-1H-pyrrolo[2,3-b]pyridine-2-carboxamide (27.9 mg, 86.68 umol, 60.85% yield, 100% purity) was obtained as a white solid.

LCMS (ESI) m/z 322.0 [M+H]⁺; ¹H NMR (400 MHz, DMSO-d₆) δ=12.08 (s, 1H), 8.21 (br d, J=7.6 Hz, 1H), 7.01 (d, J=2.2 Hz, 1H), 6.98 (s, 1H), 3.92-3.77 (m, 1H), 2.35 (s, 3H), 1.92-1.81 (m, 1H), 1.27 (dq, J=7.2, 12.1 Hz, 1H), 0.98-0.89 (m, 1H), 0.64 (br dd, J=5.5, 14.6 Hz, 1H), 0.48 (dd, J=11.2, 14.2 Hz, 1H), 0.41-0.30 (m, 1H), 0.00 (d, J=1.5 Hz, 6H).

The same reaction was conducted later at 427.3 umol. The product (MPL-401) from prep-HPLC purification was separated by prep-SFC (Waters Prep SFC 80Q; Column: (s,s) WHELK-O1 (250 mm*30 mm, Sum); mobile phase: A: 0.1% NH₃H₂O in IPA, B: CO₂, isocratic 30% B, flow rate: 40 mL/min) to yield two peaks (two enantiomers), 4-chloro-N-[(3R)-1,1-dimethylsilolan-3-yl]-6-methyl-1H-pyrrolo[2,3-b]pyridine-2-carboxamide, and 4-chloro-N-[(3S)-1,1-dimethylsilolan-3-yl]-6-methyl-1H-pyrrolo[2,3-b]pyridine-2-carboxamide.

Peak 1 (MPL-401A) (12.8 mg, 38.47 umol, 35.3% yield, 96.7% purity) was obtained as a white solid.

LCMS m/z: 322.0 [M+1]⁺; ¹H NMR (400 MHz, DMSO-d6) δ=12.24 (br s, 1H), 8.38 (d, J=7.4 Hz, 1H), 7.17 (d, J=11.7 Hz, 2H), 4.09-3.95 (m, 1H), 2.52 (s, 3H), 2.03 (br d, J=4.3 Hz, 1H), 1.44 (dq, J=7.0, 12.0 Hz, 1H), 1.11 (br dd, J=5.1, 14.1 Hz, 1H), 0.81 (br dd, J=5.3, 14.7 Hz, 1H), 0.65 (dd, J=11.2, 14.3 Hz, 1H), 0.58-0.47 (m, 1H), 0.18 (d, J=1.6 Hz, 6H).

Peak 2 (MPL-401B) (42.4 mg, 131.73 umol, 30.3% yield, 100% purity) was obtained as a white solid.

LCMS m/z: 322.0 [M+1]⁺; ¹1H NMR (500 MHz, DMSO-d6) δ=12.24 (br s, 1H), 8.38 (d, J=7.6 Hz, 1H), 7.17 (d, J=15.0 Hz, 2H), 4.02 (dq, J=7.0, 11.6 Hz, 1H), 2.52 (s, 3H), 2.09-1.98 (m, 1H), 1.44 (dq, J=7.2, 12.1 Hz, 1H), 1.16-1.06 (m, 1H), 0.86-0.77 (m, 1H), 0.65 (dd, J=11.1, 14.1 Hz, 1H), 0.53 (ddd, J=7.9, 12.6, 14.6 Hz, 1H), 0.17 (d, J=2.1 Hz, 6H).

### Example 160. MPL-434

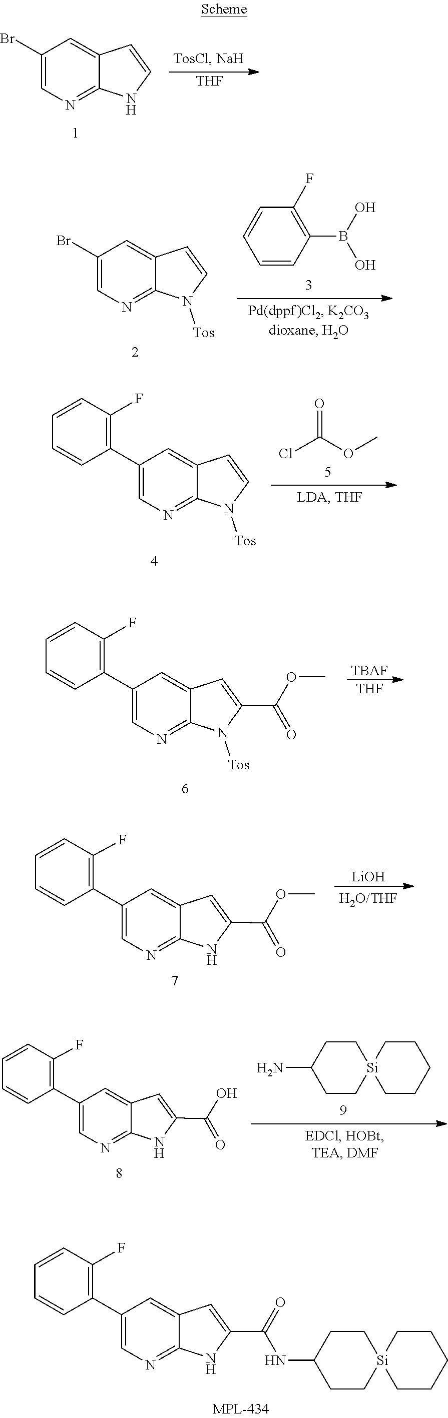

### Synthesis of 5-bromo-1-(p-tolylsulonyl)pyrrolo[2,3-b]pyridine

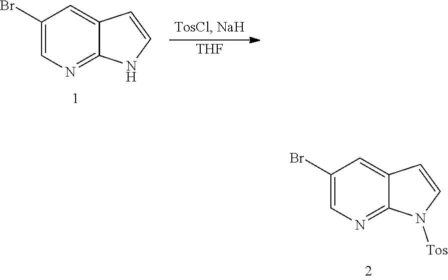

To a solution of 5-bromo-1H-pyrrolo[2,3-b]pyri dine (10 g, 50.75 mmol, 1 eq) in THF (100 mL) was added NaH (6.09 g, 152.26 mmol, 60% purity, 3 eq), followed by TosCl (14.51 g, 76.13 mmol, 1.5 eq) at 0° C. The mixture was stirred at 0° C. for 2 hr. TLC indicated that desired product was detected. The reaction mixture was quenched with aqueous NH₄Cl (100 mL) and extracted with EtOAc (100 mL×3). The combined organic layer was dried over Na₂SO₄, filtered and concentrated under reduced pressure. The resulting residue was purified by column chromatography (SiO₂, Petroleum ether/Ethyl acetate=1/0 to 10/1) to afford 5-bromo-1-(p-tolylsulfonyl)pyrrolo[2,3-b]pyridine (12 g, 30.75 mmol, 60.59% yield, 90% purity) as a yellow solid. ¹H NMR was recorded.

### Synthesis of 5-(2-fluorophenyl)-1-(p-tolylsulfonyl)pyrrolo[2,3-b]pyridine

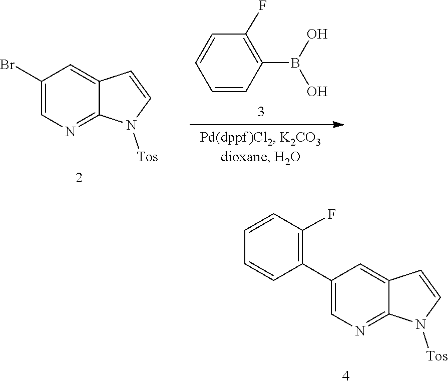

To a mixture of 5-bromo-1-(p-tolylsulfonyl)pyrrolo[2,3-b]pyridine (200 mg, 569.45 umol, 1 eq), (2-fluorophenyl)boronic acid (239.03 mg, 1.71 mmol, 3 eq) and K₂CO₃ (236.10 mg, 1.71 mmol, 3 eq) in dioxane (5 mL) was added H₂O (0.05 mL). The mixture was purged with N₂, Pd(dppf)Cl₂ (41.67 mg, 56.94 umol, 0.1 eq) was then added under N₂. The mixture was stirred at 120° C. for 12 hr under N₂. LCMS showed desired mass. The mixture was filtered. The cake was washed with EtOAc (10 mL×3). The combined filtrate was dried over Na₂SO₄ and concentrated under reduce pressure. The residue was purified by column chromatography (SiO₂, Petroleum ether/Ethyl acetate=1/0 to 1/1). Compound 5-(2-fluorophenyl)-1-(p-tolylsulfonyl)pyrrolo[2,3-b]pyridine (200 mg, 436.67 umol, 76.68% yield, 80% purity) was obtained as a yellow solid.

LCMS (ESI) m/z: 367.1 [M+H]⁺; ¹H NMR was recorded.

### Synthesis of methyl 5-(2-fluorophenyl)-1-(p-tolylsulfonyl)pyrrolo[2,3-b]pyridine-2-carboxylate

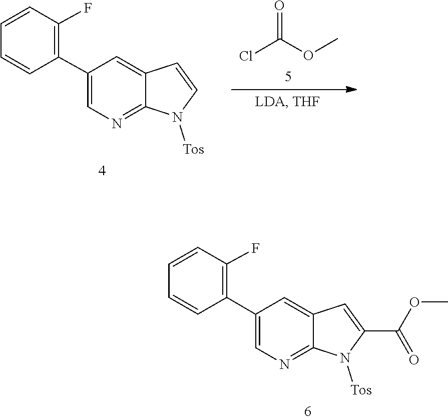

A mixture of 5-(2-fluorophenyl)-1-(p-tolylsulfonyl)pyrrolo[2,3-b]pyridine (2.2 g, 6.00 mmol, 1 eq) in THE (25 mL) was degassed and purged with N₂ for 3 times. LDA (2 M in THF, 4.50 mL, 1.5 eq) was added and the reaction mixture was stirred at −60° C. for 10 min under N₂ atmosphere. Methyl carbonochloridate (2.84 g, 30.02 mmol, 2.33 mL, 5 eq) was then added and the mixture was stirred at −60° C. for 30 min. LC-MS showed that desired compound was detected. The reaction mixture was quenched with saturated NH₄Cl solution 50 mL at 25° C., and then diluted with water (20 mL) and extracted with EtOAc (50 mL×2). The combined organic layer was washed with brine (50 mL×2), dried over Na₂SO₄, filtered and concentrated under reduced pressure. The resulting residue was purified by column chromatography (SiO₂, dichloromethane/methanol=1/0 to 5/1). Compound methyl 5-(2-fluorophenyl)-1-(p-tolylsulfonyl)pyrrolo[2,3-b]pyridine-2-carboxylate (900 mg, 1.70 mmol, 28.25% yield, 80% purity) was obtained as a yellow oil.

LCMS (ESI) m/z: 408.1 [M+H]⁺; ¹H NMR was recorded.

### Synthesis of methyl 5-(2-fluorophenyl)-1H-pyrrolo[2,3-b]pyridine-2-carboxylate

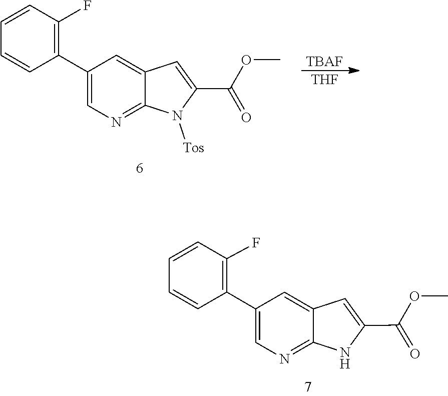

To a solution of methyl 1-(p-tolylsulfonyl)-5-(3-pyridyl)pyrrolo[2,3-b]pyridine-2-carboxylate (800 mg, 1.96 mmol, 1 eq) in THF (10 mL) was added TBAF in THE (1 M, 2.95 mL, 1.5 eq). The mixture was stirred at 25° C. for 12 hr. The reaction mixture was concentrated under reduced pressure to remove solvent. The residue was diluted with H₂O (10 mL) and filtered to obtain compound methyl 5-(3-pyridyl)-1H-pyrrolo[2,3-b]pyridine-2-carboxylate (500 mg, crude) as a yellow solid. It was used for the next step without purification.

### Synthesis of 5-(2-fluorophenyl)-1H-pyrrolo[2,3-b]pyridine-2-carboxylic acid

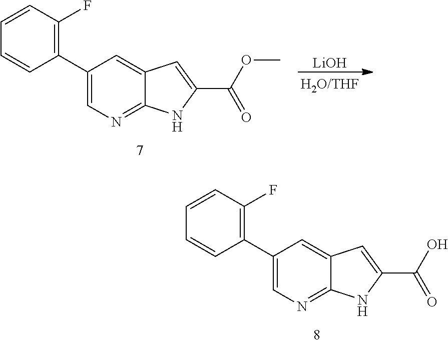

To a solution of methyl 5-(2-fluorophenyl)-1H-pyrrolo[2,3-b]pyridine-2-carboxylate (400 mg, crude, 1.48 mmol, 1 eq) in EtOH (10 mL) was added a solution of LiOH.H₂O (1.24 g, 29.60 mmol, 20 eq) in H₂O (5 mL), the mixture was stirred at 80° C. for 1 hr. TLC showed that reactant was consumed, and new spot was formed. The mixture was concentrated under reduced pressure to remove EtOH. The residue was diluted with water (10 mL), acidified to pH 7 with 1 N HCl, extracted with EtOAc (20 mL×2). The combined organic layer was dried over Na₂SO₄ and concentrated under reduced pressure to afford compound 5-(2-fluorophenyl)-1H-pyrrolo[2,3-b]pyridine-2-carboxylic acid (300 mg, 936.65 umol, 63.28% yield, 80% purity) as a yellow solid. The crude product was used for the next step without further purification.

¹H NMR (500 MHz, DMSO-d6) δ=12.50 (s, 1H), 8.55 (s, 1H), 8.27 (s, 1H), 7.62 (br t, J=7.9 Hz, 1H), 7.51-7.44 (m, 1H), 7.39-7.32 (m, 2H), 7.18 (d, J=1.8 Hz, 1H).

### Synthesis of 5-(2-fluorophenyl)-N-(6-silaspiro[5.5]undecan-3-yl)-1H-pyrrolo[2,3-b]pyridine-2-carboxamide

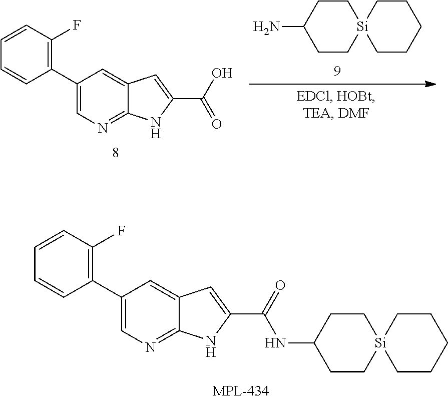 [SMILES: O=C(O)c1cc2cc(-c3ccccc3F)cnc2[nH]1 ; NC1CC[Si]2(CCCCC2)CC1 ; O=C(NC1CC[Si]2(CCCCC2)CC1)c1cc2cc(-c3ccccc3F)cnc2[nH]1]

To a solution of 5-(2-fluorophenyl)-1H-pyrrolo[2,3-b]pyridine-2-carboxylic acid (50 mg, 195.14 umol, 1 eq) and 6-silaspiro[5.5]undecan-3-amine (35.78 mg, 162.77 umol, 1 eq, HCl salt) in DMF (0.5 mL) was added a solution of HOBt (79.10 mg, 585.41 umol, 3 eq) and EDCI (112.22 mg, 585.41 umol, 3 eq) in DMF (0.5 mL), followed by TEA (118.47 mg, 1.17 mmol, 162.96 uL, 6 eq). The mixture was stirred at 25° C. for 1 hr. LC-MS showed that desired compound was detected. The reaction mixture was filtered. The filtrate was purified by prep-HPLC (column: Phenomenex Synergi C18 150*30 mm*4 um; mobile phase: A: 0.225% formic acid in water; B: CH₃CN; gradient:52%-82% over 11 min). Compound 5-(2-fluorophenyl)-N-(6-silaspiro[5.5]undecan-3-yl)-1H-pyrrolo[2,3-b]pyridine-2-carboxamide (11.1 mg, 25.54 umol, 13.09% yield, 97% purity) was obtained as a white solid.

LCMS m/z 422.1 [M+1]⁺; ¹H NMR (400 MHz, METHANOL-d4) 6=8.49 (s, 1H), 8.24 (s, 1H), 7.60-7.52 (m, 1H), 7.41 (q, J=6.9 Hz, 1H), 7.33-7.28 (m, 1H), 7.28-7.21 (m, 1H), 7.17 (s, 1H), 3.82 (br t, J=11.1 Hz, 1H), 2.17 (br d, J=12.7 Hz, 2H), 1.80-1.63 (m, 6H), 1.46 (br s, 2H), 0.99 (br d, J=13.9 Hz, 2H), 0.83-0.76 (m, 2H), 0.74-0.64 (m, 4H).

### Example 161. MPL-435

### Synthesis of 5-(3-pyridyl)-N-(6-silaspiro[5.5]undecan-3-yl)-1H-pyrrolo[2,3-b]pyridine-2-carboxamide

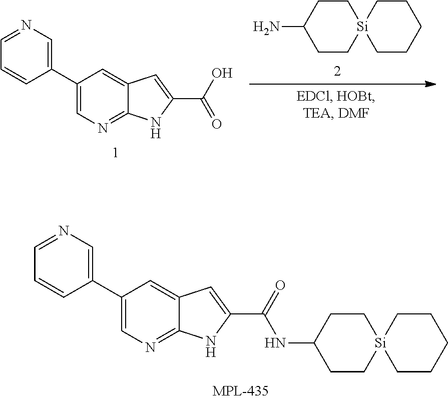 [SMILES: O=C(O)c1cc2cc(-c3cccnc3)cnc2[nH]1 ; NC1CC[Si]2(CCCCC2)CC1 ; O=C(NC1CC[Si]2(CCCCC2)CC1)c1cc2cc(-c3cccnc3)cnc2[nH]1]

To a solution of 5-(3-pyridyl)-1H-pyrrolo[2,3-b]pyridine-2-carboxylic acid (30 mg, 125.40 umol, 1 eq) (made from 5-bromo-1-(p-tolylsulfonyl)pyrrolo[2,3-b]pyridine (200 mg, 560.45 umol) via the same procedures described in Example 158) and 6-silaspiro[5.5]undecan-3-amine (27.57 mg, 125.40 umol, 1 eq, HCl salt) in DMF (0.5 mL) was added a solution of HOBt (50.83 mg, 376.20 umol, 3 eq) and EDCI (72.12 mg, 376.20 umol, 3 eq) in DMF (0.5 mL), followed by TEA (76.13 mg, 752.40 umol, 104.72 uL, 6 eq). The mixture was stirred at 25° C. for 1 hr. LC-MS showed that desired compound was detected. The reaction mixture was filtered and the filtrate was purified by prep-HPLC (column: Phenomenex Synergi C18 150*30 mm*4 um; mobile phase: A: 0.225% formic acid in water; B: CH3CN; gradient: 45%-75% over 11 min). Compound 5-(3-pyridyl)-N-(6-silaspiro[5.5]undecan-3-yl)-1H-pyrrolo[2,3-b]pyridine-2-carboxamide (2.9 mg, 7.10 umol, 5.66% yield, 99% purity) was obtained as a white solid.

LCMS m/z: 405.2 [M+1]⁺; ¹H NMR (400 MHz, METHANOL-d4) 6=8.87 (s, 1H), 8.63 (d, J=1.8 Hz, 1H), 8.56 (br d, J=4.6 Hz, 1H), 8.38 (d, J=2.0 Hz, 1H), 8.17 (br d, J=8.1 Hz, 1H), 7.57 (dd, J=5.0, 8.0 Hz, 1H), 7.21 (s, 1H), 3.82 (br t, J=11.1 Hz, 1H), 2.17 (br d, J=9.3 Hz, 2H), 1.78-1.63 (m, 6H), 1.46 (br s, 2H), 1.31 (t, J=7.4 Hz, 1H), 0.99 (br d, J=14.5 Hz, 2H), 0.83-0.74 (m, 2H), 0.73-0.63 (m, 4H).

### Example 162. MPL-453

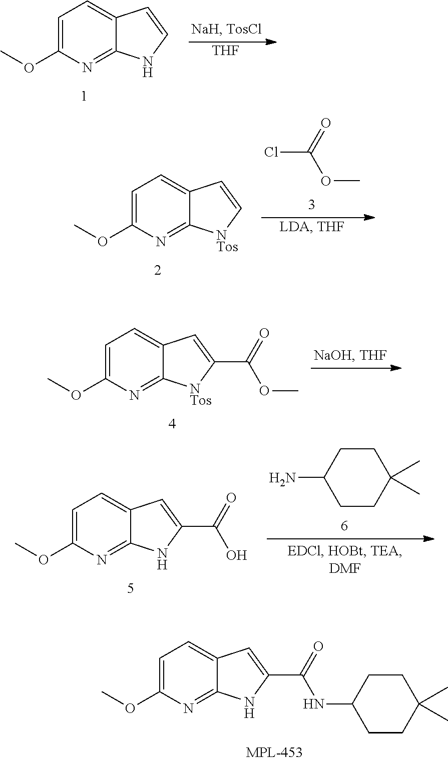

### Synthesis of 6-methoxy-1-(p-tolylsulfonyl)pyrrolo[2,3-b]pyridine

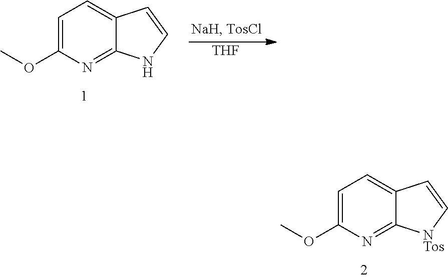

To a solution of 6-methoxy-1H-pyrrolo[2,3-b]pyridine (1 g, 6.75 mmol, 1 eq) in THE (15 mL) was added NaH (404.96 mg, 10.12 mmol, 60% purity, 1.5 eq). The mixture was stirred at 0° C. for 30 mins. TosCl (1.42 g, 7.42 mmol, 1.1 eq) was added. The mixture was stirred at 0° C. for 30 mins. TLC (Petroleum ether. Ethyl acetate=5:1) indicated starting material was consumed completely and many new spots formed. The reaction was quenched with saturated NH₄Cl (50 mL), then extracted with EtOAc (60 mL×2). The combined organic layer was washed with brine (50 mL×2), dried over Na₂SO₄, filtered and concentrated under reduced pressure. The residue was purified by column chromatography (SiO₂, Petroleum ether/Ethyl acetate=1/0 to 5/1). Compound 6-methoxy-1-(p-tolylsulfonyl)pyrrolo[2,3-b]pyridine (1.95 g, 5.80 mmol, 86.00% yield, 90% purity) was obtained as a white solid. ¹H NMR was recorded.

### Synthesis of methyl 6-methoxy-1-(p-tolylsulfonyl)pyrrolo[2,3-b]pyridine-2-carboxylate

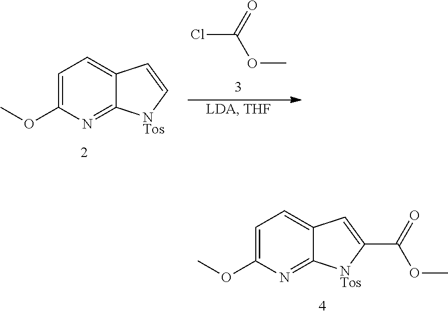

To a solution of 6-methoxy-1-(p-tolylsulfonyl)pyrrolo[2,3-b]pyridine (1.95 g, 6.45 mmol, 1 eq) in THE (20 mL) was added LDA (2 M in THF, 4.84 mL, 1.5 eq) dropwise at −78° C. under N₂. The reaction mixture was stirred at −78° C. for 30 mins. Methyl carbonochloridate (3.05 g, 32.25 mmol, 2.50 mL, 5 eq) (3.720 g) was added dropwise at −78° C. The reaction mixture was stirred at −78° C. for another 30 mins. TLC (petroleum ether:ethyl acetate=5:1) indicated new spots formed. The reaction mixture was quenched with saturated NH₄Cl (50 mL), extracted with dichloromethane (30 mL×3). The combined organic layer was washed with brine (30 mL), dried over Na₂SO₄, filtered and concentrated under reduced pressure. The resulting residue was purified by column chromatography (SiO₂, Petroleum ether/Ethyl acetate=1/0 to 0/1). Compound methyl 6-methoxy-1-(p-tolylsulfonyl)pyrrolo[2,3-b]pyridine-2-carboxylate (372 mg, 929.01 umol, 14.40% yield, 90% purity) was obtained as a white solid. ¹H NMR was recorded.

### Synthesis of 6-methoxy-1H-pyrrolo[2,3-b]pyridine-2-carboxylic acid

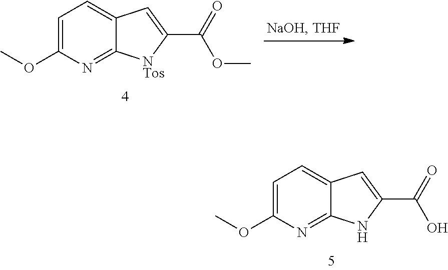

To a solution of methyl 6-methoxy-1-(p-tolylsulfonyl)pyrrolo[2,3-b]pyridine-2-carboxylate (372 mg, 1.03 mmol, 1 eq) in EtOH (3 mL) was added NaOH (2 M, 2.17 mL, 4.21 eq). The mixture was stirred at 80° C. for 2 hr. TLC (Petroleum ether:Ethyl acetate=5:1) indicated reactant was consumed completely and one new spot formed. The reaction mixture was concentrated under reduced pressure to remove EtOH. The aqueous phase was adjusted to pH to 3-4 with aqueous HCl (6N) and filtered. The cake was washed with petroleum ether (25 mL), dried under reduced pressure. Compound 6-methoxy-1H-pyrrolo[2,3-b]pyridine-2-carboxylic acid (115 mg, 568.50 umol, 55.08% yield, 95% purity) was obtained as a white solid, which was used for next step without further purification.

¹H NMR (400 MHz, DMSO-d6) δ=12.76 (br s, 1H), 12.15-11.93 (m, 1H), 7.95 (d, J=8.7 Hz, 1H), 7.03 (d, J=2.1 Hz, 1H), 6.61 (d, J=8.5 Hz, 1H), 3.94-3.87 (m, 3H).

### Synthesis of N-(4,4-dimethylcyclohexyl)-6-methoxy-1H-pyrrolo[2,3-b]pyridine-2-carboxamide

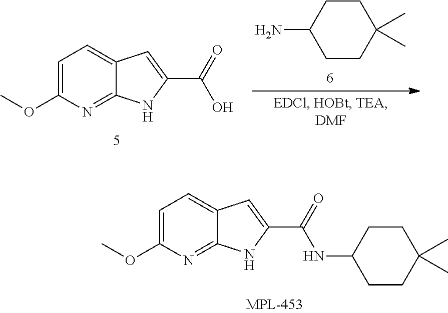

To a solution of 6-methoxy-1H-pyrrolo[2,3-b]pyridine-2-carboxylic acid (40 mg, 208.15 umol, 1 eq) and 4,4-dimethylcyclohexanamine (31.78 mg, 249.78 umol, 1.2 eq) in DMF (2 mL) was added a solution of EDCI (119.71 mg, 624.44 umol, 3 eq) and HOBt (84.37 mg, 624.44 umol, 3 eq) in DMF (1 mL), followed by TEA (105.31 mg, 1.04 mmol, 144.86 uL, 5 eq). The mixture was stirred at 25° C. for 1 hr. LC-MS showed desired product. The reaction mixture was filtered. The filtrate was purified by prep-HPLC (column: Phenomenex Synergi C18 150*30 mm*4 um; mobile phase: A: 0.225% formic acid in water; B: CH₃CN; gradient: 47%-77% B over 11 min). Compound N-(4,4-dimethylcyclohexyl)-6-methoxy-1H-pyrrolo[2,3-b]pyridine-2-carboxamide (43.1 mg, 143.01 umol, 68.70% yield, 100% purity) was obtained as a white solid.

LCMS m/z 302.1 [M+1]⁺; ¹H NMR (500 MHz, DMSO-d6) δ=11.83 (s, 1H), 8.03-7.86 (m, 2H), 7.02 (d, J=2.1 Hz, 1H), 6.57 (d, J=8.5 Hz, 1H), 3.88 (s, 3H), 3.76-3.65 (m, 1H), 1.71-1.62 (m, 2H), 1.56-1.44 (m, 2H), 1.41 (br d, J=12.7 Hz, 2H), 1.32-1.21 (m, 2H), 0.93 (d, J=10.1 Hz, 6H).

### Example 163. MPL-454

### Synthesis of 6-methoxy-N-(5-silaspiro[4.5]decan-8-yl)-1H-pyrrolo[2,3-b]pyridine-2-carboxamide

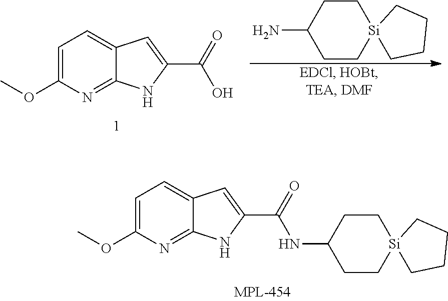

To a solution of 5-silaspiro[4.5]decan-8-amine (38.55 mg, 187.33 umol, 1.2 eq, HCl salt) and 6-methoxy-1H-pyrrolo[2,3-b]pyridine-2-carboxylic acid (30 mg, 156.11 umol, 1 eq) in DMF (1 mL) was added a solution of EDCI (89.78 mg, 468.33 umol, 3 eq) and HOBt (63.28 mg, 468.33 umol, 3 eq) in DMF (1 mL), followed by TEA (78.98 mg, 780.55 umol, 108.64 uL, 5 eq). The mixture was stirred at 25° C. for 1 hr. LC-MS showed desired mass. The reaction mixture was filtered. The filtrate was purified by prep-HPLC (column: Phenomenex Synergi C18 150*30 mm*4 um; mobile phase: A: 0.225% formic acid in water; B: CH₃CN; gradient: 55%-85% B over 11 min). Compound 6-methoxy-N-(5-silaspiro[4.5]decan-8-yl)-1H-pyrrolo[2,3-b]pyridine-2-carboxamide (24.5 mg, 71.33 umol, 45.69% yield, 100% purity) was obtained as a white solid.

LCMS m/z 344.1 [M+1]⁺; ¹H NMR (500 MHz, DMSO-d6) 3=11.84 (s, 1H), 7.98 (d, J=8.1 Hz, 1H), 7.92 (d, J=8.5 Hz, 1H), 7.02 (d, J=1.8 Hz, 1H), 6.57 (d, J=8.4 Hz, 1H), 3.88 (s, 3H), 3.79-3.70 (m, 1H), 2.10-2.00 (m, 2H), 1.66-1.48 (m, 6H), 0.86-0.77 (m, 2H), 0.77-0.67 (m, 2H), 0.61 (br t, J=6.7 Hz, 2H), 0.53 (br t, J=6.8 Hz, 2H).

### Example 164. MPL-455

### Synthesis of 6-methoxy-N-(6-silaspiro[5.5]undecan-3-yl)-1H-pyrrolo[2,3-b]pyridine-2-carboxamide

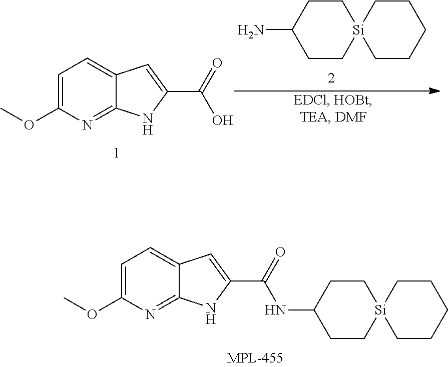 [SMILES: COc1ccc2cc(C(=O)O)[nH]c2n1 ; NC1CC[Si]2(CCCCC2)CC1 ; "EDCl, HOBt,TEA, DMF" ; COc1ccc2cc(C(=O)NC3CC[Si]4(CCCCC4)CC3)[nH]c2n1]

To a solution of 6-methoxy-1H-pyrrolo[2,3-b]pyridine-2-carboxylic acid (30 mg, 156.11 umol, 1 eq) and 6-silaspiro[5.5]undecan-3-amine (41.18 mg, 187.33 umol, 1.2 eq, HCl salt) in DMF (1 mL) was added a solution of EDCI (89.78 mg, 468.33 umol, 3 eq) and HOBt (63.28 mg, 468.33 umol, 3 eq) in DMF (1 mL), followed by TEA (78.98 mg, 780.55 umol, 108.64 uL, 5 eq). The mixture was stirred at 25° C. for 1 hr. LC-MS showed desired mass was detected. The reaction mixture was filtered. The residue was purified by prep-HPLC (column: Phenomenex Synergi C18 150*30 mm*4 um; mobile phase: A: 0.225% formic acid in water; B: CH₃CN; gradient: 55%-85% B over 11 min). Compound 6-methoxy-N-(6-silaspiro[5.5]undecan-3-yl)-1H-pyrrolo[2,3-b]pyridine-2-carboxamide (48.4 mg, 135.21 umol, 86.61% yield, 99.88% purity) was obtained as a white solid.

LCMS m/z 358.2 [M+1]⁺; ¹H NMR (400 MHz, DMSO-d6) δ=11.84 (s, 1H), 7.97 (d, J=8.2 Hz, 1H), 7.92 (d, J=8.6 Hz, 1H), 7.01 (d, J=2.0 Hz, 1H), 6.57 (d, J=8.6 Hz, 1H), 3.88 (s, 3H), 3.76-3.64 (m, 1H), 1.99 (br d, J=9.8 Hz, 2H), 1.73-1.48 (m, 6H), 1.38 (br s, 2H), 0.89 (br d, J=14.5 Hz, 2H), 0.74-0.65 (m, 2H), 0.63-0.51 (m, 4H).

### Example 165. MPL-465

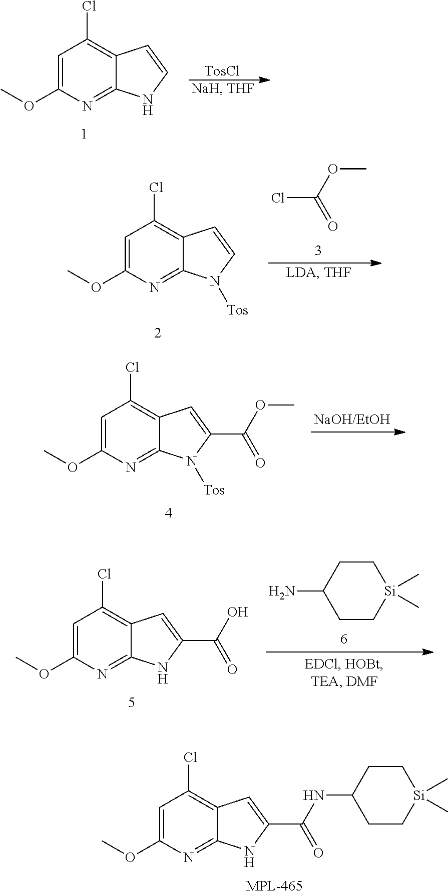

### Synthesis of ethyl (Z)-2-azido-3-[2-(2-methoxyethoxy)thiazol-5-yl]prop-2-enoate

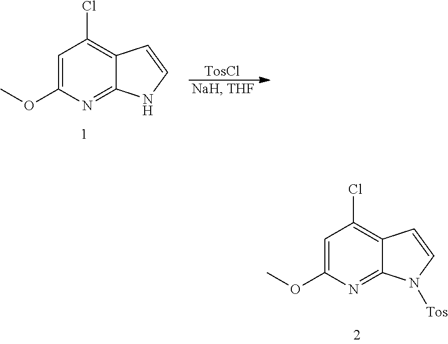

To a solution of 4-chloro-6-methoxy-1H-pyrrolo[2,3-b]pyridine (1 g, 5.48 mmol, 1 eq) in THE (20 mL) was added NaH (328.54 mg, 8.21 mmol, 60% purity, 1.5 eq). The mixture was stirred at 0° C. for 30 mins. Then TosCl (1.15 g, 6.02 mmol, 1.1 eq) was added. The mixture was stirred at 0° C. for 30 mins. TLC (Petroleum ether: Ethyl acetate=5:1) indicated starting material was consumed completely and new spot formed. The reaction was quenched with saturated NH₄Cl (50 mL), and then extracted with EtOAc (30 mL×3). The combined organic layer was washed with brine (50 mL×2), dried over Na₂SO₄, filtered and concentrated under reduced. The resulting residue was purified by column chromatography (SiO₂, 0-10% Ethyl acetate in petroleum ether). Compound 4-chloro-6-methoxy-1-(p-tolylsulfonyl) pyrrolo[2,3-b]pyridine (1.9 g, 5.08 mmol, 92.72% yield, 90% purity) was obtained as a white solid. ¹H NMR was recorded.

### Synthesis of methyl 4-chloro-6-methoxy-1-(p-tolylsulfonyl)pyrrolo[2,3-b]pyridine-2-carboxylate

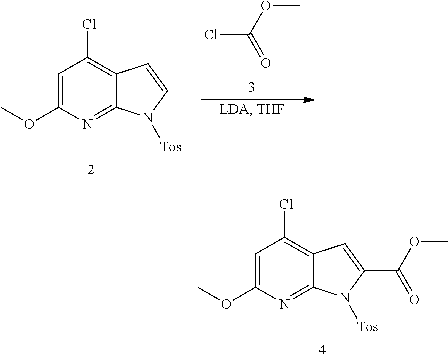

To a solution of 4-chloro-6-methoxy-1-(p-tolylsulfonyl)pyrrolo[2,3-b]pyridine (1.9 g, 5.64 mmol, 1 eq) in THE (20 mL) was added LDA (2 M in THF, 4.23 mL, 1.5 eq) drop-wise at −78° C. under N₂. The reaction mixture was stirred at −78° C. for 30 min. Methyl carbonochloridate (2.67 g, 28.21 mmol, 2.18 mL, 5 eq) (3.200 g) was added dropwise at −78° C. The reaction mixture was stirred at −78° C. for another 30 mins. TLC (Petroleum ether:Ethyl acetate=5:1) indicated new spots formed. The reaction mixture was quenched with saturated NH₄Cl (50 mL), extracted with dichloromethane (40 mL×3). The combined organic layer was washed with brine (60 mL), dried over Na₂SO₄, filtered and concentrated under reduced pressure. The resulting residue was purified by column chromatography (SiO₂, 0-20% Ethyl acetate in petroleum ether). Compound methyl 4-chloro-6-methoxy-1-(p-tolylsulfonyl) pyrrolo[2,3-b]pyridine-2-carboxylate (1.15 g, 2.77 mmol, 49.05% yield, 95% purity) was obtained as a white solid. ¹H NMR was recorded.

### Synthesis of 4-chloro-6-methoxy-1H-pyrrolo[2,3-b]pyridine-2-carboxylic acid

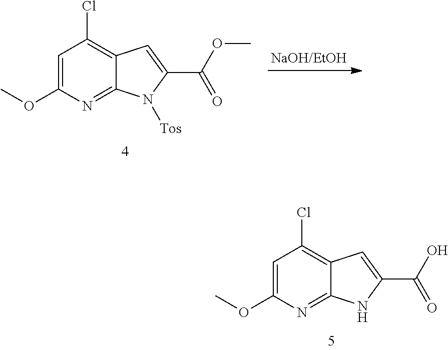

To a solution of methyl 4-chloro-6-methoxy-1-(p-tolylsulfonyl)pyrrolo[2,3-b]pyridine-2-carboxylate (1.15 g, 2.91 mmol, 1 eq) in EtOH (8 mL) was added NaOH (2 M in water, 8 mL, 5.49 eq). The mixture was stirred at 80° C. for 2 hr. TLC (Petroleum ether: Ethyl acetate=5:1) indicated starting material was consumed completely and one new spot formed. The reaction mixture was concentrated under reduced pressure to remove EtOH. The aqueous phase was adjusted to pH 3-4 with aqueous HCl (6 N), and then filtered. The cake was with petroleum ether (25 mL) and dried under reduced pressure to give 4-chloro-6-methoxy-1H-pyrrolo[2,3-b]pyridine-2-carboxylic acid (645 mg, 2.56 mmol, 87.95% yield, 90% purity) as a white solid. The crude product was used for the next step without further purification.

¹H NMR (400 MHz, DMSO-d₆) δ=12.72-12.07 (m, 1H), 6.98 (s, 1H), 6.80 (s, 1H), 3.91 (s, 3H)

### Synthesis of 4-chloro-N-(1,1-dimethylsilinan-4-yl)-6-methoxy-1H-pyrrolo[2,3-b]pyridine-2-carboxamide

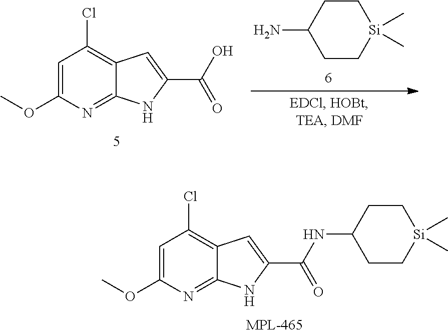

To a solution of 4-chloro-6-methoxy-1H-pyrrolo[2,3-b]pyridine-2-carboxylic acid (50 mg, 220.64 umol, 1 eq) and 1,1-dimethylsilinan-4-amine (47.59 mg, 264.77 umol, 1.2 eq, HCl salt) in DMF (1 mL) was added a solution of EDCI (126.89 mg, 661.91 umol, 3 eq) and HOBt (89.44 mg, 661.91 umol, 3 eq) in DMF (1 mL), followed by TEA (111.63 mg, 1.10 mmol, 153.55 uL, 5 eq). The mixture was stirred at 25° C. for 1 hr. LC-MS showed desired mass. The reaction mixture was filtered. The filterate was purified by prep-HPLC (column: Phenomenex Synergi C18 150*30 mm*4 um; mobile phase: A: 0.225% formic acid in water, B: CH₃CN; gradient 60%-90% B over 11 min). Compound 4-chloro-N-(1,1-dimethylsilinan-4-yl)-6-methoxy-1H-pyrrolo[2,3-b]pyridine-2-carboxamide (59.6 mg, 168.68 umol, 76.45% yield, 99.6% purity) was obtained as a white solid.

LCMS (ESI) m/z: 352.0 [M+H]⁺; ¹H NMR (400 MHz, DMSO-d6) δ=12.22 (s, 1H), 8.16 (d, J=7.8 Hz, 1H), 7.14 (s, 1H), 6.75 (s, 1H), 3.89 (s, 3H), 3.76-3.63 (m, 1H), 1.98 (br d, J=9.8 Hz, 2H), 1.66-1.48 (m, 2H), 0.77 (br d, J=14.5 Hz, 2H), 0.59 (dt, J=4.9, 14.0 Hz, 2H), 0.12-−0.01 (m, 6H).

### Example 166: MPL-466, MPL-466A and MPL-466B

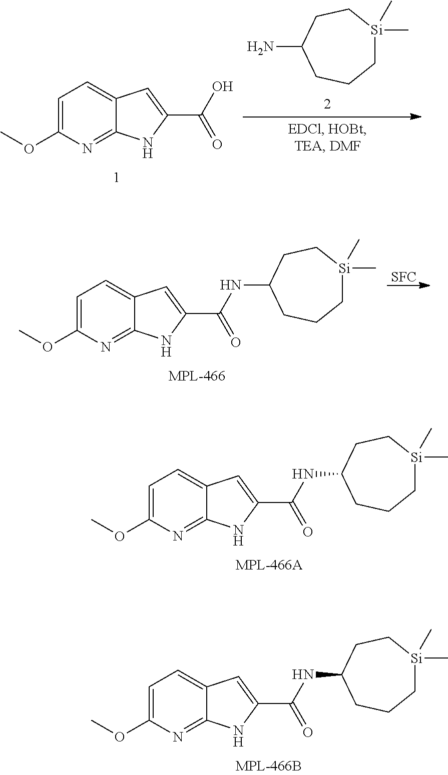

### Synthesis of N-(1,1-dimethylsilepan-4-yl)-6-methoxy-1H-pyrrolo[2,3-b]pyridine-2-carboxamide, (S)—N-(1,1-dimethylsilepan-4-yl)-6-methoxy-1H-pyrrolo[2,3-b]pyridine-2-carboxamide and (R)—N-(1,1-dimethylsilepan-4-yl)-6-methoxy-1H-pyrrolo[2,3-b]pyridine-2-carboxamide

To a solution of 6-methoxy-1H-pyrrolo[2,3-b]pyridine-2-carboxylic acid (30 mg, 156.11 umol, 1 eq) and 1,1-dimethylsilepan-4-amine (36.30 mg, 187.33 umol, 1.2 eq, HCl salt) in DMF (1 mL) was added a solution of EDCI (89.78 mg, 468.33 umol, 3 eq) and HOBt (63.28 mg, 468.33 umol, 3 eq) in DMF (1 mL), followed by TEA (78.98 mg, 780.55 umol, 108.64 uL, 5 eq). The mixture was stirred at 25° C. for 1 hr. LC-MS showed desired mass. The reaction mixture was filtered. The filtrate was purified by prep-HPLC (column: Phenomenex Synergi C18 150*30 mm*4 um; mobile phase: A: 0.225% formic acid in water, B: CH₃CN, gradient 52%-82% B over 11 min). Compound N-(1,1-dimethylsilepan-4-yl)-6-methoxy-1H-pyrrolo[2,3-b]pyridine-2-carboxamide (MPL-466) (25.7 mg, 77.53 umol, 49.66% yield, 100% purity) was obtained as a white solid.

LCMS (ESI) m/z: 332.2 [M+H]⁺; ¹H NMR (400 MHz, DMSO-d6) δ=11.83 (s, 1H), 8.00 (d, J=7.8 Hz, 1H), 7.92 (d, J=8.6 Hz, 1H), 7.02 (d, J=1.7 Hz, 1H), 6.57 (d, J=8.6 Hz, 1H), 3.88 (s, 4H), 1.97-1.74 (m, 3H), 1.72-1.60 (m, 1H), 1.56-1.42 (m, 2H), 0.81-0.68 (m, 2H), 0.67-0.54 (m, 2H), 0.03 (d, J=6.4 Hz, 6H).

The above reaction was conducted at a larger scale from 624.44 umol of compound 1, which was made using the same procedures described for the synthesis of compound 5 from compound 1 in Example 153. The racemic MPL-466 isolated from prep-HPLC was separated by SFC (Berger MG II, column: DAICEL CHIRALPAK AD (250 mm*30 mm, 10 um); mobile phase: A: 0.1% NH₃H₂O in MeOH; B: CO₂; isocratic 40% B; flow rate: 80 mL/min) to afford two peaks (two enantiomers), (S)—N-(1,1-dimethylsilepan-4-yl)-6-methoxy-1H-pyrrolo[2,3-b]pyridine-2-carboxamide and (R)—N-(1,1-dimethylsilepan-4-yl)-6-methoxy-1H-pyrrolo[2,3-b]pyridine-2-carboxamide.

Peak 1 (MPL-466A): 82.8 mg, 249.79 umol, 33.12% yield, 100% purity, a white solid.

LCMS m/z: 332.1 [M+1]⁺; ¹H NMR (400 MHz, DMSO-d6) δ=11.84 (s, 1H), 8.00 (d, J=7.8 Hz, 1H), 7.92 (d, J=8.6 Hz, 1H), 7.02 (d, J=2.0 Hz, 1H), 6.57 (d, J=8.2 Hz, 1H), 3.88 (s, 4H), 1.96-1.75 (m, 3H), 1.72-1.60 (m, 1H), 1.56-1.41 (m, 2H), 0.82-0.68 (m, 2H), 0.67-0.55 (m, 2H), 0.03 (d, J=6.3 Hz, 6H).

Peak 2 (MPL-466B): 92.4 mg, 277.64 umol, 36.81% yield, 99.60% purity, a white solid.

MPL-466A and MPL-466B were also analyzed by analytical SFC.

### Conditions:

Instrument: CAS-SH-ANA-SFC-K (Waters UPCC with PDA Detector)
Column: Chiralpak AD-3 50*4.6 mm, 3 um particle size
Mobile phase: A: CO₂, B: 0.05% DEA in methanol

### Isocratic: 40% B

Flow rate: 2.5 mL/min
Column temp.: 35° C.

### ABPR: 1500 psi

MPL-466A: retention time 2.53 min; 100% ee; MPL-466B: retention time: 3.55 min; 100% ee.

### Example 167. MPL-467

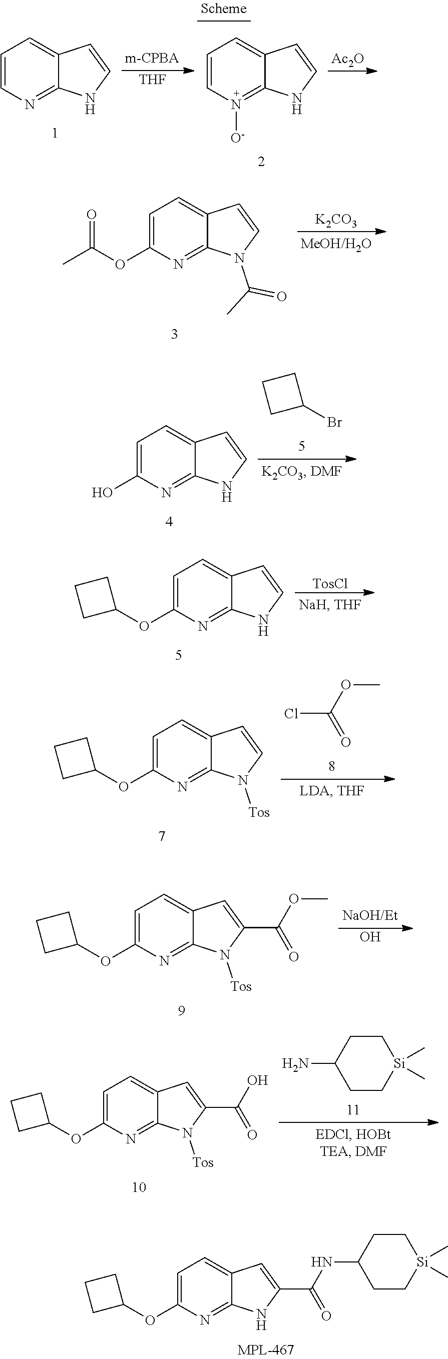

### Synthesis of 7-oxido-1H-pyrrolo

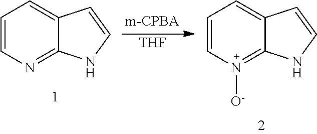

To a solution of 1H-pyrrolo[2,3-b]pyridine (9 g, 76.18 mmol, 1 eq) in THE (100 mL) was added 3-chlorobenzenecarboperoxoic acid (23.20 g, 114.28 mmol, 85% purity, 1.5 eq). The mixture was stirred at 25° C. for 12 hr. LC-MS showed desired mass was detected. The reaction mixture was diluted with Petroleum ether (200 mL), filtered and concentrated under reduced pressure afford 7-oxido-1H-pyrrolo[2,3-b]pyridin-7-ium (19 g, 70.82 mmol, 92.97% yield, 50% purity) as a white solid. The crude product was used in next step without further purification.

LCMS (ESI) m/z: 267.1 [M+H]⁺; ¹H NMR was recorded.

### Synthesis of (1-acetylpyrrolo[2,3-b]pyridin-6-yl) acetate

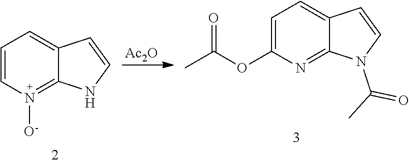

A mixture of 7-oxido-1H-pyrrolo[2,3-b]pyridin-7-ium (20 g, 74.55 mmol, 50% purity, 1 eq) in Ac₂O (107.39 g, 1.05 mol, 98.52 mL, 14.11 eq) was stirred at 140° C. for 12 hr. LC-MS showed desired mass. The reaction mixture was concentrated to half volume, and then extracted with CH₂Cl₂ (100 mL×2). The combined organic layer was washed with H₂O (100×2), dried over Na₂SO₄, filtered and concentrated under reduced pressure. The resulting residue was purified by column chromatography (SiO₂, 0-20% Ethyl acetate in petroleum ether). Compound (1-acetylpyrrolo[2,3-b]pyridin-6-yl) acetate (18 g, crude) was obtained as a white solid.

LCMS (ESI) m/z: 219.1 [M+H]⁺; ¹H NMR was recorded.

### Synthesis of 1H-pyrrolo[2,3-b]pyridin-6-ol

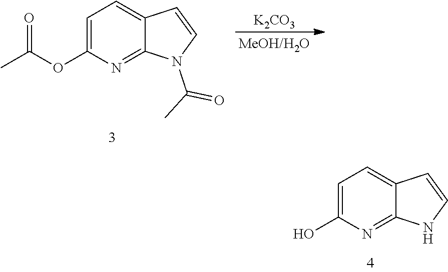

To a solution of (1-acetylpyrrolo[2,3-b]pyridin-6-yl) acetate (17 g, 77.91 mmol, 1 eq) in MeOH (30 mL) and H₂O (30 mL) was added K₂CO₃ (32.30 g, 233.72 mmol, 3 eq). The mixture was stirred at 25° C. for 12 hr. LC-MS showed desired mass. The reaction mixture was concentrated under reduced pressure to remove MeOH, and then filtered. The cake was dried under reduced pressure. Compound 1H-pyrrolo[2,3-b]pyridin-6-ol (6 g, crude) was obtained as a brown solid.

LCMS (ESI) m/z: 135.1 [M+H]⁺; ¹H NMR was recorded.

### Synthesis of 6-(cyclobutoxy)-1H-pyrrolo[2,3-b]pyridine

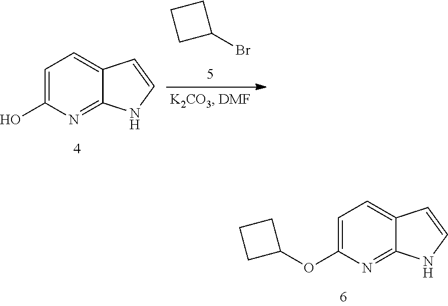 [SMILES: BrC1CCC1 ; Oc1ccc2cc[nH]c2n1 ; c1cc2ccc(OC3CCC3)nc2[nH]1]

To a solution of 1H-pyrrolo[2,3-b]pyridin-6-ol (2 g, 14.91 mmol, 1 eq) in DMF (20 mL) was added bromocyclobutane (2.42 g, 17.89 mmol, 1.69 mL, 1.2 eq) and K₂CO₃ (2.06 g, 14.91 mmol, 1 eq). The mixture was stirred at 80° C. for 12 hr. LC-MS showed desired mass. The residue was diluted with H₂O (20 mL) and extracted with EtOAc (30 mL×3). The combined organic layer was washed with H2O (30 mL×3), dried over Na₂SO₄, filtered and concentrated under reduced pressure. The resulting residue was purified by column chromatography (SiO₂, 0-10% Ethyl acetate in petroleum ether). Compound 6-(cyclobutoxy)-1H-pyrrolo[2,3-b]pyridine (400 mg, 1.91 mmol, 12.83% yield, 90% purity) was obtained as a white solid.

LCMS (ESI) m/z: 189.1 [M+H]⁺; ¹H NMR was recorded.

### Synthesis of 6-(cyclobutoxy)-1-(p-tolylsulfonyl) pyrrolo[2,3-b]pyridine

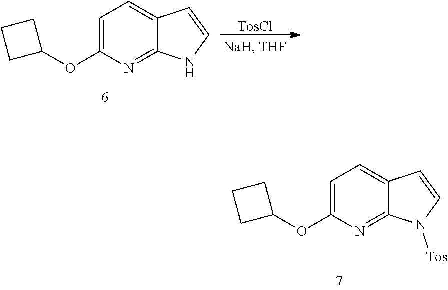

To a solution of 6-(cyclobutoxy)-1H-pyrrolo[2,3-b]pyridine (446 mg, 2.37 mmol, 1 eq) in THE (10 mL) was added NaH (142.16 mg, 3.55 mmol, 60% purity, 1.5 eq). The mixture was stirred at 0° C. for 30 mins. Then TosCl (496.91 mg, 2.61 mmol, 1.1 eq) was added. The mixture was stirred at 0° C. for 30 mins. TLC (Petroleum ether: Ethyl acetate=5:1) indicated starting material was consumed completely and new spot formed. The reaction was quenched with saturated NH₄Cl (30 mL), and then extracted with EtOAc (30 mL×3). The combined organic layer was washed with brine (50 mL×2), dried over Na₂SO₄, filtered and concentrated under reduced pressure. The resulting residue was purified by column chromatography (SiO₂, 0-10% Ethyl acetate in petroleum ether 1). Compound 6-(cyclobutoxy)-1-(p-tolylsulfonyl) pyrrolo[2,3-b]pyridine (680 mg, 1.79 mmol, 75.43% yield, 90% purity) was obtained as a white solid. 1H NMR was recorded.

### Synthesis of methyl 6-(cyclobutoxy)-1-(p-tolylsulfonyl)pyrrolo[2,3-b]pyridine-2-carboxylate

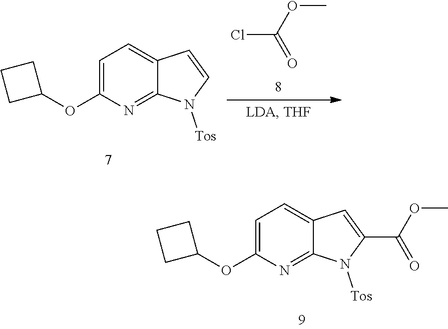

To a solution of 6-(cyclobutoxy)-1-(p-tolylsulfonyl)pyrrolo[2,3-b]pyridine (680 mg, 1.99 mmol, 1 eq) in THE (12 mL) was added LDA (2 M in THF, 1.49 mL, 1.5 eq) dropwise at −78° C. under N₂. The reaction mixture was stirred at −78° C. for 30 mins. Methyl carbonochloridate (938.31 mg, 9.93 mmol, 769.11 uL, 5 eq) was added dropwise at −78° C. The reaction mixture was stirred at −78° C. for another 30 mins. TLC (Petroleum ether:Ethyl acetate=5:1) indicated new spots formed. The reaction mixture was quenched with saturated NH₄Cl (30 mL), and then extracted with dichloromethane (40 mL×3). The combined organic layer was washed with brine (60 mL), dried over Na₂SO₄, filtered and concentrated under reduced pressure. The resulting residue was purified by column chromatography (SiO₂, 0-20% Ethyl acetate in petroleum ether). Compound methyl 6-(cyclobutoxy)-1-(p-tolylsulfonyl) pyrrolo[2,3-b]pyridine-2-carboxylate (245 mg, 550.63 umol, 27.73% yield, 90% purity) was obtained as a white solid. ¹H NMR was recorded.

### Synthesis of 6-(cyclobutoxy)-1H-pyrrolo[2,3-b]pyridine-2-carboxylicacid

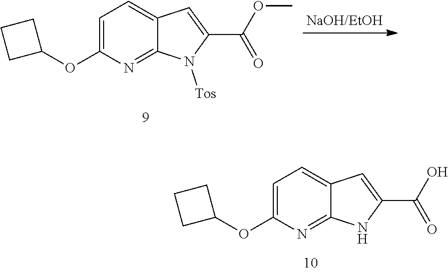

To a solution of methyl 6-(cyclobutoxy)-1-(p-tolylsulfonyl)pyrrolo[2,3-b]pyridine-2-carboxylate (1.17 g, 2.91 mmol, 1 eq) in EtOH (4 mL) was added NaOH (2 M in water, 4 mL, 2.75 eq). The mixture was stirred at 80° C. for 2 hr. TLC (Petroleum ether:Ethyl acetate=5:1) indicated starting material was consumed completely and one new spot formed. The reaction mixture was concentrated under reduced pressure to remove EtOH. The aqueous phase was adjust to pH 3-4 with aqueous HCl (6 N), and filtered. The cake was washed with petroleum ether (25 mL) and dried under reduced pressure. Compound 6-(cyclobutoxy)-1H-pyrrolo[2,3-b]pyridine-2-carboxylic acid (145 mg, 561.93 umol, 19.29% yield, 90% purity) was obtained as a white solid.

The crude product was used for the next step without further purification.

¹H NMR (500 MHz, DMSO-d₆) δ=13.38-12.28 (m, 1H), 12.07-11.83 (m, 1H), 7.94 (d, J=8.5 Hz, 1H), 7.01 (d, J=2.1 Hz, 1H), 6.56 (d, J=8.5 Hz, 1H), 5.17 (quin, J=7.3 Hz, 1H), 2.47-2.39 (m, 2H), 2.12-2.01 (m, 2H), 1.85-1.74 (m, 1H), 1.71-1.58 (m, 1H).

### Synthesis of 6-(cyclobutoxy)-N-(1,1-dimethylsilinan-4-yl)-1H-pyrrolo[2,3-b]pyridine-2-carboxamide

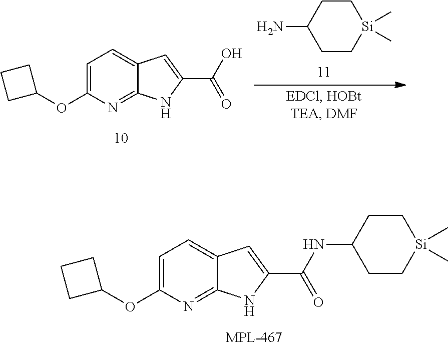

To a solution of 6-(cyclobutoxy)-1H-pyrrolo[2,3-b]pyridine-2-carboxylic acid (50 mg, 215.30 umol, 1 eq) and 1,1-dimethylsilinan-4-amine (46.44 mg, 258.36 umol, 1.2 eq, HCl salt) in DMF (1 mL) was added a solution of EDCI (123.82 mg, 645.90 umol, 3 eq) and HOBt (87.28 mg, 645.90 umol, 3 eq) in DMF (1 mL), followed by TEA (108.93 mg, 1.08 mmol, 149.83 uL, 5 eq). The mixture was stirred at 25° C. for 1 hr. LC-MS showed desired mass. The reaction mixture was filtered. The filtrate was purified by prep-HPLC (column: Phenomenex Synergi C18 150*30 mm*4 um; mobile phase: A: 0.225% formic acid in water, B: CH₃CN; gradient 58%-88% B over 11 min). Compound 6-(cyclobutoxy)-N-(1,1-dimethylsilinan-4-yl)-1H-pyrrolo[2,3-b]pyridine-2-carboxamide (52.5 mg, 146.84 umol, 68.20% yield, 100% purity) was obtained as a white solid.

LCMS (ESI) m/z: 358.3 [M+H]⁺; ¹1H NMR (500 MHz, DMSO-d6) 3=11.72 (s, 1H), 8.04-7.81 (m, 2H), 6.99 (d, J=2.0 Hz, 1H), 6.52 (d, J=8.5 Hz, 1H), 5.15 (quin, J=7.2 Hz, 1H), 3.75-3.63 (m, 1H), 2.48-2.38 (m, 2H), 2.12-1.93 (m, 4H), 1.79 (q, J=10.2 Hz, 1H), 1.71-1.50 (m, 3H), 0.77 (br d, J=14.5 Hz, 2H), 0.60 (dt, J=4.7, 14.0 Hz, 2H), 0.13-0.00 (m, 6H).

### Example 168: MPL-468

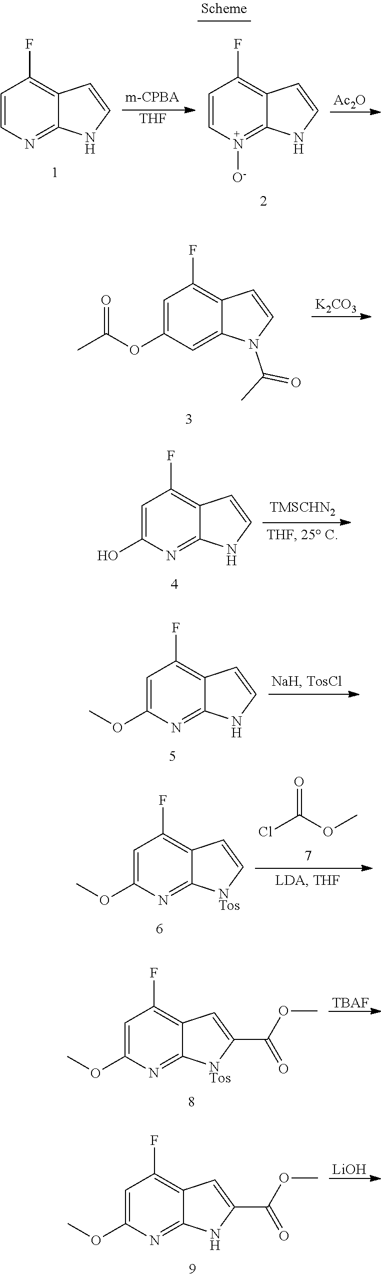

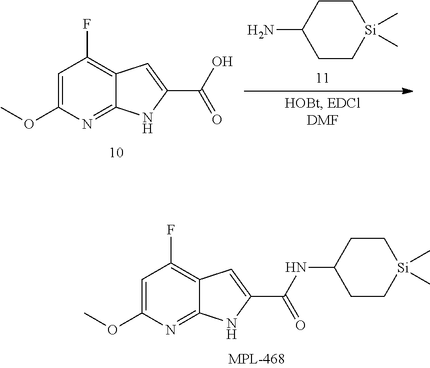

### Synthesis of 4-fluoro-7-oxido-1H-pyrrolo[2,3-b]pyridin-7-ium

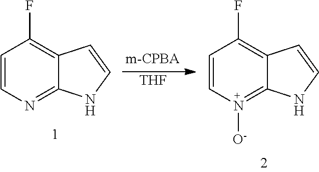

To a solution of 4-fluoro-1H-pyrrolo[2,3-b]pyridine (10 g, 73.46 mmol, 1 eq) in THF (150 mL) was added m-CPBA (18.22 g, 84.48 mmol, 80% purity, 1.15 eq) in batches. The mixture was stirred at 20° C. for 12 hr. TLC (Petroleum ether. EtOAc=3:1) showed starting material was consumed completely. The reaction mixture was poured into petroleum ether (500 mL), precipitates were collected by filtration. The cake was washed with petroleum ether (50 mL×2). The filtrate was quenched with Na₂SO₃ (Sat. 200 mL) and discarded. Compound 4-fluoro-7-oxido-1H-pyrrolo[2,3-b]pyridin-7-ium (20.2 g, 66.39 mmol, 90.38% yield, 50% purity) was obtained as a white solid. ¹H NMR was recorded.

### Synthesis of (1-acetyl-4-fluoro-pyrrolo[2,3-b]pyridin-6-yl) acetate

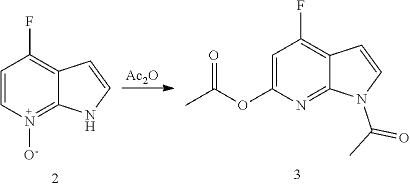

A solution of 4-fluoro-7-oxido-1H-pyrrolo[2,3-b]pyridin-7-ium (16 g, 52.59 mmol, 50% purity, 1 eq) in acetyl acetate (130.80 g, 1.28 mol, 120.00 mL, 24.36 eq) was stirred at 60° C. for 10 min. LCMS showed starting material was consumed completely and desired mass was detected. TLC (Petroleum ether:EtOAc=10:1) showed one major spot. The mixture was concentrated under reduced pressure. The residue was purified by flash silica gel chromatography (ISCO®; 330 g SepaFlash® Silica Flash Column, 0-10% Ethyl acetate in petroleum ether at 100 mL/min). Compound (1-acetyl-4-fluoro-pyrrolo[2,3-b]pyridin-6-yl) acetate (13.2 g, 16.77 mmol, 31.88% yield, 30% purity) was obtained as a colorless oil. ¹H NMR was recorded.

### Synthesis of 4-fluoro-1H-pyrrolo[2,3-b]pyridin-6-ol

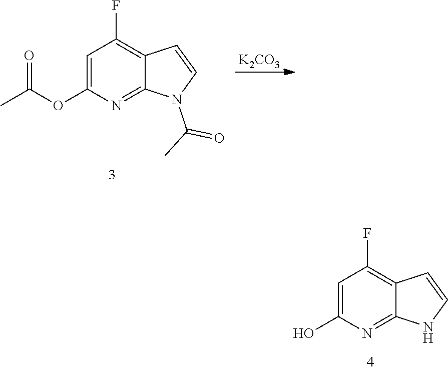

To a solution of (1-acetyl-4-fluoro-pyrrolo[2,3-b]pyridin-6-yl) acetate (13 g, 16.51 mmol, 30% purity, 1 eq) in MeOH (150 mL) and H₂O (50 mL) was added K₂CO₃ (9.13 g, 66.05 mmol, 4 eq). The mixture was stirred at 25° C. for 12 hr. LCMS showed starting material was consumed completely, and one peak with desired mass was detected. The reaction was quenched by dropwise addition of aqueous HCl (12 N) until pH=1. The mixture was diluted with H₂O (700 mL) to a solution, and then extracted by EtOAc (120 mL×5). The combined filtrate was dried over Na₂SO₄, filtered and concentrated under reduced pressure. The resulting residue was diluted with CH₃CN (10 mL) and water (40 mL), sonicated for 15 min and then filtered. The filter cake was dried under reduced pressure to afford crude compound 4 (1.5 g, 6.90 mmol, 41.80% yield, purity 70% purity) as a white solid. Additional amount of compound 4 (800 mg, 4.73 mmol, 28.66% yield, 90% purity) was obtained as a white solid after lyophilization of the filtrate. ¹H NMR was recorded.

### Synthesis of 4-fluoro-6-methoxy-1H-pyrrolo[2,3-b]pyridine

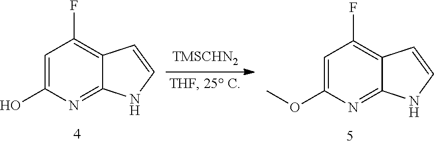

To a salt-ice cooled solution of 4-fluoro-1H-pyrrolo[2,3-b]pyridin-6-ol (1.4 g, 9.20 mmol, 1 eq) (from above 1.5 g of compound 4) in THF (150 mL) was added TMSCHN₂ (2 M in ether, 6.90 mL, 1.5 eq) dropwise and stirred at 50° C. for 6 hr under N₂. LCMS showed the starting material remained. The mixture was stirred at 50° C. for additional 12 h. TMSCHN₂ (2M in ether, 7 mL) was added and the mixture was stirred at 50° C. for another 8 h. LCMS showed the starting material remained. The mixture was stirred at 50° C. for another 12 h. LCMS showed the starting material remained. Additional TMSCHN₂ (2M in ether, 7 mL) was added. The mixture was stirred at 50° C. for another 8 h. LCMS showed one main peak with desired mass. The mixture was poured into saturated NH₄Cl (150 mL), and then extracted with EtOAc (100 mL×2). The combined organic layer was washed with brine (50 mL), dried over Na₂SO₄, filtered and concentrated under reduced pressure. The resulting residue was purified by flash silica gel chromatography (ISCO®; 20 g SepaFlash® Silica Flash Column, eluent of 0˜20% Ethyl acetate in petroleum ether at 40 mL/min). Compound 4-fluoro-6-methoxy-1H-pyrrolo[2,3-b]pyridine (900 mg, 5.15 mmol, 55.92% yield, 95% purity) was obtained as a white solid. ¹H NMR was recorded.

### Synthesis of 4-fluoro-6-methoxy-1-(p-tolylsulfonyl)pyrrolo[2,3-b]pyridine

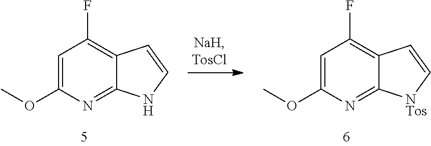

To an ice-cooled solution of 4-fluoro-6-methoxy-1H-pyrrolo[2,3-b]pyridine (900 mg, 5.42 mmol, 1 eq) in THF (15 mL) was added NaH (281.64 mg, 7.04 mmol, 60% purity, 1.3 eq) in batches, the mixture was stirred at 0° C. for 0.5 h. Then TosCl (1.14 g, 5.96 mmol, 1.1 eq) was added. The mixture was stirred at 0° C. for 0.5 hr. TLC (Petroleum ether:EtOAc=10:1) showed starting material was consumed completely, and one new spot formed. The reaction mixture was poured into saturated NH₄Cl (40 mL), and then extracted with EtOAc (20 mL×3). The combined organic layer was washed with brine (20 mL), dried over Na₂SO₄, filtered and concentrated under reduced pressure. The residue was purified by flash silica gel chromatography (ISCO®; 20 g SepaFlash® Silica Flash Column, eluent of 0-10% ethyl acetate in petroleum ether at 40 mL/min). Compound 4-fluoro-6-methoxy-1-(p-tolylsulfonyl)pyrrolo[2,3-b]pyridine (1.65 g, 4.89 mmol, 90.34% yield, 95% purity) was obtained as a white solid. ¹H NMR was recorded.

### Synthesis of methyl 4-fluoro-6-methoxy-1-(p-tolylsulfonyl)pyrrolo[2,3-b]pyridine-2-carboxylate

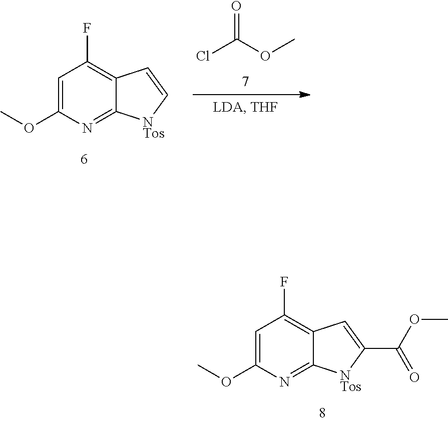

To a solution of 4-fluoro-6-methoxy-1-(p-tolylsulfonyl)pyrrolo[2,3-b]pyridine (500 mg, 1.56 mmol, 1 eq) in THE (10 mL) (dried by Na and distilled) was added LDA (2 M in THF, 1.17 mL, 1.5 eq) dropwsie at −70° C. under N₂. The mixture was stirred at −70˜−60° C. for 1 h. Methyl carbonochloridate (302.37 mg, 3.20 mmol, 247.84 uL, 2.05 eq) was added dropwise, the mixture was stirred at −70° C. for 1 hr. TLC (Petroleum ether:EtOAc=10:1) showed starting material was consumed completely, one new spot formed. The mixture was poured into saturated NH₄Cl (30 mL), and extracted with EtOAc (15 mL×2). The combined organic layer was washed with brine (15 mL), dried over Na₂SO₄, filtered and concentrated under reduced pressure to give a residue which was purified by flash silica gel chromatography (ISCO®; 12 SepaFlash® Silica Flash Column, Eluent of 0-15% ethyl acetate in petroleum ether at 40 mL/min). Compound methyl 4-fluoro-6-methoxy-1-(p-tolylsulfonyl)pyrrolo[2,3-b]pyridine-2-carboxylate (320 mg, 803.44 umol, 51.47% yield, 95% purity) was obtained as a white solid. ¹H NMR was recorded.

### Synthesis of methyl 4-fluoro-6-methoxy-1H-pyrrolo[2,3-b]pyridine-2-carboxylate

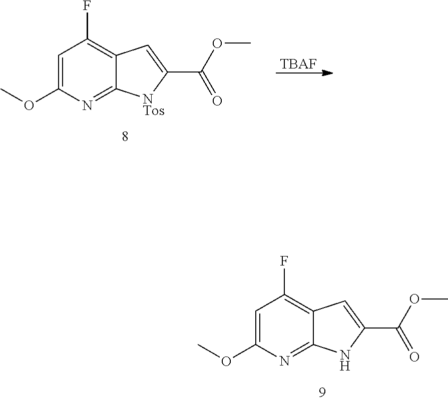

To a solution of methyl 4-fluoro-6-methoxy-1-(p-tolylsulfonyl)pyrrolo[2,3-b]pyridine-2-carboxylate (320 mg, 845.72 umol, 1 eq) in THE (5 mL) was added TBAF (1 M in THF, 1.01 mL, 1.2 eq). The mixture was stirred at 25° C. for 12 hr. TLC (Petroleum ether:EtOAc=3:1) showed starting material was consumed completely, and one new spot formed. The mixture was concentrated under reduced pressure. The resulting residue was diluted with water (10 mL) and sonicated for 15 min and filtered. The cake was washed with water (5 mL). Compound methyl 4-fluoro-6-methoxy-1H-pyrrolo[2,3-b]pyridine-2-carboxylate (190 mg, crude) was obtained as a white solid. It was used for the next step without further purification.

### Synthesis of 4-fluoro-6-methoxy-1H-pyrrolo[2,3-b]pyridine-2-carboxylic acid

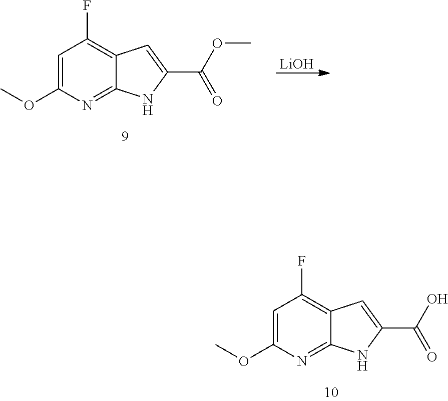

To a solution of methyl 4-fluoro-6-methoxy-1H-pyrrolo[2,3-b]pyridine-2-carboxylate (190 mg, 847.50 umol, 1 eq) in THE (3 mL) was added a solution of LiOH.H₂O (284.51 mg, 6.78 mmol, 8 eq) in H₂O (3 mL). The mixture was stirred at 25° C. for 2 hr. TLC (Petroleum ether:EtOAc=3:1) showed starting material was consumed completely, one new spot formed. The mixture was concentrated under reduced pressure. The aqueous residue was diluted with H₂O (5 mL), aqueous HCl (6 N) was added until pH to 3, and then filtered. The filtrate was concentrated by lyophilization. Compound 4-fluoro-6-methoxy-1H-pyrrolo[2,3-b]pyridine-2-carboxylic acid (195 mg, 835.07 umol, 98.53% yield, 90% purity).

¹H NMR (400 MHz, DMSO-d₆) δ=13.02 (br s, 1H), 12.45 (br s, 1H), 7.03 (d, J=1.8 Hz, 1H), 6.51 (d, J=11.3 Hz, 1H), 3.91 (s, 3H)

### Synthesis of N-(1,1-dimethylsilinan-4-yl)-4-fluoro-6-methoxy-1H-pyrrolo[2,3-b]pyridine-2-carboxamide

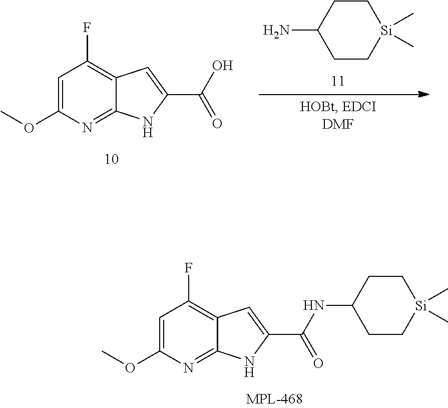

To a solution of 4-fluoro-6-methoxy-1H-pyrrolo[2,3-b]pyridine-2-carboxylic acid (50 mg, 237.91 umol, 1 eq) and 1,1-dimethylsilinan-4-amine (47.04 mg, 261.70 umol, 1.1 eq, HCl salt) in DMF (1 mL) was added a solution of HOBt (64.29 mg, 475.82 umol, 2 eq) and EDCI (91.22 mg, 475.82 umol, 2 eq) in DMF (1 mL), followed by TEA (96.30 mg, 951.65 umol, 132.46 uL, 4 eq). The mixture was stirred at 25° C. for 2 hr. LCMS showed starting material was consumed completely, and one major peak with desired mass was detected. The mixture was filtered to remove insoluble matter. The filtrate was purified by prep-HPLC (column: Welch Xtimate 75*40 mm*3 um; mobile phase: A: 0.225% formic acid in water, B: CH₃CN; gradient 55%-85% B over 10 min). Compound N-(1,1-dimethylsilinan-4-yl)-4-fluoro-6-methoxy-1H-pyrrolo[2,3-b]pyridine-2-carboxamide (32 mg, 95.39 umol, 40.10% yield, 100% purity) was obtained as a white solid.

LCMS (ESI) m/z 336.3 [M+H]⁺; ¹H NMR (400 MHz, DMSO-d₆) δ=12.20 (br s, 1H), 8.08 (d, J=8.1 Hz, 1H), 7.11 (s, 1H), 6.47 (d, J=11.3 Hz, 1H), 3.89 (s, 3H), 3.75-3.61 (m, 1H), 2.04-1.91 (m, 2H), 1.64-1.50 (m, 2H), 0.77 (br d, J=14.6 Hz, 2H), 0.59 (dt, J=4.8, 13.9 Hz, 2H), 0.12-0.02 (m, 6H).

### Example 169. MPL-469

### Synthesis of N-(1,1-dimethylsilocan-5-yl)-6-methoxy-1H-pyrrolo[2,3-b]pyridine-2-carboxamide

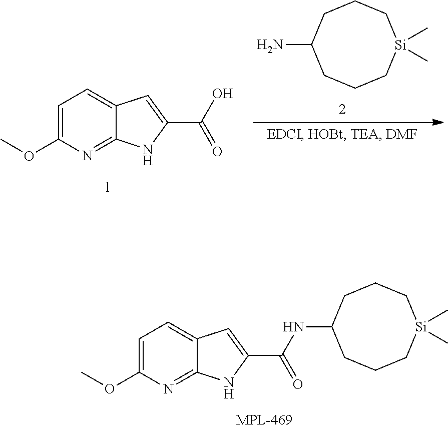

To a solution of 6-methoxy-1H-pyrrolo[2,3-b]pyridine-2-carboxylic acid (40 mg, 208.15 umol, 1 eq) and 1,1-dimethylsilocan-5-amine (51.91 mg, 249.78 umol, 1.2 eq, HCl salt) in DMF (1 mL) was added a solution of EDCI (119.71 mg, 624.44 umol, 3 eq) and HOBt (84.38 mg, 624.44 umol, 3 eq) in DMF (1 mL), followed by TEA (105.31 mg, 1.04 mmol, 144.86 uL, 5 eq). The mixture was stirred at 25° C. for 1 hr. LC-MS showed desired mass. The reaction mixture was filtered. The residue was purified by prep-HPLC (column: Phenomenex Synergi C18 150*30 mm*4 um; mobile phase: A: 0.225% formic acid in water, B: CH₃CN; gradient 60%-90% B over 11 min). The residue from prep-HPLC was further purified by SFC (Instrument: Berger MG II; column: DAICEL CHIRALPAK AS (250 mm*30 mm, 10 um); mobile phase: A: 0.1% NH₃H₂O in EtOH; B CO₂, isocratic 30% B). Compound N-(1,1-dimethylsilocan-5-yl)-6-methoxy-1H-pyrrolo[2,3-b]pyridine-2-carboxamide (7 mg, 20.26 umol, 35.00% yield, 100% purity) was obtained as a white solid.

LCMS (ESI) m/z: 346.1 [M+H]⁺; ¹H NMR (400 MHz, DMSO-d6) δ=11.80 (s, 1H), 8.05 (d, J=8.1 Hz, 1H), 7.92 (d, J=8.6 Hz, 1H), 7.02 (d, J=1.7 Hz, 1H), 6.57 (d, J=8.6 Hz, 1H), 4.08-3.96 (m, 1H), 3.88 (s, 3H), 1.73-1.57 (m, 8H), 0.80-0.64 (m, 4H), 0.03 (d, J=18.8 Hz, 6H).

### Example 170. MPL-471

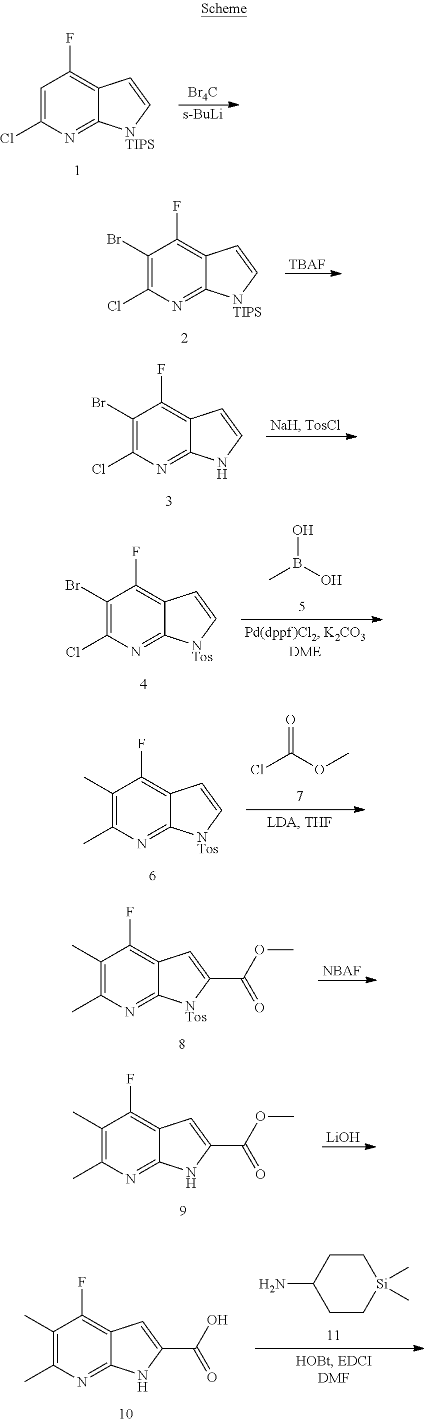

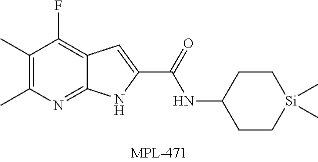

### Synthesis of (5-bromo-6-chloro-4-fluoro-pyrrolo[2,3-b]pyridin-1-yl)-triisopropyl-silane

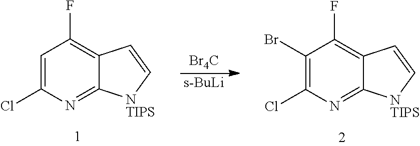

To a solution of (6-chloro-4-fluoro-pyrrolo[2,3-b]pyridin-1-yl)-triisopropyl-silane (9 g, 27.53 mmol, 1 eq) in THE (270 mL) was added n-BuLi (2.5 M in n-hexane, 22.02 mL, 2 eq) dropwise under N₂ at −78° C. The mixture was stirred at −70° C.˜−60° C. for 1 hr. Then a solution of carbon tetrabromide (22.82 g, 68.83 mmol, 2.5 eq) in THE (30 mL) was added dropwise. The reaction mixture was stirred at −70° C.˜−60° C. for 1 hr. LC-MS showed reactant was consumed completely and one main peak with desired mass was detected. The mixture was poured into saturated NH₄Cl (700 mL), and extracted with EtOAc (200 mL×2). The combined organic layer was washed with brine (100 mL), dried over Na₂SO₄, filtered and concentrated under reduced pressure. The resulting residue purified by flash silica gel chromatography (ISCO®; 330 g SepaFlash® Silica Flash Column; 0-3% ethyl acetate in petroleum ether at 100 mL/min). Compound (5-bromo-6-chloro-4-fluoro-pyrrolo[2,3-b]pyridin-1-yl)-triisopropyl-silane (15 g, 25.87 mmol, 93.98% yield, 70% purity) was obtained as a light yellow oil. ¹H NMR was recorded.

### Synthesis of 5-bromo-6-chloro-4-fluoro-1H-pyrrolo[2,3-b]pyridine

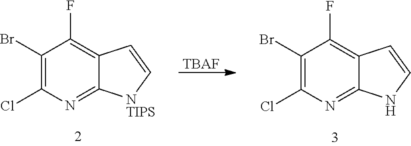

To a solution of (5-bromo-6-chloro-4-fluoro-pyrrolo[2,3-b]pyridin-1-yl)-triisopropyl-silane (16 g, 39.43 mmol, 1 eq) in THE (20 mL) was added TBAF (1 M in THF, 47.31 mL, 1.2 eq). The mixture was stirred at 20° C. for 3 hr. LCMS showed starting material was consumed completely, and one major peak with desired mass was detected. The mixture was poured into water (700 mL) with stirring, and then extracted with EtOAc (200 mL×3). The combined organic layer was washed with brine (100 mL), dried by Na₂SO₄, filtered and concentrated under reduced pressure. The resulting residue was diluted with a mixture of petroleum ether/EtOAc (20:1, 100 mL) and sonicated for 15 min before filtration. The cake was washed with petroleum ether/EtOAc (10:1, 20 mL×2) and dried to afford 5-bromo-6-chloro-4-fluoro-1H-pyrrolo[2,3-b]pyridine (3.1 g, 11.18 mmol, 28.37% yield, 90% purity) as a yellow solid. ¹H NMR was recorded.

The combined filtrate was concentrated under reduced pressure, the resulting residue was purified by flash silica gel chromatography (ISCO®; 40 g SepaFlash® Silica Flash Column, Eluent 0-40% ethyl acetate in petroleum ether at 50 mL/min) to afford additional amount of desire product (0.9 g, 3.43 mmol, 8.69% yield, 95% purity) as a yellow solid. ¹H NMR was recorded.

### Synthesis of 5-bromo-6-chloro-4-fluoro-1-(p-tolylsulfonyl)pyrrolo[2,3-b]pyridine

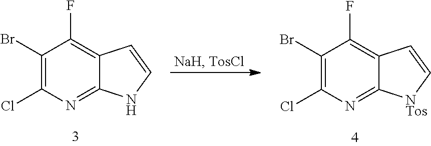

To an ice-cooled solution of 5-bromo-6-chloro-4-fluoro-1H-pyrrolo[2,3-b]pyridine (500 mg, 2.00 mmol, 1 eq) in THE (10 mL) was added NaH (120.24 mg, 3.01 mmol, 60% purity, 1.5 eq) at 0° C. in batches. The mixture was stirred at 0° C. for 0.5 hr. TosCl (458.53 mg, 2.41 mmol, 1.2 eq) was added. The mixture was stirred at 0° C. for 0.5 hr. TLC (Petroleum ether:EtOAc=10:1) showed starting material was consumed completely and one new spot formed. The mixture was poured into saturated NH₄Cl (30 mL), and then extracted with EtOAc (10 mL×2). The combined organic layer was washed with brine (10 mL), dried over Na₂SO₄, filtered and concentrated under reduced pressure. The resulting residue was purified by flash silica gel chromatography (ISCO®; 20 SepaFlash® Silica Flash Column, Eluent of 0-5% ethyl acetate in petroleum ether at 50 mL/min). Compound 5-bromo-6-chloro-4-fluoro-1-(p-tolylsulfonyl)pyrrolo[2,3-b]pyridine (450 mg, 1.06 mmol, 52.84% yield, 95% purity) was obtained as a white solid. ¹H NMR was recorded.

### Synthesis of 4-fluoro-5,6-dimethyl-1-(p-tolylsulfonyl)pyrrolo[2,3-b]pyridine

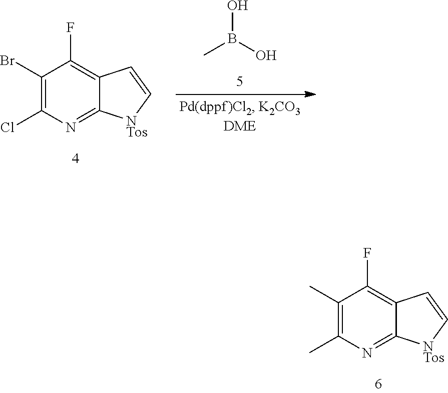

To a mixture of 5-bromo-6-chloro-4-fluoro-1-(p-tolylsulfonyl)pyrrolo[2,3-b]pyridine (300 mg, 743.21 umol, 1 eq), MeB(OH)₂ (444.89 mg, 7.43 mmol, 10 eq) and K₂CO₃ (308.15 mg, 2.23 mmol, 3 eq) was added DME (20 mL). The mixture was purged with N₂ and then Pd(dppf)Cl₂.CH₂Cl₂ (121.39 mg, 148.64 umol, 0.2 eq) was added under N₂. The mixture was stirred at 110° C. for 12 hr under N₂. TLC (Petroleum ether:EtOAc=10:1) showed starting material was consumed completely, and one new spot with desired mass was detected. The mixture was concentrated under reduced pressure. The resulting residue was purified by flash silica gel chromatography (ISCO®; 12 g SepaFlash® Silica Flash Column, Eluent 0-10% ethyl acetate in petroleum ether at 25 mL/min). Compound 4-fluoro-5,6-dimethyl-1-(p-tolylsulfonyl)pyrrolo[2,3-b]pyridine (270 mg, 720.87 umol, 96.99% yield, 85% purity) was obtained as a white solid. ¹H NMR was recorded.

### Synthesis of methyl 4-fluoro-5,6-dimethyl-1-(p-tolylsulfonyl)pyrrolo[2,3-b]pyridine-2-carboxylate

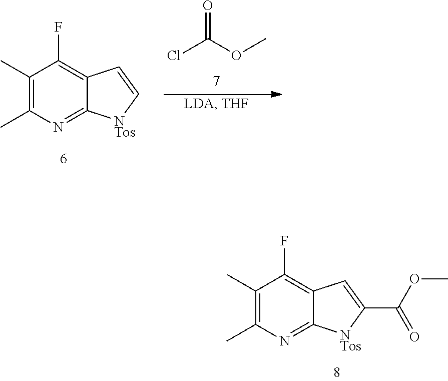

To a solution of 4-fluoro-5,6-dimethyl-1-(p-tolylsulfonyl)pyrrolo[2,3-b]pyridine (270 mg, 848.08 umol, 1 eq) in THE (5 mL) (dried by Na and distilled) was added LDA (2 M in THF, 636.06 uL, 1.5 eq) dropwise at −70° C. under N₂. After stirring at −70° C.˜−60° C. for 1 hr, methyl carbonochloridate (240.42 mg, 2.54 mmol, 197.07 uL, 3 eq) was added dropwise. The mixture was stirred at −70˜−60° C. for 1 hr. TLC (Petroleum ether:EtOAc=10:1) showed a little starting material remained and one major new spot formed. The mixture was poured into saturated NH₄Cl (25 mL), and then extracted by EtOAc (10 mL×2). The combined organic layer was washed with brine (10 mL), dried over Na₂SO₄, filtered and concentrated under reduced pressure. The resulting residue was purified by flash silica gel chromatography (ISCO®; 12 g SepaFlash® Silica Flash Column, Eluent 0-15% ethyl acetate in petroleum ether at 25 mL/min). Compound methyl 4-fluoro-5,6-dimethyl-1-(p-tolylsulfonyl)pyrrolo[2,3-b]pyridine-2-carboxylate (180 mg, 454.30 umol, 53.57% yield, 95% purity) was obtained as a white solid. ¹H NMR was recorded.

### Synthesis of methyl 4-fluoro-5,6-dimethyl-1H-pyrrolo[2,3-b]pyridine-2-carboxylate

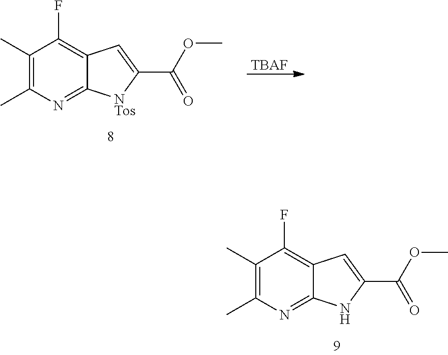

To a solution of methyl 4-fluoro-5,6-dimethyl-1-(p-tolylsulfonyl)pyrrolo[2,3-b]pyridine-2-carboxylate (180 mg, 478.21 umol, 1 eq) in THE (2 mL) was added TBAF (1 M in THF, 526.03 uL, 1.1 eq). The mixture was stirred at 25° C. for 2 hr. TLC (Petroleum ether:EtOAc=10:1) showed starting material was consumed completely, one major new spot formed. The mixture was concentrated under reduced pressure. The resulting residue was diluted with water (5 mL) and sonicated for 15 min before filtration. The cake was collected and washed with H₂O (2 mL). Compound methyl 4-fluoro-5,6-dimethyl-1H-pyrrolo[2,3-b]pyridine-2-carboxylate (100 mg, crude) was obtained as a brown solid.

### Synthesis of 4-fluoro-5,6-dimethyl-1H-pyrrolo[2,3-b]pyridine-2-carboxylic acid

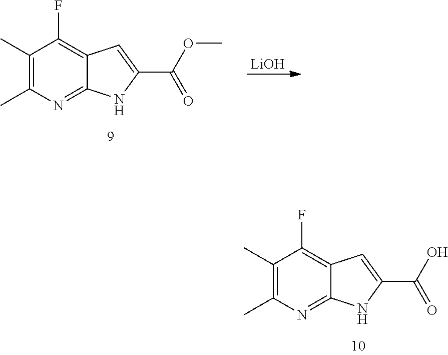

To a solution of methyl 4-fluoro-5,6-dimethyl-1H-pyrrolo[2,3-b]pyridine-2-carboxylate (100 mg, 450.01 umol, 1 eq) in THE (2 mL) and H₂O (2 mL) was added LiOH.H₂O (169.96 mg, 4.05 mmol, 9 eq). The mixture was stirred at 25° C. for 12 hr. TLC (Petroleum ether:EtOAc=3:1) showed starting material was consumed completely, and one new spot formed. The mixture was concentrated under reduced pressure to remove THF. The pH of aqueous phase was adjusted to 3 with aqueous HCl (6 N). The solid was collected by filtration. Compound 4-fluoro-5,6-dimethyl-1H-pyrrolo[2,3-b]pyridine-2-carboxylic acid (100 mg, 432.30 umol, 96.06% yield, 90% purity) was obtained as a white solid.

¹H NMR (400 MHz, DMSO-d₆) δ=12.30 (br s, 1H), 6.98 (s, 1H), 2.53 (s, 3H), 2.22 (s, 3H)

### Synthesis of N-(1,1-dimethylsilinan-4-yl)-4-fluoro-5,6-dimethyl-1H-pyrrolo[2,3-b]pyridine-2-carboxamide

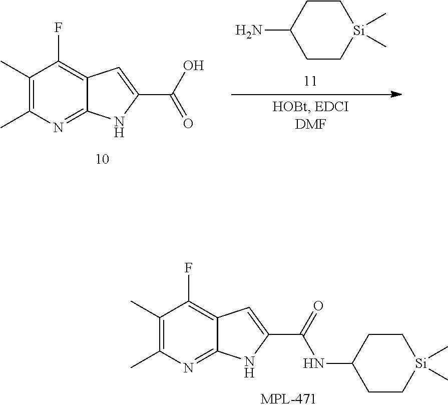

To a solution of 4-fluoro-5,6-dimethyl-1H-pyrrolo[2,3-b]pyridine-2-carboxylic acid (30 mg, 144.10 umol, 1 eq) and 1,1-dimethylsilinan-4-amine (31.08 mg, 172.92 umol, 1.2 eq, HCl salt) in DMF (1 mL) was added a solution of EDCI (55.25 mg, 288.20 umol, 2 eq) and HOBt (38.94 mg, 288.20 umol, 2 eq) in DMF (1 mL), followed by TEA (58.33 mg, 576.40 umol, 80.23 uL, 4 eq). The mixture was stirred at 25° C. for 2 hr. LCMS showed starting material was consumed completely, and one major peak with desired mass was detected. The reaction was filtered to remove insoluble matter. The filtrate was purified by prep-HPLC (column: Phenomenex luna C18 100*40 mm*3 um; mobile phase: A: 0.225% formic acid in water, B: CH₃CN; gradient: 55%-85% B over 11 min). Compound N-(1,1-dimethylsilinan-4-yl)-4-fluoro-5,6-dimethyl-1H-pyrrolo[2,3-b]pyridine-2-carboxamide (20 mg, 59.97 umol, 50.00% yield, 100% purity) was obtained as a white solid.

LCMS (ESI) m/z 334.3 [M+H]⁺; ¹H NMR (400 MHz, DMSO-d₆) δ=12.06 (br s, 1H), 8.20 (d, J=8.1 Hz, 1H), 7.10 (s, 1H), 3.76-3.62 (m, 1H), 2.50 (s, 3H), 2.21 (d, J=1.1 Hz, 3H), 2.05-1.91 (m, 2H), 1.66-1.51 (m, 2H), 0.77 (br d, J=14.5 Hz, 2H), 0.59 (dt, J=4.8, 14.1 Hz, 2H), 0.13-−0.03 (m, 6H).

### Example 171. MPL-351

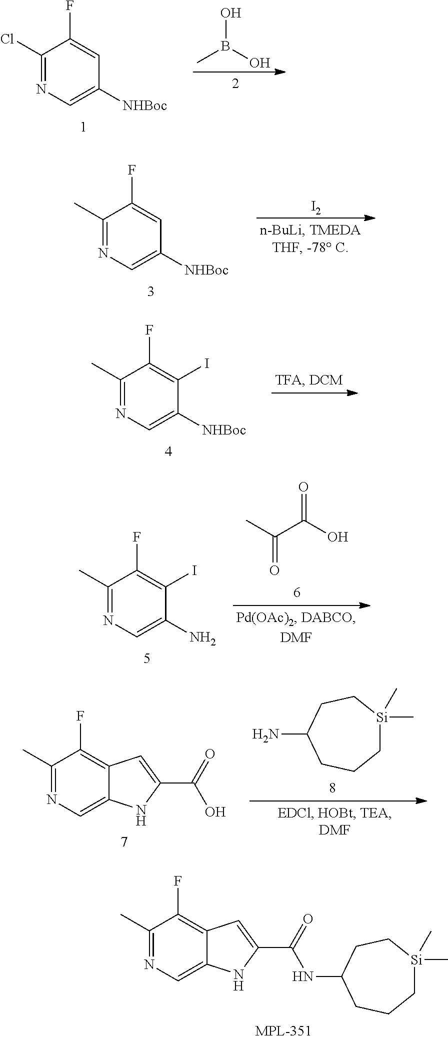

### Synthesis of tert-butyl N-(5-fluoro-6-methyl-3-pyridyl)carbamate

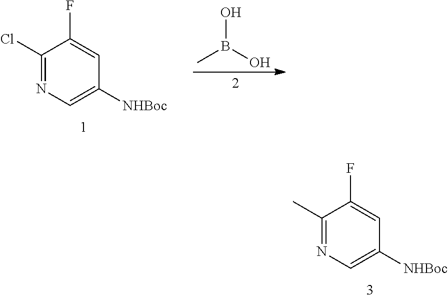

A mixture of tert-butyl N-(6-chloro-5-fluoro-3-pyridyl)carbamate (1.8 g, 7.30 mmol, 1 eq), methylboronic acid (2.18 g, 36.49 mmol, 5 eq) and Cs₂CO₃ (7.13 g, 21.89 mmol, 3 eq) in H₂O (0.1 mL) and dioxane (10 mL) was de-gassed and Pd(dppf)Cl₂ (533.95 mg, 729.73 umol, 0.1 eq) was then added. The mixture was heated at 100° C. for 12 hours under N₂. LC-MS indicated desired mass. The reaction mixture was diluted with EtOAc (30 mL) and filtered to remove the insoluble material. The filtrate was concentrated in vacuo. The residue was purified by flash silica gel chromatography (SiO₂, 0-30% ethyl acetate in petroleum ether). Compound tert-butyl N-(5-fluoro-6-methyl-3-pyridyl)carbamate (1.19 g, 5.00 mmol, 62.25% yield, 95% purity) was obtained as a yellow solid.

LCMS (ESI) m/z: 227.1 [M+H]⁺; ¹H NMR was recorded.

### Synthesis of tert-butyl N-(5-fluoro-4-iodo-6-methyl-3-pyridyl)carbamate

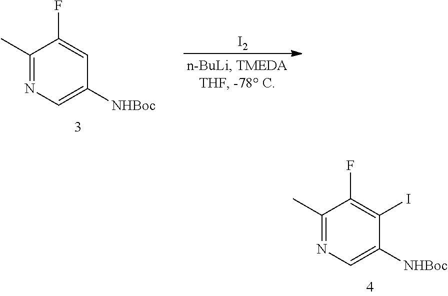 [SMILES: Cc1ncc(NC(=O)OC(C)(C)C)cc1F ; "I2" ; Cc1ncc(NC(=O)OC(C)(C)C)c(I)c1F]

To a solution of tert-butyl N-(5-fluoro-6-methyl-3-pyridyl)carbamate (1.19 g, 5.26 mmol, 1 eq) and TMEDA (1.22 g, 10.52 mmol, 1.59 mL, 2 eq) in THE (10 mL) was added n-BuLi (2.5 M in n-hexane, 5.26 mL, 2.5 eq) dropwise at −78° C. under N₂. After stirring at −78° C. for 30 min, a solution of I₂ (2.00 g, 7.89 mmol, 1.59 mL, 1.5 eq) in THE (3 mL) was added dropwise at −78° C. The reaction mixture was stirred at −78° C. for another 30 min. TLC (petroleum ether:ethyl acetate=3:1) indicated trace of starting material remained and new spots formed. The reaction mixture was quenched with saturated Na₂SO₃ (10 mL) at 25° C., and then diluted with H₂O (5 mL) and extracted with EtOAc (30 mL×2). The combined organic layer was washed with brine (20 mL), dried over Na₂SO₄, filtered and concentrated under reduced pressure. The resulting residue was purified by column chromatography (SiO₂, 0-20% ethyl acetate in petroleum ether). Compound tert-butyl N-(5-fluoro-4-iodo-6-methyl-3-pyridyl) carbamate (1.5 g, 3.83 mmol, 72.89% yield, 90% purity) was obtained as a white solid. ¹H NMR was recorded.

### Synthesis of 5-fluoro-4-iodo-6-methyl-pyridin-3-amine

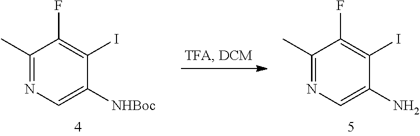

To a solution of tert-butyl N-(5-fluoro-4-iodo-6-methyl-3-pyridyl) carbamate (1.5 g, 4.26 mmol, 1 eq) in DCM (15 mL) was added TFA (23.10 g, 202.59 mmol, 15 mL, 47.56 eq). The mixture was stirred at 25° C. for 12 hr. LC-MS indicated desired mass. The reaction mixture was concentrated under reduced pressure. The residue was dissolved in sat. NaHCO₃ (5 mL), and then extracted with ethyl acetate (15 mL×2). The combined organic layer was washed with brine (15 mL), dried over Na₂SO₄, filtered and concentrated under reduced pressure. The resulting residue was purified by column chromatography (SiO₂, 0-30% ethyl acetate in petroleum ether). Compound 5-fluoro-4-iodo-6-methyl-pyridin-3-amine (956 mg, 3.60 mmol, 84.60% yield, 95% purity) was obtained as a brown solid. ¹H NMR was recorded.

### Synthesis of 4-fluoro-5-methyl-1H-pyrrolo[2,3-c]pyridine-2-carboxylic acid

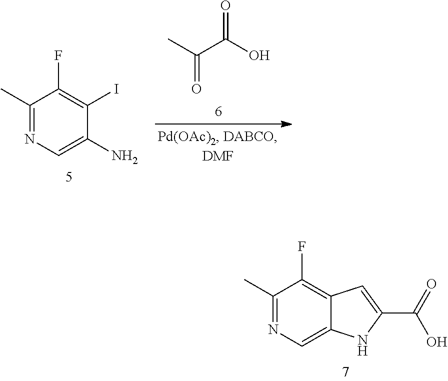

A mixture of 5-fluoro-4-iodo-6-methyl-pyridin-3-amine (956 mg, 3.79 mmol, 1 eq), 2-oxopropanoic acid (668.08 mg, 7.59 mmol, 534.46 uL, 2 eq) and DABCO (851.00 mg, 7.59 mmol, 834.31 uL, 2 eq) in DMF (10 mL) was degassed and purged with N₂ for 3 times, tPd(OAc)₂ (170.32 mg, 758.65 umol, 0.2 eq) was then added. The mixture was stirred at 110° C. for 4 hr under N₂ atmosphere. LC-MS indicated desired mass. The reaction mixture was filtered. The filtrate was concentrated under reduced pressure to remove DMF. The residue was diluted with toluene (30 mL). The resulting suspension was sonicated for 30 min. The supernatant was poured off. The residue was diluted with H₂O (10 mL), and then adjusted to pH to 3-4 with aqueous HCl (1 N). The solid was collected by filtration. Compound 4-fluoro-5-methyl-1H-pyrrolo[2,3-c]pyridine-2-carboxylic acid (695 mg, 3.58 mmol, 81.35% yield) was obtained as a brown solid.

LCMS (ESI) m/z: 195.1 [M+H]⁺; ¹H NMR (500 MHz, DMSO-d₆) δ=13.54 (br s, 1H), 12.51 (s, 1H), 8.57 (s, 1H), 7.09 (s, 1H), 2.48 (s, 3H).

### Synthesis of N-(1,1-dimethylsilepan-4-yl)-4-fluoro-5-methyl-1H-pyrrolo[2,3-c]pyridine-2-carboxamide

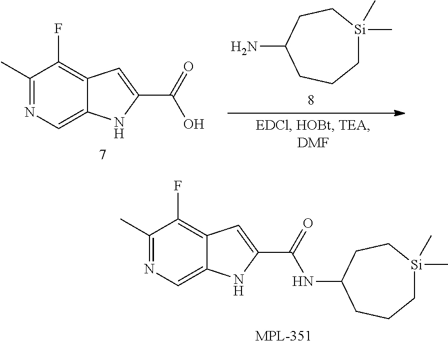

To a solution of 4-fluoro-5-methyl-1H-pyrrolo[2,3-c]pyridine-2-carboxylic acid (50 mg, 257.52 umol, 1 eq) and 1,1-dimethylsilepan-4-amine (59.88 mg, 309.02 umol, 1.2 eq, HCl salt) in DMF (1.5 mL) was added a solution of EDCI (148.10 mg, 772.55 umol, 3 eq) and HOBt (104.39 mg, 772.55 umol, 3 eq) in DMF (0.5 mL), followed by TEA (156.35 mg, 1.55 mmol, 215.06 uL, 6 eq). The mixture was stirred at 20° C. for 1 hr. LC-MS showed desired mass. The reaction mixture was filtered. The filtrate was purified by prep-HPLC (column: YMC-Actus Triart C18 150*30 mm*5 um; mobile phase: A: 0.05% formic acid in water, B: CH3CN, gradient: 30%-60% B over 11 min). Compound N-(1,1-dimethylsilepan-4-yl)-4-fluoro-5-methyl-1H-pyrrolo[2,3-c]pyridine-2-carboxamide (27.7 mg, 82.85 umol, 22.98% yield, 99.7% purity) was obtained as a white solid.

LCMS (ESI) m/z: 334.1 [M+H]⁺; ¹H NMR (500 MHz, METHANOL-d₄) 6=8.52 (s, 1H), 7.19 (d, J=0.6 Hz, 1H), 4.01-3.90 (m, 1H), 2.53 (d, J=3.2 Hz, 3H), 2.10-1.88 (m, 3H), 1.85-1.71 (m, 1H), 1.63-1.51 (m, 2H), 0.89-0.63 (m, 4H), 0.06 (d, J=8.9 Hz, 6H).

### Example 172. MPL-328

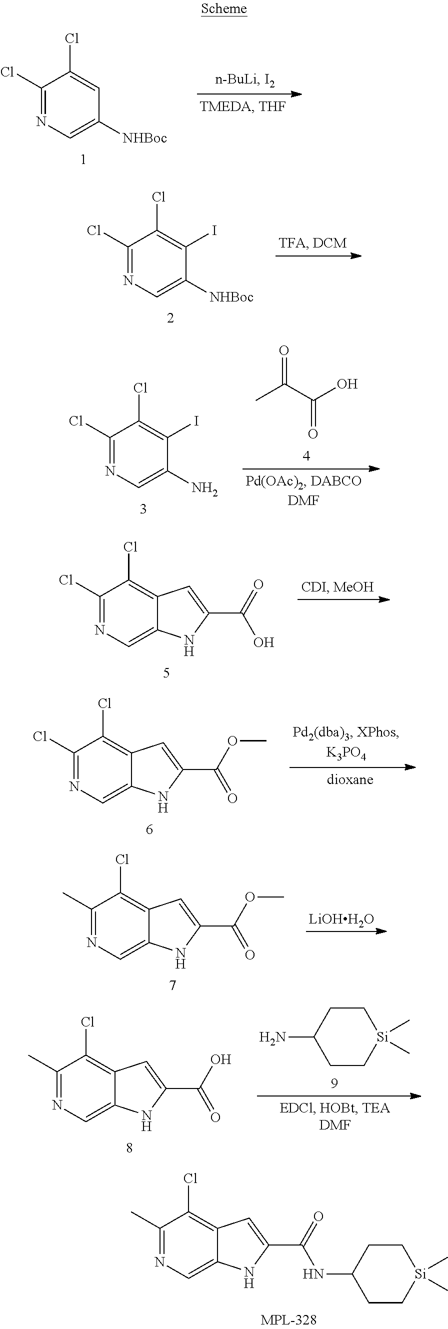

### Synthesis of tert-butyl N-(5,6-dichloro-4-iodo-3-pyridyl)carbamate

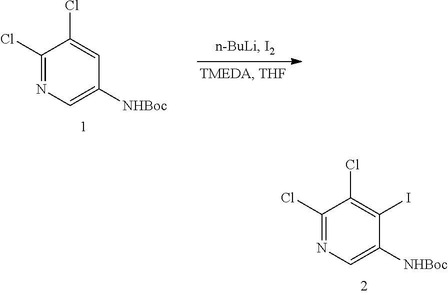 [SMILES: CC(C)(C)OC(=O)Nc1cnc(Cl)c(Cl)c1 ; CC(C)(C)OC(=O)Nc1cnc(Cl)c(Cl)c1I]

To a solution of tert-butyl N-(5,6-dichloro-3-pyridyl)carbamate (11.5 g, 43.71 mmol, 1 eq) and TMEDA (10.16 g, 87.41 mmol, 13.19 mL, 2 eq) in THF (10 mL) was added n-BuLi (2.5 M in n-hexane, 43.71 mL, 2.5 eq) dropwise at −78° C. under N₂. After stirring at −78° C. for 30 min, a solution of I₂ (16.64 g, 65.56 mmol, 13.21 mL, 1.5 eq) in THE (10 mL) was added dropwise at −78° C. The reaction mixture was stirred at −78° C. for another 30 min. TLC (Petroleum ether:Ethyl acetate=5:1) indicated compound 1 was consumed and many new spots formed. The reaction mixture was quenched with saturated Na₂SO₃ (60 mL) at 25° C., and then extracted with EtOAc (60 mL×3). The combined organic layer was washed with brine (50 mL), dried over Na₂SO₄, filtered and concentrated under reduced pressure. The resulting residue was purified by column chromatography (SiO₂, 0% to 6% ethyl acetate in petroleum ether). Compound tert-butyl N-(5,6-dichloro-4-iodo-3-pyridyl)carbamate (8.5 g, 18.57 mmol, 42.49% yield, 85% purity) was obtained as a white solid. ¹H NMR was recorded.

### Synthesis of 5,6-dichloro-4-iodo-pyridin-3-amine

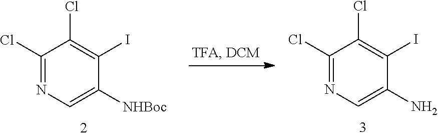 [SMILES: CC(C)(C)OC(=O)Nc1cnc(Cl)c(Cl)c1I ; Nc1cnc(Cl)c(Cl)c1I]

To a solution of tert-butyl N-(5,6-dichloro-4-iodo-3-pyridyl)carbamate (9.5 g, 24.42 mmol, 1 eq) in DCM (100 mL) was added TFA (162.56 g, 1.43 mol, 105.56 mL, 58.38 eq). The mixture was stirred at 30° C. for 12 hr. TLC (Petroleum ether:Ethyl acetate=5:1) indicated a new spot formed. The reaction mixture was concentrated under reduced pressure to remove solvent. The resulting residue was dissolved in saturated NaHCO₃ (100 mL), and then extracted with ethyl acetate (100 mL×2). The combined organic layer was washed with brine (50 mL), dried over Na₂SO₄, filtered and concentrated under reduced pressure. The residue was purified by column chromatography (SiO₂, 0-22% ethyl acetate in petroleum ether). Compound 5,6-dichloro-4-iodo-pyridin-3-amine (6.4 g, 21.05 mmol, 86.18% yield, 95% purity) was obtained as a white solid. ¹H NMR was recorded.

### Synthesis of 4,5-dichloro-1H-pyrrolo[2,3-c]pyridine-2-carboxylic acid

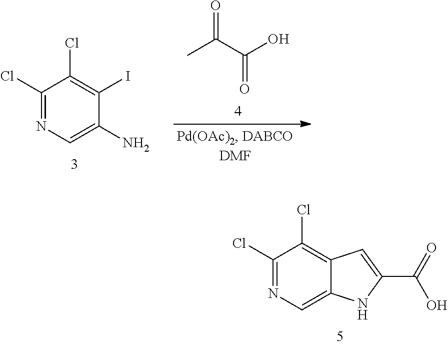

A mixture of 5,6-dichloro-4-iodo-pyridin-3-amine (3 g, 10.38 mmol, 1 eq), 2-oxopropanoic acid (1.83 g, 20.77 mmol, 1.46 mL, 2 eq), and DABCO (2.33 g, 20.77 mmol, 2.28 mL, 2 eq) in DMF (40 mL) was degassed and purged with N₂ for 3 times, and then Pd(OAc)₂ (466.27 mg, 2.08 mmol, 0.2 eq) was added into the solution. The mixture was stirred at 110° C. for 4 hr under N₂ atmosphere. LCMS showed desired mass. The reaction mixture was filtered. The filtrate was concentrated under reduced pressure to remove DMF. The residue was diluted with toluene (60 mL). The suspension was sonicated for 30 minutes. The supernatant was removed. The residue was diluted with H₂O (50 mL), and pH was adjusted to 3-4 using aqueous HCl (1 N) and then filtered to collect the solid. Compound 4,5-dichloro-1H-pyrrolo[2,3-c]pyridine-2-carboxylic acid (2.7 g, 9.35 mmol, 90.03% yield, 80% purity) was obtained as a brown solid. The crude product was used for the next step without further purification.

LCMS (ESI) m/z 231.0 [M+H]⁺; ¹H NMR was recorded.

### Synthesis of methyl 4,5-dichloro-1H-pyrrolo[2,3-c]pyridine-2-carboxylate

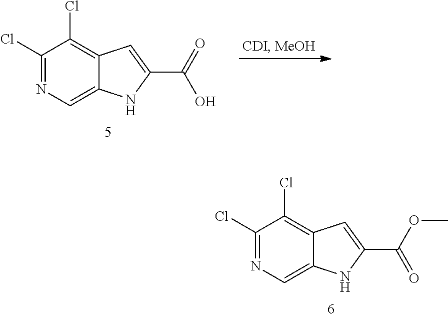

A mixture of 4,5-dichloro-1H-pyrrolo[2,3-c]pyridine-2-carboxylic acid (2.7 g, 11.69 mmol, 1 eq) and CDI (2.08 g, 12.86 mmol, 1.1 eq) in DMF (30 mL) was stirred at 30° C. for 1 hr. MeOH (23.75 g, 741.35 mmol, 30 mL, 63.44 eq) was then added. The mixture was stirred at 30° C. for 1 hr. LCMS indicated desired mass was detected. The reaction mixture was concentrated under reduced pressure to remove MeOH and then poured into H₂O (300 mL). The precipitates were collected by filtration and dried under reduced pressure. Compound methyl 4,5-dichloro-1H-pyrrolo[2,3-c]pyridine-2-carboxylate (480 mg, 1.86 mmol, 57.43% yield, 95% purity) was obtained as a brown solid, which was used for the next step without further purification.

LCMS (ESI) m/z: 245.0 [M+H]⁺

### Synthesis of methyl 4-chloro-5-methyl-1H-pyrrolo[2,3-c]pyridine-2-carboxylate

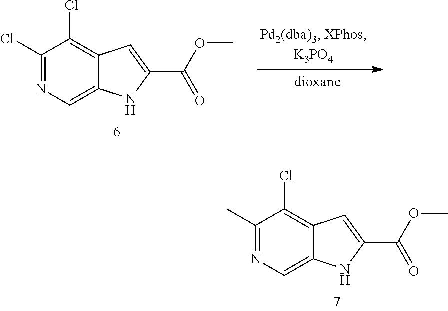

A mixture of methyl 4,5-dichloro-1H-pyrrolo[2,3-c]pyridine-2-carboxylate (500 mg, 2.04 mmol, 1 eq), methylboronic acid (610.66 mg, 10.20 mmol, 5 eq), K₃PO₄ (1.30 g, 6.12 mmol, 3 eq) and XPhos (194.53 mg, 408.06 umol, 0.2 eq) in dioxane (25 mL) was de-gassed under N₂ atmosphere. Pd₂(dba)₃ (373.67 mg, 408.06 umol, 0.2 eq) was then added. The suspension was degassed and purged with N₂ for 3 times. The mixture was stirred under N₂ at 120° C. for 12 hr. LC-MS showed desired mass was detected. EtOAc (60 mL) was added. The mixture was filtered to remove the insoluble materials. The filtrate was concentrated in vacuo. The resulting residue was purified by column chromatography (SiO₂, 0% to 34% ethyl acetate in petroleum ether). Compound methyl 4-chloro-5-methyl-1H-pyrrolo[2,3-c]pyridine-2-carboxylate (62 mg, crude) was obtained as a yellow solid.

LCMS (ESI) m/z: 225.1 [M+H]⁺; ¹H NMR was recorded.

### Synthesis of 4-chloro-5-methyl-1H-pyrrolo[2,3-c]pyridine-2-carboxylic acid

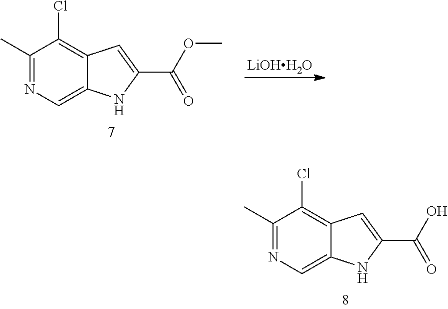

To a solution of methyl 4-chloro-5-methyl-1H-pyrrolo[2,3-c]pyridine-2-carboxylate (110 mg, 489.67 umol, 1 eq) in THE (2 mL) was added a solution of LiOH.H₂O (123.29 mg, 2.94 mmol, 6 eq) in H₂O (2 mL). The mixture was stirred at 60° C. for 2 hr. LC-MS showed desired mass was detected. The reaction mixture was concentrated under reduced pressure to remove THF. The aqueous phase was adjusted to pH 3-4 with aqueous HCl (6 N), and then purified by prep-HPLC (column: YMC-Actus Triart C18 150*30 mm*5 um; mobile phase: A: 0.225% formic acid in water, B: CH3CN; gradient: 30%-60% B over 11 min). Compound 4-chloro-5-methyl-1H-pyrrolo[2,3-c]pyridine-2-carboxylic acid (15 mg, crude) was obtained as a white solid.

LCMS (ESI) m/z: 211.0 [M+H]⁺; ¹H NMR (500 MHz, DMSO-d₆) δ=12.39 (s, 1H), 8.45 (s, 1H), 7.19 (s, 1H), 2.51 (s, 3H)

### Synthesis of 4-chloro-N-(1,1-dimethylsilinan-4-yl)-5-methyl-1H-pyrrolo[2,3-c]pyridine-2-carboxamide

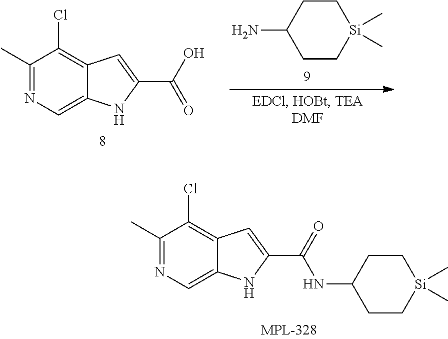

To a solution of 4-chloro-5-methyl-1H-pyrrolo[2,3-c]pyridine-2-carboxylic acid (15 mg, 71.22 umol, 1 eq) and 1,1-dimethylsilinan-4-amine (15.36 mg, 85.46 umol, 1.2 eq, HCl salt) in DMF (2 mL) was added a solution of EDCI (40.96 mg, 213.66 umol, 3 eq) and HOBt (28.87 mg, 213.66 umol, 3 eq) in DMF (0.5 mL), followed by TEA (43.24 mg, 427.32 umol, 59.48 uL, 6 eq). The mixture was stirred at 25° C. for 1 hr. LCMS showed desired mass was detected. The reaction mixture was filtered. The filterate was purified by prep-HPLC (column: YMC-Actus Triart C18 150*30 mm*5 um; mobile phase: A: 0.225% formic acid in water, B: CH₃CN; gradient: 60%-80% B over 11 min) Compound 4-chloro-N-(1,1-dimethylsilinan-4-yl)-5-methyl-1H-pyrrolo[2,3-c]pyridine-2-carboxamide (9.7 mg, 28.88 umol, 40.55% yield, 100% purity) was obtained as a white solid.

LCMS (ESI) m/z: 336.0 [M+H]⁺; ¹H NMR (500 MHz, DMSO-d₆) δ=10.86 (br s, 1H), 8.64 (s, 1H), 6.88 (s, 1H), 6.50 (br s, 1H), 4.03-3.84 (m, 1H), 2.59 (s, 3H), 2.28-2.14 (m, 2H), 1.72-1.56 (m, 2H), 0.86-0.80 (m, 2H), 0.77-0.64 (m, 2H), 0.09 (d, J=18.9 Hz, 6H).

### Example 173. MPL-319

### Synthesis of 4-fluoro-5-methyl-N-(5-silaspiro[4.5]decan-8-yl)-1H-pyrrolo[2,3-c]pyridine-2-carboxamide

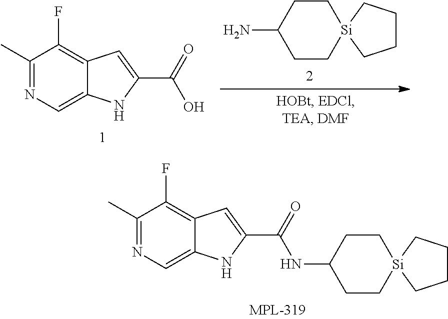 [SMILES: Cc1ncc2[nH]c(C(=O)O)cc2c1F ; NC1CC[Si]2(CCCC2)CC1 ; Cc1ncc2[nH]c(C(=O)NC3CC[Si]4(CCCC4)CC3)cc2c1F]

To a solution of 4-fluoro-5-methyl-1H-pyrrolo[2,3-c]pyridine-2-carboxylic acid (50 mg, 257.52 umol, 1 eq), 5-silaspiro[4.5]decan-8-amine (79.50 mg, 386.28 umol, 1.5 eq, HCl salt) in DMF (1 mL) was added HOBt (104.39 mg, 772.55 umol, 3 eq), EDCI (148.10 mg, 772.55 umol, 3 eq) and TEA (156.35 mg, 1.55 mmol, 215.06 uL, 6 eq). The mixture was stirred at 25° C. for 1 hr. LCMS showed desired compound. The reaction mixture was diluted with water 5 mL and extracted with EtOAc 20 mL (10 mL×2). The combined organic layer was washed with 5% LiCl (10 mL×2), dried over Na₂SO₄, filtered and concentrated under reduced pressure. The residue was purified by prep-HPLC (column: YMC-Actus Triart C18 150×30 mm×5 um; mobile phase: A: 0.225% formic acid in water, B: CH3CN; gradient: 37%-65% B over 11 min). Compound 4-fluoro-5-methyl-N-(5-silaspiro[4.5]decan-8-yl)-1H-pyrrolo[2,3-c]pyridine-2-carboxamide (43.9 mg, 125.81 umol, 48.85% yield, 99.010% purity) was obtained as a white solid.

LCMS (ESI) m/z: 346.2 [M+H]⁺; ¹H NMR (400 MHz, METHANOL-d4) 6=8.51 (d, J=0.8 Hz, 1H), 8.19 (br s, 1H), 7.18 (s, 1H), 3.83 (br t, J=11.3 Hz, 1H), 2.52 (d, J=3.1 Hz, 3H), 2.21 (br d, J=11.3 Hz, 2H), 1.72-1.58 (m, 6H), 0.88-0.82 (m, 4H), 0.68 (br t, J=6.8 Hz, 2H), 0.59 (br t, J=7.0 Hz, 2H).

### Example 174. MPL-320

### Synthesis of 4-fluoro-5-methyl-N-(6-silaspiro[5.5]undecan-3-yl)-1H-pyrrolo[2,3-c]pyridine-2-c arboxamide

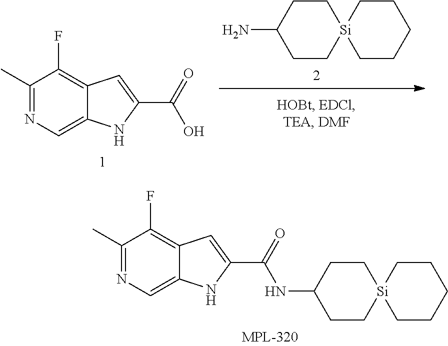 [SMILES: Cc1ncc2[nH]c(C(=O)O)cc2c1F ; NC1CC[Si]2(CCCCC2)CC1 ; Cc1ncc2[nH]c(C(=O)NC3CC[Si]4(CCCCC4)CC3)cc2c1F]

To a solution of 4-fluoro-5-methyl-1H-pyrrolo[2,3-c]pyridine-2-carboxylic acid (50 mg, 257.52 umol, 1 eq), 6-silaspiro[5.5]undecan-3-amine (67.93 mg, 309.02 umol, 1.2 eq, HCl salt) in DMF (1 mL) was added HOBt (104.39 mg, 772.55 umol, 3 eq), EDCI (148.10 mg, 772.55 umol, 3 eq) and TEA (156.35 mg, 1.55 mmol, 215.06 uL, 6 eq). The mixture was stirred at 25° C. for 1 hr. LCMS showed desired compound. The reaction mixture was diluted with CH₃OH (3 mL) and filtered. The filtrate was purified by prep-HPLC (column: YMC—Actus Triart C18 150×30 mm×5 um; mobile phase: A: 0.225% formic acid in water, B: CH₃CN; gradient 52%-77% B over 11 min). Compound 4-fluoro-5-methyl-N-(6-silaspiro[5.5]undecan-3-yl)-1H-pyrrolo[2,3-c]pyridine-2-carboxamide (55 mg, 152.99 umol, 59.41% yield, 100% purity) was obtained as a white solid.

LCMS (ESI) m/z: 360.2 [M+H]⁺; ¹H NMR (400 MHz, METHANOL-d4) 6=8.64 (s, 1H), 7.30 (s, 1H), 3.81 (br t, J=11.3 Hz, 1H), 2.60 (d, J=2.7 Hz, 3H), 2.15 (br d, J=9.8 Hz, 2H), 1.78-1.60 (m, 6H), 1.46 (br d, J=4.7 Hz, 2H), 0.98 (br d, J=14.9 Hz, 2H), 0.82-0.75 (m, 2H), 0.74-0.61 (m, 4H).

### Example 175. MPL-321

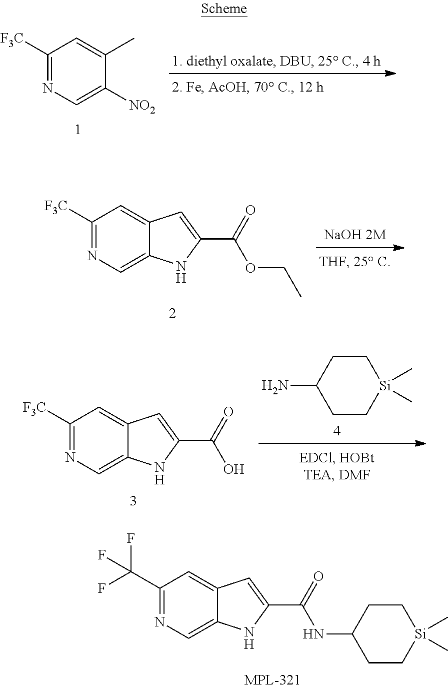

### Synthesis of ethyl 5-(trifluoromethyl)-1H-pyrrolo[2,3-c]pyridine-2-carboxylate

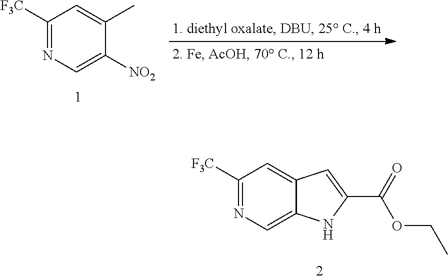 [SMILES: Cc1cc(C(F)(F)F)ncc1[N+](=O)[O-] ; CCOC(=O)c1cc2cc(C(F)(F)F)ncc2[nH]1]

To a solution of 4-methyl-5-nitro-2-(trifluoromethyl)pyridine (900 mg, 4.37 mmol, 1 eq) in diethyl oxalate (2.99 g, 20.48 mmol, 2.80 mL, 4.69 eq) was added DBU (1.58 g, 10.35 mmol, 1.56 mL, 2.37 eq). After stirring at 25° C. for 4 hr, the mixture was concentrated under reduced pressure. The residue was redissolved in AcOH (18.90 g, 314.73 mmol, 18.00 mL, 72.08 eq). The mixture was heated to 60° C. and Fe (487.68 mg, 8.73 mmol, 2 eq) was added. The mixture was stirred at 70° C. for 12 hr. TLC (Petroleum ether. EtOAc=3:1) showed one major spot. The mixture was poured into water (150 mL), filtered. The cake was re-dissolved in EtOAc (50 mL), washed with brine (50 mL), dried over Na₂SO₄, filtered and concentrated under reduced pressure. The resulting residue was purified by flash silica gel chromatography (0-30% Ethyl acetate in petroleum ether). Compound ethyl 5-(trifluoromethyl)-1H-pyrrolo[2,3-c]pyridine-2-carboxylate (800 mg, 2.94 mmol, 67.41% yield, 95% purity) was obtained as a light yellow solid. ¹H NMR was recorded.

### Synthesis of 5-(trifluoromethyl)-1H-pyrrolo[2,3-c]pyridine-2-carboxylic acid

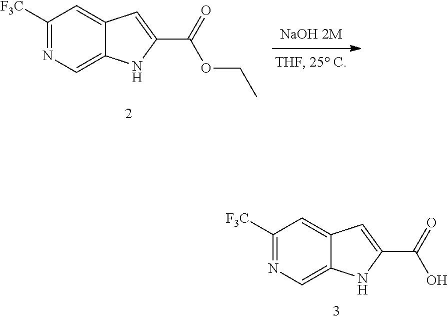 [SMILES: CCOC(=O)c1cc2cc(C(F)(F)F)ncc2[nH]1 ; O=C(O)c1cc2cc(C(F)(F)F)ncc2[nH]1]

To a solution of ethyl 5-(trifluoromethyl)-1H-pyrrolo[2,3-c]pyridine-2-carboxylate (800 mg, 3.10 mmol, 1 eq) in THE (10 mL) was added NaOH (1.60 g, 40.00 mmol, 12.91 eq) in H₂O (10 mL). The mixture was stirred at 25° C. for 60 hr. TLC (Petroleum ether:EtOAc=5:1) showed the starting material was remained, one new spot formed. The mixture was stirred at 40° C. for additional 12 hr. TLC (Petroleum ether:EtOAc=5:1) showed the starting material was consumed completely. The mixture was concentrated under reduced pressure to remove THF. Aq. HCl (3N) was added to adjust pH to 3. Solid was collected by filtration. The cake was washed by water (5 mL×2) and petroleum ether (5 mL×2) and dried by lyophilization. Compound 5-(trifluoromethyl)-1H-pyrrolo[2,3-c]pyridine-2-carboxylic acid (700 mg, 2.89 mmol, 93.26% yield, 95% purity) was obtained as a light yellow solid.

¹H NMR (500 MHz, DMSO-d₆) δ=13.72 (br s, 1H), 12.79 (br s, 1H), 8.92 (s, 1H), 8.21 (s, 1H), 7.29 (d, J=1.2 Hz, 1H)

### Synthesis of N-(1,1-dimethylsilinan-4-yl)-5-(trifluoromethyl)-1H-pyrrolo[2,3-c]pyridine-2-carboxamide

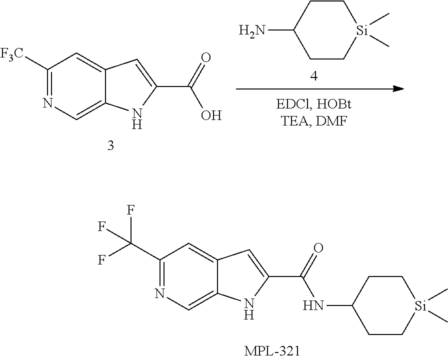

To a solution of 5-(trifluoromethyl)-1H-pyrrolo[2,3-c]pyridine-2-carboxylic acid (450 mg, 1.96 mmol, 1 eq) and 1,1-dimethylsilinan-4-amine (421.79 mg, 2.35 mmol, 1.2 eq, HCl) in DMF (5 mL) was added a solution of EDCI (749.67 mg, 3.91 mmol, 2 eq) and HOBt (528.41 mg, 3.91 mmol, 2 eq) in DMF (5 mL), followed by TEA (791.42 mg, 7.82 mmol, 1.09 mL, 4 eq). The mixture was stirred at 20° C. for 2 hr. LCMS showed reactant was consumed completely and one main peak with desired mass was detected. The mixture was poured into aqueous NaHCO₃ solution (1 g in 100 mL water), and then filtered. The filter cake was washed with water (30 mL) under ultrasound for 1 h, and then filtered. The solid was then dried in vacuo for 2 h. Compound N-(1,1-dimethylsilinan-4-yl)-5-(trifluoromethyl)-1H-pyrrolo[2,3-c]pyridine-2-carboxamide (512 mg, 1.44 mmol, 73.67% yield, 100% purity) was obtained as a white solid.

LCMS (ESI) m/z 356.1 [M+H]⁺; ¹H NMR (500 MHz, DMSO-d₆) δ=12.42 (br s, 1H), 8.78 (s, 1H), 8.52 (d, J=8.1 Hz, 1H), 8.12 (d, J=0.8 Hz, 1H), 7.27 (s, 1H), 3.69-3.59 (m, 1H), 1.96-1.85 (m, 2H), 1.58-1.46 (m, 2H), 0.69 (br d, J=14.5 Hz, 2H), 0.53 (dt, J=4.8, 14.2 Hz, 2H), 0.05-10 (m, 6H).

### Example 176. MPL-322

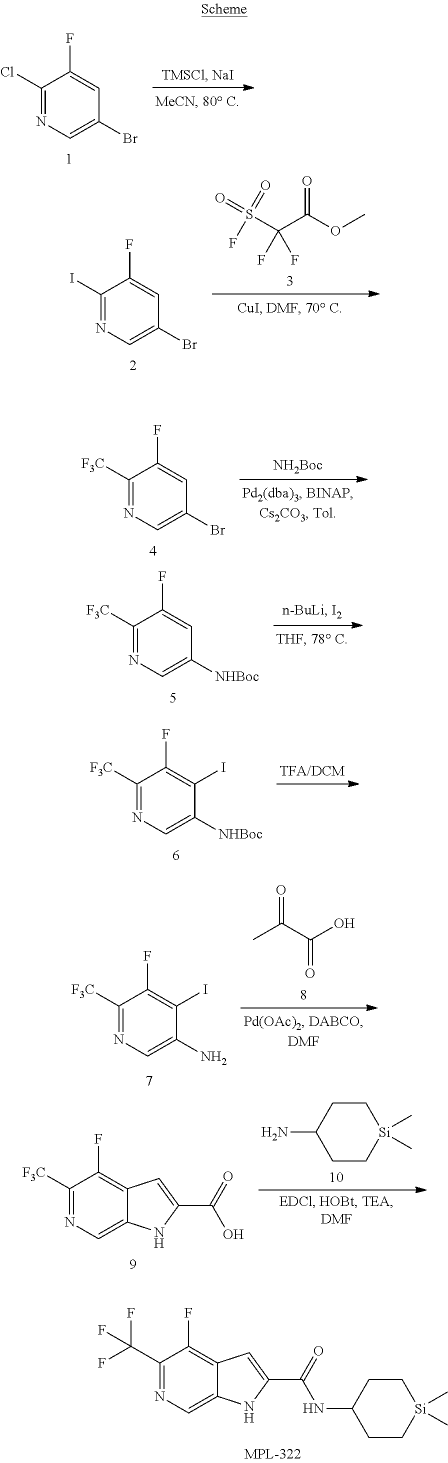

### Synthesis of 5-bromo-3-fluoro-2-iodo-pyridine

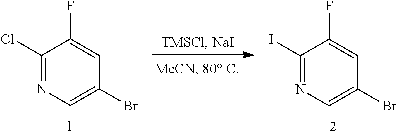 [SMILES: Fc1cc(Br)cnc1Cl ; Fc1cc(Br)cnc1I]

To a solution of 5-bromo-2-chloro-3-fluoro-pyridine (5 g, 23.76 mmol, 1 eq) and NaI (10.68 g, 71.28 mmol, 3 eq) in CH₃CN (20 mL) was added TMSCl (2.58 g, 23.76 mmol, 3.02 mL, 1 eq). The mixture was stirred at 80° C. for 2 hr under N₂. LC-MS showed reactant and desired mass. The reaction was stirred at 80° C. for additional 12 hr. TLC (Petroleum ether:EtOAc=20:1) showed one major new spot with higher polarity. The reaction was quenched with saturated Na₂SO₃ (50 mL), and then concentrated under reduced pressure to remove CH₃CN. The aqueous phase was extracted with EtOAc (20 mL×3). The combined organic layer was dried over Na₂SO₄, filtered and concentrated under reduced pressure. The resulting residue was purified by flash silica gel chromatography (0-3% ethyl acetate in petroleum ether). All fractions containing product (checked by TLC, Petroleum ether:EtOAc=20:1, Rf=0.5) were collected and concentrated. Compound 5-bromo-3-fluoro-2-iodo-pyridine (2.5 g, crude) was obtained as a yellow oil.

### Synthesis of 5-bromo-3-fluoro-2-(trifluoromethyl)pyridine

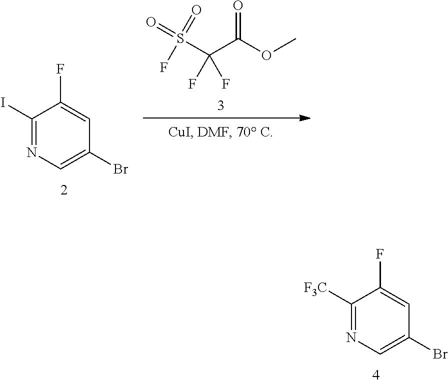

To a solution of 5-bromo-3-fluoro-2-iodo-pyridine (4 g, 6.63 mmol, 1 eq) and methyl 2,2-difluoro-2-fluorosulfonyl-acetate (8.91 g, 46.38 mmol, 5.90 mL, 7 eq) in DMF (140 mL) was added CuI (8.83 g, 46.38 mmol, 7 eq). The mixture was stirred under N₂ at 60° C. for 12 hr. LCMS showed reactant was consumed completely and desired mass was detected. The mixture was filtered. The cake was washed with EtOAc (10 mL×3). The combined filtrate was concentrated under reduced pressure. The residue was diluted with EtOAc (200 mL), washed with LiCl (3%, 100 mL×2) and brine (100 mL). The organic layer was dried over Na₂SO₄, filtered and concentrated. The resulting residue was purified by flash silica gel chromatography (0-5% Ethyl acetate in petroleum ether). Fractions containing desired product (checked by TLC. Petroleum ether:EtOAc=20:1) were combined and concentrated. Compound 5-bromo-3-fluoro-2-(trifluoromethyl)pyridine (900 mg, 1.84 mmol, 27.84% yield, 50% purity) was obtained as a yellow oil. ¹H NMR was recorded. It was used for the next step without further purification.

### Synthesis of tert-butyl N-[5-fluoro-6-(trifluoromethyl)-3-pyridyl]carbamate

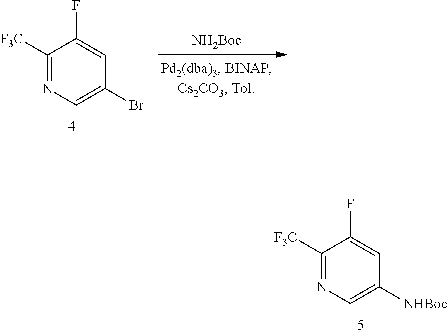 [SMILES: Fc1cc(Br)cnc1C(F)(F)F ; CC(C)(C)OC(=O)Nc1cnc(C(F)(F)F)c(F)c1 ; "NH2Boc"]

To a solution of 5-bromo-3-fluoro-2-(trifluoromethyl)pyridine (900 mg, 3.69 mmol, 1 eq), tert-butyl carbamate (1.30 g, 11.07 mmol, 3 eq) and Cs₂CO₃ (3.61 g, 11.07 mmol, 3 eq) in toluene (60 mL) was added Pd₂(dba)₃ (675.57 mg, 737.75 umol, 0.2 eq) and BINAP (689.07 mg, 1.11 mmol, 0.3 eq) under N₂. The mixture was degassed with N₂ for 15 min, and stirred and refluxed at 110° C. for 12 hr. LCMS showed reactant was consumed completely and desired mass was detected. The mixture was filtered. The cake was washed with EtOAc (20 mL×3). The combined filtrate was concentrated under reduced pressure. The resulting residue was purified by flash silica gel chromatography (0-8% ethyl acetate in petroleum ether). The fractions containing desired product (Checked by TLC, Petroleum ether:EtOAc=5:1) were collected and concentrated. The resulting residue was further purified by prep-HPLC (column: YMC-Actus Triart C18 150*30 mm*5 um; mobile phase: A: 0.225% formic acid in water, B: CH3CN; gradient: 52%-82% B over 11 min). Compound tert-butyl N-[5-fluoro-6-(trifluoromethyl)-3-pyridyl]carbamate (240 mg, 813.65 umol, 22.06% yield, 95% purity) was obtained as a white solid. ¹H NMR was recorded.

### Synthesis of tert-butyl N-[5-fluoro-4-iodo-6-(trifluoromethyl)-3-pyridyl]carbamate

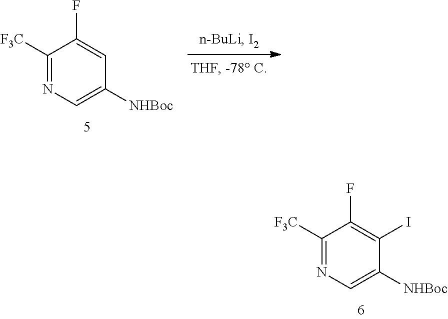 [SMILES: CC(C)(C)OC(=O)Nc1cnc(C(F)(F)F)c(F)c1 ; CC(C)(C)OC(=O)Nc1cnc(C(F)(F)F)c(F)c1I]

A solution of tert-butyl N-[5-fluoro-6-(trifluoromethyl)-3-pyridyl]carbamate (230 mg, 820.79 umol, 1 eq) in THF (10 mL) was purged with N₂. TMEDA (286.14 mg, 2.46 mmol, 371.61 uL, 3 eq) was then added. The mixture was cooled to −75° C. and n-BuLi (2.5 M in n-hexane, 820.79 uL, 2.5 eq) was added dropwise to maintain temperature below −70° C. After addition, the mixture was stirred at −75° C.˜−70° C. for 3 hr. Then a solution of I₂ (312.48 mg, 1.23 mmol, 248.00 uL, 1.5 eq) in THF (2 mL) was added at −70° C. dropwise to maintain temperature below −70° C. The mixture was stirred at −75° C.˜−70° C. for 2 hr. TLC (Petroleum ether:EtOAc=5:1) showed starting material was consumed completely and one major new sport formed. The reaction was quenched with saturated Na₂SO₃ (20 mL). The mixture was concentrated under reduced pressure to remove THF, and then extracted with EtOAc (10 mL×3). The combined organic layer was washed with brine (20 mL), dried over Na₂SO₄, filtered and concentrated. The resulting residue was purified by flash silica gel chromatography (0-20% Ethyl acetate in petroleum ether). Compound tert-butyl N-[5-fluoro-4-iodo-6-(trifluoromethyl)-3-pyridyl]carbamate (240 mg, 561.42 umol, 68.40% yield, 95% purity) was obtained as a white solid. ¹H NMR was recorded.

### Synthesis of 5-fluoro-4-iodo-6-(trifluoromethyl)pyridin-3-amine

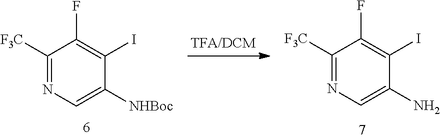 [SMILES: CC(C)(C)OC(=O)Nc1cnc(C(F)(F)F)c(F)c1I ; Nc1cnc(C(F)(F)F)c(F)c1I]

To a solution of tert-butyl N-[5-fluoro-4-iodo-6-(trifluoromethyl)-3-pyridyl]carbamate (240 mg, 590.97 umol, 1 eq) in DCM (2.5 mL) was added TFA (11.55 g, 101.30 mmol, 7.5 mL, 171.41 eq). The mixture was stirred at 25° C. for 3 hr. TLC (Petroleum ether:EtOAc=3:1) showed starting material was consumed completely and one new spot formed. Saturated NaHCO₃ was added to adjust pH to 8. The product was extracted with EtOAc (15 mL×2). The combined organic layer was dried over Na₂SO₄, filtered and concentrated. Compound 5-fluoro-4-iodo-6-(trifluoromethyl)pyridin-3-amine (190 mg, 589.87 umol, 99.81% yield, 95% purity) was obtained as a light yellow solid. ¹H NMR was recorded.

### Synthesis of 4-fluoro-5-(trifluoromethyl)-1H-pyrrolo[2,3-c]pyridine-2-carboxylic acid

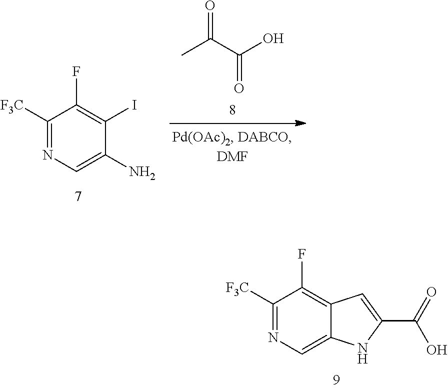

To a mixture of 5-fluoro-4-iodo-6-(trifluoromethyl)pyridin-3-amine (169 mg, 552.29 umol, 1 eq), 2-oxopropanoic acid (116.73 mg, 1.33 mmol, 93.38 uL, 2.4 eq) and DABCO (123.90 mg, 1.10 mmol, 121.47 uL, 2 eq) was added DMF (8 mL). Then Pd(OAc)₂ (62.00 mg, 276.14 umol, 0.5 eq) was added under N₂. The mixture was purged with N₂ for 15 min then stirred at 115° C. for 5 h. LCMS showed reactant was consumed completely and one main peak with desired mass was detected. The mixture was concentrated under reduced pressure to remove DMF. The residue was redissolved in MeOH and filtered to remove insoluble matter. The filtrate was purified by prep-HPLC (column: YMC-Actus Triart C18 150*30 mm*5 um; mobile phase: A: 0.225% formic acid in water, B: CH₃CN; gradient 6%-60% B over 11 min). Compound 4-fluoro-5-(trifluoromethyl)-1H-pyrrolo[2,3-c]pyridine-2-carboxylic acid (85 mg, 325.43 umol, 58.92% yield, 95% purity) was obtained as a white solid.

LCMS (ESI) m/z 248.9 [M+H]⁺

### Synthesis of N-(1,1-dimethylsilinan-4-yl)-4-fluoro-5-(trifluoromethyl)-1H-pyrrolo[2,3-c]pyridine-2-carboxamide

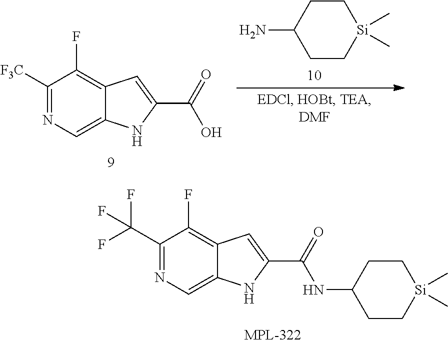

To a solution of 4-fluoro-5-(trifluoromethyl)-1H-pyrrolo[2,3-c]pyridine-2-carboxylic acid (65 mg, 261.96 umol, 1 eq) and 1,1-dimethylsilinan-4-amine (61.22 mg, 340.54 umol, 1.3 eq, HCl salt) in DMF (0.7 mL) was added a solution of EDCI (100.43 mg, 523.91 umol, 2 eq) and HOBt (70.79 mg, 523.91 umol, 2 eq) in DMF (0.8 mL), followed by TEA (106.03 mg, 1.05 mmol, 145.84 uL, 4 eq). The mixture was stirred at 25° C. for 2 hr. LCMS showed reactant was consumed completely and one main peak with desired mass was detected. The mixture was filtered to remove insoluble matter. The filtrate was purified by prep-HPLC (column: YMC-Actus Triart C18 150*30 mm*5 um; mobile phase: A: 0.225% formic acid in water, B: CH₃CN, gradient 66%-92% B over 11 min). Compound N-(1,1-dimethylsilinan-4-yl)-4-fluoro-5-(trifluoromethyl)-1H-pyrrolo[2,3-c]pyridine-2-carboxamide (70 mg, 187.46 umol, 71.56% yield, 100% purity) was obtained as a white solid.

LCMS (ESI) m/z 374.1 [M+H]⁺; ¹H NMR (500 MHz, DMSO-d₆) δ=13.45-12.02 (m, 1H), 8.61 (d, J=1.7 Hz, 1H), 8.52 (br d, J=8.1 Hz, 1H), 7.43 (s, 1H), 3.72-3.59 (m, 1H), 1.98-1.87 (m, 2H), 1.58-1.47 (m, 2H), 0.69 (br d, J=14.5 Hz, 2H), 0.53 (dt, J=4.7, 14.1 Hz, 2H), 0.00 (s, 3H), −0.06 (s, 3H).

### Example 177. MPL-329

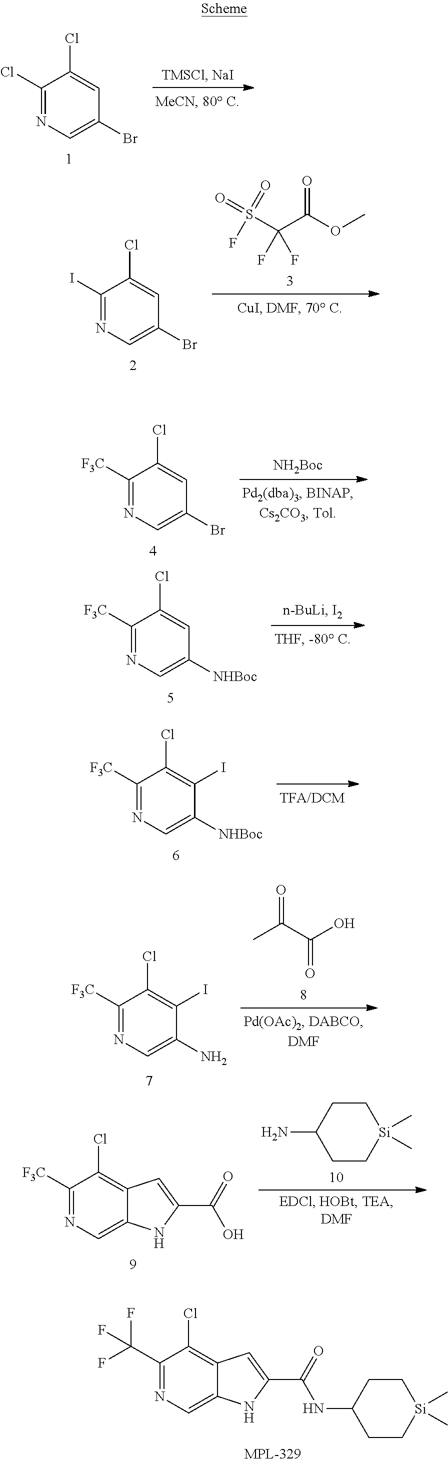

### Synthesis of 5-bromo-3-chloro-2-iodo-pyridine

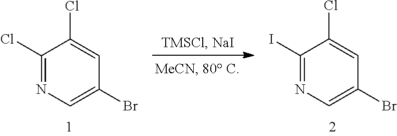 [SMILES: Clc1cc(Br)cnc1Cl ; Clc1cc(Br)cnc1I]

To a solution of 5-bromo-2,3-dichloro-pyridine (3 g, 13.22 mmol, 1 eq) and NaI (5.95 g, 39.67 mmol, 3 eq) in CH₃CN (30 mL) was added TMSCl (1.44 g, 13.22 mmol, 1.68 mL, 1 eq). The mixture was stirred at 80° C. under N₂ for 2 hr. LCMS showed one main peak with desired mass but compound 1 was also detected. The reaction was stirred at 80° C. for additional 12 hr. LCMS showed desired mass and compound 1 was almost consumed completely. The reaction was quenched with saturated Na₂SO₃ (60 mL). The mixture was concentrated under reduced pressure to remove CH₃CN, and then was extracted with EtOAc (20 mL×3). The combined organic layer was washed with brine (40 mL×2), dried over Na₂SO₄, filtered and concentrated. The resulting residue was purified by flash silica gel chromatography (0-2% Ethyl acetate in petroleum ether). The fractions containing product (checked by TLC. Petroleum ether:EtOAc=20:1) were combined and concentrated. Compound 5-bromo-3-chloro-2-iodo-pyridine (3 g, 7.54 mmol, 57.02% yield, 80% purity) was obtained as a white solid. It was used for the next step without further purification.

### Synthesis of 5-bromo-3-chloro-2-(trifluoromethyl)pyridine

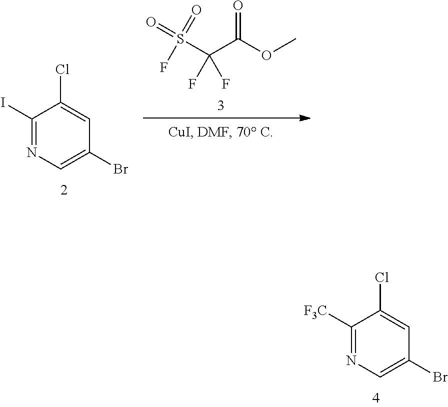

To a solution of 5-bromo-3-chloro-2-iodo-pyridine (3 g, 4.71 mmol, 1 eq) and methyl 2,2-difluoro-2-fluorosulfonyl-acetate (6.34 g, 32.98 mmol, 4.20 mL, 7 eq) in DMF (100 mL) was added CuI (6.28 g, 32.98 mmol, 7 eq). The mixture was stirred under N₂ at 60° C. for 12 hr. LC-MS showed reactant 2 was consumed completely and desired mass was detected. The mixture was concentrated under reduced pressure. The product was found in the residue as well as in the solution collected in flask (checked by TLC; petroleum ether:EtOAc=20:1). The solution was poured to water (800 mL) and extracted with a mixture of petroleum ether and EtOAc (10:1, 300 mL). The organic layer was dried over Na₂SO₄, filtered and concentrated under reduced pressure to give compound 5-bromo-3-chloro-2-(trifluoromethyl)pyridine (890 mg, 2.73 mmol, 58.02% yield, 80% purity) as a colorless oil. ¹H NMR was recorded.

### Synthesis of tert-butyl N-[5-chloro-6-(trifluoromethyl)-3-pyridyl]carbamate

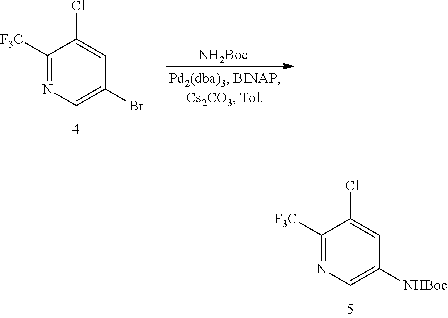 [SMILES: FC(F)(F)c1ncc(Br)cc1Cl ; CC(C)(C)OC(=O)Nc1cnc(C(F)(F)F)c(Cl)c1 ; "NH2Boc"]

To a mixture of 5-bromo-3-chloro-2-(trifluoromethyl)pyridine (600 mg, 2.30 mmol, 1 eq), tert-butyl carbamate (809.64 mg, 6.91 mmol, 3 eq) and Cs₂CO₃ (2.25 g, 6.91 mmol, 3 eq) in toluene (45 mL) was added Pd₂(dba)₃ (421.93 mg, 460.76 umol, 0.2 eq) and BINAP (430.35 mg, 691.14 umol, 0.3 eq) under N₂. The mixture was degassed with N₂ for 15 min and then stirred and refluxed at 110° C. for 12 hr. LC-MS showed reactant 4 was consumed completely and desired mass was detected. The mixture was filtered. The cake was washed with EtOAc (20 mL×3). The combined filtrate was concentrated under reduced pressure. The resulting residue was purified by flash silica gel chromatography (0-12% Ethyl acetate in petroleum ether). The fractions containing product (checked by TLC, petroleum ether:EtOAc=8:1) were collected and concentrated. Compound tert-butyl N-[5-chloro-6-(trifluoromethyl)-3-pyridyl]carbamate (360 mg, 1.15 mmol, 50.04% yield, 95% purity) was obtained as a yellow solid. ¹H NMR was recorded.

### Synthesis of tert-butyl N-[5-fluoro-4-iodo-6-(trifluoromethyl)-3-pyridyl]carbamate

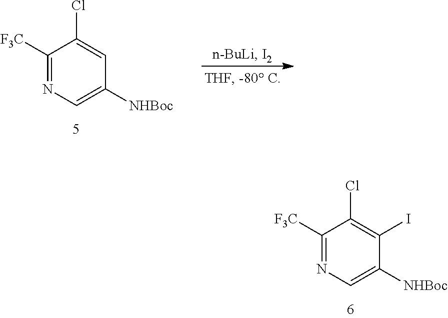

To a solution of tert-butyl N-[5-chloro-6-(trifluoromethyl)-3-pyridyl]carbamate (480 mg, 1.62 mmol, 1 eq) in THF (20 mL) (dried by Na and distilled) was purged with N₂, and TMEDA (564.06 mg, 4.85 mmol, 732.55 uL, 3 eq) was then added. The mixture was cooled to −80° C., n-BuLi (2.5 M in n-hexane, 1.75 mL, 2.7 eq) was the added dropwise to maintain temperature below −80° C. After addition, the mixture was stirred at −80° C.˜−90° C. for 3 hr. Then a solution of I₂ (698.10 mg, 2.75 mmol, 554.05 uL, 1.7 eq) in THF (5 mL) was added at −80° C. dropwise to maintain temperature below −80° C. The mixture was stirred at −80° C.˜−90° C. for 2 hr. TLC (petroleum ether:EtOAc=5:1) showed one new sport formed. The reaction was quenched with saturated Na₂SO₃ (40 mL), concentrated under reduced pressure to remove THF. The aqueous solution was extracted with EtOAc (20 mL×3). The combined organic layer was washed with brine (40 mL), dried over Na₂SO₄, filtered and concentrated. The resulting residue was purified by flash silica gel chromatography (0-20% Ethyl acetate in petroleum ether). Compound tert-butyl N-[5-chloro-4-iodo-6-(trifluoromethyl)-3-pyridyl]carbamate (400 mg, 899.26 umol, 55.58% yield, 95% purity) was obtained as a light yellow solid. ¹H NMR was recorded.

### Synthesis of 5-chloro-4-iodo-6-(trifluoromethyl)pyridin-3-amine

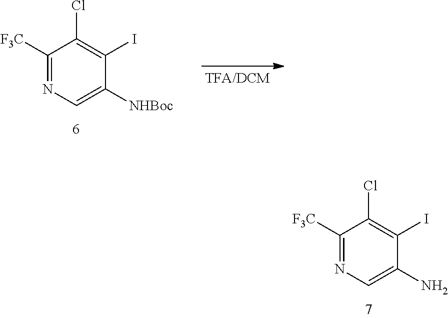 [SMILES: CC(C)(C)OC(=O)Nc1cnc(C(F)(F)F)c(Cl)c1I ; Nc1cnc(C(F)(F)F)c(Cl)c1I]

To a solution of tert-butyl N-[5-chloro-4-iodo-6-(trifluoromethyl)-3-pyridyl]carbamate (400 mg, 946.59 umol, 1 eq) in DCM (5 mL) was added TFA (23.10 g, 202.60 mmol, 15 mL, 214.03 eq). The mixture was stirred at 25° C. for 3 hr. TLC (Petroleum ether:EtOAc=3:1) showed starting material was consumed completely and one new spot formed. The mixture was concentrated under reduced pressure. The residue was poured into saturated NaHCO₃ (50 mL), and then extracted with EtOAc (20 mL). The organic layer was washed with brine (30 mL), dried over Na₂SO₄, filtered and concentrated. Compound 5-chloro-4-iodo-6-(trifluoromethyl)pyridin-3-amine (220 mg, 648.16 umol, 68.47% yield, 95% purity) was obtained as a light yellow solid. 1H NMR was recorded.

### Synthesis of 4-chloro-5-(trifluoromethyl)-1H-pyrrolo[2,3-c]pyridine-2-carboxylic acid

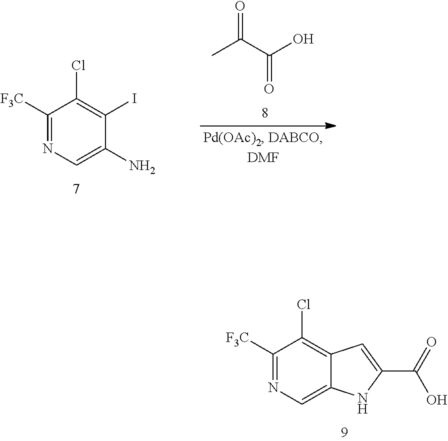

To a mixture of 5-chloro-4-iodo-6-(trifluoromethyl)pyridin-3-amine (200 mg, 620.24 umol, 1 eq), 2-oxopropanoic acid (81.93 mg, 930.37 umol, 65.54 uL, 1.5 eq) and DABCO (139.15 mg, 1.24 mmol, 136.42 uL, 2 eq) was added DMF (10 mL). Then Pd(OAc)₂ (55.70 mg, 248.10 umol, 0.4 eq) was added under N₂. The mixture was stirred at 115° C. for 4 hr. LCMS showed reactant 7 was consumed completely and one main peak with desired mass was detected. The mixture was concentrated under reduced pressure to remove DMF. The residue was redissolved in MeOH (4 mL), and then filtered to remove insoluble materials. The filtrate was purified by prep-HPLC (column: YMC-Actus Triart C18 150*30 mm*5 um; mobile phase: A: 0.225% formic acid in water, B: CH₃CN; gradient 38%-60% B over 1 min). Compound 4-chloro-5-(trifluoromethyl)-1H-pyrrolo[2,3-c]pyridine-2-carboxylic acid (28 mg, 105.82 umol, 17.06% yield) was obtained as a white solid.

LCMS (ESI) m/z 264.9 [M+H]⁺

### Synthesis of 4-chloro-N-(1,1-dimethylsilinan-4-yl)-5-(trifluoromethyl)-1H-pyrrolo[2,3-c]pyridine-2-carboxamide

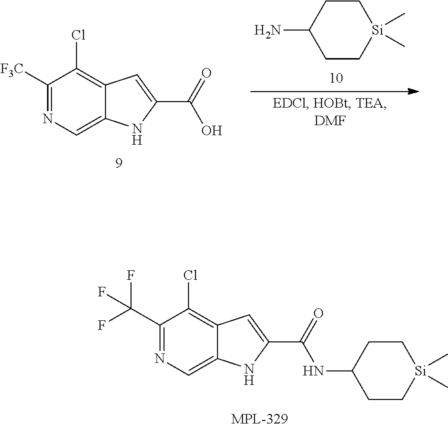

To a solution of 4-chloro-5-(trifluoromethyl)-1H-pyrrolo[2,3-c]pyridine-2-carboxylic acid (28 mg, 105.82 umol, 1 eq) and 1,1-dimethylsilinan-4-amine (26.63 mg, 148.15 umol, 1.4 eq, HCl salt) in DMF (1 mL) was added a solution of EDCI (40.57 mg, 211.65 umol, 2 eq) and HOBt (28.60 mg, 211.65 umol, 2 eq) in DMF (1 mL), followed by TEA (42.83 mg, 423.30 umol, 58.92 uL, 4 eq). The mixture was stirred at 25° C. for 2 hr. LCMS showed reactant remained. The mixture was stirred for additional 12 hr. LCMS showed reactant was consumed completely and one peak with desired mass was detected. The reaction mixture was filtered to remove insoluble matter. The filtrate was purified by prep-HPLC (column: YMC-Actus Triart C18 150*30 mm*5 um; mobile phase: A: 0.225% formic acid in water, B: CH₃CN; gradient: 50%-70% B over 11 min). Compound 4-chloro-N-(1,1-dimethylsilinan-4-yl)-5-(trifluoromethyl)-1H-pyrrolo[2,3-c]pyridine-2-carboxamide (27 mg, 69.25 umol, 65.44% yield, 100% purity) was obtained as a white solid.

LCMS (ESI) m/z 390.1 [M+H]⁺; ¹H NMR (500 MHz, DMSO-d₆) δ=12.79 (br s, 1H), 8.69 (s, 1H), 8.63 (br d, J=8.1 Hz, 1H), 8.66-8.59 (m, 1H), 7.45 (s, 1H), 3.65 (dt, J=8.0, 11.2 Hz, 1H), 1.92 (br d, J=9.5 Hz, 2H), 1.60-1.44 (m, 2H), 0.69 (br d, J=14.3 Hz, 2H), 0.53 (dt, J=4.8, 14.2 Hz, 2H), 0.04-−0.10 (m, 7H)

### Example 178. MPL-345

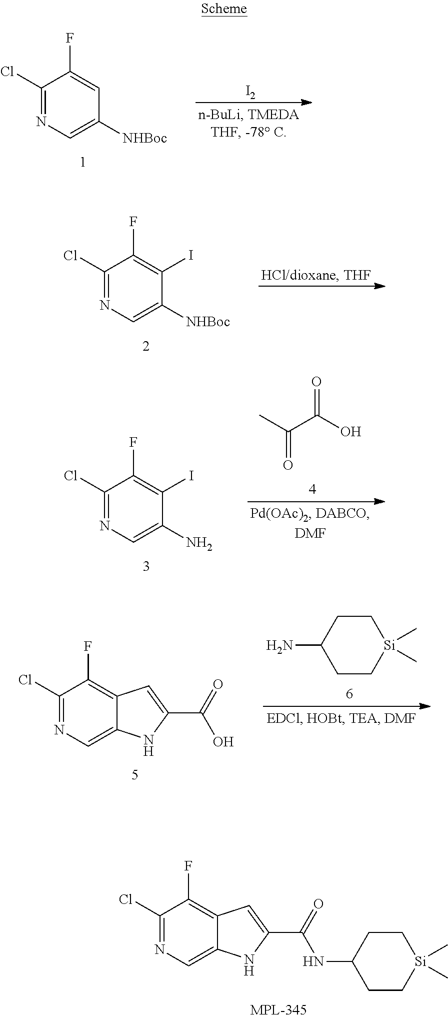

### Synthesis of tert-butyl N-(6-chloro-5-fluoro-4-iodo-3-pyridyl)carbamate

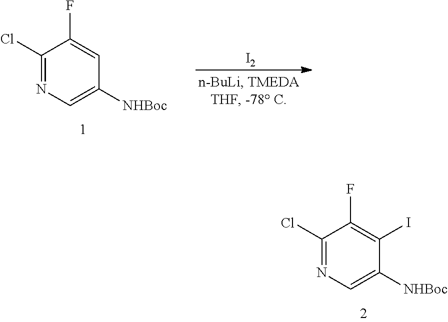 [SMILES: CC(C)(C)OC(=O)Nc1cnc(Cl)c(F)c1 ; "I2" ; CC(C)(C)OC(=O)Nc1cnc(Cl)c(F)c1I]

To a solution of tert-butyl N-(6-chloro-5-fluoro-3-pyridyl)carbamate (1 g, 4.05 mmol, 1 eq) and TMEDA (942.21 mg, 8.11 mmol, 1.22 mL, 2 eq) in THF (12 mL) was added n-BuLi (2.5 M in n-hexane, 4.05 mL, 2.5 eq) dropwise at −78° C. under N₂. The reaction mixture was stirred at −78° C. for 30 mins. A solution of I₂ (1.54 g, 6.08 mmol, 1.22 mL, 1.5 eq) in THF (5 mL) was added dropwise at −78° C. The reaction mixture was stirred at −78° C. for another 30 min. TLC (Petroleum ether:Ethyl acetate=3:1) indicated starting material was consumed completely and one new spot formed. The reaction mixture was quenched with saturated Na₂SO₃ (20 mL) at 25° C., and then diluted with H₂O (10 mL) and extracted with EtOAc (30 mL×2). The combined organic layer was washed with brine (20 mL), dried over Na₂SO₄, filtered and concentrated under reduced pressure. Compound tert-butyl N-(6-chloro-5-fluoro-4-iodo-3-pyridyl)carbamate (1.4 g, 3.57 mmol, 88.06% yield, 95% purity) was obtained as a white solid. ¹H NMR was recorded.

### Synthesis of 6-chloro-5-fluoro-4-iodo-pyridin-3-amine

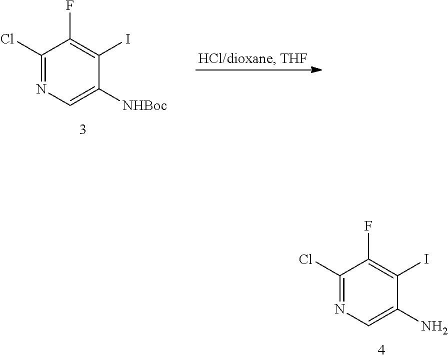 [SMILES: CC(C)(C)OC(=O)Nc1cnc(Cl)c(F)c1I ; "HCl/dioxane, THF" ; Nc1cnc(Cl)c(F)c1I]

To a solution of tert-butyl N-(6-chloro-5-fluoro-4-iodo-3-pyridyl)carbamate (1.4 g, 3.76 mmol, 1 eq) in THE (2 mL) was added HCl/dioxane (4 M, 5 mL, 5.32 eq). The mixture was stirred at 25° C. for 12 hr. TLC (Petroleum ether:Ethyl acetate=3:1) indicated starting material was consumed and one new spot formed. The resulting product was dissolved in Petroleum ether:Ethyl acetate=5:1 (30 mL) and filtered to remove insoluble materials. The filtrate was concentrated in vacuo. The resulting residue was dissolved in saturated NaHCO₃ (5 mL), the and extracted with EtOAc (15 mL×2). The combined organic layer was washed with brine (15 mL), dried over Na₂SO₄, filtered and concentrated under reduced pressure. Compound 6-chloro-5-fluoro-4-iodo-pyridin-3-amine (556 mg, 2.04 mmol, 54.31% yield) was obtained as a white solid. ¹H NMR was recorded.

### Synthesis of 5-chloro-4-fluoro-1H-pyrrolo[2,3-c]pyridine-2-carboxylic acid

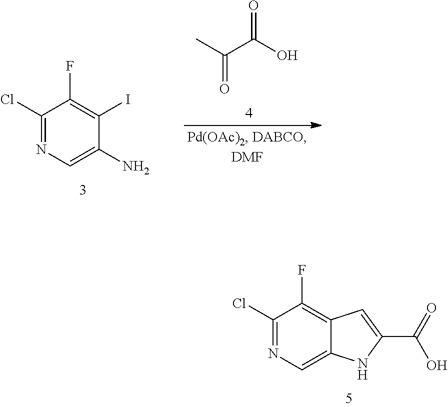

A mixture of 6-chloro-5-fluoro-4-iodo-pyridin-3-amine (456 mg, 1.67 mmol, 1 eq), 2-oxopropanoic acid (294.78 mg, 3.35 mmol, 235.83 uL, 2 eq), and DABCO (375.49 mg, 3.35 mmol, 368.13 uL, 2 eq) in DMF (5 mL) was degassed and purged with N₂ for 3 times, and then Pd(OAc)₂ (75.15 mg, 334.75 umol, 0.2 eq) was added. The mixture was stirred at 110° C. for 4 hr under N₂ atmosphere. LCMS showed desired mass. The reaction mixture was concentrated under reduced pressure to remove DMF. The residue was diluted with toluene (15 mL). The suspension was sonicated for 30 minutes, and the supernatant was poured off. The residue was diluted with H₂O (15 mL), and then adjusted to pH to 3-4 with aqueous HCl (1 N), and then filtered. The cake was collected and diluted with CH₃CN (6 mL). The suspension was sonicated for 10 minutes and filtered to collect solid. Compound 5-chloro-4-fluoro-1H-pyrrolo[2,3-c]pyridine-2-carboxylic acid (544 mg, crude) was obtained as a brown solid, which was used for the next step without further purification.

LCMS (ESI) m/z: 215.1 [M+H]⁺; ¹H NMR (400 MHz, DMSO-d₆) δ=12.81 (s, 1H), 8.48 (s, 1H), 7.17 (s, 1H).

### Synthesis of 5-chloro-N-(1,1-dimethylsilinan-4-yl)-4-fluoro-1H-pyrrolo[2,3-c]pyridine-2-carboxamide

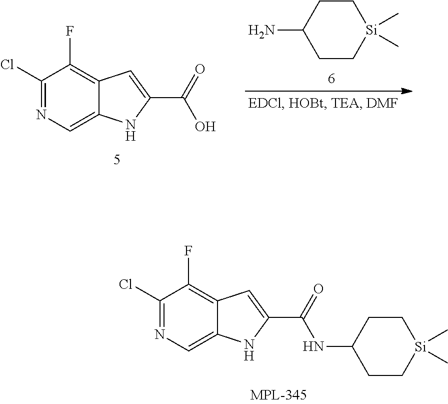

To a solution of 5-chloro-4-fluoro-1H-pyrrolo[2,3-c]pyridine-2-carboxylic acid (100 mg, 466.02 umol, 1 eq) and 1,1-dimethylsilinan-4-amine (100.53 mg, 559.23 umol, 1.2 eq, HCl salt) in DMF (2.5 mL) was added a solution of EDCI (268.01 mg, 1.40 mmol, 3 eq) and HOBt (188.91 mg, 1.40 mmol, 3 eq) in DMF (0.5 mL), followed by TEA (282.94 mg, 2.80 mmol, 389.19 uL, 6 eq) The mixture was stirred at 20° C. for 1 hr. LCMS showed desired mass. The reaction mixture was filtered to obtain the filter cake. The residue was purified by prep-HPLC (column: YMC-Actus Triart C18 150*30 mm*5 um; mobile phase: A: 0.225% formic acid in water, B: CH₃CN; gradient: 55%-85% B over 11 min). Compound 5-chloro-N-(1,1-dimethylsilinan-4-yl)-4-fluoro-1H-pyrrolo[2,3-c]pyridine-2-carboxamide (66.6 mg, 195.96 umol, 42.05% yield, 100% purity) was obtained as a pale-orange solid.

LCMS (ESI) m/z: 340.1 [M+H]⁺; ¹H NMR (500 MHz, DMSO-d₆) δ=12.61 (br s, 1H) 8.59 (d, J=8.09 Hz, 1H) 8.45 (s, 1H) 7.37 (s, 1H) 3.69-3.77 (m, 1H) 1.96-2.04 (m, 2H) 1.55-1.65 (m, 2H) 0.78 (br d, J=14.34 Hz, 2H) 0.62 (td, J=14.11, 4.58 Hz, 2H) 0.00-0.12 (m, 6H).

### Example 179. MPL-346

### Synthesis of 5-chloro-N-(1,1-dimethylsilepan-4-yl)-4-fluoro-1H-pyrrolo[2,3-c]pyridine-2-carboxamide

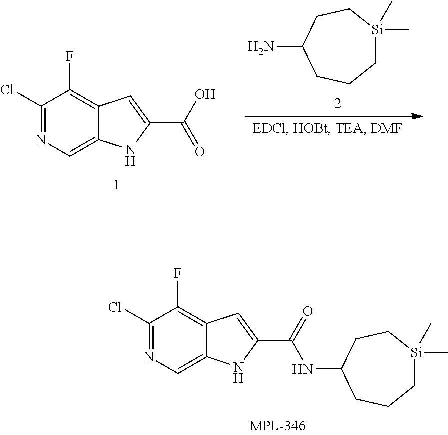

To a solution of 5-chloro-4-fluoro-1H-pyrrolo[2,3-c]pyridine-2-carboxylic acid (50 mg, 233.01 umol, 1 eq) and 1,1-dimethylsilepan-4-amine (54.19 mg, 279.61 umol, 1.2 eq, HCl salt) in DMF (1.5 mL) was added a solution of EDCI (134.01 mg, 699.04 umol, 3 eq) and HOBt (94.46 mg, 699.04 umol, 3 eq) in DMF (0.5 mL), followed by TEA (141.47 mg, 1.40 mmol, 194.59 uL, 6 eq) was added. The mixture was stirred at 20° C. for 1 hr. LC-MS indicated desired mass was detected. The reaction mixture was filtered to obtain filter residue, which was purified by prep-HPLC (column: YMC-Actus Triart C18 150*30 mm*5 um; mobile phase: A: 0.225% formic acid in water, B: CH₃CN, gradient: 65%-90% B over 11 min). Compound 5-chloro-N-(1,1-dimethylsilepan-4-yl)-4-fluoro-1H-pyrrolo[2,3-c]pyridine-2-carboxamide (28.8 mg, 81.38 umol, 34.93% yield, 100% purity) was obtained as a white solid.

LCMS (ESI) m/z: 354.1 [M+H]⁺; ¹H NMR (500 MHz, DMSO-d₆) δ=11.73-13.28 (m, 1H) 8.60-8.64 (m, 1H) 8.62 (br d, J=8.09 Hz, 1H) 8.45 (s, 1H) 7.39 (s, 1H) 3.88-3.95 (m, 1H) 1.79-1.96 (m, 3H) 1.65-1.74 (m, 1H) 1.45-1.56 (m, 2H) 0.70-0.82 (m, 2H) 0.58-0.66 (m, 2H) 0.04 (d, J=10.83 Hz, 6H).

### Example 180. MPL-348

### Synthesis of 5-chloro-4-fluoro-N-(6-silaspiro[5.5]undecan-3-yl)-1H-pyrrolo[2,3-c]pyridine-2-carboxamide

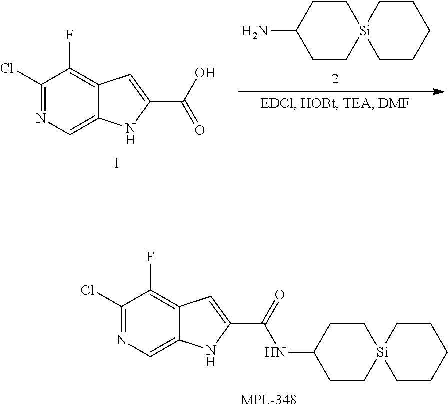 [SMILES: O=C(O)c1cc2c(F)c(Cl)ncc2[nH]1 ; NC1CC[Si]2(CCCCC2)CC1 ; O=C(NC1CC[Si]2(CCCCC2)CC1)c1cc2c(F)c(Cl)ncc2[nH]1]

To a solution of 5-chloro-4-fluoro-1H-pyrrolo[2,3-c]pyridine-2-carboxylic acid (50 mg, 233.01 umol, 1 eq) and 6-silaspiro[5.5]undecan-3-amine (61.47 mg, 279.61 umol, 1.2 eq, HCl salt) in DMF (1.5 mL) was added a solution of EDCI (134.01 mg, 699.04 umol, 3 eq) and HOBt (94.46 mg, 699.04 umol, 3 eq) in DMF (0.5 mL), followed by TEA (141.47 mg, 1.40 mmol, 194.59 uL, 6 eq). The mixture was stirred at 20° C. for 1 hr. LC-MS showed compound 1 was consumed completely. The mixture was filtered and the filtrate was purified by prep-HPLC (column: YMC-Actus Triart C18 150*30 mm*5 um; mobile phase: A: 0.225% formic acid in water, B: CH₃CN, gradient: 68%-97% B over 11 min.). Compound 5-chloro-4-fluoro-N-(6-silaspiro[5.5]undecan-3-yl)-1H-pyrrolo[2,3-c]pyridine-2-carboxamide (43.2 mg, 113.70 umol, 48.79% yield, 99.993% purity) was obtained as a white solid.

LCMS (ESI) m/z: 380.1 [M+H]⁺; ¹H NMR (500 MHz, DMSO-d6) δ=0.54-0.65 (m, 4H) 0.66-0.73 (m, 2H) 0.90 (br d, J=14.65 Hz, 2H) 1.38 (br s, 2H) 1.53-1.70 (m, 6H) 2.01 (br d, J=10.07 Hz, 2H) 3.70-3.79 (m, 1H) 7.35 (s, 1H) 8.44 (s, 1H) 8.59 (br d, J=7.93 Hz, 1H) 12.42-12.87 (m, 1H)

### Example 181. MPL-349

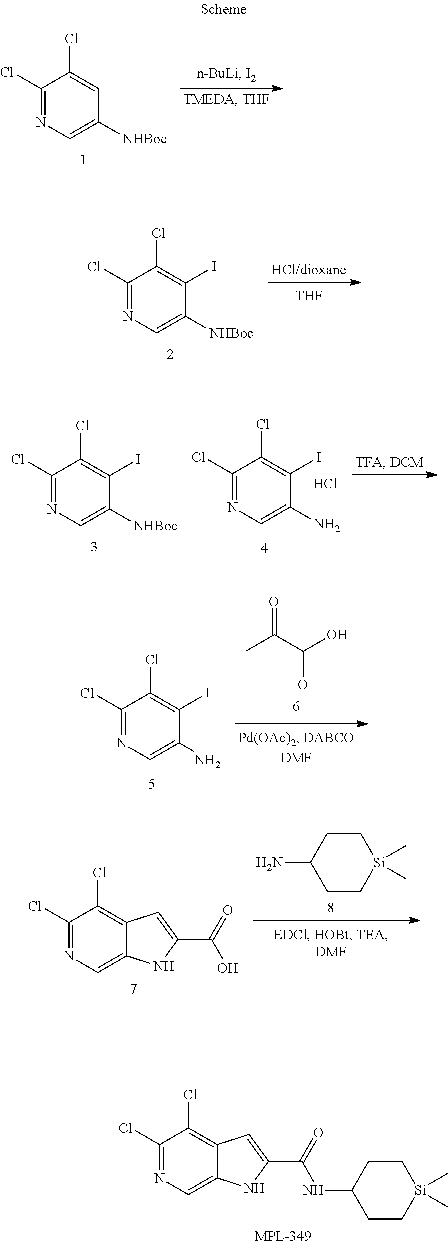

### Synthesis of tert-butyl N-(5,6-dichloro-4-iodo-3-pyridyl)carbamate

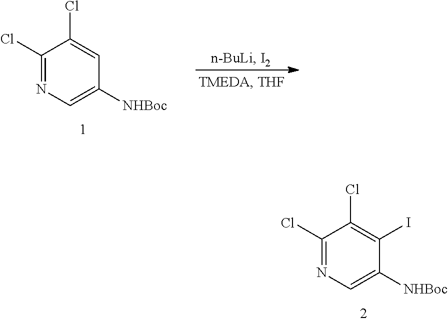

A mixture of tert-butyl N-(5,6-dichloro-3-pyridyl)carbamate (9 g, 34.20 mmol, 1 eq) in THE (100 mL) was degassed and purged with N₂ for 3 times, TMEDA (7.95 g, 68.41 mmol, 10.32 mL, 2 eq) and n-BuLi (2.5 M in hexane, 34.20 mL, 2.5 eq) was then added and the mixture was stirred at −60° C. for 30 min under N₂. A solution of I₂ (13.02 g, 51.31 mmol, 10.34 mL, 1.5 eq) in THE (20 mL) was added with stirring. The mixture was stirred at −60° C. for 30 min. TLC showed one major new spot with lower polarity. The reaction mixture was quenched with saturated Na₂SO₃ solution (100 mL) at 25° C., and then diluted with water (100 mL) and extracted with EtOAc (100 mL×2). The combined organic layer was washed with brine (100 mL×2), dried over Na₂SO₄, filtered and concentrated under reduced pressure. The resulting residue was purified by column chromatography (SiO₂, 0-5% Ethyl acetate in petroleum ether). Compound tert-butyl N-(5,6-dichloro-4-iodo-3-pyridyl)carbamate (12.46 g, 25.61 mmol, 68.07% yield, 80% purity) was obtained as a yellow solid. ¹H NMR was recorded.

### Synthesis of 5,6-dichloro-4-iodo-pyridin-3-amine

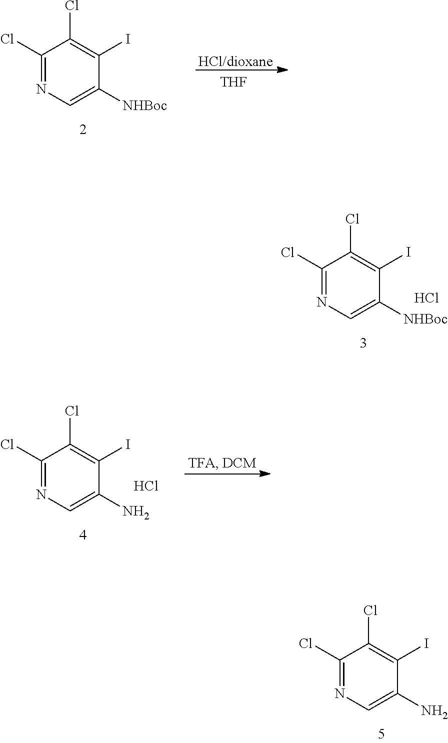

Step 1: To a solution of tert-butyl N-(5,6-dichloro-4-iodo-3-pyridyl)carbamate (12.46 g, 32.03 mmol, 1 eq) in THE (30 mL) was added HCl/dioxane (100 mL). The mixture was stirred at 25° C. for 1 hr. LCMS showed desired compound formed. The mixture was filtered. The cake was collected. ¹H NMR analysis indicated that the filter cake (10.6 g, yellow solid) was a mixture of compounds 3 and 4.

Step 2: A mixture of compound 3 and 4 (total 9.36 g) was diluted with water (50 mL) and neutralized with saturated NaHCO₃ to pH 8, and then extracted with EtOAc (30 mL×2). The combined organic layer was dried over Na₂SO₄ and concentrated under reduced pressure. The residue was dissolved in DCM (20 mL). TFA (8.24 g, 72.30 mmol, 5.35 mL, 8.62 eq) was added. The mixture was stirred at 25° C. for 1 hr. LCMS showed desired product. The mixture was adjusted to pH to 8 with saturated NaHCO₃, and then extracted with EtOAc (30 mL×2). The combined organic layer was dried over Na₂SO₄ and concentrated under reduced pressure. Compound 5,6-dichloro-4-iodo-pyridin-3-amine (3.8 g, crude) was obtained as a yellow solid. The crude product was used for the next step without further purification.

LCMS (ESI) m/z 288.8 [M+H]⁺; 1H NMR was recorded.

### Synthesis of 4,5-dichloro-1H-pyrrolo[2,3-c]pyridine-2-carboxylic acid

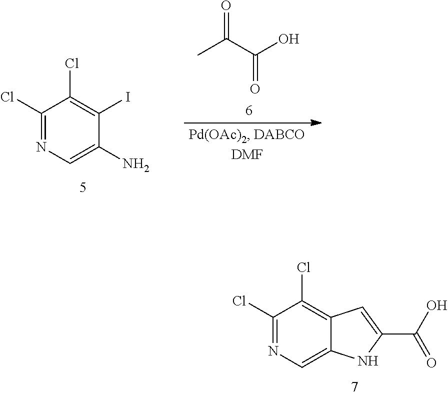

To a mixture of 5,6-dichloro-4-iodo-pyridin-3-amine (2 g, 6.92 mmol, 1 eq), 2-oxopropanoic acid (1.22 g, 13.85 mmol, 975.41 uL, 2 eq) and DABCO (1.55 g, 13.85 mmol, 1.52 mL, 2 eq) was added DMF (30 mL). The mixture was purged with N₂ and Pd(OAc)₂ (310.85 mg, 1.38 mmol, 0.2 eq) was added under N₂. The mixture was stirred at 110° C. for 4 hr. LCMS showed desired compound formed. The residue was filtered. The cake was washed with DMF (50 mL×3). The combined organic layer was triturated with toluene (50 mL) at 25° C. for 20 min and filtered. The cake was then triturated with water (50 mL) at 25° C. for 20 min and filtered. The cake was then triturated with CH₃CN (50 mL) at 25° C. for 20 min and filtered. The solid was collected by filtration. Compound 4,5-dichloro-1H-pyrrolo[2,3-c]pyridine-2-carboxylic acid (1.1 g, 4.52 mmol, 59.40% yield, 95% purity) was obtained as a brown solid.

LCMS (ESI) m/z 230.7 [M+H]⁺; ¹H NMR was recorded.

### Synthesis of 4,5-dichloro-N-(1,1-dimethylsilinan-4-yl)-1H-pyrrolo[2,3-c]pyridine-2-carboxamide

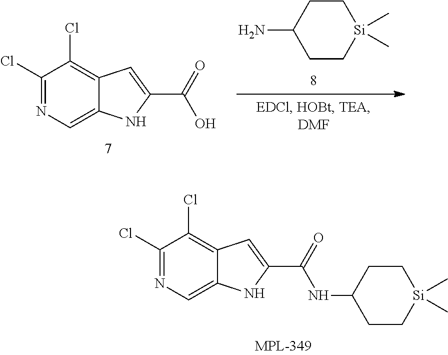

To a solution of 4,5-dichloro-1H-pyrrolo[2,3-c]pyridine-2-carboxylic acid (50 mg, 216.42 umol, 1 eq) and 1,1-dimethylsilinan-4-amine (46.68 mg, 259.70 umol, 1.2 eq, HCl) in DMF (1 mL), a solution of HOBt (87.73 mg, 649.25 umol, 3 eq) and EDCI (124.46 mg, 649.25 umol, 3 eq) in DMF (1 mL) was added, followed by TEA (109.50 mg, 1.08 mmol, 150.61 uL, 5 eq). The reaction mixture was stirred at 25° C. for 2 hr. LCMS showed the starting material was consumed completely. The mixture was purified by prep-HPLC (column: YMC-Actus Triart C18 150*30 mm*5 um; mobile phase: A: 0.225% formic acid in water, B: CH₃CN; gradient: 60%-90% B over 11 min). Compound 4,5-dichloro-N-(1,1-dimethylsilinan-4-yl)-1H-pyrrolo[2,3-c]pyridine-2-carboxamide (21.6 mg, 60.62 umol, 28.01% yield, 100% purity) was obtained as a yellow solid.

LCMS (ESI) m/z 356.0 [M+H]⁺; ¹H NMR (500 MHz, DMSO-d6) δ=12.61 (br s, 1H), 8.66 (br d, J=8.4 Hz, 1H), 8.54 (s, 1H), 7.35 (s, 1H), 3.74 (br d, J=8.4 Hz, 1H), 2.01 (br d, J=9.5 Hz, 2H), 1.68-1.53 (m, 2H), 0.79 (br d, J=14.8 Hz, 2H), 0.62 (dt, J=4.8, 14.1 Hz, 2H), 0.10 (s, 3H), 0.04 (s, 3H).

### Example 182. MPL-350

4,5-dichloro-N-(1,1-dimethylsilepan-4-yl)-1H-pyrrolo[2,3-c]pyridine-2-carboxamide

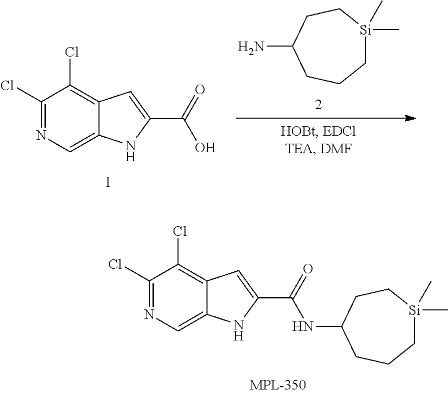

To a solution of 4,5-dichloro-1H-pyrrolo[2,3-c]pyridine-2-carboxylic acid (50 mg, 216.42 umol, 1 eq) and 1,1-dimethylsilepan-4-amine (50.33 mg, 259.70 umol, 1.2 eq, HCl) in DMF (1 mL), a solution of HOBt (87.73 mg, 649.25 umol, 3 eq) and EDCI (124.46 mg, 649.25 umol, 3 eq) in DMF (1 mL) was added with stirring, followed by TEA (109.50 mg, 1.08 mmol, 150.61 uL, 5 eq). The reaction mixture was stirred at 25° C. for 2 hr. LCMS showed desired compound formed. The mixture was purified by prep-HPLC (Gilson GX281, column: YMC-Actus Triart C18 150*30 mm*5 um; mobile phase: A: 0.225% formic acid in water, B: CH₃CN; gradient 65%-95% B over 11 min). Compound 4,5-dichloro-N-(1,1-dimethylsilepan-4-yl)-1H-pyrrolo[2,3-c]pyridine-2-carboxamide (19.7 mg, 53.19 umol, 24.58% yield, 100% purity) was obtained as a yellow solid.

LCMS (ESI) m/z 370.1 [M+H]⁺; ¹H NMR (500 MHz, DMSO-d6) δ=12.54 (br s, 1H), 8.64 (d, J=8.1 Hz, 1H), 8.49 (s, 1H), 7.32 (s, 1H), 4.04-3.74 (m, 1H), 1.91-1.73 (m, 3H), 1.71-1.58 (m, 1H), 1.52-1.37 (m, 2H), 0.78-0.63 (m, 2H), 0.62-0.52 (m, 2H), −0.01 (d, J=11.7 Hz, 6H).

### Example 183. MPL-318

### Synthesis of N-(1,1-dimethylsilinan-4-yl)-4-fluoro-5-methyl-1H-pyrrolo[2,3-c]pyridine-2-carboxamide

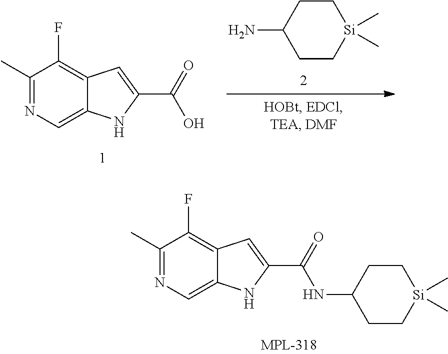

To a solution of 4-fluoro-5-methyl-1H-pyrrolo[2,3-c]pyridine-2-carboxylic acid (40 mg, 206.01 umol, 1 eq), 1,1-dimethylsilinan-4-amine (44.44 mg, 247.22 umol, 1.2 eq, HCl salt) in DMF (1 mL) was added HOBt (41.76 mg, 309.02 umol, 1.5 eq), EDCI (59.24 mg, 309.02 umol, 1.5 eq) and TEA (62.54 mg, 618.04 umol, 86.02 uL, 3 eq). The mixture was stirred at 25° C. for 1 hr. LC-MS showed desired mass. The reaction mixture was diluted with CH₃OH (2 mL) and filtered. The filtrate was purified by prep-HPLC (column: YMC-Actus Triart C18 150×30 mm×5 um; mobile phase: A: 0.225% formic acid in water, B: CH3CN; gradient: 35%-65% B over 11 min). Compound N-(1, 1-dimethylsilinan-4-yl)-4-fluoro-5-methyl-1H-pyrrolo[2,3-c]pyridine-2-carboxamide (24.2 mg, 74.87 umol, 36.34% yield, 98.8% purity) was obtained as a white solid.

LCMS (ESI) m/z 320.3 [M+H]⁺; ¹H NMR (400 MHz, METHANOL-d4) 6=8.52 (s, 1H), 7.19 (s, 1H), 3.79 (br t, J=11.5 Hz, 1H), 2.53 (d, J=3.1 Hz, 3H), 2.14 (br d, J=9.8 Hz, 2H), 1.75-1.62 (m, 2H), 0.89-0.81 (m, 2H), 0.77-0.65 (m, 2H), 0.13 (s, 3H), 0.05 (s, 3H).

### Example 184. MPL-366

### Synthesis of N-(1,1-dimethylsilepan-4-yl)-4-fluoro-5-(trifluoromethyl)-1H-pyrrolo[2,3-c]pyridine-2-carboxamide

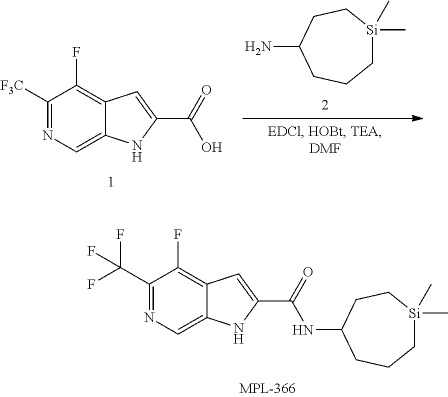

To a solution of 4-fluoro-5-(trifluoromethyl)-1H-pyrrolo[2,3-c]pyridine-2-carboxylic acid (60 mg, 241.80 umol, 1 eq) and 1,1-dimethylsilepan-4-amine (60.92 mg, 314.35 umol, 1.3 eq, HCl salt) in DMF (1 mL) was added a solution of EDCI (92.71 mg, 483.61 umol, 2 eq) and HOBt (65.35 mg, 483.61 umol, 2 eq) in DMF (1 mL), followed by TEA (97.87 mg, 967.22 umol, 134.62 uL, 4 eq). The mixture was stirred at 30° C. for 2 hr. LCMS showed desired mass. The mixture was filtered to remove insoluble matter. The filtrate was purified by prep-HPLC (column: YMC-Actus Triart C18 150*30 mm*5 um; mobile phase: A: 0.225% formic acid in water; B: CH₃CN; gradient: 65%-90% B over 11 min). Compound N-(1,1-dimethylsilepan-4-yl)-4-fluoro-5-(trifluoromethyl)-1H-pyrrolo[2,3-c]pyridine-2-carboxamide (8.8 mg, 22.71 umol, 9.39% yield, 100% purity) was obtained as a light yellow solid.

LCMS (ESI) m/z 388.1 [M+H]⁺; ¹H NMR (500 MHz, DMSO-d6) δ=13.33-12.20 (m, 1H), 8.65 (d, J=2.0 Hz, 1H), 8.62 (br d, J=8.1 Hz, 1H), 7.57-7.33 (m, 1H), 3.96-3.79 (m, 1H), 1.95-1.73 (m, 3H), 1.71-1.59 (m, 1H), 1.55-1.37 (m, 2H), 0.78-0.53 (m, 4H), −0.01 (d, J=11.0 Hz, 6H).

### Example 185. MPL-367

### Synthesis of 4-fluoro-N-(5-silaspiro[4.5]decan-8-yl)-5-(trifluoromethyl)-1H-pyrrolo[2,3-c]pyridine-2-carboxamide

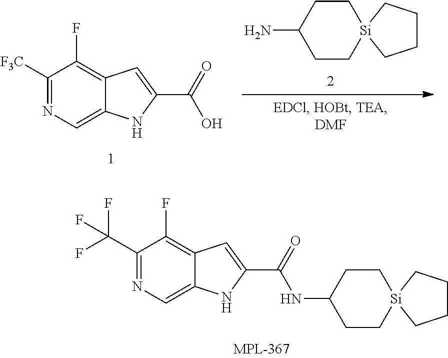 [SMILES: O=C(O)c1cc2c(F)c(C(F)(F)F)ncc2[nH]1 ; NC1CC[Si]2(CCCC2)CC1 ; O=C(NC1CC[Si]2(CCCC2)CC1)c1cc2c(F)c(C(F)(F)F)ncc2[nH]1]

To a solution of 4-fluoro-5-(trifluoromethyl)-1H-pyrrolo[2,3-c]pyridine-2-carboxylic acid (100 mg, 403.01 umol, 1 eq) and 5-silaspiro[4.5]decan-8-amine (99.53 mg, 483.61 umol, 1.2 eq, HCl salt) in DMF (1 mL) was added a solution of EDCI (154.51 mg, 806.02 umol, 2 eq) and HOBt (108.91 mg, 806.02 umol, 2 eq) in DMF (1 mL), followed by TEA (163.12 mg, 1.61 mmol, 224.38 uL, 4 eq). The mixture was stirred at 30° C. for 2 hr. LCMS showed desired mass. The reaction mixture was filtered to remove insoluble matter. The filtrate was purified by prep-HPLC (column: YMC-Actus Triart C18 150*30 mm*5 um; mobile phase: A: 0.225% formic acid in water, B: CH3CN; gradient: 67%-95% B over 11 min). Compound 4-fluoro-N-(5-silaspiro[4.5]decan-8-yl)-5-(trifluoromethyl)-1H-pyrrolo[2,3-c]pyridine-2-carboxamide (102 mg, 255.04 umol, 63.28% yield, 99.88% purity) was obtained as a brown solid.

LCMS (ESI) m/z 400.1 [M+H]⁺; ¹H NMR (500 MHz, DMSO-d6) δ=12.90 (br s, 1H), 8.71 (d, J=2.1 Hz, 1H), 8.67 (d, J=8.2 Hz, 1H), 7.54 (s, 1H), 3.86-3.73 (m, 1H), 2.17-2.03 (m, 2H), 1.69-1.50 (m, 6H), 0.86-0.69 (m, 4H), 0.66-0.44 (m, 4H).

### Example 186. MPL-368

### Synthesis of 4-fluoro-N-(6-silaspiro[5.5]undecan-3-yl)-5-(trifluoromethyl)-1H-pyrrolo[2,3-c]pyridine-2-carboxamide

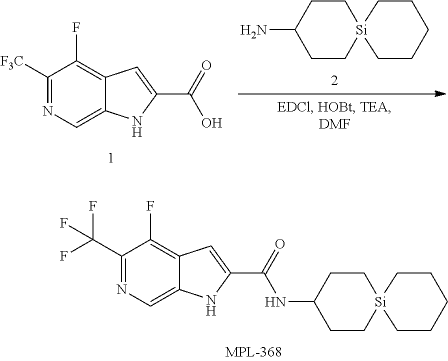 [SMILES: O=C(O)c1cc2c(F)c(C(F)(F)F)ncc2[nH]1 ; NC1CC[Si]2(CCCCC2)CC1 ; O=C(NC1CC[Si]2(CCCCC2)CC1)c1cc2c(F)c(C(F)(F)F)ncc2[nH]1]

To a solution of 4-fluoro-5-(trifluoromethyl)-1H-pyrrolo[2,3-c]pyridine-2-carboxylic acid (60 mg, 241.80 umol, 1 eq) and 6-silaspiro[5.5]undecan-3-amine (63.79 mg, 290.17 umol, 1.2 eq, HCl salt) in DMF (1 mL) was added a solution of EDCI (92.71 mg, 483.61 umol, 2 eq) and HOBt (65.35 mg, 483.61 umol, 2 eq) in DMF (1 mL), followed by TEA (97.87 mg, 967.22 umol, 134.62 uL, 4 eq). The mixture was stirred at 30° C. for 2 hr. LCMS showed desired mass. The mixture was filtered to remove insoluble matter. The filtrate was purified by prep-HPLC (column: YMC-Actus Triart C18 150*30 mm*5 um; mobile phase: A: 0.225% formic acid in water, B: CH₃CN; gradient: 70%-100% B over 11 min). Compound 4-fluoro-N-(6-silaspiro[5.5]undecan-3-yl)-5-(trifluoromethyl)-1H-pyrrolo[2,3-c]pyridine-2-carboxamide (16.8 mg, 40.63 umol, 16.80% yield, 100% purity) was obtained as a light yellow solid.

LCMS (ESI) m/z 414.1 [M+H]⁺; ¹H NMR (500 MHz, DMSO-d6) δ=12.91 (br s, 1H), 8.71 (d, J=2.0 Hz, 1H), 8.66 (br d, J=8.1 Hz, 1H), 7.65-7.34 (m, 1H), 3.76 (br d, J=8.2 Hz, 1H), 2.09-1.95 (m, 2H), 1.77-1.53 (m, 6H), 1.39 (br s, 2H), 0.92 (br d, J=14.6 Hz, 2H), 0.76-0.53 (m, 6H).

### Example 187. MPL-376

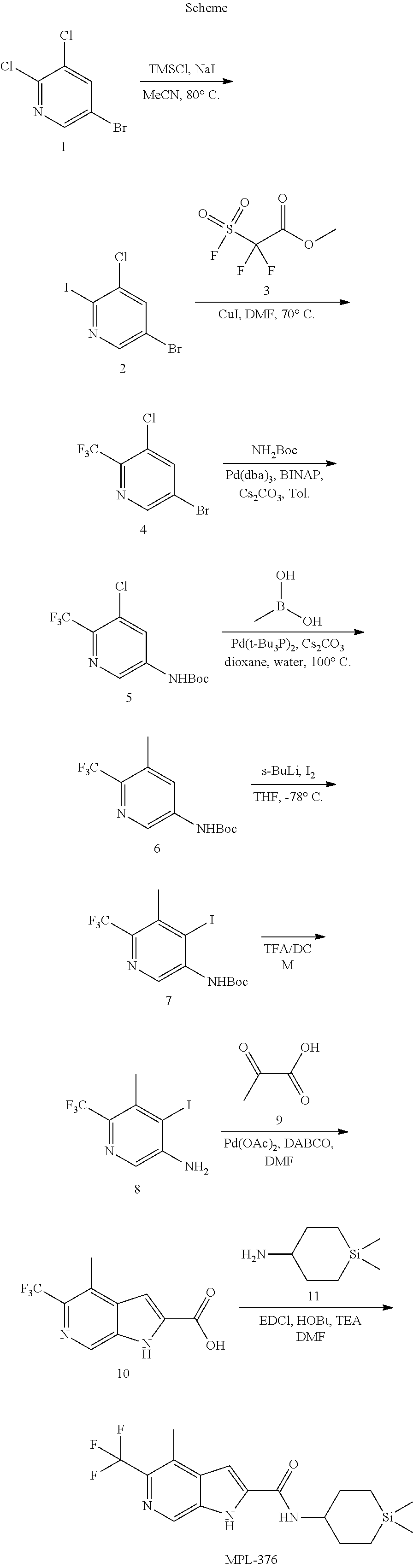

### Synthesis of 5-bromo-3-chloro-2-iodo-pyridine

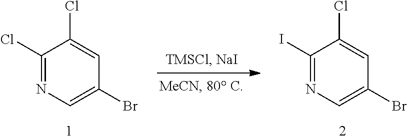 [SMILES: Clc1cc(Br)cnc1Cl ; Clc1cc(Br)cnc1I]

To a solution of 5-bromo-2,3-dichloro-pyridine (15 g, 66.11 mmol, 1 eq) and NaI (29.73 g, 198.34 mmol, 3 eq) in MeCN (100 mL) was added TMSCl (7.18 g, 66.11 mmol, 8.39 mL, 1 eq). The mixture was stirred at 80° C. under N₂ for 12 hr. TLC (petroleum ether:EtOAc=20:1) showed starting material was consumed completely, and one major new spot formed. The reaction mixture was poured into saturated Na₂SO₃ (500 mL). The mixture was concentrated under reduced pressure to remove MeCN, and then extracted with EtOAc (200 mL×2). The combined organic layer was washed with brine (100 mL×2), dried over Na₂SO₄, filtered and concentrated. The resulting residue was purified by flash silica gel chromatography (0-2% ethyl acetate in petroleum ether). Compound 5-bromo-3-chloro-2-iodo-pyridine (13 g, 24.50 mmol, 37.06% yield, 60% purity) was obtained as a white solid. ¹H NMR was recorded

### Synthesis of 5-bromo-3-chloro-2-(trifluoromethyl)pyridine

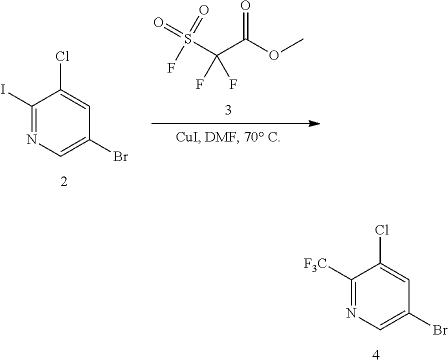

To a solution of 5-bromo-3-chloro-2-iodo-pyridine (10 g, 15.71 mmol, 1 eq) and methyl 2,2-difluoro-2-fluorosulfonyl-acetate (21.12 g, 109.95 mmol, 13.99 mL, 7 eq) in DMF (150 mL) was added CuI (20.94 g, 109.95 mmol, 7 eq). The mixture was stirred under N₂ at 60° C. for 12 hr. TLC (Petroleum ether:EtOAc=20:1) showed one major spot. The mixture was poured into water (1.5 L) and extracted with n-pentane (500 mL). The organic layer was washed with aqueous LiCl (3%, 100 mL×2) and brine (200 mL), dried over Na₂SO₄, filtered and concentrated under reduced pressure. The resulting residue was purified by flash silica gel chromatography (0-10% ethyl acetate in petroleum ether). Compound 5-bromo-3-chloro-2-(trifluoromethyl)pyridine (9 g, 13.82 mmol, 88.01% yield, 40% purity) was obtained as a colorless oil. ¹H NMR was recorded. The crude product was used for the next step without further purification.

### Synthesis of tert-butyl N-[5-chloro-6-(trifluoromethyl)-3-pyridyl]carbamate

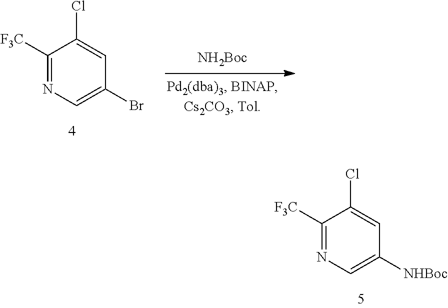 [SMILES: FC(F)(F)c1ncc(Br)cc1Cl ; CC(C)(C)OC(=O)Nc1cnc(C(F)(F)F)c(Cl)c1 ; "NH2Boc"]

To a mixture of 5-bromo-3-chloro-2-(trifluoromethyl)pyridine (7 g, 26.88 mmol, 1 eq), tert-butyl carbamate (9.45 g, 80.63 mmol, 3 eq) and Cs₂CO₃ (26.27 g, 80.63 mmol, 3 eq) in toluene (150 mL) was added Pd₂(dba)₃ (1.23 g, 1.34 mmol, 0.05 eq) and BINAP (1.67 g, 2.69 mmol, 0.1 eq) under N₂. The mixture was degassed with N₂ for 15 min and then stirred and refluxed at 110° C. for 12 hr. TLC (petroleum ether:EtOAc=8:1) showed one major spot. The mixture was filtered. The cake was washed by EtOAc (20 mL×3). The combined filtrate was concentrated under reduced pressure. The resulting residue was purified by flash silica gel chromatography (0-15% ethyl acetate in petroleum ether). Compound tert-butyl N-[5-chloro-6-(trifluoromethyl)-3-pyridyl]carbamate (3 g, 8.09 mmol, 30.10% yield, 80% purity) was obtained as a yellow solid. ¹H NMR was recorded.

### Synthesis of tert-butyl N-[5-methyl-6-(trifluoromethyl)-3-pyridyl]carbamate

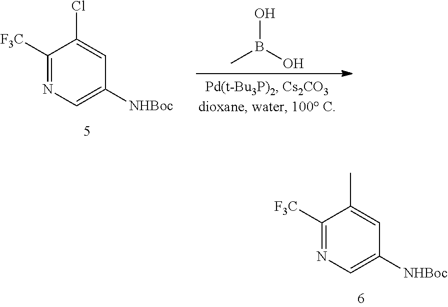

To a mixture of MeB(OH)₂ (6.05 g, 101.12 mmol, 10 eq), Cs₂CO₃ (6.44 g, 19.76 mmol, 1.95 eq) and Pd(t-Bu₃P)₂ (1.03 g, 2.02 mmol, 0.2 eq) was added a solution of tert-butyl N-[5-chloro-6-(trifluoromethyl)-3-pyridyl]carbamate (3 g, 10.11 mmol, 1 eq) in dioxane (100 mL) and H₂O (1 mL). The mixture was purged with N₂ and stirred at 100° C. for 12 hr under N₂. LCMS showed desired mass. The mixture was filtered. The cake was washed with EtOAc (20 mL×2). The combined filtrate was concentrated under reduced pressure. The resulting residue was purified by flash silica gel chromatography (0-20% ethyl acetate in petroleum ether). The fractions containing desired product (checked by TLC; petroleum ether:EtOAc=3:1) were collected and concentrated. The resulting residue was further purified by prep-HPLC (column: Xtimate C18 150*40 mm*5 um; mobile phase: A: 0.225% formic acid in water, B: CH₃CN, gradient: 52%-872% B over 8 min). Compound tert-butyl N-[5-methyl-6-(trifluoromethyl)-3-pyridyl]carbamate (1.3 g, 4.66 mmol, 46.07% yield, 99% purity) was obtained as a white solid. ¹H NMR was recorded.

### Synthesis of tert-butyl N-[4-iodo-5-methyl-6-(trifluoromethyl)-3-pyridyl]carbamate

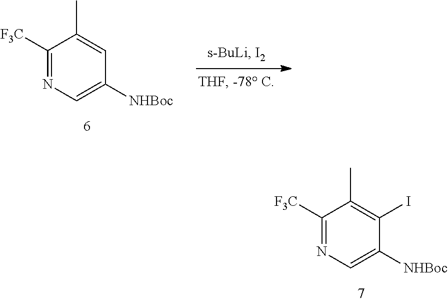 [SMILES: Cc1cc(NC(=O)OC(C)(C)C)cnc1C(F)(F)F ; Cc1c(C(F)(F)F)ncc(NC(=O)OC(C)(C)C)c1I]

To a solution of tert-butyl N-[5-methyl-6-(trifluoromethyl)-3-pyridyl]carbamate (1.1 g, 3.98 mmol, 1 eq) in THF (40) (dried by Na and distilled freshly) was added TMEDA (1.39 g, 11.95 mmol, 1.80 mL, 3 eq). The mixture was cooled to −78° C. under N₂. Then s-BuLi (0.9 M in n-hexane, 13.27 mL, 3 eq) was added dropwise, the mixture was stirred at −78° C. for 1 hr. A solution of I₂ (3.03 g, 11.95 mmol, 2.41 mL, 3 eq) in THE (10 mL) (dried with Na and distilled freshly) was added to the mixture dropwise, the mixture was stirred for 1 hr. TLC (Petroleum ether:EtOAc=5:1) showed starting material was remained, one spot with lower polarity formed. The reaction was quenched with Na₂SO₃ (Sat. 50 mL), the mixture was diluted with EtOAc (50 mL). The aqueous layer was extracted with EtOAc (30 mL). The combined organic layer was dried by Na₂SO₄, and then filtered and concentrated under reduced pressure. The resulting residue was purified by flash silica gel chromatography (0-7% Ethyl acetate in petroleum ether). Compound tert-butyl N-[4-iodo-5-methyl-6-(trifluoromethyl)-3-pyridyl]carbamate (450 mg, 1.06 mmol, 26.70% yield, 95% purity) was obtained as a white solid. ¹H NMR was recorded.

### Synthesis of 4-iodo-5-methyl-6-(trifluoromethyl)pyridin-3-amine

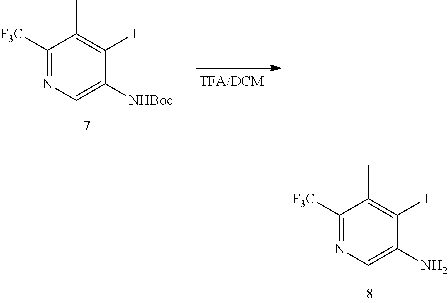 [SMILES: Cc1c(C(F)(F)F)ncc(NC(=O)OC(C)(C)C)c1I ; Cc1c(C(F)(F)F)ncc(N)c1I]

To a solution of tert-butyl N-[4-iodo-5-methyl-6-(trifluoromethyl)-3-pyridyl]carbamate (480 mg, 1.19 mmol, 1 eq) in DCM (5 mL) was added TFA (3.02 g, 26.46 mmol, 1.96 mL, 22.17 eq). The mixture was stirred at 20° C. for 3 hr. TLC (petroleum ether:EtOAc=3:1) showed starting material remained. Additional TFA (1 mL) was added. The mixture was stirred at 20° C. for 4 h. TLC (petroleum ether:EtOAc=3:1) showed starting material was consumed completely. The mixture was concentrated under reduced pressure. The resulting residue was diluted with saturated NaHCO₃ (20 mL), and then extracted with EtOAc (10 mL×2). The combined organic layer was dried by Na₂SO₄, and then filtered and concentrated under reduced pressure. Compound 4-iodo-5-methyl-6-(trifluoromethyl)pyridin-3-amine (380 mg, 1.13 mmol, 94.87% yield, 90% purity) was obtained as a brown solid. ¹H NMR was recorded.

### Synthesis of 4-methyl-5-(trifluoromethyl)-1H-pyrrolo[2,3-c]pyridine-2-carboxylic acid

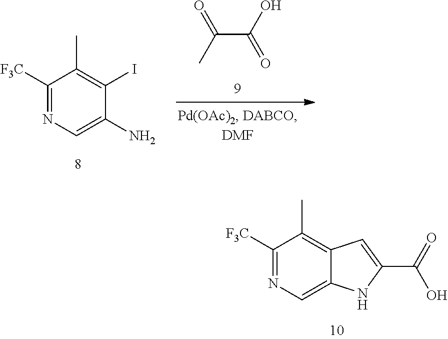

To a mixture of 4-iodo-5-methyl-6-(trifluoromethyl)pyridin-3-amine (350 mg, 1.16 mmol, 1 eq), 2-oxopropanoic acid (204.09 mg, 2.32 mmol, 163.27 uL, 2 eq) and DABCO (324.97 mg, 2.90 mmol, 318.60 uL, 2.5 eq) was added DMF (20 mL), followed by Pd(OAc)₂ (52.03 mg, 231.76 umol, 0.2 eq) under N₂. The mixture was stirred at 115° C. for 5 hr under N₂. LCMS showed one major peak with desired mass. The mixture was concentrated under reduced pressure. The resulting residue was diluted with MeOH (5 mL) and then filtered to remove insoluble matter. The filtrate was purified by prep-HPLC (column: YMC-Actus Triart C18 150*30 mm*5 um; mobile phase: A: 0.225% formic acid in water, B: CH₃CN; gradient: 25%-55% B over 11 min). Compound 4-methyl-5-(trifluoromethyl)-1H-pyrrolo[2,3-c]pyridine-2-carboxylic acid (190 mg, 739.24 umol, 63.79% yield, 95% purity) was obtained as a brown solid.

LCMS (ESI) m/z 245.0 [M+H]⁺; ¹H NMR (500 MHz, DMSO-d₆) δ=12.67 (br s, 1H), 8.71 (s, 1H), 7.42 (d, J=1.1 Hz, 1H), 2.66 (d, J=1.8 Hz, 3H).

### Synthesis of N-(1,1-dimethylsilinan-4-yl)-4-methyl-5-(trifluoromethyl)-1H-pyrrolo[2,3-c]pyridine-2-carboxamide

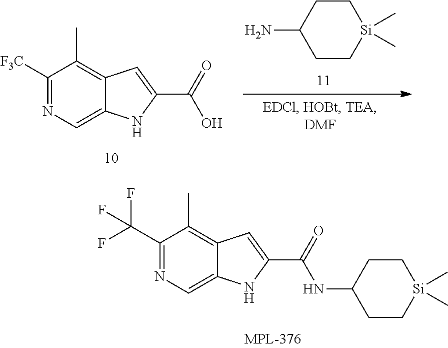

To a solution of 4-methyl-5-(trifluoromethyl)-1H-pyrrolo[2,3-c]pyridine-2-carboxylic acid (70 mg, 286.69 umol, 1 eq) and 1,1-dimethylsilinan-4-amine (61.84 mg, 344.02 umol, 1.2 eq, HCl salt) in DMF (2 mL) was added a solution of EDCI (109.92 mg, 573.37 umol, 2 eq) and HOBt (77.48 mg, 573.37 umol, 2 eq) in DMF (2 mL), followed by TEA (116.04 mg, 1.15 mmol, 159.61 uL, 4 eq). The mixture was stirred at 20° C. for 2 hr. LCMS showed one main peak with desired mass. The mixture was filtered to remove insoluble matter. The filtrate was purified by prep-HPLC (column: YMC-Actus Triart C18 150*30 mm*5 um; mobile phase: A: 0.225% formic acid in water, B: CH₃CN; gradient: 63%-93% B over 11 min). Compound N-(1,1-dimethylsilinan-4-yl)-4-methyl-5-(trifluoromethyl)-1H-pyrrolo[2,3-c]pyridine-2-carboxamide (63.5 mg, 171.87 umol, 59.95% yield, 100% purity) was obtained as a white solid.

LCMS (ESI) m/z 370.2 [M+H]⁺; ¹H NMR (500 MHz, DMSO-d₆) δ=12.35 (br s, 1H), 8.57 (s, 1H), 8.47 (d, J=8.1 Hz, 1H), 7.37 (s, 1H), 3.69-3.60 (m, 1H), 3.69-3.60 (m, 1H), 2.54 (d, J=1.7 Hz, 3H), 1.96-1.85 (m, 2H), 1.59-1.44 (m, 2H), 0.69 (br d, J=14.5 Hz, 2H), 0.53 (dt, J=4.7, 14.2 Hz, 2H), 0.03-0.11 (m, 6H).

### Example 188. MPL-379

### Synthesis of 5-bromo-2-iodo-3-methoxy-pyridine

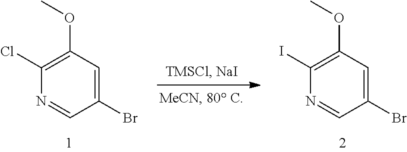

To a solution of 5-bromo-2-chloro-3-methoxy-pyridine (500 mg, 2.25 mmol, 1 eq) in MeCN (10 mL) was added NaI (1.01 g, 6.74 mmol, 3 eq), followed by TMSCl (244.17 mg, 2.25 mmol, 285.25 uL, 1 eq). The mixture was stirred at 80° C. for 12 hr. LCMS showed starting material was consumed completely, and desired mass was detected. The reaction mixture was poured into saturated Na₂SO₃ (10 mL). The mixture was concentrated under reduced pressure to remove MeCN, and then extracted by EtOAc (20 mL×2). The combined organic layer was washed with brine (20 mL×2), dried over Na₂SO₄, filtered and concentrated. The resulting residue was purified by flash silica gel chromatography (0-5% ethyl acetate in petroleum ether). The fractions containing desired product (checked by TLC; petroleum ether:EtOAc=20:1) were collected and concentrated. Compound 5-bromo-2-iodo-3-methoxy-pyridine (600 mg, 1.53 mmol, 68.03% yield, 80% purity) was obtained as a white solid. ¹H NMR was recorded.

### Step 2. Synthesis of 5-bromo-3-methoxy-2-(trifluoromethyl)pyridine

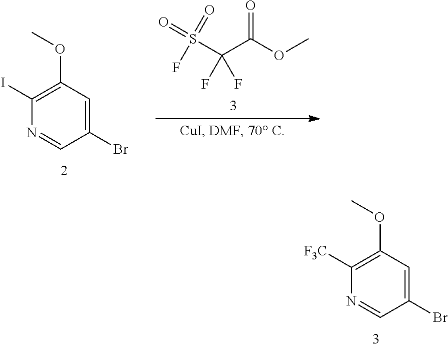

To a solution of 5-bromo-2-iodo-3-methoxy-pyridine (0.6 g, 1.91 mmol, 1 eq) in DMF (10 mL) was added methyl 2,2-difluoro-2-fluorosulfonyl-acetate (2.57 g, 13.38 mmol, 1.70 mL, 7 eq). CuI (2.55 g, 13.38 mmol, 7 eq) was then added under N₂. The mixture was stirred at 70° C. for 12 hr. LC-MS showed 80% of desired compound. The mixture was poured into water (100 mL), and then extracted with petroleum ether (30 mL). The organic layer was dried by Na₂SO₄, filtered and concentrated. The resulting residue was purified by flash silica gel chromatography (0-50% ethyl acetate in petroleum ether). The fractions containing desired product (check by TLC, petroleum ether:EtOAc=10:1, Rf=0.8) were combined and concentrated. Compound 5-bromo-3-methoxy-2-(trifluoromethyl)pyridine (350 mg, 1.23 mmol, 64.37% yield, 90% purity) was obtained as a colorless oil. ¹H NMR was recorded.

### Synthesis of tert-butyl N-[5-methoxy-6-(trifluoromethyl)-3-pyridyl]carbamate

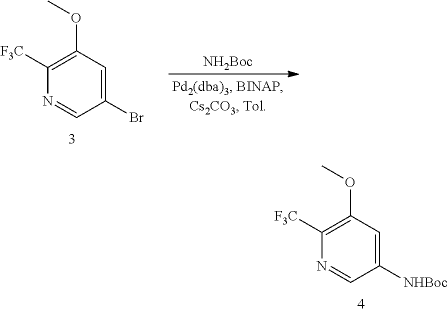 [SMILES: COc1cc(Br)cnc1C(F)(F)F ; COc1cc(NC(=O)OC(C)(C)C)cnc1C(F)(F)F ; "NH2Boc"]

To a mixture of 5-bromo-3-methoxy-2-(trifluoromethyl)pyridine (350 mg, 1.37 mmol, 1 eq), tert-butyl carbamate (480.44 mg, 4.10 mmol, 3 eq) and Cs₂CO₃ (1.34 g, 4.10 mmol, 3 eq) in toluene (10 mL) was added Pd₂(dba)₃ (125.19 mg, 136.71 umol, 0.1 eq) and BINAP (170.25 mg, 273.42 umol, 0.2 eq) under N₂. The mixture was degassed with N₂ for 15 min and then stirred and refluxed at 110° C. for 12 hr. LCMS showed starting material was consumed completely. The mixture was filtered. The cake was washed with EtOAc (20 mL×3). The combined filtrate was concentrated under reduced pressure. The resulting residues was purified by flash silica gel chromatography (0-25% ethyl acetate in petroleum ether). The fractions containing product (checked by TLC, petroleum ether:EtOAc=8:1) were collected and concentrated. Compound tert-butyl N-[5-methoxy-6-(trifluoromethyl)-3-pyridyl]carbamate (410 mg, 841.73 umol, 61.57% yield, 60% purity) was obtained as a yellow solid. ¹H NMR was recorded.

### Synthesis of tert-butyl N-[4-iodo-5-methoxy-6-(trifluoromethyl)-3-pyridyl]carbamate

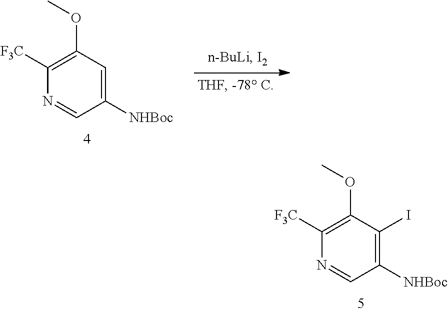 [SMILES: COc1cc(NC(=O)OC(C)(C)C)cnc1C(F)(F)F ; COc1c(C(F)(F)F)ncc(NC(=O)OC(C)(C)C)c1I]

To a solution of tert-butyl N-[5-methoxy-6-(trifluoromethyl)-3-pyridyl]carbamate (262 mg, 896.48 umol, 1 eq) in THF (10 mL) (dried with Na and distilled freshly) was added TMEDA (312.54 mg, 2.69 mmol, 405.90 uL, 3 eq). The mixture was cooled to −78° C. under N₂, n-BuLi (2.5 M in n-hexane, 1.97 mL, 5.5 eq) was then added dropwise. After stirring at −78° C. for 1 hr, a solution of I₂ (341.30 mg, 1.34 mmol, 270.87 uL, 1.5 eq) in THF (3 mL) (dried with Na and distilled freshly) was added dropwise, and the mixture was stirred for 1 hr. TLC (petroleum ether:EtOAc=5:1) showed one new spot with lower polarity. The mixture was warm to room temperature and quenched with saturated Na₂SO₃ (20 mL), the organic layer was separated. The aqueous layer was extracted with EtOAc (10 mL). The combined organic layer was dried with Na₂SO₄, and then filtered and concentrated under reduced pressure. The resulting residue was purified by flash silica gel chromatography (0-15% ethyl acetate in petroleum ether). Compound tert-butyl N-[4-iodo-5-methoxy-6-(trifluoromethyl)-3-pyridyl]carbamate (240 mg, 545.26 umol, 60.82% yield, 95% purity) was obtained as a white solid. ¹H NMR was recorded.

### Synthesis of ethyl 4-iodo-5-methoxy-6-(trifluoromethyl)pyridin-3-amine

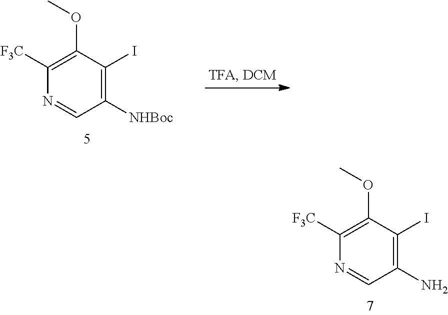 [SMILES: COc1c(C(F)(F)F)ncc(NC(=O)OC(C)(C)C)c1I ; COc1c(C(F)(F)F)ncc(N)c1I]

To a solution of tert-butyl N-[4-iodo-5-methoxy-6-(trifluoromethyl)-3-pyridyl]carbamate (240 mg, 573.96 umol, 1 eq) in DCM (1 mL) was added TFA (4.62 g, 40.52 mmol, 3 mL, 70.59 eq). The mixture was stirred at 20° C. for 2 hr. TLC (petroleum ether:EtOAc=5:1) indicated reactant was consumed completely and one new spot formed. The mixture was concentrated under reduced pressure. The residue was redissolved in EtOAc (10 mL) and washed with NaHCO₃ (10 mL×3). The organic layer was dried with Na₂SO₄, and then filtered and concentrated under reduced pressure. Compound 4-iodo-5-methoxy-6-(trifluoromethyl)pyridine-3-amine (200 mg, 565.98 umol, 98.61% yield, 90% purity) was obtained as a yellow solid. 1H NMR was recorded.

### Synthesis of 4-methoxy-5-(trifluoromethyl)-1H-pyrrolo[2,3-c]pyridine-2-carboxylic acid

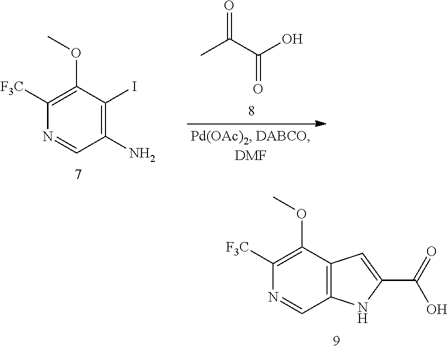

To a mixture of 4-iodo-5-methoxy-6-(trifluoromethyl)pyridin-3-amine (200 mg, 628.86 umol, 1 eq), 2-oxopropanoic acid (171.28 mg, 1.89 mmol, 15.16 uL, 97% purity, 3 eq) and DABCO (211.62 mg, 1.89 mmol, 207.47 uL, 3 eq) was added DMF (10 mL) (dried with CaH₂ and filtered). Pd(OAc)₂ (28.24 mg, 125.77 umol, 0.2 eq) was then added under N₂. The mixture was purged with N₂ for 15 min, and then stirred at 115° C. for 3 hr. LCMS showed one main peak with desired mass. The mixture was concentrated under reduced pressure. The residue was redissolved in MeOH and filtered to remove insoluble matter. The filtrate was purified by prep-HPLC (column: YMC-Actus Triart C18 150*30 mm*5 um; mobile phase: A: 0.225% formic acid in water, B: CH₃CN; gradient: 39%-69% B over 11 min). Compound 4-methoxy-5-(trifluoromethyl)-1H-pyrrolo[2,3-c]pyridine-2-carboxylic acid (50 mg, 182.57 umol, 29.03% yield, 95% purity) was obtained as a brown solid.

LCMS (ESI) m/z 261.0 [M+H]; ¹H NMR (500 MHz, DMSO-d₆) δ=12.82 (br s, 1H), 8.51 (s, 1H), 7.59-7.51 (m, 1H), 4.25 (s, 3H).

### Synthesis of N-(1,1-dimethylsilinan-4-yl)-4-methoxy-5-(trifluoromethyl)-1H-pyrrolo[2,3-c]pyridine-2-carboxamide

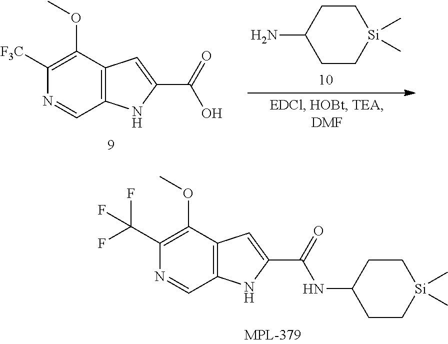

To a solution of 4-methoxy-5-(trifluoromethyl)-1H-pyrrolo[2,3-c]pyridine-2-carboxylic acid (30 mg, 115.31 umol, 1 eq) and 1,1-dimethylsilinan-4-amine (24.87 mg, 138.37 umol, 1.2 eq, HCl salt) in DMF (1 mL) was added a solution of EDCI (44.21 mg, 230.62 umol, 2 eq) and HOBt (31.16 mg, 230.62 umol, 2 eq) in DMF (1 mL), followed with TEA (46.67 mg, 461.24 umol, 64.20 uL, 4 eq). The mixture was stirred at 20° C. for 2 hr. LCMS showed one main peak with desired mass. The mixture was diluted with MeOH (2 mL) and filtered to remove insoluble matter. The filtrate was purified by prep-HPLC (column: YMC-Actus Triart C18 150*30 mm*5 um; mobile phase: A: 0.225% formic acid in water, B: CH₃CN; gradient 57%-87% B over 11 min). Compound N-(1,1-dimethylsilinan-4-yl)-4-methoxy-5-(trifluoromethyl)-1H-pyrrolo[2,3-c]pyridine-2-carboxamide (18.1 mg, 46.96 umol, 40.72% yield, 100% purity) was obtained as a white solid.

LCMS (ESI) m/z 386.1 [M+H]⁺; ¹H NMR (400 MHz, DMSO-d₆) δ=12.54 (br s, 1H), 8.50 (br d, J=8.3 Hz, 1H), 8.43 (s, 1H), 7.67 (s, 1H), 4.20 (s, 3H), 3.79-3.62 (m, 1H), 1.99 (br d, J=9.5 Hz, 2H), 1.62-1.49 (m, 2H), 0.80-0.70 (m, 2H), 0.66-0.53 (m, 2H), 0.06 (s, 3H), 0.00 (s, 3H).

### Example 189. MPL-382

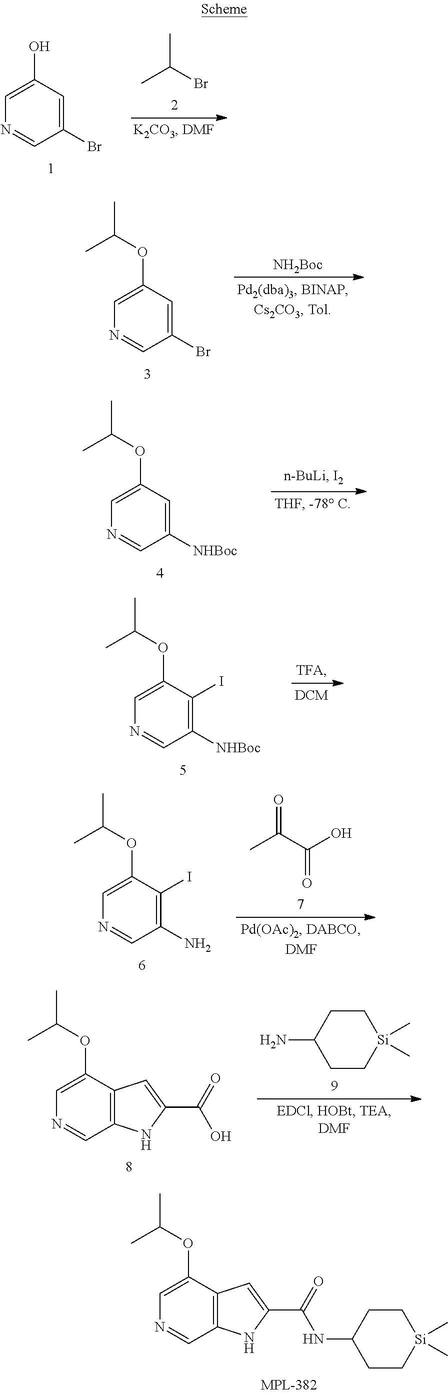

### Synthesis of 3-bromo-5-isopropoxy-pyridine

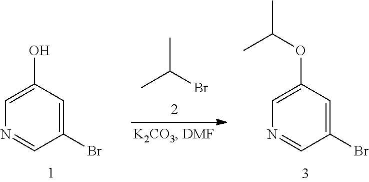

To a solution of 5-bromopyridin-3-ol (2 g, 11.49 mmol, 1 eq) in DMF (10 mL) was added K₂CO₃ (3.18 g, 22.99 mmol, 2 eq) and 2-bromopropane (2.83 g, 22.99 mmol, 2.16 mL, 2 eq). The mixture was stirred at 100° C. for 12 hr. TLC showed that the starting material was consumed completely, and one new spot formed. The mixture was poured into a mixture of H₂O (100 mL) and EtOAc (100 mL). Aqueous layer was extracted with EtOAc (2×50 mL). The combined organic layer was dried over Na₂SO₄ and concentrated under reduce pressure. The residue was purified by column chromatography (SiO₂, 0-20% ethyl acetate in petroleum ether). Compound 3-bromo-5-isopropoxy-pyridine (2.2 g, 9.16 mmol, 79.72% yield, 90% purity) was obtained as a white solid. ¹H NMR was recorded.

### Synthesis of tert-butyl N-(5-isopropoxy-3-pyridyl)carbamate

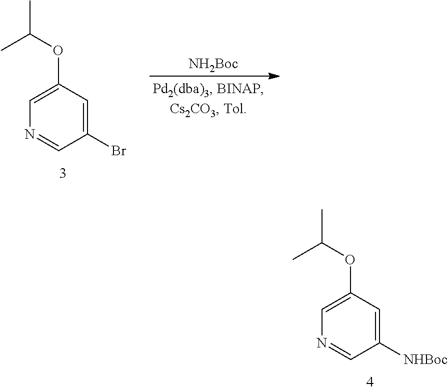 [SMILES: CC(C)Oc1cncc(Br)c1 ; CC(C)Oc1cncc(NC(=O)OC(C)(C)C)c1 ; "NH2Boc"]

To a mixture of 3-bromo-5-isopropoxy-pyridine (2.2 g, 10.18 mmol, 1 eq) and tert-butyl carbamate (2.39 g, 20.36 mmol, 2 eq) in toluene (30 mL) was added Cs₂CO₃ (6.63 g, 20.36 mmol, 2 eq) and Pd(dba)₂ (585.45 mg, 1.02 mmol, 0.1 eq) and BINAP (1.27 g, 2.04 mmol, 0.2 eq) under N₂. The mixture was stirred at 110° C. for 16 hr under N₂. LCMS showed desired mass. The reaction mixture was concentrated under reduced pressure. The residue was diluted with water (50 mL), and then extracted with EtAOC (50 mL×2). The combined organic layer was dried over Na₂SO₄ and concentrated under reduced pressure. The resulting residue was purified by column chromatography (SiO₂, 0-33% Ethyl acetate in petroleum ether). Compound tert-butyl N-(5-isopropoxy-3-pyridyl) carbamate (1.1 g, 4.14 mmol, 40.68% yield, 95% purity) was obtained as a white solid.

LCMS (ESI) m/z: 253.2 [M+H]⁺; ¹H NMR was recorded.

### Step 3. Synthesis of tert-butyl N-(4-iodo-5-isopropoxy-3-pyridyl)carbamate

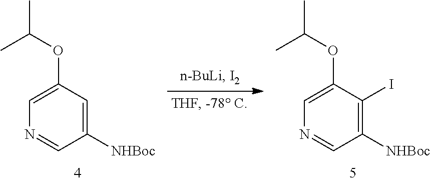 [SMILES: CC(C)Oc1cncc(NC(=O)OC(C)(C)C)c1 ; CC(C)Oc1cncc(NC(=O)OC(C)(C)C)c1I]

To a solution of tert-butyl N-(5-isopropoxy-3-pyridyl) carbamate (1.1 g, 4.36 mmol, 1 eq) and TMEDA (1.01 g, 8.72 mmol, 1.32 mL, 2 eq) in THF (10 mL) was added n-BuLi (2.5 M in n-hexane, 5.23 mL, 3 eq) dropwise at −78° C. under N₂. After stirring at −78° C. for 30 min, A solution of I₂ (1.66 g, 6.54 mmol, 1.32 mL, 1.5 eq) in THF (10 mL) was added dropwise at −78° C. The reaction mixture was stirred at −78° C. for another 30 min. TLC indicated a new spot was formed and some starting material remained. The reaction mixture was quenched with saturated Na₂SO₃ (30 mL) at 25° C., and then diluted with H₂O (30 mL) and extracted with EtOAc (50 mL×2). The combined organic layer was washed with brine (50 mL), dried over Na₂SO₄, and then filtered and concentrated under reduced pressure. The residue was purified by column chromatography (SiO₂, 0-20% ethyl acetate in petroleum ether). Compound tert-butyl N-(4-iodo-5-isopropoxy-3-pyridyl)carbamate (1 g, 2.12 mmol, 48.52% yield, 80% purity) was obtained as a white solid. ¹H NMR was recorded.

### Synthesis of 4-iodo-5-isopropoxy-pyridin-3-amine

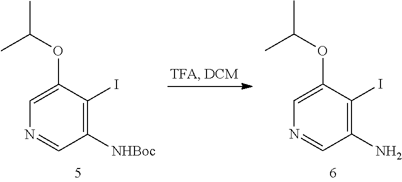 [SMILES: CC(C)Oc1cncc(NC(=O)OC(C)(C)C)c1I ; CC(C)Oc1cncc(N)c1I]

To a solution of tert-butyl N-(4-iodo-5-isopropoxy-3-pyridyl) carbamate (1.03 g, 2.73 mmol, 1 eq) in DCM (10 mL) was added TFA (15.40 g, 135.06 mmol, 10 mL, 49.47 eq). The mixture was stirred at 20° C. for 1 hr. TLC showed that reactant 5 was consumed and a new spot formed. The reaction mixture was concentrated under reduce pressure. Saturated NaHCO₃ (10 mL) was added to the residue and the mixture was extracted with DCM (20 mL×2). The combined organic layer was dried with Na₂SO₄ and concentrated under reduce pressure. Compound 4-iodo-5-isopropoxy-pyridin-3-amine (650 mg, 2.10 mmol, 77.06% yield, 90% purity) was obtained as a yellow solid. ¹H NMR was recorded.

### Synthesis of 4-isopropoxy-1H-pyrrolo[2,3-c]pyridine-2-carboxylic acid

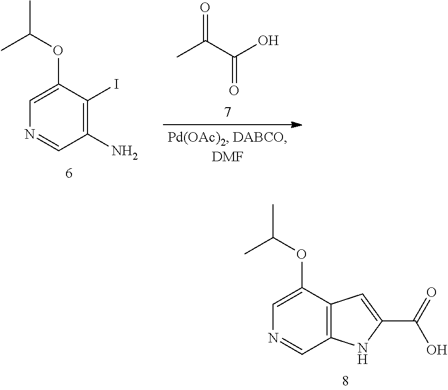

A mixture of 4-iodo-5-isopropoxy-pyridin-3-amine (300 mg, 1.08 mmol, 1 eq), 2-oxopropanoic acid (190.00 mg, 2.16 mmol, 152.00 uL, 2 eq), and DABCO (242.02 mg, 2.16 mmol, 237.28 uL, 2 eq) in DMF (5 mL) was degassed and purged with N₂ for 3 times. Pd(OAc)₂ (50 mg, 222.71 umol, 2.06e-1 eq) was then added. The mixture was stirred at 110° C. for 4 hr under N₂ atmosphere. LCMS showed desired mass. The reaction mixture was filtered. The filtrate was concentrated under reduced pressure to remove DMF. The residue was diluted with toluene (30 mL). The suspension was sonicated for 30 min. The supernatant was then poured off. The residue was diluted with H₂O (10 mL), adjusted to pH to 3-4 using aqueous HCl (1 N), and filtered. The solid was collected and dried. Compound 4-isopropoxy-1H-pyrrolo[2,3-c]pyridine-2-carboxylic acid (100 mg, 431.38 umol, 39.99% yield, 95% purity) was obtained as a yellow solid.

LCMS m/z: 221.1 [M+1]⁺; ¹H NMR (400 MHz, METHANOL-d₄) δ=8.70 (s, 1H), 7.95 (s, 1H), 7.40 (s, 1H), 4.97-4.90 (m, 1H), 1.48 (s, 3H), 1.47 (br s, 3H).

### Synthesis of N-(1,1-dimethylsilinan-4-yl)-4-Isopropoxy-1H-pyrrolo[2,3-c]pyridine-2-carboxamide

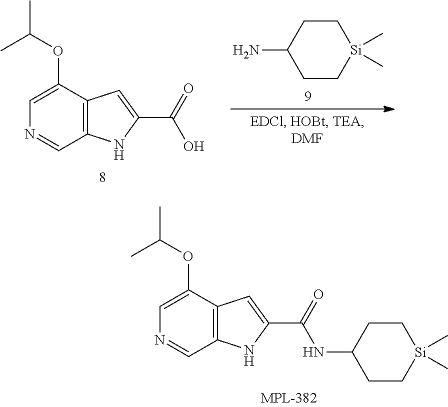

To a solution of 4-isopropoxy-1H-pyrrolo[2,3-c]pyridine-2-carboxylic acid (30 mg, 136.22 umol, 1 eq) and 1,1-dimethylsilinan-4-amine (24.49 mg, 136.22 umol, 1 eq, HCl salt) in DMF (0.5 mL) was added a solution of HOBt (55.22 mg, 408.67 umol, 3 eq) and EDCI (78.34 mg, 408.67 umol, 3 eq) in DMF (0.5 mL), followed by TEA (82.71 mg, 817.35 umol, 113.76 uL, 6 eq). The mixture was stirred at 20° C. for 1 hr. LCMS showed desired mass. The reaction mixture was filtered. The filtrate was purified by prep-HPLC (YMC-Actus Triart C18 150*30 mm*5 um; mobile phase: A: 0.225% formic acid in water, B: CH₃CN; gradient: 35%-65% B over 11 min). Compound N-(1,1-dimethylsilinan-4-yl)-4-isopropoxy-1H-pyrrolo[2,3-c]pyridine-2-carboxamide (9 mg, 26.05 umol, 19.12% yield, 100% purity) was obtained as a white solid.

LCMS (ESI) m/z: 346.2 [M+H]⁺; ¹H NMR (500 MHz, METHANOL-d4) 6=8.46 (s, 1H), 7.78 (s, 1H), 7.30 (s, 1H), 3.83-3.75 (m, 1H), 2.17-2.10 (m, 2H), 1.73-1.63 (m, 2H), 1.44 (s, 3H), 1.43 (s, 3H), 0.88-0.82 (m, 2H), 0.75-0.68 (m, 2H), 0.13 (s, 3H), 0.05 (s, 3H).

### Example 190: MPL-402

### Synthesis of N-(1,1-dimethylsilolan-3-yl)-5-(trifluoromethyl)-1H-pyrrolo[2,3-c]pyridine-2-carboxamide

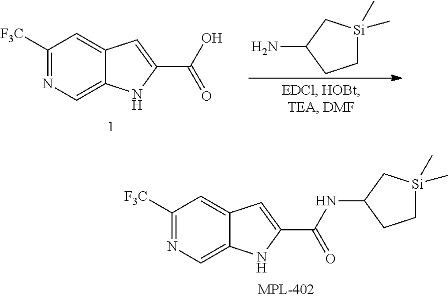

To a solution of 5-(trifluoromethyl)-1H-pyrrolo[2,3-c]pyridine-2-carboxylic acid (30 mg, 130.35 umol, 1 eq) and 1,1-dimethylsilolan-3-amine (23.76 mg, 143.39 umol, 1.1 eq, HCl salt) in DMF (1 mL) was added a solution of EDCI (49.98 mg, 260.71 umol, 2 eq) and HOBt (35.23 mg, 260.71 umol, 2 eq), followed by TEA (52.76 mg, 521.41 umol, 72.57 uL, 4 eq). The mixture was stirred at 20° C. for 2 hr. LC-MS showed one main peak with desired mass. The mixture was diluted with MeOH (2 mL) and filtered to remove insoluble matter. The filtrate was purified by prep-HPLC (column: YMC-Actus Triart C18 150*30 mm*5 um; mobile phase: A: 0.225% formic acid in water, B: CH₃CN; gradient: 56%-86% B over 11 min. Compound N-(1,1-dimethylsilolan-3-yl)-5-(trifluoromethyl)-1H-pyrrolo[2,3-c]pyridine-2-carboxamide (21.2 mg, 62.10 umol, 47.64% yield, 100% purity) was obtained as a white solid.

LCMS (ESI) m/z 342.0 [M+H]⁺; ¹H NMR (400 MHz, DMSO-d₆) δ=12.34 (s, 1H), 8.69 (s, 1H), 8.50 (br d, J=7.8 Hz, 1H), 8.03 (s, 1H), 7.19 (s, 1H), 3.95-3.81 (m, 1H), 1.95-1.80 (m, 1H), 1.28 (dq, J=7.1, 12.1 Hz, 1H), 1.01-0.90 (m, 1H), 0.72-0.58 (m, 1H), 0.50 (dd, J=11.4, 14.1 Hz, 1H), 0.42-0.26 (m, 1H), 0.00 (s, 6H).

### Example 191. MPL-445

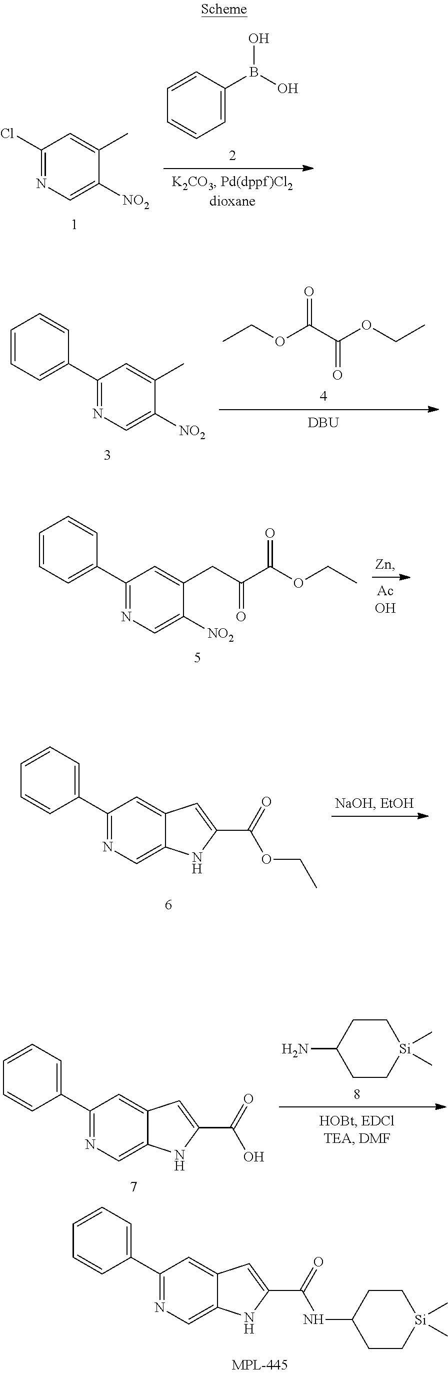

### Synthesis of 4-methyl-5-nitro-2-phenyl-pyridine

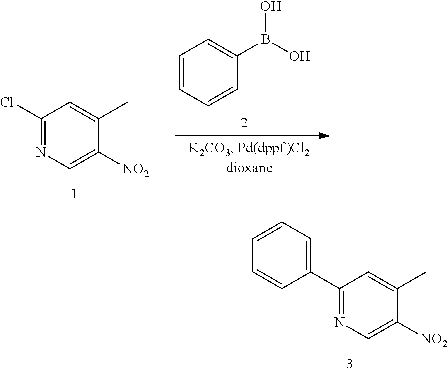 [SMILES: Cc1cc(Cl)ncc1[N+](=O)[O-] ; OB(O)c1ccccc1 ; Cc1cc(-c2ccccc2)ncc1[N+](=O)[O-]]

To a mixture of 2-chloro-4-methyl-5-nitro-pyridine (5 g, 28.97 mmol, 1 eq), phenylboronic acid (4.3 g, 35.27 mmol, 1.22 eq) and K₂CO₃ (8.01 g, 57.95 mmol, 2 eq) was added dioxane (50 mL) and H₂O (1 mL). The mixture was purged with N₂ and then Pd(dppf)Cl₂.CH₂Cl₂ (2.37 g, 2.90 mmol, 0.1 eq) was added under N₂. The mixture was stirred at 110° C. for 12 hr. LCMS showed desired mass. The mixture was filtered. The cake was washed with EtOAc (50 mL×2). The combined filtrate was dried over Na₂SO₄, and then concentrated in vacuo. The residue was purified by column chromatography (SiO₂, 0-3% ethyl acetate in petroleum ether). Compound 4-methyl-5-nitro-2-phenyl-pyridine (4 g, 15.87 mmol, 54.78% yield, 85% purity) was obtained as a red solid.

LCMS (ESI) m/z 215.1 [M+H]⁺; ¹H NMR was recorded.

### Synthesis of ethyl 3-(5-nitro-2-phenyl-4-pyridyl)-2-oxo-propanoate

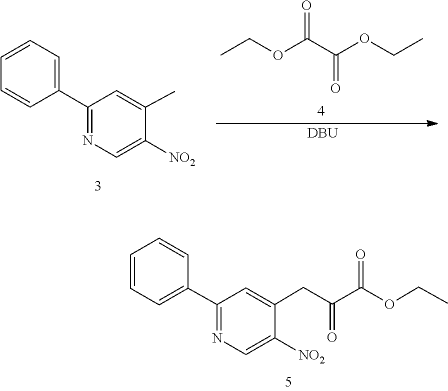

To a solution of 4-methyl-5-nitro-2-phenyl-pyridine (1 g, 4.67 mmol, 1 eq) in diethyl oxalate (10.70 g, 73.22 mmol, 10 mL, 15.68 eq) was added DBU (2.84 g, 18.67 mmol, 2.81 mL, 4 eq). The mixture was stirred at 40° C. for 12 hr. LCMS showed desired mass. The residue was diluted with water (50 mL) and extracted with EtOAc (50 mL×3). The combined organic layer was washed with brine (50 mL×2), dried over Na₂SO₄, and then filtered and concentrated under reduced pressure. The residue was purified by column chromatography (SiO₂, 0-10% ethyl acetate in petroleum ether). Compound ethyl 3-(5-nitro-2-phenyl-4-pyridyl)-2-oxo-propanoate (683 mg, 1.74 mmol, 37.24% yield, 80% purity) was obtained as a yellow solid.

LCMS (ESI) m/z 315.1 [M+H]⁺; ¹H NMR was recorded.

### Synthesis of ethyl 5-phenyl-1H-pyrrolo[2,3-c]pyridine-2-carboxylate

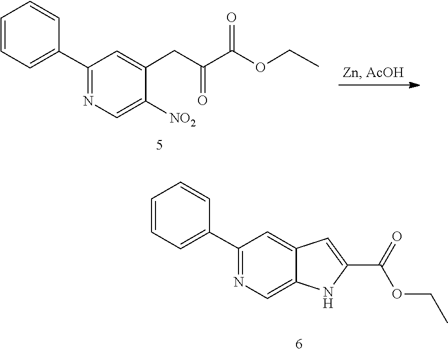 [SMILES: CCOC(=O)C(=O)Cc1cc(-c2ccccc2)ncc1[N+](=O)[O-] ; CCOC(=O)c1cc2cc(-c3ccccc3)ncc2[nH]1]

To a solution of ethyl 3-(5-nitro-2-phenyl-4-pyridyl)-2-oxo-propanoate (800 mg, 2.55 mmol, 1 eq) in THE (10 mL) was added Zn (1.66 g, 25.45 mmol, 10 eq) and AcOH (764.28 mg, 12.73 mmol, 727.89 uL, 5 eq). The mixture was stirred at 75° C. for 2 hr. LCMS showed desired mass. The reaction mixture was filtered and concentrated under reduced pressure. The residue was purified by column chromatography (SiO₂, 0-5% methanol in dichloromethane). Compound Ethyl 5-phenyl-1H-pyrrolo[2,3-c]pyridine-2-carboxylate (328 mg, 985.38 umol, 38.71% yield, 80% purity) was obtained as a yellow solid.

LCMS (ESI) m/z 267.1 [M+H]⁺; ¹H NMR was recorded.

### Synthesis of 5-phenyl-1H-pyrrolo[2,3-c]pyridine-2-carboxylic acid

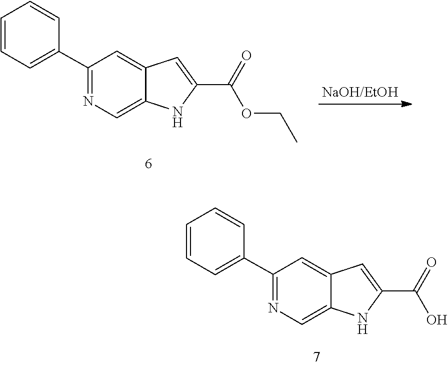 [SMILES: CCOC(=O)c1cc2cc(-c3ccccc3)ncc2[nH]1 ; O=C(O)c1cc2cc(-c3ccccc3)ncc2[nH]1]

To a solution of ethyl 5-phenyl-1H-pyrrolo[2,3-c]pyridine-2-carboxylate (50 mg, 187.76 umol, 1 eq) in EtOH (2 mL) was added NaOH (2 M, 2 mL, 21.30 eq). The mixture was stirred at 80° C. for 12 hr. LCMS showed desired mass. The reaction mixture was concentrated under reduced pressure to remove EtOH. The residue was diluted with water (20 mL), adjusted to pH to 2 using aqueous HCl (2 M), and then extracted with EtOAc (20 mL×2). The combined organic layer was washed with brine (20 mL×2), dried over Na₂SO₄, filtered and concentrated under reduced pressure. Compound 5-phenyl-1H-pyrrolo[2,3-c]pyridine-2-carboxylic acid (40 mg, 134.32 umol, 71.54% yield, 80% purity) was obtained as a yellow solid, which was used for the next step without further purification.

LCMS (ESI) m/z 239.1 [M+H]⁺; ¹H NMR (400 MHz, DMSO-d6) δ=13.10 (br s, 1H), 8.99 (s, 1H), 8.43 (s, 1H), 8.11-8.02 (m, 2H), 7.60-7.45 (m, 3H), 7.32 (s, 1H).

### N-(1,1-dimethylsilinan-4-yl)-5-phenyl-1H-pyrrolo[2,3-c]pyridine-2-carboxamide

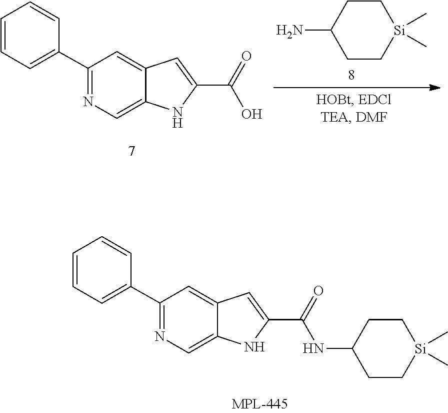

To a solution of 5-phenyl-1H-pyrrolo[2,3-c]pyridine-2-carboxylic acid (40 mg, 167.90 umol, 1 eq) and 1,1-dimethylsilinan-4-amine (33.20 mg, 184.69 umol, 1.1 eq, HCl salt) in DMF (1 mL) was added a solution of HOBt (68.06 mg, 503.69 umol, 3 eq) and EDCI (96.56 mg, 503.69 umol, 3 eq) in DMF (1 mL) with stirring, followed by TEA (84.95 mg, 839.49 umol, 116.85 uL, 5 eq) The mixture was stirred at 25° C. for 2 hr. LCMS showed desired mass. The mixture was purified by prep-HPLC (column: Phenomenex Synergi C18 150*30 mm*4 um; mobile phase: A: 0.225% formic acid in water, B: CH₃CN; gradient: 28%-59% B over 11 min). Compound N-(1,1-dimethylsilinan-4-yl)-5-phenyl-1H-pyrrolo[2,3-c]pyridine-2-carboxamide (34.3 mg, 87.75 umol, 52.26% yield, 93% purity) was obtained as a white solid.

LCMS (ESI) m/z 364.2 [M+H]⁺; ¹H NMR (500 MHz, DMSO-d6) δ=12.29 (br s, 1H), 8.86 (s, 1H), 8.58 (br s, 1H), 8.26 (br d, J=8.2 Hz, 1H), 8.12-7.96 (m, 2H), 7.58-7.22 (m, 4H), 3.75 (br d, J=8.5 Hz, 1H), 2.10-1.95 (m, 2H), 1.73-1.53 (m, 2H), 0.87-0.56 (m, 4H), 0.17-−0.03 (m, 6H).

### Example 192. MPL-451

### Synthesis of N-(1, 1-dimethylsilepan-4-yl)-4-methyl-5-(trifluoromethyl)-1H-pyrrolo[2,3-c]pyridine-2-carboxamide

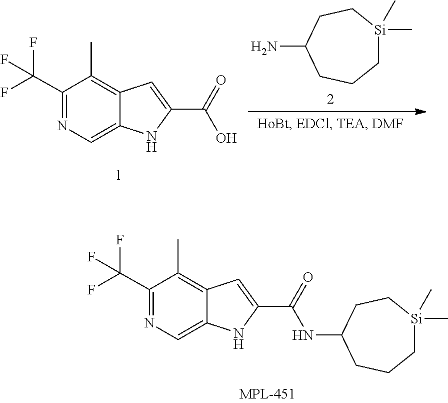

To a solution of 4-methyl-5-(trifluoromethyl)-1H-pyrrolo[2,3-c]pyridine-2-carboxylic acid (50 mg, 204.78 umol, 1 eq) and 1,1-dimethylsilepan-4-amine (47.62 mg, 245.73 umol, 1.2 eq, HCl salt) in DMF (1.5 mL) was added a solution of EDCI (117.77 mg, 614.33 umol, 3 eq) and HOBt (83.01 mg, 614.33 umol, 3 eq) in DMF (1.5 mL), followed by TEA (103.61 mg, 1.02 mmol, 142.51 uL, 5 eq). The mixture was stirred at 25° C. for 1 hr. LCMS showed desired mass. The reaction mixture was filtered. The filtrate was purified by prep-HPLC (column: Phenomenex Synergi C18 150*30 mm*4 um; mobile phase: A: 0.225% formic acid in water, B: CH₃CN, gradient: 53%-83% B over 11 min). Compound N-(1, 1-dimethylsilepan-4-yl)-4-methyl-5-(trifluoromethyl)-1H-pyrrolo[2,3-c]pyridine-2-carboxamide (24.9 mg, 64.66 umol, 31.57% yield, 99.6% purity) was obtained as a white solid.

LCMS m/z: 384.2 [M+1]⁺; ¹H NMR (400 MHz, DMSO-d₆) δ=12.43 (br s, 1H), 8.67 (s, 1H), 8.59 (br d, J=8.1 Hz, 1H), 7.49 (s, 1H), 4.01-3.85 (m, 1H), 2.64 (d, J=2.0 Hz, 3H), 2.00-1.77 (m, 3H), 1.76-1.63 (m, 1H), 1.59-1.41 (m, 2H), 0.85-0.69 (m, 2H), 0.69-0.56 (m, 2H), 0.04 (d, J=9.3 Hz, 6H).

### Example 193: MPL-452

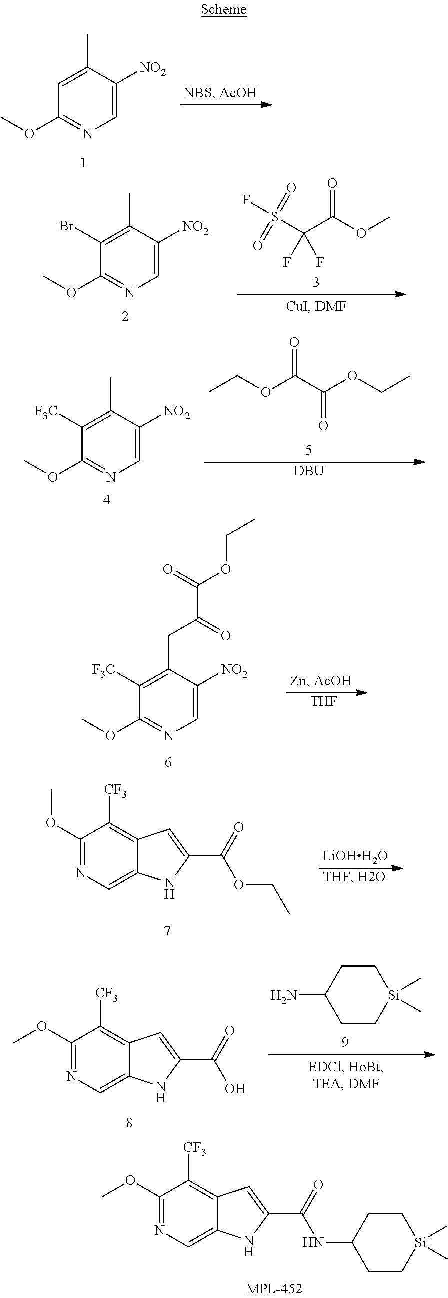

### Synthesis of 3-bromo-2-methoxy-4-methyl-5-nitro-pyridine

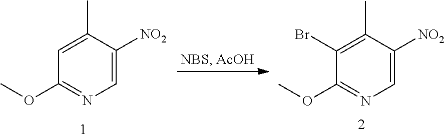

To a solution of 2-methoxy-4-methyl-5-nitro-pyridine (19.61 g, 116.62 mmol, 1 eq) in AcOH (200 mL) was added NBS (83.03 g, 466.49 mmol, 4 eq) under N₂ atmosphere. The suspension was degassed and purged with N₂ for 3 times, and then stirred under N₂ at 110° C. for 18 hr. LCMS showed desired mass. The reaction mixture was poured into ice water (1200 mL), then filtered. The cake was collected and dried under reduced pressure. Compound 3-bromo-2-methoxy-4-methyl-5-nitro-pyridine (18 g, crude) was obtained as a yellow solid.

LCMS (ESI) m/z: 248.1 [M+H]⁺; ¹H NMR was recorded.

### Synthesis of 2-methoxy-4-methyl-5-nitro-3-(trifluoromethyl) pyridine

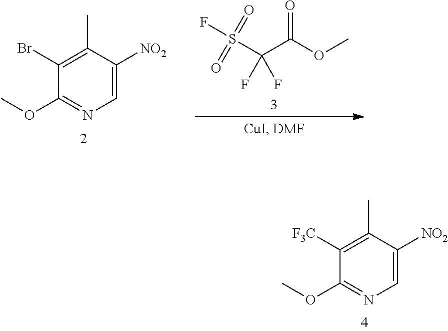 [SMILES: COc1ncc([N+](=O)[O-])c(C)c1Br ; COC(=O)C(F)(F)S(=O)(=O)F ; COc1ncc([N+](=O)[O-])c(C)c1C(F)(F)F]

To a solution of 3-bromo-2-methoxy-4-methyl-5-nitro-pyridine (17 g, 68.81 mmol, 1 eq) in DMF (200 mL) was added CuI (52.42 g, 275.25 mmol, 4 eq) and methyl 2,2-difluoro-2-fluorosulfonyl-acetate (85.00 g, 442.47 mmol, 56.29 mL, 6.43 eq). The mixture was stirred at 100° C. for 5 hr. LCMS showed desired mass. The reaction was poured in saturated NaHCO₃ (500 mL), and then extracted with EtOAC (120 mL×3). The combined organic layer was washed with brine (100 mL×2), dried over Na₂SO₄, and then filtered and concentrated under reduced pressure. The resulting residue was purified by column chromatography (SiO₂, 0-2% ethyl acetate in petroleum ether). Compound 2-methoxy-4-methyl-5-nitro-3-(trifluoromethyl) pyridine (15 g, 47.64 mmol, 69.23% yield, 75% purity) was obtained as a yellow oil.

LCMS (ESI) m/z: 238.1 [M+H]⁺; ¹H NMR (400 MHz, DMSO) was recorded.

### Synthesis of ethyl 3-[2-methoxy-5-nitro-3-(trifluoromethyl)-4-pyridyl]-2-oxo-propanoate

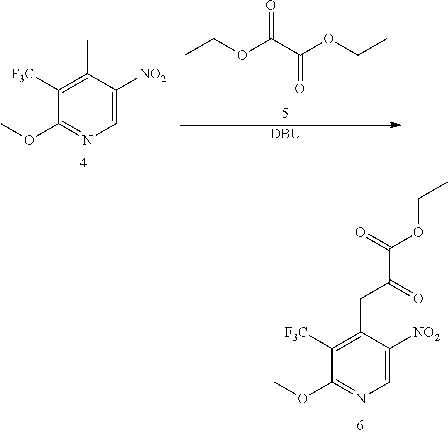

To a solution of 2-methoxy-4-methyl-5-nitro-3-(trifluoromethyl)pyridine (1 g, 4.23 mmol, 1 eq) in diethyl oxalate (10.70 g, 73.22 mmol, 10 mL, 17.29 eq) was added DBU (2.58 g, 16.94 mmol, 2.55 mL, 4 eq). The mixture was stirred at 40° C. for 12 hr. LCMS showed desired mass. The residue was diluted with water (50 mL) and extracted with EtOAc (50 mL×3). The combined organic layer was washed with brine (50 mL×2), dried over Na₂SO₄, and filtered and concentrated under reduced pressure. The residue was purified by column chromatography (SiO₂, 0-10% ethyl acetate in petroleum ether). Compound ethyl 3-[2-methoxy-5-nitro-3-(trifluoromethyl)-4-pyridyl]-2-oxo-propanoate (1.22 g, crude) was obtained as a yellow oil, which was used for the next step without further purification.

LCMS (ESI) m/z: 337.1 [M+H]⁺; ¹H NMR was recorded.

### Synthesis of ethyl 5-methoxy-4-(trifluoromethyl)-1H-pyrrolo[2,3-c]pyridine-2-carboxylate

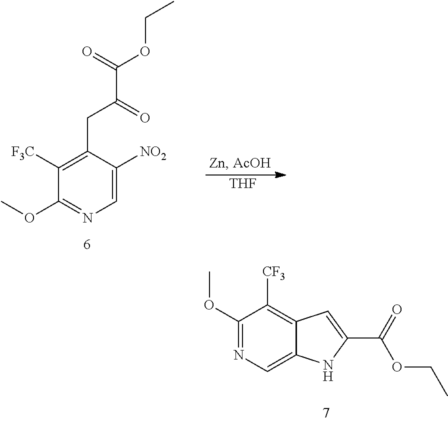 [SMILES: CCOC(=O)C(=O)Cc1c([N+](=O)[O-])cnc(OC)c1C(F)(F)F ; "THF" ; CCOC(=O)c1cc2c(C(F)(F)F)c(OC)ncc2[nH]1]

To a solution of ethyl 3-[2-methoxy-5-nitro-3-(trifluoromethyl)-4-pyridyl]-2-oxo-propanoate (1.22 g, 3.63 mmol, 1 eq) in THF (10 mL) was added Zn (2.37 g, 36.29 mmol, 10 eq) and AcOH (1.09 g, 18.14 mmol, 1.04 mL, 5 eq). The mixture was stirred at 70° C. for 4 hr. LCMS showed desired mass. The reaction mixture was filtered. The filtrate was purified by column chromatography (SiO₂, 0-33% ethyl acetate in petroleum ether). Compound Ethyl 5-methoxy-4-(trifluoromethyl)-1H-pyrrolo[2,3-c]pyridine-2-carboxylate (500 mg, 520.43 umol, 14.34% yield, 30% purity) was obtained as a white solid.

LCMS (ESI) m/z: 289.1 [M+H]⁺; ¹H NMR was recorded.

### Synthesis of 5-methoxy-4-(trifluoromethyl)-1H-pyrrolo[2,3-c]pyridine-2-carboxylic acid

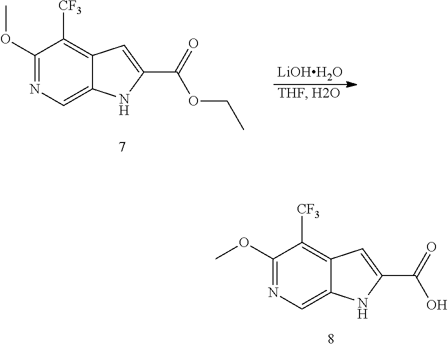

To a solution of ethyl 5-methoxy-4-(trifluoromethyl)-1H-pyrrolo[2,3-c]pyridine-2-carboxylate (500 mg, 871.03 umol, 50.21% purity, 1 eq) in THF (3 mL) was added a solution of LiOH.H₂O (219.31 mg, 5.23 mmol, 6 eq) in H₂O (3 mL). The mixture was stirred at 80° C. for 2 hr. LC-MS showed desired mass. The reaction mixture was concentrated under reduced pressure to remove THE (3 mL). The aqueous phase was adjusted to pH to 3-4 with aqueous HCl (6 N) and then purified by prep-HPLC (column: Phenomenex Synergi C18 150*30 mm*4 um; mobile phase: A: 0.225% formic acid in water, B: CH₃CN, gradient; 32%-62% B over 11 min). Compound 5-methoxy-4-(trifluoromethyl)-1H-pyrrolo[2,3-c]pyridine-2-carboxylic acid (53 mg, 193.53 umol, 22.22% yield, 95% purity) was obtained as a brown solid.

LCMS (ESI) m/z: 261.2 [M+H]⁺; ¹H NMR was recorded.

### Synthesis of N-(1,1-dimethylsilinan-4-yl)-5-methoxy-4-(trifluoromethyl)-1H-pyrrolo[2,3-c]pyridine-2-carboxamide

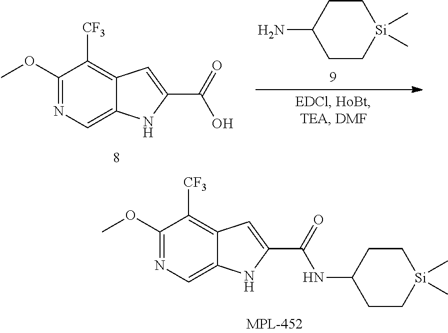

To a solution of 5-methoxy-4-(trifluoromethyl)-1H-pyrrolo[2,3-c]pyridine-2-carboxylic acid (50 mg, 192.18 umol, 1 eq) and 1,1-dimethylsilinan-4-amine (41.46 mg, 230.62 umol, 1.2 eq, HCl salt) in DMF (2 mL) was added a solution of EDCI (110.53 mg, 576.55 umol, 3 eq) and HOBt (77.90 mg, 576.55 umol, 3 eq) in DMF (1 mL), followed by TEA (97.23 mg, 960.91 umol, 133.75 uL, 5 eq). The mixture was stirred at 25° C. for 1 hr. LCMS showed desired mass. The reaction mixture was filtered. The filtrate was purified by prep-HPLC (column: Phenomenex Synergi C18 150*30 mm*4 um; mobile phase: A: 0.225% formic acid in water, B: CH₃CN, gradient: 63%-93% B over 11 min). Compound N-(1,1-dimethylsilinan-4-yl)-5-methoxy-4-(trifluoromethyl)-1H-pyrrolo[2,3-c]pyridine-2-carboxamide (25.6 mg, 66.26 umol, 34.48% yield, 99.8% purity) was obtained as a white solid.

LCMS m/z: 386.1 [M+1]⁺; ¹H NMR (500 MHz, DMSO-d6) δ=12.27 (s, 1H), 8.69-8.53 (m, 2H), 7.26 (d, J=1.7 Hz, 1H), 3.96 (s, 3H), 3.77-3.68 (m, 1H), 2.05-1.94 (m, 2H), 1.67-1.52 (m, 2H), 0.78 (br d, J=14.5 Hz, 2H), 0.62 (dt, J=4.8, 14.2 Hz, 2H), 0.09 (s, 3H), 0.03 (s, 3H).

### Example 194. MPL-352

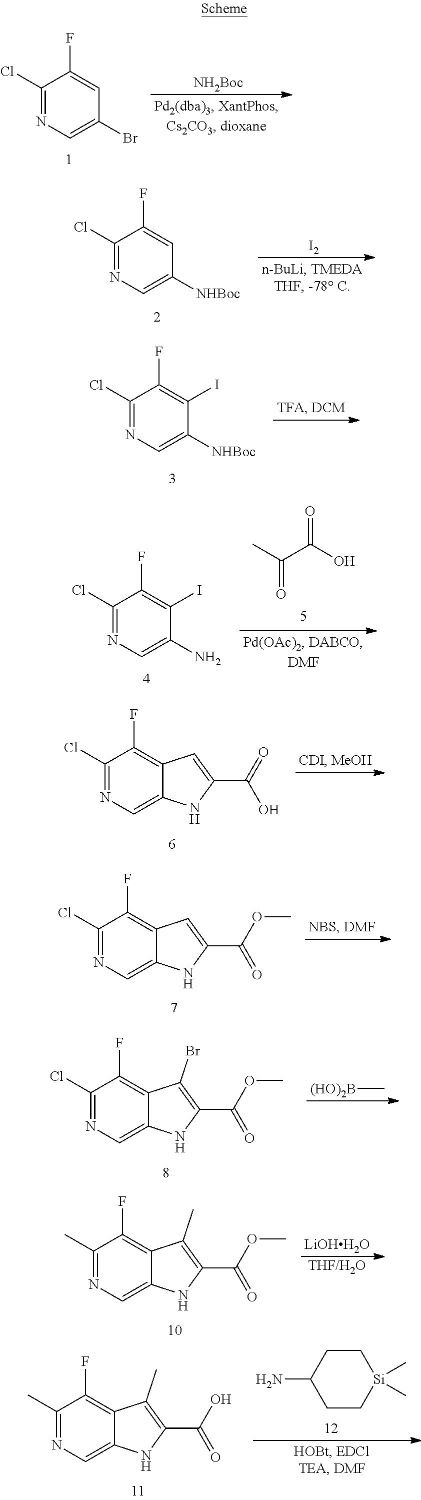

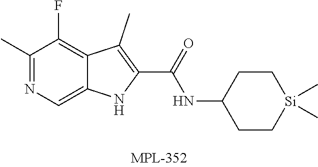

### Step 1. Synthesis of tert-butyl N-(6-chloro-5-fluoro-3-pyridyl)carbamate

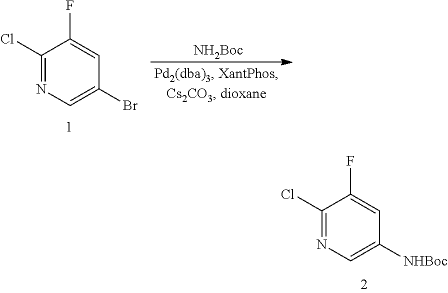 [SMILES: Fc1cc(Br)cnc1Cl ; CC(C)(C)OC(=O)Nc1cnc(Cl)c(F)c1 ; "NH2Boc"]

A mixture of 5-bromo-2-chloro-3-fluoro-pyridine (10 g, 47.52 mmol, 1 eq), tert-butyl carbamate (6.68 g, 57.03 mmol, 1.2 eq), Xantphos (824.90 mg, 1.43 mmol, 0.03 eq) and Cs₂CO₃ (30.97 g, 95.04 mmol, 2 eq) in dioxane (15 mL) was degassed and purged with N₂ for 3 times, then Pd₂(dba)₃ (1.31 g, 1.43 mmol, 0.03 eq) was added. The reaction mixture was stirred at 85° C. for 12 hr under N₂ atmosphere. LCMS indicated desired mass. The mixture was filtered. The filtrate was purified by column chromatography (SiO₂, 0-20% ethyl acetate in petroleum ether). Compound tert-butyl N-(6-chloro-5-fluoro-3-pyridyl)carbamate (6.5 g, 25.03 mmol, 47.89% yield, 95% purity) was obtained as a pale-orange solid.

LCMS (ESI) m/z: 247.0 [M+H]⁺; ¹H NMR was recorded.

### Synthesis of tert-butyl N-(6-chloro-5-fluoro-4-iodo-3-pyridyl)carbamate

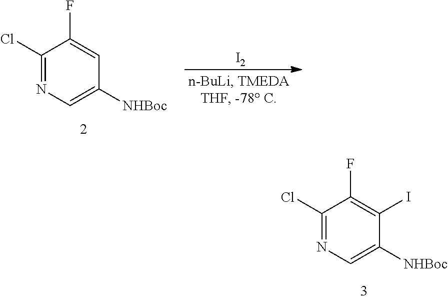 [SMILES: CC(C)(C)OC(=O)Nc1cnc(Cl)c(F)c1 ; "I2" ; CC(C)(C)OC(=O)Nc1cnc(Cl)c(F)c1I]

To a solution of tert-butyl N-(6-chloro-5-fluoro-3-pyridyl)carbamate (1 g, 4.05 mmol, 1 eq) and TMEDA (942.21 mg, 8.11 mmol, 1.22 mL, 2 eq) in THF (12 mL) was added n-BuLi (2.5 M in n-hexane, 4.05 mL, 2.5 eq) dropwise at −78° C. under N₂. After stirring at −78° C. for 30 min, a solution of I₂ (1.54 g, 6.08 mmol, 1.22 mL, 1.5 eq) in THF (5 mL) was added dropwise at −78° C. The reaction mixture was stirred at −78° C. for another 30 min. TLC (petroleum ether:ethyl acetate=3:1) indicated compound 2 was consumed completely and one new spot formed. The reaction mixture was quenched with saturated Na₂SO₃ (20 mL) at 25° C., and then diluted with H₂O (10 mL) and extracted with EtOAc (30 mL×2). The combined organic layer was washed with brine (20 mL), dried over Na₂SO₄, and then filtered and concentrated under reduced pressure. The residue was purified by column chromatography (SiO₂, 0-10% ethyl acetate in petroleum ether). Compound tert-butyl N-(6-chloro-5-fluoro-4-iodo-3-pyridyl)carbamate (1.27 g, 3.24 mmol, 79.88% yield, 95% purity) was obtained as a white solid. ¹H NMR was recorded.

### Synthesis of 6-chloro-5-fluoro-4-iodo-pyridin-3-amine

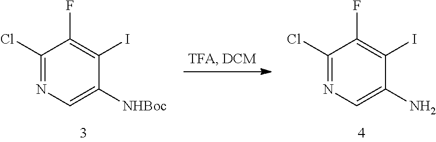 [SMILES: CC(C)(C)OC(=O)Nc1cnc(Cl)c(F)c1I ; Nc1cnc(Cl)c(F)c1I]

To a solution of tert-butyl N-(6-chloro-5-fluoro-4-iodo-3-pyridyl)carbamate (8.68 g, 23.30 mmol, 1 eq) in DCM (10 mL) was added TFA (47.74 g, 418.69 mmol, 31.00 mL, 17.97 eq). The mixture was stirred at 30° C. for 12 hr. LCMS showed desired mass. The reaction mixture was concentrated under reduced pressure to remove solvent. The residue was dissolved in saturated NaHCO₃ (5 mL), and then extracted with ethyl acetate (15 mL×2). The combined organic layer was washed with brine (15 mL), dried over Na₂SO₄, filtered and concentrated under reduced pressure. The residue was purified by column chromatography (SiO₂, 0-20% ethyl acetate in petroleum ether). Compound 6-chloro-5-fluoro-4-iodo-pyridin-3-amine (6.26 g, 21.83 mmol, 81.47% yield, 95% purity) was obtained as a white solid.

LCMS (ESI) m/z: 272.9 [M+H]⁺; ¹H NMR was recorded.

### Synthesis of 5-chloro-4-fluoro-1H-pyrrolo[2,3-c]pyridine-2-carboxylic acid

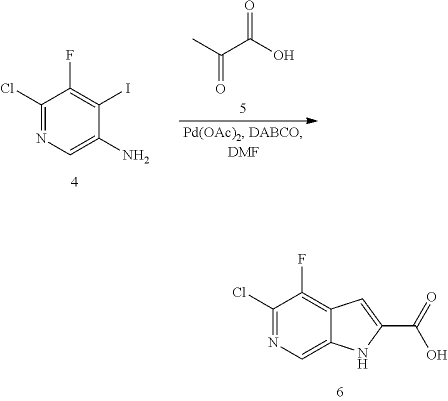

A mixture of 6-chloro-5-fluoro-4-iodo-pyridin-3-amine (6.26 g, 22.98 mmol, 1 eq), 2-oxopropanoic acid (4.05 g, 45.95 mmol, 3.24 mL, 2 eq) and DABCO (5.15 g, 45.95 mmol, 5.05 mL, 2 eq) in DMF (50 mL) was degassed and purged with N₂ for 3 times, Pd(OAc)₂ (515.85 mg, 2.30 mmol, 0.1 eq) was then added. The mixture was stirred at 110° C. for 4 hr under N₂ atmosphere. LC-MS showed desired mass. The reaction mixture was concentrated under reduced pressure to remove DMF. The residue was diluted with toluene (50 mL), sonicated for 30 minutes and filtered. The filter cake was suspended in CH₃CN and filtered. The cake was collected and dried under reduced pressure. Compound 5-chloro-4-fluoro-1H-pyrrolo[2,3-c]pyridine-2-carboxylic acid (5 g, crude) was obtained as a brown solid, which was used for the next step without further purification.

LCMS (ESI) m/z: 215.0 [M+H]⁺; ¹H NMR was recorded.

### Synthesis of methyl 5-chloro-4-fluoro-1H-pyrrolo[2,3-c]pyridine-2-carboxylate

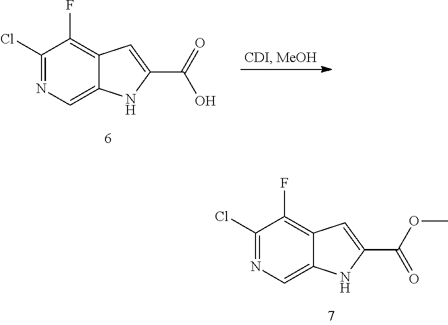

A solution of 5-chloro-4-fluoro-1H-pyrrolo[2,3-c]pyridine-2-carboxylic acid (1.29 g, 6.01 mmol, 1 eq) and CDI (1.07 g, 6.61 mmol, 1.1 eq) in DMF (10 mL) was stirred at 30° C. for 1 hr. MeOH (9.50 g, 296.54 mmol, 12 mL, 49.33 eq) was then added. The mixture was stirred at 30° C. for 1 hr. LCMS showed desired mass. The reaction mixture was concentrated under reduced pressure to remove MeOH and then poured into H₂O (100 mL), the suspension was filtered. The aqueous filtrate was extracted with a mixed solvent of dichloromethane and methanol (10:1) (50 mL×3). The solid was then dissolved in the combined organic phase, which was dried over Na₂SO₄, filtered and concentrated under reduced pressure. Compound methyl 5-chloro-4-fluoro-1H-pyrrolo[2,3-c]pyridine-2-carboxylate (1.1 g, 3.85 mmol, 64.03% yield, 80% purity) was obtained as a brown solid. The crude product was used for the next step without further purification.

LCMS (ESI) m/z: 229.0 [M+H]⁺; ¹H NMR was recorded.

### Synthesis of Compound methyl 3-bromo-5-chloro-4-fluoro-1H-pyrrolo[2,3-c]pyridine-2-carboxylate

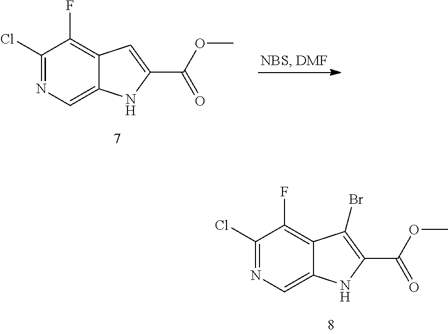 [SMILES: COC(=O)c1cc2c(F)c(Cl)ncc2[nH]1 ; COC(=O)c1[nH]c2cnc(Cl)c(F)c2c1Br]

A mixture of methyl 5-chloro-4-fluoro-1H-pyrrolo[2,3-c]pyridine-2-carboxylate (500 mg, 2.19 mmol, 1 eq) and NBS (428.21 mg, 2.41 mmol, 1.1 eq) in DMF (10 mL) under N₂ was stirred at 30° C. for 3 hr. LC-MS showed desired mass. The mixture was poured into water (100 mL) and the suspension was filtered. The filter cake was washed with water (50 mL), collected and dried in vacuo. Compound methyl 3-bromo-5-chloro-4-fluoro-1H-pyrrolo[2,3-c]pyridine-2-carboxylate (459 mg, 1.34 mmol, 61.42% yield, 90% purity) was obtained as a brown solid. The crude product was used for the next step without further purification.

LCMS (ESI) m/z: 309.0 [M+H]⁺; H NMR was recorded.

### Synthesis of methyl 4-fluoro-3,5-dimethyl-1H-pyrrolo[2,3-c]pyridine-2-carboxylate

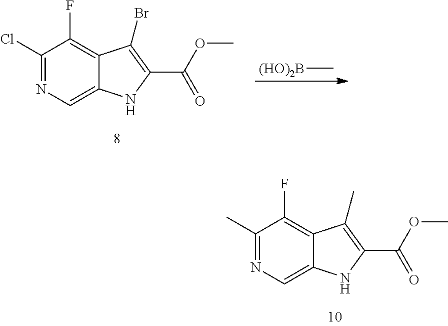

A mixture of methyl 3-bromo-5-chloro-4-fluoro-1H-pyrrolo[2,3-c]pyridine-2-carboxylate (600 mg, 1.95 mmol, 1 eq), methylboronic acid (583.99 mg, 9.76 mmol, 5 eq), K₃PO₄ (1.24 g, 5.85 mmol, 3 eq) and XPhos (186.04 mg, 390.24 umol, 0.2 eq) in dioxane (4 mL) was de-gassed under N₂ atmosphere. Pd₂(dba)₃ (357.35 mg, 390.24 umol, 0.2 eq) was then added. The suspension was degassed and purged with N₂ for 3 times, and stirred under N₂ at 120° C. for 12 hr. LCMS showed desired mass. EtOAc (30 mL) was added. The mixture was filtered to remove the insoluble materials. The filtrate was concentrated in vacuo. The residue was purified by column chromatography (SiO₂, 0-100% ethyl acetate in petroleum ether). Compound methyl 4-fluoro-3,5-dimethyl-1H-pyrrolo[2,3-c]pyridine-2-carboxylate (170 mg, 650.27 umol, 28.73% yield, 85% purity) was obtained as a yellow solid.

LCMS (ESI) m/z: 223.1 [M+H]⁺; ¹H NMR was recorded.

### Synthesis of 4-fluoro-3,5-dimethyl-1H-pyrrolo[2,3-c]pyridine-2-carboxylic acid

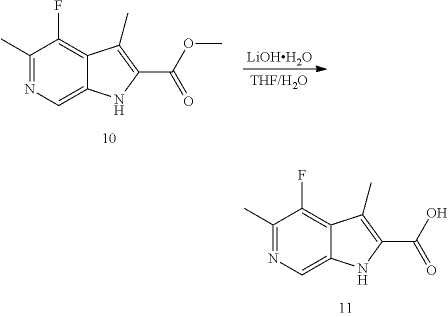

To a solution of methyl 4-fluoro-3,5-dimethyl-1H-pyrrolo[2,3-c]pyridine-2-carboxylate (170 mg, 765.02 umol, 1 eq) in THE (2 mL) was added a solution of LiOH.H₂O (192.62 mg, 4.59 mmol, 6 eq) in H₂O (2 mL). The mixture was stirred at 80° C. for 12 hr. LC-MS showed desired mass. The reaction mixture was concentrated under reduced pressure to remove THE (2 mL). The aqueous solution was adjusted to pH to 3-4 with aqueous HCl (1 N) and then filtered. The cake was collected, washed with petroleum ether (15 mL), and dried under reduced pressure. Compound 4-fluoro-3,5-dimethyl-1H-pyrrolo[2,3-c]pyridine-2-carboxylic acid (102 mg, 440.95 umol, 57.64% yield, 90% purity) was obtained as a yellow solid, which was used for the next step without further purification.

LCMS (ESI) m/z: 209.1 [M+H]⁺; ¹H NMR (500 MHz, DMSO-d₆) δ=12.01 (s, 1H), 8.48 (s, 1H), 2.62 (s, 3H), 2.44 (d, J=3 Hz, 3H).

### Synthesis of N-(1,1-dimethylsilinan-4-yl)-4-fluoro-3,5-dimethyl-1H-pyrrolo[2,3-c]pyridine-2-carboxamide

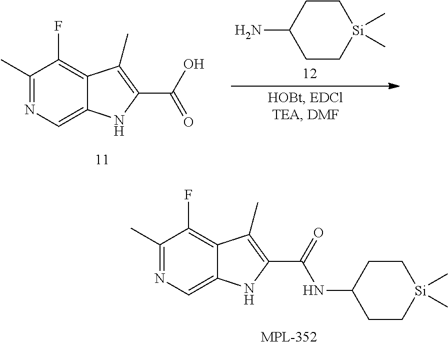

To a solution of 4-fluoro-3,5-dimethyl-1H-pyrrolo[2,3-c]pyridine-2-carboxylic acid (50 mg, 240.17 umol, 1 eq) and 1,1-dimethylsilinan-4-amine (51.81 mg, 288.20 umol, 1.2 eq, HCl salt) in DMF (1.5 mL) was added a solution of EDCI (138.12 mg, 720.50 umol, 3 eq) and HOBt (97.36 mg, 720.50 umol, 3 eq) in DMF (0.5 mL), followed by TEA (145.81 mg, 1.44 mmol, 200.57 uL, 6 eq). The mixture was stirred at 25° C. for 1 hr. LCMS showed desired mass. The reaction mixture was filtered. The residue was purified by prep-HPLC (column: Agela DuraShell C18 150*25 mm*5 um; mobile phase: A: 0.04% NH₃H₂O and 10 mM of NH₄HCO₃ in water, B: CH₃CN; gradient: 49%-79% B over 10 min). Compound N-(1,1-dimethylsilinan-4-yl)-4-fluoro-3,5-dimethyl-1H-pyrrolo[2,3-c]pyridine-2-carboxamide (24.7 mg, 74.07 umol, 30.84% yield, 100% purity) was obtained as a white solid.

LCMS (ESI) m/z: 334.1 [M+H]⁺; ¹H NMR (500 MHz, DMSO-d₆) δ=8.47 (d, J=2.3 Hz, 1H), 8.00 (d, J=7.9 Hz, 1H), 3.77-3.67 (m, 1H), 2.54 (s, 3H), 2.44 (d, J=3.2 Hz, 3H), 2.07-1.96 (m, 2H), 1.70-1.53 (m, 2H), 0.83-0.71 (m, 2H), 0.61 (dt, J=4.7, 13.8 Hz, 2H), 0.10-0.00 (m, 6H).

### Example 195. MPL-353

### Synthesis of N-(1,1-dimethylsilepan-4-yl)-4-fluoro-3,5-dimethyl-1H-pyrrolo[2,3-c]pyridine-2-carboxamide

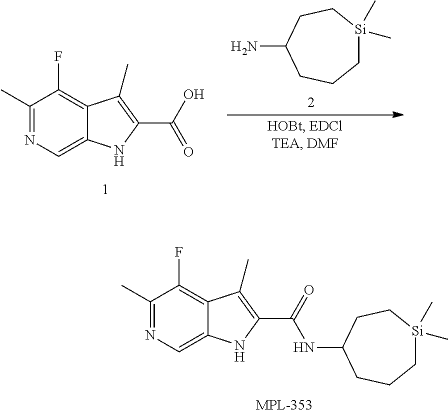

To a solution of 4-fluoro-3,5-dimethyl-1H-pyrrolo[2,3-c]pyridine-2-carboxylic acid (50 mg, 240.17 umol, 1 eq) and 1,1-dimethylsilepan-4-amine (55.85 mg, 288.20 umol, 1.2 eq, HCl salt) in DMF (1.5 mL) was added a solution of EDCI (138.12 mg, 720.51 umol, 3 eq) and HOBt (97.36 mg, 720.51 umol, 3 eq) in DMF (0.5 mL), followed by TEA (145.82 mg, 1.44 mmol, 200.57 uL, 6 eq). The mixture was stirred at 25° C. for 1 hr. LCMS indicated desired mass. The reaction mixture was filtered. The filtrate was purified by prep-HPLC (column, Agela DuraShell C18 150*25 mm*5 um; mobile phase: A: 0.04% NH₃H₂O and 10 mM of NH₄HCO₃ in water, B: CH₃CN; gradient: 51%-81% B over 10 min). Compound N-(1,1-dimethylsilepan-4-yl)-4-fluoro-3,5-dimethyl-1H-pyrrolo[2,3-c]pyridine-2-carboxamide (23.3 mg, 66.92 umol, 27.86% yield, 99.806% purity) was obtained as a white solid.

LCMS m/z: 348.1 [M+1]⁺; ¹H NMR (500 MHz, DMSO-d₆) δ=11.79 (br s, 1H), 8.47 (d, J=2.3 Hz, 1H), 8.06 (d, J=7.6 Hz, 1H), 3.93-3.84 (m, 1H), 2.54 (s, 3H), 2.44 (d, J=3.4 Hz, 3H), 2.01-1.75 (m, 3H), 1.68 (dq, J=2.0, 11.5 Hz, 1H), 1.56-1.41 (m, 2H), 0.84-0.69 (m, 2H), 0.67-0.53 (m, 2H), 0.03 (d, J=3.4 Hz, 6H).

### Example 196. MPL-464

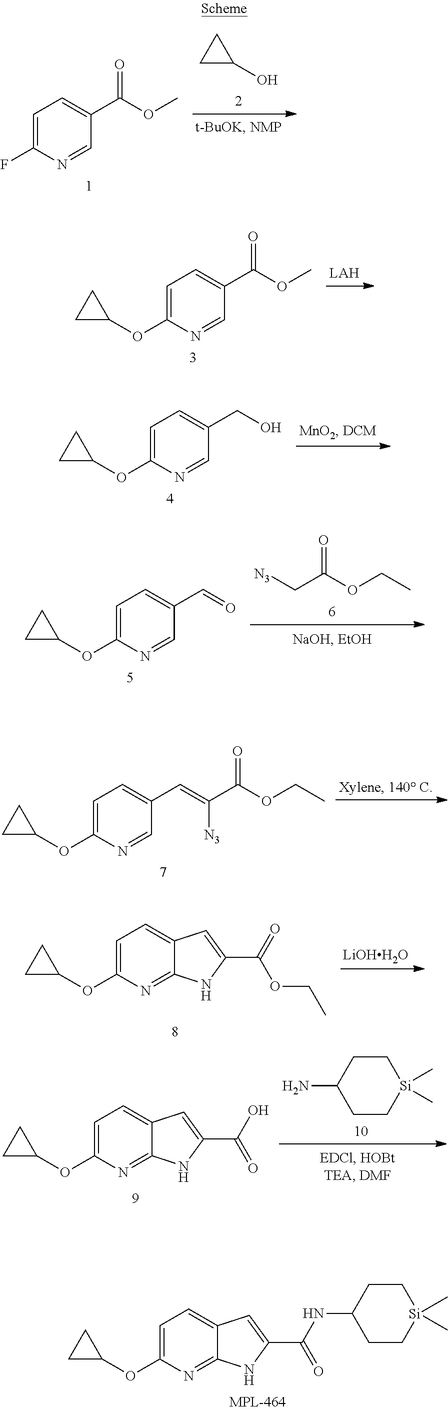

### Synthesis of methyl 6-(cyclopropoxy) pyridine-3-carboxylate

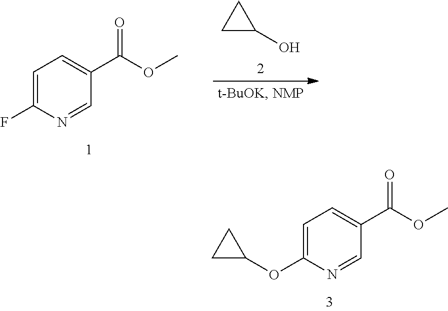

To a solution of methyl 6-fluoropyridine-3-carboxylate (500 mg, 3.22 mmol, 1 eq) in NMP (10 mL) was added cyclopropanol (224.64 mg, 3.87 mmol, 1.2 eq). The mixture was stirred at 0° C. for 5 min. t-BuOK (723.36 mg, 6.45 mmol, 2 eq) was then added dropwise at 0° C. The mixture was stirred at 25° C. for 12 hr. LC-MS showed desired mass. The reaction mixture was poured into a mixed solvent of petroleum ether/Ethyl acetate/H₂O (20 mL/20 mL/40 mL). The organic layer was washed with 5% of aqueous solution LiCl (20 mL), dried over anhydrous Na₂SO₄, and filtered. The filtrate was concentrated in vacuo. The residue was purified by column chromatography (SiO₂, 0-20% Ethyl acetate in petroleum ether). Compound methyl 6-(cyclopropoxy) pyridine-3-carboxylate (250 mg, 1.04 mmol, 32.12% yield, 80% purity) was obtained as a white solid.

LCMS (ESI) m/z: 194.1 [M+H]⁺; ¹H NMR was recorded.

### Synthesis of [6-(cyclopropoxy)-3-pyridyl]methanol

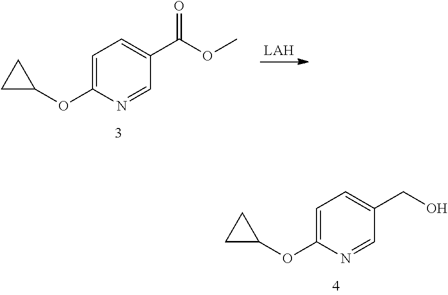

To an ice-cooled solution of methyl 6-(cyclopropoxy) pyridine-3-carboxylate (1.3 g, 6.73 mmol, 1 eq) in dried THE (12 mL) was added LAH (383.08 mg, 10.09 mmol, 1.5 eq) in batches. The mixture was stirred at 0° C. for 1 hr. TLC (Petroleum ether:Ethyl acetate=10:1) indicated compound 3 was consumed completely and one new spot formed. The reaction was quenched with water (0.383 mL), NaOH (15%, 0.383 mL) and water (1.149 mL). The mixture was dried over Na₂SO₄, and then filtered and concentrated under reduced pressure. The residue was purified by column chromatography (SiO₂, 0-100% Ethyl acetate in petroleum ether). Compound [6-(cyclopropoxy)-3-pyridyl]methanol (800 mg, 3.87 mmol, 57.58% yield, 80% purity) was obtained as a brown oil. ¹H NMR was recorded.

### Synthesis of 6-(cyclopropoxy) pyridine-3-carbaldehyde

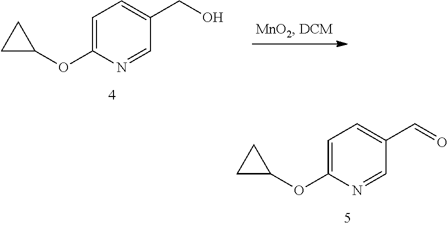 [SMILES: OCc1ccc(OC2CC2)nc1 ; O=Cc1ccc(OC2CC2)nc1]

To a solution of [6-(cyclopropoxy)-3-pyridyl]methanol (800 mg, 4.84 mmol, 1 eq) in DCM (10 mL) was added MnO₂ (4.21 g, 48.43 mmol, 10 eq). The mixture was stirred at 25° C. for 12 hr. TLC (Petroleum ether:Ethyl acetate=5:1) indicated compound 4 was consumed completely, and a new spot was detected. The reaction mixture was filtered and concentrated under reduced pressure. The resulting residue was purified by column chromatography (SiO₂, 0-20% Ethyl acetate in petroleum ether). Compound 6-(cyclopropoxy) pyridine-3-carbaldehyde (733 mg, 3.59 mmol, 74.21% yield, 80% purity) was obtained as a colorless oil. ¹H NMR was recorded.

### Step 4. Synthesis of ethyl (Z)-2-azido-3-[6-(cyclopropoxy)-3-pyridyl]prop-2-enoate

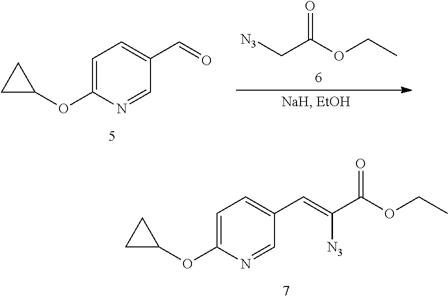

NaH (539.01 mg, 13.48 mmol, 60% purity, 3 eq) was added to EtOH (10 mL) in batches. The mixture was stirred at 20° C. to a clear solution then cooled to −10° C. Then a solution of 6-(cyclopropoxy) pyridine-3-carbaldehyde (733 mg, 4.49 mmol, 1 eq) and ethyl 2-azidoacetate (1.74 g, 13.48 mmol, 1.89 mL, 3 eq) in THE (10 mL) was added dropwise. The reaction mixture was stirred at −10° C.˜0° C. for 2 hr. TLC (Petroleum ether:Ethyl acetate=5:1) indicated many new spots formed and compound 5 was also detected. The reaction was quenched with saturated NH₄Cl (60 mL), and then extracted with EtOAc (50 mL×2). The combined organic layer was washed with brine (60 mL×2), dried over Na₂SO₄, and then filtered and concentrated under reduced pressure. The resulting residue was purified by column chromatography (SiO₂, 0-6% Ethyl acetate in petroleum ether). Compound ethyl (Z)-2-azido-3-[6-(cyclopropoxy)-3-pyridyl]prop-2-enoate (506 mg, crude) was obtained as a yellow oil.

LCMS (ESI) m/z: 275.1 [M+H]⁺

### Synthesis of ethyl 6-(cyclopropoxy)-1H-pyrrolo[2,3-b]pyridine-2-carboxylate

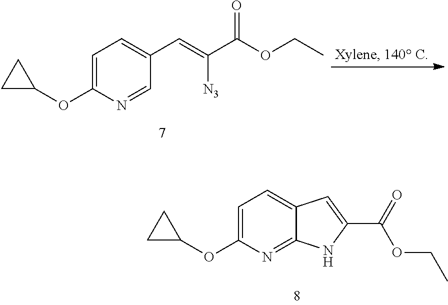 [SMILES: CCOC(=O)/C(=C/c1ccc(OC2CC2)nc1)N=[N+]=[N-] ; CCOC(=O)c1cc2ccc(OC3CC3)nc2[nH]1]

A solution of ethyl (Z)-2-azido-3-[6-(cyclopropoxy)-3-pyridyl]prop-2-enoate (500 mg, 1.82 mmol, 1 eq) in xylene (5 mL) was stirred at 140° C. for 30 min. LC-MS showed desired mass. The reaction mixture was filtered and concentrated under reduced pressure. The residue was purified by column chromatography (SiO₂, 0-25% Ethyl acetate in petroleum ether). Compound ethyl 6-(cyclopropoxy)-1H-pyrrolo[2,3-b]pyridine-2-carboxylate (396 mg, 1.45 mmol, 79.39% yield, 90% purity) was obtained as a colorless oil.

LCMS (ESI) m/z: 247.1 [M+H]⁺; ¹H NMR was recorded.

### Synthesis of 6-(cyclopropoxy)-1H-pyrrolo[2,3-b]pyridine-2-carboxylic acid

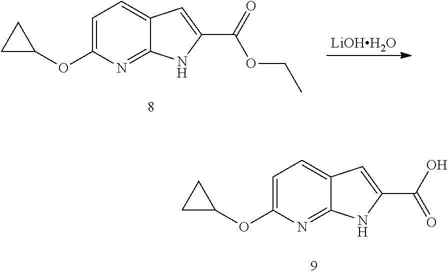

To a solution of ethyl 6-(cyclopropoxy)-1H-pyrrolo[2,3-b]pyridine-2-carboxylate (340 mg, 1.38 mmol, 1 eq) in THE (1.5 mL) was added a solution of LiOH.H₂O (347.62 mg, 8.28 mmol, 6 eq) in H₂O (1.5 mL). The mixture was stirred at 80° C. for 6 hr. TLC (Petroleum ether:Ethyl acetate=5:1) indicated compound 8 was consumed completely and one new spot formed. The reaction mixture was concentrated under reduced pressure to remove THF. The aqueous solution was adjusted to pH to 3-4 with aqueous HCl (1 N), and then filtered. The cake was washed with petroleum ether (15 mL), and then dried under reduced pressure. Compound 6-(cyclopropoxy)-1H-pyrrolo[2,3-b]pyridine-2-carboxylic acid (299 mg, 1.16 mmol, 84.36% yield, 85% purity) was obtained as a white solid.

¹H NMR (500 MHz, DMSO-d₆) δ=12.79 (br s, 1H), 12.29-11.95 (m, 1H), 8.02-7.91 (m, 1H), 7.07-6.99 (m, 1H), 6.61 (d, J=8.5 Hz, 1H), 4.28 (tt, J=3.1, 6.2 Hz, 1H), 0.81-0.66 (m, 4H).

### Synthesis of 6-(cyclopropoxy)-N-(1,1-dimethylsilinan-4-yl)-1H-pyrrolo[2,3-b]pyridine-2-carboxamide

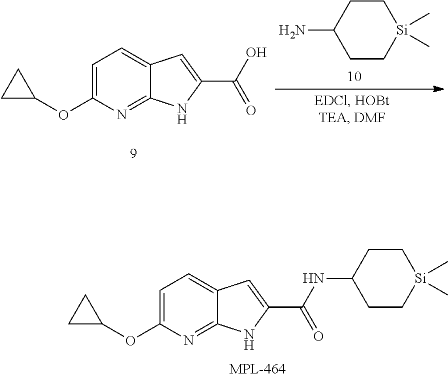

To a solution of 6-(cyclopropoxy)-1H-pyrrolo[2,3-b]pyridine-2-carboxylic acid (60 mg, 274.97 umol, 1 eq) and 1,1-dimethylsilinan-4-amine (59.31 mg, 329.96 umol, 1.2 eq, HCl salt) in DMF (2 mL) was added a solution of EDCI (158.14 mg, 824.90 umol, 3 eq) and HOBt (111.46 mg, 824.90 umol, 3 eq) in DMF (1 mL), followed by TEA (139.12 mg, 1.37 mmol, 191.36 uL, 5 eq). The mixture was stirred at 25° C. for 1 hr. LC-MS showed desired mass. The reaction mixture was filtered. The filtrate was purified by prep-HPLC (column: Phenomenex Synergi C18 150*30 mm*4 um; mobile phase: A: 0.225% formic acid in water, B: CH₃CN; gradient: 60%-70% B over 11 min). Compound 6-(cyclopropoxy)-N-(1,1-dimethylsilinan-4-yl)-1H-pyrrolo[2,3-b]pyridine-2-carboxamide (49.7 mg, 143.01 umol, 52.01% yield, 98.84% purity) was obtained as a white solid.

LCMS (ESI) m/z: 344.1 [M+H]⁺; ¹H NMR (400 MHz, DMSO-d6) δ=11.88 (s, 1H), 8.00-7.90 (m, 2H), 7.01 (d, J=2.0 Hz, 1H), 6.58 (d, J=8.6 Hz, 1H), 4.30-4.19 (m, 1H), 3.70 (br d, J=7.8 Hz, 1H), 1.98 (br d, J=10.6 Hz, 2H), 1.66-1.49 (m, 2H), 0.83-0.54 (m, 8H), 0.11-0.01 (m, 6H).

### Example 197: Biological Experiments

MIC (Minimum Inhibitory Concentration) determination of anti-tuberculosis drugs: The antituberculosis activity of each compound against M tb H37Rv was measured by the green fluorescent protein reporter assay (L. A. Collins, M. N. Torrero, S. G. Franzblau, Antimicrob. Agents Chemother. 1998, 42, 344-347). Briefly, the compound was initially dissolved in dimethylsulfoxide (DMSO), and two fold dilutions were made in DMSO. The same amount of each dilution of compound solution was added to 7H9 broth in microplates. The initial inoculum of 2×10⁵ CFU/ml of Mtb H37Rv-GFP that was grown in Middlebrook 7H9 media was exposed to the compound for 10 days. The fluorescence was measured in a Fluostar Optima microplate fluorometer (BMG Labtech, Germany), and the MIC was defined as the lowest concentration of compounds that inhibited fluorescence by 90% comparing to the fluorescence of bacteria only wells. CFU=colony forming units.

The Table below shows anti-Mycobacterium tuberculosis activity of representative compounds of the invention:

| | M. TB H37Rv: | | M. TB H37Rv: |
|---|---|---|---|
| Compound | MIC-MABA: | Compound | MIC-MABA: |
| Number | MIC (μg/mL) | Number | MIC (μg/mL) |
| MPL-001 | 0.03 | MPL-122 | 3.1 |
| MPL-002 | 0.056 | MPL-124 | 3.1 |
| MPL-003 | 0.01 | MPL-125 | 3.1 |
| MPL-006 | 0.3 | MPL-126 | 3.1 |
| MPL-007 | 0.12 | MPL-127 | 0.54 |
| MPL-008 | 0.051 | MPL-128 | 2.3 |
| MPL-008 | 0.017 | MPL-129 | 0.5 |
| MPL-012 | 0.048 | MPL-130A | 2.7 |
| MPL-013 | 1.2 | MPL-134 | 3.1 |
| MPL-014 | 5.4 | MPL-135 | 1.5 |
| MPL-015 | 0.044 | MPL-138 | 2.9 |
| MPL-016 | 0.38 | MPL-139 | 0.24 |
| MPL-017 | 0.72 | MPL-140 | 1.3 |
| MPL-019 | 9 | MPL-141 | 0.29 |
| MPL-023 | 11 | MPL-157 | 2.6 |
| MPL-027 | 0.61 | MPL-160 | 0.77 |
| MPL-031 | 0.25 | MPL-163 | 0.64 |
| MPL-033 | 0.038 | MPL-164 | 0.35 |
| MPL-034 | 0.25 | MPL-166 | 0.39 |
| MPL-035 | 0.14 | MPL-169 | 0.13 |
| MPL-036 | 0.08 | MPL-170 | 0.1 |
| MPL-037 | 0.014 | MPL-174 | 0.19 |
| MPL-038 | 6.2 | MPL-189 | 0.095 |
| MPL-039 | 0.39 | MPL-190 | 0.24 |
| MPL-040 | 6.2 | MPL-191 | 0.0087 |
| MPL-041 | 8.5 | MPL-192 | 0.0093 |
| MPL-043 | 0.14 | MPL-195 | 0.014 |
| MPL-043 | 0.17 | MPL-196 | 0.0048 |
| MPL-044 | 0.013 | MPL-197 | 0.013 |
| MPL-045 | 0.069 | MPL-199, | 0.046 |
| | | MPL-213 | |
| MPL-062 | 0.19 | MPL-200 | 0.072 |
| MPL-063 | 0.14 | MPL-202 | 0.11 |
| MPL-064 | 0.16 | MPL-207 | 0.0045 |
| MPL-065a | 0.013 | MPL-208 | 0.0045 |
| MPL-066 | 0.012 | MPL-209 | 0.03 |
| MPL-067 | 0.01 | MPL-210 | 0.058 |
| MPL-068 | 0.046 | MPL-215 | 1.4 |
| MPL-069 | 0.0099 | MPL-216 | 0.041 |
| MPL-070 | 0.49 | MPL-218 | 0.037 |
| MPL-071 | 0.049 | MPL-219 | 0.9 |
| MPL-092 | 1.2 | MPL-221 | 0.2 |
| MPL-093 | 1.1 | MPL-222 | 0.19 |
| MPL-094 | 0.049 | MPL-226 | 0.75 |
| MPL-095 | 1.5 | MPL-229 | 0.0058 |
| MPL-096 | 0.47 | MPL-230 | 0.004 |
| MPL-097 | 0.16 | MPL-236 | 0.18 |
| MPL-100 | 0.24 | MPL-237 | 0.047 |
| MPL-106 | 0.16 | MPL-239 | 0.0078 |
| MPL-108 | 0.02 | MPL-253 | 0.056 |
| MPL-109 | 0.022 | MPL-254 | 0.2 |
| MPL-110 | 0.022 | MPL-259 | 0.054 |
| MPL-111 | 0.0095 | MPL-260 | 0.0075 |
| MPL-118 | 0.017 | | |
| MPL-119 | 0.14 | | |
| MPL-232 | 0.03 | MPL-351 | 0.248 |
| MPL-274 | 0.01 | MPL-352 | 0.122 |
| MPL-275 | 0.031 | MPL-353 | 0.124 |
| MPL-276 | 0.015 | MPL-366 | 0.492 |
| MPL-277 | 0.045 | MPL-367 | 0.988 |
| MPL-280 | 0.043 | MPL-368 | 0.49 |
| MPL-281 | 0.0151 | MPL-376 | 0.188 |
| MPL-282 | <0.004 | MPL-379 | 0.332 |
| MPL-282A | 0.051 | MPL-382 | 0.368 |
| MPL-282B | 0.197 | MPL-387 | 0.163 |
| MPL-284 | 0.026 | MPL-388 | 0.012 |
| MPL-285 | 0.016 | MPL-389 | 0.216 |
| MPL-290 | 0.005 | MPL-391 | 0.051 |
| MPL-292 | 0.0271 | MPL-392 | 0.464 |
| MPL-294 | >1.00 | MPL-401 | 0.016 |
| MPL-295 | 0.024 | MPL-401A | 0.319 |
| MPL-295A | 0.22 | MPL-401B | 0.007 |
| MPL-295B | <0.004 | MPL-402 | 0.962 |
| MPL-301 | 0.015 | MPL-434 | 0.061 |
| MPL-305 | 0.015 | MPL-435 | 1.0 |
| MPL-316 | 0.015 | MPL-445 | 0.119 |
| MPL-316A | 0.014 | MPL-451 | 0.99 |
| MPL-316B | 0.25 | MPL-452 | 0.118 |
| MPL-318 | 0.134 | MPL-453 | 0.284 |
| MPL-319 | 0.499 | MPL-454 | 0.458 |
| MPL-320 | 0.25 | MPL-455 | 0.121 |
| MPL-321 | 0.519 | MPL-464 | 0.124 |
| MPL-322 | 0.484 | MPL-465 | 0.008 |
| MPL-328 | 0.061 | MPL-466 | 0.119 |
| MPL-329 | 0.491 | MPL-466A | 0.042 |
| MPL-345 | 0.498 | MPL-466B | >1 |
| MPL-346 | 0.464 | MPL-467 | 0.121 |
| MPL-348 | 0.435 | MPL-468 | 0.023 |
| MPL-349 | 0.068 | MPL-469 | 0.206 |
| MPL-350 | 0.247 | MPL-471 | 0.069 |

The Table below shows anti-Mycobacterium abscessus activity of representative compounds of the invention:

| | Mab_ATCC:MIC | | |
|---|---|---|---|
| Compound | MHII: MIC | Compound | Mab_ATCC:MIC |
| Number | (μg/mL) | Number | MHII: MIC (μg/mL) |
| MPL-012 | 4 | MPL-135 | 8 |
| MPL-034 | 2 | MPL-188 | 8 |
| MPL-044 | 2 | MPL-195 | 2 |
| MPL-045 | 2 | MPL-200 | 16 |
| MPL-067 | 0.25 | MPL-208 | 1 |
| MPL-118 | 1 | MPL-209 | 1 |
| MPL-119 | 0.5 | MPL-229 | 0.12 |
| MPL-124 | 16 | MPL-230 | 16 |
| MPL-127 | 8 | MPL-239 | 16 |
| MPL-232 | 0.38 | MPL-401 | 0.75 |
| MPL-274 | 0.75 | MPL-464 | 1 |
| MPL-295 | 0.28 | MPL-466A | 0.25 |
| MPL-295B | 0.12 | MPL-466B | 8 |
| MPL-316 | 0.16 | MPL-468 | 0.12 |
| MPL-316A | 0.19 | MPL-471 | 1 |
| MPL-387 | 0.5 | MPL-453 | 0.5 |

Key for Tables: MIC: Minimum Inhibitory Concentration; MABA: microplate-based Alamar Blue assay; Mab: Mycobacterium abscessus; ATCC: American Type Culture Collection; and MHII: Mueller-Hinton broth.

It is to be understood that the invention is not limited to the particular embodiments of the invention described above, as variations of the particular embodiments may be made and still fall within the scope of the appended claims.

The invention will be further described, without limitation, by the following numbered paragraphs:

1. A compound of Formula (I) or Formula (II):

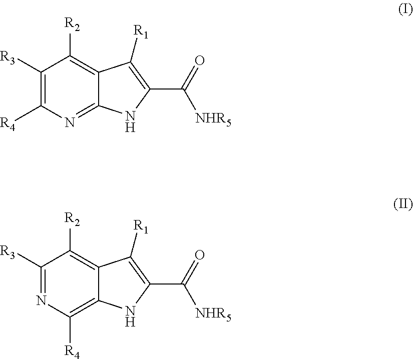

wherein:
R₁ is hydrogen or lower alkyl;
R₂ is hydrogen, lower alkyl, halo, cyano, trifluoromethyl, halo-lower alkyl, di-halo-lower alkyl, alkoxy, or carboxamide;
R₃ is hydrogen, lower alkyl, aryl, heteroaryl, halo, cyano, trifluoromethyl, halo-lower alkyl, di-halo-lower alkyl, alkoxy, or carboxamide;
R₄ is hydrogen, lower alkyl, aryl, heteroaryl, halo, cyano, trifluoromethyl, halo-lower alkyl, di-halo-lower alkyl, alkoxy, cycloalkoxy, or carboxamide;

### R₅ is:

(i) lower alkyl;
(ii) cycloalkyl, cycloalkylene or —CH₂-cycloalkyl;
(iii) spiral(C₈-C₁₁)cycloalkyl;
(iv) phenyl;

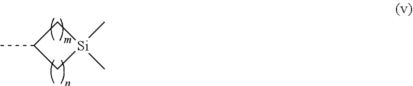

wherein m is 1, 2 or 3 and n is 1, 2, 3, or 4;

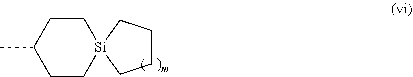

wherein m is 1 or 2; or
(vii) bridged cycloalkyl,
   or a pharmaceutically acceptable salt thereof.
   2. The compound according to paragraph 1, or a pharmaceutically acceptable salt thereof, wherein R₁ is hydrogen or methyl.
   3. The compound according to paragraph 1 or 2, or a pharmaceutically acceptable salt thereof, wherein R₂ is hydrogen, methyl, halo, cyano, trifluoromethyl, mono-fluoromethyl, di-fluoromethyl, methoxy, or carboxamide.
   4. The compound according to any one of paragraphs 1-3, or a pharmaceutically acceptable salt thereof, wherein R₃ is hydrogen, methyl, halo, cyano, trifluoromethyl, mono-fluoromethyl, di-fluoromethyl, methoxy, or carboxamide.
   5. The compound according to any one of paragraphs 1-4, or a pharmaceutically acceptable salt thereof, wherein R₄ is hydrogen, methyl, halo, cyano, trifluoromethyl, mono-fluoromethyl, di-fluoromethyl, methoxy, or carboxamide.
   6. The compound according to any one of paragraphs 1-5, or a pharmaceutically acceptable salt thereof, wherein R₅ is lower alkyl, optionally substituted with phenyl, said phenyl optionally substituted with one or two substituents each independently selected from lower alkyl, halo-substituted lower alkyl, alkoxy, hydroxy lower alkyl, alkoxy lower alkyl, ethynyl, cyano, halo, or hydroxyl;
   7. The compound according to any one of paragraphs 1-5, or a pharmaceutically acceptable salt thereof, wherein R₅ is cycloalkyl, cycloalkylene or —CH₂-cycloalkyl, said cycloalkyl, cycloalkylene or —CH₂-cycloalkyl optionally substituted with one or two substituents each independently selected from lower alkyl, halo-substituted lower alkyl, alkoxy, hydroxyl lower alkyl, alkoxy-lower alkyl, ethynyl, cyano, halo, or hydroxyl.
   8. The compound according to any one of paragraphs 1-5, or a pharmaceutically acceptable salt thereof, wherein R₅ is spiral(C₈-C₁₁)cycloalkyl, optionally substituted with one or two substituents selected from lower alkyl and halogen.
   9. The compound according to any one of paragraphs 1-5, or a pharmaceutically acceptable salt thereof, wherein R₅ is

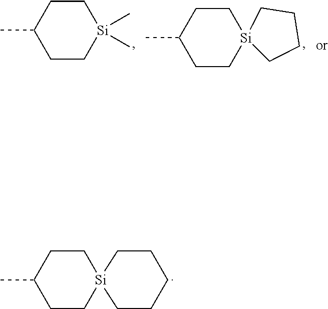

10. The compound according to any one of paragraphs 1-5, or a pharmaceutically acceptable salt thereof, wherein R₅ is (C₄-C₇)alkyl; (C₅-C₁₀)cycloalkyl, —CH₂—(C₅-C₇)cycloalkyl, spiro(C₈-C₁₁)cycloalkyl, or phenyl.
11. The compound according to any one of paragraphs 1-5, or a pharmaceutically acceptable salt thereof, wherein R₅ is
(i) a bridged cycloalkyl substituted with one to four substituents selected from lower alkyl and hydroxyl;
(ii) (C₄-C₆)alkyl substituted with one or two substituents each independently selected from (C₁-C₄)alkyl, fluoro substituted (C₁-C₄)alkyl, methoxy, hydroxy(C₁-C₄)alkyl, methoxy(C₁-C₄)alkyl, ethynyl, cyano, halo, hydroxy and hydroxyl;
(iii) (C₅-C₉)cycloalkyl substituted with one to two substituents each independently selected from (C₁-C₄)alkyl, fluoro-substituted (C₁-C₄)alkyl, methoxy, and hydroxyl;
(iv) —CH₂—(C₅-C₇)cycloalkyl wherein the (C₅-C₇)cycloalkyl is substituted with one to two substituents each independently selected from (C₁-C₄)alkyl, fluoro-substituted (C₁-C₄)alkyl, methoxy and hydroxyl;
(v) spiro(C₈-C₁₁)cycloalkyl substituted with one or two substituents independently selected from lower alkyl and halogen;
(vi) phenyl substituted with one to two substituents each independently selected from (C₁-C₄)alkyl, fluoro substituted (C₁-C₄)alkyl, methoxy, hydroxy(C₁-C₄)alkyl, methoxy(C₁-C₄)alkyl, ethynyl, cyano, halo, or hydroxyl; or
(vii) lower alkyl, substituted with phenyl, said phenyl optionally substituted with one or two substituents each independently selected from lower alkyl, halo-substituted lower alkyl, alkoxy, hydroxy lower alkyl, alkoxy lower alkyl, ethynyl, cyano, halo, or hydroxyl;
(viii) cycloalkyl, cycloalkylene or —CH₂-cycloalkyl, said cycloalkyl, cycloalkylene or —CH₂-cycloalkyl substituted with one or two substituents each independently selected from lower alkyl, halo-substituted lower alkyl, alkoxy, hydroxyl lower alkyl, alkoxy-lower alkyl, ethynyl, cyano, halo, or hydroxyl;
(ix) spiral(C₈-C₁₁)cycloalkyl, substituted with one or two substituents independently selected from lower alkyl and halogen; or
(x) phenyl, substituted with one or two substituents each independently selected from lower alkyl, fluoro-substituted lower alkyl, alkoxy, hydroxyl lower alkyl, alkoxy lower alkyl, ethynyl, cyano, halo, or hydroxyl.
   12. The compound according to any one of paragraphs 1-5, or a pharmaceutically acceptable salt thereof, where R₅ is

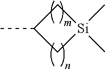

wherein m is 1, 2 or 3 and n is 1, 2, 3, or 4.
13. The compound according to paragraph 12, or a pharmaceutically acceptable salt thereof, wherein R₅ is

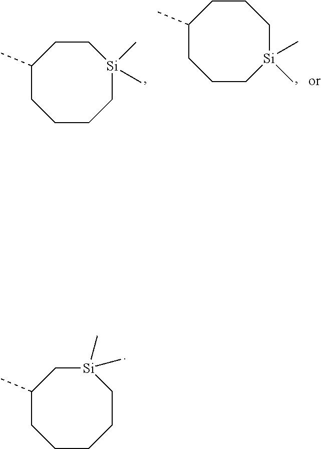

14. The compound according to any one of paragraphs 1-5, or a pharmaceutically acceptable salt thereof, wherein R₅ is a bridged cycloalkyl.
15. The compound according to any one of paragraphs 1-5, or a pharmaceutically acceptable salt thereof, wherein R₅ is a bridged cycloalkyl substituted with one to four substituents selected from lower alkyl and hydroxyl.
16. The compound according to any one of paragraphs 1-5, or a pharmaceutically acceptable salt thereof, wherein R₃NH is

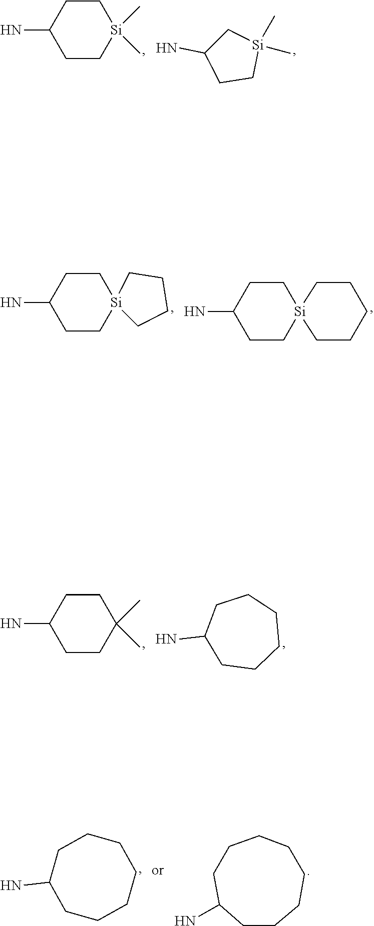

17. The compound according to any one of paragraphs 1-5, or a pharmaceutically acceptable salt thereof, wherein R₃NH is

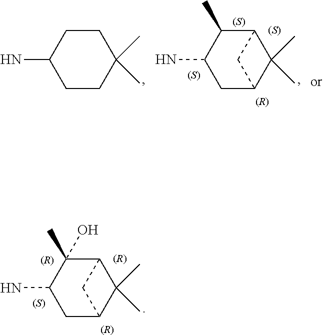

18. The compound according to any one of paragraphs 1-17, or a pharmaceutically acceptable salt thereof, which has Formula (I).
19. The compound according to any one of paragraphs 1-17, or a pharmaceutically acceptable salt thereof, which has Formula (II).
20. The compound of paragraph 1, or a pharmaceutically acceptable salt thereof, which is:
4-(trifluoromethyl)-N-[(1S,2S,3S,5R)-2,6,6-trimethylnorpinan-3-yl]-1H-pyrrolo[2,3-b]pyridine-2-carboxamide;
4-methyl-N-[(1S,2S,3S,5R)-2,6,6-trimethylnorpinan-3-yl]-1H-pyrrolo[2,3-b]pyridine-2-carboxamide;
4-cyclopropyl-N-(4,4-dimethylcyclohexyl)-1H-pyrrolo[2,3-b]pyridine-2-carboxamide;
N-(4,4-dimethylcyclohexyl)-4,6-dimethyl-1H-pyrrolo[2,3-b]pyridine-2-carboxamide;
4-cyclopropyl-N-[(1S,2S,3S,5R)-2,6,6-trimethylnorpinan-3-yl]-1H-pyrrolo[2,3-b]pyridine-2-carboxamide;
4-methyl-N-(4-methylcyclohexyl)-1H-pyrrolo[2,3-b]pyridine-2-carboxamide;
N-cyclooctyl-4-methyl-1H-pyrrolo[2,3-b]pyridine-2-carboxamide;
N-(4,4-dimethylcyclohexyl)-4-(trifluoromethyl)-1H-pyrrolo[2,3-b]pyridine-2-carboxamide;
4-cyano-N-[(1S,2S,3S,5R)-2,6,6-trimethylnorpinan-3-yl]-1H-pyrrolo[2,3-b]pyridine-2-carboxamide;
4,6-dimethyl-N-[(1S,2S,3S,5R)-2,6,6-trimethylnorpinan-3-yl]-1H-pyrrolo[2,3-b]pyridine-2-carboxamide;
4-cyano-N-cyclooctyl-1H-pyrrolo[2,3-b]pyridine-2-carboxamide;
N-cyclooctyl-4-(trifluoromethyl)-1H-pyrrolo[2,3-b]pyridine-2-carboxamide;
N-cyclooctyl-4-cyclopropyl-1H-pyrrolo[2,3-b]pyridine-2-carboxamide;
N-cyclooctyl-4,6-dimethyl-1H-pyrrolo[2,3-b]pyridine-2-carboxamide;
5-chloro-4-fluoro-6-methyl-N-[(1S,2S,3S,5R)-2,6,6-trimethylnorpinan-3-yl]-1H-pyrrolo[2,3-b]pyridine-2-carboxamide;
4-(trifluoromethyl)-N-(1,7,7-trimethylnorbornan-2-yl)-1H-pyrrolo[2,3-b]pyridine-2-carboxamide;
4-chloro-N-(4,4-dimethylcyclohexyl)-6-methyl-1H-pyrrolo[2,3-b]pyridine-2-carboxamide;
5-chloro-N-[(1S,2S,3S,5R)-2,6,6-trimethylnorpinan-3-yl]-1H-pyrrolo[2,3-c]pyridine-2-carboxamide;
5-chloro-N-(4,4-dimethylcyclohexyl)-1H-pyrrolo[2,3-c]pyridine-2-carboxamide;
5,7-dimethyl-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5,7-dimethyl-1H-pyrrolo[2,3-c]pyridine-2-carboxamide;
4-fluoro-5-methyl-N-[(1S,2S,3S,5R)-2,6,6-trimethylnorpinan-3-yl]-1H-pyrrolo[2,3-c]pyridine-2-carboxamide;
N-(4,4-dimethylcyclohex-2-en-1-yl)-4-fluoro-1H-pyrrolo[2,3-b]pyridine-2-carboxamide;
N-(3-bicyclo[3.2.1]octanyl)-4-chloro-1H-pyrrolo[2,3-c]pyridine-2-carboxamide;
N-(1,1-dimethylsilinan-4-yl)-6-fluoro-4-methoxy-1H-pyrrolo[2,3-b]pyridine-2-carboxamide;
N-(1,1-dimethylsilinan-4-yl)-4-fluoro-3,6-dimethyl-1H-pyrrolo[2,3-b]pyridine-2-carboxamide;
N-(1,1-dimethylsilinan-4-yl)-4-fluoro-3-methyl-1H-pyrrolo[2,3-b]pyridine-2-carboxamide;
4-fluoro-N-[(1R,2R,3S,5R)-2-hydroxy-2,6,6-trimethyl-norpinan-3-yl]-1H-pyrrolo[2,3-b]pyridine-2-carboxamide;
N-[(1R,2R,3S,5R)-2-hydroxy-2,6,6-trimethyl-norpinan-3-yl]-4-(trifluoro methyl)-1H-pyrrolo[2,3-b]pyridine-2-carboxamide;
4-chloro-N-[(1R,2R,3S,5R)-2-hydroxy-2,6,6-trimethyl-norpinan-3-yl]-6-methyl-1H-pyrrolo[2,3-b]pyridine-2-carboxamide;
4,5-difluoro-N-[(1R,2R,3S,5R)-2-hydroxy-2,6,6-trimethyl-norpinan-3-yl]-1H-pyrrolo[2,3-b]pyridine-2-carboxamide;
4-fluoro-N-[(1R,2R,3S,5R)-2-hydroxy-2,6,6-trimethyl-norpinan-3-yl]-1H-pyrrolo[2,3-c]pyridine-2-carboxamide;
4-chloro-N-[(1R,2R, 3S, 5R)-2-hydroxy-2,6,6-trimethyl-norpinan-3-yl]-1H-Pyrrolo[2,3-c]pyridine-2-carboxamide;
N-[(1R,2R,3S,5R)-2-hydroxy-2,6,6-trimethyl-norpinan-3-yl]-4-methoxy-1H-pyrrolo[2,3-c]pyridine-2-carboxamide;
N-(1,1-dimethylsilinan-4-yl)-4-fluoro-3-methyl-1H-pyrrolo[2,3-c]pyridine-2-carboxamide;
N-(1, 1-dimethylsilinan-4-yl)-4, 5-difluoro-6-methyl-1H-pyrrolo[2, 3-b]pyridine-2-carboxamide;
4-chloro-N-spiro[3.5]nonan-7-yl-1H-pyrrolo[2,3-b]pyridine-2-carboxamide;
4-fluoro-N-spiro[3.5]nonan-7-yl-1H-pyrrolo[2,3-b]pyridine-2-carboxamide;
4-chloro-N-(4,4-dimethylcyclohexyl)-1H-pyrrolo[2,3-c]pyridine-2-carboxamide;
4-chloro-N-[(1S,2S,3S,5R)-2,6,6-trimethylnorpinan-3-yl]-1H-pyrrolo[2,3-c]Pyridine-2-carboxamide;
4-bromo-N-(4,4-dimethylcyclohexyl)-1H-pyrrolo[2,3-b]pyridine-2-carboxamide;
4-cyano-N-(4,4-dimethylcyclohexyl)-1H-pyrrolo[2,3-b]pyridine-2-carboxamide;
N-(4,4-dimethylcyclohexyl)-4-methoxy-1H-pyrrolo[2,3-b]pyridine-2-carboxamide;
4-bromo-N-[(1S,2S,3S,5R)-2,6,6-trimethylnorpinan-3-yl]-1H-pyrrolo[2,3-b]pyridine-2-carboxamide;
4-methoxy-N-[(1S,2S,3S,5R)-2,6,6-trimethylnorpinan-3-yl]-1H-pyrrolo[2,3-b]pyridine-2-carboxamide;
N-(4,4-dimethylcyclohexyl)-4-(methylamino)-1H-pyrrolo[2,3-b]pyridine-2-carboxamide;
4-(methylamino)-N-[(1S,2S,3S,5R)-2,6,6-trimethylnorpinan-3-yl]-1H-pyrrolo[2,3-b]pyridine-2-carboxamide;
N-cyclooctyl-4-fluoro-1H-pyrrolo[2,3-b]pyridine-2-carboxamide;
4-fluoro-N-(4-methylcyclohexyl)-1H-pyrrolo[2,3-b]pyridine-2-carboxamide;
4-chloro-N-cyclooctyl-1H-pyrrolo[2,3-b]pyridine-2-carboxamide;
4-chloro-N-cyclooctyl-1H-pyrrolo[2,3-c]pyridine-2-carboxamide;
4-bromo-N-cyclooctyl-1H-pyrrolo[2,3-b]pyridine-2-carboxamide;
N-cyclooctyl-4-methoxy-1H-pyrrolo[2,3-b]pyridine-2-carboxamide;
N-cyclooctyl-4-(methylamino)-1H-pyrrolo[2,3-b]pyridine-2-carboxamide;
4-fluoro-6-methyl-N-[(1S,2S,3S,5R)-2,6,6-trimethylnorpinan-3-yl]-1H-pyrrolo[2,3-b]pyridine-2-carboxamide;
N-cyclooctyl-4-fluoro-6-methyl-1H-pyrrolo[2,3-b]pyridine-2-carboxamide;
4-chloro-6-methyl-N-[(1S,2S,3S,5R)-2,6,6-trimethylnorpinan-3-yl]-1H-pyrrolo[2,3-b]pyridine-2-carboxamide;
4,5-difluoro-N-[(1S,2S,3S,5R)-2,6,6-trimethylnorpinan-3-yl]-1H-pyrrolo[2,3-b]pyridine-2-carboxamide;
N-(4,4-dimethylcyclohexyl)-4,5-difluoro-1H-pyrrolo[2,3-b]pyridine-2-carboxamide;
5-chloro-6-fluoro-N-[(1S,2S,3S,5R)-2,6,6-trimethylnorpinan-3-yl]-1H-pyrrolo[2,3-b]pyridine-2-carboxamide;
5-chloro-N-(4,4-dimethylcyclohexyl)-6-fluoro-1H-pyrrolo[2,3-b]pyridine-2-carboxamide;
5-chloro-N-cyclooctyl-6-fluoro-1H-pyrrolo[2,3-b]pyridine-2-carboxamide;
4,5-difluoro-6-methyl-N-[(1S,2S,3S,5R)-2,6,6-trimethylnorpinan-3-yl]-1H-pyrrolo[2,3-b]pyridine-2-carboxamide;
4-chloro-N-[(1S,2S,3S,5R)-2,6,6-trimethylnorpinan-3-yl]-1H-pyrrolo[2,3-c]pyridine-2-carboxamide;
5,6-dimethyl-N-((1S,2S,3S,5R)-2,6,6-trimethylbicyclo[3.1.1]heptan-3-yl)-1H-pyrrolo[2,3-b]pyridine-2-carboxamide;
N-cyclooctyl-5,6-dimethyl-1H-pyrrolo[2,3-b]pyridine-2-carboxamide;
4-fluoro-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-4-fluoro-1H-pyrrolo[2,3-c]pyridine-2-carboxamide;
N-(4,4-dimethylcyclohexyl)-4-fluoro-1H-pyrrolo[2,3-c]pyridine-2-carboxamide;
4-fluoro-N-[(1S,2S,3S,5R)-2,6,6-trimethylnorpinan-3-yl]-1H-pyrrolo[2,3-c]pyridine-2-carboxamide;
4-cyano-N-cyclooctyl-1H-pyrrolo[2,3-c]pyridine-2-carboxamide;
4-cyano-N-(4,4-dimethylcyclohexyl)-1H-pyrrolo[2,3-c]pyridine-2-carboxamide;
4-fluoro-N-(1,7,7-trimethylnorbornan-2-yl)-1H-pyrrolo[2,3-b]pyridine-2-carboxamide;
4-cyano-N-(1,7,7-trimethylnorbornan-2-yl)-1H-pyrrolo[2,3-b]pyridine-2-carboxamide;
N-(1,1-dimethylsilinan-4-yl)-4-fluoro-1H-pyrrolo[2,3-b]pyridine-2-carboxamide;
4-cyano-N-(1,1-dimethylsilinan-4-yl)-1H-pyrrolo[2,3-b]pyridine-2-carboxamide;
N-(1,1-dimethylsilinan-4-yl)-4-(trifluoromethyl)-1H-pyrrolo[2,3-b]pyridine-2-carboxamide;
N-cyclooctyl-4-fluoro-6-methyl-1H-pyrrolo[2,3-b]pyridine-2-carboxamide;
5-methoxy-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5-methoxy-1H-pyrrolo[2,3-c]pyridine-2-carboxamide;
7-fluoro-5-methyl-N-[(1S,2S,3S,5R)-2,6,6-trimethylnorpinan-3-yl]-1H-pyrrolo[2,3-c]pyridine-2-carboxamide;
5-methyl-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5-methyl-1H-pyrrolo[2,3-c]pyridine-2-carboxamide;
5-fluoro-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5-fluoro-1H-pyrrolo[2,3-c]pyridine-2-carboxamide;
2-[[(1S,2S,3S,5R)-2,6,6-trimethylnorpinan-3-yl]carbamoyl]-1H-pyrrolo[2,3-c]pyridine-5-carboxylic acid;
2-[(4,4-dimethylcyclohexyl)carbamoyl]-1H-pyrrolo[2,3-c]pyridine-5-carboxylic acid;
N2-(4,4-dimethylcyclohexyl)-1H-pyrrolo[2,3-c]pyridine-2,5-dicarboxamide;
N2-[(1S,2S,3S,5R)-2,6,6-trimethylnorpinan-3-yl]-1H-pyrrolo[2,3-c]pyridine-2,5-dicarboxamide;
5-fluoro-7-methyl-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5-fluoro-7-methyl-1H-pyrrolo[2,3-c]pyridine-2-carboxamide;
5-chloro-4-fluoro-N-[(1S,2S,3 S,5R)-2,6,6-trimethylnorpinan-3-yl]-1H-pyrrolo[2,3-c]pyridine-2-carboxamide;
5-chloro-N-(4,4-dimethylcyclohexyl)-4-fluoro-1H-pyrrolo[2,3-c]pyridine-2-carboxamide;
N-(4,4-dimethylcyclohexyl)-4-fluoro-5-methyl-1H-pyrrolo[2,3-c]pyridine-2-carboxamide;
4-fluoro-N-spiro[2.5]octan-6-yl-1H-pyrrolo[2,3-b]pyridine-2-carboxamide;
N-(2,2-difluorospiro[2.5]octan-6-yl)-4-fluoro-1H-pyrrolo[2,3-b]pyridine-2-carboxamide;
N-(4,4-dimethylcyclohexyl)-5-methyl-1H-pyrrolo[2,3-c]pyridine-2-carboxamide;
4-fluoro-N-(4-fluoro-4-methyl-cyclohexyl)-1H-pyrrolo[2,3-b]pyridine-2-carboxamide;
N-(4-bicyclo[2.2.2]octanyl)-4-fluoro-1H-pyrrolo[2,3-b]pyridine-2-carboxamide;
4-chloro-N-spiro[2.5]octan-6-yl-1H-pyrrolo[2,3-c]pyridine-2-carboxamide;
4-chloro-N-(1,1-difluorospiro[2.5]octan-6-yl)-1H-pyrrolo[2,3-c]pyridine-2-carboxamide;
4-chloro-N-(4-fluoro-4-methyl-cyclohexyl)-1H-pyrrolo[2,3-c]pyridine-2-carboxamide;
4-chloro-N-(4,4-dimethylcyclohex-2-en-1-yl)-1H-pyrrolo[2,3-c]pyridine-2-carboxamide;
N-(4-bicyclo[2.2.2]octanyl)-4-chloro-1H-pyrrolo[2,3-c]pyri-dine-2-carboxamide;
4-fluoro-3-methyl-N-[(1S,2S,3S,5R)-2,6,6-trimethylnorpinan-3-yl]-1H-pyrrolo[2,3-b]pyridine-2-carboxamide;
4-chloro-6-oxido-N-[(1S,2S,3S,5R)-2,6,6-trimethyl-norpinan-3-yl]-1H-pyrrolo[2,3-c]pyridin-6-ium-2-carboxamide;
4-fluoro-N-(1,7,7-trimethylnorbornan-2-yl)-1H-pyrrolo[2,3-c]pyridine-2-carboxamide;
4-chloro-N-(1,7,7-trimethylnorbornan-2-yl)-1H-pyrrolo[2,3-c]pyridine-2-carboxamide;
N-(1,1-dimethylsilinan-4-yl)-4-fluoro-1H-pyrrolo[2,3-c]pyridine-2-carboxamide;
4-chloro-N-(1,1-dimethylsilinan-4-yl)-1H-pyrrolo[2,3-c]pyridine-2-carboxamide;
4-fluoro-6-methyl-N-(1,7,7-trimethylnorbornan-2-yl)-1H-pyrrolo[2,3-b]pyridine-2-carboxamide;
4-chloro-6-methyl-N-(1,7,7-trimethylnorbornan-2-yl)-1H-pyrrolo[2,3-b]pyridine-2-carboxamide;
N-(1,1-dimethylsilinan-4-yl)-6-fluoro-4-methoxy-1H-indole-2-carboxamide;
N-(1,1-dimethylsilinan-4-yl)-4-methoxy-1H-pyrrolo[2,3-c]pyridine-2-carboxamide;
4-chloro-N-(1,1-dimethylsilinan-4-yl)-6-methyl-1H-pyrrolo[2,3-b]pyridine-2-carboxamide;
4-cyano-N-[(1R,2R,3S,5R)-2-hydroxy-2,6,6-trimethyl-norpinan-3-yl]-1H-pyrrolo[2,3-b]pyridine-2-carboxamide;
5-chloro-N-[(1S,2S,3S,5R)-2,6,6-trimethylnorpinan-3-yl]-1H-pyrrolo[2,3-c]pyridine-2-carboxamide;
5-chloro-N-(4,4-dimethylcyclohexyl)-4-fluoro-6-methyl-1H-pyrrolo[2,3-b]pyridine-2-carboxamide;
N-(1,1-dimethylsilinan-4-yl)-4-fluoro-6-methyl-1H-pyrrolo[2,3-b]pyridine-2-carboxamide;
N-(1,1-dimethylsilinan-4-yl)-5-fluoro-4,6-dimethyl-1H-pyrrolo[2,3-b]pyridine-2-carboxamide;
4,5-dichloro-N-(1,1-dimethylsilinan-4-yl)-7-methyl-1H-pyrrolo[2,3-c]pyridine-2-carboxamide;
5-chloro-N-(1,1-dimethylsilinan-4-yl)-4-fluoro-7-methyl-1H-pyrrolo[2,3-c]pyridine-2-carboxamide;
4,5-dichloro-N-(1,1-dimethylsilinan-4-yl)-6-methyl-1H-pyrrolo[2,3-b]pyridine-2-carboxamide;
5-chloro-N-(1,1-dimethylsilinan-4-yl)-4,6-dimethyl-1H-pyrrolo[2,3-b]pyridine-2-carboxamide;
4-fluoro-6-methyl-N-(5-silaspiro[4.5]decan-8-yl)-1H-pyrrolo[2,3-b]pyridine-2-carboxamide;
4-(trifluoromethyl)-N-[(1S,2S,3S,5R)-2,6,6-trimethylnorpinan-3-yl]-1H-pyrrolo[2,3-c]pyridine-2-carboxamide;
4-fluoro-N-(5-silaspiro[4.5]decan-8-yl)-1H-pyrrolo[2,3-c]pyridine-2-carboxamide;
4-fluoro-N-(6-silaspiro[5.5]undecan-3-yl)-1H-pyrrolo[2,3-c]pyridine-2-carboxamide;
4-(trifluoromethyl)-N-(1,7,7-trimethylnorbornan-2-yl)-1H-pyrrolo[2,3-c]pyridine-2-carboxamide
N-(1, 1-dimethylsilinan-4-yl)-4, 5-difluoro-6-methyl-1H-pyrrolo[2, 3-b]pyridine-2-carboxamide;
4-chloro-N-(6-silaspiro[5.5]undecan-3-yl)-1H-pyrrolo[2,3-c]pyridine-2-carboxamide;
N-(1,1-dimethylsilinan-4-yl)-4-fluoro-3,6-dimethyl-1H-pyrrolo[2,3-b]pyridine-2-carboxamide;
5-chloro-4-fluoro-N-[(1R,2R,3S,5R)-2-hydroxy-2,6,6-trimethyl-norpinan-3-yl]-6-methyl-1H-pyrrolo[2,3-b]pyridine-2-carboxamide;
5-chloro-N-(1,1-dimethylsilinan-4-yl)-4-fluoro-6-methyl-1H-pyrrolo[2,3-b]pyridine-2-carboxamide;
4-chloro-N-(1,1-dimethylsilepan-4-yl)-6-methyl-1H-pyrrolo[2,3-b]pyridine-2-carboxamide;
4-chloro-N-(1,1-dimethylsilocan-4-yl)-6-methyl-1H-pyrrolo[2,3-b]pyridine-2-carboxamide;
4-chloro-6-methyl-N-(5-silaspiro[4.5]decan-8-yl)-1H-pyrrolo[2,3-b]pyridine-2-carboxamide;
4-chloro-6-methyl-N-(6-silaspiro[5.5]undecan-3-yl)-1H-pyrrolo[2,3-b]pyridine-2-carboxamide;
4-fluoro-3,6-dimethyl-N-(5-silaspiro[4.5]decan-8-yl)-1H-pyrrolo[2,3-b]pyridine-2-carboxamide;
4-fluoro-3,6-dimethyl-N-(6-silaspiro[5.5]undecan-3-yl)-1H-pyrrolo[2,3-b]pyridine-2-carboxamide;
N-(1,1-dimethylsilepan-4-yl)-4,5-difluoro-6-methyl-1H-pyrrolo[2,3-b]pyridine-2-carboxamide;
4,5-difluoro-6-methyl-N-(5-silaspiro[4.5]decan-8-yl)-1H-pyrrolo[2,3-b]pyridine-2-carboxamide;
4,5-difluoro-6-methyl-N-(6-silaspiro[5.5]undecan-3-yl)-1H-pyrrolo[2,3-b]pyridine-2-carboxamide;
N-cyclooctyl-4,5-difluoro-6-methyl-1H-pyrrolo[2,3-b]pyridine-2-carboxamide;
5-chloro-N-(1,1-dimethylsilepan-4-yl)-4-fluoro-6-methyl-1H-pyrrolo[2,3-b]pyridine-2-carboxamide;
4,5-difluoro-N-[(1R,2R,3S,5R)-2-hydroxy-2,6,6-trimethyl-norpinan-3-yl]-6-methyl-1H-pyrrolo[2,3-b]pyridine-2-carboxamide;
N-(1,1-dimethylsilolan-3-yl)-4,5-difluoro-6-methyl-1H-pyrrolo[2,3-b]pyridine-2-carboxamide;
N-[(3R)-1,1-dimethylsilolan-3-yl]-4,5-difluoro-6-methyl-1H-pyrrolo[2,3-b]pyridine-2-carboxamide;
N-[(3S)-1,1-dimethylsilolan-3-yl]-4,5-difluoro-6-methyl-1H-pyrrolo[2,3-b]pyridine-2-carboxamide;
N-(1,1-dimethylsilocan-5-yl)-4,5-difluoro-6-methyl-1H-pyrrolo[2,3-b]pyridine-2-carboxamide;
4-chloro-N-(1,1-dimethylsilocan-5-yl)-6-methyl-1H-pyrrolo[2,3-b]pyridine-2-carboxamide;
4-chloro-N-(1,1-dimethylsilocan-5-ylidene)-6-methyl-1H-pyrrolo[2,3-b]pyridine-2-carboxamide;
5-chloro-N-(1,1-dimethylsilepan-4-yl)-4-fluoro-6-methyl-1H-pyrrolo[2,3-b]pyridine-2-carboxamide;
5-chloro-N-[(4R)-1,1-dimethylsilepan-4-yl]-4-fluoro-6-methyl-1H-pyrrolo[2,3-b]pyridine-2-carboxamide;
5-chloro-N-[(4S)-1,1-dimethylsilepan-4-yl]-4-fluoro-6-methyl-1H-pyrrolo[2,3-b]pyridine-2-carboxamide;
N-(1,1-dimethylsilinan-4-yl)-6-methoxy-1H-pyrrolo[2,3-b]pyridine-2-carboxamide;
N-(1,1-dimethylsilinan-4-yl)-6-phenyl-1H-pyrrolo[2,3-b]pyridine-2-carboxamide;
N-(1,1-dimethylsilinan-4-yl)-6-(3-pyridyl)-1H-pyrrolo[2,3-b]pyridine-2-carboxamide;
N-(1, 1-dimethylsilinan-4-yl)-5-methoxy-1H-pyrrolo[2,3-b]pyridine-2-carboxamide;
N-(1,1-dimethylsilinan-4-yl)-5-phenyl-1H-pyrrolo[2,3-b]pyridine-2-carboxamide;
N-(1,1-dimethylsilinan-4-yl)-5-(3-pyridyl)-1H-pyrrolo[2,3-b]pyridine-2-carboxamide;
4-chloro-N-(1,1-dimethylsilolan-3-yl)-6-methyl-1H-pyrrolo[2,3-b]pyridine-2-carboxamide;
4-chloro-N-[(3R)-1,1-dimethylsilolan-3-yl]-6-methyl-1H-pyrrolo[2,3-b]pyridine-2-carboxamide;
4-chloro-N-[(3S)-1,1-dimethylsilolan-3-yl]-6-methyl-1H-pyrrolo[2,3-b]pyridine-2-carboxamide;
5-(2-fluorophenyl)-N-(6-silaspiro[5.5]undecan-3-yl)-1H-pyrrolo[2,3-b]pyridine-2-carboxamide;
5-(3-pyridyl)-N-(6-silaspiro[5.5]undecan-3-yl)-1H-pyrrolo[2,3-b]pyridine-2-carboxamide;
N-(4,4-dimethylcyclohexyl)-6-methoxy-1H-pyrrolo[2,3-b]pyridine-2-carboxamide;
6-methoxy-N-(5-silaspiro[4.5]decan-8-yl)-1H-pyrrolo[2,3-b]pyridine-2-carboxamide;
6-methoxy-N-(6-silaspiro[5.5]undecan-3-yl)-1H-pyrrolo[2,3-b]pyridine-2-carboxamide;
4-chloro-N-(1,1-dimethylsilinan-4-yl)-6-methoxy-1H-pyrrolo[2,3-b]pyridine-2-carboxamide;
N-(1,1-dimethylsilepan-4-yl)-6-methoxy-1H-pyrrolo[2,3-b]pyridine-2-carboxamide;
(S)—N-(1,1-dimethylsilepan-4-yl)-6-methoxy-1H-pyrrolo[2,3-b]pyridine-2-carboxamide;
(R)—N-(1,1-dimethylsilepan-4-yl)-6-methoxy-1H-pyrrolo[2,3-b]pyridine-2-carboxamide;
6-(cyclobutoxy)-N-(1,1-dimethylsilinan-4-yl)-1H-pyrrolo[2,3-b]pyridine-2-carboxamide;
N-(1,1-dimethylsilinan-4-yl)-4-fluoro-6-methoxy-1H-pyrrolo[2,3-b]pyridine-2-carboxamide;
N-(1,1-dimethylsilocan-5-yl)-6-methoxy-1H-pyrrolo[2,3-b]pyridine-2-carboxamide;
N-(1,1-dimethylsilinan-4-yl)-4-fluoro-5,6-dimethyl-1H-pyrrolo[2,3-b]pyridine-2-carboxamide;
N-(1,1-dimethylsilepan-4-yl)-4-fluoro-5-methyl-1H-pyrrolo[2,3-c]pyridine-2-carboxamide;
4-chloro-N-(1,1-dimethylsilinan-4-yl)-5-methyl-1H-pyrrolo[2,3-c]pyridine-2-carboxamide;
4-fluoro-5-methyl-N-(5-silaspiro[4.5]decan-8-yl)-1H-pyrrolo[2,3-c]pyridine-2-carboxamide;
4-fluoro-5-methyl-N-(6-silaspiro[5.5]undecan-3-yl)-1H-pyrrolo[2,3-c]pyridine-2-carboxamide;
N-(1,1-dimethylsilinan-4-yl)-5-(trifluoromethyl)-1H-pyrrolo[2,3-c]pyridine-2-carboxamide;
N-(1,1-dimethylsilinan-4-yl)-4-fluoro-5-(trifluoromethyl)-1H-pyrrolo[2,3-c]pyridine-2-carboxamide;
4-chloro-N-(1,1-dimethylsilinan-4-yl)-5-(trifluoromethyl)-1H-pyrrolo[2,3-c]pyridine-2-carboxamide;
5-chloro-N-(1,1-dimethylsilinan-4-yl)-4-fluoro-1H-pyrrolo[2,3-c]pyridine-2-carboxamide;
5-chloro-N-(1,1-dimethylsilepan-4-yl)-4-fluoro-1H-pyrrolo[2,3-c]pyridine-2-carboxamide;
5-chloro-4-fluoro-N-(6-silaspiro[5.5]undecan-3-yl)-1H-pyrrolo[2,3-c]pyridine-2-carboxamide;
4,5-dichloro-N-(1,1-dimethylsilinan-4-yl)-1H-pyrrolo[2,3-c]pyridine-2-carboxamide;
4,5-dichloro-N-(1,1-dimethylsilepan-4-yl)-1H-pyrrolo[2,3-c]pyridine-2-carboxamide;
N-(1,1-dimethylsilinan-4-yl)-4-fluoro-5-methyl-1H-pyrrolo[2,3-c]pyridine-2-carboxamide;
N-(1,1-dimethylsilepan-4-yl)-4-fluoro-5-(trifluoromethyl)-1H-pyrrolo[2,3-c]pyridine-2-carboxamide;
4-fluoro-N-(5-silaspiro[4.5]decan-8-yl)-5-(trifluoromethyl)-1H-pyrrolo[2,3-c]pyridine-2-carboxamide;
4-fluoro-N-(6-silaspiro[5.5]undecan-3-yl)-5-(trifluoromethyl)-1H-pyrrolo[2,3-c]pyridine-2-carboxamide;
N-(1,1-dimethylsilinan-4-yl)-4-methyl-5-(trifluoromethyl)-1H-pyrrolo[2,3-c]pyridine-2-carboxamide;
N-(1,1-dimethylsilinan-4-yl)-4-methoxy-5-(trifluoromethyl)-1H-pyrrolo[2,3-c]pyridine-2-carboxamide;
N-(1,1-dimethylsilinan-4-yl)-4-Isopropoxy-1H-pyrrolo[2,3-c]pyridine-2-carboxamide;
N-(1,1-dimethylsilolan-3-yl)-5-(trifluoromethyl)-1H-pyrrolo[2,3-c]pyridine-2-carboxamide;
N-(1,1-dimethylsilinan-4-yl)-5-phenyl-1H-pyrrolo[2,3-c]pyridine-2-carboxamide;
N-(1, 1-dimethylsilepan-4-yl)-4-methyl-5-(trifluoromethyl)-1H-pyrrolo[2,3-c]pyridine-2-carboxamide;
N-(1,1-dimethylsilinan-4-yl)-5-methoxy-4-(trifluoromethyl)-1H-pyrrolo[2,3-c]pyridine-2-carboxamide;
N-(1,1-dimethylsilinan-4-yl)-4-fluoro-3,5-dimethyl-1H-pyrrolo[2,3-c]pyridine-2-carboxamide;
N-(1,1-dimethylsilepan-4-yl)-4-fluoro-3,5-dimethyl-1H-pyrrolo[2,3-c]pyridine-2-carboxamide;
or
6-(cyclopropoxy)-N-(1,1-dimethylsilinan-4-yl)-1H-pyrrolo[2,3-b]pyridine-2-carboxamide.
   21. A pharmaceutical composition, comprising a compound of any one of paragraphs 1-20, or a pharmaceutically acceptable salt thereof, and one or more pharmaceutically acceptable carriers and/or additives.
   22. The pharmaceutical composition according to paragraph 21, further comprising one or more additional anti-infective agents.
   23. The pharmaceutical composition according to paragraph 22, wherein said additional anti-infective agent is rifampicin, rifabutin, rifapentene, isoniazid, ethambutol, kanamycin, amikacin, capreomycin, clofazimine, cycloserine, para-aminosalicylic acid, linezolid, sutezolid, bedaquiline, delamanid, pretomanid, moxifloxacin or levofloxacin, or combinations thereof.
   24. A method of treating a mycobacterial infection, comprising the step of administering a therapeutically effective amount of a compound of any one of paragraphs 1-20, or a pharmaceutically acceptable salt thereof, to a patient in need thereof.
   25. The method of paragraph 24, wherein the mycobacterial infection is caused by Mycobacterium tuberculosis, Mycobacterium avium, Mycobacterium kansasii, Mycobacterium abscessus or Mycobacterium chelonae.
   26. The method of paragraph 24, wherein the mycobacterial infection is caused by Mycobacterium tuberculosis.

It is to be understood that the invention is not limited to the particular embodiments of the invention described above, as variations of the particular embodiments may be made and still fall within the scope of the appended claims.

What is claimed is:

1. A compound of Formula (I) or Formula (II): 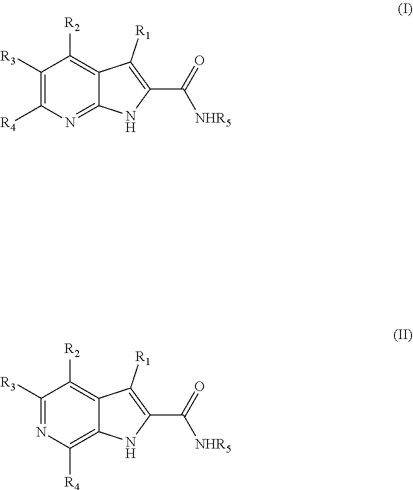
wherein:
R₁ is hydrogen or lower alkyl;
R₂ is hydrogen, lower alkyl, halo, cyano, trifluoromethyl, halo-lower alkyl, di-halo-lower alkyl, alkoxy, or carboxamide;
R₃ is hydrogen, lower alkyl, aryl, heteroaryl, halo, cyano, trifluoromethyl, halo-lower alkyl, di-halo-lower alkyl, alkoxy, or carboxamide;
R₄ is hydrogen, lower alkyl, aryl, heteroaryl, halo, cyano, trifluoromethyl, halo-lower alkyl, di-halo-lower alkyl, alkoxy, cycloalkoxy, or carboxamide;
R₅ is:
(i) lower alkyl;
(ii) cycloalkyl;
(iii) spiral(C₈-C₁₁)cycloalkyl;
(iv) phenyl; 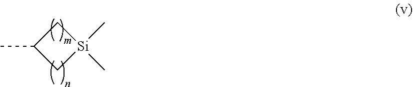
wherein m is 1, 2 or 3 and n is 1, 2, 3, or 4; 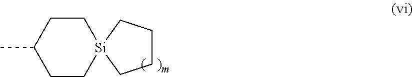
wherein m is 1 or 2;
or
(vii) a bridged cycloalkyl,
or a pharmaceutically acceptable salt thereof.

2. The compound according to claim 1, or a pharmaceutically acceptable salt thereof, wherein R₁ is hydrogen or methyl.

3. The compound according to claim 1, or a pharmaceutically acceptable salt thereof, wherein R₂ is hydrogen, methyl, halo, cyano, trifluoromethyl, mono-fluoromethyl, di-fluoromethyl, methoxy, or carboxamide.

4. The compound according to claim 1, or a pharmaceutically acceptable salt thereof, wherein R₃ is hydrogen, methyl, halo, cyano, trifluoromethyl, mono-fluoromethyl, di-fluoromethyl, methoxy, or carboxamide.

5. The compound according to claim 1, or a pharmaceutically acceptable salt thereof, wherein R₄ is hydrogen, methyl, halo, cyano, trifluoromethyl, mono-fluoromethyl, di-fluoromethyl, methoxy, or carboxamide.

6. The compound according to claim 1, or a pharmaceutically acceptable salt thereof, wherein R₅ is lower alkyl, optionally substituted with phenyl, said phenyl optionally substituted with one or two substituents each independently selected from lower alkyl, halo-substituted lower alkyl, alkoxy, hydroxy lower alkyl, alkoxy lower alkyl, ethynyl, cyano, halo, or hydroxyl.

7. The compound according to claim 1, or a pharmaceutically acceptable salt thereof, wherein R₅ is cycloalkyl, cycloalkylene or —CH₂-cycloalkyl, said cycloalkyl, cycloalkylene or —CH₂-cycloalkyl optionally substituted with one or two substituents each independently selected from lower alkyl, halo-substituted lower alkyl, alkoxy, hydroxyl lower alkyl, alkoxy lower alkyl, ethynyl, cyano, halo, or hydroxyl.

8. The compound according to claim 1, or a pharmaceutically acceptable salt thereof, wherein R₅ is spiral(C₈-C₁₁)cycloalkyl, optionally substituted with one or two substituents selected from lower alkyl and halogen.

9. The compound according to claim 1, or a pharmaceutically acceptable salt thereof, wherein R₅ is

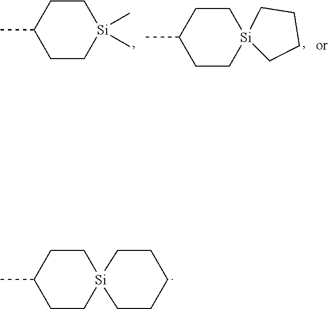

10. The compound according to claim 1, or a pharmaceutically acceptable salt thereof, wherein R₅ is (C₄-C₇)alkyl; (C₅-C₁₀)cycloalkyl, —CH₂—(C₅-C₇)cycloalkyl, spiro(C₈-C₁₁)cycloalkyl, or phenyl.

11. The compound according to claim 1, or a pharmaceutically acceptable salt thereof, wherein R₅ is
(i) a bridged cycloalkyl substituted with one to four substituents selected from lower alkyl and hydroxyl;
(ii) (C₄-C₆)alkyl substituted with one or two substituents each independently selected from (C₁-C₄)alkyl, fluoro substituted (C₁-C₄)alkyl, methoxy, hydroxy(C₁-C₄)alkyl, methoxy(C₁-C₄)alkyl, ethynyl, cyano, halo, hydroxy and hydroxyl;
(iii) (C₅-C₉)cycloalkyl substituted with one to two substituents each independently selected from (C₁-C₄)alkyl, fluoro-substituted (C₁-C₄)alkyl, methoxy, and hydroxyl;
(iv) —CH₂—(C₅-C₇)cycloalkyl wherein the (C₅-C₇)cycloalkyl is substituted with one to two substituents each independently selected from (C₁-C₄)alkyl, fluoro-substituted (C₁-C₄)alkyl, methoxy and hydroxyl;
(v) spiro(C₈-C₁₁)cycloalkyl substituted with one or two substituents independently selected from lower alkyl and halogen;
(vi) phenyl substituted with one to two substituents each independently selected from (C₁-C₄)alkyl, fluoro substituted (C₁-C₄)alkyl, methoxy, hydroxy(C₁-C₄)alkyl, methoxy(C₁-C₄)alkyl, ethynyl, cyano, halo, or hydroxyl;
(vii) lower alkyl, substituted with phenyl, said phenyl optionally substituted with one or two substituents each independently selected from lower alkyl, halo-substituted lower alkyl, alkoxy, hydroxy lower alkyl, alkoxy lower alkyl, ethynyl, cyano, halo, or hydroxyl;
(viii) cycloalkyl, cycloalkylene or —CH₂-cycloalkyl, said cycloalkyl, cycloalkylene or —CH₂-cycloalkyl substituted with one or two substituents each independently selected from lower alkyl, halo-substituted lower alkyl, alkoxy, hydroxyl lower alkyl, alkoxy-lower alkyl, ethynyl, cyano, halo, or hydroxyl; or
(ix) phenyl, substituted with one or two substituents each independently selected from lower alkyl, fluoro-substituted lower alkyl, alkoxy, hydroxyl lower alkyl, alkoxy lower alkyl, ethynyl, cyano, halo, or hydroxyl.

12. The compound according to claim 1, or a pharmaceutically acceptable salt thereof, where R₅ is 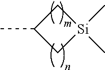
wherein m is 1, 2 or 3 and n is 1, 2, 3, or 4.

13. The compound according to paragraph 12, or a pharmaceutically acceptable salt thereof,
wherein R₅ is

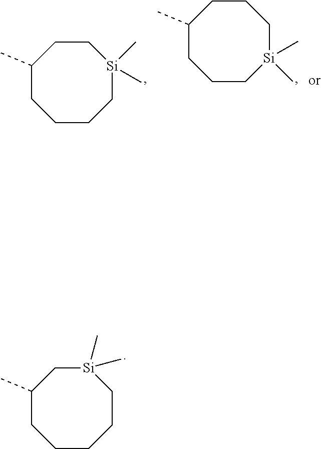

14. The compound according to claim 1, or a pharmaceutically acceptable salt thereof, wherein R₅ is a bridged cycloalkyl.

15. The compound according to claim 1, or a pharmaceutically acceptable salt thereof, wherein R₅ is a bridged cycloalkyl substituted with one to four substituents selected from lower alkyl and hydroxyl.

16. The compound according to claim 1, or a pharmaceutically acceptable salt thereof, wherein R₃NH is

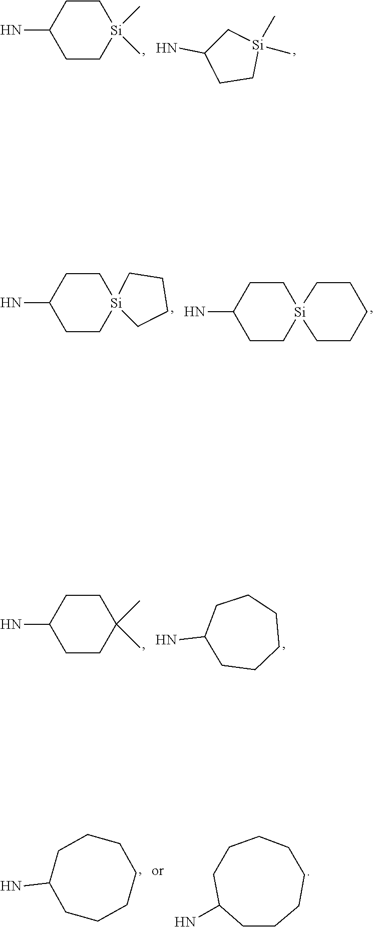

17. The compound according to claim 1, or a pharmaceutically acceptable salt thereof, wherein R₃NH is

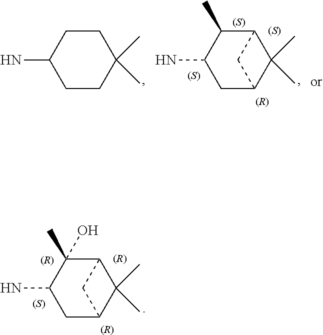

18. The compound according to claim 1, or a pharmaceutically acceptable salt thereof, which has Formula (I).

19. The compound according to claim 1, or a pharmaceutically acceptable salt thereof, which has Formula (II).

20. The compound of claim 1, or a pharmaceutically acceptable salt thereof, which is:
4-(trifluoromethyl)-N-[(1S,2S,3S,5R)-2,6,6-trimethylnorpinan-3-yl]-1H-pyrrolo[2,3-b]pyridine-2-carboxamide;
4-methyl-N-[(1S,2S,3S,5R)-2,6,6-trimethylnorpinan-3-yl]-1H-pyrrolo[2,3-b]pyridine-2-carboxamide;
4-cyclopropyl-N-(4,4-dimethylcyclohexyl)-1H-pyrrolo[2,3-b]pyridine-2-carboxamide;
N-(4,4-dimethylcyclohexyl)-4,6-dimethyl-1H-pyrrolo[2,3-b]pyridine-2-carboxamide;
4-cyclopropyl-N-[(1S,2S,3S,5R)-2,6,6-trimethylnorpinan-3-yl]-1H-pyrrolo[2,3-b]pyridine-2-carboxamide;
4-methyl-N-(4-methylcyclohexyl)-1H-pyrrolo[2,3-b]pyridine-2-carboxamide;
N-cyclooctyl-4-methyl-1H-pyrrolo[2,3-b]pyridine-2-carboxamide;
N-(4,4-dimethylcyclohexyl)-4-(trifluoromethyl)-1H-pyrrolo[2,3-b]pyridine-2-carboxamide;
4-cyano-N-[(1S,2S,3S,5R)-2,6,6-trimethylnorpinan-3-yl]-1H-pyrrolo[2,3-b]pyridine-2-carboxamide;
4,6-dimethyl-N-[(1S,2S,3S,5R)-2,6,6-trimethylnorpinan-3-yl]-1H-pyrrolo[2,3-b]pyridine-2-carboxamide;
4-cyano-N-cyclooctyl-1H-pyrrolo[2,3-b]pyridine-2-carboxamide;
N-cyclooctyl-4-(trifluoromethyl)-1H-pyrrolo[2,3-b]pyridine-2-carboxamide;
N-cyclooctyl-4-cyclopropyl-1H-pyrrolo[2,3-b]pyridine-2-carboxamide;
N-cyclooctyl-4,6-dimethyl-1H-pyrrolo[2,3-b]pyridine-2-carboxamide;
5-chloro-4-fluoro-6-methyl-N-[(1S,2S,3S,5R)-2,6,6-trimethylnorpinan-3-yl]-1H-pyrrolo[2,3-b]pyridine-2-carboxamide;
4-(trifluoromethyl)-N-(1,7,7-trimethylnorbornan-2-yl)-1H-pyrrolo[2,3-b]pyridine-2-carboxamide;
4-chloro-N-(4,4-dimethylcyclohexyl)-6-methyl-1H-pyrrolo[2,3-b]pyridine-2-carboxamide;
5-chloro-N-[(1S,2S,3S,5R)-2,6,6-trimethylnorpinan-3-yl]-1H-pyrrolo[2,3-c]pyridine-2-carboxamide;
5-chloro-N-(4,4-dimethylcyclohexyl)-1H-pyrrolo[2,3-c]pyridine-2-carboxamide;
5,7-dimethyl-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5,7-dimethyl-1H-pyrrolo[2,3-c]pyridine-2-carboxamide;
4-fluoro-5-methyl-N-[(1S,2S,3S,5R)-2,6,6-trimethylnorpinan-3-yl]-1H-pyrrolo[2,3-c]pyridine-2-carboxamide;
N-(4,4-dimethylcyclohex-2-en-1-yl)-4-fluoro-1H-pyrrolo[2,3-b]pyridine-2-carboxamide;
N-(3-bicyclo[3.2.1]octanyl)-4-chloro-1H-pyrrolo[2,3-c]pyridine-2-carboxamide;
N-(1,1-dimethylsilinan-4-yl)-6-fluoro-4-methoxy-1H-pyrrolo[2,3-b]pyridine-2-carboxamide;
N-(1,1-dimethylsilinan-4-yl)-4-fluoro-3, 6-dimethyl-1H-pyrrolo[2,3-b]pyridine-2-carboxamide;
N-(1,1-dimethylsilinan-4-yl)-4-fluoro-3-methyl-1H-pyrrolo[2,3-b]pyridine-2-carboxamide;
4-fluoro-N-[(1R,2R,3 S,5R)-2-hydroxy-2,6,6-trimethyl-norpinan-3-yl]-1H-pyrrolo[2,3-b]pyridine-2-carboxamide;
N-[(1R,2R,3S,5R)-2-hydroxy-2,6,6-trimethyl-norpinan-3-yl]-4-(trifluoro methyl)-1H-pyrrolo[2,3-b]pyridine-2-carboxamide;
4-chloro-N-[(1R,2R,3S,5R)-2-hydroxy-2,6,6-trimethyl-norpinan-3-yl]-6-methyl-1H-pyrrolo[2,3-b]pyridine-2-carboxamide;
4,5-difluoro-N-[(1R,2R,3S,5R)-2-hydroxy-2,6,6-trimethyl-norpinan-3-yl]-1H-pyrrolo[2,3-b]pyridine-2-carboxamide;
4-fluoro-N-[(1R,2R,3S,5R)-2-hydroxy-2,6,6-trimethyl-norpinan-3-yl]-1H-pyrrolo[2,3-c]pyridine-2-carboxamide;
4-chloro-N-[(1R,2R, 3S, 5R)-2-hydroxy-2,6,6-trimethyl-norpinan-3-yl]-1H-Pyrrolo[2,3-c]pyridine-2-carboxamide;
N-[(1R,2R,3 S,5R)-2-hydroxy-2,6,6-trimethyl-norpinan-3-yl]-4-methoxy-1H-pyrrolo[2,3-c]pyridine-2-carboxamide;
N-(1,1-dimethylsilinan-4-yl)-4-fluoro-3-methyl-1H-pyrrolo[2,3-c]pyridine-2-carboxamide;
N-(1, 1-dimethylsilinan-4-yl)-4, 5-difluoro-6-methyl-1H-pyrrolo[2, 3-b]pyridine-2-carboxamide;
4-chloro-N-spiro[3.5]nonan-7-yl-1H-pyrrolo[2,3-b]pyridine-2-carboxamide;
4-fluoro-N-spiro[3.5]nonan-7-yl-1H-pyrrolo[2,3-b]pyridine-2-carboxamide;
4-chloro-N-(4,4-dimethylcyclohexyl)-1H-pyrrolo[2,3-c]pyridine-2-carboxamide;
4-chloro-N-[(1S,2S,3S,5R)-2,6,6-trimethylnorpinan-3-yl]-1H-pyrrolo[2,3-c]Pyridine-2-carboxamide;
4-bromo-N-(4,4-dimethylcyclohexyl)-1H-pyrrolo[2,3-b]pyridine-2-carboxamide;
4-cyano-N-(4,4-dimethylcyclohexyl)-1H-pyrrolo[2,3-b]pyridine-2-carboxamide;
N-(4,4-dimethylcyclohexyl)-4-methoxy-1H-pyrrolo[2,3-b]pyridine-2-carboxamide;
4-bromo-N-[(1S,2S,3S,5R)-2,6,6-trimethylnorpinan-3-yl]-1H-pyrrolo[2,3-b]pyridine-2-carboxamide;
4-methoxy-N-[(1S,2S,3S,5R)-2,6,6-trimethylnorpinan-3-yl]-1H-pyrrolo[2,3-b]pyridine-2-carboxamide;
N-(4,4-dimethylcyclohexyl)-4-(methylamino)-1H-pyrrolo[2,3-b]pyridine-2-carboxamide;
4-(methylamino)-N-[(1S,2S,3S,5R)-2,6,6-trimethylnorpinan-3-yl]-1H-pyrrolo[2,3-b]pyridine-2-carboxamide;
N-cyclooctyl-4-fluoro-1H-pyrrolo[2,3-b]pyridine-2-carboxamide;
4-fluoro-N-(4-methylcyclohexyl)-1H-pyrrolo[2,3-b]pyridine-2-carboxamide;
4-chloro-N-cyclooctyl-1H-pyrrolo[2,3-b]pyridine-2-carboxamide;
4-chloro-N-cyclooctyl-1H-pyrrolo[2,3-c]pyridine-2-carboxamide;
4-bromo-N-cyclooctyl-1H-pyrrolo[2,3-b]pyridine-2-carboxamide;
N-cyclooctyl-4-methoxy-1H-pyrrolo[2,3-b]pyridine-2-carboxamide;
N-cyclooctyl-4-(methylamino)-1H-pyrrolo[2,3-b]pyridine-2-carboxamide;
4-fluoro-6-methyl-N-[(1S,2S,3S,5R)-2,6,6-trimethylnorpinan-3-yl]-1H-pyrrolo[2,3-b]pyridine-2-carboxamide;
N-cyclooctyl-4-fluoro-6-methyl-1H-pyrrolo[2,3-b]pyridine-2-carboxamide;
4-chloro-6-methyl-N-[(1S,2S,3S,5R)-2,6,6-trimethylnorpinan-3-yl]-1H-pyrrolo[2,3-b]pyridine-2-carboxamide;
4,5-difluoro-N-[(1S,2S,3S,5R)-2,6,6-trimethylnorpinan-3-yl]-1H-pyrrolo[2,3-b]pyridine-2-carboxamide;
N-(4,4-dimethylcyclohexyl)-4,5-difluoro-1H-pyrrolo[2,3-b]pyridine-2-carboxamide;
5-chloro-6-fluoro-N-[(1S,2S,3S,5R)-2,6,6-trimethylnorpinan-3-yl]-1H-pyrrolo[2,3-b]pyridine-2-carboxamide;
5-chloro-N-(4,4-dimethylcyclohexyl)-6-fluoro-1H-pyrrolo[2,3-b]pyridine-2-carboxamide;
5-chloro-N-cyclooctyl-6-fluoro-1H-pyrrolo[2,3-b]pyridine-2-carboxamide;
4,5-difluoro-6-methyl-N-[(1S,2S,3S,5R)-2,6,6-trimethylnorpinan-3-yl]-1H-pyrrolo[2,3-b]pyridine-2-carboxamide;
4-chloro-N-[(1S,2S,3S,5R)-2,6,6-trimethylnorpinan-3-yl]-1H-pyrrolo[2,3-c]pyridine-2-carboxamide;
5,6-dimethyl-N-((1S,2S,3S,5R)-2,6,6-trimethylbicyclo[3.1.1]heptan-3-yl)-1H-pyrrolo[2,3-b]pyridine-2-carboxamide;
N-cyclooctyl-5, 6-dimethyl-1H-pyrrolo[2,3-b]pyridine-2-carboxamide;
4-fluoro-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-4-fluoro-1H-pyrrolo[2,3-c]pyridine-2-carboxamide;
N-(4,4-dimethylcyclohexyl)-4-fluoro-1H-pyrrolo[2,3-c]pyridine-2-carboxamide;
4-fluoro-N-[(1S,2S,3S,5R)-2,6,6-trimethylnorpinan-3-yl]-1H-pyrrolo[2,3-c]pyridine-2-carboxamide;
4-cyano-N-cyclooctyl-1H-pyrrolo[2,3-c]pyridine-2-carboxamide;
4-cyano-N-(4,4-dimethylcyclohexyl)-1H-pyrrolo[2,3-c]pyridine-2-carboxamide;
4-fluoro-N-(1,7,7-trimethylnorbornan-2-yl)-1H-pyrrolo[2,3-b]pyridine-2-carboxamide;
4-cyano-N-(1,7,7-trimethylnorbornan-2-yl)-1H-pyrrolo[2,3-b]pyridine-2-carboxamide;
N-(1,1-dimethylsilinan-4-yl)-4-fluoro-1H-pyrrolo[2,3-b]pyridine-2-carboxamide;
4-cyano-N-(1,1-dimethylsilinan-4-yl)-1H-pyrrolo[2,3-b]pyridine-2-carboxamide;
N-(1,1-dimethylsilinan-4-yl)-4-(trifluoromethyl)-1H-pyrrolo[2,3-b]pyridine-2-carboxamide;
N-cyclooctyl-4-fluoro-6-methyl-1H-pyrrolo[2,3-b]pyridine-2-carboxamide;
5-methoxy-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5-methoxy-1H-pyrrolo[2,3-c]pyridine-2-carboxamide;
7-fluoro-5-methyl-N-[(1S,2S,3S,5R)-2,6,6-trimethylnorpinan-3-yl]-1H-pyrrolo[2,3-c]pyridine-2-carboxamide;
5-methyl-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5-methyl-1H-pyrrolo[2,3-c]pyridine-2-carboxamide;
5-fluoro-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5-fluoro-1H-pyrrolo[2,3-c]pyridine-2-carboxamide;
2-[[(1S,2S,3S,5R)-2,6,6-trimethylnorpinan-3-yl]carbamoyl]-1H-pyrrolo[2,3-c]pyridine-5-carboxylic acid;
2-[(4,4-dimethylcyclohexyl)carbamoyl]-1H-pyrrolo[2,3-c]pyridine-5-carboxylic acid;
N2-(4,4-dimethylcyclohexyl)-1H-pyrrolo[2,3-c]pyridine-2,5-dicarboxamide;
N2-[(1S,2S,3S,5R)-2,6,6-trimethylnorpinan-3-yl]-1H-pyrrolo[2,3-c]pyridine-2,5-dicarboxamide;
5-fluoro-7-methyl-N-[(1S,2S,3S,5R)-2,6,6-trimethylnorpinan-3-yl]-1H-pyrrolo[2,3-c]pyridine-2-carboxamide;
N-(4,4-dimethylcyclohexyl)-5-fluoro-7-methyl-1H-pyrrolo[2,3-c]pyridine-2-carboxamide;
5-chloro-4-fluoro-N-[(1S,2S,3S,5R)-2,6,6-trimethylnorpinan-3-yl]-1H-pyrrolo[2,3-c]pyridine-2-carboxamide;
5-chloro-N-(4,4-dimethylcyclohexyl)-4-fluoro-1H-pyrrolo[2,3-c]pyridine-2-carboxamide;
N-(4,4-dimethylcyclohexyl)-4-fluoro-5-methyl-1H-pyrrolo[2,3-c]pyridine-2-carboxamide;
4-fluoro-N-spiro[2.5]octan-6-yl-1H-pyrrolo[2,3-b]pyridine-2-carboxamide;
N-(2,2-difluorospiro[2.5]octan-6-yl)-4-fluoro-1H-pyrrolo[2,3-b]pyridine-2-carboxamide;
N-(4,4-dimethylcyclohexyl)-5-methyl-1H-pyrrolo[2,3-c]pyridine-2-carboxamide;
4-fluoro-N-(4-fluoro-4-methyl-cyclohexyl)-1H-pyrrolo[2,3-b]pyridine-2-carboxamide;
N-(4-bicyclo[2.2.2]octanyl)-4-fluoro-1H-pyrrolo[2,3-b]pyridine-2-carboxamide;
4-chloro-N-spiro[2.5]octan-6-yl-1H-pyrrolo[2,3-c]pyridine-2-carboxamide;
4-chloro-N-(1,1-difluorospiro[2.5]octan-6-yl)-1H-pyrrolo[2,3-c]pyridine-2-carboxamide;
4-chloro-N-(4-fluoro-4-methyl-cyclohexyl)-1H-pyrrolo[2,3-c]pyridine-2-carboxamide;
4-chloro-N-(4,4-dimethylcyclohex-2-en-1-yl)-1H-pyrrolo[2,3-c]pyridine-2-carboxamide;
N-(4-bicyclo[2.2.2]octanyl)-4-chloro-1H-pyrrolo[2,3-c]pyri-dine-2-carboxamide;
4-fluoro-3-methyl-N-[(1S,2S,3S,5R)-2,6,6-trimethylnorpinan-3-yl]-1H-pyrrolo[2,3-b]pyridine-2-carboxamide;
4-chloro-6-oxido-N-[(1S,2S,3S,5R)-2,6,6-trimethyl-norpinan-3-yl]-1H-pyrrolo[2,3-c]pyridin-6-ium-2-carboxamide;
4-fluoro-N-(1,7,7-trimethylnorbornan-2-yl)-1H-pyrrolo[2,3-c]pyridine-2-carboxamide;
4-chloro-N-(1,7,7-trimethylnorbornan-2-yl)-1H-pyrrolo[2,3-c]pyridine-2-carboxamide;
N-(1,1-dimethylsilinan-4-yl)-4-fluoro-1H-pyrrolo[2,3-c]pyridine-2-carboxamide;
4-chloro-N-(1,1-dimethylsilinan-4-yl)-1H-pyrrolo[2,3-c]pyridine-2-carboxamide;
4-fluoro-6-methyl-N-(1,7,7-trimethylnorbornan-2-yl)-1H-pyrrolo[2,3-b]pyridine-2-carboxamide;
4-chloro-6-methyl-N-(1,7,7-trimethylnorbornan-2-yl)-1H-pyrrolo[2,3-b]pyridine-2-carboxamide;
N-(1,1-dimethylsilinan-4-yl)-6-fluoro-4-methoxy-1H-indole-2-carboxamide;
N-(1,1-dimethylsilinan-4-yl)-4-methoxy-1H-pyrrolo[2,3-c]pyridine-2-carboxamide;
4-chloro-N-(1,1-dimethylsilinan-4-yl)-6-methyl-1H-pyrrolo[2,3-b]pyridine-2-carboxamide;
4-cyano-N-[(1R,2R,3S,5R)-2-hydroxy-2,6,6-trimethyl-norpinan-3-yl]-1H-pyrrolo[2,3-b]pyridine-2-carboxamide;
5-chloro-N-[(1S,2S,3S,5R)-2,6,6-trimethylnorpinan-3-yl]-1H-pyrrolo[2,3-c]pyridine-2-carboxamide;
5-chloro-N-(4,4-dimethylcyclohexyl)-4-fluoro-6-methyl-1H-pyrrolo[2,3-b]pyridine-2-carboxamide;
N-(1,1-dimethylsilinan-4-yl)-4-fluoro-6-methyl-1H-pyrrolo[2,3-b]pyridine-2-carboxamide;
N-(1,1-dimethylsilinan-4-yl)-5-fluoro-4,6-dimethyl-1H-pyrrolo[2,3-b]pyridine-2-carboxamide;
4,5-dichloro-N-(1,1-dimethylsilinan-4-yl)-7-methyl-1H-pyrrolo[2,3-c]pyridine-2-carboxamide;
5-chloro-N-(1,1-dimethylsilinan-4-yl)-4-fluoro-7-methyl-1H-pyrrolo[2,3-c]pyridine-2-carboxamide;
4,5-dichloro-N-(1,1-dimethylsilinan-4-yl)-6-methyl-1H-pyrrolo[2,3-b]pyridine-2-carboxamide;
5-chloro-N-(1,1-dimethylsilinan-4-yl)-4,6-dimethyl-1H-pyrrolo[2,3-b]pyridine-2-carboxamide;
4-fluoro-6-methyl-N-(5-silaspiro[4.5]decan-8-yl)-1H-pyrrolo[2,3-b]pyridine-2-carboxamide;
4-(trifluoromethyl)-N-[(1S,2S,3S,5R)-2,6,6-trimethylnorpinan-3-yl]-1H-pyrrolo[2,3-c]pyridine-2-carboxamide;
4-fluoro-N-(5-silaspiro[4.5]decan-8-yl)-1H-pyrrolo[2,3-c]pyridine-2-carboxamide;
4-fluoro-N-(6-silaspiro[5.5]undecan-3-yl)-1H-pyrrolo[2,3-c]pyridine-2-carboxamide;
4-(trifluoromethyl)-N-(1,7,7-trimethylnorbornan-2-yl)-1H-pyrrolo[2,3-c]pyridine-2-carboxamide N-(1, 1-dimethylsilinan-4-yl)-4, 5-difluoro-6-methyl-1H-pyrrolo[2, 3-b]pyridine-2-carboxamide;
4-chloro-N-(6-silaspiro[5.5]undecan-3-yl)-1H-pyrrolo[2,3-c]pyridine-2-carboxamide;
N-(1,1-dimethylsilinan-4-yl)-4-fluoro-3, 6-dimethyl-1H-pyrrolo[2,3-b]pyridine-2-carboxamide;
5-chloro-4-fluoro-N-[(1R,2R,3S,5R)-2-hydroxy-2,6,6-trimethyl-norpinan-3-yl]-6-methyl-1H-pyrrolo[2,3-b]pyridine-2-carboxamide;
5-chloro-N-(1,1-dimethylsilinan-4-yl)-4-fluoro-6-methyl-1H-pyrrolo[2,3-b]pyridine-2-carboxamide;
4-chloro-N-(1,1-dimethylsilepan-4-yl)-6-methyl-1H-pyrrolo[2,3-b]pyridine-2-carboxamide;
4-chloro-N-(1,1-dimethylsilocan-4-yl)-6-methyl-1H-pyrrolo[2,3-b]pyridine-2-carboxamide;
4-chloro-6-methyl-N-(5-silaspiro[4.5]decan-8-yl)-1H-pyrrolo[2,3-b]pyridine-2-carboxamide;
4-chloro-6-methyl-N-(6-silaspiro[5.5]undecan-3-yl)-1H-pyrrolo[2,3-b]pyridine-2-carboxamide;
4-fluoro-3,6-dimethyl-N-(5-silaspiro[4.5]decan-8-yl)-1H-pyrrolo[2,3-b]pyridine-2-carboxamide;
4-fluoro-3,6-dimethyl-N-(6-silaspiro[5.5]undecan-3-yl)-1H-pyrrolo[2,3-b]pyridine-2-carboxamide;
N-(1,1-dimethylsilepan-4-yl)-4,5-difluoro-6-methyl-1H-pyrrolo[2,3-b]pyridine-2-carboxamide;
4,5-difluoro-6-methyl-N-(5-silaspiro[4.5]decan-8-yl)-1H-pyrrolo[2,3-b]pyridine-2-carboxamide;
4,5-difluoro-6-methyl-N-(6-silaspiro[5.5]undecan-3-yl)-1H-pyrrolo[2,3-b]pyridine-2-carboxamide;
N-cyclooctyl-4,5-difluoro-6-methyl-1H-pyrrolo[2,3-b]pyridine-2-carboxamide;
5-chloro-N-(1,1-dimethylsilepan-4-yl)-4-fluoro-6-methyl-1H-pyrrolo[2,3-b]pyridine-2-carboxamide;
4,5-difluoro-N-[(1R,2R,3S,5R)-2-hydroxy-2,6,6-trimethyl-norpinan-3-yl]-6-methyl-1H-pyrrolo[2,3-b]pyridine-2-carboxamide;
N-(1,1-dimethylsilolan-3-yl)-4,5-difluoro-6-methyl-1H-pyrrolo[2,3-b]pyridine-2-carboxamide;
N-[(3R)-1,1-dimethylsilolan-3-yl]-4,5-difluoro-6-methyl-1H-pyrrolo[2,3-b]pyridine-2-carboxamide;
N-[(3S)-1,1-dimethylsilolan-3-yl]-4,5-difluoro-6-methyl-1H-pyrrolo[2,3-b]pyridine-2-carboxamide;
N-(1,1-dimethylsilocan-5-yl)-4,5-difluoro-6-methyl-1H-pyrrolo[2,3-b]pyridine-2-carboxamide;
4-chloro-N-(1,1-dimethylsilocan-5-yl)-6-methyl-1H-pyrrolo[2,3-b]pyridine-2-carboxamide;
4-chloro-N-(1,1-dimethylsilocan-5-ylidene)-6-methyl-1H-pyrrolo[2,3-b]pyridine-2-carboxamide;
5-chloro-N-(1,1-dimethylsilepan-4-yl)-4-fluoro-6-methyl-1H-pyrrolo[2,3-b]pyridine-2-carboxamide;
5-chloro-N-[(4R)-1,1-dimethylsilepan-4-yl]-4-fluoro-6-methyl-1H-pyrrolo[2,3-b]pyridine-2-carboxamide;
5-chloro-N-[(4S)-1,1-dimethylsilepan-4-yl]-4-fluoro-6-methyl-1H-pyrrolo[2,3-b]pyridine-2-carboxamide;
N-(1,1-dimethylsilinan-4-yl)-6-methoxy-1H-pyrrolo[2,3-b]pyridine-2-carboxamide;
N-(1,1-dimethylsilinan-4-yl)-6-phenyl-1H-pyrrolo[2,3-b]pyridine-2-carboxamide;
N-(1,1-dimethylsilinan-4-yl)-6-(3-pyridyl)-1H-pyrrolo[2,3-b]pyridine-2-carboxamide;
N-(1, 1-dimethylsilinan-4-yl)-5-methoxy-1H-pyrrolo[2,3-b]pyridine-2-carboxamide;
N-(1,1-dimethylsilinan-4-yl)-5-phenyl-1H-pyrrolo[2,3-b]pyridine-2-carboxamide;
N-(1,1-dimethylsilinan-4-yl)-5-(3-pyridyl)-1H-pyrrolo[2,3-b]pyridine-2-carboxamide;
4-chloro-N-(1,1-dimethylsilolan-3-yl)-6-methyl-1H-pyrrolo[2,3-b]pyridine-2-carboxamide;
4-chloro-N-[(3R)-1,1-dimethylsilolan-3-yl]-6-methyl-1H-pyrrolo[2,3-b]pyridine-2-carboxamide;
4-chloro-N-[(3S)-1,1-dimethylsilolan-3-yl]-6-methyl-1H-pyrrolo[2,3-b]pyridine-2-carboxamide;
5-(2-fluorophenyl)-N-(6-silaspiro[5.5]undecan-3-yl)-1H-pyrrolo[2,3-b]pyridine-2-carboxamide;
5-(3-pyridyl)-N-(6-silaspiro[5.5]undecan-3-yl)-1H-pyrrolo[2,3-b]pyridine-2-carboxamide;
N-(4,4-dimethylcyclohexyl)-6-methoxy-1H-pyrrolo[2,3-b]pyridine-2-carboxamide;
6-methoxy-N-(5-silaspiro[4.5]decan-8-yl)-1H-pyrrolo[2,3-b]pyridine-2-carboxamide;
6-methoxy-N-(6-silaspiro[5.5]undecan-3-yl)-1H-pyrrolo[2,3-b]pyridine-2-carboxamide;
4-chloro-N-(1,1-dimethylsilinan-4-yl)-6-methoxy-1H-pyrrolo[2,3-b]pyridine-2-carboxamide;
N-(1,1-dimethylsilepan-4-yl)-6-methoxy-1H-pyrrolo[2,3-b]pyridine-2-carboxamide;
(S)—N-(1,1-dimethylsilepan-4-yl)-6-methoxy-1H-pyrrolo[2,3-b]pyridine-2-carboxamide;
(R)—N-(1,1-dimethylsilepan-4-yl)-6-methoxy-1H-pyrrolo[2,3-b]pyridine-2-carboxamide;
6-(cyclobutoxy)-N-(1,1-dimethylsilinan-4-yl)-1H-pyrrolo[2,3-b]pyridine-2-carboxamide;
N-(1,1-dimethylsilinan-4-yl)-4-fluoro-6-methoxy-1H-pyrrolo[2,3-b]pyridine-2-carboxamide;
N-(1,1-dimethylsilocan-5-yl)-6-methoxy-1H-pyrrolo[2,3-b]pyridine-2-carboxamide;
N-(1,1-dimethylsilinan-4-yl)-4-fluoro-5,6-dimethyl-1H-pyrrolo[2,3-b]pyridine-2-carboxamide;
N-(1,1-dimethylsilepan-4-yl)-4-fluoro-5-methyl-1H-pyrrolo[2,3-c]pyridine-2-carboxamide;
4-chloro-N-(1,1-dimethylsilinan-4-yl)-5-methyl-1H-pyrrolo[2,3-c]pyridine-2-carboxamide;
4-fluoro-5-methyl-N-(5-silaspiro[4.5]decan-8-yl)-1H-pyrrolo[2,3-c]pyridine-2-carboxamide;
4-fluoro-5-methyl-N-(6-silaspiro[5.5]undecan-3-yl)-1H-pyrrolo[2,3-c]pyridine-2-carboxamide;
N-(1,1-dimethylsilinan-4-yl)-5-(trifluoromethyl)-1H-pyrrolo[2,3-c]pyridine-2-carboxamide;
N-(1,1-dimethylsilinan-4-yl)-4-fluoro-5-(trifluoromethyl)-1H-pyrrolo[2,3-c]pyridine-2-carboxamide;
4-chloro-N-(1,1-dimethylsilinan-4-yl)-5-(trifluoromethyl)-1H-pyrrolo[2,3-c]pyridine-2-carboxamide;
5-chloro-N-(1,1-dimethylsilinan-4-yl)-4-fluoro-1H-pyrrolo[2,3-c]pyridine-2-carboxamide;
5-chloro-N-(1,1-dimethylsilepan-4-yl)-4-fluoro-1H-pyrrolo[2,3-c]pyridine-2-carboxamide;
5-chloro-4-fluoro-N-(6-silaspiro[5.5]undecan-3-yl)-1H-pyrrolo[2,3-c]pyridine-2-carboxamide;
4,5-dichloro-N-(1,1-dimethylsilinan-4-yl)-1H-pyrrolo[2,3-c]pyridine-2-carboxamide;
4,5-dichloro-N-(1,1-dimethylsilepan-4-yl)-1H-pyrrolo[2,3-c]pyridine-2-carboxamide;
N-(1,1-dimethylsilinan-4-yl)-4-fluoro-5-methyl-1H-pyrrolo[2,3-c]pyridine-2-carboxamide;
N-(1,1-dimethylsilepan-4-yl)-4-fluoro-5-(trifluoromethyl)-1H-pyrrolo[2,3-c]pyridine-2-carboxamide;
4-fluoro-N-(5-silaspiro[4.5]decan-8-yl)-5-(trifluoromethyl)-1H-pyrrolo[2,3-c]pyridine-2-carboxamide;
4-fluoro-N-(6-silaspiro[5.5]undecan-3-yl)-5-(trifluoromethyl)-1H-pyrrolo[2,3-c]pyridine-2-carboxamide;
N-(1,1-dimethylsilinan-4-yl)-4-methyl-5-(trifluoromethyl)-1H-pyrrolo[2,3-c]pyridine-2-carboxamide;
N-(1,1-dimethylsilinan-4-yl)-4-methoxy-5-(trifluoromethyl)-1H-pyrrolo[2,3-c]pyridine-2-carboxamide;
N-(1,1-dimethylsilinan-4-yl)-4-Isopropoxy-1H-pyrrolo[2,3-c]pyridine-2-carboxamide;
N-(1,1-dimethylsilolan-3-yl)-5-(trifluoromethyl)-11H-pyrrolo[2,3-c]pyridine-2-carboxamide;
N-(1,1-dimethylsilinan-4-yl)-5-phenyl-1H-pyrrolo[2,3-c]pyridine-2-carboxamide;
N-(1, 1-dimethylsilepan-4-yl)-4-methyl-5-(trifluoromethyl)-1H-pyrrolo[2,3-c]pyridine-2-carboxamide;
N-(1,1-dimethylsilinan-4-yl)-5-methoxy-4-(trifluoromethyl)-1H-pyrrolo[2,3-c]pyridine-2-carboxamide;
N-(1,1-dimethylsilinan-4-yl)-4-fluoro-3,5-dimethyl-1H-pyrrolo[2,3-c]pyridine-2-carboxamide;
N-(1,1-dimethylsilepan-4-yl)-4-fluoro-3,5-dimethyl-1H-pyrrolo[2,3-c]pyridine-2-carboxamide;
or 6-(cyclopropoxy)-N-(1,1-dimethylsilinan-4-yl)-1H-pyrrolo[2,3-b]pyridine-2-carboxamide.

21. A pharmaceutical composition, comprising a compound of claim 1, or a pharmaceutically acceptable salt thereof, and one or more pharmaceutically acceptable carriers and/or additives.

22. The pharmaceutical composition according to claim 21, further comprising one or more additional anti-infective agents.

23. The pharmaceutical composition according to claim 21, wherein said additional anti-infective agent is rifampicin, rifabutin, rifapentene, isoniazid, ethambutol, kanamycin, amikacin, capreomycin, clofazimine, cycloserine, para-aminosalicylic acid, linezolid, sutezolid, bedaquiline, delamanid, pretomanid, moxifloxacin or levofloxacin, or combinations thereof.

24. A method of treating a mycobacterial infection, comprising the step of administering a therapeutically effective amount of a compound of claim 1, or a pharmaceutically acceptable salt thereof, to a patient in need thereof.

25. The method of claim 24, wherein the mycobacterial infection is caused by Mycobacterium tuberculosis, Mycobacterium avium, Mycobacterium kansasii, Mycobacterium abscessus or Mycobacterium chelonae.

26. The method of claim 24, wherein the mycobacterial infection is caused by Mycobacterium tuberculosis.
